SEQUENCE LISTING (236 sequences)

SEQ ID NO: 1 - protein, 383 aa, Artificial Sequence
  Synthetic Polypeptide
METPAQLLFLLLLWLPDTTGAEIYNKDGNKLDLFGKVDGLHYFSDDKGSDGDQTYMRIGFKGETQVNDQLTGYGQWEYQIQGNQTEGSNDSWTRVAFAGLKFADAGSFDYGRNYGVTYDVTSWTDVLPEFGGDTYGADNFMQQRGNGYATYRNTDFFGLVDGLDFALQYQGKNGSVSGENTNGRSLLNQNGDGYGGSLTYAIGEGFSVGGAITTSKRTADQNNTANARLYGNGDRATVYTGGLKYDANNIYLAAQYSQTYNATRFGTSNGSNPSTSYGFANKAQNFEVVAQYQFDFGLRPSVAYLQSKGKDISNGYGASYGDQDIVKYVDVGATYYFNKNMSTYVDYKINLLDKNDFTRDAGINTDDIVALGLVYQFHHHHHH

SEQ ID NO: 2 - RNA, 1149 nt, Artificial Sequence
  Synthetic Polynucleotide
auggaaaccccugcucagcugcuguuccugcugcugcuguggcugccugauacaacaggcgccgagaucuacaacaaggacggcaacaagcuggaccuguucggcaagguggacggccugcacuacuucagcgacgauaagggcuccgacggcgaccagaccuacaugagaaucggcuucaagggcgagacacaagugaacgaccagcugacaggcuacggccagugggaguaucagauccagggcaaucagaccgagggcagcaacgacagcuggaccagaguggcuuuugccggccugaaguuugccgaugccggcagcuuugacuacggcagaaauuacggcgugaccuacgacgugaccuccuggacagaugugcugccugaguuuggcggcgauaccuacggcgccgacaacuucaugcagcagagaggcaacggcuacgccaccuaccggaacaccgauuucuucggccugguggauggccuggauuucgcccugcaguaccagggcaagaauggcucuguguccggcgagaacaccaacggcagaagccugcugaaccagaacggcgacggauauggcggcagccugacauaugccaucggcgagggcuuuucugucggcggagccaucaccaccagcaagagaacagccgaccagaacaacaccgccaacgccagacuguacggcaacggcgauagagccacaguguauaccggcggacugaaguacgacgccaacaacaucuaccuggccgcucaguacagccagacauacaacgccaccagauucggcaccagcaacggcagcaaucccagcacaagcuacggcuucgccaacaaggcccagaacuuugaggugguggcccaguaccaguucgacuucggacugaggccuagcguggccuaccugcagagcaagggcaaagacaucagcaacggauacggcgccagcuacggcgaucaggacaucgugaaauacguggacgugggcgccaccuauuacuucaacaagaacaugagcaccuacguggacuacaagaucaaccugcuggacaagaacgacuucacccgcgacgccggcaucaacaccgaugauauuguggcccugggccucguguaccaguuucaccaccaccaucaccau

SEQ ID NO: 3 - RNA, 47 nt, Artificial Sequence
  Synthetic Polynucleotide
gggaaauaagagagaaaagaagaguaagaagaaauauaagagccacc

SEQ ID NO: 4 - RNA, 119 nt, Artificial Sequence
  Synthetic Polynucleotide
ugauaauaggcuggagccucgguggccaugcuucuugccccuugggccuccccccagccccuccuccccuuccugcacccguacccccguggucuuugaauaaagucugagugggcggc

SEQ ID NO: 5 - protein, 377 aa, Artificial Sequence
  Synthetic Polypeptide
METPAQLLFLLLLWLPDTTGAEIYNKDGNKLDLFGKVDGLHYFSDDKGSDGDQTYMRIGFKGETQVNDQLTGYGQWEYQIQGNQTEGSNDSWTRVAFAGLKFADAGSFDYGRNYGVTYDVTSWTDVLPEFGGDTYGADNFMQQRGNGYATYRNTDFFGLVDGLDFALQYQGKNGSVSGENTNGRSLLNQNGDGYGGSLTYAIGEGFSVGGAITTSKRTADQNNTANARLYGNGDRATVYTGGLKYDANNIYLAAQYSQTYNATRFGTSNGSNPSTSYGFANKAQNFEVVAQYQFDFGLRPSVAYLQSKGKDISNGYGASYGDQDIVKYVDVGATYYFNKNMSTYVDYKINLLDKNDFTRDAGINTDDIVALGLVYQF

SEQ ID NO: 6 - RNA, 1131 nt, Artificial Sequence
  Synthetic Polynucleotide
auggaaaccccugcucagcugcuguuccugcugcugcuguggcugccugauacaacaggcgccgagaucuacaacaaggacggcaacaagcuggaccuguucggcaagguggacggccugcacuacuucagcgacgauaagggcuccgacggcgaccagaccuacaugagaaucggcuucaagggcgagacacaagugaacgaccagcugacaggcuacggccagugggaguaucagauccagggcaaucagaccgagggcagcaacgacagcuggaccagaguggcuuuugccggccugaaguuugccgaugccggcagcuuugacuacggcagaaauuacggcgugaccuacgacgugaccuccuggacagaugugcugccugaguuuggcggcgauaccuacggcgccgacaacuucaugcagcagagaggcaacggcuacgccaccuaccggaacaccgauuucuucggccugguggauggccuggauuucgcccugcaguaccagggcaagaauggcucuguguccggcgagaacaccaacggcagaagccugcugaaccagaacggcgacggauauggcggcagccugacauaugccaucggcgagggcuuuucugucggcggagccaucaccaccagcaagagaacagccgaccagaacaacaccgccaacgccagacuguacggcaacggcgauagagccacaguguauaccggcggacugaaguacgacgccaacaacaucuaccuggccgcucaguacagccagacauacaacgccaccagauucggcaccagcaacggcagcaaucccagcacaagcuacggcuucgccaacaaggcccagaacuuugaggugguggcccaguaccaguucgacuucggacugaggccuagcguggccuaccugcagagcaagggcaaagacaucagcaacggauacggcgccagcuacggcgaucaggacaucgugaaauacguggacgugggcgccaccuauuacuucaacaagaacaugagcaccuacguggacuacaagaucaaccugcuggacaagaacgacuucacccgcgacgccggcaucaacaccgaugauauuguggcccugggccucguguaccaguuu

SEQ ID NO: 7 - protein, 367 aa, Artificial Sequence
  Synthetic Polypeptide
METPAQLLFLLLLWLPDTTGAEIYNKDGNKLDLYGKAVGRHVWTTTGDSKNADQTYAQIGFKGETQINTDLTGFGQWEYRTKADRAEGEQQNSNLVRLAFAGLKYAEVGSIDYGRNYGIVYDVESYTDMAPYFSGETWGGAYTDNYMTSRAGGLLTYRNSDFFGLVDGLSFGIQYQGKNQDNHSINSQNGDGVGYTMAYEFDGFGVTAAYSNSKRTNDQQDRDGNGDRAESWAVGAKYDANNVYLAAVYAETRNMSIVENTVTDTVEMANKTQNLEVVAQYQFDFGLRPAISYVQSKGKQLNGADGSADLAKYIQAGATYYFNKNMNVWVDYRFNLLDENDYSSSYVGTDDQAAVGITYQFHHHHHH

SEQ ID NO: 8 - RNA, 1101 nt, Artificial Sequence
  Synthetic Polynucleotide
auggaaaccccugcucagcugcuguuccugcugcugcuguggcugccugauacaacaggcgccgagaucuacaacaaggacggcaacaagcuggaccuguacggcaaagccgugggcagacacguguggacaaccaccggcgauagcaagaacgccgaccagacauaugcccagaucggcuucaagggcgagacacagaucaacaccgaccugaccggcuuuggccagugggaguacagaacaaaggccgacagagccgagggcgagcagcagaauucuaaucuugugcggcuggccuucgccggccugaaguaugcugaagugggcagcaucgacuacggccggaauuacggcaucguguacgacguggaaagcuacaccgacauggccccuuacuucagcggcgaaacauggggcggagccuacaccgauaacuacaugaccagcagagccggcggacugcugaccuacagaaacagcgauuucuucggccugguggacggccugagcuucggcauucaguaccagggcaagaaccaggacaaucacagcaucaacagccagaacggcgacggcgugggcuacacaauggccuacgaguucgauggcuuuggcgugacagccgccuacagcaacuccaagaggaccaacgaccagcaggacagagauggcaacggcgauagagccgaaucuugggccgugggcgccaaauacgacgccaacaauguguaucuggccgccguguacgccgagacacggaacaugagcaucguggagaacaccgugaccgacaccguggaaauggccaacaagacccagaaccuggaagugguggcccaguaccaguucgacuuuggacugaggcccgccaucagcuacgugcagucuaagggcaagcagcugaauggcgccgauggcucugcagaccuggccaaguauauucaggcuggcgccaccuacuacuucaacaagaacaugaacgugugggucgacuaccgguucaaccugcuggacgagaacgacuacagcagcuccuacgugggcaccgaugaucaggccgcugugggcaucaccuaccaguuccaccaccaccaucaccac

SEQ ID NO: 9 - protein, 361 aa, Artificial Sequence
  Synthetic Polypeptide
METPAQLLFLLLLWLPDTTGAEIYNKDGNKLDLYGKAVGRHVWTTTGDSKNADQTYAQIGFKGETQINTDLTGFGQWEYRTKADRAEGEQQNSNLVRLAFAGLKYAEVGSIDYGRNYGIVYDVESYTDMAPYFSGETWGGAYTDNYMTSRAGGLLTYRNSDFFGLVDGLSFGIQYQGKNQDNHSINSQNGDGVGYTMAYEFDGFGVTAAYSNSKRTNDQQDRDGNGDRAESWAVGAKYDANNVYLAAVYAETRNMSIVENTVTDTVEMANKTQNLEVVAQYQFDFGLRPAISYVQSKGKQLNGADGSADLAKYIQAGATYYFNKNMNVWVDYRFNLLDENDYSSSYVGTDDQAAVGITYQF

SEQ ID NO: 10 - RNA, 1083 nt, Artificial Sequence
  Synthetic Polynucleotide
auggaaaccccugcucagcugcuguuccugcugcugcuguggcugccugauacaacaggcgccgagaucuacaacaaggacggcaacaagcuggaccuguacggcaaagccgugggcagacacguguggacaaccaccggcgauagcaagaacgccgaccagacauaugcccagaucggcuucaagggcgagacacagaucaacaccgaccugaccggcuuuggccagugggaguacagaacaaaggccgacagagccgagggcgagcagcagaauucuaaucuugugcggcuggccuucgccggccugaaguaugcugaagugggcagcaucgacuacggccggaauuacggcaucguguacgacguggaaagcuacaccgacauggccccuuacuucagcggcgaaacauggggcggagccuacaccgauaacuacaugaccagcagagccggcggacugcugaccuacagaaacagcgauuucuucggccugguggacggccugagcuucggcauucaguaccagggcaagaaccaggacaaucacagcaucaacagccagaacggcgacggcgugggcuacacaauggccuacgaguucgauggcuuuggcgugacagccgccuacagcaacuccaagaggaccaacgaccagcaggacagagauggcaacggcgauagagccgaaucuugggccgugggcgccaaauacgacgccaacaauguguaucuggccgccguguacgccgagacacggaacaugagcaucguggagaacaccgugaccgacaccguggaaauggccaacaagacccagaaccuggaagugguggcccaguaccaguucgacuuuggacugaggcccgccaucagcuacgugcagucuaagggcaagcagcugaauggcgccgauggcucugcagaccuggccaaguauauucaggcuggcgccaccuacuacuucaacaagaacaugaacgugugggucgacuaccgguucaaccugcuggacgagaacgacuacagcagcuccuacgugggcaccgaugaucaggccgcugugggcaucaccuaccaguuc

SEQ ID NO: 11 - protein, 367 aa, Artificial Sequence
  Synthetic Polypeptide
METPAQLLFLLLLWLPDTTGAEIYNKDGNKLDLYGKAVGRHVWTTTGDSKNADQTYAQIGFKGETQINTDLTGFGQWEYRTKADRAEGEQQNSNLVRLAFAGLKYAEVGSIDYGRNYGIVYDVESYTDMAPYFSGETWGGAYTDNYMTSRAGGLLTYRNSDFFGLVDGLSFGIQYQGKNQDNHSINSQNGDGVGYTMAYEFDGFGVTAAYSNSKRTNDQQDRDGNGDRAESWAVGAKYDANNVYLAAVYAETRNMAIVENTVTDTVEMANKAQNLEVVAQYQFDFGLRPAISYVQSKGKQLNGADGSADLAKYIQAGATYYFNKNMNVWVDYRFNLLDENDYSSSYVGTDDQAAVGITYQFHHHHHH

SEQ ID NO: 12 - RNA, 1101 nt, Artificial Sequence
  Synthetic Polynucleotide
auggaaaccccugcucagcugcuguuccugcugcugcuguggcugccugauacaacaggcgccgagaucuacaacaaggacggcaacaagcuggaccuguacggcaaagccgugggcagacacguguggacaaccaccggcgauagcaagaacgccgaccagacauaugcccagaucggcuucaagggcgagacacagaucaacaccgaccugaccggcuuuggccagugggaguacagaacaaaggccgacagagccgagggcgagcagcagaauucuaaucuugugcggcuggccuucgccggccugaaguaugcugaagugggcagcaucgacuacggccggaauuacggcaucguguacgacguggaaagcuacaccgacauggccccuuacuucagcggcgaaacauggggcggagccuacaccgauaacuacaugaccagcagagccggcggacugcugaccuacagaaacagcgauuucuucggccugguggacggccugagcuucggcauucaguaccagggcaagaaccaggacaaucacagcaucaacagccagaacggcgacggcgugggcuacacaauggccuacgaguucgauggcuuuggcgugacagccgccuacagcaacuccaagaggaccaacgaccagcaggacagagauggcaacggcgauagagccgaaucuugggccgugggcgccaaauacgacgccaacaauguguaucuggccgccguguacgccgagacaagaaacauggccaucguggagaacaccgugaccgacaccguggaaauggccaacaaggcccagaaccuggaagugguggcccaguaccaguucgacuuuggacugaggcccgccaucagcuacgugcagucuaagggcaagcagcugaauggcgccgauggcucugcagaccuggccaaguauauucaggcuggcgccaccuacuacuucaacaagaacaugaacgugugggucgacuaccgguucaaccugcuggacgagaacgacuacagcagcuccuacgugggcaccgaugaucaggccgcugugggcaucaccuaccaguuccaccaccaccaucaccac

SEQ ID NO: 13 - protein, 361 aa, Artificial Sequence
  Synthetic Polypeptide
METPAQLLFLLLLWLPDTTGAEIYNKDGNKLDLYGKAVGRHVWTTTGDSKNADQTYAQIGFKGETQINTDLTGFGQWEYRTKADRAEGEQQNSNLVRLAFAGLKYAEVGSIDYGRNYGIVYDVESYTDMAPYFSGETWGGAYTDNYMTSRAGGLLTYRNSDFFGLVDGLSFGIQYQGKNQDNHSINSQNGDGVGYTMAYEFDGFGVTAAYSNSKRTNDQQDRDGNGDRAESWAVGAKYDANNVYLAAVYAETRNMAIVENTVTDTVEMANKAQNLEVVAQYQFDFGLRPAISYVQSKGKQLNGADGSADLAKYIQAGATYYFNKNMNVWVDYRFNLLDENDYSSSYVGTDDQAAVGITYQF

SEQ ID NO: 14 - RNA, 1083 nt, Artificial Sequence
  Synthetic Polynucleotide
auggaaaccccugcucagcugcuguuccugcugcugcuguggcugccugauacaacaggcgccgagaucuacaacaaggacggcaacaagcuggaccuguacggcaaagccgugggcagacacguguggacaaccaccggcgauagcaagaacgccgaccagacauaugcccagaucggcuucaagggcgagacacagaucaacaccgaccugaccggcuuuggccagugggaguacagaacaaaggccgacagagccgagggcgagcagcagaauucuaaucuugugcggcuggccuucgccggccugaaguaugcugaagugggcagcaucgacuacggccggaauuacggcaucguguacgacguggaaagcuacaccgacauggccccuuacuucagcggcgaaacauggggcggagccuacaccgauaacuacaugaccagcagagccggcggacugcugaccuacagaaacagcgauuucuucggccugguggacggccugagcuucggcauucaguaccagggcaagaaccaggacaaucacagcaucaacagccagaacggcgacggcgugggcuacacaauggccuacgaguucgauggcuuuggcgugacagccgccuacagcaacuccaagaggaccaacgaccagcaggacagagauggcaacggcgauagagccgaaucuugggccgugggcgccaaauacgacgccaacaauguguaucuggccgccguguacgccgagacaagaaacauggccaucguggagaacaccgugaccgacaccguggaaauggccaacaaggcccagaaccuggaagugguggcccaguaccaguucgacuuuggacugaggcccgccaucagcuacgugcagucuaagggcaagcagcugaauggcgccgauggcucugcagaccuggccaaguauauucaggcuggcgccaccuacuacuucaacaagaacaugaacgugugggucgacuaccgguucaaccugcuggacgagaacgacuacagcagcuccuacgugggcaccgaugaucaggccgcugugggcaucaccuaccaguuc

SEQ ID NO: 15 - protein, 367 aa, Artificial Sequence
  Synthetic Polypeptide
METPAQLLFLLLLWLPDTTGAEVYNKDGNKLDLYGKVHAQHYFSDDNGSDGDKTYARLGFKGETQINDQLTGFGQWEYEFKGNRTESQGADKDKTRLAFAGLKFADYGSFDYGRNYGVAYDIGAWTDVLPEFGGDTWTQTDVFMTGRTTGVATYRNTDFFGLVEGLNFAAQYQGKNDRDGAYESNGDGFGLSATYEYEGFGVGAAYAKSDRTNNQVKAASNLNAAGKNAEVWAAGLKYDANNIYLATTYSETLNMTTFGEDAAGDAFIANKTQNFEAVAQYQFDFGLRPSIAYLKSKGKNLGTYGDQDLVEYIDVGATYYFNKNMSTFVDYKINLLDDSDFTKAAKVSTDNIVAVGLNYQFHHHHHH

SEQ ID NO: 16 - RNA, 1101 nt, Artificial Sequence
  Synthetic Polynucleotide
auggaaaccccugcucagcugcuguuccugcugcugcuguggcugccugauacaacaggcgccgagguguacaacaaggacggcaacaagcuggaccuguacggcaaagugcacgcccagcacuacuucagcgacgacaauggcagcgacggcgacaagacauaugcccggcuuggcuucaagggcgagacacagaucaacgaccagcugaccggcuuuggccagugggaguacgaguucaagggcaacagaaccgagagccagggcgccgacaaggacaagaccagacuggccuuugccggccugaaguucgccgauuacggcagcuuugacuacggccggaauuacggcguggccuacgauaucggagccuggacagaugugcugccugaguuuggcggcgacaccuggacacagaccgacguguucaugaccggcagaacaacuggcguggccaccuaccggaacaccgauuucuuuggccugguggaaggccugaacuuugccgcucaguaccagggcaagaacgacagagauggcgccuacgagucuaacggcgacggcuuuggacugagcgccaccuacgaguacgaaggcuuuggagugggcgcugccuacgccaagagcgacaggaccaacaaucaagugaaggccgccagcaaccugaacgccgcuggaaagaaugccgaagugugggccgcuggacugaaguacgacgccaacaacaucuaccuggccaccaccuacagcgagacacugaacaugaccaccuucggcgaagaugccgcuggcgacgccuuuaucgccaacaagacccagaacuucgaggcuguggcccaguaccaguucgacuucggacugaggcccucuaucgccuaccugaaguccaagggaaagaaccugggcaccuacggcgaccaggaccugguugaguauaucgaugugggagccaccuacuacuucaacaagaauaugagcaccuucguggacuacaagaucaaccugcuggacgacagcgacuucaccaaagccgccaagguguccaccgacaacauuguggccgugggccugaauuaccaguuccaccaccaccaucaccac

SEQ ID NO: 17 - protein, 361 aa, Artificial Sequence
  Synthetic Polypeptide
METPAQLLFLLLLWLPDTTGAEVYNKDGNKLDLYGKVHAQHYFSDDNGSDGDKTYARLGFKGETQINDQLTGFGQWEYEFKGNRTESQGADKDKTRLAFAGLKFADYGSFDYGRNYGVAYDIGAWTDVLPEFGGDTWTQTDVFMTGRTTGVATYRNTDFFGLVEGLNFAAQYQGKNDRDGAYESNGDGFGLSATYEYEGFGVGAAYAKSDRTNNQVKAASNLNAAGKNAEVWAAGLKYDANNIYLATTYSETLNMTTFGEDAAGDAFIANKTQNFEAVAQYQFDFGLRPSIAYLKSKGKNLGTYGDQDLVEYIDVGATYYFNKNMSTFVDYKINLLDDSDFTKAAKVSTDNIVAVGLNYQF

SEQ ID NO: 18 - RNA, 1083 nt, Artificial Sequence
  Synthetic Polynucleotide
auggaaaccccugcucagcugcuguuccugcugcugcuguggcugccugauacaacaggcgccgagguguacaacaaggacggcaacaagcuggaccuguacggcaaagugcacgcccagcacuacuucagcgacgacaauggcagcgacggcgacaagacauaugcccggcuuggcuucaagggcgagacacagaucaacgaccagcugaccggcuuuggccagugggaguacgaguucaagggcaacagaaccgagagccagggcgccgacaaggacaagaccagacuggccuuugccggccugaaguucgccgauuacggcagcuuugacuacggccggaauuacggcguggccuacgauaucggagccuggacagaugugcugccugaguuuggcggcgacaccuggacacagaccgacguguucaugaccggcagaacaacuggcguggccaccuaccggaacaccgauuucuuuggccugguggaaggccugaacuuugccgcucaguaccagggcaagaacgacagagauggcgccuacgagucuaacggcgacggcuuuggacugagcgccaccuacgaguacgaaggcuuuggagugggcgcugccuacgccaagagcgacaggaccaacaaucaagugaaggccgccagcaaccugaacgccgcuggaaagaaugccgaagugugggccgcuggacugaaguacgacgccaacaacaucuaccuggccaccaccuacagcgagacacugaacaugaccaccuucggcgaagaugccgcuggcgacgccuuuaucgccaacaagacccagaacuucgaggcuguggcccaguaccaguucgacuucggacugaggcccucuaucgccuaccugaaguccaagggaaagaaccugggcaccuacggcgaccaggaccugguugaguauaucgaugugggagccaccuacuacuucaacaagaauaugagcaccuucguggacuacaagaucaaccugcuggacgacagcgacuucaccaaagccgccaagguguccaccgacaacauuguggccgugggccugaauuaccaguuc

SEQ ID NO: 19 - protein, 383 aa, Artificial Sequence
  Synthetic Polypeptide
METPAQLLFLLLLWLPDTTGAEIYNKDGNKLDLFGKVDGLHYFSDDKGSDGDQTYMRIGFKGETQVNDQLTGYGQWEYQIQGNQAEGSNDAWTRVAFAGLKFADAGSFDYGRNYGVTYDVTSWTDVLPEFGGDTYGADNFMQQRGNGYATYRNTDFFGLVDGLDFALQYQGKNGAVSGENTNGRSLLNQNGDGYGGSLTYAIGEGFSVGGAITTSKRTADQNNAANARLYGNGDRATVYTGGLKYDANNIYLAAQYSQTYNAARFGTSNGANPSTSYGFANKAQNFEVVAQYQFDFGLRPSVAYLQSKGKDISNGYGASYGDQDIVKYVDVGATYYFNKNMSTYVDYKINLLDKNDFTRDAGINTDDIVALGLVYQFHHHHHH

SEQ ID NO: 20 - RNA, 1149 nt, Artificial Sequence
  Synthetic Polynucleotide
auggaaaccccugcucagcugcuguuccugcugcugcuguggcugccugauacaacaggcgccgagaucuacaacaaggacggcaacaagcuggaccuguucggcaagguggacggccugcacuacuucagcgacgauaagggcuccgacggcgaccagaccuacaugagaaucggcuucaagggcgagacacaagugaacgaccagcugacaggcuacggccagugggaguaucagauccagggcaaucaggccgagggcagcaacgacgcauggaccagaguggcuuuugccggccugaaguuugccgaugccggcagcuuugacuacggcagaaauuacggcgugaccuacgacgugaccuccuggacagaugugcugccugaguuuggcggcgauaccuacggcgccgacaacuucaugcagcagagaggcaacggcuacgccaccuaccggaacaccgauuucuucggccugguggauggccuggauuucgcccugcaguaccagggcaagaauggcgcuguguccggcgagaacaccaacggcagaagccugcugaaccagaacggcgacggauauggcggcagccugacauaugccaucggcgagggcuuuucugucggcggagccaucaccaccagcaagagaacagccgaccagaacaacgccgccaacgccagacuguacggcaacggcgauagagccacaguguauaccggcggacugaaguacgacgccaacaacaucuaccuggccgcucaguacagccagacauacaacgccgccagauucggcaccagcaacggcgcaaaucccagcacaagcuacggcuucgccaacaaggcccagaacuuugaggugguggcccaguaccaguucgacuucggacugaggccuagcguggccuaccugcagagcaagggcaaagacaucagcaacggauacggcgccagcuacggcgaucaggacaucgugaaauacguggacgugggcgccaccuauuacuucaacaagaacaugagcaccuacguggacuacaagaucaaccugcuggacaagaacgacuucacccgcgacgccggcaucaacaccgaugauauuguggcccugggccucguguaccaguuucaccaccaccaucaccau

SEQ ID NO: 21 - protein, 377 aa, Artificial Sequence
  Synthetic Polypeptide
METPAQLLFLLLLWLPDTTGAEIYNKDGNKLDLFGKVDGLHYFSDDKGSDGDQTYMRIGFKGETQVNDQLTGYGQWEYQIQGNQAEGSNDAWTRVAFAGLKFADAGSFDYGRNYGVTYDVTSWTDVLPEFGGDTYGADNFMQQRGNGYATYRNTDFFGLVDGLDFALQYQGKNGAVSGENTNGRSLLNQNGDGYGGSLTYAIGEGFSVGGAITTSKRTADQNNAANARLYGNGDRATVYTGGLKYDANNIYLAAQYSQTYNAARFGTSNGANPSTSYGFANKAQNFEVVAQYQFDFGLRPSVAYLQSKGKDISNGYGASYGDQDIVKYVDVGATYYFNKNMSTYVDYKINLLDKNDFTRDAGINTDDIVALGLVYQF

SEQ ID NO: 22 - RNA, 1131 nt, Artificial Sequence
  Synthetic Polynucleotide
auggaaaccccugcucagcugcuguuccugcugcugcuguggcugccugauacaacaggcgccgagaucuacaacaaggacggcaacaagcuggaccuguucggcaagguggacggccugcacuacuucagcgacgauaagggcuccgacggcgaccagaccuacaugagaaucggcuucaagggcgagacacaagugaacgaccagcugacaggcuacggccagugggaguaucagauccagggcaaucaggccgagggcagcaacgacgcauggaccagaguggcuuuugccggccugaaguuugccgaugccggcagcuuugacuacggcagaaauuacggcgugaccuacgacgugaccuccuggacagaugugcugccugaguuuggcggcgauaccuacggcgccgacaacuucaugcagcagagaggcaacggcuacgccaccuaccggaacaccgauuucuucggccugguggauggccuggauuucgcccugcaguaccagggcaagaauggcgcuguguccggcgagaacaccaacggcagaagccugcugaaccagaacggcgacggauauggcggcagccugacauaugccaucggcgagggcuuuucugucggcggagccaucaccaccagcaagagaacagccgaccagaacaacgccgccaacgccagacuguacggcaacggcgauagagccacaguguauaccggcggacugaaguacgacgccaacaacaucuaccuggccgcucaguacagccagacauacaacgccgccagauucggcaccagcaacggcgcaaaucccagcacaagcuacggcuucgccaacaaggcccagaacuuugaggugguggcccaguaccaguucgacuucggacugaggccuagcguggccuaccugcagagcaagggcaaagacaucagcaacggauacggcgccagcuacggcgaucaggacaucgugaaauacguggacgugggcgccaccuauuacuucaacaagaacaugagcaccuacguggacuacaagaucaaccugcuggacaagaacgacuucacccgcgacgccggcaucaacaccgaugauauuguggcccugggccucguguaccaguuu

SEQ ID NO: 23 - protein, 267 aa, Artificial Sequence
  Synthetic Polypeptide
MIPMSETKNELEILLEKAATEPAHRSAFFRTLLESTVWVPGSAAEGEAIVEDSALDLQHWEKEDGTTVIPFFTSLEALQQAVEDEQAFVVMPARTLFEMTLGETLFLNAKLPTGKEFMPREISLLLAEEGSPLSTQEVLEGGESLILSEVAEPPSQMIDSLTTLFKTIKPVKRAFLCAIKEHADAQPNLLIGIEADGEIEEIIHAAGNVATDTLPGDEPIDICQVRKGAQGISHFITEHIAPFYERRWGGFLRDFKQNRIIHHHHHH

SEQ ID NO: 24 - RNA, 801 nt, Artificial Sequence
  Synthetic Polynucleotide
augauccccaugagcgagacaaagaacgagcuggaaauccugcucgagaaggccgccacagagccugcucacagaagcgcuuucuucagaacccugcuggaaagcaccgugugggugccaggaucugcugcugaaggcgaagccaucguggaagauagcgcccuggaucugcagcacugggagaaagaggacggaaccacagucaucccauucuucaccagccuggaagcccugcagcaggcuguggaagaugagcaggccuucguggucaugcccgccagaacacuguucgagaugacccugggcgagacacuguuccugaacgccaaacugcccaccggcaaagaauucaugcccagagagaucucccugcugcuggccgaggaaggaucuccucugagcacacaagaggugcuggaaggcggcgagagccugauucugucugaaguggccgagccuccuagccagaugaucgacagccugaccacacuguucaagaccaucaagcccgugaagcgggccuuccugugcgccaucaaagaacacgcugacgcccagccuaaccugcugaucggaauugaggccgacggcgagaucgaggaaaucauccacgccgcuggaaacguggccaccgauacacugccuggcgacgagccuaucgacaucugccaaguucggaaaggcgcccagggaaucagccacuucaucaccgaacacauugccccauucuacgagcggagauggggcggcuuccugagagacuucaagcagaaccggaucauccaccaccaccaucaccac

SEQ ID NO: 25 - protein, 261 aa, Artificial Sequence
  Synthetic Polypeptide
MIPMSETKNELEILLEKAATEPAHRSAFFRTLLESTVWVPGSAAEGEAIVEDSALDLQHWEKEDGTTVIPFFTSLEALQQAVEDEQAFVVMPARTLFEMTLGETLFLNAKLPTGKEFMPREISLLLAEEGSPLSTQEVLEGGESLILSEVAEPPSQMIDSLTTLFKTIKPVKRAFLCAIKEHADAQPNLLIGIEADGEIEEIIHAAGNVATDTLPGDEPIDICQVRKGAQGISHFITEHIAPFYERRWGGFLRDFKQNRII

SEQ ID NO: 26 - RNA, 783 nt, Artificial Sequence
  Synthetic Polynucleotide
augauccccaugagcgagacaaagaacgagcuggaaauccugcucgagaaggccgccacagagccugcucacagaagcgcuuucuucagaacccugcuggaaagcaccgugugggugccaggaucugcugcugaaggcgaagccaucguggaagauagcgcccuggaucugcagcacugggagaaagaggacggaaccacagucaucccauucuucaccagccuggaagcccugcagcaggcuguggaagaugagcaggccuucguggucaugcccgccagaacacuguucgagaugacccugggcgagacacuguuccugaacgccaaacugcccaccggcaaagaauucaugcccagagagaucucccugcugcuggccgaggaaggaucuccucugagcacacaagaggugcuggaaggcggcgagagccugauucugucugaaguggccgagccuccuagccagaugaucgacagccugaccacacuguucaagaccaucaagcccgugaagcgggccuuccugugcgccaucaaagaacacgcugacgcccagccuaaccugcugaucggaauugaggccgacggcgagaucgaggaaaucauccacgccgcuggaaacguggccaccgauacacugccuggcgacgagccuaucgacaucugccaaguucggaaaggcgcccagggaaucagccacuucaucaccgaacacauugccccauucuacgagcggagauggggcggcuuccugagagacuucaagcagaaccggaucauc

SEQ ID NO: 27 - protein, 286 aa, Artificial Sequence
  Synthetic Polypeptide
METPAQLLFLLLLWLPDTTGIPMSETKNELEILLEKAATEPAHRSAFFRTLLESTVWVPGSAAEGEAIVEDSALDLQHWEKEDGTTVIPFFTSLEALQQAVEDEQAFVVMPARTLFEMTLGETLFLNAKLPTGKEFMPREISLLLAEEGSPLSTQEVLEGGESLILSEVAEPPSQMIDSLTTLFKTIKPVKRAFLCAIKEHADAQPNLLIGIEADGEIEEIIHAAGNVATDTLPGDEPIDICQVRKGAQGISHFITEHIAPFYERRWGGFLRDFKQNRIIHHHHHH

SEQ ID NO: 28 - RNA, 858 nt, Artificial Sequence
  Synthetic Polynucleotide
auggaaaccccugcucagcugcuguuccugcugcugcuguggcugccugauacaacaggcauccccaugagcgagacaaagaacgagcuggaaauccugcucgagaaggccgccacagagccugcucacagaagcgcuuucuucagaacccugcuggaaagcaccgugugggugccaggaucugcugcugaaggcgaagccaucguggaagauagcgcccuggaucugcagcacugggagaaagaggacggaaccacagucaucccauucuucaccagccuggaagcccugcagcaggcuguggaagaugagcaggccuucguggucaugcccgccagaacacuguucgagaugacccugggcgagacacuguuccugaacgccaaacugcccaccggcaaagaauucaugcccagagagaucucccugcugcuggccgaggaaggaucuccucugagcacacaagaggugcuggaaggcggcgagagccugauucugucugaaguggccgagccuccuagccagaugaucgacagccugaccacacuguucaagaccaucaagcccgugaagcgggccuuccugugcgccaucaaagaacacgcugacgcccagccuaaccugcugaucggaauugaggccgacggcgagaucgaggaaaucauccacgccgcuggaaacguggccaccgauacacugccuggcgacgagccuaucgacaucugccaaguucggaaaggcgcccagggaaucagccacuucaucaccgaacacauugccccauucuacgagcggagauggggcggcuuccugagagacuucaagcagaaccggaucauccaccaccaccaucaccac

SEQ ID NO: 29 - protein, 280 aa, Artificial Sequence
  Synthetic Polypeptide
METPAQLLFLLLLWLPDTTGIPMSETKNELEILLEKAATEPAHRSAFFRTLLESTVWVPGSAAEGEAIVEDSALDLQHWEKEDGTTVIPFFTSLEALQQAVEDEQAFVVMPARTLFEMTLGETLFLNAKLPTGKEFMPREISLLLAEEGSPLSTQEVLEGGESLILSEVAEPPSQMIDSLTTLFKTIKPVKRAFLCAIKEHADAQPNLLIGIEADGEIEEIIHAAGNVATDTLPGDEPIDICQVRKGAQGISHFITEHIAPFYERRWGGFLRDFKQNRII

SEQ ID NO: 30 - RNA, 840 nt, Artificial Sequence
  Synthetic Polynucleotide
auggaaaccccugcucagcugcuguuccugcugcugcuguggcugccugauacaacaggcauccccaugagcgagacaaagaacgagcuggaaauccugcucgagaaggccgccacagagccugcucacagaagcgcuuucuucagaacccugcuggaaagcaccgugugggugccaggaucugcugcugaaggcgaagccaucguggaagauagcgcccuggaucugcagcacugggagaaagaggacggaaccacagucaucccauucuucaccagccuggaagcccugcagcaggcuguggaagaugagcaggccuucguggucaugcccgccagaacacuguucgagaugacccugggcgagacacuguuccugaacgccaaacugcccaccggcaaagaauucaugcccagagagaucucccugcugcuggccgaggaaggaucuccucugagcacacaagaggugcuggaaggcggcgagagccugauucugucugaaguggccgagccuccuagccagaugaucgacagccugaccacacuguucaagaccaucaagcccgugaagcgggccuuccugugcgccaucaaagaacacgcugacgcccagccuaaccugcugaucggaauugaggccgacggcgagaucgaggaaaucauccacgccgcuggaaacguggccaccgauacacugccuggcgacgagccuaucgacaucugccaaguucggaaaggcgcccagggaaucagccacuucaucaccgaacacauugccccauucuacgagcggagauggggcggcuuccugagagacuucaagcagaaccggaucauc

SEQ ID NO: 31 - protein, 512 aa, Artificial Sequence
  Synthetic Polypeptide
MAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDPITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKTGAITAKTTTYTDGTGVAQTGAVKFGGANGKSEVVTATDGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLRHHHHHH

SEQ ID NO: 32 - RNA, 1536 nt, Artificial Sequence
  Synthetic Polynucleotide
auggcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcaaucugccaauggcacaaacagccaguccgaccuggauuccauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaaagagaucuccagcaagacccugggccucgacaagcugaacgugcaggaugccuacacacccaaagaaaccgccgugaccguggacaagaccaccuacaagaacggcacagaccccaucacagcccagagcaacaccgauauccagaccgccauuggaggcggagcuacagguguuacaggcgccgacaucaaguucaaggacggccaguacuaccuggacgugaaaggcggagcaucugccggcguguacaaggccacauacgacgaaaccaccaagaaagugaacaucgacaccaccgacaagaccccucuggccacagcugaagccacagccauuagaggcaccgccacaaucacccacaaccagaucgccgaagugaccaaagaaggcguggacaccacaaccguggcugcucaacuugcugcugcuggcguuaccggcgcugacaaggauaauaccagccuggucaagcugagcuucgaggacaagaauggcaaagugaucgacggcggcuacgccgugaagaugggcgacgauuucuacgccgccaccuacgaugagaagaccggcgccauuaccgccaagaccacaaccuacacagauggcacaggcguggcacagacaggggccgugaaauuuggaggcgccaacggcaagagcgaggucgugacagcuaccgacggcaagacauaccuggccagcgaucuggacaagcacaacuucagaacaggcggcgagcugaaagaagugaacacagacaagaccgagaauccgcugcagaagaucgacgcugcucuggcacaaguggacacccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugagacaccaccaccaucaccau

SEQ ID NO: 33 - protein, 506 aa, Artificial Sequence
  Synthetic Polypeptide
MAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDPITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKTGAITAKTTTYTDGTGVAQTGAVKFGGANGKSEVVTATDGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 34 - RNA, 1518 nt, Artificial Sequence
  Synthetic Polynucleotide
auggcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcaaucugccaauggcacaaacagccaguccgaccuggauuccauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaaagagaucuccagcaagacccugggccucgacaagcugaacgugcaggaugccuacacacccaaagaaaccgccgugaccguggacaagaccaccuacaagaacggcacagaccccaucacagcccagagcaacaccgauauccagaccgccauuggaggcggagcuacagguguuacaggcgccgacaucaaguucaaggacggccaguacuaccuggacgugaaaggcggagcaucugccggcguguacaaggccacauacgacgaaaccaccaagaaagugaacaucgacaccaccgacaagaccccucuggccacagcugaagccacagccauuagaggcaccgccacaaucacccacaaccagaucgccgaagugaccaaagaaggcguggacaccacaaccguggcugcucaacuugcugcugcuggcguuaccggcgcugacaaggauaauaccagccuggucaagcugagcuucgaggacaagaauggcaaagugaucgacggcggcuacgccgugaagaugggcgacgauuucuacgccgccaccuacgaugagaagaccggcgccauuaccgccaagaccacaaccuacacagauggcacaggcguggcacagacaggggccgugaaauuuggaggcgccaacggcaagagcgaggucgugacagcuaccgacggcaagacauaccuggccagcgaucuggacaagcacaacuucagaacaggcggcgagcugaaagaagugaacacagacaagaccgagaauccgcugcagaagaucgacgcugcucuggcacaaguggacacccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugaga

SEQ ID NO: 35 - protein, 531 aa, Artificial Sequence
  Synthetic Polypeptide
METPAQLLFLLLLWLPDTTGAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDPITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKTGAITAKTTTYTDGTGVAQTGAVKFGGANGKSEVVTATDGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLRHHHHHH

SEQ ID NO: 36 - RNA, 1593 nt, Artificial Sequence
  Synthetic Polynucleotide
auggaaaccccugcucagcugcuguuccugcugcugcuguggcugccugauacaacaggcgcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcaaucugccaauggcacaaacagccaguccgaccuggauuccauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaaagagaucuccagcaagacccugggccucgacaagcugaacgugcaggaugccuacacacccaaagaaaccgccgugaccguggacaagaccaccuacaagaacggcacagaccccaucacagcccagagcaacaccgauauccagaccgccauuggaggcggagcuacagguguuacaggcgccgacaucaaguucaaggacggccaguacuaccuggacgugaaaggcggagcaucugccggcguguacaaggccacauacgacgaaaccaccaagaaagugaacaucgacaccaccgacaagaccccucuggccacagcugaagccacagccauuagaggcaccgccacaaucacccacaaccagaucgccgaagugaccaaagaaggcguggacaccacaaccguggcugcucaacuugcugcugcuggcguuaccggcgcugacaaggauaauaccagccuggucaagcugagcuucgaggacaagaauggcaaagugaucgacggcggcuacgccgugaagaugggcgacgauuucuacgccgccaccuacgaugagaagaccggcgccauuaccgccaagaccacaaccuacacagauggcacaggcguggcacagacaggggccgugaaauuuggaggcgccaacggcaagagcgaggucgugacagcuaccgacggcaagacauaccuggccagcgaucuggacaagcacaacuucagaacaggcggcgagcugaaagaagugaacacagacaagaccgagaauccgcugcagaagaucgacgcugcucuggcacaaguggacacccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugagacaccaccaccaucaccau

SEQ ID NO: 37 - protein, 525 aa, Artificial Sequence
  Synthetic Polypeptide
METPAQLLFLLLLWLPDTTGAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDPITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKTGAITAKTTTYTDGTGVAQTGAVKFGGANGKSEVVTATDGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 38 - RNA, 1575 nt, Artificial Sequence
  Synthetic Polynucleotide
auggaaaccccugcucagcugcuguuccugcugcugcuguggcugccugauacaacaggcgcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcaaucugccaauggcacaaacagccaguccgaccuggauuccauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaaagagaucuccagcaagacccugggccucgacaagcugaacgugcaggaugccuacacacccaaagaaaccgccgugaccguggacaagaccaccuacaagaacggcacagaccccaucacagcccagagcaacaccgauauccagaccgccauuggaggcggagcuacagguguuacaggcgccgacaucaaguucaaggacggccaguacuaccuggacgugaaaggcggagcaucugccggcguguacaaggccacauacgacgaaaccaccaagaaagugaacaucgacaccaccgacaagaccccucuggccacagcugaagccacagccauuagaggcaccgccacaaucacccacaaccagaucgccgaagugaccaaagaaggcguggacaccacaaccguggcugcucaacuugcugcugcuggcguuaccggcgcugacaaggauaauaccagccuggucaagcugagcuucgaggacaagaauggcaaagugaucgacggcggcuacgccgugaagaugggcgacgauuucuacgccgccaccuacgaugagaagaccggcgccauuaccgccaagaccacaaccuacacagauggcacaggcguggcacagacaggggccgugaaauuuggaggcgccaacggcaagagcgaggucgugacagcuaccgacggcaagacauaccuggccagcgaucuggacaagcacaacuucagaacaggcggcgagcugaaagaagugaacacagacaagaccgagaauccgcugcagaagaucgacgcugcucuggcacaaguggacacccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugaga

SEQ ID NO: 39 - protein, 501 aa, Artificial Sequence
  Synthetic Polypeptide
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANNGETIDIDLKQINSQTLGLDTLNVQKKYDVKSEAVTPSATLSTTALDGAGLKTGTGSTTDTGSIKDGKVYYNSTSKNYYVEVEFTDATDQTNKGGFYKVNVADDGAVTMTAATTKEATTPTGITEVTQVQKPVAAPAAIQAQLTAAHVTGADTAEMVKMSYTDKNGKTIDGGFGVKVGADIYAATKNKDGSFSINTTEYTDKDGNTKTALNQLGGADGKTEVVSIDGKTYNASKAAGHNFKAQPELAEAAAATTENPLAKIDAALAQVDALRSDLGAVQNRFNSAITNLGNTVNNLSSARSRIEDSDYATEVSNMSRAQILQQAGTSVLAQANQVPQNVLSLLRHHHHHH

SEQ ID NO: 40 - RNA, 1503 nt, Artificial Sequence
  Synthetic Polynucleotide
auggcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacaacggcgagacaaucgacaucgaccugaagcagaucaacucucagacccugggccucgacacccugaacgugcagaagaaguacgacgucaagagcgaggccgugacaccuagcgccacacugucuacaacagcccuggauggcgccggacucaagacaggcacaggcagcacaacagacaccggcuccaucaaggacggcaagguguacuacaacuccaccagcaagaacuacuacgucgagguggaauucaccgacgccaccgaccagacaaacaaaggcggcuucuacaaagugaacguggccgacgauggggccgugacuaugacagccgccacuaccaaagaggccaccacaccuacaggcaucaccgaagugacccaggugcagaaaccuguggcugccccugcugcuauucaggcccaacugacagcugcccaugugacaggcgccgauacagccgagauggucaagaugagcuacaccgacaagaacggcaagaccauugacggcggcuucggagugaaagugggcgccgacaucuaugccgccaccaagaacaaggauggcagcuucagcaucaauaccaccgaguacaccgauaaggacgggaacaccaagacagcccugaaccagcuuggcggagccgauggaaagaccgagguggugucuaucgauggcaagaccuacaacgccagcaaggccgcuggccacaacuuuaaggcucagccugaacuggccgaagcugccgcugcuaccacagagaauccucuggccaagaucgaugccgcucuggcacaaguggaugcccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugagacaccaucaccaccaccau

SEQ ID NO: 41 - protein, 495 aa, Artificial Sequence
  Synthetic Polypeptide
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANNGETIDIDLKQINSQTLGLDTLNVQKKYDVKSEAVTPSATLSTTALDGAGLKTGTGSTTDTGSIKDGKVYYNSTSKNYYVEVEFTDATDQTNKGGFYKVNVADDGAVTMTAATTKEATTPTGITEVTQVQKPVAAPAAIQAQLTAAHVTGADTAEMVKMSYTDKNGKTIDGGFGVKVGADIYAATKNKDGSFSINTTEYTDKDGNTKTALNQLGGADGKTEVVSIDGKTYNASKAAGHNFKAQPELAEAAAATTENPLAKIDAALAQVDALRSDLGAVQNRFNSAITNLGNTVNNLSSARSRIEDSDYATEVSNMSRAQILQQAGTSVLAQANQVPQNVLSLLR

SEQ ID NO: 42 - RNA, 1485 nt, Artificial Sequence
  Synthetic Polynucleotide
auggcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacaacggcgagacaaucgacaucgaccugaagcagaucaacucucagacccugggccucgacacccugaacgugcagaagaaguacgacgucaagagcgaggccgugacaccuagcgccacacugucuacaacagcccuggauggcgccggacucaagacaggcacaggcagcacaacagacaccggcuccaucaaggacggcaagguguacuacaacuccaccagcaagaacuacuacgucgagguggaauucaccgacgccaccgaccagacaaacaaaggcggcuucuacaaagugaacguggccgacgauggggccgugacuaugacagccgccacuaccaaagaggccaccacaccuacaggcaucaccgaagugacccaggugcagaaaccuguggcugccccugcugcuauucaggcccaacugacagcugcccaugugacaggcgccgauacagccgagauggucaagaugagcuacaccgacaagaacggcaagaccauugacggcggcuucggagugaaagugggcgccgacaucuaugccgccaccaagaacaaggauggcagcuucagcaucaauaccaccgaguacaccgauaaggacgggaacaccaagacagcccugaaccagcuuggcggagccgauggaaagaccgagguggugucuaucgauggcaagaccuacaacgccagcaaggccgcuggccacaacuuuaaggcucagccugaacuggccgaagcugccgcugcuaccacagagaauccucuggccaagaucgaugccgcucuggcacaaguggaugcccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugaga

SEQ ID NO: 43 - protein, 519 aa, Artificial Sequence
  Synthetic Polypeptide
METPAQLLFLLLLWLPDTTAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANNGETIDIDLKQINSQTLGLDTLNVQKKYDVKSEAVTPSATLSTTALDGAGLKTGTGSTTDTGSIKDGKVYYNSTSKNYYVEVEFTDATDQTNKGGFYKVNVADDGAVTMTAATTKEATTPTGITEVTQVQKPVAAPAAIQAQLTAAHVTGADTAEMVKMSYTDKNGKTIDGGFGVKVGADIYAATKNKDGSFSINTTEYTDKDGNTKTALNQLGGADGKTEVVSIDGKTYNASKAAGHNFKAQPELAEAAAATTENPLAKIDAALAQVDALRSDLGAVQNRFNSAITNLGNTVNNLSSARSRIEDSDYATEVSNMSRAQILQQAGTSVLAQANQVPQNVLSLLRHHHHHH

SEQ ID NO: 44 - RNA, 1557 nt, Artificial Sequence
  Synthetic Polynucleotide
auggaaaccccugcucagcugcuguuccugcugcugcuguggcugccugauacaacagcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacaacggcgagacaaucgacaucgaccugaagcagaucaacucucagacccugggccucgacacccugaacgugcagaagaaguacgacgucaagagcgaggccgugacaccuagcgccacacugucuacaacagcccuggauggcgccggacucaagacaggcacaggcagcacaacagacaccggcuccaucaaggacggcaagguguacuacaacuccaccagcaagaacuacuacgucgagguggaauucaccgacgccaccgaccagacaaacaaaggcggcuucuacaaagugaacguggccgacgauggggccgugacuaugacagccgccacuaccaaagaggccaccacaccuacaggcaucaccgaagugacccaggugcagaaaccuguggcugccccugcugcuauucaggcccaacugacagcugcccaugugacaggcgccgauacagccgagauggucaagaugagcuacaccgacaagaacggcaagaccauugacggcggcuucggagugaaagugggcgccgacaucuaugccgccaccaagaacaaggauggcagcuucagcaucaauaccaccgaguacaccgauaaggacgggaacaccaagacagcccugaaccagcuuggcggagccgauggaaagaccgagguggugucuaucgauggcaagaccuacaacgccagcaaggccgcuggccacaacuuuaaggcucagccugaacuggccgaagcugccgcugcuaccacagagaauccucuggccaagaucgaugccgcucuggcacaaguggaugcccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugagacaccaucaccaccaccau

SEQ ID NO: 45 - protein, 513 aa, Artificial Sequence
  Synthetic Polypeptide
METPAQLLFLLLLWLPDTTAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANNGETIDIDLKQINSQTLGLDTLNVQKKYDVKSEAVTPSATLSTTALDGAGLKTGTGSTTDTGSIKDGKVYYNSTSKNYYVEVEFTDATDQTNKGGFYKVNVADDGAVTMTAATTKEATTPTGITEVTQVQKPVAAPAAIQAQLTAAHVTGADTAEMVKMSYTDKNGKTIDGGFGVKVGADIYAATKNKDGSFSINTTEYTDKDGNTKTALNQLGGADGKTEVVSIDGKTYNASKAAGHNFKAQPELAEAAAATTENPLAKIDAALAQVDALRSDLGAVQNRFNSAITNLGNTVNNLSSARSRIEDSDYATEVSNMSRAQILQQAGTSVLAQANQVPQNVLSLLR

SEQ ID NO: 46 - RNA, 1539 nt, Artificial Sequence
  Synthetic Polynucleotide
auggaaaccccugcucagcugcuguuccugcugcugcuguggcugccugauacaacagcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacaacggcgagacaaucgacaucgaccugaagcagaucaacucucagacccugggccucgacacccugaacgugcagaagaaguacgacgucaagagcgaggccgugacaccuagcgccacacugucuacaacagcccuggauggcgccggacucaagacaggcacaggcagcacaacagacaccggcuccaucaaggacggcaagguguacuacaacuccaccagcaagaacuacuacgucgagguggaauucaccgacgccaccgaccagacaaacaaaggcggcuucuacaaagugaacguggccgacgauggggccgugacuaugacagccgccacuaccaaagaggccaccacaccuacaggcaucaccgaagugacccaggugcagaaaccuguggcugccccugcugcuauucaggcccaacugacagcugcccaugugacaggcgccgauacagccgagauggucaagaugagcuacaccgacaagaacggcaagaccauugacggcggcuucggagugaaagugggcgccgacaucuaugccgccaccaagaacaaggauggcagcuucagcaucaauaccaccgaguacaccgauaaggacgggaacaccaagacagcccugaaccagcuuggcggagccgauggaaagaccgagguggugucuaucgauggcaagaccuacaacgccagcaaggccgcuggccacaacuuuaaggcucagccugaacuggccgaagcugccgcugcuaccacagagaauccucuggccaagaucgaugccgcucuggcacaaguggaugcccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugaga

SEQ ID NO: 47 - protein, 501 aa, Artificial Sequence
  Synthetic Polypeptide
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGKVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLRHHHHHH

SEQ ID NO: 48 - RNA, 1503 nt, Artificial Sequence
  Synthetic Polynucleotide
auggcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaagcagaucaacucucagacccugggccucgacacccugaacgugcagcagaaguacaagguuuccgacaccgccgccaccgugacaggcuaugccgauacaacaaucgcccuggacaacagcaccuucaaggccucugccacaggccucggaggcaccgaucagaagauugacggcgaucugaaguucgacgacaccaccggcaaguacuacgccaaagugacagugacaggcggcacaggcaaggacggcuacuacgaaguguccguggacaagaccaacggcaaagugacucuggcuggcggagccaccucuccucuuacugguggacuuccugccaccgccaccgaggacgugaagaaugugcaggucgccaacgccgaucugaccgaagcuaaagccgcucugacagcugcuggcgugaccggaacagccucuguggucaagaugagcuacaccgacaacaacggcaagaccaucgacggcggacuggcagugaaagugggcgacgauuacuacagcgccacacagaacaaggauggcagcaucuccaucaauaccaccaaguacaccgccgacgauggcaccucuaagaccgcucugaacaaacucggcggagccgauggcaagaccgagguggugucuaucggcggcaagacauacgccgccucuaaggccgagggccacaacuuuaaagcccagccugaucuggcugaggccgcugccacuaccacagagaauccucugcagaagaucgacgccgcucuggcacaaguggauacccugagaagcgaucugggcgccgugcagaacagguucaauagcgccaucaccaaccugggcaacaccgugaacaaucugaccagcgccagaagccggaucgaggacagcgauuaugccacagagguguccaacaugagcagagcccagauccugcagcaggccggaacaucuguucuggcucaggccaaucaggugccccagaaugugcugucccugcugagacaccaucaccaccaccau

SEQ ID NO: 49 - protein, 495 aa, Artificial Sequence
  Synthetic Polypeptide
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGKVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 50 - RNA, 1485 nt, Artificial Sequence
  Synthetic Polynucleotide
auggcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaagcagaucaacucucagacccugggccucgacacccugaacgugcagcagaaguacaagguuuccgacaccgccgccaccgugacaggcuaugccgauacaacaaucgcccuggacaacagcaccuucaaggccucugccacaggccucggaggcaccgaucagaagauugacggcgaucugaaguucgacgacaccaccggcaaguacuacgccaaagugacagugacaggcggcacaggcaaggacggcuacuacgaaguguccguggacaagaccaacggcaaagugacucuggcuggcggagccaccucuccucuuacugguggacuuccugccaccgccaccgaggacgugaagaaugugcaggucgccaacgccgaucugaccgaagcuaaagccgcucugacagcugcuggcgugaccggaacagccucuguggucaagaugagcuacaccgacaacaacggcaagaccaucgacggcggacuggcagugaaagugggcgacgauuacuacagcgccacacagaacaaggauggcagcaucuccaucaauaccaccaaguacaccgccgacgauggcaccucuaagaccgcucugaacaaacucggcggagccgauggcaagaccgagguggugucuaucggcggcaagacauacgccgccucuaaggccgagggccacaacuuuaaagcccagccugaucuggcugaggccgcugccacuaccacagagaauccucugcagaagaucgacgccgcucuggcacaaguggauacccugagaagcgaucugggcgccgugcagaacagguucaauagcgccaucaccaaccugggcaacaccgugaacaaucugaccagcgccagaagccggaucgaggacagcgauuaugccacagagguguccaacaugagcagagcccagauccugcagcaggccggaacaucuguucuggcucaggccaaucaggugccccagaaugugcugucccugcugaga

SEQ ID NO: 51 - protein, 519 aa, Artificial Sequence
  Synthetic Polypeptide
METPAQLLFLLLLWLPDTTAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGKVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLRHHHHHH

SEQ ID NO: 52 - RNA, 1557 nt, Artificial Sequence
  Synthetic Polynucleotide
auggaaaccccugcucagcugcuguuccugcugcugcuguggcugccugauacaacagcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaagcagaucaacucucagacccugggccucgacacccugaacgugcagcagaaguacaagguuuccgacaccgccgccaccgugacaggcuaugccgauacaacaaucgcccuggacaacagcaccuucaaggccucugccacaggccucggaggcaccgaucagaagauugacggcgaucugaaguucgacgacaccaccggcaaguacuacgccaaagugacagugacaggcggcacaggcaaggacggcuacuacgaaguguccguggacaagaccaacggcaaagugacucuggcuggcggagccaccucuccucuuacugguggacuuccugccaccgccaccgaggacgugaagaaugugcaggucgccaacgccgaucugaccgaagcuaaagccgcucugacagcugcuggcgugaccggaacagccucuguggucaagaugagcuacaccgacaacaacggcaagaccaucgacggcggacuggcagugaaagugggcgacgauuacuacagcgccacacagaacaaggauggcagcaucuccaucaauaccaccaaguacaccgccgacgauggcaccucuaagaccgcucugaacaaacucggcggagccgauggcaagaccgagguggugucuaucggcggcaagacauacgccgccucuaaggccgagggccacaacuuuaaagcccagccugaucuggcugaggccgcugccacuaccacagagaauccucugcagaagaucgacgccgcucuggcacaaguggauacccugagaagcgaucugggcgccgugcagaacagguucaauagcgccaucaccaaccugggcaacaccgugaacaaucugaccagcgccagaagccggaucgaggacagcgauuaugccacagagguguccaacaugagcagagcccagauccugcagcaggccggaacaucuguucuggcucaggccaaucaggugccccagaaugugcugucccugcugagacaccaucaccaccaccau

SEQ ID NO: 53 - protein, 513 aa, Artificial Sequence
  Synthetic Polypeptide
METPAQLLFLLLLWLPDTTAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGKVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 54 - RNA, 1539 nt, Artificial Sequence
  Synthetic Polynucleotide
auggaaaccccugcucagcugcuguuccugcugcugcuguggcugccugauacaacagcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaagcagaucaacucucagacccugggccucgacacccugaacgugcagcagaaguacaagguuuccgacaccgccgccaccgugacaggcuaugccgauacaacaaucgcccuggacaacagcaccuucaaggccucugccacaggccucggaggcaccgaucagaagauugacggcgaucugaaguucgacgacaccaccggcaaguacuacgccaaagugacagugacaggcggcacaggcaaggacggcuacuacgaaguguccguggacaagaccaacggcaaagugacucuggcuggcggagccaccucuccucuuacugguggacuuccugccaccgccaccgaggacgugaagaaugugcaggucgccaacgccgaucugaccgaagcuaaagccgcucugacagcugcuggcgugaccggaacagccucuguggucaagaugagcuacaccgacaacaacggcaagaccaucgacggcggacuggcagugaaagugggcgacgauuacuacagcgccacacagaacaaggauggcagcaucuccaucaauaccaccaaguacaccgccgacgauggcaccucuaagaccgcucugaacaaacucggcggagccgauggcaagaccgagguggugucuaucggcggcaagacauacgccgccucuaaggccgagggccacaacuuuaaagcccagccugaucuggcugaggccgcugccacuaccacagagaauccucugcagaagaucgacgccgcucuggcacaaguggauacccugagaagcgaucugggcgccgugcagaacagguucaauagcgccaucaccaaccugggcaacaccgugaacaaucugaccagcgccagaagccggaucgaggacagcgauuaugccacagagguguccaacaugagcagagcccagauccugcagcaggccggaacaucuguucuggcucaggccaaucaggugccccagaaugugcugucccugcugaga

SEQ ID NO: 55 - protein, 298 aa, Artificial Sequence
  Synthetic Polypeptide
MKIQEVKRILTRWQPSSFTLYREVFTQYGGSINMHPDIVDYFMKRHNWHFKFFHYKEDDKIKGAYFICNDQNIGILTRRTFPLSSDEILIPMAPDLRCFLPDRTNRLSALHQPQIRNAIWKLARKKQNCLVKETFSSKFEKTRRNEYQRFLKKGGSVKSVADCSSDELTHIFIELFRSRFGNTSSCYPADNLANFFSQLHHLLFGHILYIEGIPCAFDIVLKSESQMNVYFDVSNGAIKNECRPLSPGSILMWLNISRARHYCQERQKKLLFSIGILKPEWEYKRMWSTPYFTGKSIC

SEQ ID NO: 56 - RNA, 894 nt, Artificial Sequence
  Synthetic Polynucleotide
augaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccagcagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucagccgggcucgccacuacugccaggagagacagaagaagcuucucuucucaaucggaaucuuaaagccugagugggaguacaagagaauguggagcaccccuuacuucaccggcaagagcaucugc

SEQ ID NO: 57 - protein, 304 aa, Artificial Sequence
  Synthetic Polypeptide
MKIQEVKRILTRWQPSSFTLYREVFTQYGGSINMHPDIVDYFMKRHNWHFKFFHYKEDDKIKGAYFICNDQNIGILTRRTFPLSSDEILIPMAPDLRCFLPDRTNRLSALHQPQIRNAIWKLARKKQNCLVKETFSSKFEKTRRNEYQRFLKKGGSVKSVADCSSDELTHIFIELFRSRFGNTSSCYPADNLANFFSQLHHLLFGHILYIEGIPCAFDIVLKSESQMNVYFDVSNGAIKNECRPLSPGSILMWLNISRARHYCQERQKKLLFSIGILKPEWEYKRMWSTPYFTGKSICHHHHHH

SEQ ID NO: 58 - RNA, 912 nt, Artificial Sequence
  Synthetic Polynucleotide
augaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccagcagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucagccgggcucgccacuacugccaggagagacagaagaagcuucucuucucaaucggaaucuuaaagccugagugggaguacaagagaauguggagcaccccuuacuucaccggcaagagcaucugccaccaccaucaucaccac

SEQ ID NO: 59 - protein, 317 aa, Artificial Sequence
  Synthetic Polypeptide
METPAQLLLLLLLWLPDTTGKIQEVKRILTRWQPSSFTLYREVFTQYGGSINMHPDIVDYFMKRHNWHFKFFHYKEDDKIKGAYFICNDQNIGILTRRTFPLSSDEILIPMAPDLRCFLPDRTNRLSALHQPQIRNAIWKLARKKQNCLVKETFSSKFEKTRRNEYQRFLKKGGSVKSVADCSSDELTHIFIELFRSRFGNTSSCYPADNLANFFSQLHHLLFGHILYIEGIPCAFDIVLKSESQMNVYFDVSNGAIKNECRPLSPGSILMWLNISRARHYCQERQKKLLFSIGILKPEWEYKRMWSTPYFTGKSIC

SEQ ID NO: 60 - RNA, 951 nt, Artificial Sequence
  Synthetic Polynucleotide
auggagacuccagcccagcuccuccuccuccucuugcucugguugccggacaccaccggcaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccagcagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucagccgggcucgccacuacugccaggagagacagaagaagcuucucuucucaaucggaaucuuaaagccugagugggaguacaagagaauguggagcaccccuuacuucaccggcaagagcaucugc

SEQ ID NO: 61 - protein, 323 aa, Artificial Sequence
  Synthetic Polypeptide
METPAQLLLLLLLWLPDTTGKIQEVKRILTRWQPSSFTLYREVFTQYGGSINMHPDIVDYFMKRHNWHFKFFHYKEDDKIKGAYFICNDQNIGILTRRTFPLSSDEILIPMAPDLRCFLPDRTNRLSALHQPQIRNAIWKLARKKQNCLVKETFSSKFEKTRRNEYQRFLKKGGSVKSVADCSSDELTHIFIELFRSRFGNTSSCYPADNLANFFSQLHHLLFGHILYIEGIPCAFDIVLKSESQMNVYFDVSNGAIKNECRPLSPGSILMWLNISRARHYCQERQKKLLFSIGILKPEWEYKRMWSTPYFTGKSICHHHHHH

SEQ ID NO: 62 - RNA, 969 nt, Artificial Sequence
  Synthetic Polynucleotide
auggagacuccagcccagcuccuccuccuccucuugcucugguugccggacaccaccggcaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccagcagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucagccgggcucgccacuacugccaggagagacagaagaagcuucucuucucaaucggaaucuuaaagccugagugggaguacaagagaauguggagcaccccuuacuucaccggcaagagcaucugccaccaccaucaucaccac

SEQ ID NO: 63 - protein, 298 aa, Artificial Sequence
  Synthetic Polypeptide
MKIQEVKRILTRWQPSSFTLYREVFTQYGGSINMHPDIVDYFMKRHNWHFKFFHYKEDDKIKGAYFICNDQNIGILTRRTFPLSSDEILIPMAPDLRCFLPDRTNRLSALHQPQIRNAIWKLARKKQNCLVKETFSSKFEKTRRNEYQRFLKKGGSVKSVADCSSDELTHIFIELFRSRFGNTASCYPADNLANFFSQLHHLLFGHILYIEGIPCAFDIVLKSESQMNVYFDVSNGAIKNECRPLSPGSILMWLNIARARHYCQERQKKLLFSIGILKPEWEYKRMWSTPYFTGKSIC

SEQ ID NO: 64 - RNA, 894 nt, Artificial Sequence
  Synthetic Polynucleotide
augaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccgccagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucgcccgggcucgccacuacugccaggagagacagaagaagcuucucuucucaaucggaaucuuaaagccugagugggaguacaagagaauguggagcaccccuuacuucaccggcaagagcaucugc

SEQ ID NO: 65 - protein, 304 aa, Artificial Sequence
  Synthetic Polypeptide
MKIQEVKRILTRWQPSSFTLYREVFTQYGGSINMHPDIVDYFMKRHNWHFKFFHYKEDDKIKGAYFICNDQNIGILTRRTFPLSSDEILIPMAPDLRCFLPDRTNRLSALHQPQIRNAIWKLARKKQNCLVKETFSSKFEKTRRNEYQRFLKKGGSVKSVADCSSDELTHIFIELFRSRFGNTASCYPADNLANFFSQLHHLLFGHILYIEGIPCAFDIVLKSESQMNVYFDVSNGAIKNECRPLSPGSILMWLNIARARHYCQERQKKLLFSIGILKPEWEYKRMWSTPYFTGKSICHHHHHH

SEQ ID NO: 66 - RNA, 912 nt, Artificial Sequence
  Synthetic Polynucleotide
augaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccgccagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucgcccgggcucgccacuacugccaggagagacagaagaagcuucucuucucaaucggaaucuuaaagccugagugggaguacaagagaauguggagcaccccuuacuucaccggcaagagcaucugccaccaccaucaucaccac

SEQ ID NO: 67 - protein, 317 aa, Artificial Sequence
  Synthetic Polypeptide
METPAQLLLLLLLWLPDTTGKIQEVKRILTRWQPSSFTLYREVFTQYGGSINMHPDIVDYFMKRHNWHFKFFHYKEDDKIKGAYFICNDQNIGILTRRTFPLSSDEILIPMAPDLRCFLPDRTNRLSALHQPQIRNAIWKLARKKQNCLVKETFSSKFEKTRRNEYQRFLKKGGSVKSVADCSSDELTHIFIELFRSRFGNTASCYPADNLANFFSQLHHLLFGHILYIEGIPCAFDIVLKSESQMNVYFDVSNGAIKNECRPLSPGSILMWLNIARARHYCQERQKKLLFSIGILKPEWEYKRMWSTPYFTGKSIC

SEQ ID NO: 68 - RNA, 951 nt, Artificial Sequence
  Synthetic Polynucleotide
auggagacuccagcccagcuccuccuccuccucuugcucugguugccggacaccaccggcaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccgccagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucgcccgggcucgccacuacugccaggagagacagaagaagcuucucuucucaaucggaaucuuaaagccugagugggaguacaagagaauguggagcaccccuuacuucaccggcaagagcaucugc

SEQ ID NO: 69 - protein, 323 aa, Artificial Sequence
  Synthetic Polypeptide
METPAQLLLLLLLWLPDTTGKIQEVKRILTRWQPSSFTLYREVFTQYGGSINMHPDIVDYFMKRHNWHFKFFHYKEDDKIKGAYFICNDQNIGILTRRTFPLSSDEILIPMAPDLRCFLPDRTNRLSALHQPQIRNAIWKLARKKQNCLVKETFSSKFEKTRRNEYQRFLKKGGSVKSVADCSSDELTHIFIELFRSRFGNTASCYPADNLANFFSQLHHLLFGHILYIEGIPCAFDIVLKSESQMNVYFDVSNGAIKNECRPLSPGSILMWLNIARARHYCQERQKKLLFSIGILKPEWEYKRMWSTPYFTGKSICHHHHHH

SEQ ID NO: 70 - RNA, 969 nt, Artificial Sequence
  Synthetic Polynucleotide
auggagacuccagcccagcuccuccuccuccucuugcucugguugccggacaccaccggcaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccgccagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucgcccgggcucgccacuacugccaggagagacagaagaagcuucucuucucaaucggaaucuuaaagccugagugggaguacaagagaauguggagcaccccuuacuucaccggcaagagcaucugccaccaccaucaucaccac

SEQ ID NO: 71 - protein, 742 aa, Artificial Sequence
  Synthetic Polypeptide
MGLQTTKWPSHGAFFLKSWLIISLGLYSQVSKLLAAESTDDNGETIVVESTAEQVLKQQPGVSIITRDDIQKNPPVNDLADIIRKMPGVNLTSNSASGTRGNNRQIDIRGMGPENTLVLIDGVPVTSRNSVRYSWRGERDTRGDTNWVPPEMVERIEMIRGPAAARYGSGAAGGVVNIITKRPTNDWHGSLSLYTNYPESSKEGDTRRGNFSLSGPLAGDTLTMRLYGNLNRTDADSWDINSSAGTKNAAGREGVTNKDINSVFSWKMTPQQILDFEAGYSRQGNIYAGDTQNSNSNAVTKSLAQSGRETNRLYRQNYGLTHNGIWGWGQSRLGFYYEKTDNTRMNEGLSGGGEGRITNDQTFTTNRLTSYRTSGEVNVPVIWLFEQTLTVGAEWNRDELNDPSSTSLTVKDSNIAGIPGSAANRSSKNKSEISALYVEDNIEPMAGTNIIPGLRFDYLSESGSNFSPSLNLSQELGEFVKVKAGIARAFKAPNLYQTSEGYLLYSKGNGCPKDITSGGCYLVGNKNLDPEISINKEIGLEFTVDDYHASVTYFRNDYQNKIVAGDQIIGRSASGAYVLQWQNGGKALIEGIEASMAVPLMPDRLNWNTNATYMITSEQKDTGNPLSIIPKYTVNTFLDWTITNALSANVNWTLYGKQKPRTHAESRSEETKGLSGKALGAYSLVGANVNYDINKNLRLNVGISNIFDKQIYRSAEGANTYNEPGRAYYAGVTASFHHHHHH

SEQ ID NO: 72 - RNA, 2226 nt, Artificial Sequence
  Synthetic Polynucleotide
augggccugcagaccacaaaguggccuucucacggcgcauucuuucugaaguccuggcugaucaucucccugggccuguacagccagguguccaaacugcuggccgccgagagcaccgacgauaacggcgagacaaucgugguggaaagcaccgccgaacaggugcugaaacagcagccuggcguguccaucaucacccgggacgacauccagaagaacccuccagucaacgaccuggccgacaucaucagaaagaugcccggcgugaaccugaccagcaauagcgccucuggcacccggggcaacaacagacagaucgacaucagaggcaugggccccgagaauacccuggugcugauugauggcgugcccgugaccagcagaaacagcgugcgguauucuuggagaggcgagagagacaccagaggcgacaccaauugggugccaccugagaugguggaacggaucgagaugauuagaggccccgcugccgccagauauggaucuggugcugcuggcggcguggucaauaucaucaccaagaggcccaccaacgacuggcacggcagccugagccuguauacaaacuaccccgagagcagcaaagagggcgauaccagaagaggcaacuuuagccugucuggcccucuggccggcgauacccugacaaugagacuguacggcaaccugaaccggaccgacgccgauagcugggacaucaauagcagcgccggcaccaagaaugccgccggaagagaaggcgugaccaacaaggacaucaacagcguguucagcuggaagaugaccccucagcagauccuggauuucgaggccggcuauagcagacagggcaacaucuaugccggcgacacccagaacagcaacagcaacgccgugaccaagucucuggcccagucuggcagagagacaaacaggcuguaccggcagaacuacggccugacacacaauggcaucuggggcuggggacagucuaggcugggcuucuacuacgagaagaccgacaacacccggaugaacgagggacuuucuggcggcggagagggcagaaucaccaacgaucagaccuucaccaccaaccggcugaccagcuacagaaccagcggcgaagugaacgugccagugaucuggcuguucgagcagacccugacagugggcgccgaguggaauagagaugagcugaacgaccccagcuccaccagccugaccgugaaggacucuaauaucgcuggcaucccuggcagcgccgccaacagaagcagcaagaacaagagcgagaucagcgcccuguacgucgaggacaacaucgaaccuauggccggcacaaacaucauccccggccugagauucgacuaccugagcgagagcggcagcaacuucagccccagccugaaucugucucaagagcugggcgaguucgugaaagugaaggccggaaucgccagagccuucaaggccccuaaucuguaccagaccagcgagggcuaccugcuguacuccaaaggcaacggcugccccaaggauaucacaagcggcggcuguuaccucgugggcaacaagaaccuggauccugagaucagcaucaacaaagagaucggccuggaauucaccguggacgacuaccacgccagcgugaccuacuuccggaacgauuaccagaacaagaucguggccggggaccagaucaucggcagaagugcuagcggagccuacgugcugcaauggcagaauggcggaaaggcccugaucgagggaaucgaggcuucuauggccgugccacugaugcccgacagacugaacuggaacaccaacgccaccuacaugaucaccagcgagcagaaggacaccggcaauccucugagcaucaucccuaaguauaccgugaacaccuuccuggacuggaccaucacaaacgcccugagcgccaacgugaacuggacccuguauggcaagcagaagccacggacacacgccgaguccagaagcgaggaaacaaagggccugagcggcaaagcccugggcgccuauucucuugugggcgccaaugugaauuacgacauuaacaagaaucugcggcugaacgugggcaucagcaacaucuucgacaagcagaucuacagaagcgccgagggcgccaacaccuacaaugaaccuggcagagccuacuaugcuggcgugaccgccagcuuccaucaccaccaucaucau

SEQ ID NO: 73 - protein, 736 aa, Artificial Sequence
  Synthetic Polypeptide
MGLQTTKWPSHGAFFLKSWLIISLGLYSQVSKLLAAESTDDNGETIVVESTAEQVLKQQPGVSIITRDDIQKNPPVNDLADIIRKMPGVNLTSNSASGTRGNNRQIDIRGMGPENTLVLIDGVPVTSRNSVRYSWRGERDTRGDTNWVPPEMVERIEMIRGPAAARYGSGAAGGVVNIITKRPTNDWHGSLSLYTNYPESSKEGDTRRGNFSLSGPLAGDTLTMRLYGNLNRTDADSWDINSSAGTKNAAGREGVTNKDINSVFSWKMTPQQILDFEAGYSRQGNIYAGDTQNSNSNAVTKSLAQSGRETNRLYRQNYGLTHNGIWGWGQSRLGFYYEKTDNTRMNEGLSGGGEGRITNDQTFTTNRLTSYRTSGEVNVPVIWLFEQTLTVGAEWNRDELNDPSSTSLTVKDSNIAGIPGSAANRSSKNKSEISALYVEDNIEPMAGTNIIPGLRFDYLSESGSNFSPSLNLSQELGEFVKVKAGIARAFKAPNLYQTSEGYLLYSKGNGCPKDITSGGCYLVGNKNLDPEISINKEIGLEFTVDDYHASVTYFRNDYQNKIVAGDQIIGRSASGAYVLQWQNGGKALIEGIEASMAVPLMPDRLNWNTNATYMITSEQKDTGNPLSIIPKYTVNTFLDWTITNALSANVNWTLYGKQKPRTHAESRSEETKGLSGKALGAYSLVGANVNYDINKNLRLNVGISNIFDKQIYRSAEGANTYNEPGRAYYAGVTASF

SEQ ID NO: 74 - RNA, 2208 nt, Artificial Sequence
  Synthetic Polynucleotide
augggccugcagaccacaaaguggccuucucacggcgcauucuuucugaaguccuggcugaucaucucccugggccuguacagccagguguccaaacugcuggccgccgagagcaccgacgauaacggcgagacaaucgugguggaaagcaccgccgaacaggugcugaaacagcagccuggcguguccaucaucacccgggacgacauccagaagaacccuccagucaacgaccuggccgacaucaucagaaagaugcccggcgugaaccugaccagcaauagcgccucuggcacccggggcaacaacagacagaucgacaucagaggcaugggccccgagaauacccuggugcugauugauggcgugcccgugaccagcagaaacagcgugcgguauucuuggagaggcgagagagacaccagaggcgacaccaauugggugccaccugagaugguggaacggaucgagaugauuagaggccccgcugccgccagauauggaucuggugcugcuggcggcguggucaauaucaucaccaagaggcccaccaacgacuggcacggcagccugagccuguauacaaacuaccccgagagcagcaaagagggcgauaccagaagaggcaacuuuagccugucuggcccucuggccggcgauacccugacaaugagacuguacggcaaccugaaccggaccgacgccgauagcugggacaucaauagcagcgccggcaccaagaaugccgccggaagagaaggcgugaccaacaaggacaucaacagcguguucagcuggaagaugaccccucagcagauccuggauuucgaggccggcuauagcagacagggcaacaucuaugccggcgacacccagaacagcaacagcaacgccgugaccaagucucuggcccagucuggcagagagacaaacaggcuguaccggcagaacuacggccugacacacaauggcaucuggggcuggggacagucuaggcugggcuucuacuacgagaagaccgacaacacccggaugaacgagggacuuucuggcggcggagagggcagaaucaccaacgaucagaccuucaccaccaaccggcugaccagcuacagaaccagcggcgaagugaacgugccagugaucuggcuguucgagcagacccugacagugggcgccgaguggaauagagaugagcugaacgaccccagcuccaccagccugaccgugaaggacucuaauaucgcuggcaucccuggcagcgccgccaacagaagcagcaagaacaagagcgagaucagcgcccuguacgucgaggacaacaucgaaccuauggccggcacaaacaucauccccggccugagauucgacuaccugagcgagagcggcagcaacuucagccccagccugaaucugucucaagagcugggcgaguucgugaaagugaaggccggaaucgccagagccuucaaggccccuaaucuguaccagaccagcgagggcuaccugcuguacuccaaaggcaacggcugccccaaggauaucacaagcggcggcuguuaccucgugggcaacaagaaccuggauccugagaucagcaucaacaaagagaucggccuggaauucaccguggacgacuaccacgccagcgugaccuacuuccggaacgauuaccagaacaagaucguggccggggaccagaucaucggcagaagugcuagcggagccuacgugcugcaauggcagaauggcggaaaggcccugaucgagggaaucgaggcuucuauggccgugccacugaugcccgacagacugaacuggaacaccaacgccaccuacaugaucaccagcgagcagaaggacaccggcaauccucugagcaucaucccuaaguauaccgugaacaccuuccuggacuggaccaucacaaacgcccugagcgccaacgugaacuggacccuguauggcaagcagaagccacggacacacgccgaguccagaagcgaggaaacaaagggccugagcggcaaagcccugggcgccuauucucuugugggcgccaaugugaauuacgacauuaacaagaaucugcggcugaacgugggcaucagcaacaucuucgacaagcagaucuacagaagcgccgagggcgccaacaccuacaaugaaccuggcagagccuacuaugcuggcgugaccgccagcuuc

SEQ ID NO: 75 - protein, 742 aa, Artificial Sequence
  Synthetic Polypeptide
MGLQTTKWPSHGAFFLKSWLIISLGLYSQVSKLLAAESTDDNGETIVVESTAEQVLKQQPGVSIITRDDIQKNPPVNDLADIIRKMPGVNLASNSASGTRGNNRQIDIRGMGPENTLVLIDGVPVTSRNSVRYSWRGERDTRGDTNWVPPEMVERIEMIRGPAAARYGSGAAGGVVNIITKRPTNDWHGSLSLYTNYPESSKEGDTRRGNFALSGPLAGDTLTMRLYGNLNRADADSWDINSSAGTKNAAGREGVTNKDINSVFSWKMTPQQILDFEAGYSRQGNIYAGDTQNSNSNAVTKSLAQSGRETNRLYRQNYGLTHNGIWGWGQSRLGFYYEKTDNTRMNEGLSGGGEGRITNDQTFTTNRLTSYRTSGEVNVPVIWLFEQTLTVGAEWNRDELNDPSSTSLTVKDSNIAGIPGSAANRASKNKSEISALYVEDNIEPMAGTNIIPGLRFDYLSESGSNFSPSLNLAQELGEFVKVKAGIARAFKAPNLYQTSEGYLLYSKGNGCPKDITSGGCYLVGNKNLDPEISINKEIGLEFTVDDYHASVTYFRNDYQNKIVAGDQIIGRSASGAYVLQWQNGGKALIEGIEASMAVPLMPDRLNWNTNAAYMITSEQKDTGNPLSIIPKYTVNTFLDWTITNALSANVNWTLYGKQKPRTHAESRSEETKGLSGKALGAYSLVGANVNYDINKNLRLNVGISNIFDKQIYRSAEGANTYNEPGRAYYAGVTASFHHHHHH

SEQ ID NO: 76 - RNA, 2226 nt, Artificial Sequence
  Synthetic Polynucleotide
augggccugcagaccacaaaguggccuucucacggcgcauucuuucugaaguccuggcugaucaucucccugggccuguacagccagguguccaaacugcuggccgccgagagcaccgacgauaacggcgagacaaucgugguggaaagcaccgccgaacaggugcugaaacagcagccuggcguguccaucaucacccgggacgacauccagaagaacccuccagucaacgaccuggccgacaucaucagaaagaugcccggcgugaaccuggccagcaauagcgccucuggcacccggggcaacaacagacagaucgacaucagaggcaugggccccgagaauacccuggugcugauugauggcgugcccgugaccagcagaaacagcgugcgguauucuuggagaggcgagagagacaccagaggcgacaccaauugggugccaccugagaugguggaacggaucgagaugauuagaggccccgcugccgccagauauggaucuggugcugcuggcggcguggucaauaucaucaccaagaggcccaccaacgacuggcacggcagccugagccuguauacaaacuaccccgagagcagcaaagagggcgauaccagaagaggcaacuuugcccugucuggcccucuggccggcgauacccugacaaugagacuguacggcaaccugaaccgggccgacgccgauagcugggacaucaauagcagcgccggcaccaagaaugccgccggaagagaaggcgugaccaacaaggacaucaacagcguguucagcuggaagaugaccccucagcagauccuggauuucgaggccggcuauagcagacagggcaacaucuaugccggcgacacccagaacagcaacagcaacgccgugaccaagucucuggcccagucuggcagagagacaaacaggcuguaccggcagaacuacggccugacacacaauggcaucuggggcuggggacagucuaggcugggcuucuacuacgagaagaccgacaacacccggaugaacgagggacuuucuggcggcggagagggcagaaucaccaacgaucagaccuucaccaccaaccggcugaccagcuacagaaccagcggcgaagugaacgugccagugaucuggcuguucgagcagacccugacagugggcgccgaguggaauagagaugagcugaacgaccccagcuccaccagccugaccgugaaggacucuaauaucgcuggcaucccuggcagcgccgccaacagagccagcaagaacaagagcgagaucagcgcccuguacgucgaggacaacaucgaaccuauggccggcacaaacaucauccccggccugagauucgacuaccugagcgagagcggcagcaacuucagccccagccugaaucuggcucaagagcugggcgaguucgugaaagugaaggccggaaucgccagagccuucaaggccccuaaucuguaccagaccagcgagggcuaccugcuguacuccaaaggcaacggcugccccaaggauaucacaagcggcggcuguuaccucgugggcaacaagaaccuggauccugagaucagcaucaacaaagagaucggccuggaauucaccguggacgacuaccacgccagcgugaccuacuuccggaacgauuaccagaacaagaucguggccggggaccagaucaucggcagaagugcuagcggagccuacgugcugcaauggcagaauggcggaaaggcccugaucgagggaaucgaggcuucuauggccgugccacugaugcccgacagacugaacuggaacaccaacgccgccuacaugaucaccagcgagcagaaggacaccggcaauccucugagcaucaucccuaaguauaccgugaacaccuuccuggacuggaccaucacaaacgcccugagcgccaacgugaacuggacccuguauggcaagcagaagccacggacacacgccgaguccagaagcgaggaaacaaagggccugagcggcaaagcccugggcgccuauucucuugugggcgccaaugugaauuacgacauuaacaagaaucugcggcugaacgugggcaucagcaacaucuucgacaagcagaucuacagaagcgccgagggcgccaacaccuacaaugaaccuggcagagccuacuaugcuggcgugaccgccagcuuccaucaccaccaucaucau

SEQ ID NO: 77 - protein, 736 aa, Artificial Sequence
  Synthetic Polypeptide
MGLQTTKWPSHGAFFLKSWLIISLGLYSQVSKLLAAESTDDNGETIVVESTAEQVLKQQPGVSIITRDDIQKNPPVNDLADIIRKMPGVNLASNSASGTRGNNRQIDIRGMGPENTLVLIDGVPVTSRNSVRYSWRGERDTRGDTNWVPPEMVERIEMIRGPAAARYGSGAAGGVVNIITKRPTNDWHGSLSLYTNYPESSKEGDTRRGNFALSGPLAGDTLTMRLYGNLNRADADSWDINSSAGTKNAAGREGVTNKDINSVFSWKMTPQQILDFEAGYSRQGNIYAGDTQNSNSNAVTKSLAQSGRETNRLYRQNYGLTHNGIWGWGQSRLGFYYEKTDNTRMNEGLSGGGEGRITNDQTFTTNRLTSYRTSGEVNVPVIWLFEQTLTVGAEWNRDELNDPSSTSLTVKDSNIAGIPGSAANRASKNKSEISALYVEDNIEPMAGTNIIPGLRFDYLSESGSNFSPSLNLAQELGEFVKVKAGIARAFKAPNLYQTSEGYLLYSKGNGCPKDITSGGCYLVGNKNLDPEISINKEIGLEFTVDDYHASVTYFRNDYQNKIVAGDQIIGRSASGAYVLQWQNGGKALIEGIEASMAVPLMPDRLNWNTNAAYMITSEQKDTGNPLSIIPKYTVNTFLDWTITNALSANVNWTLYGKQKPRTHAESRSEETKGLSGKALGAYSLVGANVNYDINKNLRLNVGISNIFDKQIYRSAEGANTYNEPGRAYYAGVTASF

SEQ ID NO: 78 - RNA, 2208 nt, Artificial Sequence
  Synthetic Polynucleotide
augggccugcagaccacaaaguggccuucucacggcgcauucuuucugaaguccuggcugaucaucucccugggccuguacagccagguguccaaacugcuggccgccgagagcaccgacgauaacggcgagacaaucgugguggaaagcaccgccgaacaggugcugaaacagcagccuggcguguccaucaucacccgggacgacauccagaagaacccuccagucaacgaccuggccgacaucaucagaaagaugcccggcgugaaccuggccagcaauagcgccucuggcacccggggcaacaacagacagaucgacaucagaggcaugggccccgagaauacccuggugcugauugauggcgugcccgugaccagcagaaacagcgugcgguauucuuggagaggcgagagagacaccagaggcgacaccaauugggugccaccugagaugguggaacggaucgagaugauuagaggccccgcugccgccagauauggaucuggugcugcuggcggcguggucaauaucaucaccaagaggcccaccaacgacuggcacggcagccugagccuguauacaaacuaccccgagagcagcaaagagggcgauaccagaagaggcaacuuugcccugucuggcccucuggccggcgauacccugacaaugagacuguacggcaaccugaaccgggccgacgccgauagcugggacaucaauagcagcgccggcaccaagaaugccgccggaagagaaggcgugaccaacaaggacaucaacagcguguucagcuggaagaugaccccucagcagauccuggauuucgaggccggcuauagcagacagggcaacaucuaugccggcgacacccagaacagcaacagcaacgccgugaccaagucucuggcccagucuggcagagagacaaacaggcuguaccggcagaacuacggccugacacacaauggcaucuggggcuggggacagucuaggcugggcuucuacuacgagaagaccgacaacacccggaugaacgagggacuuucuggcggcggagagggcagaaucaccaacgaucagaccuucaccaccaaccggcugaccagcuacagaaccagcggcgaagugaacgugccagugaucuggcuguucgagcagacccugacagugggcgccgaguggaauagagaugagcugaacgaccccagcuccaccagccugaccgugaaggacucuaauaucgcuggcaucccuggcagcgccgccaacagagccagcaagaacaagagcgagaucagcgcccuguacgucgaggacaacaucgaaccuauggccggcacaaacaucauccccggccugagauucgacuaccugagcgagagcggcagcaacuucagccccagccugaaucuggcucaagagcugggcgaguucgugaaagugaaggccggaaucgccagagccuucaaggccccuaaucuguaccagaccagcgagggcuaccugcuguacuccaaaggcaacggcugccccaaggauaucacaagcggcggcuguuaccucgugggcaacaagaaccuggauccugagaucagcaucaacaaagagaucggccuggaauucaccguggacgacuaccacgccagcgugaccuacuuccggaacgauuaccagaacaagaucguggccggggaccagaucaucggcagaagugcuagcggagccuacgugcugcaauggcagaauggcggaaaggcccugaucgagggaaucgaggcuucuauggccgugccacugaugcccgacagacugaacuggaacaccaacgccgccuacaugaucaccagcgagcagaaggacaccggcaauccucugagcaucaucccuaaguauaccgugaacaccuuccuggacuggaccaucacaaacgcccugagcgccaacgugaacuggacccuguauggcaagcagaagccacggacacacgccgaguccagaagcgaggaaacaaagggccugagcggcaaagcccugggcgccuauucucuugugggcgccaaugugaauuacgacauuaacaagaaucugcggcugaacgugggcaucagcaacaucuucgacaagcagaucuacagaagcgccgagggcgccaacaccuacaaugaaccuggcagagccuacuaugcuggcgugaccgccagcuuc

SEQ ID NO: 79 - protein, 679 aa, Artificial Sequence
  Synthetic Polypeptide
MGLQTTKWPSHGAFFLKSWLIISLGLYSQVSKLLAATDDGETMVVTASAIEQNLKDAPASISVITQQDLQRRPVQNLKDVLKEVPGVQLTNEGDNRKGVSIRGLDSSYTLILIDGKRVNSRNAVFRHNDFDLNWIPVDAIERIEVVRGPMSSLYGSDALGGVVNIITKKIGQKWHGSVTVDSTIQEHRDRGDTYNGQFFTSGPLIDGVLGMKAYGSLAKREKDEQQSSATTATGETPRIEGFTSRDGNVEFAWTPNENHDVTAGYGFDRQDRDSDSLDKNRLERQNYALSHNGRWDLGNSELKFYGEKVENKNPGNSSPITSESNSIDGKYVLPLASVNQFLTFGGEWRHDKLSDAVSLTGGSSTKTSASQYALFLEDEWRIFEPLALTTGIRMDDHETYGDHWSPRAYLVYNATDTLTVKGGWATAFKAPSLLQLSPDWATNSCRGGCRIVGSPDLKPETSESWELGLYYRGEEGILEGVEASVTTFRNDVDNRISISRTPDVNAAPGYSNFVGFETNSRGQRVPVFRYYNVNKARIQGVETELKVPFNEAWKLSLNYTYNDGRDVSNGGNKPLSDLPFHTANGTLDWKPAQLEDWSFYVSGNYTGRKRADSATAKTPGGYVVWDTGAAWQATKNVKLRAGVLNVGDKDLKRDDYGYTEDGRRYFMAVDYRFHHHHHH

SEQ ID NO: 80 - RNA, 2037 nt, Artificial Sequence
  Synthetic Polynucleotide
augggccugcagaccacaaaguggccuucucacggcgcauucuuucugaaguccuggcugaucaucucccugggccuguacagccagguguccaaacugcuggccgccaccgaugauggcgagacaauggugguuacagccagcgccaucgagcagaaccugaaagaugccccugccagcaucagcgugaucacccagcaggaucugcagagaaggcccgugcagaaucugaaggacgugcugaaagaggugcccggcguccagcugacaaacgagggcgauaauagaaagggcguguccaucagaggccuggacagcagcuacacccugauccugaucgacggcaagagagugaacagccggaacgccguguuccggcacaacgacuucgaccugaacuggauccccguggaugccaucgagaggaucgaaguugugcggggaccuaugagcagccuguacggaucugaugcccucggcggcguggucaacaucaucaccaagaagaucggccagaaauggcacggcagcgugaccguggauagcaccauccaagagcacagagacagaggcgacaccuacaacggccaguucuucacaagcggcccucugauugauggcgugcugggcaugaaggccuacggaucucuggccaagagagagaaggacgagcagcagagcagcgccacaacagccacaggcgagacaccuagaaucgagggcuucaccagcagagauggcaacguggaauucgccuggacacccaacgagaaccacgaugugacagccggcuacggcuucgacagacaggacagagauagcgacagccuggacaagaaccggcuggaaagacagaacuacgcccugagccacaacggcagaugggaccugggaaacagcgagcugaaguucuacggcgagaaggucgagaacaagaaccccggcaacagcagcccuaucaccagcgagagcaacagcaucgauggcaaauacgugcugccccuggccuccgugaaucaguuccugacauuuggcggagaguggcggcacgacaagcugucugaugccguuucucugaccggcggcagcagcacaaagacaagcgccucucaguacgcccuguuccuggaagaugaguggcggaucuucgagccccuggcucugacaacaggcaucagaauggacgaccacgagacauacggcgaccacugguccccaagagccuaccugguguacaacgccaccgacacacugaccgugaaaggcggaugggccacagccuuuaaggcuccuagucugcugcagcugagccccgauugggccaccaauucuuguagaggcggcugcagaaucgugggcagcccugaucugaagcccgagacaucugagucuugggagcugggacuguacuacaggggcgaagagggaauccuggaaggcguggaagccagcgugacaaccuucagaaacgacguggacaaccggaucagcaucuccagaacacccgacgugaacgccgcuccuggcuacucuaauuucgugggcuucgagacaaacagcagaggccagagggugcccguguuucgguacuacaacgugaacaaggcccggauccagggcgucgagacagagcugaaggugcccuuuaacgaagccuggaagcugagccugaacuacacauacaacgacggccgggacguguccaacggcggaaacaaaccucugagcgaccugccuuuccacaccgccaauggcacccuggauuggaagccugcucagcuggaagauuggagcuucuacguguccggcaacuacaccggcagaaagagagccgauagcgccaccgcuaagacaccaggcggauacgucgugugggauacaggugcugcuuggcaggccaccaagaacgugaaacugagagccggcgugcugaacgugggcgacaaagaccugaagagggacgacuacggcuacaccgaggacggcagacgguacuucauggccguggacuaccgguuccaccaccaccaucaccau

SEQ ID NO: 81 - protein, 673 aa, Artificial Sequence
  Synthetic Polypeptide
MGLQTTKWPSHGAFFLKSWLIISLGLYSQVSKLLAATDDGETMVVTASAIEQNLKDAPASISVITQQDLQRRPVQNLKDVLKEVPGVQLTNEGDNRKGVSIRGLDSSYTLILIDGKRVNSRNAVFRHNDFDLNWIPVDAIERIEVVRGPMSSLYGSDALGGVVNIITKKIGQKWHGSVTVDSTIQEHRDRGDTYNGQFFTSGPLIDGVLGMKAYGSLAKREKDEQQSSATTATGETPRIEGFTSRDGNVEFAWTPNENHDVTAGYGFDRQDRDSDSLDKNRLERQNYALSHNGRWDLGNSELKFYGEKVENKNPGNSSPITSESNSIDGKYVLPLASVNQFLTFGGEWRHDKLSDAVSLTGGSSTKTSASQYALFLEDEWRIFEPLALTTGIRMDDHETYGDHWSPRAYLVYNATDTLTVKGGWATAFKAPSLLQLSPDWATNSCRGGCRIVGSPDLKPETSESWELGLYYRGEEGILEGVEASVTTFRNDVDNRISISRTPDVNAAPGYSNFVGFETNSRGQRVPVFRYYNVNKARIQGVETELKVPFNEAWKLSLNYTYNDGRDVSNGGNKPLSDLPFHTANGTLDWKPAQLEDWSFYVSGNYTGRKRADSATAKTPGGYVVWDTGAAWQATKNVKLRAGVLNVGDKDLKRDDYGYTEDGRRYFMAVDYRF

SEQ ID NO: 82 - RNA, 2019 nt, Artificial Sequence
  Synthetic Polynucleotide
augggccugcagaccacaaaguggccuucucacggcgcauucuuucugaaguccuggcugaucaucucccugggccuguacagccagguguccaaacugcuggccgccaccgaugauggcgagacaauggugguuacagccagcgccaucgagcagaaccugaaagaugccccugccagcaucagcgugaucacccagcaggaucugcagagaaggcccgugcagaaucugaaggacgugcugaaagaggugcccggcguccagcugacaaacgagggcgauaauagaaagggcguguccaucagaggccuggacagcagcuacacccugauccugaucgacggcaagagagugaacagccggaacgccguguuccggcacaacgacuucgaccugaacuggauccccguggaugccaucgagaggaucgaaguugugcggggaccuaugagcagccuguacggaucugaugcccucggcggcguggucaacaucaucaccaagaagaucggccagaaauggcacggcagcgugaccguggauagcaccauccaagagcacagagacagaggcgacaccuacaacggccaguucuucacaagcggcccucugauugauggcgugcugggcaugaaggccuacggaucucuggccaagagagagaaggacgagcagcagagcagcgccacaacagccacaggcgagacaccuagaaucgagggcuucaccagcagagauggcaacguggaauucgccuggacacccaacgagaaccacgaugugacagccggcuacggcuucgacagacaggacagagauagcgacagccuggacaagaaccggcuggaaagacagaacuacgcccugagccacaacggcagaugggaccugggaaacagcgagcugaaguucuacggcgagaaggucgagaacaagaaccccggcaacagcagcccuaucaccagcgagagcaacagcaucgauggcaaauacgugcugccccuggccuccgugaaucaguuccugacauuuggcggagaguggcggcacgacaagcugucugaugccguuucucugaccggcggcagcagcacaaagacaagcgccucucaguacgcccuguuccuggaagaugaguggcggaucuucgagccccuggcucugacaacaggcaucagaauggacgaccacgagacauacggcgaccacugguccccaagagccuaccugguguacaacgccaccgacacacugaccgugaaaggcggaugggccacagccuuuaaggcuccuagucugcugcagcugagccccgauugggccaccaauucuuguagaggcggcugcagaaucgugggcagcccugaucugaagcccgagacaucugagucuugggagcugggacuguacuacaggggcgaagagggaauccuggaaggcguggaagccagcgugacaaccuucagaaacgacguggacaaccggaucagcaucuccagaacacccgacgugaacgccgcuccuggcuacucuaauuucgugggcuucgagacaaacagcagaggccagagggugcccguguuucgguacuacaacgugaacaaggcccggauccagggcgucgagacagagcugaaggugcccuuuaacgaagccuggaagcugagccugaacuacacauacaacgacggccgggacguguccaacggcggaaacaaaccucugagcgaccugccuuuccacaccgccaauggcacccuggauuggaagccugcucagcuggaagauuggagcuucuacguguccggcaacuacaccggcagaaagagagccgauagcgccaccgcuaagacaccaggcggauacgucgugugggauacaggugcugcuuggcaggccaccaagaacgugaaacugagagccggcgugcugaacgugggcgacaaagaccugaagagggacgacuacggcuacaccgaggacggcagacgguacuucauggccguggacuaccgguuc

SEQ ID NO: 83 - protein, 679 aa, Artificial Sequence
  Synthetic Polypeptide
MGLQTTKWPSHGAFFLKSWLIISLGLYSQVSKLLAATDDGETMVVTASAIEQNLKDAPASISVITQQDLQRRPVQNLKDVLKEVPGVQLTNEGDNRKGVSIRGLDSSYTLILIDGKRVNSRNAVFRHNDFDLNWIPVDAIERIEVVRGPMSSLYGSDALGGVVNIITKKIGQKWHGSVTVDSTIQEHRDRGDTYNGQFFTSGPLIDGVLGMKAYGSLAKREKDEQQSSATTATGETPRIEGFTSRDGNVEFAWTPNENHDVTAGYGFDRQDRDSDSLDKNRLERQNYALSHNGRWDLGNSELKFYGEKVENKNPGNSSPITSESNSIDGKYVLPLASVNQFLTFGGEWRHDKLSDAVSLTGGSSTKTSASQYALFLEDEWRIFEPLALTTGIRMDDHETYGDHWSPRAYLVYNAADTLTVKGGWATAFKAPSLLQLSPDWATNSCRGGCRIVGSPDLKPETSESWELGLYYRGEEGILEGVEASVTTFRNDVDNRISISRTPDVNAAPGYSNFVGFETNSRGQRVPVFRYYNVNKARIQGVETELKVPFNEAWKLSLNYAYNDGRDVSNGGNKPLSDLPFHTANGALDWKPAQLEDWSFYVSGNYAGRKRADSATAKTPGGYVVWDTGAAWQATKNVKLRAGVLNVGDKDLKRDDYGYTEDGRRYFMAVDYRFHHHHHH

SEQ ID NO: 84 - RNA, 2037 nt, Artificial Sequence
  Synthetic Polynucleotide
augggccugcagaccacaaaguggccuucucacggcgcauucuuucugaaguccuggcugaucaucucccugggccuguacagccagguguccaaacugcuggccgccaccgaugauggcgagacaauggugguuacagccagcgccaucgagcagaaccugaaagaugccccugccagcaucagcgugaucacccagcaggaucugcagagaaggcccgugcagaaucugaaggacgugcugaaagaggugcccggcguccagcugacaaacgagggcgauaauagaaagggcguguccaucagaggccuggacagcagcuacacccugauccugaucgacggcaagagagugaacagccggaacgccguguuccggcacaacgacuucgaccugaacuggauccccguggaugccaucgagaggaucgaaguugugcggggaccuaugagcagccuguacggaucugaugcccucggcggcguggucaacaucaucaccaagaagaucggccagaaauggcacggcagcgugaccguggauagcaccauccaagagcacagagacagaggcgacaccuacaacggccaguucuucacaagcggcccucugauugauggcgugcugggcaugaaggccuacggaucucuggccaagagagagaaggacgagcagcagagcagcgccacaacagccacaggcgagacaccuagaaucgagggcuucaccagcagagauggcaacguggaauucgccuggacacccaacgagaaccacgaugugacagccggcuacggcuucgacagacaggacagagauagcgacagccuggacaagaaccggcuggaaagacagaacuacgcccugagccacaacggcagaugggaccugggaaacagcgagcugaaguucuacggcgagaaggucgagaacaagaaccccggcaacagcagcccuaucaccagcgagagcaacagcaucgauggcaaauacgugcugccccuggccuccgugaaucaguuccugacauuuggcggagaguggcggcacgacaagcugucugaugccguuucucugaccggcggcagcagcacaaagacaagcgccucucaguacgcccuguuccuggaagaugaguggcggaucuucgagccccuggcucugacaacaggcaucagaauggacgaccacgagacauacggcgaccacugguccccaagagccuaccugguguacaacgccgccgacacacugaccgugaaaggcggaugggccacagccuuuaaggcuccuagucugcugcagcugagccccgauugggccaccaauucuuguagaggcggcugcagaaucgugggcagcccugaucugaagcccgagacaucugagucuugggagcugggacuguacuacaggggcgaagagggaauccuggaaggcguggaagccagcgugacaaccuucagaaacgacguggacaaccggaucagcaucuccagaacacccgacgugaacgccgcuccuggcuacucuaauuucgugggcuucgagacaaacagcagaggccagagggugcccguguuucgguacuacaacgugaacaaggcccggauccagggcgucgagacagagcugaaggugcccuuuaacgaagccuggaagcugagccugaacuacgcauacaacgacggccgggacguguccaacggcggaaacaaaccucugagcgaccugccuuuccacaccgccaauggcgcccuggauuggaagccugcucagcuggaagauuggagcuucuacguguccggcaacuacgccggcagaaagagagccgauagcgccaccgcuaagacaccaggcggauacgucgugugggauacaggugcugcuuggcaggccaccaagaacgugaaacugagagccggcgugcugaacgugggcgacaaagaccugaagagggacgacuacggcuacaccgaggacggcagacgguacuucauggccguggacuaccgguuccaccaccaccaucaccau

SEQ ID NO: 85 - protein, 673 aa, Artificial Sequence
  Synthetic Polypeptide
MGLQTTKWPSHGAFFLKSWLIISLGLYSQVSKLLAATDDGETMVVTASAIEQNLKDAPASISVITQQDLQRRPVQNLKDVLKEVPGVQLTNEGDNRKGVSIRGLDSSYTLILIDGKRVNSRNAVFRHNDFDLNWIPVDAIERIEVVRGPMSSLYGSDALGGVVNIITKKIGQKWHGSVTVDSTIQEHRDRGDTYNGQFFTSGPLIDGVLGMKAYGSLAKREKDEQQSSATTATGETPRIEGFTSRDGNVEFAWTPNENHDVTAGYGFDRQDRDSDSLDKNRLERQNYALSHNGRWDLGNSELKFYGEKVENKNPGNSSPITSESNSIDGKYVLPLASVNQFLTFGGEWRHDKLSDAVSLTGGSSTKTSASQYALFLEDEWRIFEPLALTTGIRMDDHETYGDHWSPRAYLVYNAADTLTVKGGWATAFKAPSLLQLSPDWATNSCRGGCRIVGSPDLKPETSESWELGLYYRGEEGILEGVEASVTTFRNDVDNRISISRTPDVNAAPGYSNFVGFETNSRGQRVPVFRYYNVNKARIQGVETELKVPFNEAWKLSLNYAYNDGRDVSNGGNKPLSDLPFHTANGALDWKPAQLEDWSFYVSGNYAGRKRADSATAKTPGGYVVWDTGAAWQATKNVKLRAGVLNVGDKDLKRDDYGYTEDGRRYFMAVDYRF

SEQ ID NO: 86 - RNA, 2019 nt, Artificial Sequence
  Synthetic Polynucleotide
augggccugcagaccacaaaguggccuucucacggcgcauucuuucugaaguccuggcugaucaucucccugggccuguacagccagguguccaaacugcuggccgccaccgaugauggcgagacaauggugguuacagccagcgccaucgagcagaaccugaaagaugccccugccagcaucagcgugaucacccagcaggaucugcagagaaggcccgugcagaaucugaaggacgugcugaaagaggugcccggcguccagcugacaaacgagggcgauaauagaaagggcguguccaucagaggccuggacagcagcuacacccugauccugaucgacggcaagagagugaacagccggaacgccguguuccggcacaacgacuucgaccugaacuggauccccguggaugccaucgagaggaucgaaguugugcggggaccuaugagcagccuguacggaucugaugcccucggcggcguggucaacaucaucaccaagaagaucggccagaaauggcacggcagcgugaccguggauagcaccauccaagagcacagagacagaggcgacaccuacaacggccaguucuucacaagcggcccucugauugauggcgugcugggcaugaaggccuacggaucucuggccaagagagagaaggacgagcagcagagcagcgccacaacagccacaggcgagacaccuagaaucgagggcuucaccagcagagauggcaacguggaauucgccuggacacccaacgagaaccacgaugugacagccggcuacggcuucgacagacaggacagagauagcgacagccuggacaagaaccggcuggaaagacagaacuacgcccugagccacaacggcagaugggaccugggaaacagcgagcugaaguucuacggcgagaaggucgagaacaagaaccccggcaacagcagcccuaucaccagcgagagcaacagcaucgauggcaaauacgugcugccccuggccuccgugaaucaguuccugacauuuggcggagaguggcggcacgacaagcugucugaugccguuucucugaccggcggcagcagcacaaagacaagcgccucucaguacgcccuguuccuggaagaugaguggcggaucuucgagccccuggcucugacaacaggcaucagaauggacgaccacgagacauacggcgaccacugguccccaagagccuaccugguguacaacgccgccgacacacugaccgugaaaggcggaugggccacagccuuuaaggcuccuagucugcugcagcugagccccgauugggccaccaauucuuguagaggcggcugcagaaucgugggcagcccugaucugaagcccgagacaucugagucuugggagcugggacuguacuacaggggcgaagagggaauccuggaaggcguggaagccagcgugacaaccuucagaaacgacguggacaaccggaucagcaucuccagaacacccgacgugaacgccgcuccuggcuacucuaauuucgugggcuucgagacaaacagcagaggccagagggugcccguguuucgguacuacaacgugaacaaggcccggauccagggcgucgagacagagcugaaggugcccuuuaacgaagccuggaagcugagccugaacuacgcauacaacgacggccgggacguguccaacggcggaaacaaaccucugagcgaccugccuuuccacaccgccaauggcgcccuggauuggaagccugcucagcuggaagauuggagcuucuacguguccggcaacuacgccggcagaaagagagccgauagcgccaccgcuaagacaccaggcggauacgucgugugggauacaggugcugcuuggcaggccaccaagaacgugaaacugagagccggcgugcugaacgugggcgacaaagaccugaagagggacgacuacggcuacaccgaggacggcagacgguacuucauggccguggacuaccgguuc

SEQ ID NO: 87 - protein, 225 aa, Artificial Sequence
  Synthetic Polypeptide
MDSKGSSQKGSRLLLLLVVSNLLLPQGVVGTSHHDSHGLHRVGSGSAMQIYEGKLTAEGLRFGIVASRFNHALVDRLVEGAIDAIVRHGGREEDITLVRVPGSWEIPVAAGELARKENISAVIAIGVLIRGATPHFDYIASEVSKGLADLSLELRKPITFGVITADTLEQAIERAGTKHGNKGWEAALSAIEMANLFKSLRGGLVPRGSHHHHHHSAWSHPQFEK

SEQ ID NO: 88 - RNA, 675 nt, Artificial Sequence
  Synthetic Polynucleotide
auggauagcaagggcagcagccagaagggcuccagacugcugcugcuucuggugguguccaaccugcuucugccucaaggcguugugggcacgagccaccacgacagccacggguugcacagggugggaagcggcagcgccaugcagaucuacgagggaaagcugaccgccgagggccugagauuuggaaucguggccagccgguucaaucacgcccugguggauagacugguggaaggcgccaucgaugccauugucagacacggcggcagagaagaggacaucacccucguuagagugcccggcucuugggagauuccuguggcugcuggcgagcuggcccggaaagagaauaucucugccguuaucgccaucggcgugcugaucagaggcgccacaccucacuucgacuauaucgccagcgagguguccaaaggccuggccgaucugagccuggaacugagaaagcccaucaccuucggcgugaucaccgcugacacacuggaacaggccauugagagagccggcaccaagcacggcaacaaaggaugggaagccgcucuguccgccaucgagauggccaaccuguucaagagccugagaggcggccuggugccuagaggaucucaccaccaccaucaccacagcgcuuggagccauccucaguucgagaag

SEQ ID NO: 89 - protein, 752 aa, Artificial Sequence
  Synthetic Polypeptide
METPAQLLFLLLLWLPDTTGEEKTDSAALTNEDTIVVTAAQQNLQAPGVSTITADEIRKNPPARDVSEIIRTMPGVNLTGNSTSGQRGNNRQIDIRGMGPENTLILIDGKPVTSRNSVRLGWRGERDTRGDTAWVPPEMIERIEVLRGPAAARYGNGAAGGVVNIITKKGGSEWHGSWNTYFNAPEHKDEGATKRTNFSLNGPLGGDFSFRLYGNLDKTQADARNINQGHQSERTGSYADTLPAGREGVINKDINGVVRWDFAPLQSLELEAGYSRQGNLYAGDTQNTNTNQLVKDNYGKETNRLYRQNYSLTWNGGWDNGVTTSNWVQYEHTRNSRMPEGLAGGTEGIFDPKASQKYADADLNDVTLHSEVSLPFDLLVNQNLTLGTEWAQQRMKDQLSNSQTFMGGNIPGYSSTNRSPYSKAEIFSLFAENNMELTDSTMLTPGIRFDHHSIVGDNWSPSLNLSQGLGDDFTLKMGIARAYKAPSLYQTNPNYILYSKGQGCYATGAGTGIGCYMMGNDDLKAETSINKEIGLEFKRDGWLAGVTWFRNDYRNKIEAGTVPLQRINNGKTDVYQWENVPKAVVEGLEGTLNVPVSDTVNWTNNVTYMLQSKNKETGERLSIIPQYTLNSTLSWQVRQDVSLQSTFTWYGKQEPKKYDYQGNPVTGTDKQAVSPYSIVGLSATWDVTKNVSLTGGVDNLFDKRLWREGNAQTVRDTQTGAYMAGAGAYTYNEPGRTWYMSINTHFHHHHHH

SEQ ID NO: 90 - RNA, 2256 nt, Artificial Sequence
  Synthetic Polynucleotide
auggagacaccugcccagcugcuguuccugcugcugcuguggcugccugacaccaccggggaggagaagaccgacagcgcagcccugaccaacgaggacaccaucguggugaccgccgcucagcagaaccugcaggcgcccggcgugagcaccaucaccgccgacgagaucagaaagaacccuccugccagagacgugagcgagaucaucagaaccaugccuggcgugaaccugaccggcaacagcaccagcggccagagaggcaacaacagacagaucgacaucagaggcaugggcccugagaacacccugauccugaucgacggcaagcccgugaccagcaggaauagcgugagacugggcuggagaggggagcgcgacacaaggggcgauaccgccugggugccuccugagaugaucgagagaaucgaggugcugagaggcccugccgccgccagauacgggaacggagccgccggcggcguggugaacaucaucaccaagaagggcggcagcgaguggcacggcagcuggaacaccuacuucaacgccccugagcacaaggacgagggcgccaccaagagaaccaacuucagccugaacggcccucugggcggcgacuucagcuucagacuguacggcaaccuggacaagacccaggccgacgccagaaacaucaaccagggccaccagagcgagagaaccggcagcuacgccgacacccugccugccggcagagagggcgugaucaacaaggacaucaacggcgugguccgcugggauuucgccccacugcagagccuggagcuggaggccggcuacagcagacaggggaaccuguacgccggcgauacccagaacaccaacaccaaccagcuggugaaggacaacuacggcaaggagacaaauagacuguaccgccagaacuacagccugaccuggaacggcggcugggacaacggggugaccaccagcaacugggugcaguacgagcacaccagaaacagcagaaugcccgaggggcuggccgguggcacugaaggcaucuucgacccuaaagccagccagaaauacgcugacgccgaucugaacgacgugacccugcacagcgaggugagccugccuuucgaccugcucgucaaccagaaccugacccugggcaccgagugggcccagcagagaaugaaggaccagcugagcaacagccagaccuucaugggcggaaacauaccaggcuauagcagcaccaacagaagcccuuacagcaaggccgagaucuucucccuguucgccgagaacaacauggagcugaccgacagcaccaugcugaccccuggcaucagauucgaccaucacagcaucgugggcgacaacuggagccccagccugaaccugagccaaggccugggcgacgacuucacccugaagaugggcaucgccagagccuacaaggccccuagccuguaccagaccaacccuaacuacauccuguacagcaagggccagggcuguuacgcaaccggcgccggcacaggcaucggcugcuacaugaugggcaacgacgaccugaaggccgaaacuagcaucaacaaggagaucggccuggaguucaagagggacggcuggcucgccggagugaccugguuuagaaacgacuacagaaacaagaucgaagccggcaccguaccucuucagcggaucaacaacggcaagaccgacguguaccagugggagaacgugccuaaggccgugguggagggccucgagggcacccucaacgugccugugagcgacaccgugaacuggaccaacaacgugaccuacaugcugcagagcaagaacaaggaaaccggcgagagacugagcaucaucccucaguacacccugaacagcacccugagcuggcaggugagacaggacgugucccugcaguccaccuuuacgugguacggcaagcaggagccuaagaaguacgacuaccagggcaacccugugaccggcaccgacaagcaggccgucagcccauauuccaucgugggacucagcgccaccugggacgugaccaagaacgugagccugaccggagggguugacaaccuguucgacaagagacuguggagagagggcaacgcccagaccgugagggacacucagaccggcgccuacauggccggcgcuggggccuacaccuacaacgagcccggucggaccugguacauguccaucaacacccacuuccaccaucaccaucaccac

SEQ ID NO: 91 - protein, 746 aa, Artificial Sequence
  Synthetic Polypeptide
METPAQLLFLLLLWLPDTTGEEKTDSAALTNEDTIVVTAAQQNLQAPGVSTITADEIRKNPPARDVSEIIRTMPGVNLTGNSTSGQRGNNRQIDIRGMGPENTLILIDGKPVTSRNSVRLGWRGERDTRGDTAWVPPEMIERIEVLRGPAAARYGNGAAGGVVNIITKKGGSEWHGSWNTYFNAPEHKDEGATKRTNFSLNGPLGGDFSFRLYGNLDKTQADARNINQGHQSERTGSYADTLPAGREGVINKDINGVVRWDFAPLQSLELEAGYSRQGNLYAGDTQNTNTNQLVKDNYGKETNRLYRQNYSLTWNGGWDNGVTTSNWVQYEHTRNSRMPEGLAGGTEGIFDPKASQKYADADLNDVTLHSEVSLPFDLLVNQNLTLGTEWAQQRMKDQLSNSQTFMGGNIPGYSSTNRSPYSKAEIFSLFAENNMELTDSTMLTPGIRFDHHSIVGDNWSPSLNLSQGLGDDFTLKMGIARAYKAPSLYQTNPNYILYSKGQGCYATGAGTGIGCYMMGNDDLKAETSINKEIGLEFKRDGWLAGVTWFRNDYRNKIEAGTVPLQRINNGKTDVYQWENVPKAVVEGLEGTLNVPVSDTVNWTNNVTYMLQSKNKETGERLSIIPQYTLNSTLSWQVRQDVSLQSTFTWYGKQEPKKYDYQGNPVTGTDKQAVSPYSIVGLSATWDVTKNVSLTGGVDNLFDKRLWREGNAQTVRDTQTGAYMAGAGAYTYNEPGRTWYMSINTHF

SEQ ID NO: 92 - RNA, 2238 nt, Artificial Sequence
  Synthetic Polynucleotide
auggagacaccugcccagcugcuguuccugcugcugcuguggcugccugacaccaccggggaggagaagaccgacagcgcagcccugaccaacgaggacaccaucguggugaccgccgcucagcagaaccugcaggcgcccggcgugagcaccaucaccgccgacgagaucagaaagaacccuccugccagagacgugagcgagaucaucagaaccaugccuggcgugaaccugaccggcaacagcaccagcggccagagaggcaacaacagacagaucgacaucagaggcaugggcccugagaacacccugauccugaucgacggcaagcccgugaccagcaggaauagcgugagacugggcuggagaggggagcgcgacacaaggggcgauaccgccugggugccuccugagaugaucgagagaaucgaggugcugagaggcccugccgccgccagauacgggaacggagccgccggcggcguggugaacaucaucaccaagaagggcggcagcgaguggcacggcagcuggaacaccuacuucaacgccccugagcacaaggacgagggcgccaccaagagaaccaacuucagccugaacggcccucugggcggcgacuucagcuucagacuguacggcaaccuggacaagacccaggccgacgccagaaacaucaaccagggccaccagagcgagagaaccggcagcuacgccgacacccugccugccggcagagagggcgugaucaacaaggacaucaacggcgugguccgcugggauuucgccccacugcagagccuggagcuggaggccggcuacagcagacaggggaaccuguacgccggcgauacccagaacaccaacaccaaccagcuggugaaggacaacuacggcaaggagacaaauagacuguaccgccagaacuacagccugaccuggaacggcggcugggacaacggggugaccaccagcaacugggugcaguacgagcacaccagaaacagcagaaugcccgaggggcuggccgguggcacugaaggcaucuucgacccuaaagccagccagaaauacgcugacgccgaucugaacgacgugacccugcacagcgaggugagccugccuuucgaccugcucgucaaccagaaccugacccugggcaccgagugggcccagcagagaaugaaggaccagcugagcaacagccagaccuucaugggcggaaacauaccaggcuauagcagcaccaacagaagcccuuacagcaaggccgagaucuucucccuguucgccgagaacaacauggagcugaccgacagcaccaugcugaccccuggcaucagauucgaccaucacagcaucgugggcgacaacuggagccccagccugaaccugagccaaggccugggcgacgacuucacccugaagaugggcaucgccagagccuacaaggccccuagccuguaccagaccaacccuaacuacauccuguacagcaagggccagggcuguuacgcaaccggcgccggcacaggcaucggcugcuacaugaugggcaacgacgaccugaaggccgaaacuagcaucaacaaggagaucggccuggaguucaagagggacggcuggcucgccggagugaccugguuuagaaacgacuacagaaacaagaucgaagccggcaccguaccucuucagcggaucaacaacggcaagaccgacguguaccagugggagaacgugccuaaggccgugguggagggccucgagggcacccucaacgugccugugagcgacaccgugaacuggaccaacaacgugaccuacaugcugcagagcaagaacaaggaaaccggcgagagacugagcaucaucccucaguacacccugaacagcacccugagcuggcaggugagacaggacgugucccugcaguccaccuuuacgugguacggcaagcaggagccuaagaaguacgacuaccagggcaacccugugaccggcaccgacaagcaggccgucagcccauauuccaucgugggacucagcgccaccugggacgugaccaagaacgugagccugaccggagggguugacaaccuguucgacaagagacuguggagagagggcaacgcccagaccgugagggacacucagaccggcgccuacauggccggcgcuggggccuacaccuacaacgagcccggucggaccugguacauguccaucaacacccacuuc

SEQ ID NO: 93 - protein, 752 aa, Artificial Sequence
  Synthetic Polypeptide
METPAQLLFLLLLWLPDTTGEEKTDSAALTNEDTIVVTAAQQNLQAPGVSTITADEIRKNPPARDVSEIIRTMPGVNLAGNSASGQRGNNRQIDIRGMGPENTLILIDGKPVTSRNSVRLGWRGERDTRGDTAWVPPEMIERIEVLRGPAAARYGNGAAGGVVNIITKKGGSEWHGSWNTYFNAPEHKDEGATKRTNFALNGPLGGDFSFRLYGNLDKTQADARNINQGHQSERTGSYADTLPAGREGVINKDINGVVRWDFAPLQSLELEAGYSRQGNLYAGDTQNTNTNQLVKDNYGKETNRLYRQNYALTWNGGWDNGVTTSNWVQYEHTRNSRMPEGLAGGTEGIFDPKASQKYADADLNDVTLHSEVSLPFDLLVNQNLALGTEWAQQRMKDQLSNSQTFMGGNIPGYSSTNRSPYSKAEIFSLFAENNMELTDSTMLTPGIRFDHHSIVGDNWSPSLNLSQGLGDDFTLKMGIARAYKAPSLYQTNPNYILYSKGQGCYATGAGTGIGCYMMGNDDLKAETSINKEIGLEFKRDGWLAGVTWFRNDYRNKIEAGTVPLQRINNGKTDVYQWENVPKAVVEGLEGTLNVPVSDTVNWANNVAYMLQSKNKETGERLSIIPQYTLNSALSWQVRQDVSLQSTFTWYGKQEPKKYDYQGNPVTGTDKQAVSPYSIVGLSATWDVTKNVSLTGGVDNLFDKRLWREGNAQTVRDTQTGAYMAGAGAYTYNEPGRTWYMSINTHFHHHHHH

SEQ ID NO: 94 - RNA, 2256 nt, Artificial Sequence
  Synthetic Polynucleotide
auggagacaccugcccagcugcuguuccugcugcugcuguggcugccugacaccaccggggaggagaagaccgacagcgcagcccugaccaacgaggacaccaucguggugaccgccgcucagcagaaccugcaggcgcccggcgugagcaccaucaccgccgacgagaucagaaagaacccuccugccagagacgugagcgagaucaucagaaccaugccuggcgugaaccuggccggcaacagcgccagcggccagagaggcaacaacagacagaucgacaucagaggcaugggcccugagaacacccugauccugaucgacggcaagcccgugaccagcaggaauagcgugagacugggcuggagaggggagcgcgacacaaggggcgauaccgccugggugccuccugagaugaucgagagaaucgaggugcugagaggcccugccgccgccagauacgggaacggagccgccggcggcguggugaacaucaucaccaagaagggcggcagcgaguggcacggcagcuggaacaccuacuucaacgccccugagcacaaggacgagggcgccaccaagagaaccaacuucgcacugaacggcccucugggcggcgacuucagcuucagacuguacggcaaccuggacaagacccaggccgacgccagaaacaucaaccagggccaccagagcgagagaaccggcagcuacgccgacacccugccugccggcagagagggcgugaucaacaaggacaucaacggcgugguccgcugggauuucgccccacugcagagccuggagcuggaggccggcuacagcagacaggggaaccuguacgccggcgauacccagaacaccaacaccaaccagcuggugaaggacaacuacggcaaggagacaaauagacuguaccgccagaacuacgcacugaccuggaacggcggcugggacaacggggugaccaccagcaacugggugcaguacgagcacaccagaaacagcagaaugcccgaggggcuggccgguggcacugaaggcaucuucgacccuaaagccagccagaaauacgcugacgccgaucugaacgacgugacccugcacagcgaggugagccugccuuucgaccugcucgucaaccagaaccuggcccugggcaccgagugggcccagcagagaaugaaggaccagcugagcaacagccagaccuucaugggcggaaacauaccaggcuauagcagcaccaacagaagcccuuacagcaaggccgagaucuucucccuguucgccgagaacaacauggagcugaccgacagcaccaugcugaccccuggcaucagauucgaccaucacagcaucgugggcgacaacuggagccccagccugaaccugagccaaggccugggcgacgacuucacccugaagaugggcaucgccagagccuacaaggccccuagccuguaccagaccaacccuaacuacauccuguacagcaagggccagggcuguuacgcaaccggcgccggcacaggcaucggcugcuacaugaugggcaacgacgaccugaaggccgaaacuagcaucaacaaggagaucggccuggaguucaagagggacggcuggcucgccggagugaccugguuuagaaacgacuacagaaacaagaucgaagccggcaccguaccucuucagcggaucaacaacggcaagaccgacguguaccagugggagaacgugccuaaggccgugguggagggccucgagggcacccucaacgugccugugagcgacaccgugaacugggccaacaacguggccuacaugcugcagagcaagaacaaggaaaccggcgagagacugagcaucaucccucaguacacccugaacagcgcccugagcuggcaggugagacaggacgugucccugcaguccaccuuuacgugguacggcaagcaggagccuaagaaguacgacuaccagggcaacccugugaccggcaccgacaagcaggccgucagcccauauuccaucgugggacucagcgccaccugggacgugaccaagaacgugagccugaccggagggguugacaaccuguucgacaagagacuguggagagagggcaacgcccagaccgugagggacacucagaccggcgccuacauggccggcgcuggggccuacaccuacaacgagcccggucggaccugguacauguccaucaacacccacuuccaccaucaccaucaccac

SEQ ID NO: 95 - protein, 746 aa, Artificial Sequence
  Synthetic Polypeptide
METPAQLLFLLLLWLPDTTGEEKTDSAALTNEDTIVVTAAQQNLQAPGVSTITADEIRKNPPARDVSEIIRTMPGVNLAGNSASGQRGNNRQIDIRGMGPENTLILIDGKPVTSRNSVRLGWRGERDTRGDTAWVPPEMIERIEVLRGPAAARYGNGAAGGVVNIITKKGGSEWHGSWNTYFNAPEHKDEGATKRTNFALNGPLGGDFSFRLYGNLDKTQADARNINQGHQSERTGSYADTLPAGREGVINKDINGVVRWDFAPLQSLELEAGYSRQGNLYAGDTQNTNTNQLVKDNYGKETNRLYRQNYALTWNGGWDNGVTTSNWVQYEHTRNSRMPEGLAGGTEGIFDPKASQKYADADLNDVTLHSEVSLPFDLLVNQNLALGTEWAQQRMKDQLSNSQTFMGGNIPGYSSTNRSPYSKAEIFSLFAENNMELTDSTMLTPGIRFDHHSIVGDNWSPSLNLSQGLGDDFTLKMGIARAYKAPSLYQTNPNYILYSKGQGCYATGAGTGIGCYMMGNDDLKAETSINKEIGLEFKRDGWLAGVTWFRNDYRNKIEAGTVPLQRINNGKTDVYQWENVPKAVVEGLEGTLNVPVSDTVNWANNVAYMLQSKNKETGERLSIIPQYTLNSALSWQVRQDVSLQSTFTWYGKQEPKKYDYQGNPVTGTDKQAVSPYSIVGLSATWDVTKNVSLTGGVDNLFDKRLWREGNAQTVRDTQTGAYMAGAGAYTYNEPGRTWYMSINTHF

SEQ ID NO: 96 - RNA, 2238 nt, Artificial Sequence
  Synthetic Polynucleotide
auggagacaccugcccagcugcuguuccugcugcugcuguggcugccugacaccaccggggaggagaagaccgacagcgcagcccugaccaacgaggacaccaucguggugaccgccgcucagcagaaccugcaggcgcccggcgugagcaccaucaccgccgacgagaucagaaagaacccuccugccagagacgugagcgagaucaucagaaccaugccuggcgugaaccuggccggcaacagcgccagcggccagagaggcaacaacagacagaucgacaucagaggcaugggcccugagaacacccugauccugaucgacggcaagcccgugaccagcaggaauagcgugagacugggcuggagaggggagcgcgacacaaggggcgauaccgccugggugccuccugagaugaucgagagaaucgaggugcugagaggcccugccgccgccagauacgggaacggagccgccggcggcguggugaacaucaucaccaagaagggcggcagcgaguggcacggcagcuggaacaccuacuucaacgccccugagcacaaggacgagggcgccaccaagagaaccaacuucgcacugaacggcccucugggcggcgacuucagcuucagacuguacggcaaccuggacaagacccaggccgacgccagaaacaucaaccagggccaccagagcgagagaaccggcagcuacgccgacacccugccugccggcagagagggcgugaucaacaaggacaucaacggcgugguccgcugggauuucgccccacugcagagccuggagcuggaggccggcuacagcagacaggggaaccuguacgccggcgauacccagaacaccaacaccaaccagcuggugaaggacaacuacggcaaggagacaaauagacuguaccgccagaacuacgcacugaccuggaacggcggcugggacaacggggugaccaccagcaacugggugcaguacgagcacaccagaaacagcagaaugcccgaggggcuggccgguggcacugaaggcaucuucgacccuaaagccagccagaaauacgcugacgccgaucugaacgacgugacccugcacagcgaggugagccugccuuucgaccugcucgucaaccagaaccuggcccugggcaccgagugggcccagcagagaaugaaggaccagcugagcaacagccagaccuucaugggcggaaacauaccaggcuauagcagcaccaacagaagcccuuacagcaaggccgagaucuucucccuguucgccgagaacaacauggagcugaccgacagcaccaugcugaccccuggcaucagauucgaccaucacagcaucgugggcgacaacuggagccccagccugaaccugagccaaggccugggcgacgacuucacccugaagaugggcaucgccagagccuacaaggccccuagccuguaccagaccaacccuaacuacauccuguacagcaagggccagggcuguuacgcaaccggcgccggcacaggcaucggcugcuacaugaugggcaacgacgaccugaaggccgaaacuagcaucaacaaggagaucggccuggaguucaagagggacggcuggcucgccggagugaccugguuuagaaacgacuacagaaacaagaucgaagccggcaccguaccucuucagcggaucaacaacggcaagaccgacguguaccagugggagaacgugccuaaggccgugguggagggccucgagggcacccucaacgugccugugagcgacaccgugaacugggccaacaacguggccuacaugcugcagagcaagaacaaggaaaccggcgagagacugagcaucaucccucaguacacccugaacagcgcccugagcuggcaggugagacaggacgugucccugcaguccaccuuuacgugguacggcaagcaggagccuaagaaguacgacuaccagggcaacccugugaccggcaccgacaagcaggccgucagcccauauuccaucgugggacucagcgccaccugggacgugaccaagaacgugagccugaccggagggguugacaaccuguucgacaagagacuguggagagagggcaacgcccagaccgugagggacacucagaccggcgccuacauggccggcgcuggggccuacaccuacaacgagcccggucggaccugguacauguccaucaacacccacuuc

SEQ ID NO: 97 - protein, 267 aa, Artificial Sequence
  Synthetic Polypeptide
METPAQLLFLLLLWLPDTTGNISDYKVMTWNLQGSSASTESKWNVNVRQLLSGTAGVDILMVQEAGAVPTSAVPTGRHIQPFGVGIPIDEYTWNLGTTSRQDIRYIYHSAIDVGARRVNLAIVSRQRADNVYVLRPTTVASRPVIGIGLGNDVFLTAHALASGGPDAAAIVRVTINFFRQPQMRHLSWFLAGDFNRSPDRLENDLMTEHLERVVAVLAPTEPTQIGGGILDYGVIVDRAPYSQRVEALRNPQLASDHYPVAFLARSC

SEQ ID NO: 98 - RNA, 801 nt, Artificial Sequence
  Synthetic Polynucleotide
auggagacuccugcccagcugcuguuccugcugcugcuguggcugccugacaccaccggcaacaucagcgacuacaaggugaugaccuggaaccugcagggaagcuccgccagcaccgagagcaaguggaacgugaacgugagacagcuccugagcggcaccgccggcgucgacauccugauggugcaggaggccggagccgucccuaccagcgccgugccuaccggcagacacauccagccuuucggcgugggcaucccuaucgacgaguacaccuggaaucucggcaccaccagcagacaggacaucagauacaucuaccacagcgccaucgacgugggcgccagaagagugaaccuggccaucgugagcagacagagagccgacaacguguacgugcugaggccuaccaccguggccagcagaccugugaucggcaucggccugggcaacgacguguuccugaccgcccacgcucuggccuccgguggccccgacgcugccgccaucgugagagugaccaucaacuucuucagacagccucagaugagacaccugagcugguuccuggccggcgacuucaacagaagcccugacagacuggagaacgaccugaugaccgagcaccuggagagagugguggccgugcuggccccuaccgagccuacccagaucggcggcggcauccuggacuacggcgugaucguggacagagccccuuacagccagagaguggaggcccugagaaacccucagcuggccagcgaccacuacccuguggccuuccuggccagaagcugc

SEQ ID NO: 99 - protein, 267 aa, Artificial Sequence
  Synthetic Polypeptide
METPAQLLFLLLLWLPDTTGNIADYKVMTWNLQGSSASTESKWNVNVRQLLSGTAGVDILMVQEAGAVPTSAVPTGRHIQPFGVGIPIDEYTWNLGTTSRQDIRYIYHSAIDVGARRVNLAIVSRQRADNVYVLRPTTVASRPVIGIGLGNDVFLTAHALASGGPDAAAIVRVTINFFRQPQMRHLSWFLAGDFNRSPDRLENDLMTEHLERVVAVLAPTEPTQIGGGILDYGVIVDRAPYSQRVEALRNPQLASDHYPVAFLARSC

SEQ ID NO: 100 - RNA, 801 nt, Artificial Sequence
  Synthetic Polynucleotide
auggagacuccugcccagcugcuguuccugcugcugcuguggcugccugacaccaccggcaacaucgcagacuacaaggugaugaccuggaaccugcagggaagcuccgccagcaccgagagcaaguggaacgugaacgugagacagcuccugagcggcaccgccggcgucgacauccugauggugcaggaggccggagccgucccuaccagcgccgugccuaccggcagacacauccagccuuucggcgugggcaucccuaucgacgaguacaccuggaaucucggcaccaccagcagacaggacaucagauacaucuaccacagcgccaucgacgugggcgccagaagagugaaccuggccaucgugagcagacagagagccgacaacguguacgugcugaggccuaccaccguggccagcagaccugugaucggcaucggccugggcaacgacguguuccugaccgcccacgcucuggccuccgguggccccgacgcugccgccaucgugagagugaccaucaacuucuucagacagccucagaugagacaccugagcugguuccuggccggcgacuucaacagaagcccugacagacuggagaacgaccugaugaccgagcaccuggagagagugguggccgugcuggccccuaccgagccuacccagaucggcggcggcauccuggacuacggcgugaucguggacagagccccuuacagccagagaguggaggcccugagaaacccucagcuggccagcgaccacuacccuguggccuuccuggccagaagcugc

SEQ ID NO: 101 - protein, 273 aa, Artificial Sequence
  Synthetic Polypeptide
METPAQLLFLLLLWLPDTTGNIADYKVMTWNLQGSSASTESKWNVNVRQLLSGTAGVDILMVQEAGAVPTSAVPTGRHIQPFGVGIPIDEYTWNLGTTSRQDIRYIYHSAIDVGARRVNLAIVSRQRADNVYVLRPTTVASRPVIGIGLGNDVFLTAHALASGGPDAAAIVRVTINFFRQPQMRHLSWFLAGDFNRSPDRLENDLMTEHLERVVAVLAPTEPTQIGGGILDYGVIVDRAPYSQRVEALRNPQLASDHYPVAFLARSCHHHHHH

SEQ ID NO: 102 - RNA, 819 nt, Artificial Sequence
  Synthetic Polynucleotide
auggagacuccugcccagcugcuguuccugcugcugcuguggcugccugacaccaccggcaacaucgcagacuacaaggugaugaccuggaaccugcagggaagcuccgccagcaccgagagcaaguggaacgugaacgugagacagcuccugagcggcaccgccggcgucgacauccugauggugcaggaggccggagccgucccuaccagcgccgugccuaccggcagacacauccagccuuucggcgugggcaucccuaucgacgaguacaccuggaaucucggcaccaccagcagacaggacaucagauacaucuaccacagcgccaucgacgugggcgccagaagagugaaccuggccaucgugagcagacagagagccgacaacguguacgugcugaggccuaccaccguggccagcagaccugugaucggcaucggccugggcaacgacguguuccugaccgcccacgcucuggccuccgguggccccgacgcugccgccaucgugagagugaccaucaacuucuucagacagccucagaugagacaccugagcugguuccuggccggcgacuucaacagaagcccugacagacuggagaacgaccugaugaccgagcaccuggagagagugguggccgugcuggccccuaccgagccuacccagaucggcggcggcauccuggacuacggcgugaucguggacagagccccuuacagccagagaguggaggcccugagaaacccucagcuggccagcgaccacuacccuguggccuuccuggccagaagcugccaucaccaccaccaccac

SEQ ID NO: 103 - protein, 230 aa, Artificial Sequence
  Synthetic Polypeptide
METPAQLLFLLLLWLPDTTGGAYVENREAYNLASDQMEFMLRVGYNSDMGAGIMLTNTYTLQRDDELKHGYNEIEGWYPLFKPTDKLTIQPGGLINDKSIGSGGAVYLDINYKFTPWFNLTVRNRYNHNNYSSTDLNGELDNNDSYEIGNYWNFIITDKFSYTFEPHYFYNVNDFNSSNGTKHHWEITNTFRYRINEHWLPYFELRWLDRNVGLYHREQNQIRIGAKYFF

SEQ ID NO: 104 - RNA, 690 nt, Artificial Sequence
  Synthetic Polynucleotide
auggagacgccugcccagcugcuguuccugcuccuucuguggcugccugacaccaccggcggcgccuacguggagaacagagaggccuacaaccuggccagcgaccagauggaguucaugcugagagugggcuacaacagcgacaugggcgccggcaucaugcuuaccaacaccuacacccugcagagagacgacgagcugaagcacggcuacaacgagaucgagggcugguacccucuguucaagccuaccgacaagcugaccauccagccuggcggccugaucaacgacaagagcaucggcucuggcggugccguguaccuggacaucaacuacaaguucaccccuugguucaaccugaccgugagaaacagauacaaccacaacaacuacagcagcaccgaccugaacggcgagcuggacaacaacgacagcuacgagauuggcaacuacuggaacuucaucaucaccgauaaguucagcuacaccuucgagccucacuacuucuacaacgugaacgacuucaauaguagcaacggcaccaagcaccacugggagaucaccaacacguucagauacagaaucaacgagcacuggcuuccuuacuucgagcugagguggcuggacagaaacgugggccuguaccacagagagcagaaccagaucagaaucggcgccaaguacuucuuc

SEQ ID NO: 105 - protein, 236 aa, Artificial Sequence
  Synthetic Polypeptide
METPAQLLFLLLLWLPDTTGGAYVENREAYNLASDQMEFMLRVGYNSDMGAGIMLTNTYTLQRDDELKHGYNEIEGWYPLFKPTDKLTIQPGGLINDKSIGSGGAVYLDINYKFTPWFNLTVRNRYNHNNYSSTDLNGELDNNDSYEIGNYWNFIITDKFSYTFEPHYFYNVNDFNSSNGTKHHWEITNTFRYRINEHWLPYFELRWLDRNVGLYHREQNQIRIGAKYFFHHHHHH

SEQ ID NO: 106 - RNA, 708 nt, Artificial Sequence
  Synthetic Polynucleotide
auggagacgccugcccagcugcuguuccugcuccuucuguggcugccugacaccaccggcggcgccuacguggagaacagagaggccuacaaccuggccagcgaccagauggaguucaugcugagagugggcuacaacagcgacaugggcgccggcaucaugcuuaccaacaccuacacccugcagagagacgacgagcugaagcacggcuacaacgagaucgagggcugguacccucuguucaagccuaccgacaagcugaccauccagccuggcggccugaucaacgacaagagcaucggcucuggcggugccguguaccuggacaucaacuacaaguucaccccuugguucaaccugaccgugagaaacagauacaaccacaacaacuacagcagcaccgaccugaacggcgagcuggacaacaacgacagcuacgagauuggcaacuacuggaacuucaucaucaccgauaaguucagcuacaccuucgagccucacuacuucuacaacgugaacgacuucaauaguagcaacggcaccaagcaccacugggagaucaccaacacguucagauacagaaucaacgagcacuggcuuccuuacuucgagcugagguggcuggacagaaacgugggccuguaccacagagagcagaaccagaucagaaucggcgccaaguacuucuuccaccaucaccaccaccac

SEQ ID NO: 107 - protein, 230 aa, Artificial Sequence
  Synthetic Polypeptide
METPAQLLFLLLLWLPDTTGGAYVENREAYNLASDQMEFMLRVGYNSDMGAGIMLTNTYTLQRDDELKHGYNEIEGWYPLFKPTDKLTIQPGGLINDKSIGSGGAVYLDINYKFTPWFNLAVRNRYNHNNYASTDLNGELDNNDSYEIGNYWNFIITDKFSYTFEPHYFYNVNDFNSSNGAKHHWEITNTFRYRINEHWLPYFELRWLDRNVGLYHREQNQIRIGAKYFF

SEQ ID NO: 108 - RNA, 690 nt, Artificial Sequence
  Synthetic Polynucleotide
auggagacgccugcccagcugcuguuccugcuccuucuguggcugccugacaccaccggcggcgccuacguggagaacagagaggccuacaaccuggccagcgaccagauggaguucaugcugagagugggcuacaacagcgacaugggcgccggcaucaugcuuaccaacaccuacacccugcagagagacgacgagcugaagcacggcuacaacgagaucgagggcugguacccucuguucaagccuaccgacaagcugaccauccagccuggcggccugaucaacgacaagagcaucggcucuggcggugccguguaccuggacaucaacuacaaguucaccccuugguucaaccuggccgugagaaacagauacaaccacaacaacuacgcaagcaccgaccugaacggcgagcuggacaacaacgacagcuacgagauuggcaacuacuggaacuucaucaucaccgauaaguucagcuacaccuucgagccucacuacuucuacaacgugaacgacuucaauaguagcaacggcgccaagcaccacugggagaucaccaacacguucagauacagaaucaacgagcacuggcuuccuuacuucgagcugagguggcuggacagaaacgugggccuguaccacagagagcagaaccagaucagaaucggcgccaaguacuucuuc

SEQ ID NO: 109 - protein, 236 aa, Artificial Sequence
  Synthetic Polypeptide
METPAQLLFLLLLWLPDTTGGAYVENREAYNLASDQMEFMLRVGYNSDMGAGIMLTNTYTLQRDDELKHGYNEIEGWYPLFKPTDKLTIQPGGLINDKSIGSGGAVYLDINYKFTPWFNLAVRNRYNHNNYASTDLNGELDNNDSYEIGNYWNFIITDKFSYTFEPHYFYNVNDFNSSNGAKHHWEITNTFRYRINEHWLPYFELRWLDRNVGLYHREQNQIRIGAKYFFHHHHHH

SEQ ID NO: 110 - RNA, 708 nt, Artificial Sequence
  Synthetic Polynucleotide
auggagacgccugcccagcugcuguuccugcuccuucuguggcugccugacaccaccggcggcgccuacguggagaacagagaggccuacaaccuggccagcgaccagauggaguucaugcugagagugggcuacaacagcgacaugggcgccggcaucaugcuuaccaacaccuacacccugcagagagacgacgagcugaagcacggcuacaacgagaucgagggcugguacccucuguucaagccuaccgacaagcugaccauccagccuggcggccugaucaacgacaagagcaucggcucuggcggugccguguaccuggacaucaacuacaaguucaccccuugguucaaccuggccgugagaaacagauacaaccacaacaacuacgcaagcaccgaccugaacggcgagcuggacaacaacgacagcuacgagauuggcaacuacuggaacuucaucaucaccgauaaguucagcuacaccuucgagccucacuacuucuacaacgugaacgacuucaauaguagcaacggcgccaagcaccacugggagaucaccaacacguucagauacagaaucaacgagcacuggcuuccuuacuucgagcugagguggcuggacagaaacgugggccuguaccacagagagcagaaccagaucagaaucggcgccaaguacuucuuccaccaucaccaccaccac

SEQ ID NO: 111 - protein, 244 aa, Artificial Sequence
  Synthetic Polypeptide
METPAQLLFLLLLWLPDTTGVDFVYRVDSTPPDVIFRDGFSLLGYNRNFQQFISGRSCSGGSSDSRYIATTSSVNQTYAIARAYYSRSTFKGNLYRYQIRADNNFYSLLPSITYLETQGGHFNAYEKTMMRLQREYVSTLSILPENIQKAVALVYDSATGLVKDGVSTMNASYLGLSTTSNPGVIPFLPEPQTYTQQRIDAFGPLISSCFSIGSVCHSHRGQRADVYNMSFYDARPVIELILSK

SEQ ID NO: 112 - RNA, 732 nt, Artificial Sequence
  Synthetic Polynucleotide
auggaaaccccugcccagcugcuguuccugcugcugcucuggcugccugacacaaccggcguggacuucguguacagaguggacagcaccccuccugacgugaucuucagagacggcuucagccugcugggcuacaacagaaacuuccagcaguucaucagcggcagaagcugcagcggcgguagcagcgauagcagauacaucgccaccaccagcagcgugaaccagaccuacgccaucgccagagccuacuacagcagaagcaccuucaagggcaaccuguacagauaccagaucagagccgacaacaacuucuacagccugcugccuagcaucaccuaccuggaaacgcagggcggccacuucaacgccuacgagaagaccaugaugagacugcagagagaguacgugagcacccugucaauccugcccgagaacauccagaaggccguggcccugguguacgacagcgccaccggccuggugaaggacggcgugagcaccaugaacgccagcuaccucgggcugagcacaaccagcaacccuggcgugaucccuuuccugccugagccucagaccuacacccagcagagaaucgacgccuucggcccucugaucagcagcugcuucagcaucggcagcgugugccacagccacagaggccagagagccgacguguacaacaugagcuucuacgacgccagaccugugaucgagcugauccuguccaag

SEQ ID NO: 113 - protein, 250 aa, Artificial Sequence
  Synthetic Polypeptide
METPAQLLFLLLLWLPDTTGVDFVYRVDSTPPDVIFRDGFSLLGYNRNFQQFISGRSCSGGSSDSRYIATTSSVNQTYAIARAYYSRSTFKGNLYRYQIRADNNFYSLLPSITYLETQGGHFNAYEKTMMRLQREYVSTLSILPENIQKAVALVYDSATGLVKDGVSTMNASYLGLSTTSNPGVIPFLPEPQTYTQQRIDAFGPLISSCFSIGSVCHSHRGQRADVYNMSFYDARPVIELILSKHHHHHH

SEQ ID NO: 114 - RNA, 750 nt, Artificial Sequence
  Synthetic Polynucleotide
auggaaaccccugcccagcugcuguuccugcugcugcucuggcugccugacacaaccggcguggacuucguguacagaguggacagcaccccuccugacgugaucuucagagacggcuucagccugcugggcuacaacagaaacuuccagcaguucaucagcggcagaagcugcagcggcgguagcagcgauagcagauacaucgccaccaccagcagcgugaaccagaccuacgccaucgccagagccuacuacagcagaagcaccuucaagggcaaccuguacagauaccagaucagagccgacaacaacuucuacagccugcugccuagcaucaccuaccuggaaacgcagggcggccacuucaacgccuacgagaagaccaugaugagacugcagagagaguacgugagcacccugucaauccugcccgagaacauccagaaggccguggcccugguguacgacagcgccaccggccuggugaaggacggcgugagcaccaugaacgccagcuaccucgggcugagcacaaccagcaacccuggcgugaucccuuuccugccugagccucagaccuacacccagcagagaaucgacgccuucggcccucugaucagcagcugcuucagcaucggcagcgugugccacagccacagaggccagagagccgacguguacaacaugagcuucuacgacgccagaccugugaucgagcugauccuguccaagcaccaccaccaucaccac

SEQ ID NO: 115 - protein, 244 aa, Artificial Sequence
  Synthetic Polypeptide
METPAQLLFLLLLWLPDTTGVDFVYRVDSTPPDVIFRDGFSLLGYNRNFQQFISGRSCSGGSSDSRYIATTSSVNQAYAIARAYYSRSTFKGNLYRYQIRADNNFYSLLPSITYLETQGGHFNAYEKTMMRLQREYVSTLSILPENIQKAVALVYDSATGLVKDGVSTMNASYLGLSTTSNPGVIPFLPEPQTYTQQRIDAFGPLISSCFSIGSVCHSHRGQRADVYNMSFYDARPVIELILSK

SEQ ID NO: 116 - RNA, 732 nt, Artificial Sequence
  Synthetic Polynucleotide
auggaaaccccugcccagcugcuguuccugcugcugcucuggcugccugacacaaccggcguggacuucguguacagaguggacagcaccccuccugacgugaucuucagagacggcuucagccugcugggcuacaacagaaacuuccagcaguucaucagcggcagaagcugcagcggcgguagcagcgauagcagauacaucgccaccaccagcagcgugaaccaggccuacgccaucgccagagccuacuacagcagaagcaccuucaagggcaaccuguacagauaccagaucagagccgacaacaacuucuacagccugcugccuagcaucaccuaccuggaaacgcagggcggccacuucaacgccuacgagaagaccaugaugagacugcagagagaguacgugagcacccugucaauccugcccgagaacauccagaaggccguggcccugguguacgacagcgccaccggccuggugaaggacggcgugagcaccaugaacgccagcuaccucgggcugagcacaaccagcaacccuggcgugaucccuuuccugccugagccucagaccuacacccagcagagaaucgacgccuucggcccucugaucagcagcugcuucagcaucggcagcgugugccacagccacagaggccagagagccgacguguacaacaugagcuucuacgacgccagaccugugaucgagcugauccuguccaag

SEQ ID NO: 117 - protein, 250 aa, Artificial Sequence
  Synthetic Polypeptide
METPAQLLFLLLLWLPDTTGVDFVYRVDSTPPDVIFRDGFSLLGYNRNFQQFISGRSCSGGSSDSRYIATTSSVNQAYAIARAYYSRSTFKGNLYRYQIRADNNFYSLLPSITYLETQGGHFNAYEKTMMRLQREYVSTLSILPENIQKAVALVYDSATGLVKDGVSTMNASYLGLSTTSNPGVIPFLPEPQTYTQQRIDAFGPLISSCFSIGSVCHSHRGQRADVYNMSFYDARPVIELILSKHHHHHH

SEQ ID NO: 118 - RNA, 750 nt, Artificial Sequence
  Synthetic Polynucleotide
auggaaaccccugcccagcugcuguuccugcugcugcucuggcugccugacacaaccggcguggacuucguguacagaguggacagcaccccuccugacgugaucuucagagacggcuucagccugcugggcuacaacagaaacuuccagcaguucaucagcggcagaagcugcagcggcgguagcagcgauagcagauacaucgccaccaccagcagcgugaaccaggccuacgccaucgccagagccuacuacagcagaagcaccuucaagggcaaccuguacagauaccagaucagagccgacaacaacuucuacagccugcugccuagcaucaccuaccuggaaacgcagggcggccacuucaacgccuacgagaagaccaugaugagacugcagagagaguacgugagcacccugucaauccugcccgagaacauccagaaggccguggcccugguguacgacagcgccaccggccuggugaaggacggcgugagcaccaugaacgccagcuaccucgggcugagcacaaccagcaacccuggcgugaucccuuuccugccugagccucagaccuacacccagcagagaaucgacgccuucggcccucugaucagcagcugcuucagcaucggcagcgugugccacagccacagaggccagagagccgacguguacaacaugagcuucuacgacgccagaccugugaucgagcugauccuguccaagcaccaccaccaucaccac

SEQ ID NO: 119 - protein, 134 aa, Artificial Sequence
  Synthetic Polypeptide
METPAQLLFLLLLWLPDTTGEWTGDNTNAYYSDEVISELHVGQIDTSPYFCIKTVKANGSGTPVVACAVSKQSIWAPSFKELLDQARYFYSTGQSVRIHVQKNIWTYPLFVNTFSANALVGLSSCSATQCFGPK

SEQ ID NO: 120 - RNA, 402 nt, Artificial Sequence
  Synthetic Polynucleotide
auggagacaccugcccagcugcuguuccugcugcugcuguggcugccugacaccacuggcgaguggaccggcgacaacaccaacgccuacuacagcgacgaggugaucuccgagcugcacgugggacagaucgacaccagcccuuacuucugcaucaagaccgugaaggccaacggcagcggcaccccugugguggccugcgccgugagcaagcagagcaucugggccccuagcuucaaggagcugcuggaccaggccagauacuucuacagcaccggccagagcgugagaauccacgugcagaagaacaucuggaccuacccucuguucgugaacaccuucagcgcgaacgcccuggugggccugagcagcugcagcgccacccagugcuucggccccaag

SEQ ID NO: 121 - protein, 140 aa, Artificial Sequence
  Synthetic Polypeptide
METPAQLLFLLLLWLPDTTGEWTGDNTNAYYSDEVISELHVGQIDTSPYFCIKTVKANGSGTPVVACAVSKQSIWAPSFKELLDQARYFYSTGQSVRIHVQKNIWTYPLFVNTFSANALVGLSSCSATQCFGPKHHHHHH

SEQ ID NO: 122 - RNA, 420 nt, Artificial Sequence
  Synthetic Polynucleotide
auggagacaccugcccagcugcuguuccugcugcugcuguggcugccugacaccacuggcgaguggaccggcgacaacaccaacgccuacuacagcgacgaggugaucuccgagcugcacgugggacagaucgacaccagcccuuacuucugcaucaagaccgugaaggccaacggcagcggcaccccugugguggccugcgccgugagcaagcagagcaucugggccccuagcuucaaggagcugcuggaccaggccagauacuucuacagcaccggccagagcgugagaauccacgugcagaagaacaucuggaccuacccucuguucgugaacaccuucagcgcgaacgcccuggugggccugagcagcugcagcgccacccagugcuucggccccaagcaccaucaccaccaccac

SEQ ID NO: 123 - protein, 134 aa, Artificial Sequence
  Synthetic Polypeptide
METPAQLLFLLLLWLPDTTGEWTGDNTNAYYSDEVISELHVGQIDTSPYFCIKTVKANGAGTPVVACAVSKQSIWAPSFKELLDQARYFYSTGQSVRIHVQKNIWTYPLFVNTFSANALVGLSSCSATQCFGPK

SEQ ID NO: 124 - RNA, 402 nt, Artificial Sequence
  Synthetic Polynucleotide
auggagacaccugcccagcugcuguuccugcugcugcuguggcugccugacaccacuggcgaguggaccggcgacaacaccaacgccuacuacagcgacgaggugaucuccgagcugcacgugggacagaucgacaccagcccuuacuucugcaucaagaccgugaaggccaacggcgcaggcaccccugugguggccugcgccgugagcaagcagagcaucugggccccuagcuucaaggagcugcuggaccaggccagauacuucuacagcaccggccagagcgugagaauccacgugcagaagaacaucuggaccuacccucuguucgugaacaccuucagcgcgaacgcccuggugggccugagcagcugcagcgccacccagugcuucggccccaag

SEQ ID NO: 125 - protein, 140 aa, Artificial Sequence
  Synthetic Polypeptide
METPAQLLFLLLLWLPDTTGEWTGDNTNAYYSDEVISELHVGQIDTSPYFCIKTVKANGAGTPVVACAVSKQSIWAPSFKELLDQARYFYSTGQSVRIHVQKNIWTYPLFVNTFSANALVGLSSCSATQCFGPKHHHHHH

SEQ ID NO: 126 - RNA, 420 nt, Artificial Sequence
  Synthetic Polynucleotide
auggagacaccugcccagcugcuguuccugcugcugcuguggcugccugacaccacuggcgaguggaccggcgacaacaccaacgccuacuacagcgacgaggugaucuccgagcugcacgugggacagaucgacaccagcccuuacuucugcaucaagaccgugaaggccaacggcgcaggcaccccugugguggccugcgccgugagcaagcagagcaucugggccccuagcuucaaggagcugcuggaccaggccagauacuucuacagcaccggccagagcgugagaauccacgugcagaagaacaucuggaccuacccucuguucgugaacaccuucagcgcgaacgcccuggugggccugagcagcugcagcgccacccagugcuucggccccaagcaccaucaccaccaccac

SEQ ID NO: 127 - protein, 434 aa, Artificial Sequence
  Synthetic Polypeptide
METPAQLLFLLLLWLPDTTGMNTQALFCLSLPLVIAGCAQQTSTQTARDSAFAQSQQPSVFSDIYQRSPEVTRSGRYTLVSIKSADAQREPLNQLIDITMPGQLVNSVGDGFRYLLFQSGYSLCGHYGADFSELLNRPLPAVQRKIGPMRLSEALQVVAGPAWRMSVDEVNREVCFVLRDAYRVQAKTKALTSVTPAVTGAARTSSPETTRNPYTGVLPGNKAEPVIAGQYLAVKKGTELKPVSQSLPVPALRPGGTPVTSGVSSAPPVKQVSSPAPRNPFTGKNLTGTTLSASHSPAPVGEKAQAKTPSPSTVKAVNPATATVLATTTKPLTGTPVAAVASGPEWKAVVGSTLKESLTDWAGRADCPGGGHWVVIWQTPTDYRIDAPLVFKGNFETALVQVFDLYKKADKPLFAEASRLQCLVSVADKPADRS

SEQ ID NO: 128 - RNA, 1302 nt, Artificial Sequence
  Synthetic Polynucleotide
auggaaaccccugcccagcugcuguuccugcugcugcuguggcugccugacaccaccggcaugaacacccaggcccuguucugccugagccugccucucgucaucgccgguugcgcccagcagaccagcacccagaccgccagagacagcgccuucgcccagagccagcagccuagcguguucagcgacaucuaccagagaagcccugaggugaccagaagcggcagauacacccuggugagcaucaagagcgccgacgcccagagagagccucugaaccagcugaucgacaucaccaugccuggccagcuggugaacagcgugggcgacggcuucagauaccugcuguuccagagcggcuacagccugugcggccacuacggagccgacuucagcgagcugcugaacagaccucugccugccgugcagagaaagaucggcccuaugagacugagcgaagcccugcagguggucgcgggcccugccuggagaaugagcguggacgaggugaacagagaggugugcuucgugcugagagacgccuacagagugcaggccaagaccaaggcccugaccagcgugacuccagccguuaccggcgccgcccggacauccuccccugagacuaccagaaacccuuacaccggcgugcugccuggcaacaaggccgagcccgugaucgccggccaguaccuggccgugaagaagggcaccgagcugaagccugugagccagagccugccugugccugcccugaggccuggcggcaccccugugaccucgggcguguccucugccccuccugugaagcaggugagcagccccgccccuagaaacccuuucaccggcaagaaccugaccggcaccacccugagcgccagccacagucccgcuccggugggcgagaaggcccaggcaaagacgccuagcccuagcaccgugaaggccgugaacccugccaccgccaccgugcucgccaccaccacuaagccucugaccggaacucccguggccgccguagccagcggaccugaguggaaagcugucgugggcucgacccugaaggagagccugaccgacugggccggcagagccgauugccccggcggcgggcacuggguggugaucuggcagaccccuaccgacuacagaaucgacgccccucugguguucaagggcaacuucgagacagcccuggugcagguguucgaccuguacaagaaggccgacaagccccuguucgccgaggccagcagacugcagugccugguguccguggccgacaaacccgccgacagaagc

SEQ ID NO: 129 - RNA, 119 nt, Artificial Sequence
  Synthetic Polynucleotide
ugauaauaggcuggagccucgguggccuagcuucuugccccuugggccuccccccagccccuccuccccuuccugcacccguacccccguggucuuugaauaaagucugagugggcggc

SEQ ID NO: 130 - protein, 434 aa, Artificial Sequence
  Synthetic Polypeptide
METPAQLLFLLLLWLPDTTGMNTQALFCLSLPLVIAGCAQQTSTQTARDSAFAQSQQPSVFSDIYQRSPEVTRSGRYTLVSIKSADAQREPLNQLIDITMPGQLVNSVGDGFRYLLFQSGYSLCGHYGADFSELLNRPLPAVQRKIGPMRLSEALQVVAGPAWRMSVDEVNREVCFVLRDAYRVQAKTKALTSVTPAVTGAARTSSPETTRNPYTGVLPGNKAEPVIAGQYLAVKKGTELKPVSQSLPVPALRPGGTPVTSGVSSAPPVKQVSSPAPRNPFTGKNLAGTTLSASHSPAPVGEKAQAKTPSPSTVKAVNPATATVLATTTKPLTGTPVAAVASGPEWKAVVGSTLKESLTDWAGRADCPGGGHWVVIWQTPTDYRIDAPLVFKGNFETALVQVFDLYKKADKPLFAEASRLQCLVSVADKPADRS

SEQ ID NO: 131 - RNA, 1302 nt, Artificial Sequence
  Synthetic Polynucleotide
auggaaaccccugcccagcugcuguuccugcugcugcuguggcugccugacaccaccggcaugaacacccaggcccuguucugccugagccugccucucgucaucgccgguugcgcccagcagaccagcacccagaccgccagagacagcgccuucgcccagagccagcagccuagcguguucagcgacaucuaccagagaagcccugaggugaccagaagcggcagauacacccuggugagcaucaagagcgccgacgcccagagagagccucugaaccagcugaucgacaucaccaugccuggccagcuggugaacagcgugggcgacggcuucagauaccugcuguuccagagcggcuacagccugugcggccacuacggagccgacuucagcgagcugcugaacagaccucugccugccgugcagagaaagaucggcccuaugagacugagcgaagcccugcagguggucgcgggcccugccuggagaaugagcguggacgaggugaacagagaggugugcuucgugcugagagacgccuacagagugcaggccaagaccaaggcccugaccagcgugacuccagccguuaccggcgccgcccggacauccuccccugagacuaccagaaacccuuacaccggcgugcugccuggcaacaaggccgagcccgugaucgccggccaguaccuggccgugaagaagggcaccgagcugaagccugugagccagagccugccugugccugcccugaggccuggcggcaccccugugaccucgggcguguccucugccccuccugugaagcaggugagcagccccgccccuagaaacccuuucaccggcaagaaccuggccggcaccacccugagcgccagccacagucccgcuccggugggcgagaaggcccaggcaaagacgccuagcccuagcaccgugaaggccgugaacccugccaccgccaccgugcucgccaccaccacuaagccucugaccggaacucccguggccgccguagccagcggaccugaguggaaagcugucgugggcucgacccugaaggagagccugaccgacugggccggcagagccgauugccccggcggcgggcacuggguggugaucuggcagaccccuaccgacuacagaaucgacgccccucugguguucaagggcaacuucgagacagcccuggugcagguguucgaccuguacaagaaggccgacaagccccuguucgccgaggccagcagacugcagugccugguguccguggccgacaaacccgccgacagaagc

SEQ ID NO: 132 - protein, 484 aa, Artificial Sequence
  Synthetic Polypeptide
METPAQLLFLLLLWLPDTTGQTTNRSTAKDEILTGSYIDPTKTRFPLADYSQSVDKWIPPDSADYTIPVIDSATQQRYFSALKSHYFGMDSEAHSPWNDFYITALLKKNAAQARDASIKQFLSDGSAYWGENFRLYTSRWKEEVRGNTDTQIDNIYHASRRGIMVRESLVRALPTDDPLFNDPRQAGEGYPFDNLQMSSLRPGTPVYTLTKSKDQRWQYVVSPAVTGWVHSEDIASTDQKFITQWVLLAHKQLGAFINAPVSVHAAGVYYFTGRPGTILPFRHQRAGQFLIAAPVRGSNGRAFIHWVWLSGNEFTAMPWKMTPENIAVLMKAMHGAPYGWGNFNFYNDCSAEVRSLLMPFGIFLPRHSSAQVESAGRVVDLSHKNPQMRIDYLTRYGKAFTTLVYIPGHIMLYIGNTAMNGQVVPMTYQNIWGLRPNHANSRSIIGEAVFLPLLRFYPENPELISLAGKVLFKLGYIEHHHHHH

SEQ ID NO: 133 - RNA, 1452 nt, Artificial Sequence
  Synthetic Polynucleotide
auggagacuccugcccagcugcuguuccugcugcugcuguggcugccugacaccaccggccagaccaccaacagaagcaccgccaaggacgagauccugaccggcagcuacaucgacccuaccaagaccagauuuccccuggcugauuauagccagagcguggacaaguggaucccuccugacucagccgacuacaccaucccugugaucgacagcgccacccagcagagauacuucagcgcccugaagucccacuacuucggcauggacagcgaggcccacagcccuuggaacgacuucuacaucaccgcccugcugaagaagaacgccgcccaggccagagacgccagcaucaagcaguuucuguccgacggcagcgccuacuggggcgagaacuucagacuguacaccagccgguggaaggaggaggugagaggcaacaccgacacccagaucgacaacaucuaccacgccagccgaaggggcaucauggugagagagagccuggugagagcccugccuaccgacgacccucuguucaacgacccuagacaggccggcgagggcuacccuuucgacaaccugcagaugagcagccugcgcccuggcaccccuguguacacccugaccaagagcaaggaccagcgguggcaguacgugguguccccugccgugacagggugggugcacagcgaggacaucgccagcaccgaccagaaguucaucacccagugggugcugcuggcccauaagcagcucggcgccuucaucaacgccccuguuuccgugcacgccgccggcguguacuacuucaccggcagacccggaaccauccugccuuucagacaccagagagccgggcaguuccugauagccgccccgguacgcgggagcaacggccgggccuuuauccacuggguguggcugagcggcaacgaguucaccgccaugccuuggaagaugaccccugagaacaucgccguccugaugaaggccaugcacggcgccccuuacggcuggggcaacuucaacuucuacaacgacugcagcgccgaggugagaagccugcugaugccuuucggcaucuuccugccccgucacuccagcgcccaggucgagagcgccggcagagugguggaccugagccacaagaacccucagaugagaaucgacuaccugaccagauacggcaaggccuucaccacacucguguacauccccggccacaucaugcuguacaucggcaacacagccaugaacggccagguggugccuaugaccuaccagaacaucuggggccucagaccuaaccacgccaacagcagaagcaucaucggcgaggccguguuccugccucugcugagauucuacccugagaacccugagcugaucagccuggccggcaaggugcuguucaagcugggcuacaucgagcaccaucaccaccaccac

SEQ ID NO: 134 - protein, 451 aa, Artificial Sequence
  Synthetic Polypeptide
METPAQLLFLLLLWLPDTTGVDKWIPPDSADYTIPVIDSATQQRYFSALKSHYFGMDSEAHSPWNDFYITALLKKNAAQARDASIKQFLSDGSAYWGENFRLYTSRWKEEVRGNTDTQIDNIYHASRRGIMVRESLVRALPTDDPLFNDPRQAGEGYPFDNLQMSSLRPGTPVYTLTKSKDQRWQYVVSPAVTGWVHSEDIASTDQKFITQWVLLAHKQLGAFINAPVSVHAAGVYYFTGRPGTILPFRHQRAGQFLIAAPVRGSNGRAFIHWVWLSGNEFTAMPWKMTPENIAVLMKAMHGAPYGWGNFNFYNDCSAEVRSLLMPFGIFLPRHSSAQVESAGRVVDLSHKNPQMRIDYLTRYGKAFTTLVYIPGHIMLYIGNTAMNGQVVPMTYQNIWGLRPNHANSRSIIGEAVFLPLLRFYPENPELISLAGKVLFKLGYIEHHHHHH

SEQ ID NO: 135 - RNA, 1353 nt, Artificial Sequence
  Synthetic Polynucleotide
auggagacuccugcccagcugcuguuccugcugcugcuguggcugccugacaccaccggcguggacaaguggaucccuccugacucagccgacuacaccaucccugugaucgacagcgccacccagcagagauacuucagcgcccugaagucccacuacuucggcauggacagcgaggcccacagcccuuggaacgacuucuacaucaccgcccugcugaagaagaacgccgcccaggccagagacgccagcaucaagcaguuucuguccgacggcagcgccuacuggggcgagaacuucagacuguacaccagccgguggaaggaggaggugagaggcaacaccgacacccagaucgacaacaucuaccacgccagccgaaggggcaucauggugagagagagccuggugagagcccugccuaccgacgacccucuguucaacgacccuagacaggccggcgagggcuacccuuucgacaaccugcagaugagcagccugcgcccuggcaccccuguguacacccugaccaagagcaaggaccagcgguggcaguacgugguguccccugccgugacagggugggugcacagcgaggacaucgccagcaccgaccagaaguucaucacccagugggugcugcuggcccauaagcagcucggcgccuucaucaacgccccuguuuccgugcacgccgccggcguguacuacuucaccggcagacccggaaccauccugccuuucagacaccagagagccgggcaguuccugauagccgccccgguacgcgggagcaacggccgggccuuuauccacuggguguggcugagcggcaacgaguucaccgccaugccuuggaagaugaccccugagaacaucgccguccugaugaaggccaugcacggcgccccuuacggcuggggcaacuucaacuucuacaacgacugcagcgccgaggugagaagccugcugaugccuuucggcaucuuccugccccgucacuccagcgcccaggucgagagcgccggcagagugguggaccugagccacaagaacccucagaugagaaucgacuaccugaccagauacggcaaggccuucaccacacucguguacauccccggccacaucaugcuguacaucggcaacacagccaugaacggccagguggugccuaugaccuaccagaacaucuggggccucagaccuaaccacgccaacagcagaagcaucaucggcgaggccguguuccugccucugcugagauucuacccugagaacccugagcugaucagccuggccggcaaggugcuguucaagcugggcuacaucgagcaccaucaccaccaccac

SEQ ID NO: 136 - protein, 160 aa, Artificial Sequence
  Synthetic Polypeptide
METPAQLLFLLLLWLPDTTGDSLSGDVYTASEAKQVQNVAYGTIVNVRPVQIQGGDDSNVIGAIGGAVLGGFLGNTIGGGTGRSLATAAGAVAGGVAGQGVQSAMNKAQGVELEIRKDDGNTIMVVQKQGNTRFSAGQRVVLASNGAQVTVSPRHHHHHH

SEQ ID NO: 137 - RNA, 480 nt, Artificial Sequence
  Synthetic Polynucleotide
auggaaacgccugcccagcugcuguuccugcugcugcuguggcugccugacaccaccggcgacagccugagcggcgacguguacaccgccagcgaggccaagcaggugcagaacguggccuacggcaccaucgugaacgucagaccugugcagauccagggcggggacgacagcaacgugaucggcgccauuggaggugccgugcugggcggcuuccugggcaacacuaucggcggcggcaccggcagaagccuggccacagccgccggagccguggcuggcggcguggccggucagggagugcagagcgccaugaacaaggcccagggcguggagcuggagaucagaaaggacgacggcaacaccaucaugguggugcagaagcaagggaacaccagauucagcgccggccagagaguggugcuggccagcaacggcgcccaggugaccgugagcccucgccaucaccaccaccaccac

SEQ ID NO: 138 - protein, 265 aa, Artificial Sequence
  Synthetic Polypeptide
METPAQLLFLLLLWLPDTTGYKVMTWNLQGSSASTESKWNVNVRQLLSGTAGVDILMVQEAGAVPTSAVPTGRHIQPFGVGIPIDEYTWNLGTTSRQDIRYIYHSAIDVGARRVNLAIVSRQRADNVYVLRPTTVASRPVIGIGLGNDVFLTAHALASGGPDAAAIVRVTINFFRQPQMRHLSWFLAGDFNRSPDRLENDLMTEHLERVVAVLAPTEPTQIGGGILDYGVIVDRAPYSQRVEALRNPQLASDHYPVAFLHHHHHH

SEQ ID NO: 139 - RNA, 795 nt, Artificial Sequence
  Synthetic Polynucleotide
auggagacuccugcccagcugcuguuccugcugcugcuguggcugccugacaccaccggcuacaaggugaugaccuggaaccugcagggaagcuccgccagcaccgagagcaaguggaacgugaacgugagacagcuccugagcggcaccgccggcgucgacauccugauggugcaggaggccggagccgucccuaccagcgccgugccuaccggcagacacauccagccuuucggcgugggcaucccuaucgacgaguacaccuggaaucucggcaccaccagcagacaggacaucagauacaucuaccacagcgccaucgacgugggcgccagaagagugaaccuggccaucgugagcagacagagagccgacaacguguacgugcugaggccuaccaccguggccagcagaccugugaucggcaucggccugggcaacgacguguuccugaccgcccacgcucuggccuccgguggccccgacgcugccgccaucgugagagugaccaucaacuucuucagacagccucagaugagacaccugagcugguuccuggccggcgacuucaacagaagcccugacagacuggagaacgaccugaugaccgagcaccuggagagagugguggccgugcuggccccuaccgagccuacccagaucggcggcggcauccuggacuacggcgugaucguggacagagccccuuacagccagagaguggaggcccugagaaacccucagcuggccagcgaccacuacccuguggccuuccugcaucaccaccaccaccac

SEQ ID NO: 140 - RNA, 57 nt, Artificial Sequence
  Synthetic Polynucleotide
gggaaauaagagagaaaagaagaguaagaagaaauauaagaccccggcgccgccacc

SEQ ID NO: 141 - protein, 265 aa, Artificial Sequence
  Synthetic Polypeptide
METPAQLLFLLLLWLPDTTGYKVMTWNLQGSSASTESKWNVNVRQLLSGTAGVDILMVQEAGAVPTSAVPTGRHIQPFGVGIPIDEYTWNLGTTSRQDIRYIYHSAIDVGARRVNLAIVSRQRADNVYVLRPTTVASRPVIGIGLGNDVFLTAQALASGGPDAAAIVRVTINFFRQPQMRHLSWFLAGDFNRSPDRLENDLMTEHLERVVAVLAPTEPTQIGGGILDYGVIVDRAPYSQRVEALRNPQLASDHYPVAFLHHHHHH

SEQ ID NO: 142 - RNA, 795 nt, Artificial Sequence
  Synthetic Polynucleotide
auggagacuccugcccagcugcuguuccugcugcugcuguggcugccugacaccaccggcuacaaggugaugaccuggaaccugcagggaagcuccgccagcaccgagagcaaguggaacgugaacgugagacagcuccugagcggcaccgccggcgucgacauccugauggugcaggaggccggagccgucccuaccagcgccgugccuaccggcagacacauccagccuuucggcgugggcaucccuaucgacgaguacaccuggaaucucggcaccaccagcagacaggacaucagauacaucuaccacagcgccaucgacgugggcgccagaagagugaaccuggccaucgugagcagacagagagccgacaacguguacgugcugaggccuaccaccguggccagcagaccugugaucggcaucggccugggcaacgacguguuccugaccgcccaggcucuggccuccgguggccccgacgcugccgccaucgugagagugaccaucaacuucuucagacagccucagaugagacaccugagcugguuccuggccggcgacuucaacagaagcccugacagacuggagaacgaccugaugaccgagcaccuggagagagugguggccgugcuggccccuaccgagccuacccagaucggcggcggcauccuggacuacggcgugaucguggacagagccccuuacagccagagaguggaggcccugagaaacccucagcuggccagcgaccacuacccuguggccuuccugcaucaccaccaccaccac

SEQ ID NO: 143 - protein, 262 aa, Artificial Sequence
  Synthetic Polypeptide
METPAQLLFLLLLWLPDTTGQAPISSVGSGSVEDRVTQLERISNAHSQLLTQLQQQLSDNQSDIDSLRGQIQENQYQLNQVMERQKQIMLQLGSLNNGGAAQPAAGDQSGAATAATPAPDAGTATSGAPVQSGDANTDYNAAIALVQDKSRQDDAIVAFQNFIKKYPDSTYQPNANYWLGQLNYNKGKKDDAAYYFASVVKNYPKSPKAADAMYKVGVIMQDKGDTAKAKAVYQQVINKYPGTDGAKQAQKRLNAMHHHHHH

SEQ ID NO: 144 - RNA, 786 nt, Artificial Sequence
  Synthetic Polynucleotide
auggagacaccugcccagcugcuguuccugcugcugcuguggcugcccgacaccaccggccaggccccuaucagcagcgugggcagcggaagcguggaggacagagugacccagcuggagagaaucagcaacgcccacagccagcugcucacccagcuccagcagcagcugagcgacaaccagagcgacaucgacagccugagaggccagauccaggagaaccaguaccagcugaaccaggugauggagagacagaagcagaucaugcugcagcugggaagccugaacaacggcggagccgcucagccugcagccggcgaccagaguggcgccgcaacagccgccacaccagccccugacgcggguacagccaccagcggugcucccgugcagagcggcgacgccaacaccgacuacaacgccgccaucgcccuggugcaggacaagagcagacaggacgacgcuaucguggccuuccagaacuucaucaagaaguaccccgacagcaccuaccagcccaacgccaacuacuggcugggccagcugaacuacaacaagggcaagaaggacgacgccgccuacuacuucgccagcguggugaagaacuaccccaagagccccaaagcggccgacgccauguacaaggugggcgugaucaugcaggacaagggcgacaccgccaaggccaaggccguguaccagcaggugaucaacaaguaccccgguacugacggcgccaagcaggcccagaagagacugaacgccaugcaccaucaccaucaccac

SEQ ID NO: 145 - protein, 262 aa, Artificial Sequence
  Synthetic Polynucleotide
METPAQLLFLLLLWLPDTTGQAPISSVGSGSVEDRVTQLERISNAHSQLLTQLQQQLSDNQADIDSLRGQIQENQYQLNQVMERQKQIMLQLGSLNNGGAAQPAAGDQSGAATAATPAPDAGTATSGAPVQSGDANTDYNAAIALVQDKSRQDDAIVAFQNFIKKYPDSTYQPNANYWLGQLNYNKGKKDDAAYYFASVVKNYPKSPKAADAMYKVGVIMQDKGDTAKAKAVYQQVINKYPGTDGAKQAQKRLNAMHHHHHH

SEQ ID NO: 146 - RNA, 786 nt, Artificial Sequence
  Synthetic Polynucleotide
auggagacaccugcccagcugcuguuccugcugcugcuguggcugcccgacaccaccggccaggccccuaucagcagcgugggcagcggaagcguggaggacagagugacccagcuggagagaaucagcaacgcccacagccagcugcucacccagcuccagcagcagcugagcgacaaccaggccgacaucgacagccugagaggccagauccaggagaaccaguaccagcugaaccaggugauggagagacagaagcagaucaugcugcagcugggaagccugaacaacggcggagccgcucagccugcagccggcgaccagaguggcgccgcaacagccgccacaccagccccugacgcggguacagccaccagcggugcucccgugcagagcggcgacgccaacaccgacuacaacgccgccaucgcccuggugcaggacaagagcagacaggacgacgcuaucguggccuuccagaacuucaucaagaaguaccccgacagcaccuaccagcccaacgccaacuacuggcugggccagcugaacuacaacaagggcaagaaggacgacgccgccuacuacuucgccagcguggugaagaacuaccccaagagccccaaagcggccgacgccauguacaaggugggcgugaucaugcaggacaagggcgacaccgccaaggccaaggccguguaccagcaggugaucaacaaguaccccgguacugacggcgccaagcaggcccagaagagacugaacgccaugcaccaucaccaucaccac

SEQ ID NO: 147 - protein, 193 aa, Artificial Sequence
  Synthetic Polypeptide
METPAQLLFLLLLWLPDTTGENKSYYQLPIAQSGVQSTASQGNRLLWVEQVSVPDYLAGNGVVYQTSDVQYVIANNNLWASPLDQQLRNTLVANLSARLPGWVVASQPLGTTQDTLNVTVTGFHGRYDGKVIVSGEWLLNHNGQLIKRPFHIEASQQKDGYDEMVKVLASAWSQEAAAIADEIKRLPHHHHHH

SEQ ID NO: 148 - RNA, 579 nt, Artificial Sequence
  Synthetic Polynucleotide
auggaaaccccagcccagcuccuguuccugcugcugcuguggcuucccgacaccaccggcgagaacaagagcuacuaccagcugcccaucgcccagagcggcgugcagagcacagccagccagggcaacagacugcuguggguggagcaggugagcgugcccgacuaccuggccggcaacggcgugguguaccagaccagcgacgugcaguacgugaucgccaacaacaaccugugggccagcccgcuggaccagcagcugagaaacacccugguggccaaccugagcgccagacuccccggcugggucguugccucccagccccugggcaccacccaggacacccugaacgugaccgugaccggcuuccacggcagauacgacggcaaggugaucgugagcggcgaguggcugcugaaccacaacggccagcugaucaagaggcccuuccacaucgaggccagccagcagaaggacggcuacgacgagauggugaaggugcuggccagcgccuggagccaggaggccgcugccauagccgacgagaucaagagacugccccaccaucaccaccaccac

SEQ ID NO: 149 - protein, 193 aa, Artificial Sequence
  Synthetic Polypeptide
METPAQLLFLLLLWLPDTTGENKAYYQLPIAQSGVQSTASQGNRLLWVEQVSVPDYLAGNGVVYQTSDVQYVIANNNLWASPLDQQLRNTLVANLAARLPGWVVASQPLGTTQDTLNVAVTGFHGRYDGKVIVSGEWLLNHNGQLIKRPFHIEASQQKDGYDEMVKVLASAWSQEAAAIADEIKRLPHHHHHH

SEQ ID NO: 150 - RNA, 579 nt, Artificial Sequence
  Synthetic Polynucleotide
auggaaaccccagcccagcuccuguuccugcugcugcuguggcuucccgacaccaccggcgagaacaaggccuacuaccagcugcccaucgcccagagcggcgugcagagcacagccagccagggcaacagacugcuguggguggagcaggugagcgugcccgacuaccuggccggcaacggcgugguguaccagaccagcgacgugcaguacgugaucgccaacaacaaccugugggccagcccgcuggaccagcagcugagaaacacccugguggccaaccuggccgccagacuccccggcugggucguugccucccagccccugggcaccacccaggacacccugaacguggccgugaccggcuuccacggcagauacgacggcaaggugaucgugagcggcgaguggcugcugaaccacaacggccagcugaucaagaggcccuuccacaucgaggccagccagcagaaggacggcuacgacgagauggugaaggugcuggccagcgccuggagccaggaggccgcugccauagccgacgagaucaagagacugccccaccaucaccaccaccac

SEQ ID NO: 151 - protein, 30 aa, Artificial Sequence
  Synthetic Polypeptide
MDSKGSSQKGSRLLLLLVVSNLLLPQGVVG

SEQ ID NO: 152 - protein, 18 aa, Artificial Sequence
  Synthetic Polypeptide
MDWTWILFLVAAATRVHS

SEQ ID NO: 153 - protein, 20 aa, Artificial Sequence
  Synthetic Polypeptide
METPAQLLFLLLLWLPDTTG

SEQ ID NO: 154 - protein, 24 aa, Artificial Sequence
  Synthetic Polypeptide
MLGSNSGQRVVFTILLLLVAPAYS

SEQ ID NO: 155 - protein, 17 aa, Artificial Sequence
  Synthetic Polypeptide
MKCLLYLAFLFIGVNCA

SEQ ID NO: 156 - protein, 15 aa, Artificial Sequence
  Synthetic Polypeptide
MWLVSLAIVTACAGA

SEQ ID NO: 157 - protein, 273 aa, Artificial Sequence
  Synthetic Polypeptide
METPAQLLFLLLLWLPDTTGNISDYKVMTWNLQGSSASTESKWNVNVRQLLSGTAGVDILMVQEAGAVPTSAVPTGRHIQPFGVGIPIDEYTWNLGTTSRQDIRYIYHSAIDVGARRVNLAIVSRQRADNVYVLRPTTVASRPVIGIGLGNDVFLTAHALASGGPDAAAIVRVTINFFRQPQMRHLSWFLAGDFNRSPDRLENDLMTEHLERVVAVLAPTEPTQIGGGILDYGVIVDRAPYSQRVEALRNPQLASDHYPVAFLARSCHHHHHH

SEQ ID NO: 158 - RNA, 819 nt, Artificial Sequence
  Synthetic Polynucleotide
auggagacuccugcccagcugcuguuccugcugcugcuguggcugccugacaccaccggcaacaucagcgacuacaaggugaugaccuggaaccugcagggaagcuccgccagcaccgagagcaaguggaacgugaacgugagacagcuccugagcggcaccgccggcgucgacauccugauggugcaggaggccggagccgucccuaccagcgccgugccuaccggcagacacauccagccuuucggcgugggcaucccuaucgacgaguacaccuggaaucucggcaccaccagcagacaggacaucagauacaucuaccacagcgccaucgacgugggcgccagaagagugaaccuggccaucgugagcagacagagagccgacaacguguacgugcugaggccuaccaccguggccagcagaccugugaucggcaucggccugggcaacgacguguuccugaccgcccacgcucuggccuccgguggccccgacgcugccgccaucgugagagugaccaucaacuucuucagacagccucagaugagacaccugagcugguuccuggccggcgacuucaacagaagcccugacagacuggagaacgaccugaugaccgagcaccuggagagagugguggccgugcuggccccuaccgagccuacccagaucggcggcggcauccuggacuacggcgugaucguggacagagccccuuacagccagagaguggaggcccugagaaacccucagcuggccagcgaccacuacccuguggccuuccuggccagaagcugccaucaccaccaccaccac

SEQ ID NO: 159 - RNA, 9 nt, Artificial Sequence
  Synthetic Polynucleotide
ccrccaugg

SEQ ID NO: 160 - RNA, 9 nt, Artificial Sequence
  Synthetic Polynucleotide
uuauuuaww

SEQ ID NO: 161 - RNA, 1315 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcgccgagaucuacaacaaggacggcaacaagcuggaccuguucggcaagguggacggccugcacuacuucagcgacgauaagggcuccgacggcgaccagaccuacaugagaaucggcuucaagggcgagacacaagugaacgaccagcugacaggcuacggccagugggaguaucagauccagggcaaucagaccgagggcagcaacgacagcuggaccagaguggcuuuugccggccugaaguuugccgaugccggcagcuuugacuacggcagaaauuacggcgugaccuacgacgugaccuccuggacagaugugcugccugaguuuggcggcgauaccuacggcgccgacaacuucaugcagcagagaggcaacggcuacgccaccuaccggaacaccgauuucuucggccugguggauggccuggauuucgcccugcaguaccagggcaagaauggcucuguguccggcgagaacaccaacggcagaagccugcugaaccagaacggcgacggauauggcggcagccugacauaugccaucggcgagggcuuuucugucggcggagccaucaccaccagcaagagaacagccgaccagaacaacaccgccaacgccagacuguacggcaacggcgauagagccacaguguauaccggcggacugaaguacgacgccaacaacaucuaccuggccgcucaguacagccagacauacaacgccaccagauucggcaccagcaacggcagcaaucccagcacaagcuacggcuucgccaacaaggcccagaacuuugaggugguggcccaguaccaguucgacuucggacugaggccuagcguggccuaccugcagagcaagggcaaagacaucagcaacggauacggcgccagcuacggcgaucaggacaucgugaaauacguggacgugggcgccaccuauuacuucaacaagaacaugagcaccuacguggacuacaagaucaaccugcuggacaagaacgacuucacccgcgacgccggcaucaacaccgaugauauuguggcccugggccucguguaccaguuucaccaccaccaucaccauugauaauaggcuggagccucgguggccaugcuucuugccccuugggccuccccccagccccuccuccccuuccugcacccguacccccguggucuuugaauaaagucugagugggcggc

SEQ ID NO: 162 - RNA, 1297 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcgccgagaucuacaacaaggacggcaacaagcuggaccuguucggcaagguggacggccugcacuacuucagcgacgauaagggcuccgacggcgaccagaccuacaugagaaucggcuucaagggcgagacacaagugaacgaccagcugacaggcuacggccagugggaguaucagauccagggcaaucagaccgagggcagcaacgacagcuggaccagaguggcuuuugccggccugaaguuugccgaugccggcagcuuugacuacggcagaaauuacggcgugaccuacgacgugaccuccuggacagaugugcugccugaguuuggcggcgauaccuacggcgccgacaacuucaugcagcagagaggcaacggcuacgccaccuaccggaacaccgauuucuucggccugguggauggccuggauuucgcccugcaguaccagggcaagaauggcucuguguccggcgagaacaccaacggcagaagccugcugaaccagaacggcgacggauauggcggcagccugacauaugccaucggcgagggcuuuucugucggcggagccaucaccaccagcaagagaacagccgaccagaacaacaccgccaacgccagacuguacggcaacggcgauagagccacaguguauaccggcggacugaaguacgacgccaacaacaucuaccuggccgcucaguacagccagacauacaacgccaccagauucggcaccagcaacggcagcaaucccagcacaagcuacggcuucgccaacaaggcccagaacuuugaggugguggcccaguaccaguucgacuucggacugaggccuagcguggccuaccugcagagcaagggcaaagacaucagcaacggauacggcgccagcuacggcgaucaggacaucgugaaauacguggacgugggcgccaccuauuacuucaacaagaacaugagcaccuacguggacuacaagaucaaccugcuggacaagaacgacuucacccgcgacgccggcaucaacaccgaugauauuguggcccugggccucguguaccaguuuugauaauaggcuggagccucgguggccaugcuucuugccccuugggccuccccccagccccuccuccccuuccugcacccguacccccguggucuuugaauaaagucugagugggcggc

SEQ ID NO: 163 - RNA, 1267 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcgccgagaucuacaacaaggacggcaacaagcuggaccuguacggcaaagccgugggcagacacguguggacaaccaccggcgauagcaagaacgccgaccagacauaugcccagaucggcuucaagggcgagacacagaucaacaccgaccugaccggcuuuggccagugggaguacagaacaaaggccgacagagccgagggcgagcagcagaauucuaaucuugugcggcuggccuucgccggccugaaguaugcugaagugggcagcaucgacuacggccggaauuacggcaucguguacgacguggaaagcuacaccgacauggccccuuacuucagcggcgaaacauggggcggagccuacaccgauaacuacaugaccagcagagccggcggacugcugaccuacagaaacagcgauuucuucggccugguggacggccugagcuucggcauucaguaccagggcaagaaccaggacaaucacagcaucaacagccagaacggcgacggcgugggcuacacaauggccuacgaguucgauggcuuuggcgugacagccgccuacagcaacuccaagaggaccaacgaccagcaggacagagauggcaacggcgauagagccgaaucuugggccgugggcgccaaauacgacgccaacaauguguaucuggccgccguguacgccgagacacggaacaugagcaucguggagaacaccgugaccgacaccguggaaauggccaacaagacccagaaccuggaagugguggcccaguaccaguucgacuuuggacugaggcccgccaucagcuacgugcagucuaagggcaagcagcugaauggcgccgauggcucugcagaccuggccaaguauauucaggcuggcgccaccuacuacuucaacaagaacaugaacgugugggucgacuaccgguucaaccugcuggacgagaacgacuacagcagcuccuacgugggcaccgaugaucaggccgcugugggcaucaccuaccaguuccaccaccaccaucaccacugauaauaggcuggagccucgguggccaugcuucuugccccuugggccuccccccagccccuccuccccuuccugcacccguacccccguggucuuugaauaaagucugagugggcggc

SEQ ID NO: 164 - RNA, 1249 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcgccgagaucuacaacaaggacggcaacaagcuggaccuguacggcaaagccgugggcagacacguguggacaaccaccggcgauagcaagaacgccgaccagacauaugcccagaucggcuucaagggcgagacacagaucaacaccgaccugaccggcuuuggccagugggaguacagaacaaaggccgacagagccgagggcgagcagcagaauucuaaucuugugcggcuggccuucgccggccugaaguaugcugaagugggcagcaucgacuacggccggaauuacggcaucguguacgacguggaaagcuacaccgacauggccccuuacuucagcggcgaaacauggggcggagccuacaccgauaacuacaugaccagcagagccggcggacugcugaccuacagaaacagcgauuucuucggccugguggacggccugagcuucggcauucaguaccagggcaagaaccaggacaaucacagcaucaacagccagaacggcgacggcgugggcuacacaauggccuacgaguucgauggcuuuggcgugacagccgccuacagcaacuccaagaggaccaacgaccagcaggacagagauggcaacggcgauagagccgaaucuugggccgugggcgccaaauacgacgccaacaauguguaucuggccgccguguacgccgagacacggaacaugagcaucguggagaacaccgugaccgacaccguggaaauggccaacaagacccagaaccuggaagugguggcccaguaccaguucgacuuuggacugaggcccgccaucagcuacgugcagucuaagggcaagcagcugaauggcgccgauggcucugcagaccuggccaaguauauucaggcuggcgccaccuacuacuucaacaagaacaugaacgugugggucgacuaccgguucaaccugcuggacgagaacgacuacagcagcuccuacgugggcaccgaugaucaggccgcugugggcaucaccuaccaguucugauaauaggcuggagccucgguggccaugcuucuugccccuugggccuccccccagccccuccuccccuuccugcacccguacccccguggucuuugaauaaagucugagugggcggc

SEQ ID NO: 165 - RNA, 1267 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcgccgagaucuacaacaaggacggcaacaagcuggaccuguacggcaaagccgugggcagacacguguggacaaccaccggcgauagcaagaacgccgaccagacauaugcccagaucggcuucaagggcgagacacagaucaacaccgaccugaccggcuuuggccagugggaguacagaacaaaggccgacagagccgagggcgagcagcagaauucuaaucuugugcggcuggccuucgccggccugaaguaugcugaagugggcagcaucgacuacggccggaauuacggcaucguguacgacguggaaagcuacaccgacauggccccuuacuucagcggcgaaacauggggcggagccuacaccgauaacuacaugaccagcagagccggcggacugcugaccuacagaaacagcgauuucuucggccugguggacggccugagcuucggcauucaguaccagggcaagaaccaggacaaucacagcaucaacagccagaacggcgacggcgugggcuacacaauggccuacgaguucgauggcuuuggcgugacagccgccuacagcaacuccaagaggaccaacgaccagcaggacagagauggcaacggcgauagagccgaaucuugggccgugggcgccaaauacgacgccaacaauguguaucuggccgccguguacgccgagacaagaaacauggccaucguggagaacaccgugaccgacaccguggaaauggccaacaaggcccagaaccuggaagugguggcccaguaccaguucgacuuuggacugaggcccgccaucagcuacgugcagucuaagggcaagcagcugaauggcgccgauggcucugcagaccuggccaaguauauucaggcuggcgccaccuacuacuucaacaagaacaugaacgugugggucgacuaccgguucaaccugcuggacgagaacgacuacagcagcuccuacgugggcaccgaugaucaggccgcugugggcaucaccuaccaguuccaccaccaccaucaccacugauaauaggcuggagccucgguggccaugcuucuugccccuugggccuccccccagccccuccuccccuuccugcacccguacccccguggucuuugaauaaagucugagugggcggc

SEQ ID NO: 166 - RNA, 1249 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcgccgagaucuacaacaaggacggcaacaagcuggaccuguacggcaaagccgugggcagacacguguggacaaccaccggcgauagcaagaacgccgaccagacauaugcccagaucggcuucaagggcgagacacagaucaacaccgaccugaccggcuuuggccagugggaguacagaacaaaggccgacagagccgagggcgagcagcagaauucuaaucuugugcggcuggccuucgccggccugaaguaugcugaagugggcagcaucgacuacggccggaauuacggcaucguguacgacguggaaagcuacaccgacauggccccuuacuucagcggcgaaacauggggcggagccuacaccgauaacuacaugaccagcagagccggcggacugcugaccuacagaaacagcgauuucuucggccugguggacggccugagcuucggcauucaguaccagggcaagaaccaggacaaucacagcaucaacagccagaacggcgacggcgugggcuacacaauggccuacgaguucgauggcuuuggcgugacagccgccuacagcaacuccaagaggaccaacgaccagcaggacagagauggcaacggcgauagagccgaaucuugggccgugggcgccaaauacgacgccaacaauguguaucuggccgccguguacgccgagacaagaaacauggccaucguggagaacaccgugaccgacaccguggaaauggccaacaaggcccagaaccuggaagugguggcccaguaccaguucgacuuuggacugaggcccgccaucagcuacgugcagucuaagggcaagcagcugaauggcgccgauggcucugcagaccuggccaaguauauucaggcuggcgccaccuacuacuucaacaagaacaugaacgugugggucgacuaccgguucaaccugcuggacgagaacgacuacagcagcuccuacgugggcaccgaugaucaggccgcugugggcaucaccuaccaguucugauaauaggcuggagccucgguggccaugcuucuugccccuugggccuccccccagccccuccuccccuuccugcacccguacccccguggucuuugaauaaagucugagugggcggc

SEQ ID NO: 167 - RNA, 1267 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcgccgagguguacaacaaggacggcaacaagcuggaccuguacggcaaagugcacgcccagcacuacuucagcgacgacaauggcagcgacggcgacaagacauaugcccggcuuggcuucaagggcgagacacagaucaacgaccagcugaccggcuuuggccagugggaguacgaguucaagggcaacagaaccgagagccagggcgccgacaaggacaagaccagacuggccuuugccggccugaaguucgccgauuacggcagcuuugacuacggccggaauuacggcguggccuacgauaucggagccuggacagaugugcugccugaguuuggcggcgacaccuggacacagaccgacguguucaugaccggcagaacaacuggcguggccaccuaccggaacaccgauuucuuuggccugguggaaggccugaacuuugccgcucaguaccagggcaagaacgacagagauggcgccuacgagucuaacggcgacggcuuuggacugagcgccaccuacgaguacgaaggcuuuggagugggcgcugccuacgccaagagcgacaggaccaacaaucaagugaaggccgccagcaaccugaacgccgcuggaaagaaugccgaagugugggccgcuggacugaaguacgacgccaacaacaucuaccuggccaccaccuacagcgagacacugaacaugaccaccuucggcgaagaugccgcuggcgacgccuuuaucgccaacaagacccagaacuucgaggcuguggcccaguaccaguucgacuucggacugaggcccucuaucgccuaccugaaguccaagggaaagaaccugggcaccuacggcgaccaggaccugguugaguauaucgaugugggagccaccuacuacuucaacaagaauaugagcaccuucguggacuacaagaucaaccugcuggacgacagcgacuucaccaaagccgccaagguguccaccgacaacauuguggccgugggccugaauuaccaguuccaccaccaccaucaccacugauaauaggcuggagccucgguggccaugcuucuugccccuugggccuccccccagccccuccuccccuuccugcacccguacccccguggucuuugaauaaagucugagugggcggc

SEQ ID NO: 168 - RNA, 1249 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcgccgagguguacaacaaggacggcaacaagcuggaccuguacggcaaagugcacgcccagcacuacuucagcgacgacaauggcagcgacggcgacaagacauaugcccggcuuggcuucaagggcgagacacagaucaacgaccagcugaccggcuuuggccagugggaguacgaguucaagggcaacagaaccgagagccagggcgccgacaaggacaagaccagacuggccuuugccggccugaaguucgccgauuacggcagcuuugacuacggccggaauuacggcguggccuacgauaucggagccuggacagaugugcugccugaguuuggcggcgacaccuggacacagaccgacguguucaugaccggcagaacaacuggcguggccaccuaccggaacaccgauuucuuuggccugguggaaggccugaacuuugccgcucaguaccagggcaagaacgacagagauggcgccuacgagucuaacggcgacggcuuuggacugagcgccaccuacgaguacgaaggcuuuggagugggcgcugccuacgccaagagcgacaggaccaacaaucaagugaaggccgccagcaaccugaacgccgcuggaaagaaugccgaagugugggccgcuggacugaaguacgacgccaacaacaucuaccuggccaccaccuacagcgagacacugaacaugaccaccuucggcgaagaugccgcuggcgacgccuuuaucgccaacaagacccagaacuucgaggcuguggcccaguaccaguucgacuucggacugaggcccucuaucgccuaccugaaguccaagggaaagaaccugggcaccuacggcgaccaggaccugguugaguauaucgaugugggagccaccuacuacuucaacaagaauaugagcaccuucguggacuacaagaucaaccugcuggacgacagcgacuucaccaaagccgccaagguguccaccgacaacauuguggccgugggccugaauuaccaguucugauaauaggcuggagccucgguggccaugcuucuugccccuugggccuccccccagccccuccuccccuuccugcacccguacccccguggucuuugaauaaagucugagugggcggc

SEQ ID NO: 169 - RNA, 1315 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcgccgagaucuacaacaaggacggcaacaagcuggaccuguucggcaagguggacggccugcacuacuucagcgacgauaagggcuccgacggcgaccagaccuacaugagaaucggcuucaagggcgagacacaagugaacgaccagcugacaggcuacggccagugggaguaucagauccagggcaaucaggccgagggcagcaacgacgcauggaccagaguggcuuuugccggccugaaguuugccgaugccggcagcuuugacuacggcagaaauuacggcgugaccuacgacgugaccuccuggacagaugugcugccugaguuuggcggcgauaccuacggcgccgacaacuucaugcagcagagaggcaacggcuacgccaccuaccggaacaccgauuucuucggccugguggauggccuggauuucgcccugcaguaccagggcaagaauggcgcuguguccggcgagaacaccaacggcagaagccugcugaaccagaacggcgacggauauggcggcagccugacauaugccaucggcgagggcuuuucugucggcggagccaucaccaccagcaagagaacagccgaccagaacaacgccgccaacgccagacuguacggcaacggcgauagagccacaguguauaccggcggacugaaguacgacgccaacaacaucuaccuggccgcucaguacagccagacauacaacgccgccagauucggcaccagcaacggcgcaaaucccagcacaagcuacggcuucgccaacaaggcccagaacuuugaggugguggcccaguaccaguucgacuucggacugaggccuagcguggccuaccugcagagcaagggcaaagacaucagcaacggauacggcgccagcuacggcgaucaggacaucgugaaauacguggacgugggcgccaccuauuacuucaacaagaacaugagcaccuacguggacuacaagaucaaccugcuggacaagaacgacuucacccgcgacgccggcaucaacaccgaugauauuguggcccugggccucguguaccaguuucaccaccaccaucaccauugauaauaggcuggagccucgguggccaugcuucuugccccuugggccuccccccagccccuccuccccuuccugcacccguacccccguggucuuugaauaaagucugagugggcggc

SEQ ID NO: 170 - RNA, 1297 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcgccgagaucuacaacaaggacggcaacaagcuggaccuguucggcaagguggacggccugcacuacuucagcgacgauaagggcuccgacggcgaccagaccuacaugagaaucggcuucaagggcgagacacaagugaacgaccagcugacaggcuacggccagugggaguaucagauccagggcaaucaggccgagggcagcaacgacgcauggaccagaguggcuuuugccggccugaaguuugccgaugccggcagcuuugacuacggcagaaauuacggcgugaccuacgacgugaccuccuggacagaugugcugccugaguuuggcggcgauaccuacggcgccgacaacuucaugcagcagagaggcaacggcuacgccaccuaccggaacaccgauuucuucggccugguggauggccuggauuucgcccugcaguaccagggcaagaauggcgcuguguccggcgagaacaccaacggcagaagccugcugaaccagaacggcgacggauauggcggcagccugacauaugccaucggcgagggcuuuucugucggcggagccaucaccaccagcaagagaacagccgaccagaacaacgccgccaacgccagacuguacggcaacggcgauagagccacaguguauaccggcggacugaaguacgacgccaacaacaucuaccuggccgcucaguacagccagacauacaacgccgccagauucggcaccagcaacggcgcaaaucccagcacaagcuacggcuucgccaacaaggcccagaacuuugaggugguggcccaguaccaguucgacuucggacugaggccuagcguggccuaccugcagagcaagggcaaagacaucagcaacggauacggcgccagcuacggcgaucaggacaucgugaaauacguggacgugggcgccaccuauuacuucaacaagaacaugagcaccuacguggacuacaagaucaaccugcuggacaagaacgacuucacccgcgacgccggcaucaacaccgaugauauuguggcccugggccucguguaccaguuuugauaauaggcuggagccucgguggccaugcuucuugccccuugggccuccccccagccccuccuccccuuccugcacccguacccccguggucuuugaauaaagucugagugggcggc

SEQ ID NO: 171 - RNA, 967 nt, Artificial Sequence
  Synthetic Polynucleotide
gggaaauaagagagaaaagaagaguaagaagaaauauaagagccaccaugauccccaugagcgagacaaagaacgagcuggaaauccugcucgagaaggccgccacagagccugcucacagaagcgcuuucuucagaacccugcuggaaagcaccgugugggugccaggaucugcugcugaaggcgaagccaucguggaagauagcgcccuggaucugcagcacugggagaaagaggacggaaccacagucaucccauucuucaccagccuggaagcccugcagcaggcuguggaagaugagcaggccuucguggucaugcccgccagaacacuguucgagaugacccugggcgagacacuguuccugaacgccaaacugcccaccggcaaagaauucaugcccagagagaucucccugcugcuggccgaggaaggaucuccucugagcacacaagaggugcuggaaggcggcgagagccugauucugucugaaguggccgagccuccuagccagaugaucgacagccugaccacacuguucaagaccaucaagcccgugaagcgggccuuccugugcgccaucaaagaacacgcugacgcccagccuaaccugcugaucggaauugaggccgacggcgagaucgaggaaaucauccacgccgcuggaaacguggccaccgauacacugccuggcgacgagccuaucgacaucugccaaguucggaaaggcgcccagggaaucagccacuucaucaccgaacacauugccccauucuacgagcggagauggggcggcuuccugagagacuucaagcagaaccggaucauccaccaccaccaucaccacugauaauaggcuggagccucgguggccaugcuucuugccccuugggccuccccccagccccuccuccccuuccugcacccguacccccguggucuuugaauaaagucugagugggcggc

SEQ ID NO: 172 - RNA, 949 nt, Artificial Sequence
  Synthetic Polynucleotide
gggaaauaagagagaaaagaagaguaagaagaaauauaagagccaccaugauccccaugagcgagacaaagaacgagcuggaaauccugcucgagaaggccgccacagagccugcucacagaagcgcuuucuucagaacccugcuggaaagcaccgugugggugccaggaucugcugcugaaggcgaagccaucguggaagauagcgcccuggaucugcagcacugggagaaagaggacggaaccacagucaucccauucuucaccagccuggaagcccugcagcaggcuguggaagaugagcaggccuucguggucaugcccgccagaacacuguucgagaugacccugggcgagacacuguuccugaacgccaaacugcccaccggcaaagaauucaugcccagagagaucucccugcugcuggccgaggaaggaucuccucugagcacacaagaggugcuggaaggcggcgagagccugauucugucugaaguggccgagccuccuagccagaugaucgacagccugaccacacuguucaagaccaucaagcccgugaagcgggccuuccugugcgccaucaaagaacacgcugacgcccagccuaaccugcugaucggaauugaggccgacggcgagaucgaggaaaucauccacgccgcuggaaacguggccaccgauacacugccuggcgacgagccuaucgacaucugccaaguucggaaaggcgcccagggaaucagccacuucaucaccgaacacauugccccauucuacgagcggagauggggcggcuuccugagagacuucaagcagaaccggaucaucugauaauaggcuggagccucgguggccaugcuucuugccccuugggccuccccccagccccuccuccccuuccugcacccguacccccguggucuuugaauaaagucugagugggcggc

SEQ ID NO: 173 - RNA, 1024 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcauccccaugagcgagacaaagaacgagcuggaaauccugcucgagaaggccgccacagagccugcucacagaagcgcuuucuucagaacccugcuggaaagcaccgugugggugccaggaucugcugcugaaggcgaagccaucguggaagauagcgcccuggaucugcagcacugggagaaagaggacggaaccacagucaucccauucuucaccagccuggaagcccugcagcaggcuguggaagaugagcaggccuucguggucaugcccgccagaacacuguucgagaugacccugggcgagacacuguuccugaacgccaaacugcccaccggcaaagaauucaugcccagagagaucucccugcugcuggccgaggaaggaucuccucugagcacacaagaggugcuggaaggcggcgagagccugauucugucugaaguggccgagccuccuagccagaugaucgacagccugaccacacuguucaagaccaucaagcccgugaagcgggccuuccugugcgccaucaaagaacacgcugacgcccagccuaaccugcugaucggaauugaggccgacggcgagaucgaggaaaucauccacgccgcuggaaacguggccaccgauacacugccuggcgacgagccuaucgacaucugccaaguucggaaaggcgcccagggaaucagccacuucaucaccgaacacauugccccauucuacgagcggagauggggcggcuuccugagagacuucaagcagaaccggaucauccaccaccaccaucaccacugauaauaggcuggagccucgguggccaugcuucuugccccuugggccuccccccagccccuccuccccuuccugcacccguacccccguggucuuugaauaaagucugagugggcggc

SEQ ID NO: 174 - RNA, 1006 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcauccccaugagcgagacaaagaacgagcuggaaauccugcucgagaaggccgccacagagccugcucacagaagcgcuuucuucagaacccugcuggaaagcaccgugugggugccaggaucugcugcugaaggcgaagccaucguggaagauagcgcccuggaucugcagcacugggagaaagaggacggaaccacagucaucccauucuucaccagccuggaagcccugcagcaggcuguggaagaugagcaggccuucguggucaugcccgccagaacacuguucgagaugacccugggcgagacacuguuccugaacgccaaacugcccaccggcaaagaauucaugcccagagagaucucccugcugcuggccgaggaaggaucuccucugagcacacaagaggugcuggaaggcggcgagagccugauucugucugaaguggccgagccuccuagccagaugaucgacagccugaccacacuguucaagaccaucaagcccgugaagcgggccuuccugugcgccaucaaagaacacgcugacgcccagccuaaccugcugaucggaauugaggccgacggcgagaucgaggaaaucauccacgccgcuggaaacguggccaccgauacacugccuggcgacgagccuaucgacaucugccaaguucggaaaggcgcccagggaaucagccacuucaucaccgaacacauugccccauucuacgagcggagauggggcggcuuccugagagacuucaagcagaaccggaucaucugauaauaggcuggagccucgguggccaugcuucuugccccuugggccuccccccagccccuccuccccuuccugcacccguacccccguggucuuugaauaaagucugagugggcggc

SEQ ID NO: 175 - RNA, 1702 nt, Artificial Sequence
  Synthetic Polynucleotide
gggaaauaagagagaaaagaagaguaagaagaaauauaagagccaccauggcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcaaucugccaauggcacaaacagccaguccgaccuggauuccauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaaagagaucuccagcaagacccugggccucgacaagcugaacgugcaggaugccuacacacccaaagaaaccgccgugaccguggacaagaccaccuacaagaacggcacagaccccaucacagcccagagcaacaccgauauccagaccgccauuggaggcggagcuacagguguuacaggcgccgacaucaaguucaaggacggccaguacuaccuggacgugaaaggcggagcaucugccggcguguacaaggccacauacgacgaaaccaccaagaaagugaacaucgacaccaccgacaagaccccucuggccacagcugaagccacagccauuagaggcaccgccacaaucacccacaaccagaucgccgaagugaccaaagaaggcguggacaccacaaccguggcugcucaacuugcugcugcuggcguuaccggcgcugacaaggauaauaccagccuggucaagcugagcuucgaggacaagaauggcaaagugaucgacggcggcuacgccgugaagaugggcgacgauuucuacgccgccaccuacgaugagaagaccggcgccauuaccgccaagaccacaaccuacacagauggcacaggcguggcacagacaggggccgugaaauuuggaggcgccaacggcaagagcgaggucgugacagcuaccgacggcaagacauaccuggccagcgaucuggacaagcacaacuucagaacaggcggcgagcugaaagaagugaacacagacaagaccgagaauccgcugcagaagaucgacgcugcucuggcacaaguggacacccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugagacaccaccaccaucaccauugauaauaggcuggagccucgguggccaugcuucuugccccuugggccuccccccagccccuccuccccuuccugcacccguacccccguggucuuugaauaaagucugagugggcggc

SEQ ID NO: 176 - RNA, 1684 nt, Artificial Sequence
  Synthetic Polynucleotide
gggaaauaagagagaaaagaagaguaagaagaaauauaagagccaccauggcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcaaucugccaauggcacaaacagccaguccgaccuggauuccauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaaagagaucuccagcaagacccugggccucgacaagcugaacgugcaggaugccuacacacccaaagaaaccgccgugaccguggacaagaccaccuacaagaacggcacagaccccaucacagcccagagcaacaccgauauccagaccgccauuggaggcggagcuacagguguuacaggcgccgacaucaaguucaaggacggccaguacuaccuggacgugaaaggcggagcaucugccggcguguacaaggccacauacgacgaaaccaccaagaaagugaacaucgacaccaccgacaagaccccucuggccacagcugaagccacagccauuagaggcaccgccacaaucacccacaaccagaucgccgaagugaccaaagaaggcguggacaccacaaccguggcugcucaacuugcugcugcuggcguuaccggcgcugacaaggauaauaccagccuggucaagcugagcuucgaggacaagaauggcaaagugaucgacggcggcuacgccgugaagaugggcgacgauuucuacgccgccaccuacgaugagaagaccggcgccauuaccgccaagaccacaaccuacacagauggcacaggcguggcacagacaggggccgugaaauuuggaggcgccaacggcaagagcgaggucgugacagcuaccgacggcaagacauaccuggccagcgaucuggacaagcacaacuucagaacaggcggcgagcugaaagaagugaacacagacaagaccgagaauccgcugcagaagaucgacgcugcucuggcacaaguggacacccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugagaugauaauaggcuggagccucgguggccaugcuucuugccccuugggccuccccccagccccuccuccccuuccugcacccguacccccguggucuuugaauaaagucugagugggcggc

SEQ ID NO: 177 - RNA, 1759 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcgcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcaaucugccaauggcacaaacagccaguccgaccuggauuccauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaaagagaucuccagcaagacccugggccucgacaagcugaacgugcaggaugccuacacacccaaagaaaccgccgugaccguggacaagaccaccuacaagaacggcacagaccccaucacagcccagagcaacaccgauauccagaccgccauuggaggcggagcuacagguguuacaggcgccgacaucaaguucaaggacggccaguacuaccuggacgugaaaggcggagcaucugccggcguguacaaggccacauacgacgaaaccaccaagaaagugaacaucgacaccaccgacaagaccccucuggccacagcugaagccacagccauuagaggcaccgccacaaucacccacaaccagaucgccgaagugaccaaagaaggcguggacaccacaaccguggcugcucaacuugcugcugcuggcguuaccggcgcugacaaggauaauaccagccuggucaagcugagcuucgaggacaagaauggcaaagugaucgacggcggcuacgccgugaagaugggcgacgauuucuacgccgccaccuacgaugagaagaccggcgccauuaccgccaagaccacaaccuacacagauggcacaggcguggcacagacaggggccgugaaauuuggaggcgccaacggcaagagcgaggucgugacagcuaccgacggcaagacauaccuggccagcgaucuggacaagcacaacuucagaacaggcggcgagcugaaagaagugaacacagacaagaccgagaauccgcugcagaagaucgacgcugcucuggcacaaguggacacccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugagacaccaccaccaucaccauugauaauaggcuggagccucgguggccaugcuucuugccccuugggccuccccccagccccuccuccccuuccugcacccguacccccguggucuuugaauaaagucugagugggcggc

SEQ ID NO: 178 - RNA, 1741 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacaggcgcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcaaucugccaauggcacaaacagccaguccgaccuggauuccauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaaagagaucuccagcaagacccugggccucgacaagcugaacgugcaggaugccuacacacccaaagaaaccgccgugaccguggacaagaccaccuacaagaacggcacagaccccaucacagcccagagcaacaccgauauccagaccgccauuggaggcggagcuacagguguuacaggcgccgacaucaaguucaaggacggccaguacuaccuggacgugaaaggcggagcaucugccggcguguacaaggccacauacgacgaaaccaccaagaaagugaacaucgacaccaccgacaagaccccucuggccacagcugaagccacagccauuagaggcaccgccacaaucacccacaaccagaucgccgaagugaccaaagaaggcguggacaccacaaccguggcugcucaacuugcugcugcuggcguuaccggcgcugacaaggauaauaccagccuggucaagcugagcuucgaggacaagaauggcaaagugaucgacggcggcuacgccgugaagaugggcgacgauuucuacgccgccaccuacgaugagaagaccggcgccauuaccgccaagaccacaaccuacacagauggcacaggcguggcacagacaggggccgugaaauuuggaggcgccaacggcaagagcgaggucgugacagcuaccgacggcaagacauaccuggccagcgaucuggacaagcacaacuucagaacaggcggcgagcugaaagaagugaacacagacaagaccgagaauccgcugcagaagaucgacgcugcucuggcacaaguggacacccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugagaugauaauaggcuggagccucgguggccaugcuucuugccccuugggccuccccccagccccuccuccccuuccugcacccguacccccguggucuuugaauaaagucugagugggcggc

SEQ ID NO: 179 - RNA, 1669 nt, Artificial Sequence
  Synthetic Polynucleotide
gggaaauaagagagaaaagaagaguaagaagaaauauaagagccaccauggcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacaacggcgagacaaucgacaucgaccugaagcagaucaacucucagacccugggccucgacacccugaacgugcagaagaaguacgacgucaagagcgaggccgugacaccuagcgccacacugucuacaacagcccuggauggcgccggacucaagacaggcacaggcagcacaacagacaccggcuccaucaaggacggcaagguguacuacaacuccaccagcaagaacuacuacgucgagguggaauucaccgacgccaccgaccagacaaacaaaggcggcuucuacaaagugaacguggccgacgauggggccgugacuaugacagccgccacuaccaaagaggccaccacaccuacaggcaucaccgaagugacccaggugcagaaaccuguggcugccccugcugcuauucaggcccaacugacagcugcccaugugacaggcgccgauacagccgagauggucaagaugagcuacaccgacaagaacggcaagaccauugacggcggcuucggagugaaagugggcgccgacaucuaugccgccaccaagaacaaggauggcagcuucagcaucaauaccaccgaguacaccgauaaggacgggaacaccaagacagcccugaaccagcuuggcggagccgauggaaagaccgagguggugucuaucgauggcaagaccuacaacgccagcaaggccgcuggccacaacuuuaaggcucagccugaacuggccgaagcugccgcugcuaccacagagaauccucuggccaagaucgaugccgcucuggcacaaguggaugcccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugagacaccaucaccaccaccauugauaauaggcuggagccucgguggccaugcuucuugccccuugggccuccccccagccccuccuccccuuccugcacccguacccccguggucuuugaauaaagucugagugggcggc

SEQ ID NO: 180 - RNA, 1651 nt, Artificial Sequence
  Synthetic Polynucleotide
gggaaauaagagagaaaagaagaguaagaagaaauauaagagccaccauggcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacaacggcgagacaaucgacaucgaccugaagcagaucaacucucagacccugggccucgacacccugaacgugcagaagaaguacgacgucaagagcgaggccgugacaccuagcgccacacugucuacaacagcccuggauggcgccggacucaagacaggcacaggcagcacaacagacaccggcuccaucaaggacggcaagguguacuacaacuccaccagcaagaacuacuacgucgagguggaauucaccgacgccaccgaccagacaaacaaaggcggcuucuacaaagugaacguggccgacgauggggccgugacuaugacagccgccacuaccaaagaggccaccacaccuacaggcaucaccgaagugacccaggugcagaaaccuguggcugccccugcugcuauucaggcccaacugacagcugcccaugugacaggcgccgauacagccgagauggucaagaugagcuacaccgacaagaacggcaagaccauugacggcggcuucggagugaaagugggcgccgacaucuaugccgccaccaagaacaaggauggcagcuucagcaucaauaccaccgaguacaccgauaaggacgggaacaccaagacagcccugaaccagcuuggcggagccgauggaaagaccgagguggugucuaucgauggcaagaccuacaacgccagcaaggccgcuggccacaacuuuaaggcucagccugaacuggccgaagcugccgcugcuaccacagagaauccucuggccaagaucgaugccgcucuggcacaaguggaugcccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugagaugauaauaggcuggagccucgguggccaugcuucuugccccuugggccuccccccagccccuccuccccuuccugcacccguacccccguggucuuugaauaaagucugagugggcggc

SEQ ID NO: 181 - RNA, 1723 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacagcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacaacggcgagacaaucgacaucgaccugaagcagaucaacucucagacccugggccucgacacccugaacgugcagaagaaguacgacgucaagagcgaggccgugacaccuagcgccacacugucuacaacagcccuggauggcgccggacucaagacaggcacaggcagcacaacagacaccggcuccaucaaggacggcaagguguacuacaacuccaccagcaagaacuacuacgucgagguggaauucaccgacgccaccgaccagacaaacaaaggcggcuucuacaaagugaacguggccgacgauggggccgugacuaugacagccgccacuaccaaagaggccaccacaccuacaggcaucaccgaagugacccaggugcagaaaccuguggcugccccugcugcuauucaggcccaacugacagcugcccaugugacaggcgccgauacagccgagauggucaagaugagcuacaccgacaagaacggcaagaccauugacggcggcuucggagugaaagugggcgccgacaucuaugccgccaccaagaacaaggauggcagcuucagcaucaauaccaccgaguacaccgauaaggacgggaacaccaagacagcccugaaccagcuuggcggagccgauggaaagaccgagguggugucuaucgauggcaagaccuacaacgccagcaaggccgcuggccacaacuuuaaggcucagccugaacuggccgaagcugccgcugcuaccacagagaauccucuggccaagaucgaugccgcucuggcacaaguggaugcccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugagacaccaucaccaccaccauugauaauaggcuggagccucgguggccaugcuucuugccccuugggccuccccccagccccuccuccccuuccugcacccguacccccguggucuuugaauaaagucugagugggcggc

SEQ ID NO: 182 - RNA, 1705 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacagcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacaacggcgagacaaucgacaucgaccugaagcagaucaacucucagacccugggccucgacacccugaacgugcagaagaaguacgacgucaagagcgaggccgugacaccuagcgccacacugucuacaacagcccuggauggcgccggacucaagacaggcacaggcagcacaacagacaccggcuccaucaaggacggcaagguguacuacaacuccaccagcaagaacuacuacgucgagguggaauucaccgacgccaccgaccagacaaacaaaggcggcuucuacaaagugaacguggccgacgauggggccgugacuaugacagccgccacuaccaaagaggccaccacaccuacaggcaucaccgaagugacccaggugcagaaaccuguggcugccccugcugcuauucaggcccaacugacagcugcccaugugacaggcgccgauacagccgagauggucaagaugagcuacaccgacaagaacggcaagaccauugacggcggcuucggagugaaagugggcgccgacaucuaugccgccaccaagaacaaggauggcagcuucagcaucaauaccaccgaguacaccgauaaggacgggaacaccaagacagcccugaaccagcuuggcggagccgauggaaagaccgagguggugucuaucgauggcaagaccuacaacgccagcaaggccgcuggccacaacuuuaaggcucagccugaacuggccgaagcugccgcugcuaccacagagaauccucuggccaagaucgaugccgcucuggcacaaguggaugcccugagaagugaucugggcgccgugcagaacagguucaacuccgccaucaccaaccugggcaacaccgugaacaaucugagcagcgccagaagccggaucgaggacagcgauuaugccaccgagguguccaacaugagcagagcccagauucugcagcaggccggcacaucuguucuggcucaggcaaaucaggugccccagaacgugcugucccugcugagaugauaauaggcuggagccucgguggccaugcuucuugccccuugggccuccccccagccccuccuccccuuccugcacccguacccccguggucuuugaauaaagucugagugggcggc

SEQ ID NO: 183 - RNA, 1669 nt, Artificial Sequence
  Synthetic Polynucleotide
gggaaauaagagagaaaagaagaguaagaagaaauauaagagccaccauggcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaagcagaucaacucucagacccugggccucgacacccugaacgugcagcagaaguacaagguuuccgacaccgccgccaccgugacaggcuaugccgauacaacaaucgcccuggacaacagcaccuucaaggccucugccacaggccucggaggcaccgaucagaagauugacggcgaucugaaguucgacgacaccaccggcaaguacuacgccaaagugacagugacaggcggcacaggcaaggacggcuacuacgaaguguccguggacaagaccaacggcaaagugacucuggcuggcggagccaccucuccucuuacugguggacuuccugccaccgccaccgaggacgugaagaaugugcaggucgccaacgccgaucugaccgaagcuaaagccgcucugacagcugcuggcgugaccggaacagccucuguggucaagaugagcuacaccgacaacaacggcaagaccaucgacggcggacuggcagugaaagugggcgacgauuacuacagcgccacacagaacaaggauggcagcaucuccaucaauaccaccaaguacaccgccgacgauggcaccucuaagaccgcucugaacaaacucggcggagccgauggcaagaccgagguggugucuaucggcggcaagacauacgccgccucuaaggccgagggccacaacuuuaaagcccagccugaucuggcugaggccgcugccacuaccacagagaauccucugcagaagaucgacgccgcucuggcacaaguggauacccugagaagcgaucugggcgccgugcagaacagguucaauagcgccaucaccaaccugggcaacaccgugaacaaucugaccagcgccagaagccggaucgaggacagcgauuaugccacagagguguccaacaugagcagagcccagauccugcagcaggccggaacaucuguucuggcucaggccaaucaggugccccagaaugugcugucccugcugagacaccaucaccaccaccauugauaauaggcuggagccucgguggccaugcuucuugccccuugggccuccccccagccccuccuccccuuccugcacccguacccccguggucuuugaauaaagucugagugggcggc

SEQ ID NO: 184 - RNA, 1651 nt, Artificial Sequence
  Synthetic Polynucleotide
gggaaauaagagagaaaagaagaguaagaagaaauauaagagccaccauggcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaagcagaucaacucucagacccugggccucgacacccugaacgugcagcagaaguacaagguuuccgacaccgccgccaccgugacaggcuaugccgauacaacaaucgcccuggacaacagcaccuucaaggccucugccacaggccucggaggcaccgaucagaagauugacggcgaucugaaguucgacgacaccaccggcaaguacuacgccaaagugacagugacaggcggcacaggcaaggacggcuacuacgaaguguccguggacaagaccaacggcaaagugacucuggcuggcggagccaccucuccucuuacugguggacuuccugccaccgccaccgaggacgugaagaaugugcaggucgccaacgccgaucugaccgaagcuaaagccgcucugacagcugcuggcgugaccggaacagccucuguggucaagaugagcuacaccgacaacaacggcaagaccaucgacggcggacuggcagugaaagugggcgacgauuacuacagcgccacacagaacaaggauggcagcaucuccaucaauaccaccaaguacaccgccgacgauggcaccucuaagaccgcucugaacaaacucggcggagccgauggcaagaccgagguggugucuaucggcggcaagacauacgccgccucuaaggccgagggccacaacuuuaaagcccagccugaucuggcugaggccgcugccacuaccacagagaauccucugcagaagaucgacgccgcucuggcacaaguggauacccugagaagcgaucugggcgccgugcagaacagguucaauagcgccaucaccaaccugggcaacaccgugaacaaucugaccagcgccagaagccggaucgaggacagcgauuaugccacagagguguccaacaugagcagagcccagauccugcagcaggccggaacaucuguucuggcucaggccaaucaggugccccagaaugugcugucccugcugagaugauaauaggcuggagccucgguggccaugcuucuugccccuugggccuccccccagccccuccuccccuuccugcacccguacccccguggucuuugaauaaagucugagugggcggc

SEQ ID NO: 185 - RNA, 1723 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacagcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaagcagaucaacucucagacccugggccucgacacccugaacgugcagcagaaguacaagguuuccgacaccgccgccaccgugacaggcuaugccgauacaacaaucgcccuggacaacagcaccuucaaggccucugccacaggccucggaggcaccgaucagaagauugacggcgaucugaaguucgacgacaccaccggcaaguacuacgccaaagugacagugacaggcggcacaggcaaggacggcuacuacgaaguguccguggacaagaccaacggcaaagugacucuggcuggcggagccaccucuccucuuacugguggacuuccugccaccgccaccgaggacgugaagaaugugcaggucgccaacgccgaucugaccgaagcuaaagccgcucugacagcugcuggcgugaccggaacagccucuguggucaagaugagcuacaccgacaacaacggcaagaccaucgacggcggacuggcagugaaagugggcgacgauuacuacagcgccacacagaacaaggauggcagcaucuccaucaauaccaccaaguacaccgccgacgauggcaccucuaagaccgcucugaacaaacucggcggagccgauggcaagaccgagguggugucuaucggcggcaagacauacgccgccucuaaggccgagggccacaacuuuaaagcccagccugaucuggcugaggccgcugccacuaccacagagaauccucugcagaagaucgacgccgcucuggcacaaguggauacccugagaagcgaucugggcgccgugcagaacagguucaauagcgccaucaccaaccugggcaacaccgugaacaaucugaccagcgccagaagccggaucgaggacagcgauuaugccacagagguguccaacaugagcagagcccagauccugcagcaggccggaacaucuguucuggcucaggccaaucaggugccccagaaugugcugucccugcugagacaccaucaccaccaccauugauaauaggcuggagccucgguggccaugcuucuugccccuugggccuccccccagccccuccuccccuuccugcacccguacccccguggucuuugaauaaagucugagugggcggc

SEQ ID NO: 186 - RNA, 1705 nt, Artificial Sequence
  Synthetic Polynucleotide
gggaaauaagagagaaaagaagaguaagaagaaauauaagagccaccauggaaaccccugcucagcugcuguuccugcugcugcuguggcugccugauacaacagcccaagugaucaacaccaacagccugagccugcugacccagaacaaccugaacaagagccagagcgcccugggcacagccaucgaaagacugucuagcggccugcggaucaacagcgccaaagaugaugcugccggacaggccauugccaacagauucaccgccaacaucaagggccugacacaggccagcagaaacgccaacgacggcaucucuaucgcccagacaacagaaggcgcccugaacgagaucaacaacaaccugcagagagugcgcgagcuggccgugcagucugccaauagcacaaacagccaguccgaccuggacagcauccaggccgagaucacccagcggcugaaugagaucgacagaguguccggccagacacaguucaacggcgugaaagugcuggcccaggacaacacccugaccauccaagugggagccaacgauggcgagacaaucgacaucgaccugaagcagaucaacucucagacccugggccucgacacccugaacgugcagcagaaguacaagguuuccgacaccgccgccaccgugacaggcuaugccgauacaacaaucgcccuggacaacagcaccuucaaggccucugccacaggccucggaggcaccgaucagaagauugacggcgaucugaaguucgacgacaccaccggcaaguacuacgccaaagugacagugacaggcggcacaggcaaggacggcuacuacgaaguguccguggacaagaccaacggcaaagugacucuggcuggcggagccaccucuccucuuacugguggacuuccugccaccgccaccgaggacgugaagaaugugcaggucgccaacgccgaucugaccgaagcuaaagccgcucugacagcugcuggcgugaccggaacagccucuguggucaagaugagcuacaccgacaacaacggcaagaccaucgacggcggacuggcagugaaagugggcgacgauuacuacagcgccacacagaacaaggauggcagcaucuccaucaauaccaccaaguacaccgccgacgauggcaccucuaagaccgcucugaacaaacucggcggagccgauggcaagaccgagguggugucuaucggcggcaagacauacgccgccucuaaggccgagggccacaacuuuaaagcccagccugaucuggcugaggccgcugccacuaccacagagaauccucugcagaagaucgacgccgcucuggcacaaguggauacccugagaagcgaucugggcgccgugcagaacagguucaauagcgccaucaccaaccugggcaacaccgugaacaaucugaccagcgccagaagccggaucgaggacagcgauuaugccacagagguguccaacaugagcagagcccagauccugcagcaggccggaacaucuguucuggcucaggccaaucaggugccccagaaugugcugucccugcugagaugauaauaggcuggagccucgguggccaugcuucuugccccuugggccuccccccagccccuccuccccuuccugcacccguacccccguggucuuugaauaaagucugagugggcggc

SEQ ID NO: 187 - RNA, 1060 nt, Artificial Sequence
  Synthetic Polynucleotide
gggaaauaagagagaaaagaagaguaagaagaaauauaagagccaccaugaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccagcagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucagccgggcucgccacuacugccaggagagacagaagaagcuucucuucucaaucggaaucuuaaagccugagugggaguacaagagaauguggagcaccccuuacuucaccggcaagagcaucugcugauaauaggcuggagccucgguggccaugcuucuugccccuugggccuccccccagccccuccuccccuuccugcacccguacccccguggucuuugaauaaagucugagugggcggc

SEQ ID NO: 188 - RNA, 1078 nt, Artificial Sequence
  Synthetic Polynucleotide
gggaaauaagagagaaaagaagaguaagaagaaauauaagagccaccaugaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccagcagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucagccgggcucgccacuacugccaggagagacagaagaagcuucucuucucaaucggaaucuuaaagccugagugggaguacaagagaauguggagcaccccuuacuucaccggcaagagcaucugccaccaccaucaucaccacugauaauaggcuggagccucgguggccaugcuucuugccccuugggccuccccccagccccuccuccccuuccugcacccguacccccguggucuuugaauaaagucugagugggcggc

SEQ ID NO: 189 - RNA, 1117 nt, Artificial Sequence
  Synthetic Polynucleotide
gggaaauaagagagaaaagaagaguaagaagaaauauaagagccaccauggagacuccagcccagcuccuccuccuccucuugcucugguugccggacaccaccggcaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccagcagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucagccgggcucgccacuacugccaggagagacagaagaagcuucucuucucaaucggaaucuuaaagccugagugggaguacaagagaauguggagcaccccuuacuucaccggcaagagcaucugcugauaauaggcuggagccucgguggccaugcuucuugccccuugggccuccccccagccccuccuccccuuccugcacccguacccccguggucuuugaauaaagucugagugggcggc

SEQ ID NO: 190 - RNA, 1135 nt, Artificial Sequence
  Synthetic Polynucleotide
gggaaauaagagagaaaagaagaguaagaagaaauauaagagccaccauggagacuccagcccagcuccuccuccuccucuugcucugguugccggacaccaccggcaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccagcagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucagccgggcucgccacuacugccaggagagacagaagaagcuucucuucucaaucggaaucuuaaagccugagugggaguacaagagaauguggagcaccccuuacuucaccggcaagagcaucugccaccaccaucaucaccacugauaauaggcuggagccucgguggccaugcuucuugccccuugggccuccccccagccccuccuccccuuccugcacccguacccccguggucuuugaauaaagucugagugggcggc

SEQ ID NO: 191 - RNA, 1060 nt, Artificial Sequence
  Synthetic Polynucleotide
gggaaauaagagagaaaagaagaguaagaagaaauauaagagccaccaugaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccgccagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucgcccgggcucgccacuacugccaggagagacagaagaagcuucucuucucaaucggaaucuuaaagccugagugggaguacaagagaauguggagcaccccuuacuucaccggcaagagcaucugcugauaauaggcuggagccucgguggccaugcuucuugccccuugggccuccccccagccccuccuccccuuccugcacccguacccccguggucuuugaauaaagucugagugggcggc

SEQ ID NO: 192 - RNA, 1078 nt, Artificial Sequence
  Synthetic Polynucleotide
gggaaauaagagagaaaagaagaguaagaagaaauauaagagccaccaugaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccgccagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucgcccgggcucgccacuacugccaggagagacagaagaagcuucucuucucaaucggaaucuuaaagccugagugggaguacaagagaauguggagcaccccuuacuucaccggcaagagcaucugccaccaccaucaucaccacugauaauaggcuggagccucgguggccaugcuucuugccccuugggccuccccccagccccuccuccccuuccugcacccguacccccguggucuuugaauaaagucugagugggcggc

SEQ ID NO: 193 - RNA, 1117 nt, Artificial Sequence
  Synthetic Polynucleotide
gggaaauaagagagaaaagaagaguaagaagaaauauaagagccaccauggagacuccagcccagcuccuccuccuccucuugcucugguugccggacaccaccggcaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccgccagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucgcccgggcucgccacuacugccaggagagacagaagaagcuucucuucucaaucggaaucuuaaagccugagugggaguacaagagaauguggagcaccccuuacuucaccggcaagagcaucugcugauaauaggcuggagccucgguggccaugcuucuugccccuugggccuccccccagccccuccuccccuuccugcacccguacccccguggucuuugaauaaagucugagugggcggc

SEQ ID NO: 194 - RNA, 1135 nt, Artificial Sequence
  Synthetic Polynucleotide
gggaaauaagagagaaaagaagaguaagaagaaauauaagagccaccauggagacuccagcccagcuccuccuccuccucuugcucugguugccggacaccaccggcaagauccaggaggugaagagaauccucacccgauggcagccuagcagcuucacccuauacagagagguguucacccaguacggcggcagcaucaacaugcacccggacaucguggacuacuucaugaagagacacaacuggcacuucaaguucuuccacuacaaggaggacgacaagaucaagggcgcguauuucaucugcaacgaccagaacaucggcauccuaacacgaagaaccuucccucugagcagcgacgagauccugaucccuauggccccugaccugagaugcuuccugccugacagaaccaacagacugagcgcccugcaccagccucagaucagaaacgccaucuggaagcuggccagaaagaagcagaacugccuggugaaggaaacguucagcagcaaguucgagaagacucggcgcaacgaguaccagagauuccugaagaagggagguagcgucaagagcguggccgacugcaguucggacgaacugacccacaucuucaucgagcuguuccgauccagauucggcaacaccgccagcugcuacccugccgacaaccuggccaacuucuucagccagcugcaccaccugcuguucggccacauccuguacaucgagggcaucccuugcgccuucgacaucguuuugaagagugagagccagaugaacguguacuucgacgugagcaacggcgccaucaagaacgagugcagaccguuguccccuggaucuauccuuauguggcugaacaucgcccgggcucgccacuacugccaggagagacagaagaagcuucucuucucaaucggaaucuuaaagccugagugggaguacaagagaauguggagcaccccuuacuucaccggcaagagcaucugccaccaccaucaucaccacugauaauaggcuggagccucgguggccaugcuucuugccccuugggccuccccccagccccuccuccccuuccugcacccguacccccguggucuuugaauaaagucugagugggcggc

SEQ ID NO: 195 - RNA, 2392 nt, Artificial Sequence
  Synthetic Polynucleotide
gggaaauaagagagaaaagaagaguaagaagaaauauaagagccaccaugggccugcagaccacaaaguggccuucucacggcgcauucuuucugaaguccuggcugaucaucucccugggccuguacagccagguguccaaacugcuggccgccgagagcaccgacgauaacggcgagacaaucgugguggaaagcaccgccgaacaggugcugaaacagcagccuggcguguccaucaucacccgggacgacauccagaagaacccuccagucaacgaccuggccgacaucaucagaaagaugcccggcgugaaccugaccagcaauagcgccucuggcacccggggcaacaacagacagaucgacaucagaggcaugggccccgagaauacccuggugcugauugauggcgugcccgugaccagcagaaacagcgugcgguauucuuggagaggcgagagagacaccagaggcgacaccaauugggugccaccugagaugguggaacggaucgagaugauuagaggccccgcugccgccagauauggaucuggugcugcuggcggcguggucaauaucaucaccaagaggcccaccaacgacuggcacggcagccugagccuguauacaaacuaccccgagagcagcaaagagggcgauaccagaagaggcaacuuuagccugucuggcccucuggccggcgauacccugacaaugagacuguacggcaaccugaaccggaccgacgccgauagcugggacaucaauagcagcgccggcaccaagaaugccgccggaagagaaggcgugaccaacaaggacaucaacagcguguucagcuggaagaugaccccucagcagauccuggauuucgaggccggcuauagcagacagggcaacaucuaugccggcgacacccagaacagcaacagcaacgccgugaccaagucucuggcccagucuggcagagagacaaacaggcuguaccggcagaacuacggccugacacacaauggcaucuggggcuggggacagucuaggcugggcuucuacuacgagaagaccgacaacacccggaugaacgagggacuuucuggcggcggagagggcagaaucaccaacgaucagaccuucaccaccaaccggcugaccagcuacagaaccagcggcgaagugaacgugccagugaucuggcuguucgagcagacccugacagugggcgccgaguggaauagagaugagcugaacgaccccagcuccaccagccugaccgugaaggacucuaauaucgcuggcaucccuggcagcgccgccaacagaagcagcaagaacaagagcgagaucagcgcccuguacgucgaggacaacaucgaaccuauggccggcacaaacaucauccccggccugagauucgacuaccugagcgagagcggcagcaacuucagccccagccugaaucugucucaagagcugggcgaguucgugaaagugaaggccggaaucgccagagccuucaaggccccuaaucuguaccagaccagcgagggcuaccugcuguacuccaaaggcaacggcugccccaaggauaucacaagcggcggcuguuaccucgugggcaacaagaaccuggauccugagaucagcaucaacaaagagaucggccuggaauucaccguggacgacuaccacgccagcgugaccuacuuccggaacgauuaccagaacaagaucguggccggggaccagaucaucggcagaagugcuagcggagccuacgugcugcaauggcagaauggcggaaaggcccugaucgagggaaucgaggcuucuauggccgugccacugaugcccgacagacugaacuggaacaccaacgccaccuacaugaucaccagcgagcagaaggacaccggcaauccucugagcaucaucccuaaguauaccgugaacaccuuccuggacuggaccaucacaaacgcccugagcgccaacgugaacuggacccuguauggcaagcagaagccacggacacacgccgaguccagaagcgaggaaacaaagggccugagcggcaaagcccugggcgccuauucucuugugggcgccaaugugaauuacgacauuaacaagaaucugcggcugaacgugggcaucagcaacaucuucgacaagcagaucuacagaagcgccgagggcgccaacaccuacaaugaaccuggcagagccuacuaugcuggcgugaccgccagcuuccaucaccaccaucaucauugauaauaggcuggagccucgguggccaugcuucuugccccuugggccuccccccagccccuccuccccuuccugcacccguacccccguggucuuugaauaaagucugagugggcggc

SEQ ID NO: 196 - RNA, 2374 nt, Artificial Sequence
  Synthetic Polynucleotide
gggaaauaagagagaaaagaagaguaagaagaaauauaagagccaccaugggccugcagaccacaaaguggccuucucacggcgcauucuuucugaaguccuggcugaucaucucccugggccuguacagccagguguccaaacugcuggccgccgagagcaccgacgauaacggcgagacaaucgugguggaaagcaccgccgaacaggugcugaaacagcagccuggcguguccaucaucacccgggacgacauccagaagaacccuccagucaacgaccuggccgacaucaucagaaagaugcccggcgugaaccugaccagcaauagcgccucuggcacccggggcaacaacagacagaucgacaucagaggcaugggccccgagaauacccuggugcugauugauggcgugcccgugaccagcagaaacagcgugcgguauucuuggagaggcgagagagacaccagaggcgacaccaauugggugccaccugagaugguggaacggaucgagaugauuagaggccccgcugccgccagauauggaucuggugcugcuggcggcguggucaauaucaucaccaagaggcccaccaacgacuggcacggcagccugagccuguauacaaacuaccccgagagcagcaaagagggcgauaccagaagaggcaacuuuagccugucuggcccucuggccggcgauacccugacaaugagacuguacggcaaccugaaccggaccgacgccgauagcugggacaucaauagcagcgccggcaccaagaaugccgccggaagagaaggcgugaccaacaaggacaucaacagcguguucagcuggaagaugaccccucagcagauccuggauuucgaggccggcuauagcagacagggcaacaucuaugccggcgacacccagaacagcaacagcaacgccgugaccaagucucuggcccagucuggcagagagacaaacaggcuguaccggcagaacuacggccugacacacaauggcaucuggggcuggggacagucuaggcugggcuucuacuacgagaagaccgacaacacccggaugaacgagggacuuucuggcggcggagagggcagaaucaccaacgaucagaccuucaccaccaaccggcugaccagcuacagaaccagcggcgaagugaacgugccagugaucuggcuguucgagcagacccugacagugggcgccgaguggaauagagaugagcugaacgaccccagcuccaccagccugaccgugaaggacucuaauaucgcuggcaucccuggcagcgccgccaacagaagcagcaagaacaagagcgagaucagcgcccuguacgucgaggacaacaucgaaccuauggccggcacaaacaucauccccggccugagauucgacuaccugagcgagagcggcagcaacuucagccccagccugaaucugucucaagagcugggcgaguucgugaaagugaaggccggaaucgccagagccuucaaggccccuaaucuguaccagaccagcgagggcuaccugcuguacuccaaaggcaacggcugccccaaggauaucacaagcggcggcuguuaccucgugggcaacaagaaccuggauccugagaucagcaucaacaaagagaucggccuggaauucaccguggacgacuaccacgccagcgugaccuacuuccggaacgauuaccagaacaagaucguggccggggaccagaucaucggcagaagugcuagcggagccuacgugcugcaauggcagaauggcggaaaggcccugaucgagggaaucgaggcuucuauggccgugccacugaugcccgacagacugaacuggaacaccaacgccaccuacaugaucaccagcgagcagaaggacaccggcaauccucugagcaucaucccuaaguauaccgugaacaccuuccuggacuggaccaucacaaacgcccugagcgccaacgugaacuggacccuguauggcaagcagaagccacggacacacgccgaguccagaagcgaggaaacaaagggccugagcggcaaagcccugggcgccuauucucuugugggcgccaaugugaauuacgacauuaacaagaaucugcggcugaacgugggcaucagcaacaucuucgacaagcagaucuacagaagcgccgagggcgccaacaccuacaaugaaccuggcagagccuacuaugcuggcgugaccgccagcuucugauaauaggcuggagccucgguggccaugcuucuugccccuugggccuccccccagccccuccuccccuuccugcacccguacccccguggucuuugaauaaagucugagugggcggc

SEQ ID NO: 197 - RNA, 2392 nt, Artificial Sequence
  Synthetic Polynucleotide
gggaaauaagagagaaaagaagaguaagaagaaauauaagagccaccaugggccugcagaccacaaaguggccuucucacggcgcauucuuucugaaguccuggcugaucaucucccugggccuguacagccagguguccaaacugcuggccgccgagagcaccgacgauaacggcgagacaaucgugguggaaagcaccgccgaacaggugcugaaacagcagccuggcguguccaucaucacccgggacgacauccagaagaacccuccagucaacgaccuggccgacaucaucagaaagaugcccggcgugaaccuggccagcaauagcgccucuggcacccggggcaacaacagacagaucgacaucagaggcaugggccccgagaauacccuggugcugauugauggcgugcccgugaccagcagaaacagcgugcgguauucuuggagaggcgagagagacaccagaggcgacaccaauugggugccaccugagaugguggaacggaucgagaugauuagaggccccgcugccgccagauauggaucuggugcugcuggcggcguggucaauaucaucaccaagaggcccaccaacgacuggcacggcagccugagccuguauacaaacuaccccgagagcagcaaagagggcgauaccagaagaggcaacuuugcccugucuggcccucuggccggcgauacccugacaaugagacuguacggcaaccugaaccgggccgacgccgauagcugggacaucaauagcagcgccggcaccaagaaugccgccggaagagaaggcgugaccaacaaggacaucaacagcguguucagcuggaagaugaccccucagcagauccuggauuucgaggccggcuauagcagacagggcaacaucuaugccggcgacacccagaacagcaacagcaacgccgugaccaagucucuggcccagucuggcagagagacaaacaggcuguaccggcagaacuacggccugacacacaauggcaucuggggcuggggacagucuaggcugggcuucuacuacgagaagaccgacaacacccggaugaacgagggacuuucuggcggcggagagggcagaaucaccaacgaucagaccuucaccaccaaccggcugaccagcuacagaaccagcggcgaagugaacgugccagugaucuggcuguucgagcagacccugacagugggcgccgaguggaauagagaugagcugaacgaccccagcuccaccagccugaccgugaaggacucuaauaucgcuggcaucccuggcagcgccgccaacagagccagcaagaacaagagcgagaucagcgcccuguacgucgaggacaacaucgaaccuauggccggcacaaacaucauccccggccugagauucgacuaccugagcgagagcggcagcaacuucagccccagccugaaucuggcucaagagcugggcgaguucgugaaagugaaggccggaaucgccagagccuucaaggccccuaaucuguaccagaccagcgagggcuaccugcuguacuccaaaggcaacggcugccccaaggauaucacaagcggcggcuguuaccucgugggcaacaagaaccuggauccugagaucagcaucaacaaagagaucggccuggaauucaccguggacgacuaccacgccagcgugaccuacuuccggaacgauuaccagaacaagaucguggccggggaccagaucaucggcagaagugcuagcggagccuacgugcugcaauggcagaauggcggaaaggcccugaucgagggaaucgaggcuucuauggccgugccacugaugcccgacagacugaacuggaacaccaacgccgccuacaugaucaccagcgagcagaaggacaccggcaauccucugagcaucaucccuaaguauaccgugaacaccuuccuggacuggaccaucacaaacgcccugagcgccaacgugaacuggacccuguauggcaagcagaagccacggacacacgccgaguccagaagcgaggaaacaaagggccugagcggcaaagcccugggcgccuauucucuugugggcgccaaugugaauuacgacauuaacaagaaucugcggcugaacgugggcaucagcaacaucuucgacaagcagaucuacagaagcgccgagggcgccaacaccuacaaugaaccuggcagagccuacuaugcuggcgugaccgccagcuuccaucaccaccaucaucauugauaauaggcuggagccucgguggccaugcuucuugccccuugggccuccccccagccccuccuccccuuccugcacccguacccccguggucuuugaauaaagucugagugggcggc

SEQ ID NO: 198 - RNA, 2374 nt, Artificial Sequence
  Synthetic Polynucleotide
gggaaauaagagagaaaagaagaguaagaagaaauauaagagccaccaugggccugcagaccacaaaguggccuucucacggcgcauucuuucugaaguccuggcugaucaucucccugggccuguacagccagguguccaaacugcuggccgccgagagcaccgacgauaacggcgagacaaucgugguggaaagcaccgccgaacaggugcugaaacagcagccuggcguguccaucaucacccgggacgacauccagaagaacccuccagucaacgaccuggccgacaucaucagaaagaugcccggcgugaaccuggccagcaauagcgccucuggcacccggggcaacaacagacagaucgacaucagaggcaugggccccgagaauacccuggugcugauugauggcgugcccgugaccagcagaaacagcgugcgguauucuuggagaggcgagagagacaccagaggcgacaccaauugggugccaccugagaugguggaacggaucgagaugauuagaggccccgcugccgccagauauggaucuggugcugcuggcggcguggucaauaucaucaccaagaggcccaccaacgacuggcacggcagccugagccuguauacaaacuaccccgagagcagcaaagagggcgauaccagaagaggcaacuuugcccugucuggcccucuggccggcgauacccugacaaugagacuguacggcaaccugaaccgggccgacgccgauagcugggacaucaauagcagcgccggcaccaagaaugccgccggaagagaaggcgugaccaacaaggacaucaacagcguguucagcuggaagaugaccccucagcagauccuggauuucgaggccggcuauagcagacagggcaacaucuaugccggcgacacccagaacagcaacagcaacgccgugaccaagucucuggcccagucuggcagagagacaaacaggcuguaccggcagaacuacggccugacacacaauggcaucuggggcuggggacagucuaggcugggcuucuacuacgagaagaccgacaacacccggaugaacgagggacuuucuggcggcggagagggcagaaucaccaacgaucagaccuucaccaccaaccggcugaccagcuacagaaccagcggcgaagugaacgugccagugaucuggcuguucgagcagacccugacagugggcgccgaguggaauagagaugagcugaacgaccccagcuccaccagccugaccgugaaggacucuaauaucgcuggcaucccuggcagcgccgccaacagagccagcaagaacaagagcgagaucagcgcccuguacgucgaggacaacaucgaaccuauggccggcacaaacaucauccccggccugagauucgacuaccugagcgagagcggcagcaacuucagccccagccugaaucuggcucaagagcugggcgaguucgugaaagugaaggccggaaucgccagagccuucaaggccccuaaucuguaccagaccagcgagggcuaccugcuguacuccaaaggcaacggcugccccaaggauaucacaagcggcggcuguuaccucgugggcaacaagaaccuggauccugagaucagcaucaacaaagagaucggccuggaauucaccguggacgacuaccacgccagcgugaccuacuuccggaacgauuaccagaacaagaucguggccggggaccagaucaucggcagaagugcuagcggagccuacgugcugcaauggcagaauggcggaaaggcccugaucgagggaaucgaggcuucuauggccgugccacugaugcccgacagacugaacuggaacaccaacgccgccuacaugaucaccagcgagcagaaggacaccggcaauccucugagcaucaucccuaaguauaccgugaacaccuuccuggacuggaccaucacaaacgcccugagcgccaacgugaacuggacccuguauggcaagcagaagccacggacacacgccgaguccagaagcgaggaaacaaagggccugagcggcaaagcccugggcgccuauucucuugugggcgccaaugugaauuacgacauuaacaagaaucugcggcugaacgugggcaucagcaacaucuucgacaagcagaucuacagaagcgccgagggcgccaacaccuacaaugaaccuggcagagccuacuaugcuggcgugaccgccagcuucugauaauaggcuggagccucgguggccaugcuucuugccccuugggccuccccccagccccuccuccccuuccugcacccguacccccguggucuuugaauaaagucugagugggcggc

SEQ ID NO: 199 - RNA, 2203 nt, Artificial Sequence
  Synthetic Polynucleotide
gggaaauaagagagaaaagaagaguaagaagaaauauaagagccaccaugggccugcagaccacaaaguggccuucucacggcgcauucuuucugaaguccuggcugaucaucucccugggccuguacagccagguguccaaacugcuggccgccaccgaugauggcgagacaauggugguuacagccagcgccaucgagcagaaccugaaagaugccccugccagcaucagcgugaucacccagcaggaucugcagagaaggcccgugcagaaucugaaggacgugcugaaagaggugcccggcguccagcugacaaacgagggcgauaauagaaagggcguguccaucagaggccuggacagcagcuacacccugauccugaucgacggcaagagagugaacagccggaacgccguguuccggcacaacgacuucgaccugaacuggauccccguggaugccaucgagaggaucgaaguugugcggggaccuaugagcagccuguacggaucugaugcccucggcggcguggucaacaucaucaccaagaagaucggccagaaauggcacggcagcgugaccguggauagcaccauccaagagcacagagacagaggcgacaccuacaacggccaguucuucacaagcggcccucugauugauggcgugcugggcaugaaggccuacggaucucuggccaagagagagaaggacgagcagcagagcagcgccacaacagccacaggcgagacaccuagaaucgagggcuucaccagcagagauggcaacguggaauucgccuggacacccaacgagaaccacgaugugacagccggcuacggcuucgacagacaggacagagauagcgacagccuggacaagaaccggcuggaaagacagaacuacgcccugagccacaacggcagaugggaccugggaaacagcgagcugaaguucuacggcgagaaggucgagaacaagaaccccggcaacagcagcccuaucaccagcgagagcaacagcaucgauggcaaauacgugcugccccuggccuccgugaaucaguuccugacauuuggcggagaguggcggcacgacaagcugucugaugccguuucucugaccggcggcagcagcacaaagacaagcgccucucaguacgcccuguuccuggaagaugaguggcggaucuucgagccccuggcucugacaacaggcaucagaauggacgaccacgagacauacggcgaccacugguccccaagagccuaccugguguacaacgccaccgacacacugaccgugaaaggcggaugggccacagccuuuaaggcuccuagucugcugcagcugagccccgauugggccaccaauucuuguagaggcggcugcagaaucgugggcagcccugaucugaagcccgagacaucugagucuugggagcugggacuguacuacaggggcgaagagggaauccuggaaggcguggaagccagcgugacaaccuucagaaacgacguggacaaccggaucagcaucuccagaacacccgacgugaacgccgcuccuggcuacucuaauuucgugggcuucgagacaaacagcagaggccagagggugcccguguuucgguacuacaacgugaacaaggcccggauccagggcgucgagacagagcugaaggugcccuuuaacgaagccuggaagcugagccugaacuacacauacaacgacggccgggacguguccaacggcggaaacaaaccucugagcgaccugccuuuccacaccgccaauggcacccuggauuggaagccugcucagcuggaagauuggagcuucuacguguccggcaacuacaccggcagaaagagagccgauagcgccaccgcuaagacaccaggcggauacgucgugugggauacaggugcugcuuggcaggccaccaagaacgugaaacugagagccggcgugcugaacgugggcgacaaagaccugaagagggacgacuacggcuacaccgaggacggcagacgguacuucauggccguggacuaccgguuccaccaccaccaucaccauugauaauaggcuggagccucgguggccaugcuucuugccccuugggccuccccccagccccuccuccccuuccugcacccguacccccguggucuuugaauaaagucugagugggcggc

SEQ ID NO: 200 - RNA, 2185 nt, Artificial Sequence
  Synthetic Polynucleotide
gggaaauaagagagaaaagaagaguaagaagaaauauaagagccaccaugggccugcagaccacaaaguggccuucucacggcgcauucuuucugaaguccuggcugaucaucucccugggccuguacagccagguguccaaacugcuggccgccaccgaugauggcgagacaauggugguuacagccagcgccaucgagcagaaccugaaagaugccccugccagcaucagcgugaucacccagcaggaucugcagagaaggcccgugcagaaucugaaggacgugcugaaagaggugcccggcguccagcugacaaacgagggcgauaauagaaagggcguguccaucagaggccuggacagcagcuacacccugauccugaucgacggcaagagagugaacagccggaacgccguguuccggcacaacgacuucgaccugaacuggauccccguggaugccaucgagaggaucgaaguugugcggggaccuaugagcagccuguacggaucugaugcccucggcggcguggucaacaucaucaccaagaagaucggccagaaauggcacggcagcgugaccguggauagcaccauccaagagcacagagacagaggcgacaccuacaacggccaguucuucacaagcggcccucugauugauggcgugcugggcaugaaggccuacggaucucuggccaagagagagaaggacgagcagcagagcagcgccacaacagccacaggcgagacaccuagaaucgagggcuucaccagcagagauggcaacguggaauucgccuggacacccaacgagaaccacgaugugacagccggcuacggcuucgacagacaggacagagauagcgacagccuggacaagaaccggcuggaaagacagaacuacgcccugagccacaacggcagaugggaccugggaaacagcgagcugaaguucuacggcgagaaggucgagaacaagaaccccggcaacagcagcccuaucaccagcgagagcaacagcaucgauggcaaauacgugcugccccuggccuccgugaaucaguuccugacauuuggcggagaguggcggcacgacaagcugucugaugccguuucucugaccggcggcagcagcacaaagacaagcgccucucaguacgcccuguuccuggaagaugaguggcggaucuucgagccccuggcucugacaacaggcaucagaauggacgaccacgagacauacggcgaccacugguccccaagagccuaccugguguacaacgccaccgacacacugaccgugaaaggcggaugggccacagccuuuaaggcuccuagucugcugcagcugagccccgauugggccaccaauucuuguagaggcggcugcagaaucgugggcagcccugaucugaagcccgagacaucugagucuugggagcugggacuguacuacaggggcgaagagggaauccuggaaggcguggaagccagcgugacaaccuucagaaacgacguggacaaccggaucagcaucuccagaacacccgacgugaacgccgcuccuggcuacucuaauuucgugggcuucgagacaaacagcagaggccagagggugcccguguuucgguacuacaacgugaacaaggcccggauccagggcgucgagacagagcugaaggugcccuuuaacgaagccuggaagcugagccugaacuacacauacaacgacggccgggacguguccaacggcggaaacaaaccucugagcgaccugccuuuccacaccgccaauggcacccuggauuggaagccugcucagcuggaagauuggagcuucuacguguccggcaacuacaccggcagaaagagagccgauagcgccaccgcuaagacaccaggcggauacgucgugugggauacaggugcugcuuggcaggccaccaagaacgugaaacugagagccggcgugcugaacgugggcgacaaagaccugaagagggacgacuacggcuacaccgaggacggcagacgguacuucauggccguggacuaccgguucugauaauaggcuggagccucgguggccaugcuucuugccccuugggccuccccccagccccuccuccccuuccugcacccguacccccguggucuuugaauaaagucugagugggcggc

SEQ ID NO: 201 - RNA, 2203 nt, Artificial Sequence
  Synthetic Polynucleotide
gggaaauaagagagaaaagaagaguaagaagaaauauaagagccaccaugggccugcagaccacaaaguggccuucucacggcgcauucuuucugaaguccuggcugaucaucucccugggccuguacagccagguguccaaacugcuggccgccaccgaugauggcgagacaauggugguuacagccagcgccaucgagcagaaccugaaagaugccccugccagcaucagcgugaucacccagcaggaucugcagagaaggcccgugcagaaucugaaggacgugcugaaagaggugcccggcguccagcugacaaacgagggcgauaauagaaagggcguguccaucagaggccuggacagcagcuacacccugauccugaucgacggcaagagagugaacagccggaacgccguguuccggcacaacgacuucgaccugaacuggauccccguggaugccaucgagaggaucgaaguugugcggggaccuaugagcagccuguacggaucugaugcccucggcggcguggucaacaucaucaccaagaagaucggccagaaauggcacggcagcgugaccguggauagcaccauccaagagcacagagacagaggcgacaccuacaacggccaguucuucacaagcggcccucugauugauggcgugcugggcaugaaggccuacggaucucuggccaagagagagaaggacgagcagcagagcagcgccacaacagccacaggcgagacaccuagaaucgagggcuucaccagcagagauggcaacguggaauucgccuggacacccaacgagaaccacgaugugacagccggcuacggcuucgacagacaggacagagauagcgacagccuggacaagaaccggcuggaaagacagaacuacgcccugagccacaacggcagaugggaccugggaaacagcgagcugaaguucuacggcgagaaggucgagaacaagaaccccggcaacagcagcccuaucaccagcgagagcaacagcaucgauggcaaauacgugcugccccuggccuccgugaaucaguuccugacauuuggcggagaguggcggcacgacaagcugucugaugccguuucucugaccggcggcagcagcacaaagacaagcgccucucaguacgcccuguuccuggaagaugaguggcggaucuucgagccccuggcucugacaacaggcaucagaauggacgaccacgagacauacggcgaccacugguccccaagagccuaccugguguacaacgccgccgacacacugaccgugaaaggcggaugggccacagccuuuaaggcuccuagucugcugcagcugagccccgauugggccaccaauucuuguagaggcggcugcagaaucgugggcagcccugaucugaagcccgagacaucugagucuugggagcugggacuguacuacaggggcgaagagggaauccuggaaggcguggaagccagcgugacaaccuucagaaacgacguggacaaccggaucagcaucuccagaacacccgacgugaacgccgcuccuggcuacucuaauuucgugggcuucgagacaaacagcagaggccagagggugcccguguuucgguacuacaacgugaacaaggcccggauccagggcgucgagacagagcugaaggugcccuuuaacgaagccuggaagcugagccugaacuacgcauacaacgacggccgggacguguccaacggcggaaacaaaccucugagcgaccugccuuuccacaccgccaauggcgcccuggauuggaagccugcucagcuggaagauuggagcuucuacguguccggcaacuacgccggcagaaagagagccgauagcgccaccgcuaagacaccaggcggauacgucgugugggauacaggugcugcuuggcaggccaccaagaacgugaaacugagagccggcgugcugaacgugggcgacaaagaccugaagagggacgacuacggcuacaccgaggacggcagacgguacuucauggccguggacuaccgguuccaccaccaccaucaccauugauaauaggcuggagccucgguggccaugcuucuugccccuugggccuccccccagccccuccuccccuuccugcacccguacccccguggucuuugaauaaagucugagugggcggc

SEQ ID NO: 202 - RNA, 2185 nt, Artificial Sequence
  Synthetic Polynucleotide
gggaaauaagagagaaaagaagaguaagaagaaauauaagagccaccaugggccugcagaccacaaaguggccuucucacggcgcauucuuucugaaguccuggcugaucaucucccugggccuguacagccagguguccaaacugcuggccgccaccgaugauggcgagacaauggugguuacagccagcgccaucgagcagaaccugaaagaugccccugccagcaucagcgugaucacccagcaggaucugcagagaaggcccgugcagaaucugaaggacgugcugaaagaggugcccggcguccagcugacaaacgagggcgauaauagaaagggcguguccaucagaggccuggacagcagcuacacccugauccugaucgacggcaagagagugaacagccggaacgccguguuccggcacaacgacuucgaccugaacuggauccccguggaugccaucgagaggaucgaaguugugcggggaccuaugagcagccuguacggaucugaugcccucggcggcguggucaacaucaucaccaagaagaucggccagaaauggcacggcagcgugaccguggauagcaccauccaagagcacagagacagaggcgacaccuacaacggccaguucuucacaagcggcccucugauugauggcgugcugggcaugaaggccuacggaucucuggccaagagagagaaggacgagcagcagagcagcgccacaacagccacaggcgagacaccuagaaucgagggcuucaccagcagagauggcaacguggaauucgccuggacacccaacgagaaccacgaugugacagccggcuacggcuucgacagacaggacagagauagcgacagccuggacaagaaccggcuggaaagacagaacuacgcccugagccacaacggcagaugggaccugggaaacagcgagcugaaguucuacggcgagaaggucgagaacaagaaccccggcaacagcagcccuaucaccagcgagagcaacagcaucgauggcaaauacgugcugccccuggccuccgugaaucaguuccugacauuuggcggagaguggcggcacgacaagcugucugaugccguuucucugaccggcggcagcagcacaaagacaagcgccucucaguacgcccuguuccuggaagaugaguggcggaucuucgagccccuggcucugacaacaggcaucagaauggacgaccacgagacauacggcgaccacugguccccaagagccuaccugguguacaacgccgccgacacacugaccgugaaaggcggaugggccacagccuuuaaggcuccuagucugcugcagcugagccccgauugggccaccaauucuuguagaggcggcugcagaaucgugggcagcccugaucugaagcccgagacaucugagucuugggagcugggacuguacuacaggggcgaagagggaauccuggaaggcguggaagccagcgugacaaccuucagaaacgacguggacaaccggaucagcaucuccagaacacccgacgugaacgccgcuccuggcuacucuaauuucgugggcuucgagacaaacagcagaggccagagggugcccguguuucgguacuacaacgugaacaaggcccggauccagggcgucgagacagagcugaaggugcccuuuaacgaagccuggaagcugagccugaacuacgcauacaacgacggccgggacguguccaacggcggaaacaaaccucugagcgaccugccuuuccacaccgccaauggcgcccuggauuggaagccugcucagcuggaagauuggagcuucuacguguccggcaacuacgccggcagaaagagagccgauagcgccaccgcuaagacaccaggcggauacgucgugugggauacaggugcugcuuggcaggccaccaagaacgugaaacugagagccggcgugcugaacgugggcgacaaagaccugaagagggacgacuacggcuacaccgaggacggcagacgguacuucauggccguggacuaccgguucugauaauaggcuggagccucgguggccaugcuucuugccccuugggccuccccccagccccuccuccccuuccugcacccguacccccguggucuuugaauaaagucugagugggcggc

SEQ ID NO: 203 - RNA, 841 nt, Artificial Sequence
  Synthetic Polynucleotide
gggaaauaagagagaaaagaagaguaagaagaaauauaagagccaccauggauagcaagggcagcagccagaagggcuccagacugcugcugcuucuggugguguccaaccugcuucugccucaaggcguugugggcacgagccaccacgacagccacggguugcacagggugggaagcggcagcgccaugcagaucuacgagggaaagcugaccgccgagggccugagauuuggaaucguggccagccgguucaaucacgcccugguggauagacugguggaaggcgccaucgaugccauugucagacacggcggcagagaagaggacaucacccucguuagagugcccggcucuugggagauuccuguggcugcuggcgagcuggcccggaaagagaauaucucugccguuaucgccaucggcgugcugaucagaggcgccacaccucacuucgacuauaucgccagcgagguguccaaaggccuggccgaucugagccuggaacugagaaagcccaucaccuucggcgugaucaccgcugacacacuggaacaggccauugagagagccggcaccaagcacggcaacaaaggaugggaagccgcucuguccgccaucgagauggccaaccuguucaagagccugagaggcggccuggugccuagaggaucucaccaccaccaucaccacagcgcuuggagccauccucaguucgagaagugauaauaggcuggagccucgguggccaugcuucuugccccuugggccuccccccagccccuccuccccuuccugcacccguacccccguggucuuugaauaaagucugagugggcggc

SEQ ID NO: 204 - RNA, 2422 nt, Artificial Sequence
  Synthetic Polynucleotide
gggaaauaagagagaaaagaagaguaagaagaaauauaagagccaccauggagacaccugcccagcugcuguuccugcugcugcuguggcugccugacaccaccggggaggagaagaccgacagcgcagcccugaccaacgaggacaccaucguggugaccgccgcucagcagaaccugcaggcgcccggcgugagcaccaucaccgccgacgagaucagaaagaacccuccugccagagacgugagcgagaucaucagaaccaugccuggcgugaaccugaccggcaacagcaccagcggccagagaggcaacaacagacagaucgacaucagaggcaugggcccugagaacacccugauccugaucgacggcaagcccgugaccagcaggaauagcgugagacugggcuggagaggggagcgcgacacaaggggcgauaccgccugggugccuccugagaugaucgagagaaucgaggugcugagaggcccugccgccgccagauacgggaacggagccgccggcggcguggugaacaucaucaccaagaagggcggcagcgaguggcacggcagcuggaacaccuacuucaacgccccugagcacaaggacgagggcgccaccaagagaaccaacuucagccugaacggcccucugggcggcgacuucagcuucagacuguacggcaaccuggacaagacccaggccgacgccagaaacaucaaccagggccaccagagcgagagaaccggcagcuacgccgacacccugccugccggcagagagggcgugaucaacaaggacaucaacggcgugguccgcugggauuucgccccacugcagagccuggagcuggaggccggcuacagcagacaggggaaccuguacgccggcgauacccagaacaccaacaccaaccagcuggugaaggacaacuacggcaaggagacaaauagacuguaccgccagaacuacagccugaccuggaacggcggcugggacaacggggugaccaccagcaacugggugcaguacgagcacaccagaaacagcagaaugcccgaggggcuggccgguggcacugaaggcaucuucgacccuaaagccagccagaaauacgcugacgccgaucugaacgacgugacccugcacagcgaggugagccugccuuucgaccugcucgucaaccagaaccugacccugggcaccgagugggcccagcagagaaugaaggaccagcugagcaacagccagaccuucaugggcggaaacauaccaggcuauagcagcaccaacagaagcccuuacagcaaggccgagaucuucucccuguucgccgagaacaacauggagcugaccgacagcaccaugcugaccccuggcaucagauucgaccaucacagcaucgugggcgacaacuggagccccagccugaaccugagccaaggccugggcgacgacuucacccugaagaugggcaucgccagagccuacaaggccccuagccuguaccagaccaacccuaacuacauccuguacagcaagggccagggcuguuacgcaaccggcgccggcacaggcaucggcugcuacaugaugggcaacgacgaccugaaggccgaaacuagcaucaacaaggagaucggccuggaguucaagagggacggcuggcucgccggagugaccugguuuagaaacgacuacagaaacaagaucgaagccggcaccguaccucuucagcggaucaacaacggcaagaccgacguguaccagugggagaacgugccuaaggccgugguggagggccucgagggcacccucaacgugccugugagcgacaccgugaacuggaccaacaacgugaccuacaugcugcagagcaagaacaaggaaaccggcgagagacugagcaucaucccucaguacacccugaacagcacccugagcuggcaggugagacaggacgugucccugcaguccaccuuuacgugguacggcaagcaggagccuaagaaguacgacuaccagggcaacccugugaccggcaccgacaagcaggccgucagcccauauuccaucgugggacucagcgccaccugggacgugaccaagaacgugagccugaccggagggguugacaaccuguucgacaagagacuguggagagagggcaacgcccagaccgugagggacacucagaccggcgccuacauggccggcgcuggggccuacaccuacaacgagcccggucggaccugguacauguccaucaacacccacuuccaccaucaccaucaccacugauaauaggcuggagccucgguggccaugcuucuugccccuugggccuccccccagccccuccuccccuuccugcacccguacccccguggucuuugaauaaagucugagugggcggc

SEQ ID NO: 205 - RNA, 2404 nt, Artificial Sequence
  Synthetic Polynucleotide
gggaaauaagagagaaaagaagaguaagaagaaauauaagagccaccauggagacaccugcccagcugcuguuccugcugcugcuguggcugccugacaccaccggggaggagaagaccgacagcgcagcccugaccaacgaggacaccaucguggugaccgccgcucagcagaaccugcaggcgcccggcgugagcaccaucaccgccgacgagaucagaaagaacccuccugccagagacgugagcgagaucaucagaaccaugccuggcgugaaccugaccggcaacagcaccagcggccagagaggcaacaacagacagaucgacaucagaggcaugggcccugagaacacccugauccugaucgacggcaagcccgugaccagcaggaauagcgugagacugggcuggagaggggagcgcgacacaaggggcgauaccgccugggugccuccugagaugaucgagagaaucgaggugcugagaggcccugccgccgccagauacgggaacggagccgccggcggcguggugaacaucaucaccaagaagggcggcagcgaguggcacggcagcuggaacaccuacuucaacgccccugagcacaaggacgagggcgccaccaagagaaccaacuucagccugaacggcccucugggcggcgacuucagcuucagacuguacggcaaccuggacaagacccaggccgacgccagaaacaucaaccagggccaccagagcgagagaaccggcagcuacgccgacacccugccugccggcagagagggcgugaucaacaaggacaucaacggcgugguccgcugggauuucgccccacugcagagccuggagcuggaggccggcuacagcagacaggggaaccuguacgccggcgauacccagaacaccaacaccaaccagcuggugaaggacaacuacggcaaggagacaaauagacuguaccgccagaacuacagccugaccuggaacggcggcugggacaacggggugaccaccagcaacugggugcaguacgagcacaccagaaacagcagaaugcccgaggggcuggccgguggcacugaaggcaucuucgacccuaaagccagccagaaauacgcugacgccgaucugaacgacgugacccugcacagcgaggugagccugccuuucgaccugcucgucaaccagaaccugacccugggcaccgagugggcccagcagagaaugaaggaccagcugagcaacagccagaccuucaugggcggaaacauaccaggcuauagcagcaccaacagaagcccuuacagcaaggccgagaucuucucccuguucgccgagaacaacauggagcugaccgacagcaccaugcugaccccuggcaucagauucgaccaucacagcaucgugggcgacaacuggagccccagccugaaccugagccaaggccugggcgacgacuucacccugaagaugggcaucgccagagccuacaaggccccuagccuguaccagaccaacccuaacuacauccuguacagcaagggccagggcuguuacgcaaccggcgccggcacaggcaucggcugcuacaugaugggcaacgacgaccugaaggccgaaacuagcaucaacaaggagaucggccuggaguucaagagggacggcuggcucgccggagugaccugguuuagaaacgacuacagaaacaagaucgaagccggcaccguaccucuucagcggaucaacaacggcaagaccgacguguaccagugggagaacgugccuaaggccgugguggagggccucgagggcacccucaacgugccugugagcgacaccgugaacuggaccaacaacgugaccuacaugcugcagagcaagaacaaggaaaccggcgagagacugagcaucaucccucaguacacccugaacagcacccugagcuggcaggugagacaggacgugucccugcaguccaccuuuacgugguacggcaagcaggagccuaagaaguacgacuaccagggcaacccugugaccggcaccgacaagcaggccgucagcccauauuccaucgugggacucagcgccaccugggacgugaccaagaacgugagccugaccggagggguugacaaccuguucgacaagagacuguggagagagggcaacgcccagaccgugagggacacucagaccggcgccuacauggccggcgcuggggccuacaccuacaacgagcccggucggaccugguacauguccaucaacacccacuucugauaauaggcuggagccucgguggccaugcuucuugccccuugggccuccccccagccccuccuccccuuccugcacccguacccccguggucuuugaauaaagucugagugggcggc

SEQ ID NO: 206 - RNA, 2422 nt, Artificial Sequence
  Synthetic Polynucleotide
gggaaauaagagagaaaagaagaguaagaagaaauauaagagccaccauggagacaccugcccagcugcuguuccugcugcugcuguggcugccugacaccaccggggaggagaagaccgacagcgcagcccugaccaacgaggacaccaucguggugaccgccgcucagcagaaccugcaggcgcccggcgugagcaccaucaccgccgacgagaucagaaagaacccuccugccagagacgugagcgagaucaucagaaccaugccuggcgugaaccuggccggcaacagcgccagcggccagagaggcaacaacagacagaucgacaucagaggcaugggcccugagaacacccugauccugaucgacggcaagcccgugaccagcaggaauagcgugagacugggcuggagaggggagcgcgacacaaggggcgauaccgccugggugccuccugagaugaucgagagaaucgaggugcugagaggcccugccgccgccagauacgggaacggagccgccggcggcguggugaacaucaucaccaagaagggcggcagcgaguggcacggcagcuggaacaccuacuucaacgccccugagcacaaggacgagggcgccaccaagagaaccaacuucgcacugaacggcccucugggcggcgacuucagcuucagacuguacggcaaccuggacaagacccaggccgacgccagaaacaucaaccagggccaccagagcgagagaaccggcagcuacgccgacacccugccugccggcagagagggcgugaucaacaaggacaucaacggcgugguccgcugggauuucgccccacugcagagccuggagcuggaggccggcuacagcagacaggggaaccuguacgccggcgauacccagaacaccaacaccaaccagcuggugaaggacaacuacggcaaggagacaaauagacuguaccgccagaacuacgcacugaccuggaacggcggcugggacaacggggugaccaccagcaacugggugcaguacgagcacaccagaaacagcagaaugcccgaggggcuggccgguggcacugaaggcaucuucgacccuaaagccagccagaaauacgcugacgccgaucugaacgacgugacccugcacagcgaggugagccugccuuucgaccugcucgucaaccagaaccuggcccugggcaccgagugggcccagcagagaaugaaggaccagcugagcaacagccagaccuucaugggcggaaacauaccaggcuauagcagcaccaacagaagcccuuacagcaaggccgagaucuucucccuguucgccgagaacaacauggagcugaccgacagcaccaugcugaccccuggcaucagauucgaccaucacagcaucgugggcgacaacuggagccccagccugaaccugagccaaggccugggcgacgacuucacccugaagaugggcaucgccagagccuacaaggccccuagccuguaccagaccaacccuaacuacauccuguacagcaagggccagggcuguuacgcaaccggcgccggcacaggcaucggcugcuacaugaugggcaacgacgaccugaaggccgaaacuagcaucaacaaggagaucggccuggaguucaagagggacggcuggcucgccggagugaccugguuuagaaacgacuacagaaacaagaucgaagccggcaccguaccucuucagcggaucaacaacggcaagaccgacguguaccagugggagaacgugccuaaggccgugguggagggccucgagggcacccucaacgugccugugagcgacaccgugaacugggccaacaacguggccuacaugcugcagagcaagaacaaggaaaccggcgagagacugagcaucaucccucaguacacccugaacagcgcccugagcuggcaggugagacaggacgugucccugcaguccaccuuuacgugguacggcaagcaggagccuaagaaguacgacuaccagggcaacccugugaccggcaccgacaagcaggccgucagcccauauuccaucgugggacucagcgccaccugggacgugaccaagaacgugagccugaccggagggguugacaaccuguucgacaagagacuguggagagagggcaacgcccagaccgugagggacacucagaccggcgccuacauggccggcgcuggggccuacaccuacaacgagcccggucggaccugguacauguccaucaacacccacuuccaccaucaccaucaccacugauaauaggcuggagccucgguggccaugcuucuugccccuugggccuccccccagccccuccuccccuuccugcacccguacccccguggucuuugaauaaagucugagugggcggc

SEQ ID NO: 207 - RNA, 2404 nt, Artificial Sequence
  Synthetic Polynucleotide
gggaaauaagagagaaaagaagaguaagaagaaauauaagagccaccauggagacaccugcccagcugcuguuccugcugcugcuguggcugccugacaccaccggggaggagaagaccgacagcgcagcccugaccaacgaggacaccaucguggugaccgccgcucagcagaaccugcaggcgcccggcgugagcaccaucaccgccgacgagaucagaaagaacccuccugccagagacgugagcgagaucaucagaaccaugccuggcgugaaccuggccggcaacagcgccagcggccagagaggcaacaacagacagaucgacaucagaggcaugggcccugagaacacccugauccugaucgacggcaagcccgugaccagcaggaauagcgugagacugggcuggagaggggagcgcgacacaaggggcgauaccgccugggugccuccugagaugaucgagagaaucgaggugcugagaggcccugccgccgccagauacgggaacggagccgccggcggcguggugaacaucaucaccaagaagggcggcagcgaguggcacggcagcuggaacaccuacuucaacgccccugagcacaaggacgagggcgccaccaagagaaccaacuucgcacugaacggcccucugggcggcgacuucagcuucagacuguacggcaaccuggacaagacccaggccgacgccagaaacaucaaccagggccaccagagcgagagaaccggcagcuacgccgacacccugccugccggcagagagggcgugaucaacaaggacaucaacggcgugguccgcugggauuucgccccacugcagagccuggagcuggaggccggcuacagcagacaggggaaccuguacgccggcgauacccagaacaccaacaccaaccagcuggugaaggacaacuacggcaaggagacaaauagacuguaccgccagaacuacgcacugaccuggaacggcggcugggacaacggggugaccaccagcaacugggugcaguacgagcacaccagaaacagcagaaugcccgaggggcuggccgguggcacugaaggcaucuucgacccuaaagccagccagaaauacgcugacgccgaucugaacgacgugacccugcacagcgaggugagccugccuuucgaccugcucgucaaccagaaccuggcccugggcaccgagugggcccagcagagaaugaaggaccagcugagcaacagccagaccuucaugggcggaaacauaccaggcuauagcagcaccaacagaagcccuuacagcaaggccgagaucuucucccuguucgccgagaacaacauggagcugaccgacagcaccaugcugaccccuggcaucagauucgaccaucacagcaucgugggcgacaacuggagccccagccugaaccugagccaaggccugggcgacgacuucacccugaagaugggcaucgccagagccuacaaggccccuagccuguaccagaccaacccuaacuacauccuguacagcaagggccagggcuguuacgcaaccggcgccggcacaggcaucggcugcuacaugaugggcaacgacgaccugaaggccgaaacuagcaucaacaaggagaucggccuggaguucaagagggacggcuggcucgccggagugaccugguuuagaaacgacuacagaaacaagaucgaagccggcaccguaccucuucagcggaucaacaacggcaagaccgacguguaccagugggagaacgugccuaaggccgugguggagggccucgagggcacccucaacgugccugugagcgacaccgugaacugggccaacaacguggccuacaugcugcagagcaagaacaaggaaaccggcgagagacugagcaucaucccucaguacacccugaacagcgcccugagcuggcaggugagacaggacgugucccugcaguccaccuuuacgugguacggcaagcaggagccuaagaaguacgacuaccagggcaacccugugaccggcaccgacaagcaggccgucagcccauauuccaucgugggacucagcgccaccugggacgugaccaagaacgugagccugaccggagggguugacaaccuguucgacaagagacuguggagagagggcaacgcccagaccgugagggacacucagaccggcgccuacauggccggcgcuggggccuacaccuacaacgagcccggucggaccugguacauguccaucaacacccacuucugauaauaggcuggagccucgguggccaugcuucuugccccuugggccuccccccagccccuccuccccuuccugcacccguacccccguggucuuugaauaaagucugagugggcggc

SEQ ID NO: 208 - RNA, 967 nt, Artificial Sequence
  Synthetic Polynucleotide
gggaaauaagagagaaaagaagaguaagaagaaauauaagagccaccauggagacuccugcccagcugcuguuccugcugcugcuguggcugccugacaccaccggcaacaucagcgacuacaaggugaugaccuggaaccugcagggaagcuccgccagcaccgagagcaaguggaacgugaacgugagacagcuccugagcggcaccgccggcgucgacauccugauggugcaggaggccggagccgucccuaccagcgccgugccuaccggcagacacauccagccuuucggcgugggcaucccuaucgacgaguacaccuggaaucucggcaccaccagcagacaggacaucagauacaucuaccacagcgccaucgacgugggcgccagaagagugaaccuggccaucgugagcagacagagagccgacaacguguacgugcugaggccuaccaccguggccagcagaccugugaucggcaucggccugggcaacgacguguuccugaccgcccacgcucuggccuccgguggccccgacgcugccgccaucgugagagugaccaucaacuucuucagacagccucagaugagacaccugagcugguuccuggccggcgacuucaacagaagcccugacagacuggagaacgaccugaugaccgagcaccuggagagagugguggccgugcuggccccuaccgagccuacccagaucggcggcggcauccuggacuacggcgugaucguggacagagccccuuacagccagagaguggaggcccugagaaacccucagcuggccagcgaccacuacccuguggccuuccuggccagaagcugcugauaauaggcuggagccucgguggccaugcuucuugccccuugggccuccccccagccccuccuccccuuccugcacccguacccccguggucuuugaauaaagucugagugggcggc

SEQ ID NO: 209 - RNA, 985 nt, Artificial Sequence
  Synthetic Polynucleotide
gggaaauaagagagaaaagaagaguaagaagaaauauaagagccaccauggagacuccugcccagcugcuguuccugcugcugcuguggcugccugacaccaccggcaacaucagcgacuacaaggugaugaccuggaaccugcagggaagcuccgccagcaccgagagcaaguggaacgugaacgugagacagcuccugagcggcaccgccggcgucgacauccugauggugcaggaggccggagccgucccuaccagcgccgugccuaccggcagacacauccagccuuucggcgugggcaucccuaucgacgaguacaccuggaaucucggcaccaccagcagacaggacaucagauacaucuaccacagcgccaucgacgugggcgccagaagagugaaccuggccaucgugagcagacagagagccgacaacguguacgugcugaggccuaccaccguggccagcagaccugugaucggcaucggccugggcaacgacguguuccugaccgcccacgcucuggccuccgguggccccgacgcugccgccaucgugagagugaccaucaacuucuucagacagccucagaugagacaccugagcugguuccuggccggcgacuucaacagaagcccugacagacuggagaacgaccugaugaccgagcaccuggagagagugguggccgugcuggccccuaccgagccuacccagaucggcggcggcauccuggacuacggcgugaucguggacagagccccuuacagccagagaguggaggcccugagaaacccucagcuggccagcgaccacuacccuguggccuuccuggccagaagcugccaucaccaccaccaccacugauaauaggcuggagccucgguggccaugcuucuugccccuugggccuccccccagccccuccuccccuuccugcacccguacccccguggucuuugaauaaagucugagugggcggc

SEQ ID NO: 210 - RNA, 967 nt, Artificial Sequence
  Synthetic Polynucleotide
gggaaauaagagagaaaagaagaguaagaagaaauauaagagccaccauggagacuccugcccagcugcuguuccugcugcugcuguggcugccugacaccaccggcaacaucgcagacuacaaggugaugaccuggaaccugcagggaagcuccgccagcaccgagagcaaguggaacgugaacgugagacagcuccugagcggcaccgccggcgucgacauccugauggugcaggaggccggagccgucccuaccagcgccgugccuaccggcagacacauccagccuuucggcgugggcaucccuaucgacgaguacaccuggaaucucggcaccaccagcagacaggacaucagauacaucuaccacagcgccaucgacgugggcgccagaagagugaaccuggccaucgugagcagacagagagccgacaacguguacgugcugaggccuaccaccguggccagcagaccugugaucggcaucggccugggcaacgacguguuccugaccgcccacgcucuggccuccgguggccccgacgcugccgccaucgugagagugaccaucaacuucuucagacagccucagaugagacaccugagcugguuccuggccggcgacuucaacagaagcccugacagacuggagaacgaccugaugaccgagcaccuggagagagugguggccgugcuggccccuaccgagccuacccagaucggcggcggcauccuggacuacggcgugaucguggacagagccccuuacagccagagaguggaggcccugagaaacccucagcuggccagcgaccacuacccuguggccuuccuggccagaagcugcugauaauaggcuggagccucgguggccaugcuucuugccccuugggccuccccccagccccuccuccccuuccugcacccguacccccguggucuuugaauaaagucugagugggcggc

SEQ ID NO: 211 - RNA, 985 nt, Artificial Sequence
  Synthetic Polynucleotide
gggaaauaagagagaaaagaagaguaagaagaaauauaagagccaccauggagacuccugcccagcugcuguuccugcugcugcuguggcugccugacaccaccggcaacaucgcagacuacaaggugaugaccuggaaccugcagggaagcuccgccagcaccgagagcaaguggaacgugaacgugagacagcuccugagcggcaccgccggcgucgacauccugauggugcaggaggccggagccgucccuaccagcgccgugccuaccggcagacacauccagccuuucggcgugggcaucccuaucgacgaguacaccuggaaucucggcaccaccagcagacaggacaucagauacaucuaccacagcgccaucgacgugggcgccagaagagugaaccuggccaucgugagcagacagagagccgacaacguguacgugcugaggccuaccaccguggccagcagaccugugaucggcaucggccugggcaacgacguguuccugaccgcccacgcucuggccuccgguggccccgacgcugccgccaucgugagagugaccaucaacuucuucagacagccucagaugagacaccugagcugguuccuggccggcgacuucaacagaagcccugacagacuggagaacgaccugaugaccgagcaccuggagagagugguggccgugcuggccccuaccgagccuacccagaucggcggcggcauccuggacuacggcgugaucguggacagagccccuuacagccagagaguggaggcccugagaaacccucagcuggccagcgaccacuacccuguggccuuccuggccagaagcugccaucaccaccaccaccacugauaauaggcuggagccucgguggccaugcuucuugccccuugggccuccccccagccccuccuccccuuccugcacccguacccccguggucuuugaauaaagucugagugggcggc

SEQ ID NO: 212 - RNA, 856 nt, Artificial Sequence
  Synthetic Polynucleotide
gggaaauaagagagaaaagaagaguaagaagaaauauaagagccaccauggagacgccugcccagcugcuguuccugcuccuucuguggcugccugacaccaccggcggcgccuacguggagaacagagaggccuacaaccuggccagcgaccagauggaguucaugcugagagugggcuacaacagcgacaugggcgccggcaucaugcuuaccaacaccuacacccugcagagagacgacgagcugaagcacggcuacaacgagaucgagggcugguacccucuguucaagccuaccgacaagcugaccauccagccuggcggccugaucaacgacaagagcaucggcucuggcggugccguguaccuggacaucaacuacaaguucaccccuugguucaaccugaccgugagaaacagauacaaccacaacaacuacagcagcaccgaccugaacggcgagcuggacaacaacgacagcuacgagauuggcaacuacuggaacuucaucaucaccgauaaguucagcuacaccuucgagccucacuacuucuacaacgugaacgacuucaauaguagcaacggcaccaagcaccacugggagaucaccaacacguucagauacagaaucaacgagcacuggcuuccuuacuucgagcugagguggcuggacagaaacgugggccuguaccacagagagcagaaccagaucagaaucggcgccaaguacuucuucugauaauaggcuggagccucgguggccaugcuucuugccccuugggccuccccccagccccuccuccccuuccugcacccguacccccguggucuuugaauaaagucugagugggcggc

SEQ ID NO: 213 - RNA, 874 nt, Artificial Sequence
  Synthetic Polynucleotide
gggaaauaagagagaaaagaagaguaagaagaaauauaagagccaccauggagacgccugcccagcugcuguuccugcuccuucuguggcugccugacaccaccggcggcgccuacguggagaacagagaggccuacaaccuggccagcgaccagauggaguucaugcugagagugggcuacaacagcgacaugggcgccggcaucaugcuuaccaacaccuacacccugcagagagacgacgagcugaagcacggcuacaacgagaucgagggcugguacccucuguucaagccuaccgacaagcugaccauccagccuggcggccugaucaacgacaagagcaucggcucuggcggugccguguaccuggacaucaacuacaaguucaccccuugguucaaccugaccgugagaaacagauacaaccacaacaacuacagcagcaccgaccugaacggcgagcuggacaacaacgacagcuacgagauuggcaacuacuggaacuucaucaucaccgauaaguucagcuacaccuucgagccucacuacuucuacaacgugaacgacuucaauaguagcaacggcaccaagcaccacugggagaucaccaacacguucagauacagaaucaacgagcacuggcuuccuuacuucgagcugagguggcuggacagaaacgugggccuguaccacagagagcagaaccagaucagaaucggcgccaaguacuucuuccaccaucaccaccaccacugauaauaggcuggagccucgguggccaugcuucuugccccuugggccuccccccagccccuccuccccuuccugcacccguacccccguggucuuugaauaaagucugagugggcggc

SEQ ID NO: 214 - RNA, 856 nt, Artificial Sequence
  Synthetic Polynucleotide
gggaaauaagagagaaaagaagaguaagaagaaauauaagagccaccauggagacgccugcccagcugcuguuccugcuccuucuguggcugccugacaccaccggcggcgccuacguggagaacagagaggccuacaaccuggccagcgaccagauggaguucaugcugagagugggcuacaacagcgacaugggcgccggcaucaugcuuaccaacaccuacacccugcagagagacgacgagcugaagcacggcuacaacgagaucgagggcugguacccucuguucaagccuaccgacaagcugaccauccagccuggcggccugaucaacgacaagagcaucggcucuggcggugccguguaccuggacaucaacuacaaguucaccccuugguucaaccuggccgugagaaacagauacaaccacaacaacuacgcaagcaccgaccugaacggcgagcuggacaacaacgacagcuacgagauuggcaacuacuggaacuucaucaucaccgauaaguucagcuacaccuucgagccucacuacuucuacaacgugaacgacuucaauaguagcaacggcgccaagcaccacugggagaucaccaacacguucagauacagaaucaacgagcacuggcuuccuuacuucgagcugagguggcuggacagaaacgugggccuguaccacagagagcagaaccagaucagaaucggcgccaaguacuucuucugauaauaggcuggagccucgguggccaugcuucuugccccuugggccuccccccagccccuccuccccuuccugcacccguacccccguggucuuugaauaaagucugagugggcggc

SEQ ID NO: 215 - RNA, 874 nt, Artificial Sequence
  Synthetic Polynucleotide
gggaaauaagagagaaaagaagaguaagaagaaauauaagagccaccauggagacgccugcccagcugcuguuccugcuccuucuguggcugccugacaccaccggcggcgccuacguggagaacagagaggccuacaaccuggccagcgaccagauggaguucaugcugagagugggcuacaacagcgacaugggcgccggcaucaugcuuaccaacaccuacacccugcagagagacgacgagcugaagcacggcuacaacgagaucgagggcugguacccucuguucaagccuaccgacaagcugaccauccagccuggcggccugaucaacgacaagagcaucggcucuggcggugccguguaccuggacaucaacuacaaguucaccccuugguucaaccuggccgugagaaacagauacaaccacaacaacuacgcaagcaccgaccugaacggcgagcuggacaacaacgacagcuacgagauuggcaacuacuggaacuucaucaucaccgauaaguucagcuacaccuucgagccucacuacuucuacaacgugaacgacuucaauaguagcaacggcgccaagcaccacugggagaucaccaacacguucagauacagaaucaacgagcacuggcuuccuuacuucgagcugagguggcuggacagaaacgugggccuguaccacagagagcagaaccagaucagaaucggcgccaaguacuucuuccaccaucaccaccaccacugauaauaggcuggagccucgguggccaugcuucuugccccuugggccuccccccagccccuccuccccuuccugcacccguacccccguggucuuugaauaaagucugagugggcggc

SEQ ID NO: 216 - RNA, 898 nt, Artificial Sequence
  Synthetic Polynucleotide
gggaaauaagagagaaaagaagaguaagaagaaauauaagagccaccauggaaaccccugcccagcugcuguuccugcugcugcucuggcugccugacacaaccggcguggacuucguguacagaguggacagcaccccuccugacgugaucuucagagacggcuucagccugcugggcuacaacagaaacuuccagcaguucaucagcggcagaagcugcagcggcgguagcagcgauagcagauacaucgccaccaccagcagcgugaaccagaccuacgccaucgccagagccuacuacagcagaagcaccuucaagggcaaccuguacagauaccagaucagagccgacaacaacuucuacagccugcugccuagcaucaccuaccuggaaacgcagggcggccacuucaacgccuacgagaagaccaugaugagacugcagagagaguacgugagcacccugucaauccugcccgagaacauccagaaggccguggcccugguguacgacagcgccaccggccuggugaaggacggcgugagcaccaugaacgccagcuaccucgggcugagcacaaccagcaacccuggcgugaucccuuuccugccugagccucagaccuacacccagcagagaaucgacgccuucggcccucugaucagcagcugcuucagcaucggcagcgugugccacagccacagaggccagagagccgacguguacaacaugagcuucuacgacgccagaccugugaucgagcugauccuguccaagugauaauaggcuggagccucgguggccaugcuucuugccccuugggccuccccccagccccuccuccccuuccugcacccguacccccguggucuuugaauaaagucugagugggcggc

SEQ ID NO: 217 - RNA, 916 nt, Artificial Sequence
  Synthetic Polynucleotide
gggaaauaagagagaaaagaagaguaagaagaaauauaagagccaccauggaaaccccugcccagcugcuguuccugcugcugcucuggcugccugacacaaccggcguggacuucguguacagaguggacagcaccccuccugacgugaucuucagagacggcuucagccugcugggcuacaacagaaacuuccagcaguucaucagcggcagaagcugcagcggcgguagcagcgauagcagauacaucgccaccaccagcagcgugaaccagaccuacgccaucgccagagccuacuacagcagaagcaccuucaagggcaaccuguacagauaccagaucagagccgacaacaacuucuacagccugcugccuagcaucaccuaccuggaaacgcagggcggccacuucaacgccuacgagaagaccaugaugagacugcagagagaguacgugagcacccugucaauccugcccgagaacauccagaaggccguggcccugguguacgacagcgccaccggccuggugaaggacggcgugagcaccaugaacgccagcuaccucgggcugagcacaaccagcaacccuggcgugaucccuuuccugccugagccucagaccuacacccagcagagaaucgacgccuucggcccucugaucagcagcugcuucagcaucggcagcgugugccacagccacagaggccagagagccgacguguacaacaugagcuucuacgacgccagaccugugaucgagcugauccuguccaagcaccaccaccaucaccacugauaauaggcuggagccucgguggccaugcuucuugccccuugggccuccccccagccccuccuccccuuccugcacccguacccccguggucuuugaauaaagucugagugggcggc

SEQ ID NO: 218 - RNA, 898 nt, Artificial Sequence
  Synthetic Polynucleotide
gggaaauaagagagaaaagaagaguaagaagaaauauaagagccaccauggaaaccccugcccagcugcuguuccugcugcugcucuggcugccugacacaaccggcguggacuucguguacagaguggacagcaccccuccugacgugaucuucagagacggcuucagccugcugggcuacaacagaaacuuccagcaguucaucagcggcagaagcugcagcggcgguagcagcgauagcagauacaucgccaccaccagcagcgugaaccaggccuacgccaucgccagagccuacuacagcagaagcaccuucaagggcaaccuguacagauaccagaucagagccgacaacaacuucuacagccugcugccuagcaucaccuaccuggaaacgcagggcggccacuucaacgccuacgagaagaccaugaugagacugcagagagaguacgugagcacccugucaauccugcccgagaacauccagaaggccguggcccugguguacgacagcgccaccggccuggugaaggacggcgugagcaccaugaacgccagcuaccucgggcugagcacaaccagcaacccuggcgugaucccuuuccugccugagccucagaccuacacccagcagagaaucgacgccuucggcccucugaucagcagcugcuucagcaucggcagcgugugccacagccacagaggccagagagccgacguguacaacaugagcuucuacgacgccagaccugugaucgagcugauccuguccaagugauaauaggcuggagccucgguggccaugcuucuugccccuugggccuccccccagccccuccuccccuuccugcacccguacccccguggucuuugaauaaagucugagugggcggc

SEQ ID NO: 219 - RNA, 916 nt, Artificial Sequence
  Synthetic Polynucleotide
gggaaauaagagagaaaagaagaguaagaagaaauauaagagccaccauggaaaccccugcccagcugcuguuccugcugcugcucuggcugccugacacaaccggcguggacuucguguacagaguggacagcaccccuccugacgugaucuucagagacggcuucagccugcugggcuacaacagaaacuuccagcaguucaucagcggcagaagcugcagcggcgguagcagcgauagcagauacaucgccaccaccagcagcgugaaccaggccuacgccaucgccagagccuacuacagcagaagcaccuucaagggcaaccuguacagauaccagaucagagccgacaacaacuucuacagccugcugccuagcaucaccuaccuggaaacgcagggcggccacuucaacgccuacgagaagaccaugaugagacugcagagagaguacgugagcacccugucaauccugcccgagaacauccagaaggccguggcccugguguacgacagcgccaccggccuggugaaggacggcgugagcaccaugaacgccagcuaccucgggcugagcacaaccagcaacccuggcgugaucccuuuccugccugagccucagaccuacacccagcagagaaucgacgccuucggcccucugaucagcagcugcuucagcaucggcagcgugugccacagccacagaggccagagagccgacguguacaacaugagcuucuacgacgccagaccugugaucgagcugauccuguccaagcaccaccaccaucaccacugauaauaggcuggagccucgguggccaugcuucuugccccuugggccuccccccagccccuccuccccuuccugcacccguacccccguggucuuugaauaaagucugagugggcggc

SEQ ID NO: 220 - RNA, 568 nt, Artificial Sequence
  Synthetic Polynucleotide
gggaaauaagagagaaaagaagaguaagaagaaauauaagagccaccauggagacaccugcccagcugcuguuccugcugcugcuguggcugccugacaccacuggcgaguggaccggcgacaacaccaacgccuacuacagcgacgaggugaucuccgagcugcacgugggacagaucgacaccagcccuuacuucugcaucaagaccgugaaggccaacggcagcggcaccccugugguggccugcgccgugagcaagcagagcaucugggccccuagcuucaaggagcugcuggaccaggccagauacuucuacagcaccggccagagcgugagaauccacgugcagaagaacaucuggaccuacccucuguucgugaacaccuucagcgcgaacgcccuggugggccugagcagcugcagcgccacccagugcuucggccccaagugauaauaggcuggagccucgguggccaugcuucuugccccuugggccuccccccagccccuccuccccuuccugcacccguacccccguggucuuugaauaaagucugagugggcggc

SEQ ID NO: 221 - RNA, 586 nt, Artificial Sequence
  Synthetic Polynucleotide
gggaaauaagagagaaaagaagaguaagaagaaauauaagagccaccauggagacaccugcccagcugcuguuccugcugcugcuguggcugccugacaccacuggcgaguggaccggcgacaacaccaacgccuacuacagcgacgaggugaucuccgagcugcacgugggacagaucgacaccagcccuuacuucugcaucaagaccgugaaggccaacggcagcggcaccccugugguggccugcgccgugagcaagcagagcaucugggccccuagcuucaaggagcugcuggaccaggccagauacuucuacagcaccggccagagcgugagaauccacgugcagaagaacaucuggaccuacccucuguucgugaacaccuucagcgcgaacgcccuggugggccugagcagcugcagcgccacccagugcuucggccccaagcaccaucaccaccaccacugauaauaggcuggagccucgguggccaugcuucuugccccuugggccuccccccagccccuccuccccuuccugcacccguacccccguggucuuugaauaaagucugagugggcggc

SEQ ID NO: 222 - RNA, 568 nt, Artificial Sequence
  Synthetic Polynucleotide
gggaaauaagagagaaaagaagaguaagaagaaauauaagagccaccauggagacaccugcccagcugcuguuccugcugcugcuguggcugccugacaccacuggcgaguggaccggcgacaacaccaacgccuacuacagcgacgaggugaucuccgagcugcacgugggacagaucgacaccagcccuuacuucugcaucaagaccgugaaggccaacggcgcaggcaccccugugguggccugcgccgugagcaagcagagcaucugggccccuagcuucaaggagcugcuggaccaggccagauacuucuacagcaccggccagagcgugagaauccacgugcagaagaacaucuggaccuacccucuguucgugaacaccuucagcgcgaacgcccuggugggccugagcagcugcagcgccacccagugcuucggccccaagugauaauaggcuggagccucgguggccaugcuucuugccccuugggccuccccccagccccuccuccccuuccugcacccguacccccguggucuuugaauaaagucugagugggcggc

SEQ ID NO: 223 - RNA, 586 nt, Artificial Sequence
  Synthetic Polynucleotide
gggaaauaagagagaaaagaagaguaagaagaaauauaagagccaccauggagacaccugcccagcugcuguuccugcugcugcuguggcugccugacaccacuggcgaguggaccggcgacaacaccaacgccuacuacagcgacgaggugaucuccgagcugcacgugggacagaucgacaccagcccuuacuucugcaucaagaccgugaaggccaacggcgcaggcaccccugugguggccugcgccgugagcaagcagagcaucugggccccuagcuucaaggagcugcuggaccaggccagauacuucuacagcaccggccagagcgugagaauccacgugcagaagaacaucuggaccuacccucuguucgugaacaccuucagcgcgaacgcccuggugggccugagcagcugcagcgccacccagugcuucggccccaagcaccaucaccaccaccacugauaauaggcuggagccucgguggccaugcuucuugccccuugggccuccccccagccccuccuccccuuccugcacccguacccccguggucuuugaauaaagucugagugggcggc

SEQ ID NO: 224 - RNA, 1468 nt, Artificial Sequence
  Synthetic Polynucleotide
gggaaauaagagagaaaagaagaguaagaagaaauauaagagccaccauggaaaccccugcccagcugcuguuccugcugcugcuguggcugccugacaccaccggcaugaacacccaggcccuguucugccugagccugccucucgucaucgccgguugcgcccagcagaccagcacccagaccgccagagacagcgccuucgcccagagccagcagccuagcguguucagcgacaucuaccagagaagcccugaggugaccagaagcggcagauacacccuggugagcaucaagagcgccgacgcccagagagagccucugaaccagcugaucgacaucaccaugccuggccagcuggugaacagcgugggcgacggcuucagauaccugcuguuccagagcggcuacagccugugcggccacuacggagccgacuucagcgagcugcugaacagaccucugccugccgugcagagaaagaucggcccuaugagacugagcgaagcccugcagguggucgcgggcccugccuggagaaugagcguggacgaggugaacagagaggugugcuucgugcugagagacgccuacagagugcaggccaagaccaaggcccugaccagcgugacuccagccguuaccggcgccgcccggacauccuccccugagacuaccagaaacccuuacaccggcgugcugccuggcaacaaggccgagcccgugaucgccggccaguaccuggccgugaagaagggcaccgagcugaagccugugagccagagccugccugugccugcccugaggccuggcggcaccccugugaccucgggcguguccucugccccuccugugaagcaggugagcagccccgccccuagaaacccuuucaccggcaagaaccugaccggcaccacccugagcgccagccacagucccgcuccggugggcgagaaggcccaggcaaagacgccuagcccuagcaccgugaaggccgugaacccugccaccgccaccgugcucgccaccaccacuaagccucugaccggaacucccguggccgccguagccagcggaccugaguggaaagcugucgugggcucgacccugaaggagagccugaccgacugggccggcagagccgauugccccggcggcgggcacuggguggugaucuggcagaccccuaccgacuacagaaucgacgccccucugguguucaagggcaacuucgagacagcccuggugcagguguucgaccuguacaagaaggccgacaagccccuguucgccgaggccagcagacugcagugccugguguccguggccgacaaacccgccgacagaagcugauaauaggcuggagccucgguggccuagcuucuugccccuugggccuccccccagccccuccuccccuuccugcacccguacccccguggucuuugaauaaagucugagugggcggc

SEQ ID NO: 225 - RNA, 1468 nt, Artificial Sequence
  Synthetic Polynucleotide
gggaaauaagagagaaaagaagaguaagaagaaauauaagagccaccauggaaaccccugcccagcugcuguuccugcugcugcuguggcugccugacaccaccggcaugaacacccaggcccuguucugccugagccugccucucgucaucgccgguugcgcccagcagaccagcacccagaccgccagagacagcgccuucgcccagagccagcagccuagcguguucagcgacaucuaccagagaagcccugaggugaccagaagcggcagauacacccuggugagcaucaagagcgccgacgcccagagagagccucugaaccagcugaucgacaucaccaugccuggccagcuggugaacagcgugggcgacggcuucagauaccugcuguuccagagcggcuacagccugugcggccacuacggagccgacuucagcgagcugcugaacagaccucugccugccgugcagagaaagaucggcccuaugagacugagcgaagcccugcagguggucgcgggcccugccuggagaaugagcguggacgaggugaacagagaggugugcuucgugcugagagacgccuacagagugcaggccaagaccaaggcccugaccagcgugacuccagccguuaccggcgccgcccggacauccuccccugagacuaccagaaacccuuacaccggcgugcugccuggcaacaaggccgagcccgugaucgccggccaguaccuggccgugaagaagggcaccgagcugaagccugugagccagagccugccugugccugcccugaggccuggcggcaccccugugaccucgggcguguccucugccccuccugugaagcaggugagcagccccgccccuagaaacccuuucaccggcaagaaccuggccggcaccacccugagcgccagccacagucccgcuccggugggcgagaaggcccaggcaaagacgccuagcccuagcaccgugaaggccgugaacccugccaccgccaccgugcucgccaccaccacuaagccucugaccggaacucccguggccgccguagccagcggaccugaguggaaagcugucgugggcucgacccugaaggagagccugaccgacugggccggcagagccgauugccccggcggcgggcacuggguggugaucuggcagaccccuaccgacuacagaaucgacgccccucugguguucaagggcaacuucgagacagcccuggugcagguguucgaccuguacaagaaggccgacaagccccuguucgccgaggccagcagacugcagugccugguguccguggccgacaaacccgccgacagaagcugauaauaggcuggagccucgguggccuagcuucuugccccuugggccuccccccagccccuccuccccuuccugcacccguacccccguggucuuugaauaaagucugagugggcggc

SEQ ID NO: 226 - RNA, 1618 nt, Artificial Sequence
  Synthetic Polynucleotide
gggaaauaagagagaaaagaagaguaagaagaaauauaagagccaccauggagacuccugcccagcugcuguuccugcugcugcuguggcugccugacaccaccggccagaccaccaacagaagcaccgccaaggacgagauccugaccggcagcuacaucgacccuaccaagaccagauuuccccuggcugauuauagccagagcguggacaaguggaucccuccugacucagccgacuacaccaucccugugaucgacagcgccacccagcagagauacuucagcgcccugaagucccacuacuucggcauggacagcgaggcccacagcccuuggaacgacuucuacaucaccgcccugcugaagaagaacgccgcccaggccagagacgccagcaucaagcaguuucuguccgacggcagcgccuacuggggcgagaacuucagacuguacaccagccgguggaaggaggaggugagaggcaacaccgacacccagaucgacaacaucuaccacgccagccgaaggggcaucauggugagagagagccuggugagagcccugccuaccgacgacccucuguucaacgacccuagacaggccggcgagggcuacccuuucgacaaccugcagaugagcagccugcgcccuggcaccccuguguacacccugaccaagagcaaggaccagcgguggcaguacgugguguccccugccgugacagggugggugcacagcgaggacaucgccagcaccgaccagaaguucaucacccagugggugcugcuggcccauaagcagcucggcgccuucaucaacgccccuguuuccgugcacgccgccggcguguacuacuucaccggcagacccggaaccauccugccuuucagacaccagagagccgggcaguuccugauagccgccccgguacgcgggagcaacggccgggccuuuauccacuggguguggcugagcggcaacgaguucaccgccaugccuuggaagaugaccccugagaacaucgccguccugaugaaggccaugcacggcgccccuuacggcuggggcaacuucaacuucuacaacgacugcagcgccgaggugagaagccugcugaugccuuucggcaucuuccugccccgucacuccagcgcccaggucgagagcgccggcagagugguggaccugagccacaagaacccucagaugagaaucgacuaccugaccagauacggcaaggccuucaccacacucguguacauccccggccacaucaugcuguacaucggcaacacagccaugaacggccagguggugccuaugaccuaccagaacaucuggggccucagaccuaaccacgccaacagcagaagcaucaucggcgaggccguguuccugccucugcugagauucuacccugagaacccugagcugaucagccuggccggcaaggugcuguucaagcugggcuacaucgagcaccaucaccaccaccacugauaauaggcuggagccucgguggccuagcuucuugccccuugggccuccccccagccccuccuccccuuccugcacccguacccccguggucuuugaauaaagucugagugggcggc

SEQ ID NO: 227 - RNA, 1519 nt, Artificial Sequence
  Synthetic Polynucleotide
gggaaauaagagagaaaagaagaguaagaagaaauauaagagccaccauggagacuccugcccagcugcuguuccugcugcugcuguggcugccugacaccaccggcguggacaaguggaucccuccugacucagccgacuacaccaucccugugaucgacagcgccacccagcagagauacuucagcgcccugaagucccacuacuucggcauggacagcgaggcccacagcccuuggaacgacuucuacaucaccgcccugcugaagaagaacgccgcccaggccagagacgccagcaucaagcaguuucuguccgacggcagcgccuacuggggcgagaacuucagacuguacaccagccgguggaaggaggaggugagaggcaacaccgacacccagaucgacaacaucuaccacgccagccgaaggggcaucauggugagagagagccuggugagagcccugccuaccgacgacccucuguucaacgacccuagacaggccggcgagggcuacccuuucgacaaccugcagaugagcagccugcgcccuggcaccccuguguacacccugaccaagagcaaggaccagcgguggcaguacgugguguccccugccgugacagggugggugcacagcgaggacaucgccagcaccgaccagaaguucaucacccagugggugcugcuggcccauaagcagcucggcgccuucaucaacgccccuguuuccgugcacgccgccggcguguacuacuucaccggcagacccggaaccauccugccuuucagacaccagagagccgggcaguuccugauagccgccccgguacgcgggagcaacggccgggccuuuauccacuggguguggcugagcggcaacgaguucaccgccaugccuuggaagaugaccccugagaacaucgccguccugaugaaggccaugcacggcgccccuuacggcuggggcaacuucaacuucuacaacgacugcagcgccgaggugagaagccugcugaugccuuucggcaucuuccugccccgucacuccagcgcccaggucgagagcgccggcagagugguggaccugagccacaagaacccucagaugagaaucgacuaccugaccagauacggcaaggccuucaccacacucguguacauccccggccacaucaugcuguacaucggcaacacagccaugaacggccagguggugccuaugaccuaccagaacaucuggggccucagaccuaaccacgccaacagcagaagcaucaucggcgaggccguguuccugccucugcugagauucuacccugagaacccugagcugaucagccuggccggcaaggugcuguucaagcugggcuacaucgagcaccaucaccaccaccacugauaauaggcuggagccucgguggccuagcuucuugccccuugggccuccccccagccccuccuccccuuccugcacccguacccccguggucuuugaauaaagucugagugggcggc

SEQ ID NO: 228 - RNA, 646 nt, Artificial Sequence
  Synthetic Polynucleotide
gggaaauaagagagaaaagaagaguaagaagaaauauaagagccaccauggaaacgccugcccagcugcuguuccugcugcugcuguggcugccugacaccaccggcgacagccugagcggcgacguguacaccgccagcgaggccaagcaggugcagaacguggccuacggcaccaucgugaacgucagaccugugcagauccagggcggggacgacagcaacgugaucggcgccauuggaggugccgugcugggcggcuuccugggcaacacuaucggcggcggcaccggcagaagccuggccacagccgccggagccguggcuggcggcguggccggucagggagugcagagcgccaugaacaaggcccagggcguggagcuggagaucagaaaggacgacggcaacaccaucaugguggugcagaagcaagggaacaccagauucagcgccggccagagaguggugcuggccagcaacggcgcccaggugaccgugagcccucgccaucaccaccaccaccacugauaauaggcuggagccucgguggccuagcuucuugccccuugggccuccccccagccccuccuccccuuccugcacccguacccccguggucuuugaauaaagucugagugggcggc

SEQ ID NO: 229 - RNA, 971 nt, Artificial Sequence
  Synthetic Polynucleotide
gggaaauaagagagaaaagaagaguaagaagaaauauaagaccccggcgccgccaccauggagacuccugcccagcugcuguuccugcugcugcuguggcugccugacaccaccggcuacaaggugaugaccuggaaccugcagggaagcuccgccagcaccgagagcaaguggaacgugaacgugagacagcuccugagcggcaccgccggcgucgacauccugauggugcaggaggccggagccgucccuaccagcgccgugccuaccggcagacacauccagccuuucggcgugggcaucccuaucgacgaguacaccuggaaucucggcaccaccagcagacaggacaucagauacaucuaccacagcgccaucgacgugggcgccagaagagugaaccuggccaucgugagcagacagagagccgacaacguguacgugcugaggccuaccaccguggccagcagaccugugaucggcaucggccugggcaacgacguguuccugaccgcccacgcucuggccuccgguggccccgacgcugccgccaucgugagagugaccaucaacuucuucagacagccucagaugagacaccugagcugguuccuggccggcgacuucaacagaagcccugacagacuggagaacgaccugaugaccgagcaccuggagagagugguggccgugcuggccccuaccgagccuacccagaucggcggcggcauccuggacuacggcgugaucguggacagagccccuuacagccagagaguggaggcccugagaaacccucagcuggccagcgaccacuacccuguggccuuccugcaucaccaccaccaccacugauaauaggcuggagccucgguggccuagcuucuugccccuugggccuccccccagccccuccuccccuuccugcacccguacccccguggucuuugaauaaagucugagugggcggc

SEQ ID NO: 230 - RNA, 971 nt, Artificial Sequence
  Synthetic Polynucleotide
gggaaauaagagagaaaagaagaguaagaagaaauauaagaccccggcgccgccaccauggagacuccugcccagcugcuguuccugcugcugcuguggcugccugacaccaccggcuacaaggugaugaccuggaaccugcagggaagcuccgccagcaccgagagcaaguggaacgugaacgugagacagcuccugagcggcaccgccggcgucgacauccugauggugcaggaggccggagccgucccuaccagcgccgugccuaccggcagacacauccagccuuucggcgugggcaucccuaucgacgaguacaccuggaaucucggcaccaccagcagacaggacaucagauacaucuaccacagcgccaucgacgugggcgccagaagagugaaccuggccaucgugagcagacagagagccgacaacguguacgugcugaggccuaccaccguggccagcagaccugugaucggcaucggccugggcaacgacguguuccugaccgcccaggcucuggccuccgguggccccgacgcugccgccaucgugagagugaccaucaacuucuucagacagccucagaugagacaccugagcugguuccuggccggcgacuucaacagaagcccugacagacuggagaacgaccugaugaccgagcaccuggagagagugguggccgugcuggccccuaccgagccuacccagaucggcggcggcauccuggacuacggcgugaucguggacagagccccuuacagccagagaguggaggcccugagaaacccucagcuggccagcgaccacuacccuguggccuuccugcaucaccaccaccaccacugauaauaggcuggagccucgguggccuagcuucuugccccuugggccuccccccagccccuccuccccuuccugcacccguacccccguggucuuugaauaaagucugagugggcggc

SEQ ID NO: 231 - RNA, 962 nt, Artificial Sequence
  Synthetic Polynucleotide
gggaaauaagagagaaaagaagaguaagaagaaauauaagaccccggcgccgccaccauggagacaccugcccagcugcuguuccugcugcugcuguggcugcccgacaccaccggccaggccccuaucagcagcgugggcagcggaagcguggaggacagagugacccagcuggagagaaucagcaacgcccacagccagcugcucacccagcuccagcagcagcugagcgacaaccagagcgacaucgacagccugagaggccagauccaggagaaccaguaccagcugaaccaggugauggagagacagaagcagaucaugcugcagcugggaagccugaacaacggcggagccgcucagccugcagccggcgaccagaguggcgccgcaacagccgccacaccagccccugacgcggguacagccaccagcggugcucccgugcagagcggcgacgccaacaccgacuacaacgccgccaucgcccuggugcaggacaagagcagacaggacgacgcuaucguggccuuccagaacuucaucaagaaguaccccgacagcaccuaccagcccaacgccaacuacuggcugggccagcugaacuacaacaagggcaagaaggacgacgccgccuacuacuucgccagcguggugaagaacuaccccaagagccccaaagcggccgacgccauguacaaggugggcgugaucaugcaggacaagggcgacaccgccaaggccaaggccguguaccagcaggugaucaacaaguaccccgguacugacggcgccaagcaggcccagaagagacugaacgccaugcaccaucaccaucaccacugauaauaggcuggagccucgguggccuagcuucuugccccuugggccuccccccagccccuccuccccuuccugcacccguacccccguggucuuugaauaaagucugagugggcggc

SEQ ID NO: 232 - RNA, 962 nt, Artificial Sequence
  Synthetic Polynucleotide
gggaaauaagagagaaaagaagaguaagaagaaauauaagaccccggcgccgccaccauggagacaccugcccagcugcuguuccugcugcugcuguggcugcccgacaccaccggccaggccccuaucagcagcgugggcagcggaagcguggaggacagagugacccagcuggagagaaucagcaacgcccacagccagcugcucacccagcuccagcagcagcugagcgacaaccaggccgacaucgacagccugagaggccagauccaggagaaccaguaccagcugaaccaggugauggagagacagaagcagaucaugcugcagcugggaagccugaacaacggcggagccgcucagccugcagccggcgaccagaguggcgccgcaacagccgccacaccagccccugacgcggguacagccaccagcggugcucccgugcagagcggcgacgccaacaccgacuacaacgccgccaucgcccuggugcaggacaagagcagacaggacgacgcuaucguggccuuccagaacuucaucaagaaguaccccgacagcaccuaccagcccaacgccaacuacuggcugggccagcugaacuacaacaagggcaagaaggacgacgccgccuacuacuucgccagcguggugaagaacuaccccaagagccccaaagcggccgacgccauguacaaggugggcgugaucaugcaggacaagggcgacaccgccaaggccaaggccguguaccagcaggugaucaacaaguaccccgguacugacggcgccaagcaggcccagaagagacugaacgccaugcaccaucaccaucaccacugauaauaggcuggagccucgguggccuagcuucuugccccuugggccuccccccagccccuccuccccuuccugcacccguacccccguggucuuugaauaaagucugagugggcggc

SEQ ID NO: 233 - RNA, 755 nt, Artificial Sequence
  Synthetic Polynucleotide
gggaaauaagagagaaaagaagaguaagaagaaauauaagaccccggcgccgccaccauggaaaccccagcccagcuccuguuccugcugcugcuguggcuucccgacaccaccggcgagaacaagagcuacuaccagcugcccaucgcccagagcggcgugcagagcacagccagccagggcaacagacugcuguggguggagcaggugagcgugcccgacuaccuggccggcaacggcgugguguaccagaccagcgacgugcaguacgugaucgccaacaacaaccugugggccagcccgcuggaccagcagcugagaaacacccugguggccaaccugagcgccagacuccccggcugggucguugccucccagccccugggcaccacccaggacacccugaacgugaccgugaccggcuuccacggcagauacgacggcaaggugaucgugagcggcgaguggcugcugaaccacaacggccagcugaucaagaggcccuuccacaucgaggccagccagcagaaggacggcuacgacgagauggugaaggugcuggccagcgccuggagccaggaggccgcugccauagccgacgagaucaagagacugccccaccaucaccaccaccacugauaauaggcuggagccucgguggccuagcuucuugccccuugggccuccccccagccccuccuccccuuccugcacccguacccccguggucuuugaauaaagucugagugggcggc

SEQ ID NO: 234 - RNA, 755 nt, Artificial Sequence
  Synthetic Polynucleotide
gggaaauaagagagaaaagaagaguaagaagaaauauaagaccccggcgccgccaccauggaaaccccagcccagcuccuguuccugcugcugcuguggcuucccgacaccaccggcgagaacaaggccuacuaccagcugcccaucgcccagagcggcgugcagagcacagccagccagggcaacagacugcuguggguggagcaggugagcgugcccgacuaccuggccggcaacggcgugguguaccagaccagcgacgugcaguacgugaucgccaacaacaaccugugggccagcccgcuggaccagcagcugagaaacacccugguggccaaccuggccgccagacuccccggcugggucguugccucccagccccugggcaccacccaggacacccugaacguggccgugaccggcuuccacggcagauacgacggcaaggugaucgugagcggcgaguggcugcugaaccacaacggccagcugaucaagaggcccuuccacaucgaggccagccagcagaaggacggcuacgacgagauggugaaggugcuggccagcgccuggagccaggaggccgcugccauagccgacgagaucaagagacugccccaccaucaccaccaccacugauaauaggcuggagccucgguggccuagcuucuugccccuugggccuccccccagccccuccuccccuuccugcacccguacccccguggucuuugaauaaagucugagugggcggc

SEQ ID NO: 235 - RNA, 60 nt, Artificial Sequence
  Synthetic Polynucleotide
auggaaaccccugcucagcugcuguuccugcugcugcuguggcugccugauacaacaggc

SEQ ID NO: 236 - RNA, 11 nt, Artificial Sequence
  Synthetic Polynucleotide
gggauccuacc

(19) United States
(12) Patent Application Publication
(10) Pub. No.: US 2019/0192646 A1
(43) Pub. Date: Jun. 27, 2019

(54) SALMONELLA VACCINES

(71) Applicant: ModernaTX, Inc., Cambridge, MA (US)

(72) Inventors: Nadia Cohen, Cambridge, MA (US); Marie-Angele Andjoua Messou, Boston, MA (US); Samantha Calabrese, Newton, NH (US); Elisabeth Narayanan, Cambridge, MA (US)

(73) Assignee: ModernaTX, Inc., Cambridge, MA (US)

(21) Appl. No.: 16/180,076

(22) Filed: Nov. 5, 2018

Related U.S. Application Data
(60) Provisional application No. 62/589,192, filed on Nov. 21, 2017.
(60) Provisional application No. 62/581,562, filed on Nov. 3, 2017.

(51) Int. Cl. A61K 39/112; A61P 31/04; C07K 14/255; A61K 9/51; A61K 9/00

(52) U.S. Cl. CPC A61K 39/0275; A61P 31/04; C07K 14/255; A61K 2039/70; A61K 9/5146; A61K 9/0019; A61K 9/5123

(57) ABSTRACT

The disclosure relates to Salmonella ribonucleic acid vaccines as well as methods of using the vaccines and compositions comprising the vaccines.

### RELATED APPLICATIONS

This application claims the benefit under 35 U.S.C. § 119(e) of U.S. provisional application No. 62/589,192, filed Nov. 21, 2017, and U.S. provisional application No. 62/581,562, filed Nov. 3, 2017. Each of which is incorporated by reference herein in its entirety.

### BACKGROUND

Salmonella infection (salmonellosis) causes approximately one million foodborne illnesses yearly in the United States, with 19,000 hospitalizations and 380 deaths. Most individuals infected with Salmonella develop diarrhea, fever, and abdominal cramps 12 to 72 hours after infection. The illness usually lasts 4 to 7 days, and most recover without treatment. In some, however, the symptoms are so severe that the patient needs to be hospitalized. In these patients, the Salmonella infection may spread from the intestines to the blood stream, and then to other body sites, which can result in death unless the person is treated promptly with antibiotics. The elderly, infants, and those with impaired immune systems are more likely to have a severe infection. Currently, there is no vaccine to prevent salmonellosis.

### SUMMARY

Provided herein, in some embodiments, are RNA (e.g., mRNA) vaccines, such as multivalent RNA vaccines, that elicit potent neutralizing antibodies and robust T cell responses against Salmonella antigens. The RNA vaccines of the present disclosure, despite encoding bacterial antigens, are highly stable and highly expressed on the surface of mammalian cells. Thus, some aspects of the present disclosure provide Salmonella vaccines comprising a RNA having an open reading frame (ORF) encoding a (at least one) Salmonella antigen, wherein intramuscular (IM) administration of a therapeutically effective amount of the vaccine to a subject induces in the subject a neutralizing antibody titer and/or a T cell immune response (e.g., a CD4⁺ and/or a CD8⁺ T cell response).

In some aspects, the present disclosure provides a multivalent Salmonella vaccine, comprising (a) a RNA having an ORF encoding two (at least two) Salmonella antigens, or (b) two RNAs (at least two RNAs), each having an ORF encoding a Salmonella antigen, wherein IM administration of a therapeutically effective amount of the vaccine to a subject induces in the subject a neutralizing antibody titer and/or a T cell immune response.

In some embodiments, the vaccine comprises a (at least one) RNA having an ORF encoding two (at least two) Salmonella antigens formulated in a lipid nanoparticle. In some embodiments, the vaccine comprises two (at least two) RNAs, each having an ORF encoding a (at least one) Salmonella antigen, wherein the two RNAs are formulated together in a single lipid nanoparticle. In some embodiments, the vaccine comprises two (at least two) RNAs, each having an ORF encoding a (at least one) Salmonella antigen, wherein each RNA is formulated separately in a single lipid nanoparticle (one RNA in one lipid nanoparticle, the other RNA in another lipid nanoparticle). In some embodiments, the vaccine further comprises at least one (e.g., 1, 2, 3, 4, 5, 6, 7, 8, 9 or 10) additional RNA having an ORF encoding at least one (e.g., 1, 2, 3, 4, 5, 6, 7, 8, 9 or 10) additional Salmonella antigen. The additional RNA(s) may be formulated with one of the other RNA(s) or may be formulated separately.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, the Salmonella antigens are selected from the group consisting of: SseB, Mig14, OmpL, OmpC, OmpD, OmpF, IroN, CirA, FepA, T0937, FliC, PilL, PltB, PltA, CdtB, SlyB, STY1086 and STY0796. For example, the Salmonella antigens may include PltB, PltA, CdtB. In some embodiments, the Salmonella antigens include PltB, PltA, CdtB and at least one Salmonella antigen selected from the group consisting of: SseB, Mig14, OmpL, OmpC, OmpD, OmpF, IroN, CirA, FepA, T0937, FliC, PilL, SlyB, STY1086 and STY0796.

In some embodiments, each Salmonella antigen is of a different serotype. For example, the serotypes may be selected from the group consisting of: enterica (serotype I), salamae (serotype II), arizonae (Ma), diarizonae (Mb), houtenae (IV), and indica (VI).

In some embodiments, the Salmonella antigens are fused to a scaffold moiety. For example, the Salmonella antigens may be fused to a scaffold moiety is selected from the group consisting of: ferritin, encapsulin, lumazine synthase, hepatitis B surface antigen, and hepatitis B core antigen.

In some embodiments, the neutralizing antibody titer is at least 100 neutralizing units per milliliter (U/ml) (e.g., 150, 200, 250, 300, 350, 400, or 450 U/ml). In some embodiments, the neutralizing antibody titer is at least 500 U/ml (e.g., 550, 600, 650, 700, 750, 800, 850, 900, or 950 U/ml) or at least 1000 U/ml (e.g., 1500, 2000, 2500, 3000, 3500, 4000, 4500, 5000, 5500, 6000, 6500, 7000, 7500, 8000, 8500, 9000, or 9500 U/ml). In some embodiments, the neutralizing antibody titer is at least 10,000 U/ml.

In some embodiments, the neutralizing antibody titer is induced in the subject following fewer than three (one or two) doses of the vaccine.

In some embodiments, the Salmonella antigen is expressed on the surface of cells of the subject (e.g., a human subject). In some embodiments, the subject is immunocompromised (e.g., has an autoimmune condition and/or is an elderly subject).

In some embodiments, the neutralizing antibody titer is induced within 20 days (e.g., within 10 or 15 days) following a single 10-100 μg dose of the vaccine. In some embodiments, the neutralizing antibody titer is induced within 20 days following a 10 μg dose of the vaccine. In some embodiments, the neutralizing antibody titer is induced within 20 days following a 20 μg dose of the vaccine. In some embodiments, the neutralizing antibody titer is induced within 20 days following a 30 μg dose of the vaccine. In some embodiments, the neutralizing antibody titer is induced within 20 days following a 40 μg dose of the vaccine. In some embodiments, the neutralizing antibody titer is induced within 20 days following a 50 μg dose of the vaccine. In some embodiments, the neutralizing antibody titer is induced within 20 days following a 60 μg dose of the vaccine. In some embodiments, the neutralizing antibody titer is induced within 20 days following a 70 μg dose of the vaccine. In some embodiments, the neutralizing antibody titer is induced within 20 days following a 80 μg dose of the vaccine. In some embodiments, the neutralizing antibody titer is induced within 20 days following a 90 μg dose of the vaccine. In some embodiments, the neutralizing antibody titer is induced within 20 days following a 100 μg dose of the vaccine.

In some embodiments, the neutralizing antibody titer is induced within 40 days following a second 10-100 μg dose (e.g., 10 μg, 20 μg, 30 μg, 40 μg, 50 μg, 60 μg, 70 μg, 80 μg, 90 μg, or 100 μg dose) of the vaccine.

In some embodiments, the T cell immune response comprises a CD4⁺ T cell immune response. In some embodiments, the T cell immune response comprises a CD8⁺ T cell immune response. In some embodiments, the T cell immune response comprises a CD4⁺ T cell immune response and a CD8⁺ T cell immune response.

In some embodiments, the RNA comprises or consists of messenger RNA (mRNA).

In some embodiments, the RNA further comprises a 5′ UTR (e.g., SEQ ID NO: 3 or 140) and/or a 3′ UTR (e.g., SEQ ID NO: 4 or 129).

In some embodiments, the Salmonella antigen is fused to a signal peptide.

In some embodiments, the RNA is unmodified. In other embodiments, the RNA comprise at least one modified nucleotide. For example, at least 80% of the uracil in the ORF may comprise a 1-methyl-pseudouridine modification.

Other aspects of the present disclosure provide a method comprising administering to a subject the Salmonella vaccine as provided herein in a therapeutically effective amount to induce in the subject a neutralizing antibody titer and/or a T cell immune response.

In some embodiments, the efficacy of the Salmonella vaccine is at least 80% (e.g., 80%, 85%, 90% or 95%) relative to unvaccinated control subjects (e.g. human subjects). In some embodiments, detectable levels of Salmonella antigen are produced in the serum of the subject at 1-72 hours post administration of the vaccine.

In some embodiments, a neutralizing antibody titer of at least 100 U/ml (e.g., 150, 200, 250, 300, 350, 400, or 450 U/ml) is produced in the serum of the subject at 1-72 hours post administration of the vaccine. For example, a neutralizing antibody titer of at least 500 U/ml (e.g., 550, 600, 650, 700, 750, 800, 850, 900, or 950 U/ml) or at least 1000 U/ml (e.g., 1500, 2000, 2500, 3000, 3500, 4000, 4500, 5000, 5500, 6000, 6500, 7000, 7500, 8000, 8500, 9000, or 9500 U/ml) may be produced in the serum of the subject at 1-72 hours post administration of the vaccine.

In some embodiments, the therapeutically effective amount is a total dose of 20 μg-200 μg. For example, the therapeutically effective amount may be a total dose of 50 μg-100 μg.

The LNP used in the studies described herein has been used previously to deliver siRNA in various animal models as well as in humans. In view of the observations made in association with the siRNA delivery of LNP formulations, the fact that LNP is useful in vaccines is quite surprising. It has been observed that therapeutic delivery of siRNA formulated in LNP causes an undesirable inflammatory response associated with a transient IgM response, typically leading to a reduction in antigen production and a compromised immune response. In contrast to the findings observed with siRNA, the LNP-mRNA formulations of the invention are demonstrated herein to generate enhanced IgG levels, sufficient for prophylactic and therapeutic methods rather than transient IgM responses.

### BRIEF DESCRIPTION OF THE DRAWINGS

FIGS. 1A-1B show CD4⁺ (FIG. 1A) and CD8⁺ (FIG. 1B) T cell responses in mice following immunization on days 1 and 29 with a vaccine comprising mRNA encoding Salmonella SseB antigen encapsulated in lipid nanoparticles. Two doses, 2 μg and 10 μg, were tested. Levels of IFNγ, TNFα, and IL2 were measured.

FIGS. 2A-2B show CD4⁺ (FIG. 2A) and CD8⁺ (FIG. 2B) T cell responses in mice immunization on days 1 and 29 with a vaccine comprising mRNA encoding Salmonella Mig14 antigen or a non-glycosylated (NGM) version of Salmonella Mig14 antigen encapsulated in lipid nanoparticles. Two doses, 2 μg and 10 μg, were tested. Levels of IFNγ, TNFα, and IL2 were measured.

### DETAILED DESCRIPTION

Salmonella is a genus of intracellular rod-shaped gram-negative bacterial pathogens of the Enterobacteriaceae family. There are two species of Salmonella: Salmonella bongori and Salmonella enterica. Salmonella enterica, found worldwide in all warm-blood animals and in the environment, is further divided into six subspecies that that include over 2,500 serotypes, many of which (e.g., nontyphoidal serotypes) cause illness. These six subspecies are enterica (serotype I), salamae (serotype II), arizonae (Ma), diarizonae (111b), houtenae (IV), and indica (VI).

Infection with nontyphoidal serotypes of Salmonella generally results in food poisoning, while infection with typhoidal serotypes, such as Salmonella Typhi, Paratyphi A, Paratyphi B and Paratyphi C, causes typhoid fever. Salmonella serotypes that cause typhoid fever are strictly adapted to humans or higher primates. These salmonellae can pass through the lymphatic system of the intestine into the blood of patients, invading various organs (e.g., liver, spleen, and kidneys) to form secondary foci. Endotoxins first act on the vascular and nervous apparatus, resulting in increased permeability and decreased tone of the vessels, upset of thermal regulation, vomiting and diarrhea. In severe forms of the disease, enough liquid and electrolytes are lost to upset water-salt metabolism, decrease circulating blood volume and arterial pressure, and cause hypovolemic shock. Septic shock may also develop. Shock of mixed character (with signs of both hypovolemic and septic shock) is more common in severe salmonellosis. Oliguria and azotemia may develop in severe cases as a result of renal involvement owing to hypoxia and toxemia.

Salmonella infection is currently treated with antibiotics; however, some strains of salmonella are quickly developing antibiotic resistance. Currently, there is no vaccine available to prevent this bacterial infection. The present disclosure provides RNA (e.g., mRNA) vaccines against Salmonella infection—vaccines that elicit potent neutralizing antibodies and/or robust T cell responses against Salmonella antigens.

The vaccines disclosed herein may also be used therapeutically, i.e., following infection with Salmonella (to treat the infection). RNA vaccines disclosed herein have been demonstrated to result in expression of Salmonella proteins in eukaryotic cells and can induce an immune response in an animal model, as disclosed in the Examples section.

The Salmonella RNA vaccines described herein are superior to current vaccines in several ways. For example, the lipid nanoparticle (LNP) delivery system used herein increases the efficacy of RNA vaccines in comparison to other formulations, including a protamine-based approach described in the literature. The use of this LNP delivery system enables the effective delivery of chemically-modified RNA vaccines or unmodified RNA vaccines, without requiring additional adjuvant to produce a therapeutic result (e.g., production neutralizing antibody titer and/or a T cell response). In some embodiments, the Salmonella RNA vaccines disclosed herein are superior to conventional vaccines by a factor of at least 10 fold, 20, fold, 40, fold, 50 fold, 100 fold, 500 fold, or 1,000 fold when administered intramuscularly (IM) or intradermally (ID). These results can be achieved even when significantly lower doses of the RNA (e.g., mRNA) are administered in comparison with RNA doses used in other classes of lipid based formulations. These results are surprising because even at the very low doses tested, administration of the Salmonella RNA vaccines of the present disclosure results in appropriate expression of bacterial antigens in eukaryotic cells of the host and the induction of neutralizing immunity.

### Exemplary Salmonella Antigens

Antigens are proteins capable of inducing an immune response (e.g., causing an immune system to produce antibodies against the antigens). Herein, use of the term antigen encompasses immunogenic proteins and immunogenic fragments (an immunogenic fragment that induces (or is capable of inducing) an immune response to Salmonella), unless otherwise stated. It should be understood that the term “protein” encompasses peptides and the term “antigen” encompasses antigenic fragments.

A number of different antigens are associated with Salmonella. Salmonella vaccines, as provided herein, comprise at least one (one or more) ribonucleic acid (RNA, e.g., mRNA) polynucleotide having an open reading frame encoding at least one Salmonella antigen. Non-limiting examples of Salmonella antigens are provided below.

Exemplary Salmonella antigens are provided in the Sequence Listing elsewhere herein. For example, the antigens may be encoded by (thus the RNA may comprise or consist of) any one of sequences set forth in SEQ ID NO: 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, and/or 234. In some embodiments, the aforementioned sequences may further comprise a 5′ cap (e.g., 7mG(5′)ppp(5′)NlmpNp), a polyA tail, or a 5′ cap and a polyA tail.

Secreted effector protein, SseB, alters host cell physiology and promotes bacterial survival in host tissues. It is required for the correct localization of SseC and SseD on the bacterial cell surface (Nikolaus et al., J. Bacteriolo. 183:6036-605 (2001)). In some embodiments, a Salmonella vaccine of the present disclosure comprises a RNA (e.g., mRNA) encoding a SseB antigen. In some embodiments, the Salmonella SseB antigen comprises the sequence identified by SEQ ID NO: 25 (St_SseB) or SEQ ID NO: 20 (St_SseB_nIgK), or a sequence at least 90% identical to the sequence identified by SEQ ID NO: 25 or SEQ ID NO: 20. In some embodiments, the Salmonella SseB antigen is encoded by the sequence identified by SEQ ID NO: 26 or SEQ ID NO: 30, or a sequence at least 90% identical to the sequence identified by SEQ ID NO: 26 or SEQ ID NO: 30.

Mig14 is a host-induced virulence gene in Salmonella typhi and S. typhimurium, and plays an important role in cell invasion, and may be involved in flagellation, motility and chemotaxis of the bacterium as well (Sheng et al., Res Microbiol., 164(9):903-912 (2013)). The inner membrane-associated protein, Mig14, provides resistance to cathelin-related anti-microbial peptide (CRAMP), which is highly expressed in activated macrophages, by preventing CRAMP from entering the inner membrane and therefore enhancing Salmonella survival (Brodsky et al., Mol Microbiol. 55(3): 954-972 (2005)). In some embodiments, the Salmonella Mig14 antigen comprises the sequence identified by SEQ ID NO: 55 (St_Mig14), SEQ ID NO: 59 (St_Mig14_nIgK), SEQ ID NO: 66 (St_Mig14_NGM) or SEQ ID NO: 67 (St_Mig14_NGM_nIgK), or a sequence at least 90% identical to the sequence identified by SEQ ID NO: 55, SEQ ID NO: 59, SEQ ID NO: 66 or SEQ ID NO: 67. In some embodiments, the Salmonella Mig14 antigen is encoded by the sequence identified by SEQ ID NO: 56, SEQ ID NO: 60, SEQ ID NO: 64 or SEQ ID NO: 68, or a sequence at least 90% identical to the sequence identified by SEQ ID NO: 56, SEQ ID NO: 60, SEQ ID NO: 64 or SEQ ID NO: 68.

Salmonella outer membrane proteins (omp) OmpL, OmpC, OmpD, and OmpF are highly immunogenic porins, which stimulate innate and adaptive immune responses in the absence of adjuvants. These porins also induce the expression of co-stimulatory molecules on antigen-presenting cells through toll-like receptor canonical signaling pathways. Furthermore, these porins induce the release of TNF-α, IL-6, and IL18 (Galdiero et al., Microbiol. 147: 2697-2704 (2001)) and regulate the expression of CD80 and CD86 molecules on B cells and macrophages (Galdiero et al., Clinical Microbiol and Infection, 9(11): 1104-1111 (2003)).

In some embodiments, the Salmonella OmpL antigen comprises the sequence identified by SEQ ID NO: 103 (St_OmpL_nIgK) or SEQ ID NO: 107 (St_OmpL_NGM_IgK), or a sequence at least 90% identical to the sequence identified by SEQ ID NO: 103 or SEQ ID NO: 107. In some embodiments, the Salmonella OmpL antigen is encoded by the sequence identified by SEQ ID NO: 104 or SEQ ID NO: 108, or a sequence at least 90% identical to the sequence identified by SEQ ID NO: 104 or SEQ ID NO: 108.

In some embodiments, the Salmonella OmpC antigen comprises the sequence identified by SEQ ID NO: 5 (St_OmpC_nIgK) or SEQ ID NO: 21 (St_OmpC_NGM_IgK), or a sequence at least 90% identical to the sequence identified by SEQ ID NO: 5 or SEQ ID NO: 21. In some embodiments, the Salmonella OmpC antigen is encoded by the sequence identified by SEQ ID NO: 6 or SEQ ID NO: 22, or a sequence at least 90% identical to the sequence identified by SEQ ID NO: 6 or SEQ ID NO: 22.

In some embodiments, the Salmonella OmpD antigen comprises the sequence identified by SEQ ID NO: 17 (St_OmpD_nIgK_variant), or a sequence at least 90% identical to the sequence identified by SEQ ID NO: 17. In some embodiments, the Salmonella OmpD antigen is encoded by the sequence identified by SEQ ID NO: 18, or a sequence at least 90% identical to the sequence identified by SEQ ID NO: 18.

In some embodiments, the Salmonella OmpF antigen comprises the sequence identified by SEQ ID NO: 9 (St_OmpF_nIgK_variant) or SEQ ID NO: 13 (St_OmpF_NGM_IgK), or a sequence at least 90% identical to the sequence identified by SEQ ID NO: 9 or SEQ ID NO: 13. In some embodiments, the Salmonella OmpF antigen is encoded by the sequence identified by SEQ ID NO: 10 or SEQ ID NO: 14, or a sequence at least 90% identical to the sequence identified by SEQ ID NO: 10 or SEQ ID NO: 14.

It should be understood that while the foregoing Salmonella Omp antigens identified by SEQ ID NOs: 103, 107, 5, 21, 17, 9, or 13, or the RNA ORFs identified by SEQ ID NOs: 104, 108, 6, 22, 18, 10, or 14 include an N-terminal IgK signal sequence (SEQ ID NO: 153) (encoded by SEQ ID NO: 157)), the scope of the present disclosure also encompasses Omp antigens without the N-terminal IgK signal sequence or with an alternative signal sequence, such as any of those provided herein (e.g., SEQ ID NOs: 151, 152, 154, 155 or 156).

FepA (ferrienterobactin receptor), IroN, and CirA are iron-regulated outer membrane proteins (IROMPs) located on Salmonella enterica serovar typhimurium. The proteins are implicated in the uptake of enterobactin and may increase the capability of the bacterium to obtain iron using siderophore piracy (Rabsch et al., J of Bacteriol. 181(11):3610-3612 (1999)). The three are catecholate receptors; FepA and IroN are required for the transport of enterobactin, while all three proteins are receptors for 2,3-dihydroxybenzoylserine, an enterobactin breakdown product, which appears to play an important role in the virulence of Salmonella enterica (Rabsch et al., Infection and Immunity, 71(2):6953-6961 (2003)).

In some embodiments, the Salmonella FepA antigen comprises the sequence identified by SEQ ID NO: 91 (St_FepA_nIgK) or SEQ ID NO: 95 (St_FepA_NGM_nIgK), or a sequence at least 90% identical to the sequence identified by SEQ ID NO: 91 or SEQ ID NO: 95. In some embodiments, the Salmonella FepA antigen is encoded by the sequence identified by SEQ ID NO: 92 or SEQ ID NO: 96, or a sequence at least 90% identical to the sequence identified by SEQ ID NO: 92 or SEQ ID NO: 96. It should be understood that while the foregoing Salmonella FepA antigens identified by SEQ ID NOs: 91 or 95, or the RNA ORFs identified by SEQ ID NOs: 92 or 96 include an N-terminal IgK signal sequence (SEQ ID NO: 153) (encoded by SEQ ID NO: 157)), the scope of the present disclosure also encompasses FepA antigens without the N-terminal IgK signal sequence or with an alternative signal sequence, such as any of those provided herein (e.g., SEQ ID NOs: 151, 152, 154, 155 or 156).

In some embodiments, the Salmonella IroN antigen comprises the sequence identified by SEQ ID NO: 73 (St_IroN_nFLRT2) or SEQ ID NO: 77 (St_IroN_NGM_nFLRT2), or a sequence at least 90% identical to the sequence identified by SEQ ID NO: 73 or SEQ ID NO: 77. In some embodiments, the Salmonella IroN antigen is encoded by the sequence identified by SEQ ID NO: 74 or SEQ ID NO: 78, or a sequence at least 90% identical to the sequence identified by SEQ ID NO: 74 or SEQ ID NO: 78. It should be understood that while the foregoing Salmonella IroN antigens identified by SEQ ID NOs: 73 or 77, or the RNA ORFs identified by SEQ ID NOs: 74 or 76 include an N-terminal FLRT2 signal sequence, the scope of the present disclosure also encompasses IroN antigens without the N-terminal FLRT2 signal sequence or with an alternative signal sequence, such as any of those provided herein (e.g., SEQ ID NOs: 151, 152, 154, 155 or 156).

In some embodiments, the Salmonella CirA antigen comprises the sequence identified by SEQ ID NO: 81 (St_CirA_nFLRT2) or SEQ ID NO: 85 (St_CirA_NGM_nFLRT2), or a sequence at least 90% identical to the sequence identified by SEQ ID NO: 81 or SEQ ID NO: 85. In some embodiments, the Salmonella CirA antigen is encoded by the sequence identified by SEQ ID NO: 82 or SEQ ID NO: 86, or a sequence at least 90% identical to the sequence identified by SEQ ID NO: 82 or SEQ ID NO: 86. It should be understood that while the foregoing Salmonella CirA antigens identified by SEQ ID NOs: 81 or 85, or the RNA ORFs identified by SEQ ID NOs: 82 or 86 include an N-terminal FLRT2 signal sequence, the scope of the present disclosure also encompasses CirA antigens without the N-terminal FLRT2 signal sequence or with an alternative signal sequence, such as any of those provided herein (e.g., SEQ ID NOs: 151, 152, 154, 155 or 156).

T0937 is expressed during infection and confers some level of protective immunity in a mouse model (Bumann, Frontiers in Immunol., 5(391):1-5 (2014)). In some embodiments, the Salmonella T0937 antigen comprises the sequence identified by SEQ ID NO: 132 (St_T0937_nIgK_NGM_cHis) or SEQ ID NO: 134 (St_T0937_nIgK_nTrunc_NGM_cHis), or a sequence at least 90% identical to the sequence identified by SEQ ID NO: 132 or SEQ ID NO: 134. In some embodiments, the Salmonella T0937 antigen is encoded by the sequence identified by SEQ ID NO: 133 or SEQ ID NO: 135, or a sequence at least 90% identical to the sequence identified by SEQ ID NO: 133 or SEQ ID NO: 135. It should be understood that while the foregoing Salmonella T0937 antigens identified by SEQ ID NOs: 132 or 134, or the RNA ORFs identified by SEQ ID NOs: 133 or 135 include an N-terminal IgK signal sequence (SEQ ID NO: 153) (encoded by SEQ ID NO: 157)) and a C-terminal His tag, the scope of the present disclosure also encompasses T0937 antigens without the N-terminal IgK signal sequence or with an alternative signal sequence, such as any of those provided herein (e.g., SEQ ID NOs: 151, 152, 154, 155 or 156) and/or without the C-terminal His tag.

FliC is an antigenic form of flagella, and polymerizes to form the filaments of Salmonella flagella. It is expressed on the surface of Salmonella and is the target of host immune responses, as its invasion or translocation across the intestinal epithelium stimulates the innate immune receptor TLR5 to initiate an inflammatory response, as well as adaptive immune responses through FliC induction of antibody responses (Cummings et al., Mol Microbiol., 61(3): 795-809 (2006)). In some embodiments, the Salmonella FliC antigen comprises the sequence identified by SEQ ID NO: 33 (St_FliC), SEQ ID NO: 37 (St_FliC_nIgK), SEQ ID NO: 41 (SpA_FliC), SEQ ID NO: 45 (SpA_FliC_nIgK), SEQ ID NO: 49 (Stm_FliC) or SEQ ID NO: 53 (Stm_FliC_nIgK), or a sequence at least 90% identical to the sequence identified by SEQ ID NO: 33, SEQ ID NO: 37, SEQ ID NO: 41, or SEQ ID NO: 53. In some embodiments, the Salmonella FliC antigen is encoded by the sequence identified by SEQ ID NO: 34, SEQ ID NO: 38, SEQ ID NO: 42 or SEQ ID NO: 54, or a sequence at least 90% identical to the sequence identified by SEQ ID NO: 34, SEQ ID NO: 38, SEQ ID NO: 42 or SEQ ID NO: 54. It should be understood that while the foregoing Salmonella FliC antigens identified by SEQ ID NOs: 33, 37, 41 or 45, or the RNA ORFs identified by SEQ ID NOs: 34, 38, 42 or 46 include an N-terminal IgK signal sequence (SEQ ID NO: 153) (encoded by SEQ ID NO: 157)), the scope of the present disclosure also encompasses FliC antigens without the N-terminal IgK signal sequence or with an alternative signal sequence, such as any of those provided herein (e.g., SEQ ID NOs: 151, 152, 154, 155 or 156).

Putative type IV pilus protein, PiIL, plays a role in the adhesion of Salmonella to INT407 cells in vitro, an interaction mediated directly instead of via aggregation (van Asten et al., FEMS Immunol & Med Microbiol., 44(3): 251-259 (2005)). The proteins are assembled in the inner membrane and moved through the periplasm to the outer membrane, where the pilus exits to the cell surface of the bacteria; however, the pilus remains connected to the inner membrane of the bacteria and can be retracted inside the bacteria, as necessary (Pan et al., Antimicrobial Agents and Chemotherapy, 49(10), 4052-4060 (2005)). In some embodiments, the Salmonella PiIL antigen comprises the sequence identified by SEQ ID NO: 127 (St_PiIL_nIgK) or SEQ ID NO: 130 (St_PiIL_NGM_nIgK), or a sequence at least 90% identical to the sequence identified by SEQ ID NO: 127 or SEQ ID NO: 130. In some embodiments, the Salmonella PiIL antigen is encoded by the sequence identified by SEQ ID NO: 128 or SEQ ID NO: 131, or a sequence at least 90% identical to the sequence identified by SEQ ID NO: 128 or SEQ ID NO: 131. It should be understood that while the foregoing Salmonella PiIL antigens identified by SEQ ID NOs: 127 or 130, or the RNA ORFs identified by SEQ ID NOs: 128 or 131 include an N-terminal IgK signal sequence (SEQ ID NO: 153) (encoded by SEQ ID NO: 157)), the scope of the present disclosure also encompasses PiIL antigens without the N-terminal IgK signal sequence or with an alternative signal sequence, such as any of those provided herein (e.g., SEQ ID NOs: 151, 152, 154, 155 or 156).

PltB, PltA, and CdtB together form a tripartite toxin, cytolethal distending toxin (CDT). While CdtB is the functional cytolethal distending toxin, PltB and PltA are homologs of subunits of the pertussis toxin and are necessary for the delivery of CdtB from an intracellular compartment to target cells through both paracrine and autocrine pathway (Spano et al., Cell Host and Microbe, 3(1): 30-338 (2008)). The toxin has both a DNase activity from CdtB as well as an ADP-ribosylating activity associated with PltA, and has been found in both typhoidal and nontyphoidal Salmonella serotypes (Miller et al., Toxins, 8(5): 121-140 (2016)). In some embodiments, a Salmonella vaccine of the present disclosure comprises a mRNA encoding a PtlB antigen, a PltA antigen and/or a CdtB antigen.

In some embodiments, the Salmonella PltB antigen comprises the sequence identified by SEQ ID NO: 119 (St_PltB_nIgK) or SEQ ID NO: 123 (St_PltB_NGM_nIgK), or a sequence at least 90% identical to the sequence identified by SEQ ID NO: 119 or SEQ ID NO: 123. In some embodiments, the Salmonella PltB antigen is encoded by the sequence identified by SEQ ID NO: 120 or SEQ ID NO: 124, or a sequence at least 90% identical to the sequence identified by SEQ ID NO: 120 or SEQ ID NO: 124. It should be understood that while the foregoing Salmonella PltB antigens identified by SEQ ID NOs: 119 or 123, or the RNA ORFs identified by SEQ ID NOs: 120 or 124 include an N-terminal IgK signal sequence (SEQ ID NO: 153) (encoded by SEQ ID NO: 157)), the scope of the present disclosure also encompasses PltB antigens without the N-terminal IgK signal sequence or with an alternative signal sequence, such as any of those provided herein (e.g., SEQ ID NOs: 151, 152, 154, 155 or 156).

In some embodiments, the Salmonella PltA antigen comprises the sequence identified by SEQ ID NO: 111 (St_PltA_nIgK) or SEQ ID NO: 115 (St_PltA_NGM_nIgK), or a sequence at least 90% identical to the sequence identified by SEQ ID NO: 111 or SEQ ID NO: 115. In some embodiments, the Salmonella PltA antigen is encoded by the sequence identified by SEQ ID NO: 112 or SEQ ID NO: 116, or a sequence at least 90% identical to the sequence identified by SEQ ID NO: 112 or SEQ ID NO: 116. It should be understood that while the foregoing Salmonella PltA antigens identified by SEQ ID NOs: 111 or 115, or the RNA ORFs identified by SEQ ID NOs: 112 or 116 include an N-terminal IgK signal sequence (SEQ ID NO: 153) (encoded by SEQ ID NO: 157)), the scope of the present disclosure also encompasses PltA antigens without the N-terminal IgK signal sequence or with an alternative signal sequence, such as any of those provided herein (e.g., SEQ ID NOs: 151, 152, 154, 155 or 156).

In some embodiments, the Salmonella CdtB antigen comprises the sequence identified by SEQ ID NO: 97 (St_CdtB_nIgK), SEQ ID NO: 99 (St_CdtB_NGM_nIgK), SEQ ID NO: 138 (St_CdtB_Trunc_IgK_cHis) or SEQ ID NO: 141 (St_CdtB_Trunc_H160Q_IgK_cHis), or a sequence at least 90% identical to the sequence identified by SEQ ID NO: 97, SEQ ID NO: 99, SEQ ID NO: 138 or SEQ ID NO: 141. In some embodiments, the Salmonella CdtB antigen is encoded by the sequence identified by SEQ ID NO: 98, SEQ ID NO: 100, SEQ ID NO: 139 or SEQ ID NO: 142, or a sequence at least 90% identical to the sequence identified by SEQ ID NO: 98, SEQ ID NO: 100, SEQ ID NO: 139 or SEQ ID NO: 142. It should be understood that while the foregoing Salmonella CdtB antigens identified by SEQ ID NOs: 97, 99, 138 or 141, or the RNA ORFs identified by SEQ ID NOs: 98, 100, 139 or 142 include an N-terminal IgK signal sequence (SEQ ID NO: 153) (encoded by SEQ ID NO: 157)) or a C-terminal His tag, the scope of the present disclosure also encompasses CdtB antigens without the N-terminal IgK signal sequence or with an alternative signal sequence, such as any of those provided herein (e.g., SEQ ID NOs: 151, 152, 154, 155 or 156) and/or without the C-terminal His tag.

SlyB, an outer membrane lipoprotein, is another Salmonella antigen. It negatively regulates PhoP activity and is essential to the PhoP/PhoQ system, which dictates the expression of Mg²⁺ transporters and enzymes that alter Mg²⁺ binding sites (Perez et al., PLOS Genetics, 5(3):e10000428 (2009)). In some embodiments, a Salmonella vaccine of the present disclosure comprises a mRNA encoding a SlyB antigen. In some embodiments, the Salmonella SlyB antigen comprises the sequence identified by SEQ ID NO: 136 (St_slyB_nIgK_NGM_cHis), or a sequence at least 90% identical to the sequence identified by SEQ ID NO: 136. In some embodiments, the Salmonella CdtB antigen is encoded by the sequence identified by SEQ ID NO: 137, or a sequence at least 90% identical to the sequence identified by SEQ ID NO: 137. It should be understood that while the foregoing Salmonella SlyB antigen identified by SEQ ID NO: 136, or the RNA ORF identified by SEQ ID NO: 137 includes an N-terminal IgK signal sequence (SEQ ID NO: 153) (encoded by SEQ ID NO: 157)) or a C-terminal His tag, the scope of the present disclosure also encompasses SlyB antigens without the N-terminal IgK signal sequence or with an alternative signal sequence, such as any of those provided herein (e.g., SEQ ID NOs: 151, 152, 154, 155 or 156) and/or without the C-terminal His tag.

STY1086 is a putative lipoprotein found on the cell surface of Salmonella (Thieu et al., J of Infection, 75: 104-114 (2017)). In some embodiments, a Salmonella vaccine of the present disclosure comprises a mRNA encoding a STY1086 antigen. In some embodiments, the Salmonella STY1086 antigen comprises the sequence identified by SEQ ID NO: 147 (St_STY1086_nIgK_cHis) or SEQ ID NO: 149 (St_STY1086_NGM_nIgK_cHis), or a sequence at least 90% identical to the sequence identified by SEQ ID NO: 147 or SEQ ID NO: 149. In some embodiments, the Salmonella STY1086 antigen is encoded by the sequence identified by SEQ ID NO: 148 or SEQ ID NO: 150, or a sequence at least 90% identical to the sequence identified by SEQ ID NO: 148 or SEQ ID NO: 150. It should be understood that while the foregoing Salmonella STY1086 antigen identified by SEQ ID NOs: 147 or 149, or the RNA ORF identified by SEQ ID NOs: 148 or 150 include an N-terminal IgK signal sequence (SEQ ID NO: 153) (encoded by SEQ ID NO: 157)) or a C-terminal His tag, the scope of the present disclosure also encompasses STY1086 antigens without the N-terminal IgK signal sequence or with an alternative signal sequence, such as any of those provided herein (e.g., SEQ ID NOs: 151, 152, 154, 155 or 156) and/or without the C-terminal His tag.

STY0796 (cell divisional coordinator CpoB) is a putative exported protein and is part of the Tol/pal system. It interacts with TolA and is involved in maintaining cell envelope integrity, including mediating the coordination of peptidoglycan synthesis and outer membrane constriction during division (Thieu et al., J of Infection, 75: 104-114 (2017)). In some embodiments, a Salmonella vaccine of the present disclosure comprises a mRNA encoding a STY0796 antigen. In some embodiments, the Salmonella STY0796 antigen comprises the sequence identified by SEQ ID NO: 143 (St_STY0796_nIgK_cHis) or SEQ ID NO: 145 (St_STY0796_NGM_nIgK_cHis), or a sequence at least 90% identical to the sequence identified by SEQ ID NO: 143 or SEQ ID NO: 145. In some embodiments, the Salmonella STY0796 antigen is encoded by the sequence identified by SEQ ID NO: 144 or SEQ ID NO: 146, or a sequence at least 90% identical to the sequence identified by SEQ ID NO: 144 or SEQ ID NO: 146. It should be understood that while the foregoing Salmonella STY1086 antigen identified by SEQ ID NOs: 143 or 145, or the RNA ORF identified by SEQ ID NOs: 144 or 146 include an N-terminal IgK signal sequence (SEQ ID NO: 153) (encoded by SEQ ID NO: 157)) or a C-terminal His tag, the scope of the present disclosure also encompasses STY0796 antigens without the N-terminal IgK signal sequence or with an alternative signal sequence, such as any of those provided herein (e.g., SEQ ID NOs: 151, 152, 154, 155 or 156) and/or without the C-terminal His tag.

### Nucleic Acids

The Salmonella vaccines of the present disclosure comprise at least one (one or more) ribonucleic acid (RNA) having an open reading frame encoding at least one Salmonella antigen. In some embodiments, the RNA is a messenger RNA (mRNA) having an open reading frame encoding at least one Salmonella antigen. In some embodiments, the RNA (e.g., mRNA) further comprises a 5′ UTR, 3′ UTR, a polyA tail and/or a 5′ cap.

Nucleic acids comprise a polymer of nucleotides (nucleotide monomers), also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof.

Messenger RNA (mRNA) is any ribonucleic acid that encodes a (at least one) protein (a naturally-occurring, non-naturally-occurring, or modified polymer of amino acids) and can be translated to produce the encoded protein in vitro, in vivo, in situ or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in a vaccine of the present disclosure.

### Variants

In some embodiments, an RNA of the present disclosure encodes a Salmonella antigen variant. Antigen or other polypeptide variants refers to molecules that differ in their amino acid sequence from a wild-type, native or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments, a Salmonella vaccine comprises an mRNA ORF having a nucleotide sequence identified by any one of the sequences provided herein (see e.g., Sequence Listing and Tables 1-9 of the Examples section), or having a nucleotide sequence at least 80%, at least 85%, at least 90%, at least 95%, at least 96%, at least 97%, at least 98%, or at least 99% identical to a nucleotide sequence identified by any one of the sequence provided herein.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA) vaccine.

As recognized by those skilled in the art, protein fragments, functional protein domains, and homologous proteins are also considered to be within the scope of Salmonella antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the Salmonella pathogen. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

### Stabilizing Elements

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a vaccine includes at least one RNA polynucleotide having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, a vaccine includes at least one RNA polynucleotide having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

In some embodiments, Salmonella RNA vaccines may include one or more stabilizing elements. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, Salmonella RNA vaccines include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, Salmonella RNA vaccines do not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

In some embodiments, Salmonella RNA vaccines may or may not contain an enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, Salmonella RNA vaccines may have one or more AU-rich sequences removed. These sequences, sometimes referred to as AURES are destabilizing sequences found in the 3′UTR. The AURES may be removed from the RNA vaccines. Alternatively the AURES may remain in the RNA vaccine.

### Signal Peptides

In some embodiments, a Salmonella vaccine comprises a RNA having an ORF that encodes a signal peptide fused to the Salmonella antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by a ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than Salmonella antigens in nature) are known in the art and can be tested for desired properties and then incorporated into a nucleic acid of the disclosure. In some embodiments, the signal peptide is a bovine prolactin signal peptide. For example, the bovine prolactin signal peptide may comprise sequence MDSKGSSQKGSRLLLLLVVSNLLLPQGVVG (SEQ ID NO:151). Other signal peptide sequences that may be used as provided herein include, without limitation, MDWTWILFLVAAATRVHS (SEQ ID NO: 152), METPAQLLFLLLLWLPDTTG (SEQ ID NO:1 53), MLGSNSGQRVVFTILLLLVAPAYS (SEQ ID NO: 154), MKCLLYLAFLFIGVNCA (SEQ ID NO: 155), and MWLVSLAIVTACAGA (SEQ ID NO: 156).

### Fusion Proteins

In some embodiments, a Salmonella RNA vaccine of the present disclosure includes an RNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the Salmonella antigen. Antigenic fusion proteins, in some embodiments, retain the functional property from each original protein.

### Scaffold Moieties

The RNA (e.g., mRNA) vaccines as provided herein, in some embodiments, encode fusion proteins which comprise Salmonella antigens linked to scaffold moieties. In some embodiments, such scaffold moieties impart desired properties to an antigen encoded by a nucleic acid of the disclosure. For example scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of ˜22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 Å and 360 Å diameter, corresponding to 180 or 240 protomers. In some embodiments a Salmonella antigen is fused to HBsAG or HBcAG to facilitate self-assembly of nanoparticles displaying the Salmonella antigen.

In other embodiments, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The LS monomer is 150 amino acids long, and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 Å diameter. Even LS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

### Linkers and Cleavable Peptides

In some embodiments, the mRNAs of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6: e18556). In some embodiments, the linker is an F2A linker. In some embodiments, the linker is a GGGS linker. In some embodiments, the fusion protein contains three domains with intervening linkers, having the structure: domain-linker-domain-linker-domain.

Cleavable linkers known in the art may be used in connection with the disclosure. Exemplary such linkers include: F2A linkers, T2A linkers, P2A linkers, E2A linkers (See, e.g., WO2017127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park Calif.) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a Salmonella antigen). In some embodiments, a codon optimized sequence shares less than 90% sequence identity to a naturally-occurring or wild-type sequence (e.g., a naturally-occurring or wild-type mRNA sequence encoding a Salmonella antigen). In some embodiments, a codon optimized sequence shares less than 85% sequence identity to a naturally-occurring or wild-type sequence (e.g., a naturally-occurring or wild-type mRNA sequence encoding a Salmonella antigen). In some embodiments, a codon optimized sequence shares less than 80% sequence identity to a naturally-occurring or wild-type sequence (e.g., a naturally-occurring or wild-type mRNA sequence encoding a Salmonella antigen). In some embodiments, a codon optimized sequence shares less than 75% sequence identity to a naturally-occurring or wild-type sequence (e.g., a naturally-occurring or wild-type mRNA sequence encoding a Salmonella antigen).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a Salmonella antigen). In some embodiments, a codon optimized sequence shares between 65% and 75% or about 80% sequence identity to a naturally-occurring or wild-type sequence (e.g., a naturally-occurring or wild-type mRNA sequence encoding a Salmonella antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a Salmonella antigen encoded by a non-codon-optimized)sequence.

When transfected into mammalian cells, the modified mRNAs have a stability of between 12-18 hours, or greater than 18 hours, e.g., 24, 36, 48, 60, 72, or greater than 72 hours.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, at least one RNA (e.g., mRNA) of a Salmonella vaccines of the present disclosure is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemically Unmodified Nucleotides

In some embodiments, at least one RNA (e.g., mRNA) of a almonella vaccines of the present disclosure is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

Salmonella RNA vaccines of the present disclosure comprise, in some embodiments, at least one nucleic acid (e.g., RNA) having an open reading frame encoding at least one Salmonella antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises 1-methyl-pseudouridine (m1ψ) substitutions at one or more or all uridine positions of the nucleic acid.

In some embodiments, a RNA nucleic acid of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Untranslated Regions (UTRs)

The nucleic acids of the present disclosure may comprise one or more regions or parts which act or function as an untranslated region. Where nucleic acids are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 159), where R is a purine (adenine or guanine) three bases upstream of the start codon (AUG), which is followed by another ‘G’.5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US20140206753, WO2013/185069), the sequence GGGAUCCUACC (SEQ ID NO: 236) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738), 5′ UTR element derived from the 5′UTR of an hydroxysteroid (17-β) dehydrogenase 4 gene (HSD17B4) (WO2015024667), or a 5′ UTR element derived from the 5′ UTR of ATP5A1 (WO2015024667) can be used. In one embodiment, an internal ribosome entry site (IRES) is used instead of a 5′ UTR.

In some embodiments, a 5′ UTR of the present disclosure comprises a sequence selected from SEQ ID NO:3 and SEQ ID NO:140.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) (SEQ ID NO: 160) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

3′ UTRs may be heterologous or synthetic. With respect to 3′ UTRs, globin UTRs, including Xenopus β-globin UTRs and human β-globin UTRs are known in the art (U.S. Pat. Nos. 8,278,063, 9,012,219, US20110086907). A modified β-globin construct with enhanced stability in some cell types by cloning two sequential human β-globin 3′UTRs head to tail has been developed and is well known in the art (US2012/0195936, WO2014/071963). In addition a2-globin, a1-globin, UTRs and mutants thereof are also known in the art (WO2015101415, WO2015024667). Other 3′ UTRs described in the mRNA constructs in the non-patent literature include CYBA (Ferizi et al., 2015) and albumin (Thess et al., 2015). Other exemplary 3′ UTRs include that of bovine or human growth hormone (wild type or modified) (WO2013/185069, US20140206753, WO2014152774), rabbit β globin and hepatitis B virus (HBV), α-globin 3′ UTR and Viral VEEV 3′ UTR sequences are also known in the art. In some embodiments, the sequence UUUGAAUU (WO2014144196) is used. In some embodiments, 3′ UTRs of human and mouse ribosomal protein are used. Other examples include rps9 3′UTR (WO2015101414), FIG. 4 (WO2015101415), and human albumin 7 (WO2015101415).

In some embodiments, a 3′ UTR of the present disclosure comprises a sequence selected from SEQ ID NO:4 and SEQ ID NO:129,

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3″ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

### In Vitro Transcription of RNA

cDNA encoding the polynucleotides described herein may be transcribed using an in vitro transcription (IVT) system. In vitro transcription of RNA is known in the art and is described in International Publication WO/2014/152027, which is incorporated by reference herein in its entirety.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to Salmonella RNA, e.g. Salmonella mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

### Chemical Synthesis

Solid-Phase Chemical Synthesis.

Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis.

The synthesis of nucleic acids of the present disclosure by the sequential addition of monomer building blocks may be carried out in a liquid phase.

Combination of Synthetic Methods.

The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, Mass.), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present invention may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, Mass.). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Pharmaceutical Formulations

Provided herein are compositions (e.g., pharmaceutical compositions), methods, kits and reagents for prevention or treatment of Salmonella in humans and other mammals, for example. Salmonella RNA (e.g., mRNA) vaccines can be used as therapeutic or prophylactic agents. They may be used in medicine to prevent and/or treat infectious disease.

In some embodiments, a Salmonella vaccine containing RNA polynucleotides as described herein can be administered to a subject (e.g., a mammalian subject, such as a human subject), and the RNA polynucleotides are translated in vivo to produce an antigenic polypeptide (antigen).

An “effective amount” of a Salmonella vaccine is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of a Salmonella vaccine provides an induced or boosted immune response as a function of antigen production in the cell. In some embodiments, an effective amount of the Salmonella RNA vaccine containing RNA polynucleotides having at least one chemical modifications are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, RNA vaccines (including polynucleotides and their encoded polypeptides) in accordance with the present disclosure may be used for treatment or prevention of Salmonella. Salmonella RNA vaccines may be administered prophylactically or therapeutically as part of an active immunization scheme to healthy individuals or early in infection during the incubation phase or during active infection after onset of symptoms. In some embodiments, the amount of RNA vaccines of the present disclosure provided to a cell, a tissue or a subject may be an amount effective for immune prophylaxis.

Salmonella RNA (e.g., mRNA) vaccines may be administrated with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, Salmonella RNA vaccines may be administered intramuscularly, intranasally or intradermally, similarly to the administration of inactivated vaccines known in the art.

The Salmonella RNA vaccines may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

Provided herein are pharmaceutical compositions including Salmonella RNA vaccines and RNA vaccine compositions and/or complexes optionally in combination with one or more pharmaceutically acceptable excipients.

Salmonella RNA (e.g., mRNA) vaccines may be formulated or administered alone or in conjunction with one or more other components. For instance, Salmonella RNA vaccines (vaccine compositions) may comprise other components including, but not limited to, adjuvants.

In some embodiments, Salmonella RNA vaccines do not include an adjuvant (they are adjuvant free).

Salmonella RNA (e.g., mRNA) vaccines may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, Salmonella RNA vaccines are administered to humans, human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigens.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, Salmonella RNA vaccines are formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with Salmonella RNA vaccines (e.g., for transplantation into a subject), hyaluronidase, nanoparticle mimics and combinations thereof.

### Lipid Nanoparticles (LNPs)

In some embodiments, Salmonella RNA (e.g., mRNA) vaccines of the disclosure are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, an ionizable cationic lipid of the disclosure comprises a compound of Formula (I):

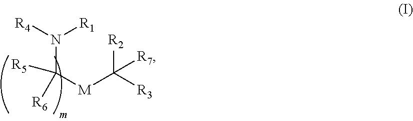

or a salt or isomer thereof, wherein:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆

carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)O R, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—,

—N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆

carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR,
—O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆

carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR,
—O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆

carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR,
—O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is —(CH₂)ₙQ or —(CH₂)ₙCHQR, where Q is —N(R)₂, and n is selected from 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13, or salts or isomers thereof.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or isomers thereof.

In some embodiments, a subset of compounds of Formula (I) includes those of Formula (IA):

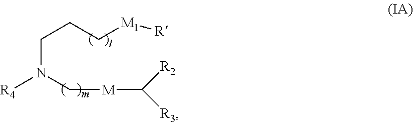

or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

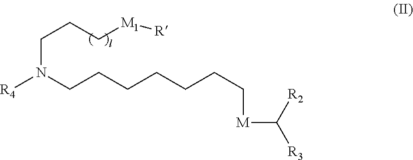

or a salt or isomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is
OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

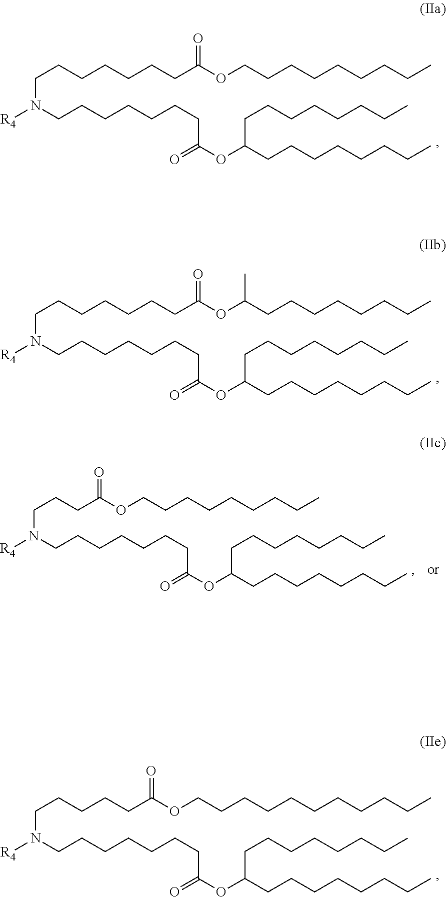

or a salt or isomer thereof, wherein R₄ is as described herein.

In some embodiments, a subset of compounds of Formula (I) includes those of Formula (IId):

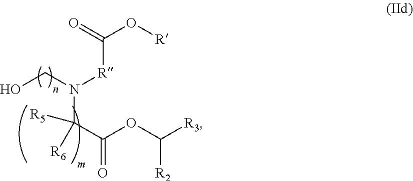

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable cationic lipid of the disclosure comprises a compound having structure:

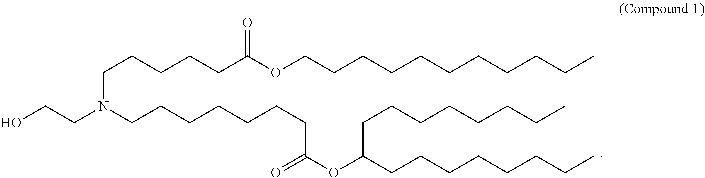 [SMILES: CCCCCCCCCCCOC(=O)CCCCCN(CCO)CCCCCCCC(=O)OC(CCCCCCCC)CCCCCCCC]

In some embodiments, an ionizable cationic lipid of the disclosure comprises a compound having structure:

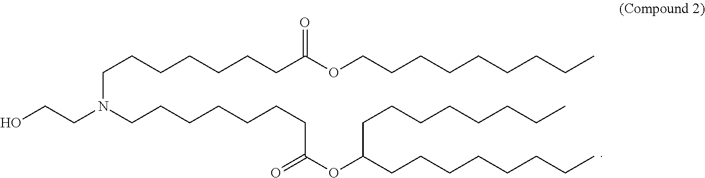 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, bras sicasterol, tomatidine, ursolic acid, alpha-tocopherol, and mixtures thereof.

In some embodiments, a LNP of the disclosure comprises an ionizable cationic lipid of Compound 1, wherein the non-cationic lipid is DSPC, the structural lipid that is cholesterol, and the PEG lipid is PEG-DMG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

### Multivalent Vaccines

The Salmonella vaccines, as provided herein, may include an RNA (e.g. mRNA) or multiple RNAs encoding two or more antigens of the same Salmonella species. In some embodiments, a Salmonella vaccine includes an RNA or multiple RNAs encoding two or more antigens selected from SseB, Mig14, OmpL, OmpC, OmpD, OmpF, IroN, CirA, FepA, T0937, FliC, PilL, PltB, PltA, CdtB, SlyB, STY1086 and STY0796 antigens. In some embodiments, the RNA (at least one RNA) of a Salmonella vaccine may encode 1, 2, 3, 4, 5, 6, 7, 8, 9, 10, 11, 12, or more antigens.

In some embodiments, a Salmonella vaccine comprises at least one RNA encoding a SseB antigen and a Mig14 antigen.

In some embodiments, a Salmonella vaccine comprises at least one RNA encoding a OmpL, OmpC, OmpD, and OmpF antigens.

In some embodiments, a Salmonella vaccine comprises at least one RNA encoding IroN, CirA, and FepA antigens.

In some embodiments, a Salmonella vaccine comprises at least one RNA encoding a PltA, a PltB and a CdtB antigen (which make up the toxin, e.g., in mutated form). In some embodiments, a Salmonella vaccine comprises at least one RNA encoding a PltA, a PltB, and a CdtB antigen and an additional Salmonella antigen. In some embodiments, a Salmonella vaccine comprises at least one RNA encoding a PltA, a PltB, a CdtB and a SseB antigen. In some embodiments, a Salmonella vaccine comprises at least one RNA encoding a PltA, a PltB, a CdtB and a Mig14 antigen. In some embodiments, a Salmonella vaccine comprises at least one RNA encoding a PltA, a PltB, a CdtB and a OmpL antigen. In some embodiments, a Salmonella vaccine comprises at least one RNA encoding a PltA, a PltB, a CdtB and a OmpC antigen. In some embodiments, a Salmonella vaccine comprises at least one RNA encoding a PltA, a PltB, a CdtB and a OmpD antigen. In some embodiments, a Salmonella vaccine comprises at least one RNA encoding a PltA, a PltB, a CdtB and a OmpF antigen. In some embodiments, a Salmonella vaccine comprises at least one RNA encoding a PltA, a PltB, a CdtB and an IroN antigen. In some embodiments, a Salmonella vaccine comprises at least one RNA encoding a PltA, a PltB, a CdtB and a CirA antigen. In some embodiments, a Salmonella vaccine comprises at least one RNA encoding a PltA, a PltB, a CdtB and a FepA antigen. In some embodiments, a Salmonella vaccine comprises at least one RNA encoding a PltA, a PltB, a CdtB and a T0937 antigen. In some embodiments, a Salmonella vaccine comprises at least one RNA encoding a PltA, a PltB, a CdtB and a FliC antigen. In some embodiments, a Salmonella vaccine comprises at least one RNA encoding a PltA, a PltB, a CdtB and a PilL antigen. In some embodiments, a Salmonella vaccine comprises at least one RNA encoding a PltA, a PltB, a CdtB and a SlyB antigen. In some embodiments, a Salmonella vaccine comprises at least one RNA encoding a PltA, a PltB, a CdtB and a STY1086 antigen. In some embodiments, a Salmonella vaccine comprises at least one RNA encoding a PltA, a PltB, a CdtB and a STY0796 antigen.

In some embodiments, two or more different RNA (e.g., mRNA) encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately.

### Combination Vaccines

The Salmonella vaccines, as provided herein, may include an RNA or multiple RNAs encoding two or more antigens of the same or different Salmonella species. Also provided herein are combination vaccines that include RNA encoding one or more Salmonella antigen(s) and one or more antigen(s) of a different organisms (e.g., bacterial and/or viral organism). Thus, the vaccines of the present disclosure may be combination vaccines that target one or more antigens of the same species, or one or more antigens of different species, e.g., antigens which induce immunity to organisms which are found in the same geographic areas where the risk of Salmonella infection is high or organisms to which an individual is likely to be exposed to when exposed to Salmonella.

### Dosing/Administration

Provided herein are compositions (e.g., pharmaceutical compositions), methods, kits and reagents for prevention and/or treatment of Salmonella in humans and other mammals. Salmonella RNA vaccines can be used as therapeutic or prophylactic agents. In some aspects, the RNA vaccines of the disclosure are used to provide prophylactic protection from Salmonella. In some aspects, the RNA vaccines of the disclosure are used to treat a Salmonella infection. In some embodiments, the Salmonella vaccines of the present disclosure are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, the Salmonella vaccines are administered to a subject (e.g., a mammalian subject, such as a human subject) in an effective amount to induce an antigen-specific immune response. The RNA encoding the Salmonella antigen is expressed and translated in vivo to produce the antigen, which then stimulates an immune response in the subject.

Prophylactic protection from Salmonella can be achieved following administration of a Salmonella RNA vaccine of the present disclosure. Vaccines can be administered once, twice, three times, four times or more but it is likely sufficient to administer the vaccine once (optionally followed by a single booster). It is possible, although less desirable, to administer the vaccine to an infected individual to achieve a therapeutic response. Dosing may need to be adjusted accordingly.

A method of eliciting an immune response in a subject against Salmonella is provided in aspects of the present disclosure. The method involves administering to the subject a Salmonella RNA vaccine comprising at least one RNA (e.g., mRNA) having an open reading frame encoding at least one Salmonella antigen, thereby inducing in the subject an immune response specific to Salmonella antigen, wherein anti-antigen antibody titer in the subject is increased following vaccination relative to anti-antigen antibody titer in a subject vaccinated with a prophylactically effective dose of a traditional vaccine against the Salmonella. An “anti-antigen antibody” is a serum antibody the binds specifically to the antigen.

A prophylactically effective dose is an effective dose that prevents infection with the bacteria at a clinically acceptable level. In some embodiments, the effective dose is a dose listed in a package insert for the vaccine. A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against the Salmonella or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against the Salmonella or an unvaccinated subject.

A method of eliciting an immune response in a subject against a Salmonella is provided in other aspects of the disclosure. The method involves administering to the subject a Salmonella RNA vaccine comprising at least one RNA polynucleotide having an open reading frame encoding at least one Salmonella antigen, thereby inducing in the subject an immune response specific to Salmonella antigen, wherein the immune response in the subject is equivalent to an immune response in a subject vaccinated with a traditional vaccine against the Salmonella at 2 times to 100 times the dosage level relative to the RNA vaccine.

In some embodiments, the immune response in the subject is equivalent to an immune response in a subject vaccinated with a traditional vaccine at twice the dosage level relative to the Salmonella RNA vaccine. In some embodiments, the immune response in the subject is equivalent to an immune response in a subject vaccinated with a traditional vaccine at three times the dosage level relative to the Salmonella RNA vaccine. In some embodiments, the immune response in the subject is equivalent to an immune response in a subject vaccinated with a traditional vaccine at 4 times, 5 times, 10 times, 50 times, or 100 times the dosage level relative to the Salmonella RNA vaccine. In some embodiments, the immune response in the subject is equivalent to an immune response in a subject vaccinated with a traditional vaccine at 10 times to 1000 times the dosage level relative to the Salmonella RNA vaccine. In some embodiments, the immune response in the subject is equivalent to an immune response in a subject vaccinated with a traditional vaccine at 100 times to 1000 times the dosage level relative to the Salmonella RNA vaccine.

In other embodiments, the immune response is assessed by determining [protein] antibody titer in the subject.

Other aspects the disclosure provide methods of eliciting an immune response in a subject against a Salmonella by administering to the subject a Salmonella RNA vaccine comprising at least one RNA polynucleotide having an open reading frame encoding at least one Salmonella antigen, thereby inducing in the subject an immune response specific to Salmonella antigen, wherein the immune response in the subject is induced 2 days to 10 weeks earlier relative to an immune response induced in a subject vaccinated with a prophylactically effective dose of a traditional vaccine against the Salmonella. In some embodiments, the immune response in the subject is induced in a subject vaccinated with a prophylactically effective dose of a traditional vaccine at 2 times to 100 times the dosage level relative to the RNA vaccine.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against a Salmonella by administering to the subject a Salmonella RNA vaccine having an open reading frame encoding a first antigen, wherein the RNA polynucleotide does not include a stabilization element, and wherein an adjuvant is not co-formulated or co-administered with the vaccine.

Salmonella RNA (e.g., mRNA) vaccines may be administered by any route which results in a therapeutically effective outcome. These include, but are not limited, to intradermal, intramuscular, intranasal, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. Salmonella RNA (e.g., mRNA) vaccines compositions are typically formulated in dosage unit form for ease of administration and uniformity of dosage. It will be understood, however, that the total daily usage of Salmonella RNA (e.g., mRNA)vaccines compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of a Salmonella vaccine, as provided herein, may be as low as 20 μg, administered for example as a single dose or as two 10 μg doses. In some embodiments, the effective amount is a total dose of 20 μg-200 μg. For example, the effective amount may be a total dose of 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg or 200 μg. In some embodiments, the effective amount is a total dose of 25 μg-200 μg. In some embodiments, the effective amount is a total dose of 50 μg-200 μg.

In some embodiments, Salmonella RNA (e.g., mRNA) vaccines compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No. WO2013078199, herein incorporated by reference in its entirety). The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, Salmonella RNA (e.g., mRNA) vaccines compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, Salmonella RNA (e.g., mRNA) vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, Salmonella RNA (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, a Salmonella RNA (e.g., mRNA) vaccine composition may be administered three or four times.

In some embodiments, Salmonella RNA (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments, the Salmonella RNA (e.g., mRNA) vaccine for use in a method of vaccinating a subject is administered the subject a single dosage of between 10 μg/kg and 400 μg/kg of the nucleic acid vaccine in an effective amount to vaccinate the subject. In some embodiments, the RNA vaccine for use in a method of vaccinating a subject is administered the subject a single dosage of between 10 μg and 400 μg of the nucleic acid vaccine in an effective amount to vaccinate the subject. In some embodiments, a Salmonella RNA (e.g., mRNA) vaccine for use in a method of vaccinating a subject is administered to the subject as a single dosage of 25-1000 μg (e.g., a single dosage of mRNA encoding an Salmonella antigen). In some embodiments, a Salmonella RNA vaccine is administered to the subject as a single dosage of 25, 50, 100, 150, 200, 250, 300, 350, 400, 450, 500, 550, 600, 650, 700, 750, 800, 850, 900, 950 or 1000 μg. For example, a Salmonella RNA vaccine may be administered to a subject as a single dose of 25-100, 25-500, 50-100, 50-500, 50-1000, 100-500, 100-1000, 250-500, 250-1000, or 500-1000 μg. In some embodiments, a Salmonella RNA (e.g., mRNA) vaccine for use in a method of vaccinating a subject is administered to the subject as two dosages, the combination of which equals 25-1000 μg of the Salmonella RNA (e.g., mRNA) vaccine.

A Salmonella RNA (e.g., mRNA) vaccine pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects of the present disclosure provide formulations of the Salmonella RNA (e.g., mRNA) vaccine, wherein the Salmonella RNA vaccine is formulated in an effective amount to produce an antigen specific immune response in a subject (e.g., production of antibodies specific to an anti-Salmonella antigen). “An effective amount” is a dose of an Salmonella RNA (e.g., mRNA) vaccine effective to produce an antigen-specific immune response. Also provided herein are methods of inducing an antigen-specific immune response in a subject.

As used herein, an immune response to a vaccine or LNP of the present invention is the development in a subject of a humoral and/or a cellular immune response to a (one or more) Salmonella protein(s) present in the vaccine. For purposes of the present invention, a “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function, and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells.

In some embodiments, the antigen-specific immune response is characterized by measuring an anti-Salmonella antigen antibody titer produced in a subject administered a Salmonella RNA (e.g., mRNA) vaccine as provided herein. An antibody titer is a measurement of the amount of antibodies within a subject, for example, antibodies that are specific to a particular antigen (e.g., an anti-Salmonella antigen) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by the Salmonella RNA (e.g., mRNA) vaccine.

In some embodiments, an anti-Salmonella antigen antibody titer produced in a subject is increased by at least 1 log relative to a control. For example, anti-Salmonella antigen antibody titer produced in a subject may be increased by at least 1.5, at least 2, at least 2.5, or at least 3 log relative to a control. In some embodiments, the anti-Salmonella antigen antibody titer produced in the subject is increased by 1, 1.5, 2, 2.5 or 3 log relative to a control. In some embodiments, the anti-Salmonella antigen antibody titer produced in the subject is increased by 1-3 log relative to a control. For example, the anti-Salmonella antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-Salmonella antigen antibody titer produced in a subject is increased at least 2 times relative to a control. For example, the anti-Salmonella antigen antibody titer produced in a subject may be increased at least 3 times, at least 4 times, at least 5 times, at least 6 times, at least 7 times, at least 8 times, at least 9 times, or at least 10 times relative to a control. In some embodiments, the anti-Salmonella antigen antibody titer produced in the subject is increased 2, 3, 4, 5, 6, 7, 8, 9, or 10 times relative to a control. In some embodiments, the anti-Salmonella antigen antibody titer produced in a subject is increased 2-10 times relative to a control. For example, the anti-Salmonella antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

A control, in some embodiments, is the anti-Salmonella antigen antibody titer produced in a subject who has not been administered a Salmonella RNA (e.g., mRNA) vaccine. In some embodiments, a control is an anti-Salmonella antigen antibody titer produced in a subject administered a recombinant or purified Salmonella protein vaccine. Recombinant protein vaccines typically include protein antigens that either have been produced in a heterologous expression system (e.g., bacteria or yeast) or purified from large amounts of the pathogenic organism.

In some embodiments, the ability of a Salmonella vaccine to be effective is measured in a murine model. For example, the Salmonella vaccines may be administered to a murine model and the murine model assayed for induction of neutralizing antibody titers. Pathogen challenge studies may also be used to assess the efficacy of a vaccine of the present disclosure. For example, the Salmonella vaccines may be administered to a murine model, the murine model challenged with Salmonella pathogen, and the murine model assayed for survival and/or immune response (e.g., neutralizing antibody response, T cell response (e.g., cytokine response)).

In some embodiments, an effective amount of a Salmonella RNA (e.g., mRNA) vaccine is a dose that is reduced compared to the standard of care dose of a recombinant Salmonella protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified Salmonella protein vaccine, or a live attenuated or inactivated Salmonella vaccine, or a Salmonella VLP vaccine, that a physician/clinician or other medical professional would administer to a subject to treat or prevent Salmonella, or a Salmonella-related condition, while following the standard of care guideline for treating or preventing Salmonella, or a Salmonella-related condition.

In some embodiments, the anti-Salmonella antigen antibody titer produced in a subject administered an effective amount of a Salmonella RNA vaccine is equivalent to an anti-Salmonella antigen antibody titer produced in a control subject administered a standard of care dose of a recombinant or purified Salmonella protein vaccine, or a live attenuated or inactivated Salmonella vaccine, or a Salmonella VLP vaccine.

In some embodiments, an effective amount of a Salmonella RNA (e.g., mRNA) vaccine is a dose equivalent to an at least 2-fold reduction in a standard of care dose of a recombinant or purified Salmonella protein vaccine. For example, an effective amount of a Salmonella RNA vaccine may be a dose equivalent to an at least 3-fold, at least 4-fold, at least 5-fold, at least 6-fold, at least 7-fold, at least 8-fold, at least 9-fold, or at least 10-fold reduction in a standard of care dose of a recombinant or purified Salmonella protein vaccine. In some embodiments, an effective amount of a Salmonella RNA vaccine is a dose equivalent to an at least at least 100-fold, at least 500-fold, or at least 1000-fold reduction in a standard of care dose of a recombinant or purified Salmonella protein vaccine. In some embodiments, an effective amount of a Salmonella RNA vaccine is a dose equivalent to a 2-, 3-, 4-, 5-, 6-, 7-, 8-, 9-, 10-, 20-, 50-, 100-, 250-, 500-, or 1000-fold reduction in a standard of care dose of a recombinant or purified Salmonella protein vaccine. In some embodiments, the anti-Salmonella antigen antibody titer produced in a subject administered an effective amount of a Salmonella RNA vaccine is equivalent to an anti-Salmonella antigen antibody titer produced in a control subject administered the standard of care dose of a recombinant or protein Salmonella protein vaccine, or a live attenuated or inactivated Salmonella vaccine, or a Salmonella VLP vaccine. In some embodiments, an effective amount of a Salmonella RNA (e.g., mRNA) vaccine is a dose equivalent to a 2-fold to 1000-fold (e.g., 2-fold to 100-fold, 10-fold to 1000-fold) reduction in the standard of care dose of a recombinant or purified Salmonella protein vaccine, wherein the anti-Salmonella antigen antibody titer produced in the subject is equivalent to an anti-Salmonella antigen antibody titer produced in a control subject administered the standard of care dose of a recombinant or purified Salmonella protein vaccine, or a live attenuated or inactivated Salmonella vaccine, or a Salmonella VLP vaccine.

In some embodiments, the effective amount of a Salmonella RNA (e.g., mRNA) vaccine is a dose equivalent to a 2 to 1000-, 2 to 900-, 2 to 800-, 2 to 700-, 2 to 600-, 2 to 500-, 2 to 400-, 2 to 300-, 2 to 200-, 2 to 100-, 2 to 90-, 2 to 80-, 2 to 70-, 2 to 60-, 2 to 50-, 2 to 40-, 2 to 30-, 2 to 20-, 2 to 10-, 2 to 9-, 2 to 8-, 2 to 7-, 2 to 6-, 2 to 5-, 2 to 4-, 2 to 3-, 3 to 1000-, 3 to 900-, 3 to 800-, 3 to 700-, 3 to 600-, 3 to 500-, 3 to 400-, 3 to 3 to 00-, 3 to 200-, 3 to 100-, 3 to 90-, 3 to 80-, 3 to 70-, 3 to 60-, 3 to 50-, 3 to 40-, 3 to 30-, 3 to 20-, 3 to 10-, 3 to 9-, 3 to 8-, 3 to 7-, 3 to 6-, 3 to 5-, 3 to 4-, 4 to 1000-, 4 to 900-, 4 to 800-, 4 to 700-, 4 to 600-, 4 to 500-, 4 to 400-, 4 to 4 to 00-, 4 to 200-, 4 to 100-, 4 to 90-, 4 to 80-, 4 to 70-, 4 to 60-, 4 to 50-, 4 to 40-, 4 to 30-, 4 to 20-, 4 to 10-, 4 to 9-, 4 to 8-, 4 to 7-, 4 to 6-, 4 to 5-, 4 to 4-, 5 to 1000-, 5 to 900-, 5 to 800-, 5 to 700-, 5 to 600-, 5 to 500-, 5 to 400-, 5 to 300-, 5 to 200-, 5 to 100-, 5 to 90-, 5 to 80-, 5 to 70-, 5 to 60-, 5 to 50-, 5 to 40-, 5 to 30-, 5 to 20-, 5 to 10-, 5 to 9-, 5 to 8-, 5 to 7-, 5 to 6-, 6 to 1000-, 6 to 900-, 6 to 800-, 6 to 700-, 6 to 600-, 6 to 500-, 6 to 400-, 6 to 300-, 6 to 200-, 6 to 100-, 6 to 90-, 6 to 80-, 6 to 70-, 6 to 60-, 6 to 50-, 6 to 40-, 6 to 30-, 6 to 20-, 6 to 10-, 6 to 9-, 6 to 8-, 6 to 7-, 7 to 1000-, 7 to 900-, 7 to 800-, 7 to 700-, 7 to 600-, 7 to 500-, 7 to 400-, 7 to 300-, 7 to 200-, 7 to 100-, 7 to 90-, 7 to 80-, 7 to 70-, 7 to 60-, 7 to 50-, 7 to 40-, 7 to 30-, 7 to 20-, 7 to 10-, 7 to 9-, 7 to 8-, 8 to 1000-, 8 to 900-, 8 to 800-, 8 to 700-, 8 to 600-, 8 to 500-, 8 to 400-, 8 to 300-, 8 to 200-, 8 to 100-, 8 to 90-, 8 to 80-, 8 to 70-, 8 to 60-, 8 to 50-, 8 to 40-, 8 to 30-, 8 to 20-, 8 to 10-, 8 to 9-, 9 to 1000-, 9 to 900-, 9 to 800-, 9 to 700-, 9 to 600-, 9 to 500-, 9 to 400-, 9 to 300-, 9 to 200-, 9 to 100-, 9 to 90-, 9 to 80-, 9 to 70-, 9 to 60-, 9 to 50-, 9 to 40-, 9 to 30-, 9 to 20-, 9 to 10-, 10 to 1000-, 10 to 900-, 10 to 800-, 10 to 700-, 10 to 600-, 10 to 500-, 10 to 400-, 10 to 300-, 10 to 200-, 10 to 100-, 10 to 90-, 10 to 80-, 10 to 70-, 10 to 60-, 10 to 50-, 10 to 40-, 10 to 30-, 10 to 20-, 20 to 1000-, 20 to 900-, 20 to 800-, 20 to 700-, 20 to 600-, 20 to 500-, 20 to 400-, 20 to 300-, 20 to 200-, 20 to 100-, 20 to 90-, 20 to 80-, 20 to 70-, 20 to 60-, 20 to 50-, 20 to 40-, 20 to 30-, 30 to 1000-, 30 to 900-, 30 to 800-, 30 to 700-, 30 to 600-, 30 to 500-, 30 to 400-, 30 to 300-, 30 to 200-, 30 to 100-, 30 to 90-, 30 to 80-, 30 to 70-, 30 to 60-, 30 to 50-, 30 to 40-, 40 to 1000-, 40 to 900-, 40 to 800-, 40 to 700-, 40 to 600-, 40 to 500-, 40 to 400-, 40 to 300-, 40 to 200-, 40 to 100-, 40 to 90-, 40 to 80-, 40 to 70-, 40 to 60-, 40 to 50-, 50 to 1000-, 50 to 900-, 50 to 800-, 50 to 700-, 50 to 600-, 50 to 500-, 50 to 400-, 50 to 300-, 50 to 200-, 50 to 100-, 50 to 90-, 50 to 80-, 50 to 70-, 50 to 60-, 60 to 1000-, 60 to 900-, 60 to 800-, 60 to 700-, 60 to 600-, 60 to 500-, 60 to 400-, 60 to 300-, 60 to 200-, 60 to 100-, 60 to 90-, 60 to 80-, 60 to 70-, 70 to 1000-, 70 to 900-, 70 to 800-, 70 to 700-, 70 to 600-, 70 to 500-, 70 to 400-, 70 to 300-, 70 to 200-, 70 to 100-, 70 to 90-, 70 to 80-, 80 to 1000-, 80 to 900-, 80 to 800-, 80 to 700-, 80 to 600-, 80 to 500-, 80 to 400-, 80 to 300-, 80 to 200-, 80 to 100-, 80 to 90-, 90 to 1000-, 90 to 900-, 90 to 800-, 90 to 700-, 90 to 600-, 90 to 500-, 90 to 400-, 90 to 300-, 90 to 200-, 90 to 100-, 100 to 1000-, 100 to 900-, 100 to 800-, 100 to 700-, 100 to 600-, 100 to 500-, 100 to 400-, 100 to 300-, 100 to 200-, 200 to 1000-, 200 to 900-, 200 to 800-, 200 to 700-, 200 to 600-, 200 to 500-, 200 to 400-, 200 to 300-, 300 to 1000-, 300 to 900-, 300 to 800-, 300 to 700-, 300 to 600-, 300 to 500-, 300 to 400-, 400 to 1000-, 400 to 900-, 400 to 800-, 400 to 700-, 400 to 600-, 400 to 500-, 500 to 1000-, 500 to 900-, 500 to 800-, 500 to 700-, 500 to 600-, 600 to 1000-, 600 to 900-, 600 to 800-, 600 to 700-, 700 to 1000-, 700 to 900-, 700 to 800-, 800 to 1000-, 800 to 900-, or 900 to 1000-fold reduction in the standard of care dose of a recombinant Salmonella protein vaccine. In some embodiments, such as the foregoing, the anti-Salmonella antigen antibody titer produced in the subject is equivalent to an anti-Salmonella antigen antibody titer produced in a control subject administered the standard of care dose of a recombinant or purified Salmonella protein vaccine, or a live attenuated or inactivated Salmonella vaccine, or a Salmonella VLP vaccine. In some embodiments, the effective amount is a dose equivalent to (or equivalent to an at least) 2-, 3-, 4-, 5-, 6-, 7-, 8-, 9-, 10-, 20-, 30-, 40-, 50-, 60-, 70-, 80-, 90-, 100-, 110-, 120-, 130-, 140-, 150-, 160-, 170-, 1280-, 190-, 200-, 210-, 220-, 230-, 240-, 250-, 260-, 270-, 280-, 290-, 300-, 310-, 320-, 330-, 340-, 350-, 360-, 370-, 380-, 390-, 400-, 410-, 420-, 430-, 440-, 450-, 4360-, 470-, 480-, 490-, 500-, 510-, 520-, 530-, 540-, 550-, 560-, 5760-, 580-, 590-, 600-, 610-, 620-, 630-, 640-, 650-, 660-, 670-, 680-, 690-, 700-, 710-, 720-, 730-, 740-, 750-, 760-, 770-, 780-, 790-, 800-, 810-, 820-, 830-, 840-, 850-, 860-, 870-, 880-, 890-, 900-, 910-, 920-, 930-, 940-, 950-, 960-, 970-, 980-, 990-, or 1000-fold reduction in the standard of care dose of a recombinant Salmonella protein vaccine. In some embodiments, such as the foregoing, an anti-Salmonella antigen antibody titer produced in the subject is equivalent to an anti-Salmonella antigen antibody titer produced in a control subject administered the standard of care dose of a recombinant or purified Salmonella protein vaccine, or a live attenuated or inactivated Salmonella vaccine, or a Salmonella VLP vaccine.

In some embodiments, the effective amount of a Salmonella RNA (e.g., mRNA) vaccine is a total dose of 50-1000 μg. In some embodiments, the effective amount of a Salmonella RNA (e.g., mRNA) vaccine is a total dose of 50-1000, 50-900, 50-800, 50-700, 50-600, 50-500, 50-400, 50-300, 50-200, 50-100, 50-90, 50-80, 50-70, 50-60, 60-1000, 60-900, 60-800, 60-700, 60-600, 60-500, 60-400, 60-300, 60-200, 60-100, 60-90, 60-80, 60-70, 70-1000, 70-900, 70-800, 70-700, 70-600, 70-500, 70-400, 70-300, 70-200, 70-100, 70-90, 70-80, 80-1000, 80-900, 80-800, 80-700, 80-600, 80-500, 80-400, 80-300, 80-200, 80-100, 80-90, 90-1000, 90-900, 90-800, 90-700, 90-600, 90-500, 90-400, 90-300, 90-200, 90-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-900, 600-700, 700-1000, 700-900, 700-800, 800-1000, 800-900, or 900-1000 μg. In some embodiments, the effective amount of a Salmonella RNA (e.g., mRNA) vaccine is a total dose of 50, 100, 150, 200, 250, 300, 350, 400, 450, 500, 550, 600, 650, 700, 750, 800, 850, 900, 950 or 1000 μg. In some embodiments, the effective amount is a dose of 25-500 μg administered to the subject a total of two times. In some embodiments, the effective amount of a Salmonella RNA (e.g., mRNA) vaccine is a dose of 25-500, 25-400, 25-300, 25-200, 25-100, 25-50, 50-500, 50-400, 50-300, 50-200, 50-100, 100-500, 100-400, 100-300, 100-200, 150-500, 150-400, 150-300, 150-200, 200-500, 200-400, 200-300, 250-500, 250-400, 250-300, 300-500, 300-400, 350-500, 350-400, 400-500 or 450-500 μg administered to the subject a total of two times. In some embodiments, the effective amount of a Salmonella RNA (e.g., mRNA) vaccine is a total dose of 25, 50, 100, 150, 200, 250, 300, 350, 400, 450, or 500 μg administered to the subject a total of two times.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and

Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In some embodiments, efficacy of the Salmonella vaccine is at least 60% relative to unvaccinated control subjects. For example, efficacy of the Salmonella vaccine may be at least 65%, at least 70%, at least 75%, at least 80%, at least 85%, at least 95%, at least 98%, or 100% relative to unvaccinated control subjects.

Sterilizing Immunity.

Sterilizing immunity refers to a unique immune status that prevents effective pathogen infection into the host. In some embodiments, the effective amount of a Salmonella vaccine of the present disclosure is sufficient to provide sterilizing immunity in the subject for at least 1 year. For example, the effective amount of a Salmonella vaccine of the present disclosure is sufficient to provide sterilizing immunity in the subject for at least 2 years, at least 3 years, at least 4 years, or at least 5 years. In some embodiments, the effective amount of a Salmonella vaccine of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen.

In some embodiments, the effective amount of a Salmonella vaccine of the present disclosure is sufficient to produce detectable levels of Salmonella antigen as measured in serum of the subject at 1-72 hours post administration.

Titer.

An antibody titer is a measurement of the amount of antibodies within a subject, for example, antibodies that are specific to a particular antigen (e.g., an anti-Salmonella antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of a Salmonella vaccine of the present disclosure is sufficient to produce a 1,000-10,000 neutralizing antibody titer produced by neutralizing antibody against the Salmonella antigen as measured in serum of the subject at 1-72 hours post administration. In some embodiments, the effective amount is sufficient to produce a 1,000-5,000 neutralizing antibody titer produced by neutralizing antibody against the Salmonella antigen as measured in serum of the subject at 1-72 hours post administration. In some embodiments, the effective amount is sufficient to produce a 5,000-10,000 neutralizing antibody titer produced by neutralizing antibody against the Salmonella antigen as measured in serum of the subject at 1-72 hours post administration.

In some embodiments, the neutralizing antibody titer is at least 100 neutralizing units per milliliter (U/ml). For example, the neutralizing antibody titer may be at least 200, 300, 400, 500, 600, 700, 800, 900 or 1000 U/ml. In some embodiments, the neutralizing antibody titer is at least 10,000 U/ml.

In some embodiments, an anti-Salmonella antigen antibody titer produced in the subject is increased by at least 1 log relative to a control. For example, an anti-Salmonella antigen antibody titer produced in the subject may be increased by at least 2, 3, 4, 5, 6, 7, 8, 9 or 10 log relative to a control.

In some embodiments, an anti-Salmonella antigen antibody titer produced in the subject is increased at least 2 times relative to a control. For example, an anti-Salmonella antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a live attenuated Salmonella vaccine, an inactivated Salmonella vaccine, or a protein subunit Salmonella vaccine.

Neutralization Assays.

In some embodiments, the ability of antibodies induced by an antigen of the disclosure to neutralize Salmonella pathogens is measured. For example, in one embodiment, a serum bactericidal antibody (SBA) assay that measures complement mediated killing via antibody can be used. This assay uses active complement, either intrinsic from the serum being tested or the addition of exogenous complement, either from a human or from another species such as rabbit. Antibodies that are capable of opsonizing the bacteria facilitate binding of complement and killing of the bacteria. Alternatively, the ability of an antibody to opsonize bacteria and facilitate uptake by phagocytic cells may also be measured. It will be understood that either of these assays, in addition to measuring neutralization/bactericidal ability of an antibody, may be used to measure functional antibody titers against bacterial pathogens.

### EXAMPLES

### Example 1: Antigen Expression Studies

These studies were designed to test the in vitro expression of Salmonella antigens from various mRNA vaccines of the present disclosure. mRNA vaccines encoding SseB, Mit14, OmpL, OmpC, OmpD, OmpF, IroN, CirA, FepA, T0937, FliC, PiIL, PltB, PltA, CdtB, SlyB, Sty1086, or STY0796 antigens linked to C-terminal His tags were tested. The mRNA constructs were transfected into HEK293F cells. Twenty hours post transfection, the cell culture supernatant (normal or concentrated) or HEK293F cell lysates were collected, and expression of the antigens present in the supernatant or lysate was analyzed by Western blot. Mouse anti-His antibodies were used as the primary antibody, and anti-mouse A1647 antibodies were used as the secondary antibody. GFP and untransfected cells were used as controls. The results of the Western blot are provided below in Tables 1-8. Antigen expression in the supernatant is indicative of antigen secretion from the cells. NGM=non-glycosylation mutant; nIgK=N-terminal humanized IgK signal sequence; nFLRT2=N-terminal humanized FLRT2 signal sequence; cHis=C-terminal His tag (6×).

TABLE 1
Protein Expression of OmpC, SseB, and FliC

| | | | | | WB | |
|---|---|---|---|---|---|---|
| | | | QC | WB | Observed | WB |
| | | Expected | Measured | Observed | MW | Observed |
| | | Molecular | Molecular | MW | Concentrated | MW |
| SEQ ID | | Weight | Weight | Lysate | supernatant | Supernatant |
| NO: | mRNA Name | (kD) | (kD) | (kD) | (kD) | (kD) |
| 20 | St_OmpC_NGM_nIgk_cHis | 42.118 | 43.098 | 42-49* | — | — |
| 24 | St_SseB_cHis | 29.71 | 32.723 | 28-38** | 28-38* | — |
| 28 | St_SseB_nIgK_cHis | 31.833 | 34.804 | 28-38*** | 28-38*** | 28-38* |
| 32 | St_FliC_cHis | 54.093 | 58.599 | 49-62** | 49-62* | — |
| 36 | St_FliC_nIgK_cHis | 56.216 | 60.036 | — | 62** | — |
| — | GFP | — | — | — | — | — |

Relative expression level: *low, **medium, ***high

Table 1 shows results from a Western blot analysis of protein collected from HEK293F cells transfected with:

mRNA encoding an OmpC non-glycosylation mutant (NGM) antigen from Salmonella typhi (St) having a humanized IgK signal sequence and a 6×His tag (St_OmpC_NGM_nIgk_cHis);

mRNA encoding SseB antigen from S. typhi having a 6×His tag (St_SseB cHis);

mRNA encoding a SseB antigen from S. typhi having a humanized IgK signal sequence and a 6×His tag (St_SseB_nIgk_cHis);

mRNA encoding a FliC antigen from S. typhi having a 6×His tag (St_FliC_cHis);

mRNA encoding a FliC antigen from S. typhi having a humanized IgK signal sequence and a 6×His tag (St_OmpC_NGM_nIgk_cHis); or mRNA encoding GFP as a control.

TABLE 2
Protein Expression of OmpC and Mig14

| | | | | | WB | |
|---|---|---|---|---|---|---|
| | | | QC | WB | Observed | WB |
| | | Expected | Measured | Observed | MW | Observed |
| SEQ | | Molecular | Molecular | MW | Concentrated | MW |
| ID | | Weight | Weight | Lysate | supernatant | Supernatant |
| NO: | mRNA Name | (kD) | (kD) | (kD) | (kD) | (kD) |
| 20 | St_OmpC_NGM_nIgk_cHis | 42.118 | 43.098 | 42 & 44* | — | — |
| 2 | St_OmpC_nIgK_cHis | 42.256 | 42.593 | 62* | — | — |
| 58 | St_Mig14_cHis | 36.009 | 33.634 | — | — | — |
| 62 | St_Mig14_nIgK_cHis | 38.098 | 34.873 | 38*** | — | — |
| 66 | St_Mig14_NGM_cHis | 35.977 | 33.386 | — | — | — |
| 70 | St_Mig14_NGM_nIgK_cHis | 38.066 | 34.109 | 38*** | — | — |
| — | GFP | — | — | — | — | — |

Relative expression level: *low, **medium, ***high

Table 2 shows results from a Western blot analysis of protein collected from HEK293F cells transfected with:

mRNA encoding an OmpC NGM antigen from S. typhi having a humanized IgK signal sequence and a 6×His tag (St_OmpC_NGM_nIgk_cHis);

mRNA encoding OmpC antigen from S. typhi having a humanized IgK signal sequence and a 6×His tag (St_Omp_nIgK_cHis);

mRNA encoding a Mig14 antigen from S. typhi having a 6×His tag (St_Mig14_cHis);

mRNA encoding a Mig14 antigen from S. typhi having a humanized IgK signal sequence and a 6×His tag (St_Mig14_nIgK_cHis);

mRNA encoding a Mig14_NGM antigen from S. typhi having a 6×His tag (St_Mig14_NGM_cHis);

mRNA encoding a Mig14_NGM antigen from S. typhi having a humanized IgK signal sequence and a 6×His tag (St_Mig14_NGM_cHis); or mRNA encoding GFP as a control.

TABLE 3
Protein Expression of OmpF, OmpL, and CirA

| | | | | | WB | |
|---|---|---|---|---|---|---|
| | | | QC | WB | Observed | WB |
| | | Expected | Measured | Observed | MW | Observed |
| SEQ | | Molecular | Molecular | MW | Concentrated | MW |
| ID | | Weight | Weight | Lysate | supernatant | Supernatant |
| NO: | mRNA Name | (kD) | (kD) | (kD) | (kD) | (kD) |
| 8 | St_OmpF_nIgK_cHis variant | 40.963 | 41.724 | — | — | — |
| 106 | St_OmpL_nIgK_cHis | 28.106 | 27.381 | 38** | — | — |
| 110 | St_OmpL_NGM_nIgK_cHis | 28.03 | 27.599 | 28* | — | — |
| 80 | St_CirA_nFLRT2_cHis | 75.618 | 77.01 | — | — | — |
| 84 | St_CirA_NGM_nFLRT2_cHis | 75.498 | 79.063 | 70-98** | 70-98** | — |
| — | GFP | — | — | — | — | — |

Relative expression level: *low, **medium, ***high

Table 3 shows results from a Western blot analysis of protein collected from HEK293F cells transfected with:

mRNA encoding an OmpF antigen variant from S. typhi having a humanized IgK signal sequence and a 6×His tag (St_OmpF_nIgk_cHis);

mRNA encoding an OmpL antigen from S. typhi having a humanized IgK signal sequence and a 6×His tag (St_OmpL_nIgk_cHis);

mRNA encoding an OmpL NGM antigen from S. typhi having a humanized IgK signal sequence and a 6×His tag (St_OmpL_NGM_nIgk_cHis);

mRNA encoding a CirA antigen from S. typhi having a humanized FLRT2 signal sequence and a 6×His tag (St_CirA_nFLRT2_cHis);

mRNA encoding a CirA NGM antigen from S. typhi having a humanized FLRT2 signal sequence and a 6×His tag (St_CirA_NGM_nFLRT2_cHis); or mRNA encoding GFP as a control.

TABLE 4
Protein Expression of Typhoid Toxin Subunits

| | | | | | Observed |
|---|---|---|---|---|---|
| | | Expected | QC | Observed | MW |
| | | MW | Measured | MW Lysate | Sup/cSup |
| SEQ ID NO: | mRNA Construct | (kD) | MW (kD) | (kD) | (kD) |
| 102 | St_CdtB_NGM_nIgK_cHis | 30.112 | 32.686 | 30** | 30* cS |
| 158 | St_CdtB_nIgK_cHis | 30.128 | 31.767 | 30** | 30* cS |
| 118 | St_PltA_NGM_nIgK_cHis | 28.142 | 27.909 | 35* | ~ |
| 114 | St_PltA_nIgK_cHis | 28.172 | 28.954 | 38* | ~ |
| 126 | St_PltB_NGM_nIgK_cHis | 15.615 | 15.507 | 14** | ~ |
| 122 | St_PltB_nIgK_cHis | 16.631 | 16.481 | 14** | ~ |

Relative expression level:
*low,
**medium,
***high

Table 4 shows results from a Western blot analysis of protein collected from HEK293F cells transfected with:

mRNA encoding CdtB NGM antigen from S. typhi having a humanized IgK signal sequence and a 6×His tag (St_CdtB_NGM_nIgK_cHis);

mRNA encoding CdtB antigen from S. typhi having a humanized IgK signal sequence and a 6×His tag (St_CdtB_nIgK_cHis);

mRNA encoding PltA NGM antigen from S. typhi having a humanized IgK signal sequence and a 6×His tag (St_PltA_NGM_nIgK_cHis);

mRNA encoding PltA antigen from S. typhi having a humanized IgK signal sequence and a 6×His tag (St_PltA_nIgK_cHis);

mRNA encoding PtlB NGM antigen from S. typhi having a humanized IgK signal sequence and a 6×His tag (St_PtlB_NGM_nIgK_cHis); or

mRNA encoding PtlB antigen from S. typhi having a humanized IgK signal sequence and a 6×His tag (St_PtlB_nIgK_cHis).

TABLE 5
Protein Expression of FepA, OmpF, and OmpD

| | | | | | WB | |
|---|---|---|---|---|---|---|
| | | | QC | WB | Observed | WB |
| | | Expected | Measured | Observed | MW | Observed |
| | | Molecular | Molecular | MW | Concentrated | MW |
| SEQ ID | | Weight | Weight | Lysate | supernatant | Supernatant |
| NO: | mRNA Name | (kD) | (kD) | (kD) | (kD) | (kD) |
| 90 | St_FepA_nIgK_cHis | 83.439 | 90.33 | 95-70** | — | — |
| 94 | St_FepA_NGM_nIgK_cHis | 83.227 | 88.344 | — | — | — |
| 12 | St_OmpF_NGM_nIgK_cHis | 40.917 | 41.793 | 40* | — | — |
| | variant | | | | | |
| 16 | Stm_OmpD_nIgK_cHis | 40.707 | 43.323 | — | — | — |
| | variant | | | | | |
| — | GFP | — | — | — | — | — |

Relative expression level: *low, **medium, ***high

Table 5 shows results from a Western blot analysis of protein collected from HEK293F cells transfected with:

mRNA encoding an FepA antigen from S. typhi having a humanized IgK signal sequence and a 6×His tag (St_FepA_nIgk_cHis);

mRNA encoding an FepA NGM antigen from S. typhi having a humanized IgK signal sequence and a 6×His tag (St_FepA_NGM_nIgk_cHis);

mRNA encoding an OmpF NGM antigen from S. typhi having a humanized IgK signal sequence and a 6×His tag (St_OmpF_NGM_nIgk_cHis);

mRNA encoding an OmpD antigen variant from S. typhimurium (Stm) having a humanized IgK signal sequence and a 6×His tag (Stm_OmpD_nIgk_cHis); or mRNA encoding GFP as a control.

TABLE 6
Protein Expression of FliC variants

| | | Expected | QC measured | |
|---|---|---|---|---|
| SEQ | | Molecular | Molecular | Observed |
| ID | | Weight | Weight | MW cSup |
| NO: | mRNA name | (kD) | (kD) | (kD) |
| 44 | SpA_FliC_nIgK_cHis | 54.84 | 57.203 | ~62 |
| 48 | Stm_FliC_cHis | 52.434 | 56.325 | ~51 |
| 52 | Stm_FliC_nIgK_cHis | 54.5 | 54.962 | ~62 |
| — | GFP | 26.941 | 27.313 | — |

Relative expression level:
*low,
**medium,
***high

Table 6 shows results from a Western blot analysis of protein collected from HEK293F cells transfected with:

mRNA encoding an FliC antigen from S. paratphi (SpA) having a humanized IgK signal sequence and a 6×His tag (SpA_FliC_nIgk_cHis);

mRNA encoding an FliC antigen from S. typhimurium (Stm) having a 6×His tag (Stm_FliC_cHis);

mRNA encoding an FliC antigen from S. typhimurium (Stm) having a humanized IgK signal sequence and a 6×His tag (Stm_FliC_cHis); or

mRNA encoding GFP as a control.

TABLE 7
Protein Expression of IroN and ViMimotope

| | | | | | WB | |
|---|---|---|---|---|---|---|
| | | | QC | WB | Observed | WB |
| | | Expected | Measured | Observed | MW | Observed |
| | | Molecular | Molecular | MW | Concentrated | MW |
| SEQ ID | | Weight | Weight | Lysate | supernatant | Supernatant |
| NO: | mRNA Name | (kD) | (kD) | (kD) | (kD) | (kD) |
| 72 | St_IroN_nFLRT2_cHis | 81.32 | 90.932 | 75-98** | — | — |
| 88 | St_ViMimo_Lumazine_cHis | 24.206 | 23.213 | — | — | — |
| — | GFP | — | — | — | — | — |

Relative expression level: *low, ** medium, ***high

Table 7 shows results from a Western blot analysis of protein collected from HEK293F cells transfected with:

mRNA encoding an IroN antigen from S. typhi having a humanized FLRT2 signal sequence and a 6×His tag (St_IroN_nFLRT2_cHis);

mRNA encoding a ViMimotope antigen (a polysaccharide that mimics the Vi Salmonella construct) fused to lumazine from S. typhi having a 6×His tag (St_ViMimo_Lumazine_cHis); or

mRNA encoding GFP as a control.

While the ViMimotope was not detected by Western blot, it was detected by liquid chromatography-mass spectrometry (LCMS) (data not shown).

TABLE 8
Protein Expression of T0937 and SlyB

| | | | | | WB | |
|---|---|---|---|---|---|---|
| | | | QC | WB | Observed | WB |
| | | Expected | Measured | Observed | MW | Observed |
| SEQ | | Molecular | Molecular | MW | Concentrated | MW |
| ID | | Weight | Weight | Lysate | supernatant | Supernatant |
| NO: | mRNA Name | (kD) | (kD) | (kD) | (kD) | (kD) |
| 133 | St_T0937_nIgk_NGM_cHis | 55 | 56 | 55** | 55** | — |
| 135 | St_T0937_nIgk_nTrunc_NGM_cHis | 51 | 47 | 2 bands: | — | — |
| | | | | 47**, | | |
| | | | | 45** | | |
| 137 | St_slyB_nIgk_NGM_cHis | 16 | 17 | 15** | 17** | 17* |
| — | GFP | — | — | — | — | — |

Relative expression level: *low, **medium, ***high

Table 8 shows results from a Western blot analysis of protein collected from HEK293F cells transfected with:

mRNA encoding an T0937 NGM antigen from S. typhi having a humanized IgK signal sequence and a 6×His tag (St_T0937_nIgK_NGM_cHis);

mRNA encoding an a truncated T0937 NGM antigen from S. typhi having a humanized IgK signal sequence and a 6×His tag (St_T0937_nIgK_nTrunc_NGM_cHis);

mRNA encoding an SlyB NGM antigen from S. typhi having a humanized IgK signal sequence and a 6×His tag (St_slyB_nIgK_NGM_cHis); or

mRNA encoding GFP as a control.

TABLE 9
Protein Expression of STY0796 and STY1086

| | | | | | WB | |
|---|---|---|---|---|---|---|
| | | | QC | WB | Observed | WB |
| | | Expected | Measured | Observed | MW | Observed |
| SEQ | | Molecular | Molecular | MW | Concentrated | MW |
| ID | | Weight | Weight | Lysate | supernatant | Supernatant |
| NO: | mRNA Name | (kD) | (kD) | (kD) | (kD) | (kD) |
| 144 | St_STY0796_nIgK_cHis | 29 | 30 | — | 40** | — |
| 146 | St_STY0796_NGM_nIgK_cHis | 28 | 31 | — | 35** | 35** |
| 148 | St_STY1086_nIgK_cHis | 21 | 22 | — | 2 bands: | — |
| | | | | | 30**, 22** | |
| 150 | St_STY1086_NGM_nIgK_cHis | 21 | 22 | 1788 | 2 bands: | — |
| | | | | | 22**, 17** | |

Relative expression level: *low, **medium, ***high

Table 9 shows results from a Western blot analysis of protein collected from HEK293F cells transfected with:

mRNA encoding a STY0796 antigen from S. typhi having a humanized IgK signal sequence and a 6×His tag (St_STY0796_nIgK_cHis);

mRNA encoding a STY0796 NGM antigen from S. typhi having a humanized IgK signal sequence and a 6×His tag (St_STY0796_NGM_nIgK_cHis);

mRNA encoding a STY1086 antigen from S. typhi having a humanized IgK signal sequence and a 6×His tag (St_STY0796_nIgK_cHis);

mRNA encoding a STY1086 NGM antigen from S. typhi having a humanized IgK signal sequence and a 6×His tag (St_STY0796_NGM_nIgK_cHis); or mRNA encoding GFP as a control.

### Example 2: Salmonella Immunogenicity Studies

The instant study was designed to test the immunogenicity of various Salmonella mRNA vaccines formulated in a lipid nanoparticle in vivo. Female BALB/c mice, 6-8 weeks of age (n=5 per group), were vaccinated intramuscularly with Salmonella mRNA vaccine encoding SseB (construct St_SseB_nIgK; SEQ ID NO:30 (mRNA ORF); SEQ ID NO:29 (protein ORF)) formulated in a lipid nanoparticle comprising Formula (I), Compound 1 lipids at a concentration of 0.2 mg/ml (10 μg SseB) or 0.04 mg/ml (2 μg SseB) on day 1. The mice were given a booster dose on day 29, and spleens were harvested on day 43. Blood samples were drawn three days before the first immunization, and then again on day 28, day 36, and day 43. Serum was isolated and stored at 20° C. On day 36, half of the mice were euthanized for spleen collection. Two groups were given PBS as a control (half were euthanized on day 36, and the other half were sacrificed on day 43).

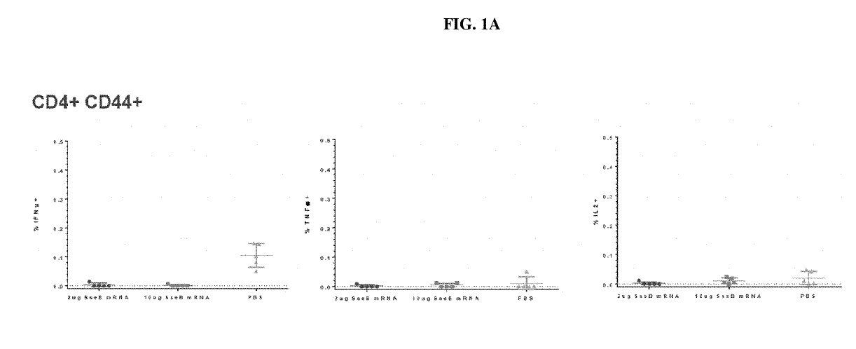

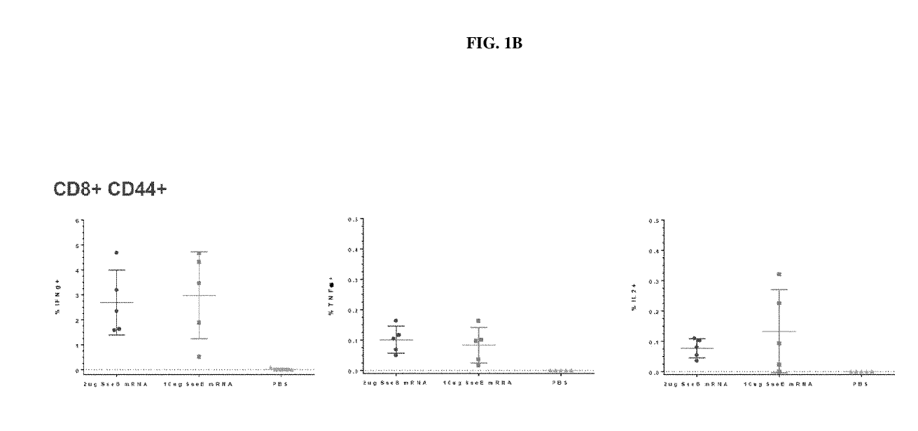

As shown in FIGS. 1A-1B, the Salmonella mRNA vaccine encoding SseB (construct St_SseB_nIgK; SEQ ID NO:30 (mRNA ORF); SEQ ID NO:29 (protein ORF)) was found to elicit SseB specific, IFN-γ, TNF-α and IL-2 secreting CD8⁺ T cells.

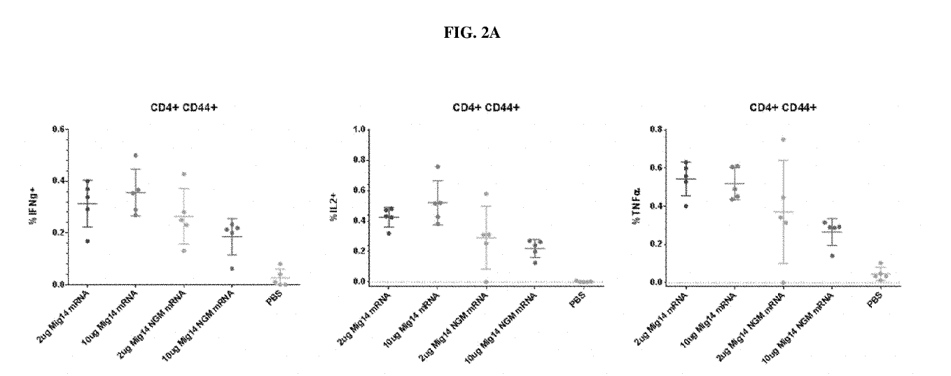

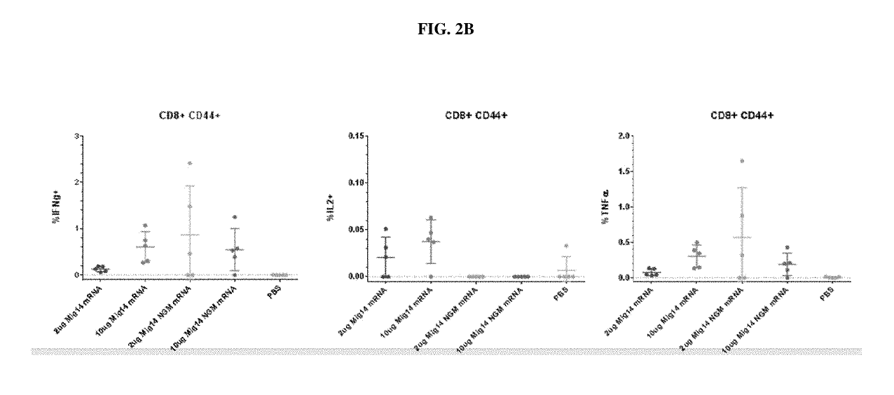

The study was repeated using Mig14 with (construct St_Mig14_NGM_nIgK; SEQ ID NO:68 (mRNA ORF); SEQ ID NO:67 (protein ORF)) and without (construct St_Mig14_nIgK; SEQ ID NO:59 (mRNA ORF); SEQ ID NO:60 (protein ORF)) mutations at predicted N-linked glycosylation sites) mRNA. As shown in FIGS. 2A-2B, both versions of Mig14 mRNA were able to elicit both CD4⁺ and CD8⁺ T cells.

### EQUIVALENTS

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

The entire contents of International Application Nos. PCT/US2015/02740, PCT/US2016/043348, PCT/US2016/043332, PCT/US2016/058327, PCT/US2016/058324, PCT/US2016/058314, PCT/US2016/058310, PCT/US2016/058321, PCT/US2016/058297, PCT/US2016/058319, and PCT/US2016/058314 are incorporated herein by reference.

### SEQUENCE LISTING

It should be understood that any of the mRNA sequences described herein may include a 5′ UTR and/or a 3′ UTR. The UTR sequences may be selected from the following sequences, or other known UTR sequences may be used. It should also be understood that any of the mRNA constructs described herein may further comprise a polyA tail and/or cap (e.g., 7mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
GGGAAAUAAGAGAGAAAAGAAGAGUAAGAAGAAAUAUAAGAGCCACC
(SEQ ID NO: 3)
5′ UTR:
GGGAAAUAAGAGAGAAAAGAAGAGUAAGAAGAAAUAUAAGACCCCGGCGCCGCCACC
(SEQ ID NO: 140)
3′ UTR:
UGAUAAUAGGCUGGAGCCUCGGUGGCCAUGCUUCUUGCCCCUUGGGCCUCCCCCCAGCCCCUCC
UCCCCUUCCUGCACCCGUACCCCCGUGGUCUUUGAAUAAAGUCUGAGUGGGCGGC
(SEQ ID NO: 4)
3′ UTR:
UGAUAAUAGGCUGGAGCCUCGGUGGCCUAGCUUCUUGCCCCUUGGGCCUCCCCCCAGCCCCUCC
UCCCCUUCCUGCACCCGUACCCCCGUGGUCUUUGAAUAAAGUCUGAGUGGGCGGC
(SEQ ID NO: 129)

| | | | | 5 UTR | |
|---|---|---|---|---|---|
| | mRNA | Orf Sequence | Orf Sequence | Se- | 3 UTR |
| | Name | (Amino Acid) | (Nucleotide) | quence | Sequence |
| SEQ ID NO: | | 1 | 2 | 3 | 4 |

SEQ ID NO: 161 consists of from 5′ end to 3′ end, 5′ UTR SEQ
ID NO: 3, mRNA ORF SEQ ID NO: 2, and 3′ UTR SEQ ID NO: 4.

| | St_OmpC_ | METPAQLLFLLLLWL | AUGGAAACCCCUGC | GGGAAA | UGAUAA |
|---|---|---|---|---|---|
| | nIgK_cHis | PDTTGAEIYNKDGNK | UCAGCUGCUGUUCC | UAAGAG | UAGGCU |
| | IgK | LDLFGKVDGLHYFSD | UGCUGCUGCUGUGG | AGAAAA | GGAGCC |
| | underlined: | DKGSDGDQTYMRIG | CUGCCUGAUACAAC | GAAGAG | UCGGUG |
| | METPAQ | FKGETQVNDQLTGY | AGGCGCCGAGAUCU | UAAGAA | GCCAUG |
| | LLFLLLL | GQWEYQIQGNQTEG | ACAACAAGGACGGC | GAAAUA | CUUCUU |
| | WLPDTT | SNDSWTRVAFAGLK | AACAAGCUGGACCU | UAAGAG | GCCCCU |
| | G (SEQ ID | FADAGSFDYGRNYG | GUUCGGCAAGGUGG | CCACC | UGGGCC |
| | NO: 153) | VTYDVTSWTDVLPEF | ACGGCCUGCACUAC | | UCCCCC |
| | AUGGAA | GGDTYGADNFMQQR | UUCAGCGACGAUAA | | CAGCCC |
| | ACCCCU | GNGYATYRNTDFFG | GGGCUCCGACGGCG | | CUCCUC |
| | GCUCAG | LVDGLDFALQYQGK | ACCAGACCUACAUG | | CCCUUC |
| | CUGCUG | NGSVSGENTNGRSLL | AGAAUCGGCUUCAA | | CUGCAC |
| | UUCCUG | NQNGDGYGGSLTYAI | GGGCGAGACACAAG | | CCGUAC |
| | CUGCUG | GEGFSVGGAITTSKR | UGAACGACCAGCUG | | CCCCGU |
| | CUGUGG | TADQNNTANARLYG | ACAGGCUACGGCCA | | GGUCUU |
| | CUGCCU | NGDRATVYTGGLKY | GUGGGAGUAUCAGA | | UGAAUA |
| | GAUACA | DANNIYLAAQYSQTY | UCCAGGGCAAUCAG | | AAGUCU |
| | ACAGGC | NATRFGTSNGSNPST | ACCGAGGGCAGCAA | | GAGUGG |
| | (SEQ ID | SYGFANKAQNFEVV | CGACAGCUGGACCA | | GCGGC |
| | NO: 235) | AQYQFDFGLRPSVAY | GAGUGGCUUUUGCC | | |
| | | LQSKGKDISNGYGAS | GGCCUGAAGUUUGC | | |
| | | YGDQDIVKYVDVGA | CGAUGCCGGCAGCU | | |
| | | TYYFNKNMSTYVDY | UUGACUACGGCAGA | | |
| | | KINLLDKNDFTRDAG | AAUUACGGCGUGAC | | |
| | | INTDDIVALGLVYQF | CUACGACGUGACCU | | |
| | | HHHHHH | CCUGGACAGAUGUG | | |
| | | | CUGCCUGAGUUUGG | | |
| | | | CGGCGAUACCUACG | | |
| | | | GCGCCGACAACUUC | | |
| | | | AUGCAGCAGAGAGG | | |
| | | | CAACGGCUACGCCA | | |
| | | | CCUACCGGAACACC | | |
| | | | GAUUUCUUCGGCCU | | |
| | | | GGUGGAUGGCCUGG | | |
| | | | AUUUCGCCCUGCAG | | |
| | | | UACCAGGGCAAGAA | | |
| | | | UGGCUCUGUGUCCG | | |
| | | | GCGAGAACACCAAC | | |
| | | | GGCAGAAGCCUGCU | | |
| | | | GAACCAGAACGGCG | | |
| | | | ACGGAUAUGGCGGC | | |
| | | | AGCCUGACAUAUGC | | |
| | | | CAUCGGCGAGGGCU | | |
| | | | UUUCUGUCGGCGGA | | |
| | | | GCCAUCACCACCAG | | |
| | | | CAAGAGAACAGCCG | | |
| | | | ACCAGAACAACACC | | |
| | | | GCCAACGCCAGACU | | |
| | | | GUACGGCAACGGCG | | |
| | | | AUAGAGCCACAGUG | | |
| | | | UAUACCGGCGGACU | | |
| | | | GAAGUACGACGCCA | | |
| | | | ACAACAUCUACCUG | | |
| | | | GCCGCUCAGUACAG | | |
| | | | CCAGACAUACAACG | | |
| | | | CCACCAGAUUCGGC | | |
| | | | ACCAGCAACGGCAG | | |
| | | | CAAUCCCAGCACAA | | |
| | | | GCUACGGCUUCGCC | | |
| | | | AACAAGGCCCAGAA | | |
| | | | CUUUGAGGUGGUGG | | |
| | | | CCCAGUACCAGUUC | | |
| | | | GACUUCGGACUGAG | | |
| | | | GCCUAGCGUGGCCU | | |
| | | | ACCUGCAGAGCAAG | | |
| | | | GGCAAAGACAUCAG | | |
| | | | CAACGGAUACGGCG | | |
| | | | CCAGCUACGGCGAU | | |
| | | | CAGGACAUCGUGAA | | |
| | | | AUACGUGGACGUGG | | |
| | | | GCGCCACCUAUUAC | | |
| | | | UUCAACAAGAACAU | | |
| | | | GAGCACCUACGUGG | | |
| | | | ACUACAAGAUCAAC | | |
| | | | CUGCUGGACAAGAA | | |
| | | | CGACUUCACCCGCG | | |
| | | | ACGCCGGCAUCAAC | | |
| | | | ACCGAUGAUAUUGU | | |
| | | | GGCCCUGGGCCUCG | | |
| | | | UGUACCAGUUUCAC | | |
| | | | CACCACCAUCACCA | | |
| | | | U | | |
| SEQ ID NO: | | 5 | 6 | 3 | 4 |

SEQ ID NO: 162 consists of from 5′ end to 3′ end, 5′ UTR SEQ
ID NO: 3, mRNA ORF SEQ ID NO: 6, and 3′ UTR SEQ ID NO: 4.

| | St_OmpC_ | METPAQLLFLLLLWL | AUGGAAACCCCUGC | GGGAAA | UGAUAA |
|---|---|---|---|---|---|
| | nIgK | PDTTGAEIYNKDGNK | UCAGCUGCUGUUCC | UAAGAG | UAGGCU |
| | | LDLFGKVDGLHYFSD | UGCUGCUGCUGUGG | AGAAAA | GGAGCC |
| | | DKGSDGDQTYMRIG | CUGCCUGAUACAAC | GAAGAG | UCGGUG |
| | | FKGETQVNDQLTGY | AGGCGCCGAGAUCU | UAAGAA | GCCAUG |
| | | GQWEYQIQGNQTEG | ACAACAAGGACGGC | GAAAUA | CUUCUU |
| | | SNDSWTRVAFAGLK | AACAAGCUGGACCU | UAAGAG | GCCCCU |
| | | FADAGSFDYGRNYG | GUUCGGCAAGGUGG | CCACC | UGGGCC |
| | | VTYDVTSWTDVLPEF | ACGGCCUGCACUAC | | UCCCCC |
| | | GGDTYGADNFMQQR | UUCAGCGACGAUAA | | CAGCCC |
| | | GNGYATYRNTDFFG | GGGCUCCGACGGCG | | CUCCUC |
| | | LVDGLDFALQYQGK | ACCAGACCUACAUG | | CCCUUC |
| | | NGSVSGENTNGRSLL | AGAAUCGGCUUCAA | | CUGCAC |
| | | NQNGDGYGGSLTYAI | GGGCGAGACACAAG | | CCGUAC |
| | | GEGFSVGGAITTSKR | UGAACGACCAGCUG | | CCCCGU |
| | | TADQNNTANARLYG | ACAGGCUACGGCCA | | GGUCUU |
| | | NGDRATVYTGGLKY | GUGGGAGUAUCAGA | | UGAAUA |
| | | DANNIYLAAQYSQTY | UCCAGGGCAAUCAG | | AAGUCU |
| | | NATRFGTSNGSNPST | ACCGAGGGCAGCAA | | GAGUGG |
| | | SYGFANKAQNFEVV | CGACAGCUGGACCA | | GCGGC |
| | | AQYQFDFGLRPSVAY | GAGUGGCUUUUGCC | | |
| | | LQSKGKDISNGYGAS | GGCCUGAAGUUUGC | | |
| | | YGDQDIVKYVDVGA | CGAUGCCGGCAGCU | | |
| | | TYYFNKNMSTYVDY | UUGACUACGGCAGA | | |
| | | KINLLDKNDFTRDAG | AAUUACGGCGUGAC | | |
| | | INTDDIVALGLVYQF | CUACGACGUGACCU | | |
| | | | CCUGGACAGAUGUG | | |
| | | | CUGCCUGAGUUUGG | | |
| | | | CGGCGAUACCUACG | | |
| | | | GCGCCGACAACUUC | | |
| | | | AUGCAGCAGAGAGG | | |
| | | | CAACGGCUACGCCA | | |
| | | | CCUACCGGAACACC | | |
| | | | GAUUUCUUCGGCCU | | |
| | | | GGUGGAUGGCCUGG | | |
| | | | AUUUCGCCCUGCAG | | |
| | | | UACCAGGGCAAGAA | | |
| | | | UGGCUCUGUGUCCG | | |
| | | | GCGAGAACACCAAC | | |
| | | | GGCAGAAGCCUGCU | | |
| | | | GAACCAGAACGGCG | | |
| | | | ACGGAUAUGGCGGC | | |
| | | | AGCCUGACAUAUGC | | |
| | | | CAUCGGCGAGGGCU | | |
| | | | UUUCUGUCGGCGGA | | |
| | | | GCCAUCACCACCAG | | |
| | | | CAAGAGAACAGCCG | | |
| | | | ACCAGAACAACACC | | |
| | | | GCCAACGCCAGACU | | |
| | | | GUACGGCAACGGCG | | |
| | | | AUAGAGCCACAGUG | | |
| | | | UAUACCGGCGGACU | | |
| | | | GAAGUACGACGCCA | | |
| | | | ACAACAUCUACCUG | | |
| | | | GCCGCUCAGUACAG | | |
| | | | CCAGACAUACAACG | | |
| | | | CCACCAGAUUCGGC | | |
| | | | ACCAGCAACGGCAG | | |
| | | | CAAUCCCAGCACAA | | |
| | | | GCUACGGCUUCGCC | | |
| | | | AACAAGGCCCAGAA | | |
| | | | CUUUGAGGUGGUGG | | |
| | | | CCCAGUACCAGUUC | | |
| | | | GACUUCGGACUGAG | | |
| | | | GCCUAGCGUGGCCU | | |
| | | | ACCUGCAGAGCAAG | | |
| | | | GGCAAAGACAUCAG | | |
| | | | CAACGGAUACGGCG | | |
| | | | CCAGCUACGGCGAU | | |
| | | | CAGGACAUCGUGAA | | |
| | | | AUACGUGGACGUGG | | |
| | | | GCGCCACCUAUUAC | | |
| | | | UUCAACAAGAACAU | | |
| | | | GAGCACCUACGUGG | | |
| | | | ACUACAAGAUCAAC | | |
| | | | CUGCUGGACAAGAA | | |
| | | | CGACUUCACCCGCG | | |
| | | | ACGCCGGCAUCAAC | | |
| | | | ACCGAUGAUAUUGU | | |
| | | | GGCCCUGGGCCUCG | | |
| | | | UGUACCAGUUU | | |
| SEQ ID NO: | | 7 | 8 | 3 | 4 |

SEQ ID NO: 163 consists of from 5′ end to 3′ end, 5′ UTR SEQ
ID NO: 3, mRNA ORF SEQ ID NO: 8, and 3′ UTR SEQ ID NO: 4.

| | St_OmpF_ | METPAQLLFLLLLWL | AUGGAAACCCCUGC | GGGAAA | UGAUAA |
|---|---|---|---|---|---|
| | nIgK_cHis | PDTTGAEIYNKDGNK | UCAGCUGCUGUUCC | UAAGAG | UAGGCU |
| | variant | LDLYGKAVGRHVWT | UGCUGCUGCUGUGG | AGAAAA | GGAGCC |
| | | TTGDSKNADQTYAQI | CUGCCUGAUACAAC | GAAGAG | UCGGUG |
| | | GFKGETQINTDLTGF | AGGCGCCGAGAUCU | UAAGAA | GCCAUG |
| | | GQWEYRTKADRAEG | ACAACAAGGACGGC | GAAAUA | CUUCUU |
| | | EQQNSNLVRLAFAGL | AACAAGCUGGACCU | UAAGAG | GCCCCU |
| | | KYAEVGSIDYGRNY | GUACGGCAAAGCCG | CCACC | UGGGCC |
| | | GIVYDVESYTDMAPY | UGGGCAGACACGUG | | UCCCCC |
| | | FSGETWGGAYTDNY | UGGACAACCACCGG | | CAGCCC |
| | | MTSRAGGLLTYRNS | CGAUAGCAAGAACG | | CUCCUC |
| | | DFFGLVDGLSFGIQY | CCGACCAGACAUAU | | CCCUUC |
| | | QGKNQDNHSINSQN | GCCCAGAUCGGCUU | | CUGCAC |
| | | GDGVGYTMAYEFDG | CAAGGGCGAGACAC | | CCGUAC |
| | | FGVTAAYSNSKRTND | AGAUCAACACCGAC | | CCCCGU |
| | | QQDRDGNGDRAESW | CUGACCGGCUUUGG | | GGUCUU |
| | | AVGAKYDANNVYLA | CCAGUGGGAGUACA | | UGAAUA |
| | | AVYAETRNMSIVENT | GAACAAAGGCCGAC | | AAGUCU |
| | | VTDTVEMANKTQNL | AGAGCCGAGGGCGA | | GAGUGG |
| | | EVVAQYQFDFGLRPA | GCAGCAGAAUUCUA | | GCGGC |
| | | ISYVQSKGKQLNGAD | AUCUUGUGCGGCUG | | |
| | | GSADLAKYIQAGATY | GCCUUCGCCGGCCU | | |
| | | YFNKNMNVWVDYR | GAAGUAUGCUGAAG | | |
| | | FNLLDENDYSSSYVG | UGGGCAGCAUCGAC | | |
| | | TDDQAAVGITYQFHH | UACGGCCGGAAUUA | | |
| | | HHHH | CGGCAUCGUGUACG | | |
| | | | ACGUGGAAAGCUAC | | |
| | | | ACCGACAUGGCCCC | | |
| | | | UUACUUCAGCGGCG | | |
| | | | AAACAUGGGGCGGA | | |
| | | | GCCUACACCGAUAA | | |
| | | | CUACAUGACCAGCA | | |
| | | | GAGCCGGCGGACUG | | |
| | | | CUGACCUACAGAAA | | |
| | | | CAGCGAUUUCUUCG | | |
| | | | GCCUGGUGGACGGC | | |
| | | | CUGAGCUUCGGCAU | | |
| | | | UCAGUACCAGGGCA | | |
| | | | AGAACCAGGACAAU | | |
| | | | CACAGCAUCAACAG | | |
| | | | CCAGAACGGCGACG | | |
| | | | GCGUGGGCUACACA | | |
| | | | AUGGCCUACGAGUU | | |
| | | | CGAUGGCUUUGGCG | | |
| | | | UGACAGCCGCCUAC | | |
| | | | AGCAACUCCAAGAG | | |
| | | | GACCAACGACCAGC | | |
| | | | AGGACAGAGAUGGC | | |
| | | | AACGGCGAUAGAGC | | |
| | | | CGAAUCUUGGGCCG | | |
| | | | UGGGCGCCAAAUAC | | |
| | | | GACGCCAACAAUGU | | |
| | | | GUAUCUGGCCGCCG | | |
| | | | UGUACGCCGAGACA | | |
| | | | CGGAACAUGAGCAU | | |
| | | | CGUGGAGAACACCG | | |
| | | | UGACCGACACCGUG | | |
| | | | GAAAUGGCCAACAA | | |
| | | | GACCCAGAACCUGG | | |
| | | | AAGUGGUGGCCCAG | | |
| | | | UACCAGUUCGACUU | | |
| | | | UGGACUGAGGCCCG | | |
| | | | CCAUCAGCUACGUG | | |
| | | | CAGUCUAAGGGCAA | | |
| | | | GCAGCUGAAUGGCG | | |
| | | | CCGAUGGCUCUGCA | | |
| | | | GACCUGGCCAAGUA | | |
| | | | UAUUCAGGCUGGCG | | |
| | | | CCACCUACUACUUC | | |
| | | | AACAAGAACAUGAA | | |
| | | | CGUGUGGGUCGACU | | |
| | | | ACCGGUUCAACCUG | | |
| | | | CUGGACGAGAACGA | | |
| | | | CUACAGCAGCUCCU | | |
| | | | ACGUGGGCACCGAU | | |
| | | | GAUCAGGCCGCUGU | | |
| | | | GGGCAUCACCUACC | | |
| | | | AGUUCCACCACCAC | | |
| | | | CAUCACCAC | | |
| SEQ ID NO: | | 9 | 10 | 3 | 4 |

SEQ ID NO: 164 consists of from 5′ end to 3′ end, 5′ UTR SEQ
ID NO: 3, mRNA ORF SEQ ID NO: 10, and 3′ UTR SEQ ID NO: 4.

| | St_OmpF_ | METPAQLLFLLLLWL | AUGGAAACCCCUGC | GGGAAA | UGAUAA |
|---|---|---|---|---|---|
| | nIgK | PDTTGAEIYNKDGNK | UCAGCUGCUGUUCC | UAAGAG | UAGGCU |
| | variant | LDLYGKAVGRHVWT | UGCUGCUGCUGUGG | AGAAAA | GGAGCC |
| | | TTGDSKNADQTYAQI | CUGCCUGAUACAAC | GAAGAG | UCGGUG |
| | | GFKGETQINTDLTGF | AGGCGCCGAGAUCU | UAAGAA | GCCAUG |
| | | GQWEYRTKADRAEG | ACAACAAGGACGGC | GAAAUA | CUUCUU |
| | | EQQNSNLVRLAFAGL | AACAAGCUGGACCU | UAAGAG | GCCCCU |
| | | KYAEVGSIDYGRNY | GUACGGCAAAGCCG | CCACC | UGGGCC |
| | | GIVYDVESYTDMAPY | UGGGCAGACACGUG | | UCCCCC |
| | | FSGETWGGAYTDNY | UGGACAACCACCGG | | CAGCCC |
| | | MTSRAGGLLTYRNS | CGAUAGCAAGAACG | | CUCCUC |
| | | DFFGLVDGLSFGIQY | CCGACCAGACAUAU | | CCCUUC |
| | | QGKNQDNHSINSQN | GCCCAGAUCGGCUU | | CUGCAC |
| | | GDGVGYTMAYEFDG | CAAGGGCGAGACAC | | CCGUAC |
| | | FGVTAAYSNSKRTND | AGAUCAACACCGAC | | CCCCGU |
| | | QQDRDGNGDRAESW | CUGACCGGCUUUGG | | GGUCUU |
| | | AVGAKYDANNVYLA | CCAGUGGGAGUACA | | UGAAUA |
| | | AVYAETRNMSIVENT | GAACAAAGGCCGAC | | AAGUCU |
| | | VTDTVEMANKTQNL | AGAGCCGAGGGCGA | | GAGUGG |
| | | EVVAQYQFDFGLRPA | GCAGCAGAAUUCUA | | GCGGC |
| | | ISYVQSKGKQLNGAD | AUCUUGUGCGGCUG | | |
| | | GSADLAKYIQAGATY | GCCUUCGCCGGCCU | | |
| | | YFNKNMNVWVDYR | GAAGUAUGCUGAAG | | |
| | | FNLLDENDYSSSYVG | UGGGCAGCAUCGAC | | |
| | | TDDQAAVGITYQF | UACGGCCGGAAUUA | | |
| | | | CGGCAUCGUGUACG | | |
| | | | ACGUGGAAAGCUAC | | |
| | | | ACCGACAUGGCCCC | | |
| | | | UUACUUCAGCGGCG | | |
| | | | AAACAUGGGGCGGA | | |
| | | | GCCUACACCGAUAA | | |
| | | | CUACAUGACCAGCA | | |
| | | | GAGCCGGCGGACUG | | |
| | | | CUGACCUACAGAAA | | |
| | | | CAGCGAUUUCUUCG | | |
| | | | GCCUGGUGGACGGC | | |
| | | | CUGAGCUUCGGCAU | | |
| | | | UCAGUACCAGGGCA | | |
| | | | AGAACCAGGACAAU | | |
| | | | CACAGCAUCAACAG | | |
| | | | CCAGAACGGCGACG | | |
| | | | GCGUGGGCUACACA | | |
| | | | AUGGCCUACGAGUU | | |
| | | | CGAUGGCUUUGGCG | | |
| | | | UGACAGCCGCCUAC | | |
| | | | AGCAACUCCAAGAG | | |
| | | | GACCAACGACCAGC | | |
| | | | AGGACAGAGAUGGC | | |
| | | | AACGGCGAUAGAGC | | |
| | | | CGAAUCUUGGGCCG | | |
| | | | UGGGCGCCAAAUAC | | |
| | | | GACGCCAACAAUGU | | |
| | | | GUAUCUGGCCGCCG | | |
| | | | UGUACGCCGAGACA | | |
| | | | CGGAACAUGAGCAU | | |
| | | | CGUGGAGAACACCG | | |
| | | | UGACCGACACCGUG | | |
| | | | GAAAUGGCCAACAA | | |
| | | | GACCCAGAACCUGG | | |
| | | | AAGUGGUGGCCCAG | | |
| | | | UACCAGUUCGACUU | | |
| | | | UGGACUGAGGCCCG | | |
| | | | CCAUCAGCUACGUG | | |
| | | | CAGUCUAAGGGCAA | | |
| | | | GCAGCUGAAUGGCG | | |
| | | | CCGAUGGCUCUGCA | | |
| | | | GACCUGGCCAAGUA | | |
| | | | UAUUCAGGCUGGCG | | |
| | | | CCACCUACUACUUC | | |
| | | | AACAAGAACAUGAA | | |
| | | | CGUGUGGGUCGACU | | |
| | | | ACCGGUUCAACCUG | | |
| | | | CUGGACGAGAACGA | | |
| | | | CUACAGCAGCUCCU | | |
| | | | ACGUGGGCACCGAU | | |
| | | | GAUCAGGCCGCUGU | | |
| | | | GGGCAUCACCUACC | | |
| | | | AGUUC | | |
| SEQ ID NO: | | 11 | 12 | 3 | 4 |

SEQ ID NO: 165 consists of from 5′ end to 3′ end, 5′ UTR SEQ
ID NO: 3, mRNA ORF SEQ ID NO: 12, and 3′ UTR SEQ ID NO: 4.

| | St_OmpF_ | METPAQLLFLLLLWL | AUGGAAACCCCUGC | GGGAAA | UGAUAA |
|---|---|---|---|---|---|
| | NGM_nIg | PDTTGAEIYNKDGNK | UCAGCUGCUGUUCC | UAAGAG | UAGGCU |
| | K_cHis | LDLYGKAVGRHVWT | UGCUGCUGCUGUGG | AGAAAA | GGAGCC |
| | variant | TTGDSKNADQTYAQI | CUGCCUGAUACAAC | GAAGAG | UCGGUG |
| | | GFKGETQINTDLTGF | AGGCGCCGAGAUCU | UAAGAA | GCCAUG |
| | | GQWEYRTKADRAEG | ACAACAAGGACGGC | GAAAUA | CUUCUU |
| | | EQQNSNLVRLAFAGL | AACAAGCUGGACCU | UAAGAG | GCCCCU |
| | | KYAEVGSIDYGRNY | GUACGGCAAAGCCG | CCACC | UGGGCC |
| | | GIVYDVESYTDMAPY | UGGGCAGACACGUG | | UCCCCC |
| | | FSGETWGGAYTDNY | UGGACAACCACCGG | | CAGCCC |
| | | MTSRAGGLLTYRNS | CGAUAGCAAGAACG | | CUCCUC |
| | | DFFGLVDGLSFGIQY | CCGACCAGACAUAU | | CCCUUC |
| | | QGKNQDNHSINSQN | GCCCAGAUCGGCUU | | CUGCAC |
| | | GDGVGYTMAYEFDG | CAAGGGCGAGACAC | | CCGUAC |
| | | FGVTAAYSNSKRTND | AGAUCAACACCGAC | | CCCCGU |
| | | QQDRDGNGDRAESW | CUGACCGGCUUUGG | | GGUCUU |
| | | AVGAKYDANNVYLA | CCAGUGGGAGUACA | | UGAAUA |
| | | AVYAETRNMAIVEN | GAACAAAGGCCGAC | | AAGUCU |
| | | TVTDTVEMANKAQN | AGAGCCGAGGGCGA | | GAGUGG |
| | | LEVVAQYQFDFGLRP | GCAGCAGAAUUCUA | | GCGGC |
| | | AISYVQSKGKQLNGA | AUCUUGUGCGGCUG | | |
| | | DGSADLAKYIQAGAT | GCCUUCGCCGGCCU | | |
| | | YYFNKNMNVWVDY | GAAGUAUGCUGAAG | | |
| | | RFNLLDENDYSSSYV | UGGGCAGCAUCGAC | | |
| | | GTDDQAAVGITYQFH | UACGGCCGGAAUUA | | |
| | | HHHHH | CGGCAUCGUGUACG | | |
| | | | ACGUGGAAAGCUAC | | |
| | | | ACCGACAUGGCCCC | | |
| | | | UUACUUCAGCGGCG | | |
| | | | AAACAUGGGGCGGA | | |
| | | | GCCUACACCGAUAA | | |
| | | | CUACAUGACCAGCA | | |
| | | | GAGCCGGCGGACUG | | |
| | | | CUGACCUACAGAAA | | |
| | | | CAGCGAUUUCUUCG | | |
| | | | GCCUGGUGGACGGC | | |
| | | | CUGAGCUUCGGCAU | | |
| | | | UCAGUACCAGGGCA | | |
| | | | AGAACCAGGACAAU | | |
| | | | CACAGCAUCAACAG | | |
| | | | CCAGAACGGCGACG | | |
| | | | GCGUGGGCUACACA | | |
| | | | AUGGCCUACGAGUU | | |
| | | | CGAUGGCUUUGGCG | | |
| | | | UGACAGCCGCCUAC | | |
| | | | AGCAACUCCAAGAG | | |
| | | | GACCAACGACCAGC | | |
| | | | AGGACAGAGAUGGC | | |
| | | | AACGGCGAUAGAGC | | |
| | | | CGAAUCUUGGGCCG | | |
| | | | UGGGCGCCAAAUAC | | |
| | | | GACGCCAACAAUGU | | |
| | | | GUAUCUGGCCGCCG | | |
| | | | UGUACGCCGAGACA | | |
| | | | AGAAACAUGGCCAU | | |
| | | | CGUGGAGAACACCG | | |
| | | | UGACCGACACCGUG | | |
| | | | GAAAUGGCCAACAA | | |
| | | | GGCCCAGAACCUGG | | |
| | | | AAGUGGUGGCCCAG | | |
| | | | UACCAGUUCGACUU | | |
| | | | UGGACUGAGGCCCG | | |
| | | | CCAUCAGCUACGUG | | |
| | | | CAGUCUAAGGGCAA | | |
| | | | GCAGCUGAAUGGCG | | |
| | | | CCGAUGGCUCUGCA | | |
| | | | GACCUGGCCAAGUA | | |
| | | | UAUUCAGGCUGGCG | | |
| | | | CCACCUACUACUUC | | |
| | | | AACAAGAACAUGAA | | |
| | | | CGUGUGGGUCGACU | | |
| | | | ACCGGUUCAACCUG | | |
| | | | CUGGACGAGAACGA | | |
| | | | CUACAGCAGCUCCU | | |
| | | | ACGUGGGCACCGAU | | |
| | | | GAUCAGGCCGCUGU | | |
| | | | GGGCAUCACCUACC | | |
| | | | AGUUCCACCACCAC | | |
| | | | CAUCACCAC | | |
| SEQ ID NO: | | 13 | 14 | 3 | 4 |

SEQ ID NO: 166 consists of from 5′ end to 3′ end, 5′ UTR SEQ
ID NO: 3, mRNA ORF SEQ ID NO: 14, and 3′ UTR SEQ ID NO: 4.

| | St_OmpF_ | METPAQLLFLLLLWL | AUGGAAACCCCUGC | GGGAAA | UGAUAA |
|---|---|---|---|---|---|
| | NGM_nIg | PDTTGAEIYNKDGNK | UCAGCUGCUGUUCC | UAAGAG | UAGGCU |
| | K variant | LDLYGKAVGRHVWT | UGCUGCUGCUGUGG | AGAAAA | GGAGCC |
| | | TTGDSKNADQTYAQI | CUGCCUGAUACAAC | GAAGAG | UCGGUG |
| | | GFKGETQINTDLTGF | AGGCGCCGAGAUCU | UAAGAA | GCCAUG |
| | | GQWEYRTKADRAEG | ACAACAAGGACGGC | GAAAUA | CUUCUU |
| | | EQQNSNLVRLAFAGL | AACAAGCUGGACCU | UAAGAG | GCCCCU |
| | | KYAEVGSIDYGRNY | GUACGGCAAAGCCG | CCACC | UGGGCC |
| | | GIVYDVESYTDMAPY | UGGGCAGACACGUG | | UCCCCC |
| | | FSGETWGGAYTDNY | UGGACAACCACCGG | | CAGCCC |
| | | MTSRAGGLLTYRNS | CGAUAGCAAGAACG | | CUCCUC |
| | | DFFGLVDGLSFGIQY | CCGACCAGACAUAU | | CCCUUC |
| | | QGKNQDNHSINSQN | GCCCAGAUCGGCUU | | CUGCAC |
| | | GDGVGYTMAYEFDG | CAAGGGCGAGACAC | | CCGUAC |
| | | FGVTAAYSNSKRTND | AGAUCAACACCGAC | | CCCCGU |
| | | QQDRDGNGDRAESW | CUGACCGGCUUUGG | | GGUCUU |
| | | AVGAKYDANNVYLA | CCAGUGGGAGUACA | | UGAAUA |
| | | AVYAETRNMAIVEN | GAACAAAGGCCGAC | | AAGUCU |
| | | TVTDTVEMANKAQN | AGAGCCGAGGGCGA | | GAGUGG |
| | | LEVVAQYQFDFGLRP | GCAGCAGAAUUCUA | | GCGGC |
| | | AISYVQSKGKQLNGA | AUCUUGUGCGGCUG | | |
| | | DGSADLAKYIQAGAT | GCCUUCGCCGGCCU | | |
| | | YYFNKNMNVWVDY | GAAGUAUGCUGAAG | | |
| | | RFNLLDENDYSSSYV | UGGGCAGCAUCGAC | | |
| | | GTDDQAAVGITYQF | UACGGCCGGAAUUA | | |
| | | | CGGCAUCGUGUACG | | |
| | | | ACGUGGAAAGCUAC | | |
| | | | ACCGACAUGGCCCC | | |
| | | | UUACUUCAGCGGCG | | |
| | | | AAACAUGGGGCGGA | | |
| | | | GCCUACACCGAUAA | | |
| | | | CUACAUGACCAGCA | | |
| | | | GAGCCGGCGGACUG | | |
| | | | CUGACCUACAGAAA | | |
| | | | CAGCGAUUUCUUCG | | |
| | | | GCCUGGUGGACGGC | | |
| | | | CUGAGCUUCGGCAU | | |
| | | | UCAGUACCAGGGCA | | |
| | | | AGAACCAGGACAAU | | |
| | | | CACAGCAUCAACAG | | |
| | | | CCAGAACGGCGACG | | |
| | | | GCGUGGGCUACACA | | |
| | | | AUGGCCUACGAGUU | | |
| | | | CGAUGGCUUUGGCG | | |
| | | | UGACAGCCGCCUAC | | |
| | | | AGCAACUCCAAGAG | | |
| | | | GACCAACGACCAGC | | |
| | | | AGGACAGAGAUGGC | | |
| | | | AACGGCGAUAGAGC | | |
| | | | CGAAUCUUGGGCCG | | |
| | | | UGGGCGCCAAAUAC | | |
| | | | GACGCCAACAAUGU | | |
| | | | GUAUCUGGCCGCCG | | |
| | | | UGUACGCCGAGACA | | |
| | | | AGAAACAUGGCCAU | | |
| | | | CGUGGAGAACACCG | | |
| | | | UGACCGACACCGUG | | |
| | | | GAAAUGGCCAACAA | | |
| | | | GGCCCAGAACCUGG | | |
| | | | AAGUGGUGGCCCAG | | |
| | | | UACCAGUUCGACUU | | |
| | | | UGGACUGAGGCCCG | | |
| | | | CCAUCAGCUACGUG | | |
| | | | CAGUCUAAGGGCAA | | |
| | | | GCAGCUGAAUGGCG | | |
| | | | CCGAUGGCUCUGCA | | |
| | | | GACCUGGCCAAGUA | | |
| | | | UAUUCAGGCUGGCG | | |
| | | | CCACCUACUACUUC | | |
| | | | AACAAGAACAUGAA | | |
| | | | CGUGUGGGUCGACU | | |
| | | | ACCGGUUCAACCUG | | |
| | | | CUGGACGAGAACGA | | |
| | | | CUACAGCAGCUCCU | | |
| | | | ACGUGGGCACCGAU | | |
| | | | GAUCAGGCCGCUGU | | |
| | | | GGGCAUCACCUACC | | |
| | | | AGUUC | | |
| SEQ ID NO: | | 15 | 16 | 3 | 4 |

SEQ ID NO: 167 consists of from 5′ end to 3′ end, 5′ UTR SEQ
ID NO: 3, mRNA ORF SEQ ID NO: 16, and 3′ UTR SEQ ID NO: 4.

| | Stm_Omp | METPAQLLFLLLLWL | AUGGAAACCCCUGC | GGGAAA | UGAUAA |
|---|---|---|---|---|---|
| | D_nIgK_c | PDTTGAEVYNKDGN | UCAGCUGCUGUUCC | UAAGAG | UAGGCU |
| | His variant | KLDLYGKVHAQHYF | UGCUGCUGCUGUGG | AGAAAA | GGAGCC |
| | | SDDNGSDGDKTYAR | CUGCCUGAUACAAC | GAAGAG | UCGGUG |
| | | LGFKGETQINDQLTG | AGGCGCCGAGGUGU | UAAGAA | GCCAUG |
| | | FGQWEYEFKGNRTES | ACAACAAGGACGGC | GAAAUA | CUUCUU |
| | | QGADKDKTRLAFAG | AACAAGCUGGACCU | UAAGAG | GCCCCU |
| | | LKFADYGSFDYGRN | GUACGGCAAAGUGC | CCACC | UGGGCC |
| | | YGVAYDIGAWTDVL | ACGCCCAGCACUAC | | UCCCCC |
| | | PEFGGDTWTQTDVF | UUCAGCGACGACAA | | CAGCCC |
| | | MTGRTTGVATYRNT | UGGCAGCGACGGCG | | CUCCUC |
| | | DFFGLVEGLNFAAQY | ACAAGACAUAUGCC | | CCCUUC |
| | | QGKNDRDGAYESNG | CGGCUUGGCUUCAA | | CUGCAC |
| | | DGFGLSATYEYEGFG | GGGCGAGACACAGA | | CCGUAC |
| | | VGAAYAKSDRTNNQ | UCAACGACCAGCUG | | CCCCGU |
| | | VKAASNLNAAGKNA | ACCGGCUUUGGCCA | | GGUCUU |
| | | EVWAAGLKYDANNI | GUGGGAGUACGAGU | | UGAAUA |
| | | YLATTYSETLNMTTF | UCAAGGGCAACAGA | | AAGUCU |
| | | GEDAAGDAFIANKTQ | ACCGAGAGCCAGGG | | GAGUGG |
| | | NFEAVAQYQFDFGLR | CGCCGACAAGGACA | | GCGGC |
| | | PSIAYLKSKGKNLGT | AGACCAGACUGGCC | | |
| | | YGDQDLVEYIDVGA | UUUGCCGGCCUGAA | | |
| | | TYYFNKNMSTFVDY | GUUCGCCGAUUACG | | |
| | | KINLLDDSDFTKAAK | GCAGCUUUGACUAC | | |
| | | VSTDNIVAVGLNYQF | GGCCGGAAUUACGG | | |
| | | HHHHHH | CGUGGCCUACGAUA | | |
| | | | UCGGAGCCUGGACA | | |
| | | | GAUGUGCUGCCUGA | | |
| | | | GUUUGGCGGCGACA | | |
| | | | CCUGGACACAGACC | | |
| | | | GACGUGUUCAUGAC | | |
| | | | CGGCAGAACAACUG | | |
| | | | GCGUGGCCACCUAC | | |
| | | | CGGAACACCGAUUU | | |
| | | | CUUUGGCCUGGUGG | | |
| | | | AAGGCCUGAACUUU | | |
| | | | GCCGCUCAGUACCA | | |
| | | | GGGCAAGAACGACA | | |
| | | | GAGAUGGCGCCUAC | | |
| | | | GAGUCUAACGGCGA | | |
| | | | CGGCUUUGGACUGA | | |
| | | | GCGCCACCUACGAG | | |
| | | | UACGAAGGCUUUGG | | |
| | | | AGUGGGCGCUGCCU | | |
| | | | ACGCCAAGAGCGAC | | |
| | | | AGGACCAACAAUCA | | |
| | | | AGUGAAGGCCGCCA | | |
| | | | GCAACCUGAACGCC | | |
| | | | GCUGGAAAGAAUGC | | |
| | | | CGAAGUGUGGGCCG | | |
| | | | CUGGACUGAAGUAC | | |
| | | | GACGCCAACAACAU | | |
| | | | CUACCUGGCCACCA | | |
| | | | CCUACAGCGAGACA | | |
| | | | CUGAACAUGACCAC | | |
| | | | CUUCGGCGAAGAUG | | |
| | | | CCGCUGGCGACGCC | | |
| | | | UUUAUCGCCAACAA | | |
| | | | GACCCAGAACUUCG | | |
| | | | AGGCUGUGGCCCAG | | |
| | | | UACCAGUUCGACUU | | |
| | | | CGGACUGAGGCCCU | | |
| | | | CUAUCGCCUACCUG | | |
| | | | AAGUCCAAGGGAAA | | |
| | | | GAACCUGGGCACCU | | |
| | | | ACGGCGACCAGGAC | | |
| | | | CUGGUUGAGUAUAU | | |
| | | | CGAUGUGGGAGCCA | | |
| | | | CCUACUACUUCAAC | | |
| | | | AAGAAUAUGAGCAC | | |
| | | | CUUCGUGGACUACA | | |
| | | | AGAUCAACCUGCUG | | |
| | | | GACGACAGCGACUU | | |
| | | | CACCAAAGCCGCCA | | |
| | | | AGGUGUCCACCGAC | | |
| | | | AACAUUGUGGCCGU | | |
| | | | GGGCCUGAAUUACC | | |
| | | | AGUUCCACCACCAC | | |
| | | | CAUCACCAC | | |
| SEQ ID NO: | | 17 | 18 | 3 | 4 |

SEQ ID NO: 168 consists of from 5′ end to 3′ end, 5′ UTR SEQ
ID NO: 3, mRNA ORF SEQ ID NO: 18, and 3′ UTR SEQ ID NO: 4.

| | Stm_Omp | METPAQLLFLLLLWL | AUGGAAACCCCUGC | GGGAAA | UGAUAA |
|---|---|---|---|---|---|
| | D_nIgK | PDTTGAEVYNKDGN | UCAGCUGCUGUUCC | UAAGAG | UAGGCU |
| | variant | KLDLYGKVHAQHYF | UGCUGCUGCUGUGG | AGAAAA | GGAGCC |
| | | SDDNGSDGDKTYAR | CUGCCUGAUACAAC | GAAGAG | UCGGUG |
| | | LGFKGETQINDQLTG | AGGCGCCGAGGUGU | UAAGAA | GCCAUG |
| | | FGQWEYEFKGNRTES | ACAACAAGGACGGC | GAAAUA | CUUCUU |
| | | QGADKDKTRLAFAG | AACAAGCUGGACCU | UAAGAG | GCCCCU |
| | | LKFADYGSFDYGRN | GUACGGCAAAGUGC | CCACC | UGGGCC |
| | | YGVAYDIGAWTDVL | ACGCCCAGCACUAC | | UCCCCC |
| | | PEFGGDTWTQTDVF | UUCAGCGACGACAA | | CAGCCC |
| | | MTGRTTGVATYRNT | UGGCAGCGACGGCG | | CUCCUC |
| | | DFFGLVEGLNFAAQY | ACAAGACAUAUGCC | | CCCUUC |
| | | QGKNDRDGAYESNG | CGGCUUGGCUUCAA | | CUGCAC |
| | | DGFGLSATYEYEGFG | GGGCGAGACACAGA | | CCGUAC |
| | | VGAAYAKSDRTNNQ | UCAACGACCAGCUG | | CCCCGU |
| | | VKAASNLNAAGKNA | ACCGGCUUUGGCCA | | GGUCUU |
| | | EVWAAGLKYDANNI | GUGGGAGUACGAGU | | UGAAUA |
| | | YLATTYSETLNMTTF | UCAAGGGCAACAGA | | AAGUCU |
| | | GEDAAGDAFIANKTQ | ACCGAGAGCCAGGG | | GAGUGG |
| | | NFEAVAQYQFDFGLR | CGCCGACAAGGACA | | GCGGC |
| | | PSIAYLKSKGKNLGT | AGACCAGACUGGCC | | |
| | | YGDQDLVEYIDVGA | UUUGCCGGCCUGAA | | |
| | | TYYFNKNMSTFVDY | GUUCGCCGAUUACG | | |
| | | KINLLDDSDFTKAAK | GCAGCUUUGACUAC | | |
| | | VSTDNIVAVGLNYQF | GGCCGGAAUUACGG | | |
| | | | CGUGGCCUACGAUA | | |
| | | | UCGGAGCCUGGACA | | |
| | | | GAUGUGCUGCCUGA | | |
| | | | GUUUGGCGGCGACA | | |
| | | | CCUGGACACAGACC | | |
| | | | GACGUGUUCAUGAC | | |
| | | | CGGCAGAACAACUG | | |
| | | | GCGUGGCCACCUAC | | |
| | | | CGGAACACCGAUUU | | |
| | | | CUUUGGCCUGGUGG | | |
| | | | AAGGCCUGAACUUU | | |
| | | | GCCGCUCAGUACCA | | |
| | | | GGGCAAGAACGACA | | |
| | | | GAGAUGGCGCCUAC | | |
| | | | GAGUCUAACGGCGA | | |
| | | | CGGCUUUGGACUGA | | |
| | | | GCGCCACCUACGAG | | |
| | | | UACGAAGGCUUUGG | | |
| | | | AGUGGGCGCUGCCU | | |
| | | | ACGCCAAGAGCGAC | | |
| | | | AGGACCAACAAUCA | | |
| | | | AGUGAAGGCCGCCA | | |
| | | | GCAACCUGAACGCC | | |
| | | | GCUGGAAAGAAUGC | | |
| | | | CGAAGUGUGGGCCG | | |
| | | | CUGGACUGAAGUAC | | |
| | | | GACGCCAACAACAU | | |
| | | | CUACCUGGCCACCA | | |
| | | | CCUACAGCGAGACA | | |
| | | | CUGAACAUGACCAC | | |
| | | | CUUCGGCGAAGAUG | | |
| | | | CCGCUGGCGACGCC | | |
| | | | UUUAUCGCCAACAA | | |
| | | | GACCCAGAACUUCG | | |
| | | | AGGCUGUGGCCCAG | | |
| | | | UACCAGUUCGACUU | | |
| | | | CGGACUGAGGCCCU | | |
| | | | CUAUCGCCUACCUG | | |
| | | | AAGUCCAAGGGAAA | | |
| | | | GAACCUGGGCACCU | | |
| | | | ACGGCGACCAGGAC | | |
| | | | CUGGUUGAGUAUAU | | |
| | | | CGAUGUGGGAGCCA | | |
| | | | CCUACUACUUCAAC | | |
| | | | AAGAAUAUGAGCAC | | |
| | | | CUUCGUGGACUACA | | |
| | | | AGAUCAACCUGCUG | | |
| | | | GACGACAGCGACUU | | |
| | | | CACCAAAGCCGCCA | | |
| | | | AGGUGUCCACCGAC | | |
| | | | AACAUUGUGGCCGU | | |
| | | | GGGCCUGAAUUACC | | |
| | | | AGUUC | | |
| SEQ ID NO: | | 19 | 20 | 3 | 4 |

SEQ ID NO: 169 consists of from 5′ end to 3′ end, 5′ UTR SEQ
ID NO: 3, mRNA ORF SEQ ID NO: 20, and 3′ UTR SEQ ID NO: 4.

| | St_OmpC_ | METPAQLLFLLLLWL | AUGGAAACCCCUGC | GGGAAA | UGAUAA |
|---|---|---|---|---|---|
| | NGM_nIg | PDTTGAEIYNKDGNK | UCAGCUGCUGUUCC | UAAGAG | UAGGCU |
| | k_cHis | LDLFGKVDGLHYFSD | UGCUGCUGCUGUGG | AGAAAA | GGAGCC |
| | | DKGSDGDQTYMRIG | CUGCCUGAUACAAC | GAAGAG | UCGGUG |
| | | FKGETQVNDQLTGY | AGGCGCCGAGAUCU | UAAGAA | GCCAUG |
| | | GQWEYQIQGNQAEG | ACAACAAGGACGGC | GAAAUA | CUUCUU |
| | | SNDAWTRVAFAGLK | AACAAGCUGGACCU | UAAGAG | GCCCCU |
| | | FADAGSFDYGRNYG | GUUCGGCAAGGUGG | CCACC | UGGGCC |
| | | VTYDVTSWTDVLPEF | ACGGCCUGCACUAC | | UCCCCC |
| | | GGDTYGADNFMQQR | UUCAGCGACGAUAA | | CAGCCC |
| | | GNGYATYRNTDFFG | GGGCUCCGACGGCG | | CUCCUC |
| | | LVDGLDFALQYQGK | ACCAGACCUACAUG | | CCCUUC |
| | | NGAVSGENTNGRSLL | AGAAUCGGCUUCAA | | CUGCAC |
| | | NQNGDGYGGSLTYAI | GGGCGAGACACAAG | | CCGUAC |
| | | GEGFSVGGAITTSKR | UGAACGACCAGCUG | | CCCCGU |
| | | TADQNNAANARLYG | ACAGGCUACGGCCA | | GGUCUU |
| | | NGDRATVYTGGLKY | GUGGGAGUAUCAGA | | UGAAUA |
| | | DANNIYLAAQYSQTY | UCCAGGGCAAUCAG | | AAGUCU |
| | | NAARFGTSNGANPST | GCCGAGGGCAGCAA | | GAGUGG |
| | | SYGFANKAQNFEVV | CGACGCAUGGACCA | | GCGGC |
| | | AQYQFDFGLRPSVAY | GAGUGGCUUUUGCC | | |
| | | LQSKGKDISNGYGAS | GGCCUGAAGUUUGC | | |
| | | YGDQDIVKYVDVGA | CGAUGCCGGCAGCU | | |
| | | TYYFNKNMSTYVDY | UUGACUACGGCAGA | | |
| | | KINLLDKNDFTRDAG | AAUUACGGCGUGAC | | |
| | | INTDDIVALGLVYQF | CUACGACGUGACCU | | |
| | | HHHHHH | CCUGGACAGAUGUG | | |
| | | | CUGCCUGAGUUUGG | | |
| | | | CGGCGAUACCUACG | | |
| | | | GCGCCGACAACUUC | | |
| | | | AUGCAGCAGAGAGG | | |
| | | | CAACGGCUACGCCA | | |
| | | | CCUACCGGAACACC | | |
| | | | GAUUUCUUCGGCCU | | |
| | | | GGUGGAUGGCCUGG | | |
| | | | AUUUCGCCCUGCAG | | |
| | | | UACCAGGGCAAGAA | | |
| | | | UGGCGCUGUGUCCG | | |
| | | | GCGAGAACACCAAC | | |
| | | | GGCAGAAGCCUGCU | | |
| | | | GAACCAGAACGGCG | | |
| | | | ACGGAUAUGGCGGC | | |
| | | | AGCCUGACAUAUGC | | |
| | | | CAUCGGCGAGGGCU | | |
| | | | UUUCUGUCGGCGGA | | |
| | | | GCCAUCACCACCAG | | |
| | | | CAAGAGAACAGCCG | | |
| | | | ACCAGAACAACGCC | | |
| | | | GCCAACGCCAGACU | | |
| | | | GUACGGCAACGGCG | | |
| | | | AUAGAGCCACAGUG | | |
| | | | UAUACCGGCGGACU | | |
| | | | GAAGUACGACGCCA | | |
| | | | ACAACAUCUACCUG | | |
| | | | GCCGCUCAGUACAG | | |
| | | | CCAGACAUACAACG | | |
| | | | CCGCCAGAUUCGGC | | |
| | | | ACCAGCAACGGCGC | | |
| | | | AAAUCCCAGCACAA | | |
| | | | GCUACGGCUUCGCC | | |
| | | | AACAAGGCCCAGAA | | |
| | | | CUUUGAGGUGGUGG | | |
| | | | CCCAGUACCAGUUC | | |
| | | | GACUUCGGACUGAG | | |
| | | | GCCUAGCGUGGCCU | | |
| | | | ACCUGCAGAGCAAG | | |
| | | | GGCAAAGACAUCAG | | |
| | | | CAACGGAUACGGCG | | |
| | | | CCAGCUACGGCGAU | | |
| | | | CAGGACAUCGUGAA | | |
| | | | AUACGUGGACGUGG | | |
| | | | GCGCCACCUAUUAC | | |
| | | | UUCAACAAGAACAU | | |
| | | | GAGCACCUACGUGG | | |
| | | | ACUACAAGAUCAAC | | |
| | | | CUGCUGGACAAGAA | | |
| | | | CGACUUCACCCGCG | | |
| | | | ACGCCGGCAUCAAC | | |
| | | | ACCGAUGAUAUUGU | | |
| | | | GGCCCUGGGCCUCG | | |
| | | | UGUACCAGUUUCAC | | |
| | | | CACCACCAUCACCA | | |
| | | | U | | |
| SEQ ID NO: | | 21 | 22 | 3 | 4 |

SEQ ID NO: 170 consists of from 5′ end to 3′ end, 5′ UTR SEQ
ID NO: 3, mRNA ORF SEQ ID NO: 22, and 3′ UTR SEQ ID NO: 4.

| | St_OmpC_ | METPAQLLFLLLLWL | AUGGAAACCCCUGC | GGGAAA | UGAUAA |
|---|---|---|---|---|---|
| | NGM_nIg | PDTTGAEIYNKDGNK | UCAGCUGCUGUUCC | UAAGAG | UAGGCU |
| | k | LDLFGKVDGLHYFSD | UGCUGCUGCUGUGG | AGAAAA | GGAGCC |
| | | DKGSDGDQTYMRIG | CUGCCUGAUACAAC | GAAGAG | UCGGUG |
| | | FKGETQVNDQLTGY | AGGCGCCGAGAUCU | UAAGAA | GCCAUG |
| | | GQWEYQIQGNQAEG | ACAACAAGGACGGC | GAAAUA | CUUCUU |
| | | SNDAWTRVAFAGLK | AACAAGCUGGACCU | UAAGAG | GCCCCU |
| | | FADAGSFDYGRNYG | GUUCGGCAAGGUGG | CCACC | UGGGCC |
| | | VTYDVTSWTDVLPEF | ACGGCCUGCACUAC | | UCCCCC |
| | | GGDTYGADNFMQQR | UUCAGCGACGAUAA | | CAGCCC |
| | | GNGYATYRNTDFFG | GGGCUCCGACGGCG | | CUCCUC |
| | | LVDGLDFALQYQGK | ACCAGACCUACAUG | | CCCUUC |
| | | NGAVSGENTNGRSLL | AGAAUCGGCUUCAA | | CUGCAC |
| | | NQNGDGYGGSLTYAI | GGGCGAGACACAAG | | CCGUAC |
| | | GEGFSVGGAITTSKR | UGAACGACCAGCUG | | CCCCGU |
| | | TADQNNAANARLYG | ACAGGCUACGGCCA | | GGUCUU |
| | | NGDRATVYTGGLKY | GUGGGAGUAUCAGA | | UGAAUA |
| | | DANNIYLAAQYSQTY | UCCAGGGCAAUCAG | | AAGUCU |
| | | NAARFGTSNGANPST | GCCGAGGGCAGCAA | | GAGUGG |
| | | SYGFANKAQNFEVV | CGACGCAUGGACCA | | GCGGC |
| | | AQYQFDFGLRPSVAY | GAGUGGCUUUUGCC | | |
| | | LQSKGKDISNGYGAS | GGCCUGAAGUUUGC | | |
| | | YGDQDIVKYVDVGA | CGAUGCCGGCAGCU | | |
| | | TYYFNKNMSTYVDY | UUGACUACGGCAGA | | |
| | | KINLLDKNDFTRDAG | AAUUACGGCGUGAC | | |
| | | INTDDIVALGLVYQF | CUACGACGUGACCU | | |
| | | | CCUGGACAGAUGUG | | |
| | | | CUGCCUGAGUUUGG | | |
| | | | CGGCGAUACCUACG | | |
| | | | GCGCCGACAACUUC | | |
| | | | AUGCAGCAGAGAGG | | |
| | | | CAACGGCUACGCCA | | |
| | | | CCUACCGGAACACC | | |
| | | | GAUUUCUUCGGCCU | | |
| | | | GGUGGAUGGCCUGG | | |
| | | | AUUUCGCCCUGCAG | | |
| | | | UACCAGGGCAAGAA | | |
| | | | UGGCGCUGUGUCCG | | |
| | | | GCGAGAACACCAAC | | |
| | | | GGCAGAAGCCUGCU | | |
| | | | GAACCAGAACGGCG | | |
| | | | ACGGAUAUGGCGGC | | |
| | | | AGCCUGACAUAUGC | | |
| | | | CAUCGGCGAGGGCU | | |
| | | | UUUCUGUCGGCGGA | | |
| | | | GCCAUCACCACCAG | | |
| | | | CAAGAGAACAGCCG | | |
| | | | ACCAGAACAACGCC | | |
| | | | GCCAACGCCAGACU | | |
| | | | GUACGGCAACGGCG | | |
| | | | AUAGAGCCACAGUG | | |
| | | | UAUACCGGCGGACU | | |
| | | | GAAGUACGACGCCA | | |
| | | | ACAACAUCUACCUG | | |
| | | | GCCGCUCAGUACAG | | |
| | | | CCAGACAUACAACG | | |
| | | | CCGCCAGAUUCGGC | | |
| | | | ACCAGCAACGGCGC | | |
| | | | AAAUCCCAGCACAA | | |
| | | | GCUACGGCUUCGCC | | |
| | | | AACAAGGCCCAGAA | | |
| | | | CUUUGAGGUGGUGG | | |
| | | | CCCAGUACCAGUUC | | |
| | | | GACUUCGGACUGAG | | |
| | | | GCCUAGCGUGGCCU | | |
| | | | ACCUGCAGAGCAAG | | |
| | | | GGCAAAGACAUCAG | | |
| | | | CAACGGAUACGGCG | | |
| | | | CCAGCUACGGCGAU | | |
| | | | CAGGACAUCGUGAA | | |
| | | | AUACGUGGACGUGG | | |
| | | | GCGCCACCUAUUAC | | |
| | | | UUCAACAAGAACAU | | |
| | | | GAGCACCUACGUGG | | |
| | | | ACUACAAGAUCAAC | | |
| | | | CUGCUGGACAAGAA | | |
| | | | CGACUUCACCCGCG | | |
| | | | ACGCCGGCAUCAAC | | |
| | | | ACCGAUGAUAUUGU | | |
| | | | GGCCCUGGGCCUCG | | |
| | | | UGUACCAGUUU | | |
| SEQ ID NO: | | 23 | 24 | 3 | 4 |

SEQ ID NO: 171 consists of from 5′ end to 3′ end, 5′ UTR SEQ
ID NO: 3, mRNA ORF SEQ ID NO: 24, and 3′ UTR SEQ ID NO: 4.

| | St_SseB_c | MIPMSETKNELEILLE | AUGAUCCCCAUGAG | GGGAAA | UGAUAA |
|---|---|---|---|---|---|
| | His | KAATEPAHRSAFFRT | CGAGACAAAGAACG | UAAGAG | UAGGCU |
| | | LLESTVWVPGSAAEG | AGCUGGAAAUCCUG | AGAAAA | GGAGCC |
| | | EAIVEDSALDLQHWE | CUCGAGAAGGCCGC | GAAGAG | UCGGUG |
| | | KEDGTTVIPFFTSLEA | CACAGAGCCUGCUC | UAAGAA | GCCAUG |
| | | LQQAVEDEQAFVVM | ACAGAAGCGCUUUC | GAAAUA | CUUCUU |
| | | PARTLFEMTLGETLF | UUCAGAACCCUGCU | UAAGAG | GCCCCU |
| | | LNAKLPTGKEFMPRE | GGAAAGCACCGUGU | CCACC | UGGGCC |
| | | ISLLLAEEGSPLSTQE | GGGUGCCAGGAUCU | | UCCCCC |
| | | VLEGGESLILSEVAEP | GCUGCUGAAGGCGA | | CAGCCC |
| | | PSQMIDSLTTLFKTIK | AGCCAUCGUGGAAG | | CUCCUC |
| | | PVKRAFLCAIKEHAD | AUAGCGCCCUGGAU | | CCCUUC |
| | | AQPNLLIGIEADGEIE | CUGCAGCACUGGGA | | CUGCAC |
| | | EIIHAAGNVATDTLP | GAAAGAGGACGGAA | | CCGUAC |
| | | GDEPIDICQVRKGAQ | CCACAGUCAUCCCA | | CCCCGU |
| | | GISHFITEHIAPFYERR | UUCUUCACCAGCCU | | GGUCUU |
| | | WGGFLRDFKQNRIIH | GGAAGCCCUGCAGC | | UGAAUA |
| | | HHHHH | AGGCUGUGGAAGAU | | AAGUCU |
| | | | GAGCAGGCCUUCGU | | GAGUGG |
| | | | GGUCAUGCCCGCCA | | GCGGC |
| | | | GAACACUGUUCGAG | | |
| | | | AUGACCCUGGGCGA | | |
| | | | GACACUGUUCCUGA | | |
| | | | ACGCCAAACUGCCC | | |
| | | | ACCGGCAAAGAAUU | | |
| | | | CAUGCCCAGAGAGA | | |
| | | | UCUCCCUGCUGCUG | | |
| | | | GCCGAGGAAGGAUC | | |
| | | | UCCUCUGAGCACAC | | |
| | | | AAGAGGUGCUGGAA | | |
| | | | GGCGGCGAGAGCCU | | |
| | | | GAUUCUGUCUGAAG | | |
| | | | UGGCCGAGCCUCCU | | |
| | | | AGCCAGAUGAUCGA | | |
| | | | CAGCCUGACCACAC | | |
| | | | UGUUCAAGACCAUC | | |
| | | | AAGCCCGUGAAGCG | | |
| | | | GGCCUUCCUGUGCG | | |
| | | | CCAUCAAAGAACAC | | |
| | | | GCUGACGCCCAGCC | | |
| | | | UAACCUGCUGAUCG | | |
| | | | GAAUUGAGGCCGAC | | |
| | | | GGCGAGAUCGAGGA | | |
| | | | AAUCAUCCACGCCG | | |
| | | | CUGGAAACGUGGCC | | |
| | | | ACCGAUACACUGCC | | |
| | | | UGGCGACGAGCCUA | | |
| | | | UCGACAUCUGCCAA | | |
| | | | GUUCGGAAAGGCGC | | |
| | | | CCAGGGAAUCAGCC | | |
| | | | ACUUCAUCACCGAA | | |
| | | | CACAUUGCCCCAUU | | |
| | | | CUACGAGCGGAGAU | | |
| | | | GGGGCGGCUUCCUG | | |
| | | | AGAGACUUCAAGCA | | |
| | | | GAACCGGAUCAUCC | | |
| | | | ACCACCACCAUCAC | | |
| | | | CAC | | |
| SEQ ID NO: | | 25 | 26 | 3 | 4 |

SEQ ID NO: 172 consists of from 5′ end to 3′ end, 5′ UTR SEQ
ID NO: 3, mRNA ORF SEQ ID NO: 26, and 3′ UTR SEQ ID NO: 4.

| | St_SseB | MIPMSETKNELEILLE | AUGAUCCCCAUGAG | GGGAAA | UGAUAA |
|---|---|---|---|---|---|
| | | KAATEPAHRSAFFRT | CGAGACAAAGAACG | UAAGAG | UAGGCU |
| | | LLESTVWVPGSAAEG | AGCUGGAAAUCCUG | AGAAAA | GGAGCC |
| | | EAIVEDSALDLQHWE | CUCGAGAAGGCCGC | GAAGAG | UCGGUG |
| | | KEDGTTVIPFFTSLEA | CACAGAGCCUGCUC | UAAGAA | GCCAUG |
| | | LQQAVEDEQAFVVM | ACAGAAGCGCUUUC | GAAAUA | CUUCUU |
| | | PARTLFEMTLGETLF | UUCAGAACCCUGCU | UAAGAG | GCCCCU |
| | | LNAKLPTGKEFMPRE | GGAAAGCACCGUGU | CCACC | UGGGCC |
| | | ISLLLAEEGSPLSTQE | GGGUGCCAGGAUCU | | UCCCCC |
| | | VLEGGESLILSEVAEP | GCUGCUGAAGGCGA | | CAGCCC |
| | | PSQMIDSLTTLFKTIK | AGCCAUCGUGGAAG | | CUCCUC |
| | | PVKRAFLCAIKEHAD | AUAGCGCCCUGGAU | | CCCUUC |
| | | AQPNLLIGIEADGEIE | CUGCAGCACUGGGA | | CUGCAC |
| | | EIIHAAGNVATDTLP | GAAAGAGGACGGAA | | CCGUAC |
| | | GDEPIDICQVRKGAQ | CCACAGUCAUCCCA | | CCCCGU |
| | | GISHFITEHIAPFYERR | UUCUUCACCAGCCU | | GGUCUU |
| | | WGGFLRDFKQNRII | GGAAGCCCUGCAGC | | UGAAUA |
| | | | AGGCUGUGGAAGAU | | AAGUCU |
| | | | GAGCAGGCCUUCGU | | GAGUGG |
| | | | GGUCAUGCCCGCCA | | GCGGC |
| | | | GAACACUGUUCGAG | | |
| | | | AUGACCCUGGGCGA | | |
| | | | GACACUGUUCCUGA | | |
| | | | ACGCCAAACUGCCC | | |
| | | | ACCGGCAAAGAAUU | | |
| | | | CAUGCCCAGAGAGA | | |
| | | | UCUCCCUGCUGCUG | | |
| | | | GCCGAGGAAGGAUC | | |
| | | | UCCUCUGAGCACAC | | |
| | | | AAGAGGUGCUGGAA | | |
| | | | GGCGGCGAGAGCCU | | |
| | | | GAUUCUGUCUGAAG | | |
| | | | UGGCCGAGCCUCCU | | |
| | | | AGCCAGAUGAUCGA | | |
| | | | CAGCCUGACCACAC | | |
| | | | UGUUCAAGACCAUC | | |
| | | | AAGCCCGUGAAGCG | | |
| | | | GGCCUUCCUGUGCG | | |
| | | | CCAUCAAAGAACAC | | |
| | | | GCUGACGCCCAGCC | | |
| | | | UAACCUGCUGAUCG | | |
| | | | GAAUUGAGGCCGAC | | |
| | | | GGCGAGAUCGAGGA | | |
| | | | AAUCAUCCACGCCG | | |
| | | | CUGGAAACGUGGCC | | |
| | | | ACCGAUACACUGCC | | |
| | | | UGGCGACGAGCCUA | | |
| | | | UCGACAUCUGCCAA | | |
| | | | GUUCGGAAAGGCGC | | |
| | | | CCAGGGAAUCAGCC | | |
| | | | ACUUCAUCACCGAA | | |
| | | | CACAUUGCCCCAUU | | |
| | | | CUACGAGCGGAGAU | | |
| | | | GGGGCGGCUUCCUG | | |
| | | | AGAGACUUCAAGCA | | |
| | | | GAACCGGAUCAUC | | |
| SEQ ID NO: | | 27 | 28 | 3 | 4 |

SEQ ID NO: 173 consists of from 5′ end to 3′ end, 5′ UTR SEQ
ID NO: 3, mRNA ORF SEQ ID NO: 28, and 3′ UTR SEQ ID NO: 4.

| | St_SseB_n | METPAQLLFLLLLWL | AUGGAAACCCCUGC | GGGAAA | UGAUAA |
|---|---|---|---|---|---|
| | IgK_cHis | PDTTGIPMSETKNELE | UCAGCUGCUGUUCC | UAAGAG | UAGGCU |
| | | ILLEKAATEPAHRSAF | UGCUGCUGCUGUGG | AGAAAA | GGAGCC |
| | | FRTLLESTVWVPGSA | CUGCCUGAUACAAC | GAAGAG | UCGGUG |
| | | AEGEAIVEDSALDLQ | AGGCAUCCCCAUGA | UAAGAA | GCCAUG |
| | | HWEKEDGTTVIPFFT | GCGAGACAAAGAAC | GAAAUA | CUUCUU |
| | | SLEALQQAVEDEQAF | GAGCUGGAAAUCCU | UAAGAG | GCCCCU |
| | | VVMPARTLFEMTLG | GCUCGAGAAGGCCG | CCACC | UGGGCC |
| | | ETLFLNAKLPTGKEF | CCACAGAGCCUGCU | | UCCCCC |
| | | MPREISLLLAEEGSPL | CACAGAAGCGCUUU | | CAGCCC |
| | | STQEVLEGGESLILSE | CUUCAGAACCCUGC | | CUCCUC |
| | | VAEPPSQMIDSLTTLF | UGGAAAGCACCGUG | | CCCUUC |
| | | KTIKPVKRAFLCAIKE | UGGGUGCCAGGAUC | | CUGCAC |
| | | HADAQPNLLIGIEAD | UGCUGCUGAAGGCG | | CCGUAC |
| | | GEIEEIIHAAGNVATD | AAGCCAUCGUGGAA | | CCCCGU |
| | | TLPGDEPIDICQVRKG | GAUAGCGCCCUGGA | | GGUCUU |
| | | AQGISHFITEHIAPFY | UCUGCAGCACUGGG | | UGAAUA |
| | | ERRWGGFLRDFKQN | AGAAAGAGGACGGA | | AAGUCU |
| | | RIIHHHHHH | ACCACAGUCAUCCC | | GAGUGG |
| | | | AUUCUUCACCAGCC | | GCGGC |
| | | | UGGAAGCCCUGCAG | | |
| | | | CAGGCUGUGGAAGA | | |
| | | | UGAGCAGGCCUUCG | | |
| | | | UGGUCAUGCCCGCC | | |
| | | | AGAACACUGUUCGA | | |
| | | | GAUGACCCUGGGCG | | |
| | | | AGACACUGUUCCUG | | |
| | | | AACGCCAAACUGCC | | |
| | | | CACCGGCAAAGAAU | | |
| | | | UCAUGCCCAGAGAG | | |
| | | | AUCUCCCUGCUGCU | | |
| | | | GGCCGAGGAAGGAU | | |
| | | | CUCCUCUGAGCACA | | |
| | | | CAAGAGGUGCUGGA | | |
| | | | AGGCGGCGAGAGCC | | |
| | | | UGAUUCUGUCUGAA | | |
| | | | GUGGCCGAGCCUCC | | |
| | | | UAGCCAGAUGAUCG | | |
| | | | ACAGCCUGACCACA | | |
| | | | CUGUUCAAGACCAU | | |
| | | | CAAGCCCGUGAAGC | | |
| | | | GGGCCUUCCUGUGC | | |
| | | | GCCAUCAAAGAACA | | |
| | | | CGCUGACGCCCAGC | | |
| | | | CUAACCUGCUGAUC | | |
| | | | GGAAUUGAGGCCGA | | |
| | | | CGGCGAGAUCGAGG | | |
| | | | AAAUCAUCCACGCC | | |
| | | | GCUGGAAACGUGGC | | |
| | | | CACCGAUACACUGC | | |
| | | | CUGGCGACGAGCCU | | |
| | | | AUCGACAUCUGCCA | | |
| | | | AGUUCGGAAAGGCG | | |
| | | | CCCAGGGAAUCAGC | | |
| | | | CACUUCAUCACCGA | | |
| | | | ACACAUUGCCCCAU | | |
| | | | UCUACGAGCGGAGA | | |
| | | | UGGGGCGGCUUCCU | | |
| | | | GAGAGACUUCAAGC | | |
| | | | AGAACCGGAUCAUC | | |
| | | | CACCACCACCAUCA | | |
| | | | CCAC | | |
| SEQ ID NO: | | 29 | 30 | 3 | 4 |

SEQ ID NO: 174 consists of from 5′ end to 3′ end, 5′ UTR SEQ
ID NO: 3, mRNA ORF SEQ ID NO: 30, and 3′ UTR SEQ ID NO: 4.

| | St_SseB_n | METPAQLLFLLLLWL | AUGGAAACCCCUGC | GGGAAA | UGAUAA |
|---|---|---|---|---|---|
| | IgK | PDTTGIPMSETKNELE | UCAGCUGCUGUUCC | UAAGAG | UAGGCU |
| | | ILLEKAATEPAHRSAF | UGCUGCUGCUGUGG | AGAAAA | GGAGCC |
| | | FRTLLESTVWVPGSA | CUGCCUGAUACAAC | GAAGAG | UCGGUG |
| | | AEGEAIVEDSALDLQ | AGGCAUCCCCAUGA | UAAGAA | GCCAUG |
| | | HWEKEDGTTVIPFFT | GCGAGACAAAGAAC | GAAAUA | CUUCUU |
| | | SLEALQQAVEDEQAF | GAGCUGGAAAUCCU | UAAGAG | GCCCCU |
| | | VVMPARTLFEMTLG | GCUCGAGAAGGCCG | CCACC | UGGGCC |
| | | ETLFLNAKLPTGKEF | CCACAGAGCCUGCU | | UCCCCC |
| | | MPREISLLLAEEGSPL | CACAGAAGCGCUUU | | CAGCCC |
| | | STQEVLEGGESLILSE | CUUCAGAACCCUGC | | CUCCUC |
| | | VAEPPSQMIDSLTTLF | UGGAAAGCACCGUG | | CCCUUC |
| | | KTIKPVKRAFLCAIKE | UGGGUGCCAGGAUC | | CUGCAC |
| | | HADAQPNLLIGIEAD | UGCUGCUGAAGGCG | | CCGUAC |
| | | GEIEEIIHAAGNVATD | AAGCCAUCGUGGAA | | CCCCGU |
| | | TLPGDEPIDICQVRKG | GAUAGCGCCCUGGA | | GGUCUU |
| | | AQGISHFITEHIAPFY | UCUGCAGCACUGGG | | UGAAUA |
| | | ERRWGGFLRDFKQN | AGAAAGAGGACGGA | | AAGUCU |
| | | RII | ACCACAGUCAUCCC | | GAGUGG |
| | | | AUUCUUCACCAGCC | | GCGGC |
| | | | UGGAAGCCCUGCAG | | |
| | | | CAGGCUGUGGAAGA | | |
| | | | UGAGCAGGCCUUCG | | |
| | | | UGGUCAUGCCCGCC | | |
| | | | AGAACACUGUUCGA | | |
| | | | GAUGACCCUGGGCG | | |
| | | | AGACACUGUUCCUG | | |
| | | | AACGCCAAACUGCC | | |
| | | | CACCGGCAAAGAAU | | |
| | | | UCAUGCCCAGAGAG | | |
| | | | AUCUCCCUGCUGCU | | |
| | | | GGCCGAGGAAGGAU | | |
| | | | CUCCUCUGAGCACA | | |
| | | | CAAGAGGUGCUGGA | | |
| | | | AGGCGGCGAGAGCC | | |
| | | | UGAUUCUGUCUGAA | | |
| | | | GUGGCCGAGCCUCC | | |
| | | | UAGCCAGAUGAUCG | | |
| | | | ACAGCCUGACCACA | | |
| | | | CUGUUCAAGACCAU | | |
| | | | CAAGCCCGUGAAGC | | |
| | | | GGGCCUUCCUGUGC | | |
| | | | GCCAUCAAAGAACA | | |
| | | | CGCUGACGCCCAGC | | |
| | | | CUAACCUGCUGAUC | | |
| | | | GGAAUUGAGGCCGA | | |
| | | | CGGCGAGAUCGAGG | | |
| | | | AAAUCAUCCACGCC | | |
| | | | GCUGGAAACGUGGC | | |
| | | | CACCGAUACACUGC | | |
| | | | CUGGCGACGAGCCU | | |
| | | | AUCGACAUCUGCCA | | |
| | | | AGUUCGGAAAGGCG | | |
| | | | CCCAGGGAAUCAGC | | |
| | | | CACUUCAUCACCGA | | |
| | | | ACACAUUGCCCCAU | | |
| | | | UCUACGAGCGGAGA | | |
| | | | UGGGGCGGCUUCCU | | |
| | | | GAGAGACUUCAAGC | | |
| | | | AGAACCGGAUCAUC | | |
| SEQ ID NO: | | 31 | 32 | 3 | 4 |

SEQ ID NO: 175 consists of from 5′ end to 3′ end, 5′ UTR SEQ
ID NO: 3, mRNA ORF SEQ ID NO: 32, and 3′ UTR SEQ ID NO: 4.

| | St_FliC_c | MAQVINTNSLSLLTQ | AUGGCCCAAGUGAU | GGGAAA | UGAUAA |
|---|---|---|---|---|---|
| | His | NNLNKSQSALGTAIE | CAACACCAACAGCC | UAAGAG | UAGGCU |
| | | RLSSGLRINSAKDDA | UGAGCCUGCUGACC | AGAAAA | GGAGCC |
| | | AGQAIANRFTANIKG | CAGAACAACCUGAA | GAAGAG | UCGGUG |
| | | LTQASRNANDGISIA | CAAGAGCCAGAGCG | UAAGAA | GCCAUG |
| | | QTTEGALNEINNNLQ | CCCUGGGCACAGCC | GAAAUA | CUUCUU |
| | | RVRELAVQSANGTNS | AUCGAAAGACUGUC | UAAGAG | GCCCCU |
| | | QSDLDSIQAEITQRLN | UAGCGGCCUGCGGA | CCACC | UGGGCC |
| | | EIDRVSGQTQFNGVK | UCAACAGCGCCAAA | | UCCCCC |
| | | VLAQDNTLTIQVGAN | GAUGAUGCUGCCGG | | CAGCCC |
| | | DGETIDIDLKEISSKT | ACAGGCCAUUGCCA | | CUCCUC |
| | | LGLDKLNVQDAYTP | ACAGAUUCACCGCC | | CCCUUC |
| | | KETAVTVDKTTYKN | AACAUCAAGGGCCU | | CUGCAC |
| | | GTDPITAQSNTDIQTA | GACACAGGCCAGCA | | CCGUAC |
| | | IGGGATGVTGADIKF | GAAACGCCAACGAC | | CCCCGU |
| | | KDGQYYLDVKGGAS | GGCAUCUCUAUCGC | | GGUCUU |
| | | AGVYKATYDETTKK | CCAGACAACAGAAG | | UGAAUA |
| | | VNIDTTDKTPLATAE | GCGCCCUGAACGAG | | AAGUCU |
| | | ATAIRGTATITHNQIA | AUCAACAACAACCU | | GAGUGG |
| | | EVTKEGVDTTTVAA | GCAGAGAGUGCGCG | | GCGGC |
| | | QLAAAGVTGADKDN | AGCUGGCCGUGCAA | | |
| | | TSLVKLSFEDKNGKV | UCUGCCAAUGGCAC | | |
| | | IDGGYAVKMGDDFY | AAACAGCCAGUCCG | | |
| | | AATYDEKTGAITAKT | ACCUGGAUUCCAUC | | |
| | | TTYTDGTGVAQTGA | CAGGCCGAGAUCAC | | |
| | | VKFGGANGKSEVVT | CCAGCGGCUGAAUG | | |
| | | ATDGKTYLASDLDK | AGAUCGACAGAGUG | | |
| | | HNFRTGGELKEVNTD | UCCGGCCAGACACA | | |
| | | KTENPLQKIDAALAQ | GUUCAACGGCGUGA | | |
| | | VDTLRSDLGAVQNRF | AAGUGCUGGCCCAG | | |
| | | NSAITNLGNTVNNLS | GACAACACCCUGAC | | |
| | | SARSRIEDSDYATEVS | CAUCCAAGUGGGAG | | |
| | | NMSRAQILQQAGTSV | CCAACGAUGGCGAG | | |
| | | LAQANQVPQNVLSLL | ACAAUCGACAUCGA | | |
| | | RHHHHHH | CCUGAAAGAGAUCU | | |
| | | | CCAGCAAGACCCUG | | |
| | | | GGCCUCGACAAGCU | | |
| | | | GAACGUGCAGGAUG | | |
| | | | CCUACACACCCAAA | | |
| | | | GAAACCGCCGUGAC | | |
| | | | CGUGGACAAGACCA | | |
| | | | CCUACAAGAACGGC | | |
| | | | ACAGACCCCAUCAC | | |
| | | | AGCCCAGAGCAACA | | |
| | | | CCGAUAUCCAGACC | | |
| | | | GCCAUUGGAGGCGG | | |
| | | | AGCUACAGGUGUUA | | |
| | | | CAGGCGCCGACAUC | | |
| | | | AAGUUCAAGGACGG | | |
| | | | CCAGUACUACCUGG | | |
| | | | ACGUGAAAGGCGGA | | |
| | | | GCAUCUGCCGGCGU | | |
| | | | GUACAAGGCCACAU | | |
| | | | ACGACGAAACCACC | | |
| | | | AAGAAAGUGAACAU | | |
| | | | CGACACCACCGACA | | |
| | | | AGACCCCUCUGGCC | | |
| | | | ACAGCUGAAGCCAC | | |
| | | | AGCCAUUAGAGGCA | | |
| | | | CCGCCACAAUCACC | | |
| | | | CACAACCAGAUCGC | | |
| | | | CGAAGUGACCAAAG | | |
| | | | AAGGCGUGGACACC | | |
| | | | ACAACCGUGGCUGC | | |
| | | | UCAACUUGCUGCUG | | |
| | | | CUGGCGUUACCGGC | | |
| | | | GCUGACAAGGAUAA | | |
| | | | UACCAGCCUGGUCA | | |
| | | | AGCUGAGCUUCGAG | | |
| | | | GACAAGAAUGGCAA | | |
| | | | AGUGAUCGACGGCG | | |
| | | | GCUACGCCGUGAAG | | |
| | | | AUGGGCGACGAUUU | | |
| | | | CUACGCCGCCACCU | | |
| | | | ACGAUGAGAAGACC | | |
| | | | GGCGCCAUUACCGC | | |
| | | | CAAGACCACAACCU | | |
| | | | ACACAGAUGGCACA | | |
| | | | GGCGUGGCACAGAC | | |
| | | | AGGGGCCGUGAAAU | | |
| | | | UUGGAGGCGCCAAC | | |
| | | | GGCAAGAGCGAGGU | | |
| | | | CGUGACAGCUACCG | | |
| | | | ACGGCAAGACAUAC | | |
| | | | CUGGCCAGCGAUCU | | |
| | | | GGACAAGCACAACU | | |
| | | | UCAGAACAGGCGGC | | |
| | | | GAGCUGAAAGAAGU | | |
| | | | GAACACAGACAAGA | | |
| | | | CCGAGAAUCCGCUG | | |
| | | | CAGAAGAUCGACGC | | |
| | | | UGCUCUGGCACAAG | | |
| | | | UGGACACCCUGAGA | | |
| | | | AGUGAUCUGGGCGC | | |
| | | | CGUGCAGAACAGGU | | |
| | | | UCAACUCCGCCAUC | | |
| | | | ACCAACCUGGGCAA | | |
| | | | CACCGUGAACAAUC | | |
| | | | UGAGCAGCGCCAGA | | |
| | | | AGCCGGAUCGAGGA | | |
| | | | CAGCGAUUAUGCCA | | |
| | | | CCGAGGUGUCCAAC | | |
| | | | AUGAGCAGAGCCCA | | |
| | | | GAUUCUGCAGCAGG | | |
| | | | CCGGCACAUCUGUU | | |
| | | | CUGGCUCAGGCAAA | | |
| | | | UCAGGUGCCCCAGA | | |
| | | | ACGUGCUGUCCCUG | | |
| | | | CUGAGACACCACCA | | |
| | | | CCAUCACCAU | | |
| SEQ ID NO: | | 33 | 34 | 3 | 4 |

SEQ ID NO: 176 consists of from 5′ end to 3′ end, 5′ UTR SEQ
ID NO: 3, mRNA ORF SEQ ID NO: 34, and 3′ UTR SEQ ID NO: 4.

| | St_FliC | MAQVINTNSLSLLTQ | AUGGCCCAAGUGAU | GGGAAA | UGAUAA |
|---|---|---|---|---|---|
| | | NNLNKSQSALGTAIE | CAACACCAACAGCC | UAAGAG | UAGGCU |
| | | RLSSGLRINSAKDDA | UGAGCCUGCUGACC | AGAAAA | GGAGCC |
| | | AGQAIANRFTANIKG | CAGAACAACCUGAA | GAAGAG | UCGGUG |
| | | LTQASRNANDGISIA | CAAGAGCCAGAGCG | UAAGAA | GCCAUG |
| | | QTTEGALNEINNNLQ | CCCUGGGCACAGCC | GAAAUA | CUUCUU |
| | | RVRELAVQSANGTNS | AUCGAAAGACUGUC | UAAGAG | GCCCCU |
| | | QSDLDSIQAEITQRLN | UAGCGGCCUGCGGA | CCACC | UGGGCC |
| | | EIDRVSGQTQFNGVK | UCAACAGCGCCAAA | | UCCCCC |
| | | VLAQDNTLTIQVGAN | GAUGAUGCUGCCGG | | CAGCCC |
| | | DGETIDIDLKEISSKT | ACAGGCCAUUGCCA | | CUCCUC |
| | | LGLDKLNVQDAYTP | ACAGAUUCACCGCC | | CCCUUC |
| | | KETAVTVDKTTYKN | AACAUCAAGGGCCU | | CUGCAC |
| | | GTDPITAQSNTDIQTA | GACACAGGCCAGCA | | CCGUAC |
| | | IGGGATGVTGADIKF | GAAACGCCAACGAC | | CCCCGU |
| | | KDGQYYLDVKGGAS | GGCAUCUCUAUCGC | | GGUCUU |
| | | AGVYKATYDETTKK | CCAGACAACAGAAG | | UGAAUA |
| | | VNIDTTDKTPLATAE | GCGCCCUGAACGAG | | AAGUCU |
| | | ATAIRGTATITHNQIA | AUCAACAACAACCU | | GAGUGG |
| | | EVTKEGVDTTTVAA | GCAGAGAGUGCGCG | | GCGGC |
| | | QLAAAGVTGADKDN | AGCUGGCCGUGCAA | | |
| | | TSLVKLSFEDKNGKV | UCUGCCAAUGGCAC | | |
| | | IDGGYAVKMGDDFY | AAACAGCCAGUCCG | | |
| | | AATYDEKTGAITAKT | ACCUGGAUUCCAUC | | |
| | | TTYTDGTGVAQTGA | CAGGCCGAGAUCAC | | |
| | | VKFGGANGKSEVVT | CCAGCGGCUGAAUG | | |
| | | ATDGKTYLASDLDK | AGAUCGACAGAGUG | | |
| | | HNFRTGGELKEVNTD | UCCGGCCAGACACA | | |
| | | KTENPLQKIDAALAQ | GUUCAACGGCGUGA | | |
| | | VDTLRSDLGAVQNRF | AAGUGCUGGCCCAG | | |
| | | NSAITNLGNTVNNLS | GACAACACCCUGAC | | |
| | | SARSRIEDSDYATEVS | CAUCCAAGUGGGAG | | |
| | | NMSRAQILQQAGTSV | CCAACGAUGGCGAG | | |
| | | LAQANQVPQNVLSLL | ACAAUCGACAUCGA | | |
| | | R | CCUGAAAGAGAUCU | | |
| | | | CCAGCAAGACCCUG | | |
| | | | GGCCUCGACAAGCU | | |
| | | | GAACGUGCAGGAUG | | |
| | | | CCUACACACCCAAA | | |
| | | | GAAACCGCCGUGAC | | |
| | | | CGUGGACAAGACCA | | |
| | | | CCUACAAGAACGGC | | |
| | | | ACAGACCCCAUCAC | | |
| | | | AGCCCAGAGCAACA | | |
| | | | CCGAUAUCCAGACC | | |
| | | | GCCAUUGGAGGCGG | | |
| | | | AGCUACAGGUGUUA | | |
| | | | CAGGCGCCGACAUC | | |
| | | | AAGUUCAAGGACGG | | |
| | | | CCAGUACUACCUGG | | |
| | | | ACGUGAAAGGCGGA | | |
| | | | GCAUCUGCCGGCGU | | |
| | | | GUACAAGGCCACAU | | |
| | | | ACGACGAAACCACC | | |
| | | | AAGAAAGUGAACAU | | |
| | | | CGACACCACCGACA | | |
| | | | AGACCCCUCUGGCC | | |
| | | | ACAGCUGAAGCCAC | | |
| | | | AGCCAUUAGAGGCA | | |
| | | | CCGCCACAAUCACC | | |
| | | | CACAACCAGAUCGC | | |
| | | | CGAAGUGACCAAAG | | |
| | | | AAGGCGUGGACACC | | |
| | | | ACAACCGUGGCUGC | | |
| | | | UCAACUUGCUGCUG | | |
| | | | CUGGCGUUACCGGC | | |
| | | | GCUGACAAGGAUAA | | |
| | | | UACCAGCCUGGUCA | | |
| | | | AGCUGAGCUUCGAG | | |
| | | | GACAAGAAUGGCAA | | |
| | | | AGUGAUCGACGGCG | | |
| | | | GCUACGCCGUGAAG | | |
| | | | AUGGGCGACGAUUU | | |
| | | | CUACGCCGCCACCU | | |
| | | | ACGAUGAGAAGACC | | |
| | | | GGCGCCAUUACCGC | | |
| | | | CAAGACCACAACCU | | |
| | | | ACACAGAUGGCACA | | |
| | | | GGCGUGGCACAGAC | | |
| | | | AGGGGCCGUGAAAU | | |
| | | | UUGGAGGCGCCAAC | | |
| | | | GGCAAGAGCGAGGU | | |
| | | | CGUGACAGCUACCG | | |
| | | | ACGGCAAGACAUAC | | |
| | | | CUGGCCAGCGAUCU | | |
| | | | GGACAAGCACAACU | | |
| | | | UCAGAACAGGCGGC | | |
| | | | GAGCUGAAAGAAGU | | |
| | | | GAACACAGACAAGA | | |
| | | | CCGAGAAUCCGCUG | | |
| | | | CAGAAGAUCGACGC | | |
| | | | UGCUCUGGCACAAG | | |
| | | | UGGACACCCUGAGA | | |
| | | | AGUGAUCUGGGCGC | | |
| | | | CGUGCAGAACAGGU | | |
| | | | UCAACUCCGCCAUC | | |
| | | | ACCAACCUGGGCAA | | |
| | | | CACCGUGAACAAUC | | |
| | | | UGAGCAGCGCCAGA | | |
| | | | AGCCGGAUCGAGGA | | |
| | | | CAGCGAUUAUGCCA | | |
| | | | CCGAGGUGUCCAAC | | |
| | | | AUGAGCAGAGCCCA | | |
| | | | GAUUCUGCAGCAGG | | |
| | | | CCGGCACAUCUGUU | | |
| | | | CUGGCUCAGGCAAA | | |
| | | | UCAGGUGCCCCAGA | | |
| | | | ACGUGCUGUCCCUG | | |
| | | | CUGAGA | | |
| SEQ ID NO: | | 35 | 36 | 3 | 4 |

SEQ ID NO: 177 consists of from 5′ end to 3′ end, 5′ UTR SEQ
ID NO: 3, mRNA ORF SEQ ID NO: 36, and 3′ UTR SEQ ID NO: 4.

| | St_FliC_nI | METPAQLLFLLLLWL | AUGGAAACCCCUGC | GGGAAA | UGAUAA |
|---|---|---|---|---|---|
| | gK_cHis | PDTTGAQVINTNSLS | UCAGCUGCUGUUCC | UAAGAG | UAGGCU |
| | | LLTQNNLNKSQSALG | UGCUGCUGCUGUGG | AGAAAA | GGAGCC |
| | | TAIERLSSGLRINSAK | CUGCCUGAUACAAC | GAAGAG | UCGGUG |
| | | DDAAGQAIANRFTA | AGGCGCCCAAGUGA | UAAGAA | GCCAUG |
| | | NIKGLTQASRNANDG | UCAACACCAACAGC | GAAAUA | CUUCUU |
| | | ISIAQTTEGALNEINN | CUGAGCCUGCUGAC | UAAGAG | GCCCCU |
| | | NLQRVRELAVQSAN | CCAGAACAACCUGA | CCACC | UGGGCC |
| | | GTNSQSDLDSIQAEIT | ACAAGAGCCAGAGC | | UCCCCC |
| | | QRLNEIDRVSGQTQF | GCCCUGGGCACAGC | | CAGCCC |
| | | NGVKVLAQDNTLTIQ | CAUCGAAAGACUGU | | CUCCUC |
| | | VGANDGETIDIDLKEI | CUAGCGGCCUGCGG | | CCCUUC |
| | | SSKTLGLDKLNVQDA | AUCAACAGCGCCAA | | CUGCAC |
| | | YTPKETAVTVDKTTY | AGAUGAUGCUGCCG | | CCGUAC |
| | | KNGTDPITAQSNTDI | GACAGGCCAUUGCC | | CCCCGU |
| | | QTAIGGGATGVTGA | AACAGAUUCACCGC | | GGUCUU |
| | | DIKFKDGQYYLDVK | CAACAUCAAGGGCC | | UGAAUA |
| | | GGASAGVYKATYDE | UGACACAGGCCAGC | | AAGUCU |
| | | TTKKVNIDTTDKTPL | AGAAACGCCAACGA | | GAGUGG |
| | | ATAEATAIRGTATITH | CGGCAUCUCUAUCG | | GCGGC |
| | | NQIAEVTKEGVDTTT | CCCAGACAACAGAA | | |
| | | VAAQLAAAGVTGAD | GGCGCCCUGAACGA | | |
| | | KDNTSLVKLSFEDKN | GAUCAACAACAACC | | |
| | | GKVIDGGYAVKMGD | UGCAGAGAGUGCGC | | |
| | | DFYAATYDEKTGAIT | GAGCUGGCCGUGCA | | |
| | | AKTTTYTDGTGVAQ | AUCUGCCAAUGGCA | | |
| | | TGAVKFGGANGKSE | CAAACAGCCAGUCC | | |
| | | VVTATDGKTYLASD | GACCUGGAUUCCAU | | |
| | | LDKHNFRTGGELKEV | CCAGGCCGAGAUCA | | |
| | | NTDKTENPLQKIDAA | CCCAGCGGCUGAAU | | |
| | | LAQVDTLRSDLGAV | GAGAUCGACAGAGU | | |
| | | QNRFNSAITNLGNTV | GUCCGGCCAGACAC | | |
| | | NNLSSARSRIEDSDY | AGUUCAACGGCGUG | | |
| | | ATEVSNMSRAQILQQ | AAAGUGCUGGCCCA | | |
| | | AGTSVLAQANQVPQ | GGACAACACCCUGA | | |
| | | NVLSLLRHHHHHH | CCAUCCAAGUGGGA | | |
| | | | GCCAACGAUGGCGA | | |
| | | | GACAAUCGACAUCG | | |
| | | | ACCUGAAAGAGAUC | | |
| | | | UCCAGCAAGACCCU | | |
| | | | GGGCCUCGACAAGC | | |
| | | | UGAACGUGCAGGAU | | |
| | | | GCCUACACACCCAA | | |
| | | | AGAAACCGCCGUGA | | |
| | | | CCGUGGACAAGACC | | |
| | | | ACCUACAAGAACGG | | |
| | | | CACAGACCCCAUCA | | |
| | | | CAGCCCAGAGCAAC | | |
| | | | ACCGAUAUCCAGAC | | |
| | | | CGCCAUUGGAGGCG | | |
| | | | GAGCUACAGGUGUU | | |
| | | | ACAGGCGCCGACAU | | |
| | | | CAAGUUCAAGGACG | | |
| | | | GCCAGUACUACCUG | | |
| | | | GACGUGAAAGGCGG | | |
| | | | AGCAUCUGCCGGCG | | |
| | | | UGUACAAGGCCACA | | |
| | | | UACGACGAAACCAC | | |
| | | | CAAGAAAGUGAACA | | |
| | | | UCGACACCACCGAC | | |
| | | | AAGACCCCUCUGGC | | |
| | | | CACAGCUGAAGCCA | | |
| | | | CAGCCAUUAGAGGC | | |
| | | | ACCGCCACAAUCAC | | |
| | | | CCACAACCAGAUCG | | |
| | | | CCGAAGUGACCAAA | | |
| | | | GAAGGCGUGGACAC | | |
| | | | CACAACCGUGGCUG | | |
| | | | CUCAACUUGCUGCU | | |
| | | | GCUGGCGUUACCGG | | |
| | | | CGCUGACAAGGAUA | | |
| | | | AUACCAGCCUGGUC | | |
| | | | AAGCUGAGCUUCGA | | |
| | | | GGACAAGAAUGGCA | | |
| | | | AAGUGAUCGACGGC | | |
| | | | GGCUACGCCGUGAA | | |
| | | | GAUGGGCGACGAUU | | |
| | | | UCUACGCCGCCACC | | |
| | | | UACGAUGAGAAGAC | | |
| | | | CGGCGCCAUUACCG | | |
| | | | CCAAGACCACAACC | | |
| | | | UACACAGAUGGCAC | | |
| | | | AGGCGUGGCACAGA | | |
| | | | CAGGGGCCGUGAAA | | |
| | | | UUUGGAGGCGCCAA | | |
| | | | CGGCAAGAGCGAGG | | |
| | | | UCGUGACAGCUACC | | |
| | | | GACGGCAAGACAUA | | |
| | | | CCUGGCCAGCGAUC | | |
| | | | UGGACAAGCACAAC | | |
| | | | UUCAGAACAGGCGG | | |
| | | | CGAGCUGAAAGAAG | | |
| | | | UGAACACAGACAAG | | |
| | | | ACCGAGAAUCCGCU | | |
| | | | GCAGAAGAUCGACG | | |
| | | | CUGCUCUGGCACAA | | |
| | | | GUGGACACCCUGAG | | |
| | | | AAGUGAUCUGGGCG | | |
| | | | CCGUGCAGAACAGG | | |
| | | | UUCAACUCCGCCAU | | |
| | | | CACCAACCUGGGCA | | |
| | | | ACACCGUGAACAAU | | |
| | | | CUGAGCAGCGCCAG | | |
| | | | AAGCCGGAUCGAGG | | |
| | | | ACAGCGAUUAUGCC | | |
| | | | ACCGAGGUGUCCAA | | |
| | | | CAUGAGCAGAGCCC | | |
| | | | AGAUUCUGCAGCAG | | |
| | | | GCCGGCACAUCUGU | | |
| | | | UCUGGCUCAGGCAA | | |
| | | | AUCAGGUGCCCCAG | | |
| | | | AACGUGCUGUCCCU | | |
| | | | GCUGAGACACCACC | | |
| | | | ACCAUCACCAU | | |
| SEQ ID NO: | | 37 | 38 | 3 | 4 |

SEQ ID NO: 178 consists of from 5′ end to 3′ end, 5′ UTR SEQ
ID NO: 3, mRNA ORF SEQ ID NO: 38, and 3′ UTR SEQ ID NO: 4.

| | St_FliC_nI | METPAQLLFLLLLWL | AUGGAAACCCCUGC | GGGAAA | UGAUAA |
|---|---|---|---|---|---|
| | gK | PDTTGAQVINTNSLS | UCAGCUGCUGUUCC | UAAGAG | UAGGCU |
| | | LLTQNNLNKSQSALG | UGCUGCUGCUGUGG | AGAAAA | GGAGCC |
| | | TAIERLSSGLRINSAK | CUGCCUGAUACAAC | GAAGAG | UCGGUG |
| | | DDAAGQAIANRFTA | AGGCGCCCAAGUGA | UAAGAA | GCCAUG |
| | | NIKGLTQASRNANDG | UCAACACCAACAGC | GAAAUA | CUUCUU |
| | | ISIAQTTEGALNEINN | CUGAGCCUGCUGAC | UAAGAG | GCCCCU |
| | | NLQRVRELAVQSAN | CCAGAACAACCUGA | CCACC | UGGGCC |
| | | GTNSQSDLDSIQAEIT | ACAAGAGCCAGAGC | | UCCCCC |
| | | QRLNEIDRVSGQTQF | GCCCUGGGCACAGC | | CAGCCC |
| | | NGVKVLAQDNTLTIQ | CAUCGAAAGACUGU | | CUCCUC |
| | | VGANDGETIDIDLKEI | CUAGCGGCCUGCGG | | CCCUUC |
| | | SSKTLGLDKLNVQDA | AUCAACAGCGCCAA | | CUGCAC |
| | | YTPKETAVTVDKTTY | AGAUGAUGCUGCCG | | CCGUAC |
| | | KNGTDPITAQSNTDI | GACAGGCCAUUGCC | | CCCCGU |
| | | QTAIGGGATGVTGA | AACAGAUUCACCGC | | GGUCUU |
| | | DIKFKDGQYYLDVK | CAACAUCAAGGGCC | | UGAAUA |
| | | GGASAGVYKATYDE | UGACACAGGCCAGC | | AAGUCU |
| | | TTKKVNIDTTDKTPL | AGAAACGCCAACGA | | GAGUGG |
| | | ATAEATAIRGTATITH | CGGCAUCUCUAUCG | | GCGGC |
| | | NQIAEVTKEGVDTTT | CCCAGACAACAGAA | | |
| | | VAAQLAAAGVTGAD | GGCGCCCUGAACGA | | |
| | | KDNTSLVKLSFEDKN | GAUCAACAACAACC | | |
| | | GKVIDGGYAVKMGD | UGCAGAGAGUGCGC | | |
| | | DFYAATYDEKTGAIT | GAGCUGGCCGUGCA | | |
| | | AKTTTYTDGTGVAQ | AUCUGCCAAUGGCA | | |
| | | TGAVKFGGANGKSE | CAAACAGCCAGUCC | | |
| | | VVTATDGKTYLASD | GACCUGGAUUCCAU | | |
| | | LDKHNFRTGGELKEV | CCAGGCCGAGAUCA | | |
| | | NTDKTENPLQKIDAA | CCCAGCGGCUGAAU | | |
| | | LAQVDTLRSDLGAV | GAGAUCGACAGAGU | | |
| | | QNRFNSAITNLGNTV | GUCCGGCCAGACAC | | |
| | | NNLSSARSRIEDSDY | AGUUCAACGGCGUG | | |
| | | ATEVSNMSRAQILQQ | AAAGUGCUGGCCCA | | |
| | | AGTSVLAQANQVPQ | GGACAACACCCUGA | | |
| | | NVLSLLR | CCAUCCAAGUGGGA | | |
| | | | GCCAACGAUGGCGA | | |
| | | | GACAAUCGACAUCG | | |
| | | | ACCUGAAAGAGAUC | | |
| | | | UCCAGCAAGACCCU | | |
| | | | GGGCCUCGACAAGC | | |
| | | | UGAACGUGCAGGAU | | |
| | | | GCCUACACACCCAA | | |
| | | | AGAAACCGCCGUGA | | |
| | | | CCGUGGACAAGACC | | |
| | | | ACCUACAAGAACGG | | |
| | | | CACAGACCCCAUCA | | |
| | | | CAGCCCAGAGCAAC | | |
| | | | ACCGAUAUCCAGAC | | |
| | | | CGCCAUUGGAGGCG | | |
| | | | GAGCUACAGGUGUU | | |
| | | | ACAGGCGCCGACAU | | |
| | | | CAAGUUCAAGGACG | | |
| | | | GCCAGUACUACCUG | | |
| | | | GACGUGAAAGGCGG | | |
| | | | AGCAUCUGCCGGCG | | |
| | | | UGUACAAGGCCACA | | |
| | | | UACGACGAAACCAC | | |
| | | | CAAGAAAGUGAACA | | |
| | | | UCGACACCACCGAC | | |
| | | | AAGACCCCUCUGGC | | |
| | | | CACAGCUGAAGCCA | | |
| | | | CAGCCAUUAGAGGC | | |
| | | | ACCGCCACAAUCAC | | |
| | | | CCACAACCAGAUCG | | |
| | | | CCGAAGUGACCAAA | | |
| | | | GAAGGCGUGGACAC | | |
| | | | CACAACCGUGGCUG | | |
| | | | CUCAACUUGCUGCU | | |
| | | | GCUGGCGUUACCGG | | |
| | | | CGCUGACAAGGAUA | | |
| | | | AUACCAGCCUGGUC | | |
| | | | AAGCUGAGCUUCGA | | |
| | | | GGACAAGAAUGGCA | | |
| | | | AAGUGAUCGACGGC | | |
| | | | GGCUACGCCGUGAA | | |
| | | | GAUGGGCGACGAUU | | |
| | | | UCUACGCCGCCACC | | |
| | | | UACGAUGAGAAGAC | | |
| | | | CGGCGCCAUUACCG | | |
| | | | CCAAGACCACAACC | | |
| | | | UACACAGAUGGCAC | | |
| | | | AGGCGUGGCACAGA | | |
| | | | CAGGGGCCGUGAAA | | |
| | | | UUUGGAGGCGCCAA | | |
| | | | CGGCAAGAGCGAGG | | |
| | | | UCGUGACAGCUACC | | |
| | | | GACGGCAAGACAUA | | |
| | | | CCUGGCCAGCGAUC | | |
| | | | UGGACAAGCACAAC | | |
| | | | UUCAGAACAGGCGG | | |
| | | | CGAGCUGAAAGAAG | | |
| | | | UGAACACAGACAAG | | |
| | | | ACCGAGAAUCCGCU | | |
| | | | GCAGAAGAUCGACG | | |
| | | | CUGCUCUGGCACAA | | |
| | | | GUGGACACCCUGAG | | |
| | | | AAGUGAUCUGGGCG | | |
| | | | CCGUGCAGAACAGG | | |
| | | | UUCAACUCCGCCAU | | |
| | | | CACCAACCUGGGCA | | |
| | | | ACACCGUGAACAAU | | |
| | | | CUGAGCAGCGCCAG | | |
| | | | AAGCCGGAUCGAGG | | |
| | | | ACAGCGAUUAUGCC | | |
| | | | ACCGAGGUGUCCAA | | |
| | | | CAUGAGCAGAGCCC | | |
| | | | AGAUUCUGCAGCAG | | |
| | | | GCCGGCACAUCUGU | | |
| | | | UCUGGCUCAGGCAA | | |
| | | | AUCAGGUGCCCCAG | | |
| | | | AACGUGCUGUCCCU | | |
| | | | GCUGAGA | | |
| SEQ ID NO: | | 39 | 40 | 3 | 4 |

SEQ ID NO: 179 consists of from 5′ end to 3′ end, 5′ UTR SEQ
ID NO: 3, mRNA ORF SEQ ID NO: 40, and 3′ UTR SEQ ID NO: 4.

| | SpA_FliC_ | MAQVINTNSLSLLTQ | AUGGCCCAAGUGAU | GGGAAA | UGAUAA |
|---|---|---|---|---|---|
| | cHis | NNLNKSQSALGTAIE | CAACACCAACAGCC | UAAGAG | UAGGCU |
| | | RLSSGLRINSAKDDA | UGAGCCUGCUGACC | AGAAAA | GGAGCC |
| | | AGQAIANRFTANIKG | CAGAACAACCUGAA | GAAGAG | UCGGUG |
| | | LTQASRNANDGISIA | CAAGAGCCAGAGCG | UAAGAA | GCCAUG |
| | | QTTEGALNEINNNLQ | CCCUGGGCACAGCC | GAAAUA | CUUCUU |
| | | RVRELAVQSANSTNS | AUCGAAAGACUGUC | UAAGAG | GCCCCU |
| | | QSDLDSIQAEITQRLN | UAGCGGCCUGCGGA | CCACC | UGGGCC |
| | | EIDRVSGQTQFNGVK | UCAACAGCGCCAAA | | UCCCCC |
| | | VLAQDNTLTIQVGAN | GAUGAUGCUGCCGG | | CAGCCC |
| | | NGETIDIDLKQINSQT | ACAGGCCAUUGCCA | | CUCCUC |
| | | LGLDTLNVQKKYDV | ACAGAUUCACCGCC | | CCCUUC |
| | | KSEAVTPSATLSTTA | AACAUCAAGGGCCU | | CUGCAC |
| | | LDGAGLKTGTGSTTD | GACACAGGCCAGCA | | CCGUAC |
| | | TGSIKDGKVYYNSTS | GAAACGCCAACGAC | | CCCCGU |
| | | KNYYVEVEFTDATD | GGCAUCUCUAUCGC | | GGUCUU |
| | | QTNKGGFYKVNVAD | CCAGACAACAGAAG | | UGAAUA |
| | | DGAVTMTAATTKEA | GCGCCCUGAACGAG | | AAGUCU |
| | | TTPTGITEVTQVQKP | AUCAACAACAACCU | | GAGUGG |
| | | VAAPAAIQAQLTAAH | GCAGAGAGUGCGCG | | GCGGC |
| | | VTGADTAEMVKMSY | AGCUGGCCGUGCAG | | |
| | | TDKNGKTIDGGFGVK | UCUGCCAAUAGCAC | | |
| | | VGADIYAATKNKDG | AAACAGCCAGUCCG | | |
| | | SFSINTTEYTDKDGN | ACCUGGACAGCAUC | | |
| | | TKTALNQLGGADGK | CAGGCCGAGAUCAC | | |
| | | TEVVSIDGKTYNASK | CCAGCGGCUGAAUG | | |
| | | AAGHNFKAQPELAE | AGAUCGACAGAGUG | | |
| | | AAAATTENPLAKIDA | UCCGGCCAGACACA | | |
| | | ALAQVDALRSDLGA | GUUCAACGGCGUGA | | |
| | | VQNRFNSAITNLGNT | AAGUGCUGGCCCAG | | |
| | | VNNLSSARSRIEDSD | GACAACACCCUGAC | | |
| | | YATEVSNMSRAQILQ | CAUCCAAGUGGGAG | | |
| | | QAGTSVLAQANQVP | CCAACAACGGCGAG | | |
| | | QNVLSLLRHHHHHH | ACAAUCGACAUCGA | | |
| | | | CCUGAAGCAGAUCA | | |
| | | | ACUCUCAGACCCUG | | |
| | | | GGCCUCGACACCCU | | |
| | | | GAACGUGCAGAAGA | | |
| | | | AGUACGACGUCAAG | | |
| | | | AGCGAGGCCGUGAC | | |
| | | | ACCUAGCGCCACAC | | |
| | | | UGUCUACAACAGCC | | |
| | | | CUGGAUGGCGCCGG | | |
| | | | ACUCAAGACAGGCA | | |
| | | | CAGGCAGCACAACA | | |
| | | | GACACCGGCUCCAU | | |
| | | | CAAGGACGGCAAGG | | |
| | | | UGUACUACAACUCC | | |
| | | | ACCAGCAAGAACUA | | |
| | | | CUACGUCGAGGUGG | | |
| | | | AAUUCACCGACGCC | | |
| | | | ACCGACCAGACAAA | | |
| | | | CAAAGGCGGCUUCU | | |
| | | | ACAAAGUGAACGUG | | |
| | | | GCCGACGAUGGGGC | | |
| | | | CGUGACUAUGACAG | | |
| | | | CCGCCACUACCAAA | | |
| | | | GAGGCCACCACACC | | |
| | | | UACAGGCAUCACCG | | |
| | | | AAGUGACCCAGGUG | | |
| | | | CAGAAACCUGUGGC | | |
| | | | UGCCCCUGCUGCUA | | |
| | | | UUCAGGCCCAACUG | | |
| | | | ACAGCUGCCCAUGU | | |
| | | | GACAGGCGCCGAUA | | |
| | | | CAGCCGAGAUGGUC | | |
| | | | AAGAUGAGCUACAC | | |
| | | | CGACAAGAACGGCA | | |
| | | | AGACCAUUGACGGC | | |
| | | | GGCUUCGGAGUGAA | | |
| | | | AGUGGGCGCCGACA | | |
| | | | UCUAUGCCGCCACC | | |
| | | | AAGAACAAGGAUGG | | |
| | | | CAGCUUCAGCAUCA | | |
| | | | AUACCACCGAGUAC | | |
| | | | ACCGAUAAGGACGG | | |
| | | | GAACACCAAGACAG | | |
| | | | CCCUGAACCAGCUU | | |
| | | | GGCGGAGCCGAUGG | | |
| | | | AAAGACCGAGGUGG | | |
| | | | UGUCUAUCGAUGGC | | |
| | | | AAGACCUACAACGC | | |
| | | | CAGCAAGGCCGCUG | | |
| | | | GCCACAACUUUAAG | | |
| | | | GCUCAGCCUGAACU | | |
| | | | GGCCGAAGCUGCCG | | |
| | | | CUGCUACCACAGAG | | |
| | | | AAUCCUCUGGCCAA | | |
| | | | GAUCGAUGCCGCUC | | |
| | | | UGGCACAAGUGGAU | | |
| | | | GCCCUGAGAAGUGA | | |
| | | | UCUGGGCGCCGUGC | | |
| | | | AGAACAGGUUCAAC | | |
| | | | UCCGCCAUCACCAA | | |
| | | | CCUGGGCAACACCG | | |
| | | | UGAACAAUCUGAGC | | |
| | | | AGCGCCAGAAGCCG | | |
| | | | GAUCGAGGACAGCG | | |
| | | | AUUAUGCCACCGAG | | |
| | | | GUGUCCAACAUGAG | | |
| | | | CAGAGCCCAGAUUC | | |
| | | | UGCAGCAGGCCGGC | | |
| | | | ACAUCUGUUCUGGC | | |
| | | | UCAGGCAAAUCAGG | | |
| | | | UGCCCCAGAACGUG | | |
| | | | CUGUCCCUGCUGAG | | |
| | | | ACACCAUCACCACC | | |
| | | | ACCAU | | |
| SEQ ID NO: | | 41 | 42 | 3 | 4 |

SEQ ID NO: 180 consists of from 5′ end to 3′ end, 5′ UTR SEQ
ID NO: 3, mRNA ORF SEQ ID NO: 42, and 3′ UTR SEQ ID NO: 4.

| | SpA_FliC | MAQVINTNSLSLLTQ | AUGGCCCAAGUGAU | GGGAAA | UGAUAA |
|---|---|---|---|---|---|
| | | NNLNKSQSALGTAIE | CAACACCAACAGCC | UAAGAG | UAGGCU |
| | | RLSSGLRINSAKDDA | UGAGCCUGCUGACC | AGAAAA | GGAGCC |
| | | AGQAIANRFTANIKG | CAGAACAACCUGAA | GAAGAG | UCGGUG |
| | | LTQASRNANDGISIA | CAAGAGCCAGAGCG | UAAGAA | GCCAUG |
| | | QTTEGALNEINNNLQ | CCCUGGGCACAGCC | GAAAUA | CUUCUU |
| | | RVRELAVQSANSTNS | AUCGAAAGACUGUC | UAAGAG | GCCCCU |
| | | QSDLDSIQAEITQRLN | UAGCGGCCUGCGGA | CCACC | UGGGCC |
| | | EIDRVSGQTQFNGVK | UCAACAGCGCCAAA | | UCCCCC |
| | | VLAQDNTLTIQVGAN | GAUGAUGCUGCCGG | | CAGCCC |
| | | NGETIDIDLKQINSQT | ACAGGCCAUUGCCA | | CUCCUC |
| | | LGLDTLNVQKKYDV | ACAGAUUCACCGCC | | CCCUUC |
| | | KSEAVTPSATLSTTA | AACAUCAAGGGCCU | | CUGCAC |
| | | LDGAGLKTGTGSTTD | GACACAGGCCAGCA | | CCGUAC |
| | | TGSIKDGKVYYNSTS | GAAACGCCAACGAC | | CCCCGU |
| | | KNYYVEVEFTDATD | GGCAUCUCUAUCGC | | GGUCUU |
| | | QTNKGGFYKVNVAD | CCAGACAACAGAAG | | UGAAUA |
| | | DGAVTMTAATTKEA | GCGCCCUGAACGAG | | AAGUCU |
| | | TTPTGITEVTQVQKP | AUCAACAACAACCU | | GAGUGG |
| | | VAAPAAIQAQLTAAH | GCAGAGAGUGCGCG | | GCGGC |
| | | VTGADTAEMVKMSY | AGCUGGCCGUGCAG | | |
| | | TDKNGKTIDGGFGVK | UCUGCCAAUAGCAC | | |
| | | VGADIYAATKNKDG | AAACAGCCAGUCCG | | |
| | | SFSINTTEYTDKDGN | ACCUGGACAGCAUC | | |
| | | TKTALNQLGGADGK | CAGGCCGAGAUCAC | | |
| | | TEVVSIDGKTYNASK | CCAGCGGCUGAAUG | | |
| | | AAGHNFKAQPELAE | AGAUCGACAGAGUG | | |
| | | AAAATTENPLAKIDA | UCCGGCCAGACACA | | |
| | | ALAQVDALRSDLGA | GUUCAACGGCGUGA | | |
| | | VQNRFNSAITNLGNT | AAGUGCUGGCCCAG | | |
| | | VNNLSSARSRIEDSD | GACAACACCCUGAC | | |
| | | YATEVSNMSRAQILQ | CAUCCAAGUGGGAG | | |
| | | QAGTSVLAQANQVP | CCAACAACGGCGAG | | |
| | | QNVLSLLR | ACAAUCGACAUCGA | | |
| | | | CCUGAAGCAGAUCA | | |
| | | | ACUCUCAGACCCUG | | |
| | | | GGCCUCGACACCCU | | |
| | | | GAACGUGCAGAAGA | | |
| | | | AGUACGACGUCAAG | | |
| | | | AGCGAGGCCGUGAC | | |
| | | | ACCUAGCGCCACAC | | |
| | | | UGUCUACAACAGCC | | |
| | | | CUGGAUGGCGCCGG | | |
| | | | ACUCAAGACAGGCA | | |
| | | | CAGGCAGCACAACA | | |
| | | | GACACCGGCUCCAU | | |
| | | | CAAGGACGGCAAGG | | |
| | | | UGUACUACAACUCC | | |
| | | | ACCAGCAAGAACUA | | |
| | | | CUACGUCGAGGUGG | | |
| | | | AAUUCACCGACGCC | | |
| | | | ACCGACCAGACAAA | | |
| | | | CAAAGGCGGCUUCU | | |
| | | | ACAAAGUGAACGUG | | |
| | | | GCCGACGAUGGGGC | | |
| | | | CGUGACUAUGACAG | | |
| | | | CCGCCACUACCAAA | | |
| | | | GAGGCCACCACACC | | |
| | | | UACAGGCAUCACCG | | |
| | | | AAGUGACCCAGGUG | | |
| | | | CAGAAACCUGUGGC | | |
| | | | UGCCCCUGCUGCUA | | |
| | | | UUCAGGCCCAACUG | | |
| | | | ACAGCUGCCCAUGU | | |
| | | | GACAGGCGCCGAUA | | |
| | | | CAGCCGAGAUGGUC | | |
| | | | AAGAUGAGCUACAC | | |
| | | | CGACAAGAACGGCA | | |
| | | | AGACCAUUGACGGC | | |
| | | | GGCUUCGGAGUGAA | | |
| | | | AGUGGGCGCCGACA | | |
| | | | UCUAUGCCGCCACC | | |
| | | | AAGAACAAGGAUGG | | |
| | | | CAGCUUCAGCAUCA | | |
| | | | AUACCACCGAGUAC | | |
| | | | ACCGAUAAGGACGG | | |
| | | | GAACACCAAGACAG | | |
| | | | CCCUGAACCAGCUU | | |
| | | | GGCGGAGCCGAUGG | | |
| | | | AAAGACCGAGGUGG | | |
| | | | UGUCUAUCGAUGGC | | |
| | | | AAGACCUACAACGC | | |
| | | | CAGCAAGGCCGCUG | | |
| | | | GCCACAACUUUAAG | | |
| | | | GCUCAGCCUGAACU | | |
| | | | GGCCGAAGCUGCCG | | |
| | | | CUGCUACCACAGAG | | |
| | | | AAUCCUCUGGCCAA | | |
| | | | GAUCGAUGCCGCUC | | |
| | | | UGGCACAAGUGGAU | | |
| | | | GCCCUGAGAAGUGA | | |
| | | | UCUGGGCGCCGUGC | | |
| | | | AGAACAGGUUCAAC | | |
| | | | UCCGCCAUCACCAA | | |
| | | | CCUGGGCAACACCG | | |
| | | | UGAACAAUCUGAGC | | |
| | | | AGCGCCAGAAGCCG | | |
| | | | GAUCGAGGACAGCG | | |
| | | | AUUAUGCCACCGAG | | |
| | | | GUGUCCAACAUGAG | | |
| | | | CAGAGCCCAGAUUC | | |
| | | | UGCAGCAGGCCGGC | | |
| | | | ACAUCUGUUCUGGC | | |
| | | | UCAGGCAAAUCAGG | | |
| | | | UGCCCCAGAACGUG | | |
| | | | CUGUCCCUGCUGAG | | |
| | | | A | | |
| SEQ ID NO: | | 43 | 44 | 3 | 4 |

SEQ ID NO: 181 consists of from 5′ end to 3′ end, 5′ UTR SEQ
ID NO: 3, mRNA ORF SEQ ID NO: 44, and 3′ UTR SEQ ID NO: 4.

| | SpA_FliC_ | METPAQLLFLLLLWL | AUGGAAACCCCUGC | GGGAAA | UGAUAA |
|---|---|---|---|---|---|
| | nIgK_cHis | PDTTAQVINTNSLSLL | UCAGCUGCUGUUCC | UAAGAG | UAGGCU |
| | | TQNNLNKSQSALGTA | UGCUGCUGCUGUGG | AGAAAA | GGAGCC |
| | | IERLSSGLRINSAKDD | CUGCCUGAUACAAC | GAAGAG | UCGGUG |
| | | AAGQAIANRFTANIK | AGCCCAAGUGAUCA | UAAGAA | GCCAUG |
| | | GLTQASRNANDGISI | ACACCAACAGCCUG | GAAAUA | CUUCUU |
| | | AQTTEGALNEINNNL | AGCCUGCUGACCCA | UAAGAG | GCCCCU |
| | | QRVRELAVQSANSTN | GAACAACCUGAACA | CCACC | UGGGCC |
| | | SQSDLDSIQAEITQRL | AGAGCCAGAGCGCC | | UCCCCC |
| | | NEIDRVSGQTQFNGV | CUGGGCACAGCCAU | | CAGCCC |
| | | KVLAQDNTLTIQVGA | CGAAAGACUGUCUA | | CUCCUC |
| | | NNGETIDIDLKQINSQ | GCGGCCUGCGGAUC | | CCCUUC |
| | | TLGLDTLNVQKKYD | AACAGCGCCAAAGA | | CUGCAC |
| | | VKSEAVTPSATLSTT | UGAUGCUGCCGGAC | | CCGUAC |
| | | ALDGAGLKTGTGSTT | AGGCCAUUGCCAAC | | CCCCGU |
| | | DTGSIKDGKVYYNST | AGAUUCACCGCCAA | | GGUCUU |
| | | SKNYYVEVEFTDATD | CAUCAAGGGCCUGA | | UGAAUA |
| | | QTNKGGFYKVNVAD | CACAGGCCAGCAGA | | AAGUCU |
| | | DGAVTMTAATTKEA | AACGCCAACGACGG | | GAGUGG |
| | | TTPTGITEVTQVQKP | CAUCUCUAUCGCCC | | GCGGC |
| | | VAAPAAIQAQLTAAH | AGACAACAGAAGGC | | |
| | | VTGADTAEMVKMSY | GCCCUGAACGAGAU | | |
| | | TDKNGKTIDGGFGVK | CAACAACAACCUGC | | |
| | | VGADIYAATKNKDG | AGAGAGUGCGCGAG | | |
| | | SFSINTTEYTDKDGN | CUGGCCGUGCAGUC | | |
| | | TKTALNQLGGADGK | UGCCAAUAGCACAA | | |
| | | TEVVSIDGKTYNASK | ACAGCCAGUCCGAC | | |
| | | AAGHNFKAQPELAE | CUGGACAGCAUCCA | | |
| | | AAAATTENPLAKIDA | GGCCGAGAUCACCC | | |
| | | ALAQVDALRSDLGA | AGCGGCUGAAUGAG | | |
| | | VQNRFNSAITNLGNT | AUCGACAGAGUGUC | | |
| | | VNNLSSARSRIEDSD | CGGCCAGACACAGU | | |
| | | YATEVSNMSRAQILQ | UCAACGGCGUGAAA | | |
| | | QAGTSVLAQANQVP | GUGCUGGCCCAGGA | | |
| | | QNVLSLLRHHHHHH | CAACACCCUGACCA | | |
| | | | UCCAAGUGGGAGCC | | |
| | | | AACAACGGCGAGAC | | |
| | | | AAUCGACAUCGACC | | |
| | | | UGAAGCAGAUCAAC | | |
| | | | UCUCAGACCCUGGG | | |
| | | | CCUCGACACCCUGA | | |
| | | | ACGUGCAGAAGAAG | | |
| | | | UACGACGUCAAGAG | | |
| | | | CGAGGCCGUGACAC | | |
| | | | CUAGCGCCACACUG | | |
| | | | UCUACAACAGCCCU | | |
| | | | GGAUGGCGCCGGAC | | |
| | | | UCAAGACAGGCACA | | |
| | | | GGCAGCACAACAGA | | |
| | | | CACCGGCUCCAUCA | | |
| | | | AGGACGGCAAGGUG | | |
| | | | UACUACAACUCCAC | | |
| | | | CAGCAAGAACUACU | | |
| | | | ACGUCGAGGUGGAA | | |
| | | | UUCACCGACGCCAC | | |
| | | | CGACCAGACAAACA | | |
| | | | AAGGCGGCUUCUAC | | |
| | | | AAAGUGAACGUGGC | | |
| | | | CGACGAUGGGGCCG | | |
| | | | UGACUAUGACAGCC | | |
| | | | GCCACUACCAAAGA | | |
| | | | GGCCACCACACCUA | | |
| | | | CAGGCAUCACCGAA | | |
| | | | GUGACCCAGGUGCA | | |
| | | | GAAACCUGUGGCUG | | |
| | | | CCCCUGCUGCUAUU | | |
| | | | CAGGCCCAACUGAC | | |
| | | | AGCUGCCCAUGUGA | | |
| | | | CAGGCGCCGAUACA | | |
| | | | GCCGAGAUGGUCAA | | |
| | | | GAUGAGCUACACCG | | |
| | | | ACAAGAACGGCAAG | | |
| | | | ACCAUUGACGGCGG | | |
| | | | CUUCGGAGUGAAAG | | |
| | | | UGGGCGCCGACAUC | | |
| | | | UAUGCCGCCACCAA | | |
| | | | GAACAAGGAUGGCA | | |
| | | | GCUUCAGCAUCAAU | | |
| | | | ACCACCGAGUACAC | | |
| | | | CGAUAAGGACGGGA | | |
| | | | ACACCAAGACAGCC | | |
| | | | CUGAACCAGCUUGG | | |
| | | | CGGAGCCGAUGGAA | | |
| | | | AGACCGAGGUGGUG | | |
| | | | UCUAUCGAUGGCAA | | |
| | | | GACCUACAACGCCA | | |
| | | | GCAAGGCCGCUGGC | | |
| | | | CACAACUUUAAGGC | | |
| | | | UCAGCCUGAACUGG | | |
| | | | CCGAAGCUGCCGCU | | |
| | | | GCUACCACAGAGAA | | |
| | | | UCCUCUGGCCAAGA | | |
| | | | UCGAUGCCGCUCUG | | |
| | | | GCACAAGUGGAUGC | | |
| | | | CCUGAGAAGUGAUC | | |
| | | | UGGGCGCCGUGCAG | | |
| | | | AACAGGUUCAACUC | | |
| | | | CGCCAUCACCAACC | | |
| | | | UGGGCAACACCGUG | | |
| | | | AACAAUCUGAGCAG | | |
| | | | CGCCAGAAGCCGGA | | |
| | | | UCGAGGACAGCGAU | | |
| | | | UAUGCCACCGAGGU | | |
| | | | GUCCAACAUGAGCA | | |
| | | | GAGCCCAGAUUCUG | | |
| | | | CAGCAGGCCGGCAC | | |
| | | | AUCUGUUCUGGCUC | | |
| | | | AGGCAAAUCAGGUG | | |
| | | | CCCCAGAACGUGCU | | |
| | | | GUCCCUGCUGAGAC | | |
| | | | ACCAUCACCACCAC | | |
| | | | CAU | | |
| SEQ ID NO: | | 45 | 46 | 3 | 4 |

SEQ ID NO: 182 consists of from 5′ end to 3′ end, 5′ UTR SEQ
ID NO: 3, mRNA ORF SEQ ID NO: 46, and 3′ UTR SEQ ID NO: 4.

| | SpA_FliC_ | METPAQLLFLLLLWL | AUGGAAACCCCUGC | GGGAAA | UGAUAA |
|---|---|---|---|---|---|
| | nIgK | PDTTAQVINTNSLSLL | UCAGCUGCUGUUCC | UAAGAG | UAGGCU |
| | | TQNNLNKSQSALGTA | UGCUGCUGCUGUGG | AGAAAA | GGAGCC |
| | | IERLSSGLRINSAKDD | CUGCCUGAUACAAC | GAAGAG | UCGGUG |
| | | AAGQAIANRFTANIK | AGCCCAAGUGAUCA | UAAGAA | GCCAUG |
| | | GLTQASRNANDGISI | ACACCAACAGCCUG | GAAAUA | CUUCUU |
| | | AQTTEGALNEINNNL | AGCCUGCUGACCCA | UAAGAG | GCCCCU |
| | | QRVRELAVQSANSTN | GAACAACCUGAACA | CCACC | UGGGCC |
| | | SQSDLDSIQAEITQRL | AGAGCCAGAGCGCC | | UCCCCC |
| | | NEIDRVSGQTQFNGV | CUGGGCACAGCCAU | | CAGCCC |
| | | KVLAQDNTLTIQVGA | CGAAAGACUGUCUA | | CUCCUC |
| | | NNGETIDIDLKQINSQ | GCGGCCUGCGGAUC | | CCCUUC |
| | | TLGLDTLNVQKKYD | AACAGCGCCAAAGA | | CUGCAC |
| | | VKSEAVTPSATLSTT | UGAUGCUGCCGGAC | | CCGUAC |
| | | ALDGAGLKTGTGSTT | AGGCCAUUGCCAAC | | CCCCGU |
| | | DTGSIKDGKVYYNST | AGAUUCACCGCCAA | | GGUCUU |
| | | SKNYYVEVEFTDATD | CAUCAAGGGCCUGA | | UGAAUA |
| | | QTNKGGFYKVNVAD | CACAGGCCAGCAGA | | AAGUCU |
| | | DGAVTMTAATTKEA | AACGCCAACGACGG | | GAGUGG |
| | | TTPTGITEVTQVQKP | CAUCUCUAUCGCCC | | GCGGC |
| | | VAAPAAIQAQLTAAH | AGACAACAGAAGGC | | |
| | | VTGADTAEMVKMSY | GCCCUGAACGAGAU | | |
| | | TDKNGKTIDGGFGVK | CAACAACAACCUGC | | |
| | | VGADIYAATKNKDG | AGAGAGUGCGCGAG | | |
| | | SFSINTTEYTDKDGN | CUGGCCGUGCAGUC | | |
| | | TKTALNQLGGADGK | UGCCAAUAGCACAA | | |
| | | TEVVSIDGKTYNASK | ACAGCCAGUCCGAC | | |
| | | AAGHNFKAQPELAE | CUGGACAGCAUCCA | | |
| | | AAAATTENPLAKIDA | GGCCGAGAUCACCC | | |
| | | ALAQVDALRSDLGA | AGCGGCUGAAUGAG | | |
| | | VQNRFNSAITNLGNT | AUCGACAGAGUGUC | | |
| | | VNNLSSARSRIEDSD | CGGCCAGACACAGU | | |
| | | YATEVSNMSRAQILQ | UCAACGGCGUGAAA | | |
| | | QAGTSVLAQANQVP | GUGCUGGCCCAGGA | | |
| | | QNVLSLLR | CAACACCCUGACCA | | |
| | | | UCCAAGUGGGAGCC | | |
| | | | AACAACGGCGAGAC | | |
| | | | AAUCGACAUCGACC | | |
| | | | UGAAGCAGAUCAAC | | |
| | | | UCUCAGACCCUGGG | | |
| | | | CCUCGACACCCUGA | | |
| | | | ACGUGCAGAAGAAG | | |
| | | | UACGACGUCAAGAG | | |
| | | | CGAGGCCGUGACAC | | |
| | | | CUAGCGCCACACUG | | |
| | | | UCUACAACAGCCCU | | |
| | | | GGAUGGCGCCGGAC | | |
| | | | UCAAGACAGGCACA | | |
| | | | GGCAGCACAACAGA | | |
| | | | CACCGGCUCCAUCA | | |
| | | | AGGACGGCAAGGUG | | |
| | | | UACUACAACUCCAC | | |
| | | | CAGCAAGAACUACU | | |
| | | | ACGUCGAGGUGGAA | | |
| | | | UUCACCGACGCCAC | | |
| | | | CGACCAGACAAACA | | |
| | | | AAGGCGGCUUCUAC | | |
| | | | AAAGUGAACGUGGC | | |
| | | | CGACGAUGGGGCCG | | |
| | | | UGACUAUGACAGCC | | |
| | | | GCCACUACCAAAGA | | |
| | | | GGCCACCACACCUA | | |
| | | | CAGGCAUCACCGAA | | |
| | | | GUGACCCAGGUGCA | | |
| | | | GAAACCUGUGGCUG | | |
| | | | CCCCUGCUGCUAUU | | |
| | | | CAGGCCCAACUGAC | | |
| | | | AGCUGCCCAUGUGA | | |
| | | | CAGGCGCCGAUACA | | |
| | | | GCCGAGAUGGUCAA | | |
| | | | GAUGAGCUACACCG | | |
| | | | ACAAGAACGGCAAG | | |
| | | | ACCAUUGACGGCGG | | |
| | | | CUUCGGAGUGAAAG | | |
| | | | UGGGCGCCGACAUC | | |
| | | | UAUGCCGCCACCAA | | |
| | | | GAACAAGGAUGGCA | | |
| | | | GCUUCAGCAUCAAU | | |
| | | | ACCACCGAGUACAC | | |
| | | | CGAUAAGGACGGGA | | |
| | | | ACACCAAGACAGCC | | |
| | | | CUGAACCAGCUUGG | | |
| | | | CGGAGCCGAUGGAA | | |
| | | | AGACCGAGGUGGUG | | |
| | | | UCUAUCGAUGGCAA | | |
| | | | GACCUACAACGCCA | | |
| | | | GCAAGGCCGCUGGC | | |
| | | | CACAACUUUAAGGC | | |
| | | | UCAGCCUGAACUGG | | |
| | | | CCGAAGCUGCCGCU | | |
| | | | GCUACCACAGAGAA | | |
| | | | UCCUCUGGCCAAGA | | |
| | | | UCGAUGCCGCUCUG | | |
| | | | GCACAAGUGGAUGC | | |
| | | | CCUGAGAAGUGAUC | | |
| | | | UGGGCGCCGUGCAG | | |
| | | | AACAGGUUCAACUC | | |
| | | | CGCCAUCACCAACC | | |
| | | | UGGGCAACACCGUG | | |
| | | | AACAAUCUGAGCAG | | |
| | | | CGCCAGAAGCCGGA | | |
| | | | UCGAGGACAGCGAU | | |
| | | | UAUGCCACCGAGGU | | |
| | | | GUCCAACAUGAGCA | | |
| | | | GAGCCCAGAUUCUG | | |
| | | | CAGCAGGCCGGCAC | | |
| | | | AUCUGUUCUGGCUC | | |
| | | | AGGCAAAUCAGGUG | | |
| | | | CCCCAGAACGUGCU | | |
| | | | GUCCCUGCUGAGA | | |
| SEQ ID NO: | | 47 | 48 | 3 | 4 |

SEQ ID NO: 183 consists of from 5′ end to 3′ end, 5′ UTR SEQ
ID NO: 3, mRNA ORF SEQ ID NO: 48, and 3′ UTR SEQ ID NO: 4.

| | Stm_FliC_ | MAQVINTNSLSLLTQ | AUGGCCCAAGUGAU | GGGAAA | UGAUAA |
|---|---|---|---|---|---|
| | cHis | NNLNKSQSALGTAIE | CAACACCAACAGCC | UAAGAG | UAGGCU |
| | | RLSSGLRINSAKDDA | UGAGCCUGCUGACC | AGAAAA | GGAGCC |
| | | AGQAIANRFTANIKG | CAGAACAACCUGAA | GAAGAG | UCGGUG |
| | | LTQASRNANDGISIA | CAAGAGCCAGAGCG | UAAGAA | GCCAUG |
| | | QTTEGALNEINNNLQ | CCCUGGGCACAGCC | GAAAUA | CUUCUU |
| | | RVRELAVQSANSTNS | AUCGAAAGACUGUC | UAAGAG | GCCCCU |
| | | QSDLDSIQAEITQRLN | UAGCGGCCUGCGGA | CCACC | UGGGCC |
| | | EIDRVSGQTQFNGVK | UCAACAGCGCCAAA | | UCCCCC |
| | | VLAQDNTLTIQVGAN | GAUGAUGCUGCCGG | | CAGCCC |
| | | DGETIDIDLKQINSQT | ACAGGCCAUUGCCA | | CUCCUC |
| | | LGLDTLNVQQKYKV | ACAGAUUCACCGCC | | CCCUUC |
| | | SDTAATVTGYADTTI | AACAUCAAGGGCCU | | CUGCAC |
| | | ALDNSTFKASATGLG | GACACAGGCCAGCA | | CCGUAC |
| | | GTDQKIDGDLKFDDT | GAAACGCCAACGAC | | CCCCGU |
| | | TGKYYAKVTVTGGT | GGCAUCUCUAUCGC | | GGUCUU |
| | | GKDGYYEVSVDKTN | CCAGACAACAGAAG | | UGAAUA |
| | | GKVTLAGGATSPLTG | GCGCCCUGAACGAG | | AAGUCU |
| | | GLPATATEDVKNVQ | AUCAACAACAACCU | | GAGUGG |
| | | VANADLTEAKAALT | GCAGAGAGUGCGCG | | GCGGC |
| | | AAGVTGTASVVKMS | AGCUGGCCGUGCAG | | |
| | | YTDNNGKTIDGGLA | UCUGCCAAUAGCAC | | |
| | | VKVGDDYYSATQNK | AAACAGCCAGUCCG | | |
| | | DGSISINTTKYTADD | ACCUGGACAGCAUC | | |
| | | GTSKTALNKLGGAD | CAGGCCGAGAUCAC | | |
| | | GKTEVVSIGGKTYAA | CCAGCGGCUGAAUG | | |
| | | SKAEGHNFKAQPDL | AGAUCGACAGAGUG | | |
| | | AEAAATTTENPLQKI | UCCGGCCAGACACA | | |
| | | DAALAQVDTLRSDL | GUUCAACGGCGUGA | | |
| | | GAVQNRFNSAITNLG | AAGUGCUGGCCCAG | | |
| | | NTVNNLTSARSRIED | GACAACACCCUGAC | | |
| | | SDYATEVSNMSRAQI | CAUCCAAGUGGGAG | | |
| | | LQQAGTSVLAQANQ | CCAACGAUGGCGAG | | |
| | | VPQNVLSLLRHHHH | ACAAUCGACAUCGA | | |
| | | HH | CCUGAAGCAGAUCA | | |
| | | | ACUCUCAGACCCUG | | |
| | | | GGCCUCGACACCCU | | |
| | | | GAACGUGCAGCAGA | | |
| | | | AGUACAAGGUUUCC | | |
| | | | GACACCGCCGCCAC | | |
| | | | CGUGACAGGCUAUG | | |
| | | | CCGAUACAACAAUC | | |
| | | | GCCCUGGACAACAG | | |
| | | | CACCUUCAAGGCCU | | |
| | | | CUGCCACAGGCCUC | | |
| | | | GGAGGCACCGAUCA | | |
| | | | GAAGAUUGACGGCG | | |
| | | | AUCUGAAGUUCGAC | | |
| | | | GACACCACCGGCAA | | |
| | | | GUACUACGCCAAAG | | |
| | | | UGACAGUGACAGGC | | |
| | | | GGCACAGGCAAGGA | | |
| | | | CGGCUACUACGAAG | | |
| | | | UGUCCGUGGACAAG | | |
| | | | ACCAACGGCAAAGU | | |
| | | | GACUCUGGCUGGCG | | |
| | | | GAGCCACCUCUCCU | | |
| | | | CUUACUGGUGGACU | | |
| | | | UCCUGCCACCGCCA | | |
| | | | CCGAGGACGUGAAG | | |
| | | | AAUGUGCAGGUCGC | | |
| | | | CAACGCCGAUCUGA | | |
| | | | CCGAAGCUAAAGCC | | |
| | | | GCUCUGACAGCUGC | | |
| | | | UGGCGUGACCGGAA | | |
| | | | CAGCCUCUGUGGUC | | |
| | | | AAGAUGAGCUACAC | | |
| | | | CGACAACAACGGCA | | |
| | | | AGACCAUCGACGGC | | |
| | | | GGACUGGCAGUGAA | | |
| | | | AGUGGGCGACGAUU | | |
| | | | ACUACAGCGCCACA | | |
| | | | CAGAACAAGGAUGG | | |
| | | | CAGCAUCUCCAUCA | | |
| | | | AUACCACCAAGUAC | | |
| | | | ACCGCCGACGAUGG | | |
| | | | CACCUCUAAGACCG | | |
| | | | CUCUGAACAAACUC | | |
| | | | GGCGGAGCCGAUGG | | |
| | | | CAAGACCGAGGUGG | | |
| | | | UGUCUAUCGGCGGC | | |
| | | | AAGACAUACGCCGC | | |
| | | | CUCUAAGGCCGAGG | | |
| | | | GCCACAACUUUAAA | | |
| | | | GCCCAGCCUGAUCU | | |
| | | | GGCUGAGGCCGCUG | | |
| | | | CCACUACCACAGAG | | |
| | | | AAUCCUCUGCAGAA | | |
| | | | GAUCGACGCCGCUC | | |
| | | | UGGCACAAGUGGAU | | |
| | | | ACCCUGAGAAGCGA | | |
| | | | UCUGGGCGCCGUGC | | |
| | | | AGAACAGGUUCAAU | | |
| | | | AGCGCCAUCACCAA | | |
| | | | CCUGGGCAACACCG | | |
| | | | UGAACAAUCUGACC | | |
| | | | AGCGCCAGAAGCCG | | |
| | | | GAUCGAGGACAGCG | | |
| | | | AUUAUGCCACAGAG | | |
| | | | GUGUCCAACAUGAG | | |
| | | | CAGAGCCCAGAUCC | | |
| | | | UGCAGCAGGCCGGA | | |
| | | | ACAUCUGUUCUGGC | | |
| | | | UCAGGCCAAUCAGG | | |
| | | | UGCCCCAGAAUGUG | | |
| | | | CUGUCCCUGCUGAG | | |
| | | | ACACCAUCACCACC | | |
| | | | ACCAU | | |
| SEQ ID NO: | | 49 | 50 | 3 | 4 |

SEQ ID NO: 184 consists of from 5′ end to 3′ end, 5′ UTR SEQ
ID NO: 3, mRNA ORF SEQ ID NO: 50, and 3′ UTR SEQ ID NO: 4.

| | Stm_FliC | MAQVINTNSLSLLTQ | AUGGCCCAAGUGAU | GGGAAA | UGAUAA |
|---|---|---|---|---|---|
| | | NNLNKSQSALGTAIE | CAACACCAACAGCC | UAAGAG | UAGGCU |
| | | RLSSGLRINSAKDDA | UGAGCCUGCUGACC | AGAAAA | GGAGCC |
| | | AGQAIANRFTANIKG | CAGAACAACCUGAA | GAAGAG | UCGGUG |
| | | LTQASRNANDGISIA | CAAGAGCCAGAGCG | UAAGAA | GCCAUG |
| | | QTTEGALNEINNNLQ | CCCUGGGCACAGCC | GAAAUA | CUUCUU |
| | | RVRELAVQSANSTNS | AUCGAAAGACUGUC | UAAGAG | GCCCCU |
| | | QSDLDSIQAEITQRLN | UAGCGGCCUGCGGA | CCACC | UGGGCC |
| | | EIDRVSGQTQFNGVK | UCAACAGCGCCAAA | | UCCCCC |
| | | VLAQDNTLTIQVGAN | GAUGAUGCUGCCGG | | CAGCCC |
| | | DGETIDIDLKQINSQT | ACAGGCCAUUGCCA | | CUCCUC |
| | | LGLDTLNVQQKYKV | ACAGAUUCACCGCC | | CCCUUC |
| | | SDTAATVTGYADTTI | AACAUCAAGGGCCU | | CUGCAC |
| | | ALDNSTFKASATGLG | GACACAGGCCAGCA | | CCGUAC |
| | | GTDQKIDGDLKFDDT | GAAACGCCAACGAC | | CCCCGU |
| | | TGKYYAKVTVTGGT | GGCAUCUCUAUCGC | | GGUCUU |
| | | GKDGYYEVSVDKTN | CCAGACAACAGAAG | | UGAAUA |
| | | GKVTLAGGATSPLTG | GCGCCCUGAACGAG | | AAGUCU |
| | | GLPATATEDVKNVQ | AUCAACAACAACCU | | GAGUGG |
| | | VANADLTEAKAALT | GCAGAGAGUGCGCG | | GCGGC |
| | | AAGVTGTASVVKMS | AGCUGGCCGUGCAG | | |
| | | YTDNNGKTIDGGLA | UCUGCCAAUAGCAC | | |
| | | VKVGDDYYSATQNK | AAACAGCCAGUCCG | | |
| | | DGSISINTTKYTADD | ACCUGGACAGCAUC | | |
| | | GTSKTALNKLGGAD | CAGGCCGAGAUCAC | | |
| | | GKTEVVSIGGKTYAA | CCAGCGGCUGAAUG | | |
| | | SKAEGHNFKAQPDL | AGAUCGACAGAGUG | | |
| | | AEAAATTTENPLQKI | UCCGGCCAGACACA | | |
| | | DAALAQVDTLRSDL | GUUCAACGGCGUGA | | |
| | | GAVQNRFNSAITNLG | AAGUGCUGGCCCAG | | |
| | | NTVNNLTSARSRIED | GACAACACCCUGAC | | |
| | | SDYATEVSNMSRAQI | CAUCCAAGUGGGAG | | |
| | | LQQAGTSVLAQANQ | CCAACGAUGGCGAG | | |
| | | VPQNVLSLLR | ACAAUCGACAUCGA | | |
| | | | CCUGAAGCAGAUCA | | |
| | | | ACUCUCAGACCCUG | | |
| | | | GGCCUCGACACCCU | | |
| | | | GAACGUGCAGCAGA | | |
| | | | AGUACAAGGUUUCC | | |
| | | | GACACCGCCGCCAC | | |
| | | | CGUGACAGGCUAUG | | |
| | | | CCGAUACAACAAUC | | |
| | | | GCCCUGGACAACAG | | |
| | | | CACCUUCAAGGCCU | | |
| | | | CUGCCACAGGCCUC | | |
| | | | GGAGGCACCGAUCA | | |
| | | | GAAGAUUGACGGCG | | |
| | | | AUCUGAAGUUCGAC | | |
| | | | GACACCACCGGCAA | | |
| | | | GUACUACGCCAAAG | | |
| | | | UGACAGUGACAGGC | | |
| | | | GGCACAGGCAAGGA | | |
| | | | CGGCUACUACGAAG | | |
| | | | UGUCCGUGGACAAG | | |
| | | | ACCAACGGCAAAGU | | |
| | | | GACUCUGGCUGGCG | | |
| | | | GAGCCACCUCUCCU | | |
| | | | CUUACUGGUGGACU | | |
| | | | UCCUGCCACCGCCA | | |
| | | | CCGAGGACGUGAAG | | |
| | | | AAUGUGCAGGUCGC | | |
| | | | CAACGCCGAUCUGA | | |
| | | | CCGAAGCUAAAGCC | | |
| | | | GCUCUGACAGCUGC | | |
| | | | UGGCGUGACCGGAA | | |
| | | | CAGCCUCUGUGGUC | | |
| | | | AAGAUGAGCUACAC | | |
| | | | CGACAACAACGGCA | | |
| | | | AGACCAUCGACGGC | | |
| | | | GGACUGGCAGUGAA | | |
| | | | AGUGGGCGACGAUU | | |
| | | | ACUACAGCGCCACA | | |
| | | | CAGAACAAGGAUGG | | |
| | | | CAGCAUCUCCAUCA | | |
| | | | AUACCACCAAGUAC | | |
| | | | ACCGCCGACGAUGG | | |
| | | | CACCUCUAAGACCG | | |
| | | | CUCUGAACAAACUC | | |
| | | | GGCGGAGCCGAUGG | | |
| | | | CAAGACCGAGGUGG | | |
| | | | UGUCUAUCGGCGGC | | |
| | | | AAGACAUACGCCGC | | |
| | | | CUCUAAGGCCGAGG | | |
| | | | GCCACAACUUUAAA | | |
| | | | GCCCAGCCUGAUCU | | |
| | | | GGCUGAGGCCGCUG | | |
| | | | CCACUACCACAGAG | | |
| | | | AAUCCUCUGCAGAA | | |
| | | | GAUCGACGCCGCUC | | |
| | | | UGGCACAAGUGGAU | | |
| | | | ACCCUGAGAAGCGA | | |
| | | | UCUGGGCGCCGUGC | | |
| | | | AGAACAGGUUCAAU | | |
| | | | AGCGCCAUCACCAA | | |
| | | | CCUGGGCAACACCG | | |
| | | | UGAACAAUCUGACC | | |
| | | | AGCGCCAGAAGCCG | | |
| | | | GAUCGAGGACAGCG | | |
| | | | AUUAUGCCACAGAG | | |
| | | | GUGUCCAACAUGAG | | |
| | | | CAGAGCCCAGAUCC | | |
| | | | UGCAGCAGGCCGGA | | |
| | | | ACAUCUGUUCUGGC | | |
| | | | UCAGGCCAAUCAGG | | |
| | | | UGCCCCAGAAUGUG | | |
| | | | CUGUCCCUGCUGAG | | |
| | | | A | | |
| SEQ ID NO: | | 51 | 52 | 3 | 4 |

SEQ ID NO: 185 consists of from 5′ end to 3′ end, 5′ UTR SEQ
ID NO: 3, mRNA ORF SEQ ID NO: 52, and 3′ UTR SEQ ID NO: 4.

| | Stm_FliC_ | METPAQLLFLLLLWL | AUGGAAACCCCUGC | GGGAAA | UGAUAA |
|---|---|---|---|---|---|
| | nIgK_cHis | PDTTAQVINTNSLSLL | UCAGCUGCUGUUCC | UAAGAG | UAGGCU |
| | | TQNNLNKSQSALGTA | UGCUGCUGCUGUGG | AGAAAA | GGAGCC |
| | | IERLSSGLRINSAKDD | CUGCCUGAUACAAC | GAAGAG | UCGGUG |
| | | AAGQAIANRFTANIK | AGCCCAAGUGAUCA | UAAGAA | GCCAUG |
| | | GLTQASRNANDGISI | ACACCAACAGCCUG | GAAAUA | CUUCUU |
| | | AQTTEGALNEINNNL | AGCCUGCUGACCCA | UAAGAG | GCCCCU |
| | | QRVRELAVQSANSTN | GAACAACCUGAACA | CCACC | UGGGCC |
| | | SQSDLDSIQAEITQRL | AGAGCCAGAGCGCC | | UCCCCC |
| | | NEIDRVSGQTQFNGV | CUGGGCACAGCCAU | | CAGCCC |
| | | KVLAQDNTLTIQVGA | CGAAAGACUGUCUA | | CUCCUC |
| | | NDGETIDIDLKQINSQ | GCGGCCUGCGGAUC | | CCCUUC |
| | | TLGLDTLNVQQKYK | AACAGCGCCAAAGA | | CUGCAC |
| | | VSDTAATVTGYADT | UGAUGCUGCCGGAC | | CCGUAC |
| | | TIALDNSTFKASATG | AGGCCAUUGCCAAC | | CCCCGU |
| | | LGGTDQKIDGDLKFD | AGAUUCACCGCCAA | | GGUCUU |
| | | DTTGKYYAKVTVTG | CAUCAAGGGCCUGA | | UGAAUA |
| | | GTGKDGYYEVSVDK | CACAGGCCAGCAGA | | AAGUCU |
| | | TNGKVTLAGGATSPL | AACGCCAACGACGG | | GAGUGG |
| | | TGGLPATATEDVKN | CAUCUCUAUCGCCC | | GCGGC |
| | | VQVANADLTEAKAA | AGACAACAGAAGGC | | |
| | | LTAAGVTGTASVVK | GCCCUGAACGAGAU | | |
| | | MSYTDNNGKTIDGG | CAACAACAACCUGC | | |
| | | LAVKVGDDYYSATQ | AGAGAGUGCGCGAG | | |
| | | NKDGSISINTTKYTA | CUGGCCGUGCAGUC | | |
| | | DDGTSKTALNKLGG | UGCCAAUAGCACAA | | |
| | | ADGKTEVVSIGGKTY | ACAGCCAGUCCGAC | | |
| | | AASKAEGHNFKAQP | CUGGACAGCAUCCA | | |
| | | DLAEAAATTTENPLQ | GGCCGAGAUCACCC | | |
| | | KIDAALAQVDTLRSD | AGCGGCUGAAUGAG | | |
| | | LGAVQNRFNSAITNL | AUCGACAGAGUGUC | | |
| | | GNTVNNLTSARSRIE | CGGCCAGACACAGU | | |
| | | DSDYATEVSNMSRA | UCAACGGCGUGAAA | | |
| | | QILQQAGTSVLAQAN | GUGCUGGCCCAGGA | | |
| | | QVPQNVLSLLRHHH | CAACACCCUGACCA | | |
| | | HHH | UCCAAGUGGGAGCC | | |
| | | | AACGAUGGCGAGAC | | |
| | | | AAUCGACAUCGACC | | |
| | | | UGAAGCAGAUCAAC | | |
| | | | UCUCAGACCCUGGG | | |
| | | | CCUCGACACCCUGA | | |
| | | | ACGUGCAGCAGAAG | | |
| | | | UACAAGGUUUCCGA | | |
| | | | CACCGCCGCCACCG | | |
| | | | UGACAGGCUAUGCC | | |
| | | | GAUACAACAAUCGC | | |
| | | | CCUGGACAACAGCA | | |
| | | | CCUUCAAGGCCUCU | | |
| | | | GCCACAGGCCUCGG | | |
| | | | AGGCACCGAUCAGA | | |
| | | | AGAUUGACGGCGAU | | |
| | | | CUGAAGUUCGACGA | | |
| | | | CACCACCGGCAAGU | | |
| | | | ACUACGCCAAAGUG | | |
| | | | ACAGUGACAGGCGG | | |
| | | | CACAGGCAAGGACG | | |
| | | | GCUACUACGAAGUG | | |
| | | | UCCGUGGACAAGAC | | |
| | | | CAACGGCAAAGUGA | | |
| | | | CUCUGGCUGGCGGA | | |
| | | | GCCACCUCUCCUCU | | |
| | | | UACUGGUGGACUUC | | |
| | | | CUGCCACCGCCACC | | |
| | | | GAGGACGUGAAGAA | | |
| | | | UGUGCAGGUCGCCA | | |
| | | | ACGCCGAUCUGACC | | |
| | | | GAAGCUAAAGCCGC | | |
| | | | UCUGACAGCUGCUG | | |
| | | | GCGUGACCGGAACA | | |
| | | | GCCUCUGUGGUCAA | | |
| | | | GAUGAGCUACACCG | | |
| | | | ACAACAACGGCAAG | | |
| | | | ACCAUCGACGGCGG | | |
| | | | ACUGGCAGUGAAAG | | |
| | | | UGGGCGACGAUUAC | | |
| | | | UACAGCGCCACACA | | |
| | | | GAACAAGGAUGGCA | | |
| | | | GCAUCUCCAUCAAU | | |
| | | | ACCACCAAGUACAC | | |
| | | | CGCCGACGAUGGCA | | |
| | | | CCUCUAAGACCGCU | | |
| | | | CUGAACAAACUCGG | | |
| | | | CGGAGCCGAUGGCA | | |
| | | | AGACCGAGGUGGUG | | |
| | | | UCUAUCGGCGGCAA | | |
| | | | GACAUACGCCGCCU | | |
| | | | CUAAGGCCGAGGGC | | |
| | | | CACAACUUUAAAGC | | |
| | | | CCAGCCUGAUCUGG | | |
| | | | CUGAGGCCGCUGCC | | |
| | | | ACUACCACAGAGAA | | |
| | | | UCCUCUGCAGAAGA | | |
| | | | UCGACGCCGCUCUG | | |
| | | | GCACAAGUGGAUAC | | |
| | | | CCUGAGAAGCGAUC | | |
| | | | UGGGCGCCGUGCAG | | |
| | | | AACAGGUUCAAUAG | | |
| | | | CGCCAUCACCAACC | | |
| | | | UGGGCAACACCGUG | | |
| | | | AACAAUCUGACCAG | | |
| | | | CGCCAGAAGCCGGA | | |
| | | | UCGAGGACAGCGAU | | |
| | | | UAUGCCACAGAGGU | | |
| | | | GUCCAACAUGAGCA | | |
| | | | GAGCCCAGAUCCUG | | |
| | | | CAGCAGGCCGGAAC | | |
| | | | AUCUGUUCUGGCUC | | |
| | | | AGGCCAAUCAGGUG | | |
| | | | CCCCAGAAUGUGCU | | |
| | | | GUCCCUGCUGAGAC | | |
| | | | ACCAUCACCACCAC | | |
| | | | CAU | | |
| SEQ ID NO: | | 53 | 54 | 3 | 4 |

SEQ ID NO: 186 consists of from 5′ end to 3′ end, 5′ UTR SEQ
ID NO: 3, mRNA ORF SEQ ID NO: 54, and 3′ UTR SEQ ID NO: 4.

| | Stm_FliC_ | METPAQLLFLLLLWL | AUGGAAACCCCUGC | GGGAAA | UGAUAA |
|---|---|---|---|---|---|
| | nIgK | PDTTAQVINTNSLSLL | UCAGCUGCUGUUCC | UAAGAG | UAGGCU |
| | | TQNNLNKSQSALGTA | UGCUGCUGCUGUGG | AGAAAA | GGAGCC |
| | | IERLSSGLRINSAKDD | CUGCCUGAUACAAC | GAAGAG | UCGGUG |
| | | AAGQAIANRFTANIK | AGCCCAAGUGAUCA | UAAGAA | GCCAUG |
| | | GLTQASRNANDGISI | ACACCAACAGCCUG | GAAAUA | CUUCUU |
| | | AQTTEGALNEINNNL | AGCCUGCUGACCCA | UAAGAG | GCCCCU |
| | | QRVRELAVQSANSTN | GAACAACCUGAACA | CCACC | UGGGCC |
| | | SQSDLDSIQAEITQRL | AGAGCCAGAGCGCC | | UCCCCC |
| | | NEIDRVSGQTQFNGV | CUGGGCACAGCCAU | | CAGCCC |
| | | KVLAQDNTLTIQVGA | CGAAAGACUGUCUA | | CUCCUC |
| | | NDGETIDIDLKQINSQ | GCGGCCUGCGGAUC | | CCCUUC |
| | | TLGLDTLNVQQKYK | AACAGCGCCAAAGA | | CUGCAC |
| | | VSDTAATVTGYADT | UGAUGCUGCCGGAC | | CCGUAC |
| | | TIALDNSTFKASATG | AGGCCAUUGCCAAC | | CCCCGU |
| | | LGGTDQKIDGDLKFD | AGAUUCACCGCCAA | | GGUCUU |
| | | DTTGKYYAKVTVTG | CAUCAAGGGCCUGA | | UGAAUA |
| | | GTGKDGYYEVSVDK | CACAGGCCAGCAGA | | AAGUCU |
| | | TNGKVTLAGGATSPL | AACGCCAACGACGG | | GAGUGG |
| | | TGGLPATATEDVKN | CAUCUCUAUCGCCC | | GCGGC |
| | | VQVANADLTEAKAA | AGACAACAGAAGGC | | |
| | | LTAAGVTGTASVVK | GCCCUGAACGAGAU | | |
| | | MSYTDNNGKTIDGG | CAACAACAACCUGC | | |
| | | LAVKVGDDYYSATQ | AGAGAGUGCGCGAG | | |
| | | NKDGSISINTTKYTA | CUGGCCGUGCAGUC | | |
| | | DDGTSKTALNKLGG | UGCCAAUAGCACAA | | |
| | | ADGKTEVVSIGGKTY | ACAGCCAGUCCGAC | | |
| | | AASKAEGHNFKAQP | CUGGACAGCAUCCA | | |
| | | DLAEAAATTTENPLQ | GGCCGAGAUCACCC | | |
| | | KIDAALAQVDTLRSD | AGCGGCUGAAUGAG | | |
| | | LGAVQNRFNSAITNL | AUCGACAGAGUGUC | | |
| | | GNTVNNLTSARSRIE | CGGCCAGACACAGU | | |
| | | DSDYATEVSNMSRA | UCAACGGCGUGAAA | | |
| | | QILQQAGTSVLAQAN | GUGCUGGCCCAGGA | | |
| | | QVPQNVLSLLR | CAACACCCUGACCA | | |
| | | | UCCAAGUGGGAGCC | | |
| | | | AACGAUGGCGAGAC | | |
| | | | AAUCGACAUCGACC | | |
| | | | UGAAGCAGAUCAAC | | |
| | | | UCUCAGACCCUGGG | | |
| | | | CCUCGACACCCUGA | | |
| | | | ACGUGCAGCAGAAG | | |
| | | | UACAAGGUUUCCGA | | |
| | | | CACCGCCGCCACCG | | |
| | | | UGACAGGCUAUGCC | | |
| | | | GAUACAACAAUCGC | | |
| | | | CCUGGACAACAGCA | | |
| | | | CCUUCAAGGCCUCU | | |
| | | | GCCACAGGCCUCGG | | |
| | | | AGGCACCGAUCAGA | | |
| | | | AGAUUGACGGCGAU | | |
| | | | CUGAAGUUCGACGA | | |
| | | | CACCACCGGCAAGU | | |
| | | | ACUACGCCAAAGUG | | |
| | | | ACAGUGACAGGCGG | | |
| | | | CACAGGCAAGGACG | | |
| | | | GCUACUACGAAGUG | | |
| | | | UCCGUGGACAAGAC | | |
| | | | CAACGGCAAAGUGA | | |
| | | | CUCUGGCUGGCGGA | | |
| | | | GCCACCUCUCCUCU | | |
| | | | UACUGGUGGACUUC | | |
| | | | CUGCCACCGCCACC | | |
| | | | GAGGACGUGAAGAA | | |
| | | | UGUGCAGGUCGCCA | | |
| | | | ACGCCGAUCUGACC | | |
| | | | GAAGCUAAAGCCGC | | |
| | | | UCUGACAGCUGCUG | | |
| | | | GCGUGACCGGAACA | | |
| | | | GCCUCUGUGGUCAA | | |
| | | | GAUGAGCUACACCG | | |
| | | | ACAACAACGGCAAG | | |
| | | | ACCAUCGACGGCGG | | |
| | | | ACUGGCAGUGAAAG | | |
| | | | UGGGCGACGAUUAC | | |
| | | | UACAGCGCCACACA | | |
| | | | GAACAAGGAUGGCA | | |
| | | | GCAUCUCCAUCAAU | | |
| | | | ACCACCAAGUACAC | | |
| | | | CGCCGACGAUGGCA | | |
| | | | CCUCUAAGACCGCU | | |
| | | | CUGAACAAACUCGG | | |
| | | | CGGAGCCGAUGGCA | | |
| | | | AGACCGAGGUGGUG | | |
| | | | UCUAUCGGCGGCAA | | |
| | | | GACAUACGCCGCCU | | |
| | | | CUAAGGCCGAGGGC | | |
| | | | CACAACUUUAAAGC | | |
| | | | CCAGCCUGAUCUGG | | |
| | | | CUGAGGCCGCUGCC | | |
| | | | ACUACCACAGAGAA | | |
| | | | UCCUCUGCAGAAGA | | |
| | | | UCGACGCCGCUCUG | | |
| | | | GCACAAGUGGAUAC | | |
| | | | CCUGAGAAGCGAUC | | |
| | | | UGGGCGCCGUGCAG | | |
| | | | AACAGGUUCAAUAG | | |
| | | | CGCCAUCACCAACC | | |
| | | | UGGGCAACACCGUG | | |
| | | | AACAAUCUGACCAG | | |
| | | | CGCCAGAAGCCGGA | | |
| | | | UCGAGGACAGCGAU | | |
| | | | UAUGCCACAGAGGU | | |
| | | | GUCCAACAUGAGCA | | |
| | | | GAGCCCAGAUCCUG | | |
| | | | CAGCAGGCCGGAAC | | |
| | | | AUCUGUUCUGGCUC | | |
| | | | AGGCCAAUCAGGUG | | |
| | | | CCCCAGAAUGUGCU | | |
| | | | GUCCCUGCUGAGA | | |
| SEQ ID NO: | | 55 | 56 | 3 | 4 |

SEQ ID NO: 187 consists of from 5′ end to 3′ end, 5′ UTR SEQ
ID NO: 3, mRNA ORF SEQ ID NO: 56, and 3′ UTR SEQ ID NO: 4.

| | St_Mig14 | MKIQEVKRILTRWQP | AUGAAGAUCCAGGA | GGGAAA | UGAUAA |
|---|---|---|---|---|---|
| | | SSFTLYREVFTQYGG | GGUGAAGAGAAUCC | UAAGAG | UAGGCU |
| | | SINMHPDIVDYFMKR | UCACCCGAUGGCAG | AGAAAA | GGAGCC |
| | | HNWHFKFFHYKEDD | CCUAGCAGCUUCAC | GAAGAG | UCGGUG |
| | | KIKGAYFICNDQNIGI | CCUAUACAGAGAGG | UAAGAA | GCCAUG |
| | | LTRRTFPLSSDEILIPM | UGUUCACCCAGUAC | GAAAUA | CUUCUU |
| | | APDLRCFLPDRTNRL | GGCGGCAGCAUCAA | UAAGAG | GCCCCU |
| | | SALHQPQIRNAIWKL | CAUGCACCCGGACA | CCACC | UGGGCC |
| | | ARKKQNCLVKETFSS | UCGUGGACUACUUC | | UCCCCC |
| | | KFEKTRRNEYQRFLK | AUGAAGAGACACAA | | CAGCCC |
| | | KGGSVKSVADCSSDE | CUGGCACUUCAAGU | | CUCCUC |
| | | LTHIFIELFRSRFGNTS | UCUUCCACUACAAG | | CCCUUC |
| | | SCYPADNLANFFSQL | GAGGACGACAAGAU | | CUGCAC |
| | | HHLLFGHILYIEGIPC | CAAGGGCGCGUAUU | | CCGUAC |
| | | AFDIVLKSESQMNVY | UCAUCUGCAACGAC | | CCCCGU |
| | | FDVSNGAIKNECRPL | CAGAACAUCGGCAU | | GGUCUU |
| | | SPGSILMWLNISRAR | CCUAACACGAAGAA | | UGAAUA |
| | | HYCQERQKKLLFSIGI | CCUUCCCUCUGAGC | | AAGUCU |
| | | LKPEWEYKRMWSTP | AGCGACGAGAUCCU | | GAGUGG |
| | | YFTGKSIC | GAUCCCUAUGGCCC | | GCGGC |
| | | | CUGACCUGAGAUGC | | |
| | | | UUCCUGCCUGACAG | | |
| | | | AACCAACAGACUGA | | |
| | | | GCGCCCUGCACCAG | | |
| | | | CCUCAGAUCAGAAA | | |
| | | | CGCCAUCUGGAAGC | | |
| | | | UGGCCAGAAAGAAG | | |
| | | | CAGAACUGCCUGGU | | |
| | | | GAAGGAAACGUUCA | | |
| | | | GCAGCAAGUUCGAG | | |
| | | | AAGACUCGGCGCAA | | |
| | | | CGAGUACCAGAGAU | | |
| | | | UCCUGAAGAAGGGA | | |
| | | | GGUAGCGUCAAGAG | | |
| | | | CGUGGCCGACUGCA | | |
| | | | GUUCGGACGAACUG | | |
| | | | ACCCACAUCUUCAU | | |
| | | | CGAGCUGUUCCGAU | | |
| | | | CCAGAUUCGGCAAC | | |
| | | | ACCAGCAGCUGCUA | | |
| | | | CCCUGCCGACAACC | | |
| | | | UGGCCAACUUCUUC | | |
| | | | AGCCAGCUGCACCA | | |
| | | | CCUGCUGUUCGGCC | | |
| | | | ACAUCCUGUACAUC | | |
| | | | GAGGGCAUCCCUUG | | |
| | | | CGCCUUCGACAUCG | | |
| | | | UUUUGAAGAGUGA | | |
| | | | GAGCCAGAUGAACG | | |
| | | | UGUACUUCGACGUG | | |
| | | | AGCAACGGCGCCAU | | |
| | | | CAAGAACGAGUGCA | | |
| | | | GACCGUUGUCCCCU | | |
| | | | GGAUCUAUCCUUAU | | |
| | | | GUGGCUGAACAUCA | | |
| | | | GCCGGGCUCGCCAC | | |
| | | | UACUGCCAGGAGAG | | |
| | | | ACAGAAGAAGCUUC | | |
| | | | UCUUCUCAAUCGGA | | |
| | | | AUCUUAAAGCCUGA | | |
| | | | GUGGGAGUACAAGA | | |
| | | | GAAUGUGGAGCACC | | |
| | | | CCUUACUUCACCGG | | |
| | | | CAAGAGCAUCUGC | | |
| SEQ ID NO: | | 57 | 58 | 3 | 4 |

SEQ ID NO: 188 consists of from 5′ end to 3′ end, 5′ UTR SEQ
ID NO: 3, mRNA ORF SEQ ID NO: 58, and 3′ UTR SEQ ID NO: 4.

| | St_Mig14_ | MKIQEVKRILTRWQP | AUGAAGAUCCAGGA | GGGAAA | UGAUAA |
|---|---|---|---|---|---|
| | cHis | SSFTLYREVFTQYGG | GGUGAAGAGAAUCC | UAAGAG | UAGGCU |
| | | SINMHPDIVDYFMKR | UCACCCGAUGGCAG | AGAAAA | GGAGCC |
| | | HNWHFKFFHYKEDD | CCUAGCAGCUUCAC | GAAGAG | UCGGUG |
| | | KIKGAYFICNDQNIGI | CCUAUACAGAGAGG | UAAGAA | GCCAUG |
| | | LTRRTFPLSSDEILIPM | UGUUCACCCAGUAC | GAAAUA | CUUCUU |
| | | APDLRCFLPDRTNRL | GGCGGCAGCAUCAA | UAAGAG | GCCCCU |
| | | SALHQPQIRNAIWKL | CAUGCACCCGGACA | CCACC | UGGGCC |
| | | ARKKQNCLVKETFSS | UCGUGGACUACUUC | | UCCCCC |
| | | KFEKTRRNEYQRFLK | AUGAAGAGACACAA | | CAGCCC |
| | | KGGSVKSVADCSSDE | CUGGCACUUCAAGU | | CUCCUC |
| | | LTHIFIELFRSRFGNTS | UCUUCCACUACAAG | | CCCUUC |
| | | SCYPADNLANFFSQL | GAGGACGACAAGAU | | CUGCAC |
| | | HHLLFGHILYIEGIPC | CAAGGGCGCGUAUU | | CCGUAC |
| | | AFDIVLKSESQMNVY | UCAUCUGCAACGAC | | CCCCGU |
| | | FDVSNGAIKNECRPL | CAGAACAUCGGCAU | | GGUCUU |
| | | SPGSILMWLNISRAR | CCUAACACGAAGAA | | UGAAUA |
| | | HYCQERQKKLLFSIGI | CCUUCCCUCUGAGC | | AAGUCU |
| | | LKPEWEYKRMWSTP | AGCGACGAGAUCCU | | GAGUGG |
| | | YFTGKSICHHHHHH | GAUCCCUAUGGCCC | | GCGGC |
| | | | CUGACCUGAGAUGC | | |
| | | | UUCCUGCCUGACAG | | |
| | | | AACCAACAGACUGA | | |
| | | | GCGCCCUGCACCAG | | |
| | | | CCUCAGAUCAGAAA | | |
| | | | CGCCAUCUGGAAGC | | |
| | | | UGGCCAGAAAGAAG | | |
| | | | CAGAACUGCCUGGU | | |
| | | | GAAGGAAACGUUCA | | |
| | | | GCAGCAAGUUCGAG | | |
| | | | AAGACUCGGCGCAA | | |
| | | | CGAGUACCAGAGAU | | |
| | | | UCCUGAAGAAGGGA | | |
| | | | GGUAGCGUCAAGAG | | |
| | | | CGUGGCCGACUGCA | | |
| | | | GUUCGGACGAACUG | | |
| | | | ACCCACAUCUUCAU | | |
| | | | CGAGCUGUUCCGAU | | |
| | | | CCAGAUUCGGCAAC | | |
| | | | ACCAGCAGCUGCUA | | |
| | | | CCCUGCCGACAACC | | |
| | | | UGGCCAACUUCUUC | | |
| | | | AGCCAGCUGCACCA | | |
| | | | CCUGCUGUUCGGCC | | |
| | | | ACAUCCUGUACAUC | | |
| | | | GAGGGCAUCCCUUG | | |
| | | | CGCCUUCGACAUCG | | |
| | | | UUUUGAAGAGUGA | | |
| | | | GAGCCAGAUGAACG | | |
| | | | UGUACUUCGACGUG | | |
| | | | AGCAACGGCGCCAU | | |
| | | | CAAGAACGAGUGCA | | |
| | | | GACCGUUGUCCCCU | | |
| | | | GGAUCUAUCCUUAU | | |
| | | | GUGGCUGAACAUCA | | |
| | | | GCCGGGCUCGCCAC | | |
| | | | UACUGCCAGGAGAG | | |
| | | | ACAGAAGAAGCUUC | | |
| | | | UCUUCUCAAUCGGA | | |
| | | | AUCUUAAAGCCUGA | | |
| | | | GUGGGAGUACAAGA | | |
| | | | GAAUGUGGAGCACC | | |
| | | | CCUUACUUCACCGG | | |
| | | | CAAGAGCAUCUGCC | | |
| | | | ACCACCAUCAUCAC | | |
| | | | CAC | | |
| SEQ ID NO: | | 59 | 60 | 3 | 4 |

SEQ ID NO: 189 consists of from 5′ end to 3′ end, 5′ UTR SEQ
ID NO: 3, mRNA ORF SEQ ID NO: 60, and 3′ UTR SEQ ID NO: 4.

| | St_Mig14_ | METPAQLLLLLLLWL | AUGGAGACUCCAGC | GGGAAA | UGAUAA |
|---|---|---|---|---|---|
| | nIgK | PDTTGKIQEVKRILTR | CCAGCUCCUCCUCC | UAAGAG | UAGGCU |
| | | WQPSSFTLYREVFTQ | UCCUCUUGCUCUGG | AGAAAA | GGAGCC |
| | | YGGSINMHPDIVDYF | UUGCCGGACACCAC | GAAGAG | UCGGUG |
| | | MKRHNWHFKFFHYK | CGGCAAGAUCCAGG | UAAGAA | GCCAUG |
| | | EDDKIKGAYFICNDQ | AGGUGAAGAGAAUC | GAAAUA | CUUCUU |
| | | NIGILTRRTFPLSSDEI | CUCACCCGAUGGCA | UAAGAG | GCCCCU |
| | | LIPMAPDLRCFLPDRT | GCCUAGCAGCUUCA | CCACC | UGGGCC |
| | | NRLSALHQPQIRNAI | CCCUAUACAGAGAG | | UCCCCC |
| | | WKLARKKQNCLVKE | GUGUUCACCCAGUA | | CAGCCC |
| | | TFSSKFEKTRRNEYQ | CGGCGGCAGCAUCA | | CUCCUC |
| | | RFLKKGGSVKSVAD | ACAUGCACCCGGAC | | CCCUUC |
| | | CSSDELTHIFIELFRSR | AUCGUGGACUACUU | | CUGCAC |
| | | FGNTSSCYPADNLAN | CAUGAAGAGACACA | | CCGUAC |
| | | FFSQLHHLLFGHILYI | ACUGGCACUUCAAG | | CCCCGU |
| | | EGIPCAFDIVLKSESQ | UUCUUCCACUACAA | | GGUCUU |
| | | MNVYFDVSNGAIKN | GGAGGACGACAAGA | | UGAAUA |
| | | ECRPLSPGSILMWLNI | UCAAGGGCGCGUAU | | AAGUCU |
| | | SRARHYCQERQKKL | UUCAUCUGCAACGA | | GAGUGG |
| | | LFSIGILKPEWEYKR | CCAGAACAUCGGCA | | GCGGC |
| | | MWSTPYFTGKSIC | UCCUAACACGAAGA | | |
| | | | ACCUUCCCUCUGAG | | |
| | | | CAGCGACGAGAUCC | | |
| | | | UGAUCCCUAUGGCC | | |
| | | | CCUGACCUGAGAUG | | |
| | | | CUUCCUGCCUGACA | | |
| | | | GAACCAACAGACUG | | |
| | | | AGCGCCCUGCACCA | | |
| | | | GCCUCAGAUCAGAA | | |
| | | | ACGCCAUCUGGAAG | | |
| | | | CUGGCCAGAAAGAA | | |
| | | | GCAGAACUGCCUGG | | |
| | | | UGAAGGAAACGUUC | | |
| | | | AGCAGCAAGUUCGA | | |
| | | | GAAGACUCGGCGCA | | |
| | | | ACGAGUACCAGAGA | | |
| | | | UUCCUGAAGAAGGG | | |
| | | | AGGUAGCGUCAAGA | | |
| | | | GCGUGGCCGACUGC | | |
| | | | AGUUCGGACGAACU | | |
| | | | GACCCACAUCUUCA | | |
| | | | UCGAGCUGUUCCGA | | |
| | | | UCCAGAUUCGGCAA | | |
| | | | CACCAGCAGCUGCU | | |
| | | | ACCCUGCCGACAAC | | |
| | | | CUGGCCAACUUCUU | | |
| | | | CAGCCAGCUGCACC | | |
| | | | ACCUGCUGUUCGGC | | |
| | | | CACAUCCUGUACAU | | |
| | | | CGAGGGCAUCCCUU | | |
| | | | GCGCCUUCGACAUC | | |
| | | | GUUUUGAAGAGUG | | |
| | | | AGAGCCAGAUGAAC | | |
| | | | GUGUACUUCGACGU | | |
| | | | GAGCAACGGCGCCA | | |
| | | | UCAAGAACGAGUGC | | |
| | | | AGACCGUUGUCCCC | | |
| | | | UGGAUCUAUCCUUA | | |
| | | | UGUGGCUGAACAUC | | |
| | | | AGCCGGGCUCGCCA | | |
| | | | CUACUGCCAGGAGA | | |
| | | | GACAGAAGAAGCUU | | |
| | | | CUCUUCUCAAUCGG | | |
| | | | AAUCUUAAAGCCUG | | |
| | | | AGUGGGAGUACAAG | | |
| | | | AGAAUGUGGAGCAC | | |
| | | | CCCUUACUUCACCG | | |
| | | | GCAAGAGCAUCUGC | | |
| SEQ ID NO: | | 61 | 62 | 3 | 4 |

SEQ ID NO: 190 consists of from 5′ end to 3′ end, 5′ UTR SEQ
ID NO: 3, mRNA ORF SEQ ID NO: 62, and 3′ UTR SEQ ID NO: 4.

| | St_Mig14_ | METPAQLLLLLLLWL | AUGGAGACUCCAGC | GGGAAA | UGAUAA |
|---|---|---|---|---|---|
| | nIgK_cHis | PDTTGKIQEVKRILTR | CCAGCUCCUCCUCC | UAAGAG | UAGGCU |
| | | WQPSSFTLYREVFTQ | UCCUCUUGCUCUGG | AGAAAA | GGAGCC |
| | | YGGSINMHPDIVDYF | UUGCCGGACACCAC | GAAGAG | UCGGUG |
| | | MKRHNWHFKFFHYK | CGGCAAGAUCCAGG | UAAGAA | GCCAUG |
| | | EDDKIKGAYFICNDQ | AGGUGAAGAGAAUC | GAAAUA | CUUCUU |
| | | NIGILTRRTFPLSSDEI | CUCACCCGAUGGCA | UAAGAG | GCCCCU |
| | | LIPMAPDLRCFLPDRT | GCCUAGCAGCUUCA | CCACC | UGGGCC |
| | | NRLSALHQPQIRNAI | CCCUAUACAGAGAG | | UCCCCC |
| | | WKLARKKQNCLVKE | GUGUUCACCCAGUA | | CAGCCC |
| | | TFSSKFEKTRRNEYQ | CGGCGGCAGCAUCA | | CUCCUC |
| | | RFLKKGGSVKSVAD | ACAUGCACCCGGAC | | CCCUUC |
| | | CSSDELTHIFIELFRSR | AUCGUGGACUACUU | | CUGCAC |
| | | FGNTSSCYPADNLAN | CAUGAAGAGACACA | | CCGUAC |
| | | FFSQLHHLLFGHILYI | ACUGGCACUUCAAG | | CCCCGU |
| | | EGIPCAFDIVLKSESQ | UUCUUCCACUACAA | | GGUCUU |
| | | MNVYFDVSNGAIKN | GGAGGACGACAAGA | | UGAAUA |
| | | ECRPLSPGSILMWLNI | UCAAGGGCGCGUAU | | AAGUCU |
| | | SRARHYCQERQKKL | UUCAUCUGCAACGA | | GAGUGG |
| | | LFSIGILKPEWEYKR | CCAGAACAUCGGCA | | GCGGC |
| | | MWSTPYFTGKSICHH | UCCUAACACGAAGA | | |
| | | HHHH | ACCUUCCCUCUGAG | | |
| | | | CAGCGACGAGAUCC | | |
| | | | UGAUCCCUAUGGCC | | |
| | | | CCUGACCUGAGAUG | | |
| | | | CUUCCUGCCUGACA | | |
| | | | GAACCAACAGACUG | | |
| | | | AGCGCCCUGCACCA | | |
| | | | GCCUCAGAUCAGAA | | |
| | | | ACGCCAUCUGGAAG | | |
| | | | CUGGCCAGAAAGAA | | |
| | | | GCAGAACUGCCUGG | | |
| | | | UGAAGGAAACGUUC | | |
| | | | AGCAGCAAGUUCGA | | |
| | | | GAAGACUCGGCGCA | | |
| | | | ACGAGUACCAGAGA | | |
| | | | UUCCUGAAGAAGGG | | |
| | | | AGGUAGCGUCAAGA | | |
| | | | GCGUGGCCGACUGC | | |
| | | | AGUUCGGACGAACU | | |
| | | | GACCCACAUCUUCA | | |
| | | | UCGAGCUGUUCCGA | | |
| | | | UCCAGAUUCGGCAA | | |
| | | | CACCAGCAGCUGCU | | |
| | | | ACCCUGCCGACAAC | | |
| | | | CUGGCCAACUUCUU | | |
| | | | CAGCCAGCUGCACC | | |
| | | | ACCUGCUGUUCGGC | | |
| | | | CACAUCCUGUACAU | | |
| | | | CGAGGGCAUCCCUU | | |
| | | | GCGCCUUCGACAUC | | |
| | | | GUUUUGAAGAGUG | | |
| | | | AGAGCCAGAUGAAC | | |
| | | | GUGUACUUCGACGU | | |
| | | | GAGCAACGGCGCCA | | |
| | | | UCAAGAACGAGUGC | | |
| | | | AGACCGUUGUCCCC | | |
| | | | UGGAUCUAUCCUUA | | |
| | | | UGUGGCUGAACAUC | | |
| | | | AGCCGGGCUCGCCA | | |
| | | | CUACUGCCAGGAGA | | |
| | | | GACAGAAGAAGCUU | | |
| | | | CUCUUCUCAAUCGG | | |
| | | | AAUCUUAAAGCCUG | | |
| | | | AGUGGGAGUACAAG | | |
| | | | AGAAUGUGGAGCAC | | |
| | | | CCCUUACUUCACCG | | |
| | | | GCAAGAGCAUCUGC | | |
| | | | CACCACCAUCAUCA | | |
| | | | CCAC | | |
| SEQ ID NO: | | 63 | 64 | 3 | 4 |

SEQ ID NO: 191 consists of from 5′ end to 3′ end, 5′ UTR SEQ
ID NO: 3, mRNA ORF SEQ ID NO: 64, and 3′ UTR SEQ ID NO: 4.

| | St_Mig14_ | MKIQEVKRILTRWQP | AUGAAGAUCCAGGA | GGGAAA | UGAUAA |
|---|---|---|---|---|---|
| | NGM | SSFTLYREVFTQYGG | GGUGAAGAGAAUCC | UAAGAG | UAGGCU |
| | | SINMHPDIVDYFMKR | UCACCCGAUGGCAG | AGAAAA | GGAGCC |
| | | HNWHFKFFHYKEDD | CCUAGCAGCUUCAC | GAAGAG | UCGGUG |
| | | KIKGAYFICNDQNIGI | CCUAUACAGAGAGG | UAAGAA | GCCAUG |
| | | LTRRTFPLSSDEILIPM | UGUUCACCCAGUAC | GAAAUA | CUUCUU |
| | | APDLRCFLPDRTNRL | GGCGGCAGCAUCAA | UAAGAG | GCCCCU |
| | | SALHQPQIRNAIWKL | CAUGCACCCGGACA | CCACC | UGGGCC |
| | | ARKKQNCLVKETFSS | UCGUGGACUACUUC | | UCCCCC |
| | | KFEKTRRNEYQRFLK | AUGAAGAGACACAA | | CAGCCC |
| | | KGGSVKSVADCSSDE | CUGGCACUUCAAGU | | CUCCUC |
| | | LTHIFIELFRSRFGNT | UCUUCCACUACAAG | | CCCUUC |
| | | ASCYPADNLANFFSQ | GAGGACGACAAGAU | | CUGCAC |
| | | LHHLLFGHILYIEGIP | CAAGGGCGCGUAUU | | CCGUAC |
| | | CAFDIVLKSESQMNV | UCAUCUGCAACGAC | | CCCCGU |
| | | YFDVSNGAIKNECRP | CAGAACAUCGGCAU | | GGUCUU |
| | | LSPGSILMWLNIARA | CCUAACACGAAGAA | | UGAAUA |
| | | RHYCQERQKKLLFSI | CCUUCCCUCUGAGC | | AAGUCU |
| | | GILKPEWEYKRMWS | AGCGACGAGAUCCU | | GAGUGG |
| | | TPYFTGKSIC | GAUCCCUAUGGCCC | | GCGGC |
| | | | CUGACCUGAGAUGC | | |
| | | | UUCCUGCCUGACAG | | |
| | | | AACCAACAGACUGA | | |
| | | | GCGCCCUGCACCAG | | |
| | | | CCUCAGAUCAGAAA | | |
| | | | CGCCAUCUGGAAGC | | |
| | | | UGGCCAGAAAGAAG | | |
| | | | CAGAACUGCCUGGU | | |
| | | | GAAGGAAACGUUCA | | |
| | | | GCAGCAAGUUCGAG | | |
| | | | AAGACUCGGCGCAA | | |
| | | | CGAGUACCAGAGAU | | |
| | | | UCCUGAAGAAGGGA | | |
| | | | GGUAGCGUCAAGAG | | |
| | | | CGUGGCCGACUGCA | | |
| | | | GUUCGGACGAACUG | | |
| | | | ACCCACAUCUUCAU | | |
| | | | CGAGCUGUUCCGAU | | |
| | | | CCAGAUUCGGCAAC | | |
| | | | ACCGCCAGCUGCUA | | |
| | | | CCCUGCCGACAACC | | |
| | | | UGGCCAACUUCUUC | | |
| | | | AGCCAGCUGCACCA | | |
| | | | CCUGCUGUUCGGCC | | |
| | | | ACAUCCUGUACAUC | | |
| | | | GAGGGCAUCCCUUG | | |
| | | | CGCCUUCGACAUCG | | |
| | | | UUUUGAAGAGUGA | | |
| | | | GAGCCAGAUGAACG | | |
| | | | UGUACUUCGACGUG | | |
| | | | AGCAACGGCGCCAU | | |
| | | | CAAGAACGAGUGCA | | |
| | | | GACCGUUGUCCCCU | | |
| | | | GGAUCUAUCCUUAU | | |
| | | | GUGGCUGAACAUCG | | |
| | | | CCCGGGCUCGCCAC | | |
| | | | UACUGCCAGGAGAG | | |
| | | | ACAGAAGAAGCUUC | | |
| | | | UCUUCUCAAUCGGA | | |
| | | | AUCUUAAAGCCUGA | | |
| | | | GUGGGAGUACAAGA | | |
| | | | GAAUGUGGAGCACC | | |
| | | | CCUUACUUCACCGG | | |
| | | | CAAGAGCAUCUGC | | |
| SEQ ID NO: | | 65 | 66 | 3 | 4 |

SEQ ID NO: 192 consists of from 5′ end to 3′ end, 5′ UTR SEQ
ID NO: 3, mRNA ORF SEQ ID NO: 66, and 3′ UTR SEQ ID NO: 4.

| | St_Mig14_ | MKIQEVKRILTRWQP | AUGAAGAUCCAGGA | GGGAAA | UGAUAA |
|---|---|---|---|---|---|
| | NGM_cHi | SSFTLYREVFTQYGG | GGUGAAGAGAAUCC | UAAGAG | UAGGCU |
| | s | SINMHPDIVDYFMKR | UCACCCGAUGGCAG | AGAAAA | GGAGCC |
| | | HNWHFKFFHYKEDD | CCUAGCAGCUUCAC | GAAGAG | UCGGUG |
| | | KIKGAYFICNDQNIGI | CCUAUACAGAGAGG | UAAGAA | GCCAUG |
| | | LTRRTFPLSSDEILIPM | UGUUCACCCAGUAC | GAAAUA | CUUCUU |
| | | APDLRCFLPDRTNRL | GGCGGCAGCAUCAA | UAAGAG | GCCCCU |
| | | SALHQPQIRNAIWKL | CAUGCACCCGGACA | CCACC | UGGGCC |
| | | ARKKQNCLVKETFSS | UCGUGGACUACUUC | | UCCCCC |
| | | KFEKTRRNEYQRFLK | AUGAAGAGACACAA | | CAGCCC |
| | | KGGSVKSVADCSSDE | CUGGCACUUCAAGU | | CUCCUC |
| | | LTHIFIELFRSRFGNT | UCUUCCACUACAAG | | CCCUUC |
| | | ASCYPADNLANFFSQ | GAGGACGACAAGAU | | CUGCAC |
| | | LHHLLFGHILYIEGIP | CAAGGGCGCGUAUU | | CCGUAC |
| | | CAFDIVLKSESQMNV | UCAUCUGCAACGAC | | CCCCGU |
| | | YFDVSNGAIKNECRP | CAGAACAUCGGCAU | | GGUCUU |
| | | LSPGSILMWLNIARA | CCUAACACGAAGAA | | UGAAUA |
| | | RHYCQERQKKLLFSI | CCUUCCCUCUGAGC | | AAGUCU |
| | | GILKPEWEYKRMWS | AGCGACGAGAUCCU | | GAGUGG |
| | | TPYFTGKSICHHHHH | GAUCCCUAUGGCCC | | GCGGC |
| | | H | CUGACCUGAGAUGC | | |
| | | | UUCCUGCCUGACAG | | |
| | | | AACCAACAGACUGA | | |
| | | | GCGCCCUGCACCAG | | |
| | | | CCUCAGAUCAGAAA | | |
| | | | CGCCAUCUGGAAGC | | |
| | | | UGGCCAGAAAGAAG | | |
| | | | CAGAACUGCCUGGU | | |
| | | | GAAGGAAACGUUCA | | |
| | | | GCAGCAAGUUCGAG | | |
| | | | AAGACUCGGCGCAA | | |
| | | | CGAGUACCAGAGAU | | |
| | | | UCCUGAAGAAGGGA | | |
| | | | GGUAGCGUCAAGAG | | |
| | | | CGUGGCCGACUGCA | | |
| | | | GUUCGGACGAACUG | | |
| | | | ACCCACAUCUUCAU | | |
| | | | CGAGCUGUUCCGAU | | |
| | | | CCAGAUUCGGCAAC | | |
| | | | ACCGCCAGCUGCUA | | |
| | | | CCCUGCCGACAACC | | |
| | | | UGGCCAACUUCUUC | | |
| | | | AGCCAGCUGCACCA | | |
| | | | CCUGCUGUUCGGCC | | |
| | | | ACAUCCUGUACAUC | | |
| | | | GAGGGCAUCCCUUG | | |
| | | | CGCCUUCGACAUCG | | |
| | | | UUUUGAAGAGUGA | | |
| | | | GAGCCAGAUGAACG | | |
| | | | UGUACUUCGACGUG | | |
| | | | AGCAACGGCGCCAU | | |
| | | | CAAGAACGAGUGCA | | |
| | | | GACCGUUGUCCCCU | | |
| | | | GGAUCUAUCCUUAU | | |
| | | | GUGGCUGAACAUCG | | |
| | | | CCCGGGCUCGCCAC | | |
| | | | UACUGCCAGGAGAG | | |
| | | | ACAGAAGAAGCUUC | | |
| | | | UCUUCUCAAUCGGA | | |
| | | | AUCUUAAAGCCUGA | | |
| | | | GUGGGAGUACAAGA | | |
| | | | GAAUGUGGAGCACC | | |
| | | | CCUUACUUCACCGG | | |
| | | | CAAGAGCAUCUGCC | | |
| | | | ACCACCAUCAUCAC | | |
| | | | CAC | | |
| SEQ ID NO: | | 67 | 68 | 3 | 4 |

SEQ ID NO: 193 consists of from 5′ end to 3′ end, 5′ UTR SEQ
ID NO: 3, mRNA ORF SEQ ID NO: 68, and 3′ UTR SEQ ID NO: 4.

| | St_Mig14_ | METPAQLLLLLLLWL | AUGGAGACUCCAGC | GGGAAA | UGAUAA |
|---|---|---|---|---|---|
| | NGM_nIg | PDTTGKIQEVKRILTR | CCAGCUCCUCCUCC | UAAGAG | UAGGCU |
| | K | WQPSSFTLYREVFTQ | UCCUCUUGCUCUGG | AGAAAA | GGAGCC |
| | | YGGSINMHPDIVDYF | UUGCCGGACACCAC | GAAGAG | UCGGUG |
| | | MKRHNWHFKFFHYK | CGGCAAGAUCCAGG | UAAGAA | GCCAUG |
| | | EDDKIKGAYFICNDQ | AGGUGAAGAGAAUC | GAAAUA | CUUCUU |
| | | NIGILTRRTFPLSSDEI | CUCACCCGAUGGCA | UAAGAG | GCCCCU |
| | | LIPMAPDLRCFLPDRT | GCCUAGCAGCUUCA | CCACC | UGGGCC |
| | | NRLSALHQPQIRNAI | CCCUAUACAGAGAG | | UCCCCC |
| | | WKLARKKQNCLVKE | GUGUUCACCCAGUA | | CAGCCC |
| | | TFSSKFEKTRRNEYQ | CGGCGGCAGCAUCA | | CUCCUC |
| | | RFLKKGGSVKSVAD | ACAUGCACCCGGAC | | CCCUUC |
| | | CSSDELTHIFIELFRSR | AUCGUGGACUACUU | | CUGCAC |
| | | FGNTASCYPADNLAN | CAUGAAGAGACACA | | CCGUAC |
| | | FFSQLHHLLFGHILYI | ACUGGCACUUCAAG | | CCCCGU |
| | | EGIPCAFDIVLKSESQ | UUCUUCCACUACAA | | GGUCUU |
| | | MNVYFDVSNGAIKN | GGAGGACGACAAGA | | UGAAUA |
| | | ECRPLSPGSILMWLNI | UCAAGGGCGCGUAU | | AAGUCU |
| | | ARARHYCQERQKKL | UUCAUCUGCAACGA | | GAGUGG |
| | | LFSIGILKPEWEYKR | CCAGAACAUCGGCA | | GCGGC |
| | | MWSTPYFTGKSIC | UCCUAACACGAAGA | | |
| | | | ACCUUCCCUCUGAG | | |
| | | | CAGCGACGAGAUCC | | |
| | | | UGAUCCCUAUGGCC | | |
| | | | CCUGACCUGAGAUG | | |
| | | | CUUCCUGCCUGACA | | |
| | | | GAACCAACAGACUG | | |
| | | | AGCGCCCUGCACCA | | |
| | | | GCCUCAGAUCAGAA | | |
| | | | ACGCCAUCUGGAAG | | |
| | | | CUGGCCAGAAAGAA | | |
| | | | GCAGAACUGCCUGG | | |
| | | | UGAAGGAAACGUUC | | |
| | | | AGCAGCAAGUUCGA | | |
| | | | GAAGACUCGGCGCA | | |
| | | | ACGAGUACCAGAGA | | |
| | | | UUCCUGAAGAAGGG | | |
| | | | AGGUAGCGUCAAGA | | |
| | | | GCGUGGCCGACUGC | | |
| | | | AGUUCGGACGAACU | | |
| | | | GACCCACAUCUUCA | | |
| | | | UCGAGCUGUUCCGA | | |
| | | | UCCAGAUUCGGCAA | | |
| | | | CACCGCCAGCUGCU | | |
| | | | ACCCUGCCGACAAC | | |
| | | | CUGGCCAACUUCUU | | |
| | | | CAGCCAGCUGCACC | | |
| | | | ACCUGCUGUUCGGC | | |
| | | | CACAUCCUGUACAU | | |
| | | | CGAGGGCAUCCCUU | | |
| | | | GCGCCUUCGACAUC | | |
| | | | GUUUUGAAGAGUG | | |
| | | | AGAGCCAGAUGAAC | | |
| | | | GUGUACUUCGACGU | | |
| | | | GAGCAACGGCGCCA | | |
| | | | UCAAGAACGAGUGC | | |
| | | | AGACCGUUGUCCCC | | |
| | | | UGGAUCUAUCCUUA | | |
| | | | UGUGGCUGAACAUC | | |
| | | | GCCCGGGCUCGCCA | | |
| | | | CUACUGCCAGGAGA | | |
| | | | GACAGAAGAAGCUU | | |
| | | | CUCUUCUCAAUCGG | | |
| | | | AAUCUUAAAGCCUG | | |
| | | | AGUGGGAGUACAAG | | |
| | | | AGAAUGUGGAGCAC | | |
| | | | CCCUUACUUCACCG | | |
| | | | GCAAGAGCAUCUGC | | |
| SEQ ID NO: | | 69 | 70 | 3 | 4 |

SEQ ID NO: 194 consists of from 5′ end to 3′ end, 5′ UTR SEQ
ID NO: 3, mRNA ORF SEQ ID NO: 70, and 3′ UTR SEQ ID NO: 4.

| | St_Mig14_ | METPAQLLLLLLLWL | AUGGAGACUCCAGC | GGGAAA | UGAUAA |
|---|---|---|---|---|---|
| | NGM_nIg | PDTTGKIQEVKRILTR | CCAGCUCCUCCUCC | UAAGAG | UAGGCU |
| | K_cHis | WQPSSFTLYREVFTQ | UCCUCUUGCUCUGG | AGAAAA | GGAGCC |
| | | YGGSINMHPDIVDYF | UUGCCGGACACCAC | GAAGAG | UCGGUG |
| | | MKRHNWHFKFFHYK | CGGCAAGAUCCAGG | UAAGAA | GCCAUG |
| | | EDDKIKGAYFICNDQ | AGGUGAAGAGAAUC | GAAAUA | CUUCUU |
| | | NIGILTRRTFPLSSDEI | CUCACCCGAUGGCA | UAAGAG | GCCCCU |
| | | LIPMAPDLRCFLPDRT | GCCUAGCAGCUUCA | CCACC | UGGGCC |
| | | NRLSALHQPQIRNAI | CCCUAUACAGAGAG | | UCCCCC |
| | | WKLARKKQNCLVKE | GUGUUCACCCAGUA | | CAGCCC |
| | | TFSSKFEKTRRNEYQ | CGGCGGCAGCAUCA | | CUCCUC |
| | | RFLKKGGSVKSVAD | ACAUGCACCCGGAC | | CCCUUC |
| | | CSSDELTHIFIELFRSR | AUCGUGGACUACUU | | CUGCAC |
| | | FGNTASCYPADNLAN | CAUGAAGAGACACA | | CCGUAC |
| | | FFSQLHHLLFGHILYI | ACUGGCACUUCAAG | | CCCCGU |
| | | EGIPCAFDIVLKSESQ | UUCUUCCACUACAA | | GGUCUU |
| | | MNVYFDVSNGAIKN | GGAGGACGACAAGA | | UGAAUA |
| | | ECRPLSPGSILMWLNI | UCAAGGGCGCGUAU | | AAGUCU |
| | | ARARHYCQERQKKL | UUCAUCUGCAACGA | | GAGUGG |
| | | LFSIGILKPEWEYKR | CCAGAACAUCGGCA | | GCGGC |
| | | MWSTPYFTGKSICHH | UCCUAACACGAAGA | | |
| | | HHHH | ACCUUCCCUCUGAG | | |
| | | | CAGCGACGAGAUCC | | |
| | | | UGAUCCCUAUGGCC | | |
| | | | CCUGACCUGAGAUG | | |
| | | | CUUCCUGCCUGACA | | |
| | | | GAACCAACAGACUG | | |
| | | | AGCGCCCUGCACCA | | |
| | | | GCCUCAGAUCAGAA | | |
| | | | ACGCCAUCUGGAAG | | |
| | | | CUGGCCAGAAAGAA | | |
| | | | GCAGAACUGCCUGG | | |
| | | | UGAAGGAAACGUUC | | |
| | | | AGCAGCAAGUUCGA | | |
| | | | GAAGACUCGGCGCA | | |
| | | | ACGAGUACCAGAGA | | |
| | | | UUCCUGAAGAAGGG | | |
| | | | AGGUAGCGUCAAGA | | |
| | | | GCGUGGCCGACUGC | | |
| | | | AGUUCGGACGAACU | | |
| | | | GACCCACAUCUUCA | | |
| | | | UCGAGCUGUUCCGA | | |
| | | | UCCAGAUUCGGCAA | | |
| | | | CACCGCCAGCUGCU | | |
| | | | ACCCUGCCGACAAC | | |
| | | | CUGGCCAACUUCUU | | |
| | | | CAGCCAGCUGCACC | | |
| | | | ACCUGCUGUUCGGC | | |
| | | | CACAUCCUGUACAU | | |
| | | | CGAGGGCAUCCCUU | | |
| | | | GCGCCUUCGACAUC | | |
| | | | GUUUUGAAGAGUG | | |
| | | | AGAGCCAGAUGAAC | | |
| | | | GUGUACUUCGACGU | | |
| | | | GAGCAACGGCGCCA | | |
| | | | UCAAGAACGAGUGC | | |
| | | | AGACCGUUGUCCCC | | |
| | | | UGGAUCUAUCCUUA | | |
| | | | UGUGGCUGAACAUC | | |
| | | | GCCCGGGCUCGCCA | | |
| | | | CUACUGCCAGGAGA | | |
| | | | GACAGAAGAAGCUU | | |
| | | | CUCUUCUCAAUCGG | | |
| | | | AAUCUUAAAGCCUG | | |
| | | | AGUGGGAGUACAAG | | |
| | | | AGAAUGUGGAGCAC | | |
| | | | CCCUUACUUCACCG | | |
| | | | GCAAGAGCAUCUGC | | |
| | | | CACCACCAUCAUCA | | |
| | | | CCAC | | |
| SEQ ID NO: | | 71 | 72 | 3 | 4 |

SEQ ID NO: 195 consists of from 5′ end to 3′ end, 5′ UTR SEQ
ID NO: 3, mRNA ORF SEQ ID NO: 72, and 3′ UTR SEQ ID NO: 4.

| | St_iroN_n | MGLQTTKWPSHGAF | AUGGGCCUGCAGAC | GGGAAA | UGAUAA |
|---|---|---|---|---|---|
| | FLRT2_c | FLKSWLIISLGLYSQV | CACAAAGUGGCCUU | UAAGAG | UAGGCU |
| | His | SKLLAAESTDDNGET | CUCACGGCGCAUUC | AGAAAA | GGAGCC |
| | | IVVESTAEQVLKQQP | UUUCUGAAGUCCUG | GAAGAG | UCGGUG |
| | | GVSIITRDDIQKNPPV | GCUGAUCAUCUCCC | UAAGAA | GCCAUG |
| | | NDLADIIRKMPGVNL | UGGGCCUGUACAGC | GAAAUA | CUUCUU |
| | | TSNSASGTRGNNRQI | CAGGUGUCCAAACU | UAAGAG | GCCCCU |
| | | DIRGMGPENTLVLID | GCUGGCCGCCGAGA | CCACC | UGGGCC |
| | | GVPVTSRNSVRYSW | GCACCGACGAUAAC | | UCCCCC |
| | | RGERDTRGDTNWVP | GGCGAGACAAUCGU | | CAGCCC |
| | | PEMVERIEMIRGPAA | GGUGGAAAGCACCG | | CUCCUC |
| | | ARYGSGAAGGVVNII | CCGAACAGGUGCUG | | CCCUUC |
| | | TKRPTNDWHGSLSLY | AAACAGCAGCCUGG | | CUGCAC |
| | | TNYPESSKEGDTRRG | CGUGUCCAUCAUCA | | CCGUAC |
| | | NFSLSGPLAGDTLTM | CCCGGGACGACAUC | | CCCCGU |
| | | RLYGNLNRTDADSW | CAGAAGAACCCUCC | | GGUCUU |
| | | DINSSAGTKNAAGRE | AGUCAACGACCUGG | | UGAAUA |
| | | GVTNKDINSVFSWK | CCGACAUCAUCAGA | | AAGUCU |
| | | MTPQQILDFEAGYSR | AAGAUGCCCGGCGU | | GAGUGG |
| | | QGNIYAGDTQNSNSN | GAACCUGACCAGCA | | GCGGC |
| | | AVTKSLAQSGRETNR | AUAGCGCCUCUGGC | | |
| | | LYRQNYGLTHNGIW | ACCCGGGGCAACAA | | |
| | | GWGQSRLGFYYEKT | CAGACAGAUCGACA | | |
| | | DNTRMNEGLSGGGE | UCAGAGGCAUGGGC | | |
| | | GRITNDQTFTTNRLTS | CCCGAGAAUACCCU | | |
| | | YRTSGEVNVPVIWLF | GGUGCUGAUUGAUG | | |
| | | EQTLTVGAEWNRDE | GCGUGCCCGUGACC | | |
| | | LNDPSSTSLTVKDSNI | AGCAGAAACAGCGU | | |
| | | AGIPGSAANRSSKNK | GCGGUAUUCUUGGA | | |
| | | SEISALYVEDNIEPMA | GAGGCGAGAGAGAC | | |
| | | GTNIIPGLRFDYLSES | ACCAGAGGCGACAC | | |
| | | GSNFSPSLNLSQELGE | CAAUUGGGUGCCAC | | |
| | | FVKVKAGIARAFKAP | CUGAGAUGGUGGAA | | |
| | | NLYQTSEGYLLYSKG | CGGAUCGAGAUGAU | | |
| | | NGCPKDITSGGCYLV | UAGAGGCCCCGCUG | | |
| | | GNKNLDPEISINKEIG | CCGCCAGAUAUGGA | | |
| | | LEFTVDDYHASVTYF | UCUGGUGCUGCUGG | | |
| | | RNDYQNKIVAGDQII | CGGCGUGGUCAAUA | | |
| | | GRSASGAYVLQWQN | UCAUCACCAAGAGG | | |
| | | GGKALIEGIEASMAV | CCCACCAACGACUG | | |
| | | PLMPDRLNWNTNAT | GCACGGCAGCCUGA | | |
| | | YMITSEQKDTGNPLSI | GCCUGUAUACAAAC | | |
| | | IPKYTVNTFLDWTIT | UACCCCGAGAGCAG | | |
| | | NALSANVNWTLYGK | CAAAGAGGGCGAUA | | |
| | | QKPRTHAESRSEETK | CCAGAAGAGGCAAC | | |
| | | GLSGKALGAYSLVG | UUUAGCCUGUCUGG | | |
| | | ANVNYDINKNLRLN | CCCUCUGGCCGGCG | | |
| | | VGISNIFDKQIYRSAE | AUACCCUGACAAUG | | |
| | | GANTYNEPGRAYYA | AGACUGUACGGCAA | | |
| | | GVTASFHHHHHH | CCUGAACCGGACCG | | |
| | | | ACGCCGAUAGCUGG | | |
| | | | GACAUCAAUAGCAG | | |
| | | | CGCCGGCACCAAGA | | |
| | | | AUGCCGCCGGAAGA | | |
| | | | GAAGGCGUGACCAA | | |
| | | | CAAGGACAUCAACA | | |
| | | | GCGUGUUCAGCUGG | | |
| | | | AAGAUGACCCCUCA | | |
| | | | GCAGAUCCUGGAUU | | |
| | | | UCGAGGCCGGCUAU | | |
| | | | AGCAGACAGGGCAA | | |
| | | | CAUCUAUGCCGGCG | | |
| | | | ACACCCAGAACAGC | | |
| | | | AACAGCAACGCCGU | | |
| | | | GACCAAGUCUCUGG | | |
| | | | CCCAGUCUGGCAGA | | |
| | | | GAGACAAACAGGCU | | |
| | | | GUACCGGCAGAACU | | |
| | | | ACGGCCUGACACAC | | |
| | | | AAUGGCAUCUGGGG | | |
| | | | CUGGGGACAGUCUA | | |
| | | | GGCUGGGCUUCUAC | | |
| | | | UACGAGAAGACCGA | | |
| | | | CAACACCCGGAUGA | | |
| | | | ACGAGGGACUUUCU | | |
| | | | GGCGGCGGAGAGGG | | |
| | | | CAGAAUCACCAACG | | |
| | | | AUCAGACCUUCACC | | |
| | | | ACCAACCGGCUGAC | | |
| | | | CAGCUACAGAACCA | | |
| | | | GCGGCGAAGUGAAC | | |
| | | | GUGCCAGUGAUCUG | | |
| | | | GCUGUUCGAGCAGA | | |
| | | | CCCUGACAGUGGGC | | |
| | | | GCCGAGUGGAAUAG | | |
| | | | AGAUGAGCUGAACG | | |
| | | | ACCCCAGCUCCACC | | |
| | | | AGCCUGACCGUGAA | | |
| | | | GGACUCUAAUAUCG | | |
| | | | CUGGCAUCCCUGGC | | |
| | | | AGCGCCGCCAACAG | | |
| | | | AAGCAGCAAGAACA | | |
| | | | AGAGCGAGAUCAGC | | |
| | | | GCCCUGUACGUCGA | | |
| | | | GGACAACAUCGAAC | | |
| | | | CUAUGGCCGGCACA | | |
| | | | AACAUCAUCCCCGG | | |
| | | | CCUGAGAUUCGACU | | |
| | | | ACCUGAGCGAGAGC | | |
| | | | GGCAGCAACUUCAG | | |
| | | | CCCCAGCCUGAAUC | | |
| | | | UGUCUCAAGAGCUG | | |
| | | | GGCGAGUUCGUGAA | | |
| | | | AGUGAAGGCCGGAA | | |
| | | | UCGCCAGAGCCUUC | | |
| | | | AAGGCCCCUAAUCU | | |
| | | | GUACCAGACCAGCG | | |
| | | | AGGGCUACCUGCUG | | |
| | | | UACUCCAAAGGCAA | | |
| | | | CGGCUGCCCCAAGG | | |
| | | | AUAUCACAAGCGGC | | |
| | | | GGCUGUUACCUCGU | | |
| | | | GGGCAACAAGAACC | | |
| | | | UGGAUCCUGAGAUC | | |
| | | | AGCAUCAACAAAGA | | |
| | | | GAUCGGCCUGGAAU | | |
| | | | UCACCGUGGACGAC | | |
| | | | UACCACGCCAGCGU | | |
| | | | GACCUACUUCCGGA | | |
| | | | ACGAUUACCAGAAC | | |
| | | | AAGAUCGUGGCCGG | | |
| | | | GGACCAGAUCAUCG | | |
| | | | GCAGAAGUGCUAGC | | |
| | | | GGAGCCUACGUGCU | | |
| | | | GCAAUGGCAGAAUG | | |
| | | | GCGGAAAGGCCCUG | | |
| | | | AUCGAGGGAAUCGA | | |
| | | | GGCUUCUAUGGCCG | | |
| | | | UGCCACUGAUGCCC | | |
| | | | GACAGACUGAACUG | | |
| | | | GAACACCAACGCCA | | |
| | | | CCUACAUGAUCACC | | |
| | | | AGCGAGCAGAAGGA | | |
| | | | CACCGGCAAUCCUC | | |
| | | | UGAGCAUCAUCCCU | | |
| | | | AAGUAUACCGUGAA | | |
| | | | CACCUUCCUGGACU | | |
| | | | GGACCAUCACAAAC | | |
| | | | GCCCUGAGCGCCAA | | |
| | | | CGUGAACUGGACCC | | |
| | | | UGUAUGGCAAGCAG | | |
| | | | AAGCCACGGACACA | | |
| | | | CGCCGAGUCCAGAA | | |
| | | | GCGAGGAAACAAAG | | |
| | | | GGCCUGAGCGGCAA | | |
| | | | AGCCCUGGGCGCCU | | |
| | | | AUUCUCUUGUGGGC | | |
| | | | GCCAAUGUGAAUUA | | |
| | | | CGACAUUAACAAGA | | |
| | | | AUCUGCGGCUGAAC | | |
| | | | GUGGGCAUCAGCAA | | |
| | | | CAUCUUCGACAAGC | | |
| | | | AGAUCUACAGAAGC | | |
| | | | GCCGAGGGCGCCAA | | |
| | | | CACCUACAAUGAAC | | |
| | | | CUGGCAGAGCCUAC | | |
| | | | UAUGCUGGCGUGAC | | |
| | | | CGCCAGCUUCCAUC | | |
| | | | ACCACCAUCAUCAU | | |
| SEQ ID NO: | | 73 | 74 | 3 | 4 |

SEQ ID NO: 196 consists of from 5′ end to 3′ end, 5′ UTR SEQ
ID NO: 3, mRNA ORF SEQ ID NO: 74, and 3′ UTR SEQ ID NO: 4.

| | St_iroN_n | MGLQTTKWPSHGAF | AUGGGCCUGCAGAC | GGGAAA | UGAUAA |
|---|---|---|---|---|---|
| | FLRT2 | FLKSWLIISLGLYSQV | CACAAAGUGGCCUU | UAAGAG | UAGGCU |
| | | SKLLAAESTDDNGET | CUCACGGCGCAUUC | AGAAAA | GGAGCC |
| | | IVVESTAEQVLKQQP | UUUCUGAAGUCCUG | GAAGAG | UCGGUG |
| | | GVSIITRDDIQKNPPV | GCUGAUCAUCUCCC | UAAGAA | GCCAUG |
| | | NDLADIIRKMPGVNL | UGGGCCUGUACAGC | GAAAUA | CUUCUU |
| | | TSNSASGTRGNNRQI | CAGGUGUCCAAACU | UAAGAG | GCCCCU |
| | | GVPVTSRNSVRYSW | GCACCGACGAUAAC | | UCCCCC |
| | | RGERDTRGDTNWVP | GGCGAGACAAUCGU | | CAGCCC |
| | | PEMVERIEMIRGPAA | GGUGGAAAGCACCG | | CUCCUC |
| | | ARYGSGAAGGVVNII | CCGAACAGGUGCUG | | CCCUUC |
| | | TKRPTNDWHGSLSLY | AAACAGCAGCCUGG | | CUGCAC |
| | | TNYPESSKEGDTRRG | CGUGUCCAUCAUCA | | CCGUAC |
| | | NFSLSGPLAGDTLTM | CCCGGGACGACAUC | | CCCCGU |
| | | RLYGNLNRTDADSW | CAGAAGAACCCUCC | | GGUCUU |
| | | DINSSAGTKNAAGRE | AGUCAACGACCUGG | | UGAAUA |
| | | GVTNKDINSVFSWK | CCGACAUCAUCAGA | | AAGUCU |
| | | MTPQQILDFEAGYSR | AAGAUGCCCGGCGU | | GAGUGG |
| | | QGNIYAGDTQNSNSN | GAACCUGACCAGCA | | GCGGC |
| | | AVTKSLAQSGRETNR | AUAGCGCCUCUGGC | | |
| | | LYRQNYGLTHNGIW | ACCCGGGGCAACAA | | |
| | | GWGQSRLGFYYEKT | CAGACAGAUCGACA | | |
| | | DNTRMNEGLSGGGE | UCAGAGGCAUGGGC | | |
| | | GRITNDQTFTTNRLTS | CCCGAGAAUACCCU | | |
| | | YRTSGEVNVPVIWLF | GGUGCUGAUUGAUG | | |
| | | EQTLTVGAEWNRDE | GCGUGCCCGUGACC | | |
| | | LNDPSSTSLTVKDSNI | AGCAGAAACAGCGU | | |
| | | AGIPGSAANRSSKNK | GCGGUAUUCUUGGA | | |
| | | SEISALYVEDNIEPMA | GAGGCGAGAGAGAC | | |
| | | GTNIIPGLRFDYLSES | ACCAGAGGCGACAC | | |
| | | GSNFSPSLNLSQELGE | CAAUUGGGUGCCAC | | |
| | | FVKVKAGIARAFKAP | CUGAGAUGGUGGAA | | |
| | | NLYQTSEGYLLYSKG | CGGAUCGAGAUGAU | | |
| | | NGCPKDITSGGCYLV | UAGAGGCCCCGCUG | | |
| | | GNKNLDPEISINKEIG | CCGCCAGAUAUGGA | | |
| | | LEFTVDDYHASVTYF | UCUGGUGCUGCUGG | | |
| | | RNDYQNKIVAGDQII | CGGCGUGGUCAAUA | | |
| | | GRSASGAYVLQWQN | UCAUCACCAAGAGG | | |
| | | GGKALIEGIEASMAV | CCCACCAACGACUG | | |
| | | PLMPDRLNWNTNAT | GCACGGCAGCCUGA | | |
| | | YMITSEQKDTGNPLSI | GCCUGUAUACAAAC | | |
| | | IPKYTVNTFLDWTIT | UACCCCGAGAGCAG | | |
| | | NALSANVNWTLYGK | CAAAGAGGGCGAUA | | |
| | | QKPRTHAESRSEETK | CCAGAAGAGGCAAC | | |
| | | GLSGKALGAYSLVG | UUUAGCCUGUCUGG | | |
| | | ANVNYDINKNLRLN | CCCUCUGGCCGGCG | | |
| | | VGISNIFDKQIYRSAE | AUACCCUGACAAUG | | |
| | | GANTYNEPGRAYYA | AGACUGUACGGCAA | | |
| | | GVTASF | CCUGAACCGGACCG | | |
| | | | ACGCCGAUAGCUGG | | |
| | | | GACAUCAAUAGCAG | | |
| | | | CGCCGGCACCAAGA | | |
| | | | AUGCCGCCGGAAGA | | |
| | | | GAAGGCGUGACCAA | | |
| | | | CAAGGACAUCAACA | | |
| | | | GCGUGUUCAGCUGG | | |
| | | | AAGAUGACCCCUCA | | |
| | | | GCAGAUCCUGGAUU | | |
| | | | UCGAGGCCGGCUAU | | |
| | | | AGCAGACAGGGCAA | | |
| | | | CAUCUAUGCCGGCG | | |
| | | | ACACCCAGAACAGC | | |
| | | | AACAGCAACGCCGU | | |
| | | | GACCAAGUCUCUGG | | |
| | | | CCCAGUCUGGCAGA | | |
| | | | GAGACAAACAGGCU | | |
| | | | GUACCGGCAGAACU | | |
| | | | ACGGCCUGACACAC | | |
| | | | AAUGGCAUCUGGGG | | |
| | | | CUGGGGACAGUCUA | | |
| | | | GGCUGGGCUUCUAC | | |
| | | | UACGAGAAGACCGA | | |
| | | | CAACACCCGGAUGA | | |
| | | | ACGAGGGACUUUCU | | |
| | | | GGCGGCGGAGAGGG | | |
| | | | CAGAAUCACCAACG | | |
| | | | AUCAGACCUUCACC | | |
| | | | ACCAACCGGCUGAC | | |
| | | | CAGCUACAGAACCA | | |
| | | | GCGGCGAAGUGAAC | | |
| | | | GUGCCAGUGAUCUG | | |
| | | | GCUGUUCGAGCAGA | | |
| | | | CCCUGACAGUGGGC | | |
| | | | GCCGAGUGGAAUAG | | |
| | | | AGAUGAGCUGAACG | | |
| | | | ACCCCAGCUCCACC | | |
| | | | AGCCUGACCGUGAA | | |
| | | | GGACUCUAAUAUCG | | |
| | | | CUGGCAUCCCUGGC | | |
| | | | AGCGCCGCCAACAG | | |
| | | | AAGCAGCAAGAACA | | |
| | | | AGAGCGAGAUCAGC | | |
| | | | GCCCUGUACGUCGA | | |
| | | | GGACAACAUCGAAC | | |
| | | | CUAUGGCCGGCACA | | |
| | | | AACAUCAUCCCCGG | | |
| | | | CCUGAGAUUCGACU | | |
| | | | ACCUGAGCGAGAGC | | |
| | | | GGCAGCAACUUCAG | | |
| | | | CCCCAGCCUGAAUC | | |
| | | | UGUCUCAAGAGCUG | | |
| | | | GGCGAGUUCGUGAA | | |
| | | | AGUGAAGGCCGGAA | | |
| | | | UCGCCAGAGCCUUC | | |
| | | | AAGGCCCCUAAUCU | | |
| | | | GUACCAGACCAGCG | | |
| | | | AGGGCUACCUGCUG | | |
| | | | UACUCCAAAGGCAA | | |
| | | | CGGCUGCCCCAAGG | | |
| | | | AUAUCACAAGCGGC | | |
| | | | GGCUGUUACCUCGU | | |
| | | | GGGCAACAAGAACC | | |
| | | | UGGAUCCUGAGAUC | | |
| | | | AGCAUCAACAAAGA | | |
| | | | GAUCGGCCUGGAAU | | |
| | | | UCACCGUGGACGAC | | |
| | | | UACCACGCCAGCGU | | |
| | | | GACCUACUUCCGGA | | |
| | | | ACGAUUACCAGAAC | | |
| | | | AAGAUCGUGGCCGG | | |
| | | | GGACCAGAUCAUCG | | |
| | | | GCAGAAGUGCUAGC | | |
| | | | GGAGCCUACGUGCU | | |
| | | | GCAAUGGCAGAAUG | | |
| | | | GCGGAAAGGCCCUG | | |
| | | | AUCGAGGGAAUCGA | | |
| | | | GGCUUCUAUGGCCG | | |
| | | | UGCCACUGAUGCCC | | |
| | | | GACAGACUGAACUG | | |
| | | | GAACACCAACGCCA | | |
| | | | CCUACAUGAUCACC | | |
| | | | AGCGAGCAGAAGGA | | |
| | | | CACCGGCAAUCCUC | | |
| | | | UGAGCAUCAUCCCU | | |
| | | | AAGUAUACCGUGAA | | |
| | | | CACCUUCCUGGACU | | |
| | | | GGACCAUCACAAAC | | |
| | | | GCCCUGAGCGCCAA | | |
| | | | CGUGAACUGGACCC | | |
| | | | UGUAUGGCAAGCAG | | |
| | | | AAGCCACGGACACA | | |
| | | | CGCCGAGUCCAGAA | | |
| | | | GCGAGGAAACAAAG | | |
| | | | GGCCUGAGCGGCAA | | |
| | | | AGCCCUGGGCGCCU | | |
| | | | AUUCUCUUGUGGGC | | |
| | | | GCCAAUGUGAAUUA | | |
| | | | CGACAUUAACAAGA | | |
| | | | AUCUGCGGCUGAAC | | |
| | | | GUGGGCAUCAGCAA | | |
| | | | CAUCUUCGACAAGC | | |
| | | | AGAUCUACAGAAGC | | |
| | | | GCCGAGGGCGCCAA | | |
| | | | CACCUACAAUGAAC | | |
| | | | CUGGCAGAGCCUAC | | |
| | | | UAUGCUGGCGUGAC | | |
| | | | CGCCAGCUUC | | |
| SEQ ID NO: | | 75 | 76 | 3 | 4 |

SEQ ID NO: 197 consists of from 5′ end to 3′ end, 5′ UTR SEQ
ID NO: 3, mRNA ORF SEQ ID NO: 76, and 3′ UTR SEQ ID NO: 4.

| | St_iroN_N | MGLQTTKWPSHGAF | AUGGGCCUGCAGAC | GGGAAA | UGAUAA |
|---|---|---|---|---|---|
| | GM_nFLR | FLKSWLIISLGLYSQV | CACAAAGUGGCCUU | UAAGAG | UAGGCU |
| | T2_cHis | SKLLAAESTDDNGET | CUCACGGCGCAUUC | AGAAAA | GGAGCC |
| | | IVVESTAEQVLKQQP | UUUCUGAAGUCCUG | GAAGAG | UCGGUG |
| | | GVSIITRDDIQKNPPV | GCUGAUCAUCUCCC | UAAGAA | GCCAUG |
| | | NDLADIIRKMPGVNL | UGGGCCUGUACAGC | GAAAUA | CUUCUU |
| | | ASNSASGTRGNNRQI | CAGGUGUCCAAACU | UAAGAG | GCCCCU |
| | | DIRGMGPENTLVLID | GCUGGCCGCCGAGA | CCACC | UGGGCC |
| | | GVPVTSRNSVRYSW | GCACCGACGAUAAC | | UCCCCC |
| | | RGERDTRGDTNWVP | GGCGAGACAAUCGU | | CAGCCC |
| | | PEMVERIEMIRGPAA | GGUGGAAAGCACCG | | CUCCUC |
| | | ARYGSGAAGGVVNII | CCGAACAGGUGCUG | | CCCUUC |
| | | TKRPTNDWHGSLSLY | AAACAGCAGCCUGG | | CUGCAC |
| | | TNYPESSKEGDTRRG | CGUGUCCAUCAUCA | | CCGUAC |
| | | NFALSGPLAGDTLTM | CCCGGGACGACAUC | | CCCCGU |
| | | RLYGNLNRADADSW | CAGAAGAACCCUCC | | GGUCUU |
| | | DINSSAGTKNAAGRE | AGUCAACGACCUGG | | UGAAUA |
| | | GVTNKDINSVFSWK | CCGACAUCAUCAGA | | AAGUCU |
| | | MTPQQILDFEAGYSR | AAGAUGCCCGGCGU | | GAGUGG |
| | | QGNIYAGDTQNSNSN | GAACCUGGCCAGCA | | GCGGC |
| | | AVTKSLAQSGRETNR | AUAGCGCCUCUGGC | | |
| | | LYRQNYGLTHNGIW | ACCCGGGGCAACAA | | |
| | | GWGQSRLGFYYEKT | CAGACAGAUCGACA | | |
| | | DNTRMNEGLSGGGE | UCAGAGGCAUGGGC | | |
| | | GRITNDQTFTTNRLTS | CCCGAGAAUACCCU | | |
| | | YRTSGEVNVPVIWLF | GGUGCUGAUUGAUG | | |
| | | EQTLTVGAEWNRDE | GCGUGCCCGUGACC | | |
| | | LNDPSSTSLTVKDSNI | AGCAGAAACAGCGU | | |
| | | AGIPGSAANRASKNK | GCGGUAUUCUUGGA | | |
| | | SEISALYVEDNIEPMA | GAGGCGAGAGAGAC | | |
| | | GTNIIPGLRFDYLSES | ACCAGAGGCGACAC | | |
| | | GSNFSPSLNLAQELG | CAAUUGGGUGCCAC | | |
| | | EFVKVKAGIARAFKA | CUGAGAUGGUGGAA | | |
| | | PNLYQTSEGYLLYSK | CGGAUCGAGAUGAU | | |
| | | GNGCPKDITSGGCYL | UAGAGGCCCCGCUG | | |
| | | VGNKNLDPEISINKEI | CCGCCAGAUAUGGA | | |
| | | GLEFTVDDYHASVTY | UCUGGUGCUGCUGG | | |
| | | FRNDYQNKIVAGDQI | CGGCGUGGUCAAUA | | |
| | | IGRSASGAYVLQWQ | UCAUCACCAAGAGG | | |
| | | NGGKALIEGIEASMA | CCCACCAACGACUG | | |
| | | VPLMPDRLNWNTNA | GCACGGCAGCCUGA | | |
| | | AYMITSEQKDTGNPL | GCCUGUAUACAAAC | | |
| | | SIIPKYTVNTFLDWTI | UACCCCGAGAGCAG | | |
| | | TNALSANVNWTLYG | CAAAGAGGGCGAUA | | |
| | | KQKPRTHAESRSEET | CCAGAAGAGGCAAC | | |
| | | KGLSGKALGAYSLV | UUUGCCCUGUCUGG | | |
| | | GANVNYDINKNLRL | CCCUCUGGCCGGCG | | |
| | | NVGISNIFDKQIYRSA | AUACCCUGACAAUG | | |
| | | EGANTYNEPGRAYY | AGACUGUACGGCAA | | |
| | | AGVTASFHHHHHH | CCUGAACCGGGCCG | | |
| | | | ACGCCGAUAGCUGG | | |
| | | | GACAUCAAUAGCAG | | |
| | | | CGCCGGCACCAAGA | | |
| | | | AUGCCGCCGGAAGA | | |
| | | | GAAGGCGUGACCAA | | |
| | | | CAAGGACAUCAACA | | |
| | | | GCGUGUUCAGCUGG | | |
| | | | AAGAUGACCCCUCA | | |
| | | | GCAGAUCCUGGAUU | | |
| | | | UCGAGGCCGGCUAU | | |
| | | | AGCAGACAGGGCAA | | |
| | | | CAUCUAUGCCGGCG | | |
| | | | ACACCCAGAACAGC | | |
| | | | AACAGCAACGCCGU | | |
| | | | GACCAAGUCUCUGG | | |
| | | | CCCAGUCUGGCAGA | | |
| | | | GAGACAAACAGGCU | | |
| | | | GUACCGGCAGAACU | | |
| | | | ACGGCCUGACACAC | | |
| | | | AAUGGCAUCUGGGG | | |
| | | | CUGGGGACAGUCUA | | |
| | | | GGCUGGGCUUCUAC | | |
| | | | UACGAGAAGACCGA | | |
| | | | CAACACCCGGAUGA | | |
| | | | ACGAGGGACUUUCU | | |
| | | | GGCGGCGGAGAGGG | | |
| | | | CAGAAUCACCAACG | | |
| | | | AUCAGACCUUCACC | | |
| | | | ACCAACCGGCUGAC | | |
| | | | CAGCUACAGAACCA | | |
| | | | GCGGCGAAGUGAAC | | |
| | | | GUGCCAGUGAUCUG | | |
| | | | GCUGUUCGAGCAGA | | |
| | | | CCCUGACAGUGGGC | | |
| | | | GCCGAGUGGAAUAG | | |
| | | | AGAUGAGCUGAACG | | |
| | | | ACCCCAGCUCCACC | | |
| | | | AGCCUGACCGUGAA | | |
| | | | GGACUCUAAUAUCG | | |
| | | | CUGGCAUCCCUGGC | | |
| | | | AGCGCCGCCAACAG | | |
| | | | AGCCAGCAAGAACA | | |
| | | | AGAGCGAGAUCAGC | | |
| | | | GCCCUGUACGUCGA | | |
| | | | GGACAACAUCGAAC | | |
| | | | CUAUGGCCGGCACA | | |
| | | | AACAUCAUCCCCGG | | |
| | | | CCUGAGAUUCGACU | | |
| | | | ACCUGAGCGAGAGC | | |
| | | | GGCAGCAACUUCAG | | |
| | | | CCCCAGCCUGAAUC | | |
| | | | UGGCUCAAGAGCUG | | |
| | | | GGCGAGUUCGUGAA | | |
| | | | AGUGAAGGCCGGAA | | |
| | | | UCGCCAGAGCCUUC | | |
| | | | AAGGCCCCUAAUCU | | |
| | | | GUACCAGACCAGCG | | |
| | | | AGGGCUACCUGCUG | | |
| | | | UACUCCAAAGGCAA | | |
| | | | CGGCUGCCCCAAGG | | |
| | | | AUAUCACAAGCGGC | | |
| | | | GGCUGUUACCUCGU | | |
| | | | GGGCAACAAGAACC | | |
| | | | UGGAUCCUGAGAUC | | |
| | | | AGCAUCAACAAAGA | | |
| | | | GAUCGGCCUGGAAU | | |
| | | | UCACCGUGGACGAC | | |
| | | | UACCACGCCAGCGU | | |
| | | | GACCUACUUCCGGA | | |
| | | | ACGAUUACCAGAAC | | |
| | | | AAGAUCGUGGCCGG | | |
| | | | GGACCAGAUCAUCG | | |
| | | | GCAGAAGUGCUAGC | | |
| | | | GGAGCCUACGUGCU | | |
| | | | GCAAUGGCAGAAUG | | |
| | | | GCGGAAAGGCCCUG | | |
| | | | AUCGAGGGAAUCGA | | |
| | | | GGCUUCUAUGGCCG | | |
| | | | UGCCACUGAUGCCC | | |
| | | | GACAGACUGAACUG | | |
| | | | GAACACCAACGCCG | | |
| | | | CCUACAUGAUCACC | | |
| | | | AGCGAGCAGAAGGA | | |
| | | | CACCGGCAAUCCUC | | |
| | | | UGAGCAUCAUCCCU | | |
| | | | AAGUAUACCGUGAA | | |
| | | | CACCUUCCUGGACU | | |
| | | | GGACCAUCACAAAC | | |
| | | | GCCCUGAGCGCCAA | | |
| | | | CGUGAACUGGACCC | | |
| | | | UGUAUGGCAAGCAG | | |
| | | | AAGCCACGGACACA | | |
| | | | CGCCGAGUCCAGAA | | |
| | | | GCGAGGAAACAAAG | | |
| | | | GGCCUGAGCGGCAA | | |
| | | | AGCCCUGGGCGCCU | | |
| | | | AUUCUCUUGUGGGC | | |
| | | | GCCAAUGUGAAUUA | | |
| | | | CGACAUUAACAAGA | | |
| | | | AUCUGCGGCUGAAC | | |
| | | | GUGGGCAUCAGCAA | | |
| | | | CAUCUUCGACAAGC | | |
| | | | AGAUCUACAGAAGC | | |
| | | | GCCGAGGGCGCCAA | | |
| | | | CACCUACAAUGAAC | | |
| | | | CUGGCAGAGCCUAC | | |
| | | | UAUGCUGGCGUGAC | | |
| | | | CGCCAGCUUCCAUC | | |
| | | | ACCACCAUCAUCAU | | |
| SEQ ID NO: | | 77 | 78 | 3 | 4 |

SEQ ID NO: 198 consists of from 5′ end to 3′ end, 5′ UTR SEQ
ID NO: 3, mRNA ORF SEQ ID NO: 78, and 3′ UTR SEQ ID NO: 4.

| | St_iroN_N | MGLQTTKWPSHGAF | AUGGGCCUGCAGAC | GGGAAA | UGAUAA |
|---|---|---|---|---|---|
| | GM_nFLR | FLKSWLIISLGLYSQV | CACAAAGUGGCCUU | UAAGAG | UAGGCU |
| | T2 | SKLLAAESTDDNGET | CUCACGGCGCAUUC | AGAAAA | GGAGCC |
| | | IVVESTAEQVLKQQP | UUUCUGAAGUCCUG | GAAGAG | UCGGUG |
| | | GVSIITRDDIQKNPPV | GCUGAUCAUCUCCC | UAAGAA | GCCAUG |
| | | NDLADIIRKMPGVNL | UGGGCCUGUACAGC | GAAAUA | CUUCUU |
| | | ASNSASGTRGNNRQI | CAGGUGUCCAAACU | UAAGAG | GCCCCU |
| | | DIRGMGPENTLVLID | GCUGGCCGCCGAGA | CCACC | UGGGCC |
| | | GVPVTSRNSVRYSW | GCACCGACGAUAAC | | UCCCCC |
| | | RGERDTRGDTNWVP | GGCGAGACAAUCGU | | CAGCCC |
| | | PEMVERIEMIRGPAA | GGUGGAAAGCACCG | | CUCCUC |
| | | ARYGSGAAGGVVNII | CCGAACAGGUGCUG | | CCCUUC |
| | | TKRPTNDWHGSLSLY | AAACAGCAGCCUGG | | CUGCAC |
| | | TNYPESSKEGDTRRG | CGUGUCCAUCAUCA | | CCGUAC |
| | | NFALSGPLAGDTLTM | CCCGGGACGACAUC | | CCCCGU |
| | | RLYGNLNRADADSW | CAGAAGAACCCUCC | | GGUCUU |
| | | DINSSAGTKNAAGRE | AGUCAACGACCUGG | | UGAAUA |
| | | GVTNKDINSVFSWK | CCGACAUCAUCAGA | | AAGUCU |
| | | MTPQQILDFEAGYSR | AAGAUGCCCGGCGU | | GAGUGG |
| | | QGNIYAGDTQNSNSN | GAACCUGGCCAGCA | | GCGGC |
| | | AVTKSLAQSGRETNR | AUAGCGCCUCUGGC | | |
| | | LYRQNYGLTHNGIW | ACCCGGGGCAACAA | | |
| | | GWGQSRLGFYYEKT | CAGACAGAUCGACA | | |
| | | DNTRMNEGLSGGGE | UCAGAGGCAUGGGC | | |
| | | GRITNDQTFTTNRLTS | CCCGAGAAUACCCU | | |
| | | YRTSGEVNVPVIWLF | GGUGCUGAUUGAUG | | |
| | | EQTLTVGAEWNRDE | GCGUGCCCGUGACC | | |
| | | LNDPSSTSLTVKDSNI | AGCAGAAACAGCGU | | |
| | | AGIPGSAANRASKNK | GCGGUAUUCUUGGA | | |
| | | SEISALYVEDNIEPMA | GAGGCGAGAGAGAC | | |
| | | GTNIIPGLRFDYLSES | ACCAGAGGCGACAC | | |
| | | GSNFSPSLNLAQELG | CAAUUGGGUGCCAC | | |
| | | EFVKVKAGIARAFKA | CUGAGAUGGUGGAA | | |
| | | PNLYQTSEGYLLYSK | CGGAUCGAGAUGAU | | |
| | | GNGCPKDITSGGCYL | UAGAGGCCCCGCUG | | |
| | | VGNKNLDPEISINKEI | CCGCCAGAUAUGGA | | |
| | | GLEFTVDDYHASVTY | UCUGGUGCUGCUGG | | |
| | | FRNDYQNKIVAGDQI | CGGCGUGGUCAAUA | | |
| | | IGRSASGAYVLQWQ | UCAUCACCAAGAGG | | |
| | | NGGKALIEGIEASMA | CCCACCAACGACUG | | |
| | | VPLMPDRLNWNTNA | GCACGGCAGCCUGA | | |
| | | AYMITSEQKDTGNPL | GCCUGUAUACAAAC | | |
| | | SIIPKYTVNTFLDWTI | UACCCCGAGAGCAG | | |
| | | TNALSANVNWTLYG | CAAAGAGGGCGAUA | | |
| | | KQKPRTHAESRSEET | CCAGAAGAGGCAAC | | |
| | | KGLSGKALGAYSLV | UUUGCCCUGUCUGG | | |
| | | GANVNYDINKNLRL | CCCUCUGGCCGGCG | | |
| | | NVGISNIFDKQIYRSA | AUACCCUGACAAUG | | |
| | | EGANTYNEPGRAYY | AGACUGUACGGCAA | | |
| | | AGVTASF | CCUGAACCGGGCCG | | |
| | | | ACGCCGAUAGCUGG | | |
| | | | GACAUCAAUAGCAG | | |
| | | | CGCCGGCACCAAGA | | |
| | | | AUGCCGCCGGAAGA | | |
| | | | GAAGGCGUGACCAA | | |
| | | | CAAGGACAUCAACA | | |
| | | | GCGUGUUCAGCUGG | | |
| | | | AAGAUGACCCCUCA | | |
| | | | GCAGAUCCUGGAUU | | |
| | | | UCGAGGCCGGCUAU | | |
| | | | AGCAGACAGGGCAA | | |
| | | | CAUCUAUGCCGGCG | | |
| | | | ACACCCAGAACAGC | | |
| | | | AACAGCAACGCCGU | | |
| | | | GACCAAGUCUCUGG | | |
| | | | CCCAGUCUGGCAGA | | |
| | | | GAGACAAACAGGCU | | |
| | | | GUACCGGCAGAACU | | |
| | | | ACGGCCUGACACAC | | |
| | | | AAUGGCAUCUGGGG | | |
| | | | CUGGGGACAGUCUA | | |
| | | | GGCUGGGCUUCUAC | | |
| | | | UACGAGAAGACCGA | | |
| | | | CAACACCCGGAUGA | | |
| | | | ACGAGGGACUUUCU | | |
| | | | GGCGGCGGAGAGGG | | |
| | | | CAGAAUCACCAACG | | |
| | | | AUCAGACCUUCACC | | |
| | | | ACCAACCGGCUGAC | | |
| | | | CAGCUACAGAACCA | | |
| | | | GCGGCGAAGUGAAC | | |
| | | | GUGCCAGUGAUCUG | | |
| | | | GCUGUUCGAGCAGA | | |
| | | | CCCUGACAGUGGGC | | |
| | | | GCCGAGUGGAAUAG | | |
| | | | AGAUGAGCUGAACG | | |
| | | | ACCCCAGCUCCACC | | |
| | | | AGCCUGACCGUGAA | | |
| | | | GGACUCUAAUAUCG | | |
| | | | CUGGCAUCCCUGGC | | |
| | | | AGCGCCGCCAACAG | | |
| | | | AGCCAGCAAGAACA | | |
| | | | AGAGCGAGAUCAGC | | |
| | | | GCCCUGUACGUCGA | | |
| | | | GGACAACAUCGAAC | | |
| | | | CUAUGGCCGGCACA | | |
| | | | AACAUCAUCCCCGG | | |
| | | | CCUGAGAUUCGACU | | |
| | | | ACCUGAGCGAGAGC | | |
| | | | GGCAGCAACUUCAG | | |
| | | | CCCCAGCCUGAAUC | | |
| | | | UGGCUCAAGAGCUG | | |
| | | | GGCGAGUUCGUGAA | | |
| | | | AGUGAAGGCCGGAA | | |
| | | | UCGCCAGAGCCUUC | | |
| | | | AAGGCCCCUAAUCU | | |
| | | | GUACCAGACCAGCG | | |
| | | | AGGGCUACCUGCUG | | |
| | | | UACUCCAAAGGCAA | | |
| | | | CGGCUGCCCCAAGG | | |
| | | | AUAUCACAAGCGGC | | |
| | | | GGCUGUUACCUCGU | | |
| | | | GGGCAACAAGAACC | | |
| | | | UGGAUCCUGAGAUC | | |
| | | | AGCAUCAACAAAGA | | |
| | | | GAUCGGCCUGGAAU | | |
| | | | UCACCGUGGACGAC | | |
| | | | UACCACGCCAGCGU | | |
| | | | GACCUACUUCCGGA | | |
| | | | ACGAUUACCAGAAC | | |
| | | | AAGAUCGUGGCCGG | | |
| | | | GGACCAGAUCAUCG | | |
| | | | GCAGAAGUGCUAGC | | |
| | | | GGAGCCUACGUGCU | | |
| | | | GCAAUGGCAGAAUG | | |
| | | | GCGGAAAGGCCCUG | | |
| | | | AUCGAGGGAAUCGA | | |
| | | | GGCUUCUAUGGCCG | | |
| | | | UGCCACUGAUGCCC | | |
| | | | GACAGACUGAACUG | | |
| | | | GAACACCAACGCCG | | |
| | | | CCUACAUGAUCACC | | |
| | | | AGCGAGCAGAAGGA | | |
| | | | CACCGGCAAUCCUC | | |
| | | | UGAGCAUCAUCCCU | | |
| | | | AAGUAUACCGUGAA | | |
| | | | CACCUUCCUGGACU | | |
| | | | GGACCAUCACAAAC | | |
| | | | GCCCUGAGCGCCAA | | |
| | | | CGUGAACUGGACCC | | |
| | | | UGUAUGGCAAGCAG | | |
| | | | AAGCCACGGACACA | | |
| | | | CGCCGAGUCCAGAA | | |
| | | | GCGAGGAAACAAAG | | |
| | | | GGCCUGAGCGGCAA | | |
| | | | AGCCCUGGGCGCCU | | |
| | | | AUUCUCUUGUGGGC | | |
| | | | GCCAAUGUGAAUUA | | |
| | | | CGACAUUAACAAGA | | |
| | | | AUCUGCGGCUGAAC | | |
| | | | GUGGGCAUCAGCAA | | |
| | | | CAUCUUCGACAAGC | | |
| | | | AGAUCUACAGAAGC | | |
| | | | GCCGAGGGCGCCAA | | |
| | | | CACCUACAAUGAAC | | |
| | | | CUGGCAGAGCCUAC | | |
| | | | UAUGCUGGCGUGAC | | |
| | | | CGCCAGCUUC | | |
| SEQ ID NO: | | 79 | 80 | 3 | 4 |

SEQ ID NO: 199 consists of from 5′ end to 3′ end, 5′ UTR SEQ
ID NO: 3, mRNA ORF SEQ ID NO: 80, and 3′ UTR SEQ ID NO: 4.

| | St_CirA_n | MGLQTTKWPSHGAF | AUGGGCCUGCAGAC | GGGAAA | UGAUAA |
|---|---|---|---|---|---|
| | FLRT2_c | FLKSWLIISLGLYSQV | CACAAAGUGGCCUU | UAAGAG | UAGGCU |
| | His | SKLLAATDDGETMV | CUCACGGCGCAUUC | AGAAAA | GGAGCC |
| | | VTASAIEQNLKDAPA | UUUCUGAAGUCCUG | GAAGAG | UCGGUG |
| | | SISVITQQDLQRRPVQ | GCUGAUCAUCUCCC | UAAGAA | GCCAUG |
| | | NLKDVLKEVPGVQL | UGGGCCUGUACAGC | GAAAUA | CUUCUU |
| | | TNEGDNRKGVSIRGL | CAGGUGUCCAAACU | UAAGAG | GCCCCU |
| | | DSSYTLILIDGKRVNS | GCUGGCCGCCACCG | CCACC | UGGGCC |
| | | RNAVFRHNDFDLNW | AUGAUGGCGAGACA | | UCCCCC |
| | | IPVDAIERIEVVRGPM | AUGGUGGUUACAGC | | CAGCCC |
| | | SSLYGSDALGGVVNII | CAGCGCCAUCGAGC | | CUCCUC |
| | | TKKIGQKWHGSVTV | AGAACCUGAAAGAU | | CCCUUC |
| | | DSTIQEHRDRGDTYN | GCCCCUGCCAGCAU | | CUGCAC |
| | | GQFFTSGPLIDGVLG | CAGCGUGAUCACCC | | CCGUAC |
| | | MKAYGSLAKREKDE | AGCAGGAUCUGCAG | | CCCCGU |
| | | QQSSATTATGETPRIE | AGAAGGCCCGUGCA | | GGUCUU |
| | | GFTSRDGNVEFAWTP | GAAUCUGAAGGACG | | UGAAUA |
| | | NENHDVTAGYGFDR | UGCUGAAAGAGGUG | | AAGUCU |
| | | QDRDSDSLDKNRLER | CCCGGCGUCCAGCU | | GAGUGG |
| | | QNYALSHNGRWDLG | GACAAACGAGGGCG | | GCGGC |
| | | NSELKFYGEKVENKN | AUAAUAGAAAGGGC | | |
| | | PGNSSPITSESNSIDG | GUGUCCAUCAGAGG | | |
| | | KYVLPLASVNQFLTF | CCUGGACAGCAGCU | | |
| | | GGEWRHDKLSDAVS | ACACCCUGAUCCUG | | |
| | | LTGGSSTKTSASQYA | AUCGACGGCAAGAG | | |
| | | LFLEDEWRIFEPLALT | AGUGAACAGCCGGA | | |
| | | TGIRMDDHETYGDH | ACGCCGUGUUCCGG | | |
| | | WSPRAYLVYNATDT | CACAACGACUUCGA | | |
| | | LTVKGGWATAFKAP | CCUGAACUGGAUCC | | |
| | | SLLQLSPDWATNSCR | CCGUGGAUGCCAUC | | |
| | | GGCRIVGSPDLKPETS | GAGAGGAUCGAAGU | | |
| | | ESWELGLYYRGEEGI | UGUGCGGGGACCUA | | |
| | | LEGVEASVTTFRNDV | UGAGCAGCCUGUAC | | |
| | | DNRISISRTPDVNAAP | GGAUCUGAUGCCCU | | |
| | | GYSNFVGFETNSRGQ | CGGCGGCGUGGUCA | | |
| | | RVPVFRYYNVNKARI | ACAUCAUCACCAAG | | |
| | | QGVETELKVPFNEA | AAGAUCGGCCAGAA | | |
| | | WKLSLNYTYNDGRD | AUGGCACGGCAGCG | | |
| | | VSNGGNKPLSDLPFH | UGACCGUGGAUAGC | | |
| | | TANGTLDWKPAQLE | ACCAUCCAAGAGCA | | |
| | | DWSFYVSGNYTGRK | CAGAGACAGAGGCG | | |
| | | RADSATAKTPGGYV | ACACCUACAACGGC | | |
| | | VWDTGAAWQATKN | CAGUUCUUCACAAG | | |
| | | VKLRAGVLNVGDKD | CGGCCCUCUGAUUG | | |
| | | LKRDDYGYTEDGRR | AUGGCGUGCUGGGC | | |
| | | YFMAVDYRFHHHHH | AUGAAGGCCUACGG | | |
| | | H | AUCUCUGGCCAAGA | | |
| | | | GAGAGAAGGACGAG | | |
| | | | CAGCAGAGCAGCGC | | |
| | | | CACAACAGCCACAG | | |
| | | | GCGAGACACCUAGA | | |
| | | | AUCGAGGGCUUCAC | | |
| | | | CAGCAGAGAUGGCA | | |
| | | | ACGUGGAAUUCGCC | | |
| | | | UGGACACCCAACGA | | |
| | | | GAACCACGAUGUGA | | |
| | | | CAGCCGGCUACGGC | | |
| | | | UUCGACAGACAGGA | | |
| | | | CAGAGAUAGCGACA | | |
| | | | GCCUGGACAAGAAC | | |
| | | | CGGCUGGAAAGACA | | |
| | | | GAACUACGCCCUGA | | |
| | | | GCCACAACGGCAGA | | |
| | | | UGGGACCUGGGAAA | | |
| | | | CAGCGAGCUGAAGU | | |
| | | | UCUACGGCGAGAAG | | |
| | | | GUCGAGAACAAGAA | | |
| | | | CCCCGGCAACAGCA | | |
| | | | GCCCUAUCACCAGC | | |
| | | | GAGAGCAACAGCAU | | |
| | | | CGAUGGCAAAUACG | | |
| | | | UGCUGCCCCUGGCC | | |
| | | | UCCGUGAAUCAGUU | | |
| | | | CCUGACAUUUGGCG | | |
| | | | GAGAGUGGCGGCAC | | |
| | | | GACAAGCUGUCUGA | | |
| | | | UGCCGUUUCUCUGA | | |
| | | | CCGGCGGCAGCAGC | | |
| | | | ACAAAGACAAGCGC | | |
| | | | CUCUCAGUACGCCC | | |
| | | | UGUUCCUGGAAGAU | | |
| | | | GAGUGGCGGAUCUU | | |
| | | | CGAGCCCCUGGCUC | | |
| | | | UGACAACAGGCAUC | | |
| | | | AGAAUGGACGACCA | | |
| | | | CGAGACAUACGGCG | | |
| | | | ACCACUGGUCCCCA | | |
| | | | AGAGCCUACCUGGU | | |
| | | | GUACAACGCCACCG | | |
| | | | ACACACUGACCGUG | | |
| | | | AAAGGCGGAUGGGC | | |
| | | | CACAGCCUUUAAGG | | |
| | | | CUCCUAGUCUGCUG | | |
| | | | CAGCUGAGCCCCGA | | |
| | | | UUGGGCCACCAAUU | | |
| | | | CUUGUAGAGGCGGC | | |
| | | | UGCAGAAUCGUGGG | | |
| | | | CAGCCCUGAUCUGA | | |
| | | | AGCCCGAGACAUCU | | |
| | | | GAGUCUUGGGAGCU | | |
| | | | GGGACUGUACUACA | | |
| | | | GGGGCGAAGAGGGA | | |
| | | | AUCCUGGAAGGCGU | | |
| | | | GGAAGCCAGCGUGA | | |
| | | | CAACCUUCAGAAAC | | |
| | | | GACGUGGACAACCG | | |
| | | | GAUCAGCAUCUCCA | | |
| | | | GAACACCCGACGUG | | |
| | | | AACGCCGCUCCUGG | | |
| | | | CUACUCUAAUUUCG | | |
| | | | AACAGCAGAGGCCA | | |
| | | | GAGGGUGCCCGUGU | | |
| | | | UUCGGUACUACAAC | | |
| | | | GUGAACAAGGCCCG | | |
| | | | GAUCCAGGGCGUCG | | |
| | | | AGACAGAGCUGAAG | | |
| | | | GUGCCCUUUAACGA | | |
| | | | AGCCUGGAAGCUGA | | |
| | | | GCCUGAACUACACA | | |
| | | | UACAACGACGGCCG | | |
| | | | GGACGUGUCCAACG | | |
| | | | GCGGAAACAAACCU | | |
| | | | CUGAGCGACCUGCC | | |
| | | | UUUCCACACCGCCA | | |
| | | | AUGGCACCCUGGAU | | |
| | | | UGGAAGCCUGCUCA | | |
| | | | GCUGGAAGAUUGGA | | |
| | | | GCUUCUACGUGUCC | | |
| | | | GGCAACUACACCGG | | |
| | | | CAGAAAGAGAGCCG | | |
| | | | AUAGCGCCACCGCU | | |
| | | | AAGACACCAGGCGG | | |
| | | | AUACGUCGUGUGGG | | |
| | | | AUACAGGUGCUGCU | | |
| | | | UGGCAGGCCACCAA | | |
| | | | GAACGUGAAACUGA | | |
| | | | GAGCCGGCGUGCUG | | |
| | | | AACGUGGGCGACAA | | |
| | | | AGACCUGAAGAGGG | | |
| | | | ACGACUACGGCUAC | | |
| | | | ACCGAGGACGGCAG | | |
| | | | ACGGUACUUCAUGG | | |
| | | | CCGUGGACUACCGG | | |
| | | | UUCCACCACCACCA | | |
| | | | UCACCAU | | |
| SEQ ID NO: | | 81 | 82 | 3 | 4 |

SEQ ID NO: 200 consists of from 5′ end to 3′ end, 5′ UTR SEQ
ID NO: 3, mRNA ORF SEQ ID NO: 82, and 3′ UTR SEQ ID NO: 4.

| | St_CirA_n | MGLQTTKWPSHGAF | AUGGGCCUGCAGAC | GGGAAA | UGAUAA |
|---|---|---|---|---|---|
| | FLRT2 | FLKSWLIISLGLYSQV | CACAAAGUGGCCUU | UAAGAG | UAGGCU |
| | | SKLLAATDDGETMV | CUCACGGCGCAUUC | AGAAAA | GGAGCC |
| | | VTASAIEQNLKDAPA | UUUCUGAAGUCCUG | GAAGAG | UCGGUG |
| | | SISVITQQDLQRRPVQ | GCUGAUCAUCUCCC | UAAGAA | GCCAUG |
| | | NLKDVLKEVPGVQL | UGGGCCUGUACAGC | GAAAUA | CUUCUU |
| | | TNEGDNRKGVSIRGL | CAGGUGUCCAAACU | UAAGAG | GCCCCU |
| | | DSSYTLILIDGKRVNS | GCUGGCCGCCACCG | CCACC | UGGGCC |
| | | RNAVFRHNDFDLNW | AUGAUGGCGAGACA | | UCCCCC |
| | | IPVDAIERIEVVRGPM | AUGGUGGUUACAGC | | CAGCCC |
| | | SSLYGSDALGGVVNII | CAGCGCCAUCGAGC | | CUCCUC |
| | | TKKIGQKWHGSVTV | AGAACCUGAAAGAU | | CCCUUC |
| | | DSTIQEHRDRGDTYN | GCCCCUGCCAGCAU | | CUGCAC |
| | | GQFFTSGPLIDGVLG | CAGCGUGAUCACCC | | CCGUAC |
| | | MKAYGSLAKREKDE | AGCAGGAUCUGCAG | | CCCCGU |
| | | QQSSATTATGETPRIE | AGAAGGCCCGUGCA | | GGUCUU |
| | | GFTSRDGNVEFAWTP | GAAUCUGAAGGACG | | UGAAUA |
| | | NENHDVTAGYGFDR | UGCUGAAAGAGGUG | | AAGUCU |
| | | QDRDSDSLDKNRLER | CCCGGCGUCCAGCU | | GAGUGG |
| | | QNYALSHNGRWDLG | GACAAACGAGGGCG | | GCGGC |
| | | NSELKFYGEKVENKN | AUAAUAGAAAGGGC | | |
| | | PGNSSPITSESNSIDG | GUGUCCAUCAGAGG | | |
| | | KYVLPLASVNQFLTF | CCUGGACAGCAGCU | | |
| | | GGEWRHDKLSDAVS | ACACCCUGAUCCUG | | |
| | | LTGGSSTKTSASQYA | AUCGACGGCAAGAG | | |
| | | LFLEDEWRIFEPLALT | AGUGAACAGCCGGA | | |
| | | TGIRMDDHETYGDH | ACGCCGUGUUCCGG | | |
| | | WSPRAYLVYNATDT | CACAACGACUUCGA | | |
| | | LTVKGGWATAFKAP | CCUGAACUGGAUCC | | |
| | | SLLQLSPDWATNSCR | CCGUGGAUGCCAUC | | |
| | | GGCRIVGSPDLKPETS | GAGAGGAUCGAAGU | | |
| | | ESWELGLYYRGEEGI | UGUGCGGGGACCUA | | |
| | | LEGVEASVTTFRNDV | UGAGCAGCCUGUAC | | |
| | | DNRISISRTPDVNAAP | GGAUCUGAUGCCCU | | |
| | | GYSNFVGFETNSRGQ | CGGCGGCGUGGUCA | | |
| | | RVPVFRYYNVNKARI | ACAUCAUCACCAAG | | |
| | | QGVETELKVPFNEA | AAGAUCGGCCAGAA | | |
| | | WKLSLNYTYNDGRD | AUGGCACGGCAGCG | | |
| | | VSNGGNKPLSDLPFH | UGACCGUGGAUAGC | | |
| | | TANGTLDWKPAQLE | ACCAUCCAAGAGCA | | |
| | | DWSFYVSGNYTGRK | CAGAGACAGAGGCG | | |
| | | RADSATAKTPGGYV | ACACCUACAACGGC | | |
| | | VWDTGAAWQATKN | CAGUUCUUCACAAG | | |
| | | VKLRAGVLNVGDKD | CGGCCCUCUGAUUG | | |
| | | LKRDDYGYTEDGRR | AUGGCGUGCUGGGC | | |
| | | YFMAVDYRF | AUGAAGGCCUACGG | | |
| | | | AUCUCUGGCCAAGA | | |
| | | | GAGAGAAGGACGAG | | |
| | | | CAGCAGAGCAGCGC | | |
| | | | CACAACAGCCACAG | | |
| | | | GCGAGACACCUAGA | | |
| | | | AUCGAGGGCUUCAC | | |
| | | | CAGCAGAGAUGGCA | | |
| | | | ACGUGGAAUUCGCC | | |
| | | | UGGACACCCAACGA | | |
| | | | GAACCACGAUGUGA | | |
| | | | CAGCCGGCUACGGC | | |
| | | | UUCGACAGACAGGA | | |
| | | | CAGAGAUAGCGACA | | |
| | | | GCCUGGACAAGAAC | | |
| | | | CGGCUGGAAAGACA | | |
| | | | GAACUACGCCCUGA | | |
| | | | GCCACAACGGCAGA | | |
| | | | UGGGACCUGGGAAA | | |
| | | | CAGCGAGCUGAAGU | | |
| | | | UCUACGGCGAGAAG | | |
| | | | GUCGAGAACAAGAA | | |
| | | | CCCCGGCAACAGCA | | |
| | | | GCCCUAUCACCAGC | | |
| | | | GAGAGCAACAGCAU | | |
| | | | CGAUGGCAAAUACG | | |
| | | | UGCUGCCCCUGGCC | | |
| | | | UCCGUGAAUCAGUU | | |
| | | | CCUGACAUUUGGCG | | |
| | | | GAGAGUGGCGGCAC | | |
| | | | GACAAGCUGUCUGA | | |
| | | | UGCCGUUUCUCUGA | | |
| | | | CCGGCGGCAGCAGC | | |
| | | | CUCUCAGUACGCCC | | |
| | | | UGUUCCUGGAAGAU | | |
| | | | GAGUGGCGGAUCUU | | |
| | | | CGAGCCCCUGGCUC | | |
| | | | UGACAACAGGCAUC | | |
| | | | AGAAUGGACGACCA | | |
| | | | CGAGACAUACGGCG | | |
| | | | ACCACUGGUCCCCA | | |
| | | | AGAGCCUACCUGGU | | |
| | | | GUACAACGCCACCG | | |
| | | | ACACACUGACCGUG | | |
| | | | AAAGGCGGAUGGGC | | |
| | | | CACAGCCUUUAAGG | | |
| | | | CUCCUAGUCUGCUG | | |
| | | | CAGCUGAGCCCCGA | | |
| | | | UUGGGCCACCAAUU | | |
| | | | CUUGUAGAGGCGGC | | |
| | | | UGCAGAAUCGUGGG | | |
| | | | CAGCCCUGAUCUGA | | |
| | | | AGCCCGAGACAUCU | | |
| | | | GAGUCUUGGGAGCU | | |
| | | | GGGACUGUACUACA | | |
| | | | GGGGCGAAGAGGGA | | |
| | | | AUCCUGGAAGGCGU | | |
| | | | GGAAGCCAGCGUGA | | |
| | | | CAACCUUCAGAAAC | | |
| | | | GACGUGGACAACCG | | |
| | | | GAUCAGCAUCUCCA | | |
| | | | GAACACCCGACGUG | | |
| | | | AACGCCGCUCCUGG | | |
| | | | CUACUCUAAUUUCG | | |
| | | | UGGGCUUCGAGACA | | |
| | | | AACAGCAGAGGCCA | | |
| | | | GAGGGUGCCCGUGU | | |
| | | | UUCGGUACUACAAC | | |
| | | | GUGAACAAGGCCCG | | |
| | | | GAUCCAGGGCGUCG | | |
| | | | AGACAGAGCUGAAG | | |
| | | | GUGCCCUUUAACGA | | |
| | | | AGCCUGGAAGCUGA | | |
| | | | GCCUGAACUACACA | | |
| | | | UACAACGACGGCCG | | |
| | | | GGACGUGUCCAACG | | |
| | | | GCGGAAACAAACCU | | |
| | | | CUGAGCGACCUGCC | | |
| | | | UUUCCACACCGCCA | | |
| | | | AUGGCACCCUGGAU | | |
| | | | UGGAAGCCUGCUCA | | |
| | | | GCUGGAAGAUUGGA | | |
| | | | GCUUCUACGUGUCC | | |
| | | | GGCAACUACACCGG | | |
| | | | CAGAAAGAGAGCCG | | |
| | | | AUAGCGCCACCGCU | | |
| | | | AAGACACCAGGCGG | | |
| | | | AUACGUCGUGUGGG | | |
| | | | AUACAGGUGCUGCU | | |
| | | | UGGCAGGCCACCAA | | |
| | | | GAACGUGAAACUGA | | |
| | | | GAGCCGGCGUGCUG | | |
| | | | AACGUGGGCGACAA | | |
| | | | AGACCUGAAGAGGG | | |
| | | | ACGACUACGGCUAC | | |
| | | | ACCGAGGACGGCAG | | |
| | | | ACGGUACUUCAUGG | | |
| | | | CCGUGGACUACCGG | | |
| | | | UUC | | |
| SEQ ID NO: | | 83 | 84 | 3 | 4 |

SEQ ID NO: 201 consists of from 5′ end to 3′ end, 5′ UTR SEQ
ID NO: 3, mRNA ORF SEQ ID NO: 84, and 3′ UTR SEQ ID NO: 4.

| | St_cirA_N | MGLQTTKWPSHGAF | AUGGGCCUGCAGAC | GGGAAA | UGAUAA |
|---|---|---|---|---|---|
| | GM_nFLR | FLKSWLIISLGLYSQV | CACAAAGUGGCCUU | UAAGAG | UAGGCU |
| | T2_cHis | SKLLAATDDGETMV | CUCACGGCGCAUUC | AGAAAA | GGAGCC |
| | | VTASAIEQNLKDAPA | UUUCUGAAGUCCUG | GAAGAG | UCGGUG |
| | | SISVITQQDLQRRPVQ | GCUGAUCAUCUCCC | UAAGAA | GCCAUG |
| | | NLKDVLKEVPGVQL | UGGGCCUGUACAGC | GAAAUA | CUUCUU |
| | | TNEGDNRKGVSIRGL | CAGGUGUCCAAACU | UAAGAG | GCCCCU |
| | | DSSYTLILIDGKRVNS | GCUGGCCGCCACCG | CCACC | UGGGCC |
| | | RNAVFRHNDFDLNW | AUGAUGGCGAGACA | | UCCCCC |
| | | IPVDAIERIEVVRGPM | AUGGUGGUUACAGC | | CAGCCC |
| | | SSLYGSDALGGVVNII | CAGCGCCAUCGAGC | | CUCCUC |
| | | TKKIGQKWHGSVTV | AGAACCUGAAAGAU | | CCCUUC |
| | | DSTIQEHRDRGDTYN | GCCCCUGCCAGCAU | | CUGCAC |
| | | GQFFTSGPLIDGVLG | CAGCGUGAUCACCC | | CCGUAC |
| | | MKAYGSLAKREKDE | AGCAGGAUCUGCAG | | CCCCGU |
| | | QQSSATTATGETPRIE | AGAAGGCCCGUGCA | | GGUCUU |
| | | GFTSRDGNVEFAWTP | GAAUCUGAAGGACG | | UGAAUA |
| | | NENHDVTAGYGFDR | UGCUGAAAGAGGUG | | AAGUCU |
| | | QDRDSDSLDKNRLER | CCCGGCGUCCAGCU | | GAGUGG |
| | | QNYALSHNGRWDLG | GACAAACGAGGGCG | | GCGGC |
| | | NSELKFYGEKVENKN | AUAAUAGAAAGGGC | | |
| | | PGNSSPITSESNSIDG | GUGUCCAUCAGAGG | | |
| | | KYVLPLASVNQFLTF | CCUGGACAGCAGCU | | |
| | | GGEWRHDKLSDAVS | ACACCCUGAUCCUG | | |
| | | LTGGSSTKTSASQYA | AUCGACGGCAAGAG | | |
| | | LFLEDEWRIFEPLALT | AGUGAACAGCCGGA | | |
| | | TGIRMDDHETYGDH | ACGCCGUGUUCCGG | | |
| | | WSPRAYLVYNAADT | CACAACGACUUCGA | | |
| | | LTVKGGWATAFKAP | CCUGAACUGGAUCC | | |
| | | SLLQLSPDWATNSCR | CCGUGGAUGCCAUC | | |
| | | GGCRIVGSPDLKPETS | GAGAGGAUCGAAGU | | |
| | | ESWELGLYYRGEEGI | UGUGCGGGGACCUA | | |
| | | LEGVEASVTTFRNDV | UGAGCAGCCUGUAC | | |
| | | DNRISISRTPDVNAAP | GGAUCUGAUGCCCU | | |
| | | GYSNFVGFETNSRGQ | CGGCGGCGUGGUCA | | |
| | | RVPVFRYYNVNKARI | ACAUCAUCACCAAG | | |
| | | QGVETELKVPFNEA | AAGAUCGGCCAGAA | | |
| | | WKLSLNYAYNDGRD | AUGGCACGGCAGCG | | |
| | | VSNGGNKPLSDLPFH | UGACCGUGGAUAGC | | |
| | | TANGALDWKPAQLE | ACCAUCCAAGAGCA | | |
| | | DWSFYVSGNYAGRK | CAGAGACAGAGGCG | | |
| | | RADSATAKTPGGYV | ACACCUACAACGGC | | |
| | | VWDTGAAWQATKN | CAGUUCUUCACAAG | | |
| | | VKLRAGVLNVGDKD | CGGCCCUCUGAUUG | | |
| | | LKRDDYGYTEDGRR | AUGGCGUGCUGGGC | | |
| | | YFMAVDYRFHHHHH | AUGAAGGCCUACGG | | |
| | | H | AUCUCUGGCCAAGA | | |
| | | | GAGAGAAGGACGAG | | |
| | | | CAGCAGAGCAGCGC | | |
| | | | CACAACAGCCACAG | | |
| | | | GCGAGACACCUAGA | | |
| | | | AUCGAGGGCUUCAC | | |
| | | | CAGCAGAGAUGGCA | | |
| | | | ACGUGGAAUUCGCC | | |
| | | | UGGACACCCAACGA | | |
| | | | GAACCACGAUGUGA | | |
| | | | CAGCCGGCUACGGC | | |
| | | | UUCGACAGACAGGA | | |
| | | | CAGAGAUAGCGACA | | |
| | | | GCCUGGACAAGAAC | | |
| | | | CGGCUGGAAAGACA | | |
| | | | GAACUACGCCCUGA | | |
| | | | GCCACAACGGCAGA | | |
| | | | UGGGACCUGGGAAA | | |
| | | | CAGCGAGCUGAAGU | | |
| | | | UCUACGGCGAGAAG | | |
| | | | GUCGAGAACAAGAA | | |
| | | | CCCCGGCAACAGCA | | |
| | | | GCCCUAUCACCAGC | | |
| | | | GAGAGCAACAGCAU | | |
| | | | CGAUGGCAAAUACG | | |
| | | | UGCUGCCCCUGGCC | | |
| | | | UCCGUGAAUCAGUU | | |
| | | | CCUGACAUUUGGCG | | |
| | | | GAGAGUGGCGGCAC | | |
| | | | GACAAGCUGUCUGA | | |
| | | | UGCCGUUUCUCUGA | | |
| | | | CCGGCGGCAGCAGC | | |
| | | | ACAAAGACAAGCGC | | |
| | | | CUCUCAGUACGCCC | | |
| | | | UGUUCCUGGAAGAU | | |
| | | | GAGUGGCGGAUCUU | | |
| | | | CGAGCCCCUGGCUC | | |
| | | | UGACAACAGGCAUC | | |
| | | | AGAAUGGACGACCA | | |
| | | | CGAGACAUACGGCG | | |
| | | | ACCACUGGUCCCCA | | |
| | | | AGAGCCUACCUGGU | | |
| | | | GUACAACGCCGCCG | | |
| | | | ACACACUGACCGUG | | |
| | | | AAAGGCGGAUGGGC | | |
| | | | CACAGCCUUUAAGG | | |
| | | | CUCCUAGUCUGCUG | | |
| | | | CAGCUGAGCCCCGA | | |
| | | | UUGGGCCACCAAUU | | |
| | | | CUUGUAGAGGCGGC | | |
| | | | UGCAGAAUCGUGGG | | |
| | | | CAGCCCUGAUCUGA | | |
| | | | AGCCCGAGACAUCU | | |
| | | | GAGUCUUGGGAGCU | | |
| | | | GGGACUGUACUACA | | |
| | | | GGGGCGAAGAGGGA | | |
| | | | AUCCUGGAAGGCGU | | |
| | | | GGAAGCCAGCGUGA | | |
| | | | CAACCUUCAGAAAC | | |
| | | | GACGUGGACAACCG | | |
| | | | GAUCAGCAUCUCCA | | |
| | | | GAACACCCGACGUG | | |
| | | | AACGCCGCUCCUGG | | |
| | | | CUACUCUAAUUUCG | | |
| | | | UGGGCUUCGAGACA | | |
| | | | AACAGCAGAGGCCA | | |
| | | | GAGGGUGCCCGUGU | | |
| | | | UUCGGUACUACAAC | | |
| | | | GUGAACAAGGCCCG | | |
| | | | GAUCCAGGGCGUCG | | |
| | | | AGACAGAGCUGAAG | | |
| | | | GUGCCCUUUAACGA | | |
| | | | AGCCUGGAAGCUGA | | |
| | | | GCCUGAACUACGCA | | |
| | | | UACAACGACGGCCG | | |
| | | | GGACGUGUCCAACG | | |
| | | | GCGGAAACAAACCU | | |
| | | | CUGAGCGACCUGCC | | |
| | | | UUUCCACACCGCCA | | |
| | | | AUGGCGCCCUGGAU | | |
| | | | UGGAAGCCUGCUCA | | |
| | | | GCUGGAAGAUUGGA | | |
| | | | GCUUCUACGUGUCC | | |
| | | | GGCAACUACGCCGG | | |
| | | | CAGAAAGAGAGCCG | | |
| | | | AUAGCGCCACCGCU | | |
| | | | AAGACACCAGGCGG | | |
| | | | AUACGUCGUGUGGG | | |
| | | | AUACAGGUGCUGCU | | |
| | | | UGGCAGGCCACCAA | | |
| | | | GAACGUGAAACUGA | | |
| | | | GAGCCGGCGUGCUG | | |
| | | | AACGUGGGCGACAA | | |
| | | | AGACCUGAAGAGGG | | |
| | | | ACGACUACGGCUAC | | |
| | | | ACCGAGGACGGCAG | | |
| | | | ACGGUACUUCAUGG | | |
| | | | CCGUGGACUACCGG | | |
| | | | UUCCACCACCACCA | | |
| | | | UCACCAU | | |
| SEQ ID NO: | | 85 | 86 | 3 | 4 |

SEQ ID NO: 202 consists of from 5′ end to 3′ end, 5′ UTR SEQ
ID NO: 3, mRNA ORF SEQ ID NO: 86, and 3′ UTR SEQ ID NO: 4.

| | St_cirA_N | MGLQTTKWPSHGAF | AUGGGCCUGCAGAC | GGGAAA | UGAUAA |
|---|---|---|---|---|---|
| | GM_nFLR | FLKSWLIISLGLYSQV | CACAAAGUGGCCUU | UAAGAG | UAGGCU |
| | T2 | SKLLAATDDGETMV | CUCACGGCGCAUUC | AGAAAA | GGAGCC |
| | | VTASAIEQNLKDAPA | UUUCUGAAGUCCUG | GAAGAG | UCGGUG |
| | | SISVITQQDLQRRPVQ | GCUGAUCAUCUCCC | UAAGAA | GCCAUG |
| | | NLKDVLKEVPGVQL | UGGGCCUGUACAGC | GAAAUA | CUUCUU |
| | | TNEGDNRKGVSIRGL | CAGGUGUCCAAACU | UAAGAG | GCCCCU |
| | | DSSYTLILIDGKRVNS | GCUGGCCGCCACCG | CCACC | UGGGCC |
| | | RNAVFRHNDFDLNW | AUGAUGGCGAGACA | | UCCCCC |
| | | IPVDAIERIEVVRGPM | AUGGUGGUUACAGC | | CAGCCC |
| | | SSLYGSDALGGVVNII | CAGCGCCAUCGAGC | | CUCCUC |
| | | TKKIGQKWHGSVTV | AGAACCUGAAAGAU | | CCCUUC |
| | | DSTIQEHRDRGDTYN | GCCCCUGCCAGCAU | | CUGCAC |
| | | GQFFTSGPLIDGVLG | CAGCGUGAUCACCC | | CCGUAC |
| | | MKAYGSLAKREKDE | AGCAGGAUCUGCAG | | CCCCGU |
| | | QQSSATTATGETPRIE | AGAAGGCCCGUGCA | | GGUCUU |
| | | GFTSRDGNVEFAWTP | GAAUCUGAAGGACG | | UGAAUA |
| | | NENHDVTAGYGFDR | UGCUGAAAGAGGUG | | AAGUCU |
| | | QDRDSDSLDKNRLER | CCCGGCGUCCAGCU | | GAGUGG |
| | | QNYALSHNGRWDLG | GACAAACGAGGGCG | | GCGGC |
| | | NSELKFYGEKVENKN | AUAAUAGAAAGGGC | | |
| | | PGNSSPITSESNSIDG | GUGUCCAUCAGAGG | | |
| | | KYVLPLASVNQFLTF | CCUGGACAGCAGCU | | |
| | | GGEWRHDKLSDAVS | ACACCCUGAUCCUG | | |
| | | LTGGSSTKTSASQYA | AUCGACGGCAAGAG | | |
| | | LFLEDEWRIFEPLALT | AGUGAACAGCCGGA | | |
| | | TGIRMDDHETYGDH | ACGCCGUGUUCCGG | | |
| | | WSPRAYLVYNAADT | CACAACGACUUCGA | | |
| | | LTVKGGWATAFKAP | CCUGAACUGGAUCC | | |
| | | SLLQLSPDWATNSCR | CCGUGGAUGCCAUC | | |
| | | GGCRIVGSPDLKPETS | GAGAGGAUCGAAGU | | |
| | | ESWELGLYYRGEEGI | UGUGCGGGGACCUA | | |
| | | LEGVEASVTTFRNDV | UGAGCAGCCUGUAC | | |
| | | DNRISISRTPDVNAAP | GGAUCUGAUGCCCU | | |
| | | GYSNFVGFETNSRGQ | CGGCGGCGUGGUCA | | |
| | | RVPVFRYYNVNKARI | ACAUCAUCACCAAG | | |
| | | QGVETELKVPFNEA | AAGAUCGGCCAGAA | | |
| | | WKLSLNYAYNDGRD | AUGGCACGGCAGCG | | |
| | | VSNGGNKPLSDLPFH | UGACCGUGGAUAGC | | |
| | | TANGALDWKPAQLE | ACCAUCCAAGAGCA | | |
| | | DWSFYVSGNYAGRK | CAGAGACAGAGGCG | | |
| | | RADSATAKTPGGYV | ACACCUACAACGGC | | |
| | | VWDTGAAWQATKN | CAGUUCUUCACAAG | | |
| | | VKLRAGVLNVGDKD | CGGCCCUCUGAUUG | | |
| | | LKRDDYGYTEDGRR | AUGGCGUGCUGGGC | | |
| | | YFMAVDYRF | AUGAAGGCCUACGG | | |
| | | | AUCUCUGGCCAAGA | | |
| | | | GAGAGAAGGACGAG | | |
| | | | CAGCAGAGCAGCGC | | |
| | | | CACAACAGCCACAG | | |
| | | | GCGAGACACCUAGA | | |
| | | | AUCGAGGGCUUCAC | | |
| | | | CAGCAGAGAUGGCA | | |
| | | | ACGUGGAAUUCGCC | | |
| | | | UGGACACCCAACGA | | |
| | | | GAACCACGAUGUGA | | |
| | | | CAGCCGGCUACGGC | | |
| | | | UUCGACAGACAGGA | | |
| | | | CAGAGAUAGCGACA | | |
| | | | GCCUGGACAAGAAC | | |
| | | | CGGCUGGAAAGACA | | |
| | | | GAACUACGCCCUGA | | |
| | | | GCCACAACGGCAGA | | |
| | | | UGGGACCUGGGAAA | | |
| | | | CAGCGAGCUGAAGU | | |
| | | | UCUACGGCGAGAAG | | |
| | | | GUCGAGAACAAGAA | | |
| | | | CCCCGGCAACAGCA | | |
| | | | GCCCUAUCACCAGC | | |
| | | | GAGAGCAACAGCAU | | |
| | | | CGAUGGCAAAUACG | | |
| | | | UGCUGCCCCUGGCC | | |
| | | | UCCGUGAAUCAGUU | | |
| | | | CCUGACAUUUGGCG | | |
| | | | GAGAGUGGCGGCAC | | |
| | | | GACAAGCUGUCUGA | | |
| | | | UGCCGUUUCUCUGA | | |
| | | | CCGGCGGCAGCAGC | | |
| | | | ACAAAGACAAGCGC | | |
| | | | CUCUCAGUACGCCC | | |
| | | | UGUUCCUGGAAGAU | | |
| | | | GAGUGGCGGAUCUU | | |
| | | | CGAGCCCCUGGCUC | | |
| | | | UGACAACAGGCAUC | | |
| | | | AGAAUGGACGACCA | | |
| | | | CGAGACAUACGGCG | | |
| | | | ACCACUGGUCCCCA | | |
| | | | AGAGCCUACCUGGU | | |
| | | | GUACAACGCCGCCG | | |
| | | | ACACACUGACCGUG | | |
| | | | AAAGGCGGAUGGGC | | |
| | | | CACAGCCUUUAAGG | | |
| | | | CUCCUAGUCUGCUG | | |
| | | | CAGCUGAGCCCCGA | | |
| | | | UUGGGCCACCAAUU | | |
| | | | CUUGUAGAGGCGGC | | |
| | | | UGCAGAAUCGUGGG | | |
| | | | CAGCCCUGAUCUGA | | |
| | | | AGCCCGAGACAUCU | | |
| | | | GAGUCUUGGGAGCU | | |
| | | | GGGACUGUACUACA | | |
| | | | GGGGCGAAGAGGGA | | |
| | | | AUCCUGGAAGGCGU | | |
| | | | GGAAGCCAGCGUGA | | |
| | | | CAACCUUCAGAAAC | | |
| | | | GACGUGGACAACCG | | |
| | | | GAUCAGCAUCUCCA | | |
| | | | GAACACCCGACGUG | | |
| | | | AACGCCGCUCCUGG | | |
| | | | CUACUCUAAUUUCG | | |
| | | | UGGGCUUCGAGACA | | |
| | | | AACAGCAGAGGCCA | | |
| | | | GAGGGUGCCCGUGU | | |
| | | | UUCGGUACUACAAC | | |
| | | | GUGAACAAGGCCCG | | |
| | | | GAUCCAGGGCGUCG | | |
| | | | AGACAGAGCUGAAG | | |
| | | | GUGCCCUUUAACGA | | |
| | | | AGCCUGGAAGCUGA | | |
| | | | GCCUGAACUACGCA | | |
| | | | UACAACGACGGCCG | | |
| | | | GGACGUGUCCAACG | | |
| | | | GCGGAAACAAACCU | | |
| | | | CUGAGCGACCUGCC | | |
| | | | UUUCCACACCGCCA | | |
| | | | AUGGCGCCCUGGAU | | |
| | | | UGGAAGCCUGCUCA | | |
| | | | GCUGGAAGAUUGGA | | |
| | | | GCUUCUACGUGUCC | | |
| | | | GGCAACUACGCCGG | | |
| | | | CAGAAAGAGAGCCG | | |
| | | | AUAGCGCCACCGCU | | |
| | | | AAGACACCAGGCGG | | |
| | | | AUACGUCGUGUGGG | | |
| | | | AUACAGGUGCUGCU | | |
| | | | UGGCAGGCCACCAA | | |
| | | | GAACGUGAAACUGA | | |
| | | | GAGCCGGCGUGCUG | | |
| | | | AACGUGGGCGACAA | | |
| | | | AGACCUGAAGAGGG | | |
| | | | ACGACUACGGCUAC | | |
| | | | ACCGAGGACGGCAG | | |
| | | | ACGGUACUUCAUGG | | |
| | | | CCGUGGACUACCGG | | |
| | | | UUC | | |
| SEQ ID NO: | | 87 | 88 | 3 | 4 |

SEQ ID NO: 203 consists of from 5′ end to 3′ end, 5′ UTR SEQ
ID NO: 3, mRNA ORF SEQ ID NO: 88, and 3′ UTR SEQ ID NO: 4.

| | St_ViMim | MDSKGSSQKGSRLLL | AUGGAUAGCAAGGG | GGGAAA | UGAUAA |
|---|---|---|---|---|---|
| | o_Lumazin | LLVVSNLLLPQGVVG | CAGCAGCCAGAAGG | UAAGAG | UAGGCU |
| | e_cHis | TSHHDSHGLHRVGSG | GCUCCAGACUGCUG | AGAAAA | GGAGCC |
| | | SAMQIYEGKLTAEGL | CUGCUUCUGGUGGU | GAAGAG | UCGGUG |
| | | RFGIVASRFNHALVD | GUCCAACCUGCUUC | UAAGAA | GCCAUG |
| | | RLVEGAIDAIVRHGG | UGCCUCAAGGCGUU | GAAAUA | CUUCUU |
| | | REEDITLVRVPGSWEI | GUGGGCACGAGCCA | UAAGAG | GCCCCU |
| | | PVAAGELARKENISA | CCACGACAGCCACG | CCACC | UGGGCC |
| | | VIAIGVLIRGATPHFD | GGUUGCACAGGGUG | | UCCCCC |
| | | YIASEVSKGLADLSL | GGAAGCGGCAGCGC | | CAGCCC |
| | | ELRKPITFGVITADTL | CAUGCAGAUCUACG | | CUCCUC |
| | | EQAIERAGTKHGNKG | AGGGAAAGCUGACC | | CCCUUC |
| | | WEAALSAIEMANLFK | GCCGAGGGCCUGAG | | CUGCAC |
| | | SLRGGLVPRGSHHHH | AUUUGGAAUCGUGG | | CCGUAC |
| | | HHSAWSHPQFEK | CCAGCCGGUUCAAU | | CCCCGU |
| | | | CACGCCCUGGUGGA | | GGUCUU |
| | | | UAGACUGGUGGAAG | | UGAAUA |
| | | | GCGCCAUCGAUGCC | | AAGUCU |
| | | | AUUGUCAGACACGG | | GAGUGG |
| | | | CGGCAGAGAAGAGG | | GCGGC |
| | | | ACAUCACCCUCGUU | | |
| | | | AGAGUGCCCGGCUC | | |
| | | | UUGGGAGAUUCCUG | | |
| | | | UGGCUGCUGGCGAG | | |
| | | | CUGGCCCGGAAAGA | | |
| | | | GAAUAUCUCUGCCG | | |
| | | | UUAUCGCCAUCGGC | | |
| | | | GUGCUGAUCAGAGG | | |
| | | | CGCCACACCUCACU | | |
| | | | UCGACUAUAUCGCC | | |
| | | | AGCGAGGUGUCCAA | | |
| | | | AGGCCUGGCCGAUC | | |
| | | | UGAGCCUGGAACUG | | |
| | | | AGAAAGCCCAUCAC | | |
| | | | CUUCGGCGUGAUCA | | |
| | | | CCGCUGACACACUG | | |
| | | | GAACAGGCCAUUGA | | |
| | | | GAGAGCCGGCACCA | | |
| | | | AGCACGGCAACAAA | | |
| | | | GGAUGGGAAGCCGC | | |
| | | | UCUGUCCGCCAUCG | | |
| | | | AGAUGGCCAACCUG | | |
| | | | UUCAAGAGCCUGAG | | |
| | | | AGGCGGCCUGGUGC | | |
| | | | CUAGAGGAUCUCAC | | |
| | | | CACCACCAUCACCA | | |
| | | | CAGCGCUUGGAGCC | | |
| | | | AUCCUCAGUUCGAG | | |
| | | | AAG | | |
| SEQ ID NO: | | 89 | 90 | 3 | 4 |

SEQ ID NO: 204 consists of from 5′ end to 3′ end, 5′ UTR SEQ
ID NO: 3, mRNA ORF SEQ ID NO: 90, and 3′ UTR SEQ ID NO: 4.

| | St_FepA_n | METPAQLLFLLLLWL | AUGGAGACACCUGC | GGGAAA | UGAUAA |
|---|---|---|---|---|---|
| | IgK_cHis | PDTTGEEKTDSAALT | CCAGCUGCUGUUCC | UAAGAG | UAGGCU |
| | | NEDTIVVTAAQQNLQ | UGCUGCUGCUGUGG | AGAAAA | GGAGCC |
| | | APGVSTITADEIRKNP | CUGCCUGACACCAC | GAAGAG | UCGGUG |
| | | PARDVSEIIRTMPGV | CGGGGAGGAGAAGA | UAAGAA | GCCAUG |
| | | NLTGNSTSGQRGNNR | CCGACAGCGCAGCC | GAAAUA | CUUCUU |
| | | QIDIRGMGPENTLILI | CUGACCAACGAGGA | UAAGAG | GCCCCU |
| | | DGKPVTSRNSVRLG | CACCAUCGUGGUGA | CCACC | UGGGCC |
| | | WRGERDTRGDTAWV | CCGCCGCUCAGCAG | | UCCCCC |
| | | PPEMIERIEVLRGPAA | AACCUGCAGGCGCC | | CAGCCC |
| | | ARYGNGAAGGVVNII | CGGCGUGAGCACCA | | CUCCUC |
| | | TKKGGSEWHGSWNT | UCACCGCCGACGAG | | CCCUUC |
| | | YFNAPEHKDEGATK | AUCAGAAAGAACCC | | CUGCAC |
| | | RTNFSLNGPLGGDFS | UCCUGCCAGAGACG | | CCGUAC |
| | | FRLYGNLDKTQADA | UGAGCGAGAUCAUC | | CCCCGU |
| | | RNINQGHQSERTGSY | AGAACCAUGCCUGG | | GGUCUU |
| | | ADTLPAGREGVINKD | CGUGAACCUGACCG | | UGAAUA |
| | | INGVVRWDFAPLQSL | GCAACAGCACCAGC | | AAGUCU |
| | | ELEAGYSRQGNLYA | GGCCAGAGAGGCAA | | GAGUGG |
| | | GDTQNTNTNQLVKD | CAACAGACAGAUCG | | GCGGC |
| | | NYGKETNRLYRQNY | ACAUCAGAGGCAUG | | |
| | | SLTWNGGWDNGVTT | GGCCCUGAGAACAC | | |
| | | SNWVQYEHTRNSRM | CCUGAUCCUGAUCG | | |
| | | PEGLAGGTEGIFDPK | ACGGCAAGCCCGUG | | |
| | | ASQKYADADLNDVT | ACCAGCAGGAAUAG | | |
| | | LHSEVSLPFDLLVNQ | CGUGAGACUGGGCU | | |
| | | NLTLGTEWAQQRMK | GGAGAGGGGAGCGC | | |
| | | DQLSNSQTFMGGNIP | GACACAAGGGGCGA | | |
| | | GYSSTNRSPYSKAEIF | UACCGCCUGGGUGC | | |
| | | SLFAENNMELTDSTM | CUCCUGAGAUGAUC | | |
| | | LTPGIRFDHHSIVGDN | GAGAGAAUCGAGGU | | |
| | | WSPSLNLSQGLGDDF | GCUGAGAGGCCCUG | | |
| | | TLKMGIARAYKAPSL | CCGCCGCCAGAUAC | | |
| | | YQTNPNYILYSKGQG | GGGAACGGAGCCGC | | |
| | | CYATGAGTGIGCYM | CGGCGGCGUGGUGA | | |
| | | MGNDDLKAETSINKE | ACAUCAUCACCAAG | | |
| | | IGLEFKRDGWLAGVT | AAGGGCGGCAGCGA | | |
| | | WFRNDYRNKIEAGT | GUGGCACGGCAGCU | | |
| | | VPLQRINNGKTDVYQ | GGAACACCUACUUC | | |
| | | WENVPKAVVEGLEG | AACGCCCCUGAGCA | | |
| | | TLNVPVSDTVNWTN | CAAGGACGAGGGCG | | |
| | | NVTYMLQSKNKETG | CCACCAAGAGAACC | | |
| | | ERLSIIPQYTLNSTLS | AACUUCAGCCUGAA | | |
| | | WQVRQDVSLQSTFT | CGGCCCUCUGGGCG | | |
| | | WYGKQEPKKYDYQG | GCGACUUCAGCUUC | | |
| | | NPVTGTDKQAVSPYS | AGACUGUACGGCAA | | |
| | | IVGLSATWDVTKNVS | CCUGGACAAGACCC | | |
| | | LTGGVDNLFDKRLW | AGGCCGACGCCAGA | | |
| | | REGNAQTVRDTQTG | AACAUCAACCAGGG | | |
| | | GRTWYMSINTHFHH | GAACCGGCAGCUAC | | |
| | | HHHH | GCCGACACCCUGCC | | |
| | | | UGCCGGCAGAGAGG | | |
| | | | GCGUGAUCAACAAG | | |
| | | | GACAUCAACGGCGU | | |
| | | | GGUCCGCUGGGAUU | | |
| | | | UCGCCCCACUGCAG | | |
| | | | AGCCUGGAGCUGGA | | |
| | | | GGCCGGCUACAGCA | | |
| | | | GACAGGGGAACCUG | | |
| | | | UACGCCGGCGAUAC | | |
| | | | CCAGAACACCAACA | | |
| | | | CCAACCAGCUGGUG | | |
| | | | AAGGACAACUACGG | | |
| | | | CAAGGAGACAAAUA | | |
| | | | GACUGUACCGCCAG | | |
| | | | AACUACAGCCUGAC | | |
| | | | CUGGAACGGCGGCU | | |
| | | | GGGACAACGGGGUG | | |
| | | | ACCACCAGCAACUG | | |
| | | | GGUGCAGUACGAGC | | |
| | | | ACACCAGAAACAGC | | |
| | | | AGAAUGCCCGAGGG | | |
| | | | GCUGGCCGGUGGCA | | |
| | | | CUGAAGGCAUCUUC | | |
| | | | GACCCUAAAGCCAG | | |
| | | | CCAGAAAUACGCUG | | |
| | | | ACGCCGAUCUGAAC | | |
| | | | GACGUGACCCUGCA | | |
| | | | CAGCGAGGUGAGCC | | |
| | | | UGCCUUUCGACCUG | | |
| | | | CUCGUCAACCAGAA | | |
| | | | CCUGACCCUGGGCA | | |
| | | | CCGAGUGGGCCCAG | | |
| | | | CAGAGAAUGAAGGA | | |
| | | | CCAGCUGAGCAACA | | |
| | | | GCCAGACCUUCAUG | | |
| | | | GGCGGAAACAUACC | | |
| | | | AGGCUAUAGCAGCA | | |
| | | | CCAACAGAAGCCCU | | |
| | | | UACAGCAAGGCCGA | | |
| | | | GAUCUUCUCCCUGU | | |
| | | | UCGCCGAGAACAAC | | |
| | | | AUGGAGCUGACCGA | | |
| | | | CAGCACCAUGCUGA | | |
| | | | CCCCUGGCAUCAGA | | |
| | | | UUCGACCAUCACAG | | |
| | | | CAUCGUGGGCGACA | | |
| | | | ACUGGAGCCCCAGC | | |
| | | | CUGAACCUGAGCCA | | |
| | | | AGGCCUGGGCGACG | | |
| | | | ACUUCACCCUGAAG | | |
| | | | AUGGGCAUCGCCAG | | |
| | | | AGCCUACAAGGCCC | | |
| | | | CUAGCCUGUACCAG | | |
| | | | ACCAACCCUAACUA | | |
| | | | CAUCCUGUACAGCA | | |
| | | | AGGGCCAGGGCUGU | | |
| | | | UACGCAACCGGCGC | | |
| | | | CGGCACAGGCAUCG | | |
| | | | GCUGCUACAUGAUG | | |
| | | | GGCAACGACGACCU | | |
| | | | GAAGGCCGAAACUA | | |
| | | | GCAUCAACAAGGAG | | |
| | | | AUCGGCCUGGAGUU | | |
| | | | CAAGAGGGACGGCU | | |
| | | | GGCUCGCCGGAGUG | | |
| | | | ACCUGGUUUAGAAA | | |
| | | | CGACUACAGAAACA | | |
| | | | AGAUCGAAGCCGGC | | |
| | | | ACCGUACCUCUUCA | | |
| | | | GCGGAUCAACAACG | | |
| | | | GCAAGACCGACGUG | | |
| | | | UACCAGUGGGAGAA | | |
| | | | CGUGCCUAAGGCCG | | |
| | | | UGGUGGAGGGCCUC | | |
| | | | GAGGGCACCCUCAA | | |
| | | | CGUGCCUGUGAGCG | | |
| | | | ACACCGUGAACUGG | | |
| | | | ACCAACAACGUGAC | | |
| | | | CUACAUGCUGCAGA | | |
| | | | GCAAGAACAAGGAA | | |
| | | | ACCGGCGAGAGACU | | |
| | | | GAGCAUCAUCCCUC | | |
| | | | AGUACACCCUGAAC | | |
| | | | AGCACCCUGAGCUG | | |
| | | | GCAGGUGAGACAGG | | |
| | | | ACGUGUCCCUGCAG | | |
| | | | UCCACCUUUACGUG | | |
| | | | GUACGGCAAGCAGG | | |
| | | | AGCCUAAGAAGUAC | | |
| | | | GACUACCAGGGCAA | | |
| | | | CCCUGUGACCGGCA | | |
| | | | CCGACAAGCAGGCC | | |
| | | | GUCAGCCCAUAUUC | | |
| | | | CAUCGUGGGACUCA | | |
| | | | GCGCCACCUGGGAC | | |
| | | | GUGACCAAGAACGU | | |
| | | | GAGCCUGACCGGAG | | |
| | | | GGGUUGACAACCUG | | |
| | | | UUCGACAAGAGACU | | |
| | | | GUGGAGAGAGGGCA | | |
| | | | ACGCCCAGACCGUG | | |
| | | | AGGGACACUCAGAC | | |
| | | | CGGCGCCUACAUGG | | |
| | | | CCGGCGCUGGGGCC | | |
| | | | UACACCUACAACGA | | |
| | | | GCCCGGUCGGACCU | | |
| | | | GGUACAUGUCCAUC | | |
| | | | AACACCCACUUCCA | | |
| | | | CCAUCACCAUCACC | | |
| | | | AC | | |
| SEQ ID NO: | | 91 | 92 | 3 | 4 |

SEQ ID NO: 205 consists of from 5′ end to 3′ end, 5′ UTR SEQ
ID NO: 3, mRNA ORF SEQ ID NO: 92, and 3′ UTR SEQ ID NO: 4.

| | St_FepA_n | METPAQLLFLLLLWL | AUGGAGACACCUGC | GGGAAA | UGAUAA |
|---|---|---|---|---|---|
| | IgK | PDTTGEEKTDSAALT | CCAGCUGCUGUUCC | UAAGAG | UAGGCU |
| | | NEDTIVVTAAQQNLQ | UGCUGCUGCUGUGG | AGAAAA | GGAGCC |
| | | APGVSTITADEIRKNP | CUGCCUGACACCAC | GAAGAG | UCGGUG |
| | | PARDVSEIIRTMPGV | CGGGGAGGAGAAGA | UAAGAA | GCCAUG |
| | | NLTGNSTSGQRGNNR | CCGACAGCGCAGCC | GAAAUA | CUUCUU |
| | | QIDIRGMGPENTLILI | CUGACCAACGAGGA | UAAGAG | GCCCCU |
| | | DGKPVTSRNSVRLG | CACCAUCGUGGUGA | CCACC | UGGGCC |
| | | WRGERDTRGDTAWV | CCGCCGCUCAGCAG | | UCCCCC |
| | | PPEMIERIEVLRGPAA | AACCUGCAGGCGCC | | CAGCCC |
| | | ARYGNGAAGGVVNII | CGGCGUGAGCACCA | | CUCCUC |
| | | TKKGGSEWHGSWNT | UCACCGCCGACGAG | | CCCUUC |
| | | YFNAPEHKDEGATK | AUCAGAAAGAACCC | | CUGCAC |
| | | RTNFSLNGPLGGDFS | UCCUGCCAGAGACG | | CCGUAC |
| | | FRLYGNLDKTQADA | UGAGCGAGAUCAUC | | CCCCGU |
| | | RNINQGHQSERTGSY | AGAACCAUGCCUGG | | GGUCUU |
| | | ADTLPAGREGVINKD | CGUGAACCUGACCG | | UGAAUA |
| | | INGVVRWDFAPLQSL | GCAACAGCACCAGC | | AAGUCU |
| | | ELEAGYSRQGNLYA | GGCCAGAGAGGCAA | | GAGUGG |
| | | GDTQNTNTNQLVKD | CAACAGACAGAUCG | | GCGGC |
| | | NYGKETNRLYRQNY | ACAUCAGAGGCAUG | | |
| | | SLTWNGGWDNGVTT | GGCCCUGAGAACAC | | |
| | | SNWVQYEHTRNSRM | CCUGAUCCUGAUCG | | |
| | | PEGLAGGTEGIFDPK | ACGGCAAGCCCGUG | | |
| | | ASQKYADADLNDVT | ACCAGCAGGAAUAG | | |
| | | LHSEVSLPFDLLVNQ | CGUGAGACUGGGCU | | |
| | | NLTLGTEWAQQRMK | GGAGAGGGGAGCGC | | |
| | | DQLSNSQTFMGGNIP | GACACAAGGGGCGA | | |
| | | GYSSTNRSPYSKAEIF | UACCGCCUGGGUGC | | |
| | | SLFAENNMELTDSTM | CUCCUGAGAUGAUC | | |
| | | LTPGIRFDHHSIVGDN | GAGAGAAUCGAGGU | | |
| | | WSPSLNLSQGLGDDF | GCUGAGAGGCCCUG | | |
| | | TLKMGIARAYKAPSL | CCGCCGCCAGAUAC | | |
| | | YQTNPNYILYSKGQG | GGGAACGGAGCCGC | | |
| | | CYATGAGTGIGCYM | CGGCGGCGUGGUGA | | |
| | | MGNDDLKAETSINKE | ACAUCAUCACCAAG | | |
| | | IGLEFKRDGWLAGVT | AAGGGCGGCAGCGA | | |
| | | WFRNDYRNKIEAGT | GUGGCACGGCAGCU | | |
| | | VPLQRINNGKTDVYQ | GGAACACCUACUUC | | |
| | | WENVPKAVVEGLEG | AACGCCCCUGAGCA | | |
| | | TLNVPVSDTVNWTN | CAAGGACGAGGGCG | | |
| | | NVTYMLQSKNKETG | CCACCAAGAGAACC | | |
| | | ERLSIIPQYTLNSTLS | AACUUCAGCCUGAA | | |
| | | WQVRQDVSLQSTFT | CGGCCCUCUGGGCG | | |
| | | WYGKQEPKKYDYQG | GCGACUUCAGCUUC | | |
| | | NPVTGTDKQAVSPYS | AGACUGUACGGCAA | | |
| | | IVGLSATWDVTKNVS | CCUGGACAAGACCC | | |
| | | LTGGVDNLFDKRLW | AGGCCGACGCCAGA | | |
| | | REGNAQTVRDTQTG | AACAUCAACCAGGG | | |
| | | AYMAGAGAYTYNEP | CCACCAGAGCGAGA | | |
| | | GRTWYMSINTHF | GAACCGGCAGCUAC | | |
| | | | GCCGACACCCUGCC | | |
| | | | UGCCGGCAGAGAGG | | |
| | | | GCGUGAUCAACAAG | | |
| | | | GACAUCAACGGCGU | | |
| | | | GGUCCGCUGGGAUU | | |
| | | | UCGCCCCACUGCAG | | |
| | | | AGCCUGGAGCUGGA | | |
| | | | GGCCGGCUACAGCA | | |
| | | | GACAGGGGAACCUG | | |
| | | | UACGCCGGCGAUAC | | |
| | | | CCAGAACACCAACA | | |
| | | | CCAACCAGCUGGUG | | |
| | | | AAGGACAACUACGG | | |
| | | | CAAGGAGACAAAUA | | |
| | | | GACUGUACCGCCAG | | |
| | | | AACUACAGCCUGAC | | |
| | | | CUGGAACGGCGGCU | | |
| | | | GGGACAACGGGGUG | | |
| | | | ACCACCAGCAACUG | | |
| | | | GGUGCAGUACGAGC | | |
| | | | ACACCAGAAACAGC | | |
| | | | AGAAUGCCCGAGGG | | |
| | | | GCUGGCCGGUGGCA | | |
| | | | CUGAAGGCAUCUUC | | |
| | | | GACCCUAAAGCCAG | | |
| | | | CCAGAAAUACGCUG | | |
| | | | ACGCCGAUCUGAAC | | |
| | | | GACGUGACCCUGCA | | |
| | | | CAGCGAGGUGAGCC | | |
| | | | UGCCUUUCGACCUG | | |
| | | | CUCGUCAACCAGAA | | |
| | | | CCUGACCCUGGGCA | | |
| | | | CCGAGUGGGCCCAG | | |
| | | | CAGAGAAUGAAGGA | | |
| | | | CCAGCUGAGCAACA | | |
| | | | GCCAGACCUUCAUG | | |
| | | | GGCGGAAACAUACC | | |
| | | | AGGCUAUAGCAGCA | | |
| | | | CCAACAGAAGCCCU | | |
| | | | UACAGCAAGGCCGA | | |
| | | | GAUCUUCUCCCUGU | | |
| | | | UCGCCGAGAACAAC | | |
| | | | AUGGAGCUGACCGA | | |
| | | | CAGCACCAUGCUGA | | |
| | | | CCCCUGGCAUCAGA | | |
| | | | UUCGACCAUCACAG | | |
| | | | CAUCGUGGGCGACA | | |
| | | | ACUGGAGCCCCAGC | | |
| | | | CUGAACCUGAGCCA | | |
| | | | AGGCCUGGGCGACG | | |
| | | | ACUUCACCCUGAAG | | |
| | | | AUGGGCAUCGCCAG | | |
| | | | AGCCUACAAGGCCC | | |
| | | | CUAGCCUGUACCAG | | |
| | | | ACCAACCCUAACUA | | |
| | | | CAUCCUGUACAGCA | | |
| | | | AGGGCCAGGGCUGU | | |
| | | | UACGCAACCGGCGC | | |
| | | | CGGCACAGGCAUCG | | |
| | | | GCUGCUACAUGAUG | | |
| | | | GGCAACGACGACCU | | |
| | | | GAAGGCCGAAACUA | | |
| | | | GCAUCAACAAGGAG | | |
| | | | AUCGGCCUGGAGUU | | |
| | | | CAAGAGGGACGGCU | | |
| | | | GGCUCGCCGGAGUG | | |
| | | | ACCUGGUUUAGAAA | | |
| | | | CGACUACAGAAACA | | |
| | | | AGAUCGAAGCCGGC | | |
| | | | ACCGUACCUCUUCA | | |
| | | | GCGGAUCAACAACG | | |
| | | | GCAAGACCGACGUG | | |
| | | | UACCAGUGGGAGAA | | |
| | | | CGUGCCUAAGGCCG | | |
| | | | UGGUGGAGGGCCUC | | |
| | | | GAGGGCACCCUCAA | | |
| | | | CGUGCCUGUGAGCG | | |
| | | | ACACCGUGAACUGG | | |
| | | | ACCAACAACGUGAC | | |
| | | | CUACAUGCUGCAGA | | |
| | | | GCAAGAACAAGGAA | | |
| | | | ACCGGCGAGAGACU | | |
| | | | GAGCAUCAUCCCUC | | |
| | | | AGUACACCCUGAAC | | |
| | | | AGCACCCUGAGCUG | | |
| | | | GCAGGUGAGACAGG | | |
| | | | ACGUGUCCCUGCAG | | |
| | | | UCCACCUUUACGUG | | |
| | | | GUACGGCAAGCAGG | | |
| | | | AGCCUAAGAAGUAC | | |
| | | | GACUACCAGGGCAA | | |
| | | | CCCUGUGACCGGCA | | |
| | | | CCGACAAGCAGGCC | | |
| | | | GUCAGCCCAUAUUC | | |
| | | | CAUCGUGGGACUCA | | |
| | | | GCGCCACCUGGGAC | | |
| | | | GUGACCAAGAACGU | | |
| | | | GAGCCUGACCGGAG | | |
| | | | GGGUUGACAACCUG | | |
| | | | UUCGACAAGAGACU | | |
| | | | GUGGAGAGAGGGCA | | |
| | | | ACGCCCAGACCGUG | | |
| | | | AGGGACACUCAGAC | | |
| | | | CGGCGCCUACAUGG | | |
| | | | CCGGCGCUGGGGCC | | |
| | | | UACACCUACAACGA | | |
| | | | GCCCGGUCGGACCU | | |
| | | | GGUACAUGUCCAUC | | |
| | | | AACACCCACUUC | | |
| SEQ ID NO: | | 93 | 94 | 3 | 4 |

SEQ ID NO: 206 consists of from 5′ end to 3′ end, 5′ UTR SEQ
ID NO: 3, mRNA ORF SEQ ID NO: 94, and 3′ UTR SEQ ID NO: 4.

| | St_FepA_ | METPAQLLFLLLLWL | AUGGAGACACCUGC | GGGAAA | UGAUAA |
|---|---|---|---|---|---|
| | NGM_nIg | PDTTGEEKTDSAALT | CCAGCUGCUGUUCC | UAAGAG | UAGGCU |
| | K_cHis | NEDTIVVTAAQQNLQ | UGCUGCUGCUGUGG | AGAAAA | GGAGCC |
| | | APGVSTITADEIRKNP | CUGCCUGACACCAC | GAAGAG | UCGGUG |
| | | PARDVSEIIRTMPGV | CGGGGAGGAGAAGA | UAAGAA | GCCAUG |
| | | NLAGNSASGQRGNN | CCGACAGCGCAGCC | GAAAUA | CUUCUU |
| | | RQIDIRGMGPENTLIL | CUGACCAACGAGGA | UAAGAG | GCCCCU |
| | | IDGKPVTSRNSVRLG | CACCAUCGUGGUGA | CCACC | UGGGCC |
| | | WRGERDTRGDTAWV | CCGCCGCUCAGCAG | | UCCCCC |
| | | PPEMIERIEVLRGPAA | AACCUGCAGGCGCC | | CAGCCC |
| | | ARYGNGAAGGVVNII | CGGCGUGAGCACCA | | CUCCUC |
| | | TKKGGSEWHGSWNT | UCACCGCCGACGAG | | CCCUUC |
| | | YFNAPEHKDEGATK | AUCAGAAAGAACCC | | CUGCAC |
| | | RTNFALNGPLGGDFS | UCCUGCCAGAGACG | | CCGUAC |
| | | FRLYGNLDKTQADA | UGAGCGAGAUCAUC | | CCCCGU |
| | | RNINQGHQSERTGSY | AGAACCAUGCCUGG | | GGUCUU |
| | | ADTLPAGREGVINKD | CGUGAACCUGGCCG | | UGAAUA |
| | | INGVVRWDFAPLQSL | GCAACAGCGCCAGC | | AAGUCU |
| | | ELEAGYSRQGNLYA | GGCCAGAGAGGCAA | | GAGUGG |
| | | GDTQNTNTNQLVKD | CAACAGACAGAUCG | | GCGGC |
| | | NYGKETNRLYRQNY | ACAUCAGAGGCAUG | | |
| | | ALTWNGGWDNGVT | GGCCCUGAGAACAC | | |
| | | TSNWVQYEHTRNSR | CCUGAUCCUGAUCG | | |
| | | MPEGLAGGTEGIFDP | ACGGCAAGCCCGUG | | |
| | | KASQKYADADLNDV | ACCAGCAGGAAUAG | | |
| | | TLHSEVSLPFDLLVN | CGUGAGACUGGGCU | | |
| | | QNLALGTEWAQQRM | GGAGAGGGGAGCGC | | |
| | | KDQLSNSQTFMGGNI | GACACAAGGGGCGA | | |
| | | PGYSSTNRSPYSKAEI | UACCGCCUGGGUGC | | |
| | | FSLFAENNMELTDST | CUCCUGAGAUGAUC | | |
| | | MLTPGIRFDHHSIVG | GAGAGAAUCGAGGU | | |
| | | DNWSPSLNLSQGLGD | GCUGAGAGGCCCUG | | |
| | | DFTLKMGIARAYKAP | CCGCCGCCAGAUAC | | |
| | | SLYQTNPNYILYSKG | GGGAACGGAGCCGC | | |
| | | QGCYATGAGTGIGC | CGGCGGCGUGGUGA | | |
| | | YMMGNDDLKAETSI | ACAUCAUCACCAAG | | |
| | | NKEIGLEFKRDGWLA | AAGGGCGGCAGCGA | | |
| | | GVTWFRNDYRNKIE | GUGGCACGGCAGCU | | |
| | | AGTVPLQRINNGKTD | GGAACACCUACUUC | | |
| | | VYQWENVPKAVVEG | AACGCCCCUGAGCA | | |
| | | LEGTLNVPVSDTVN | CAAGGACGAGGGCG | | |
| | | WANNVAYMLQSKN | CCACCAAGAGAACC | | |
| | | KETGERLSIIPQYTLN | AACUUCGCACUGAA | | |
| | | SALSWQVRQDVSLQ | CGGCCCUCUGGGCG | | |
| | | STFTWYGKQEPKKY | GCGACUUCAGCUUC | | |
| | | DYQGNPVTGTDKQA | AGACUGUACGGCAA | | |
| | | VSPYSIVGLSATWDV | CCUGGACAAGACCC | | |
| | | TKNVSLTGGVDNLFD | AGGCCGACGCCAGA | | |
| | | KRLWREGNAQTVRD | AACAUCAACCAGGG | | |
| | | TQTGAYMAGAGAYT | CCACCAGAGCGAGA | | |
| | | YNEPGRTWYMSINT | GAACCGGCAGCUAC | | |
| | | HFHHHHHH | GCCGACACCCUGCC | | |
| | | | UGCCGGCAGAGAGG | | |
| | | | GCGUGAUCAACAAG | | |
| | | | GACAUCAACGGCGU | | |
| | | | GGUCCGCUGGGAUU | | |
| | | | UCGCCCCACUGCAG | | |
| | | | AGCCUGGAGCUGGA | | |
| | | | GGCCGGCUACAGCA | | |
| | | | GACAGGGGAACCUG | | |
| | | | UACGCCGGCGAUAC | | |
| | | | CCAGAACACCAACA | | |
| | | | CCAACCAGCUGGUG | | |
| | | | AAGGACAACUACGG | | |
| | | | CAAGGAGACAAAUA | | |
| | | | GACUGUACCGCCAG | | |
| | | | AACUACGCACUGAC | | |
| | | | CUGGAACGGCGGCU | | |
| | | | GGGACAACGGGGUG | | |
| | | | ACCACCAGCAACUG | | |
| | | | GGUGCAGUACGAGC | | |
| | | | ACACCAGAAACAGC | | |
| | | | AGAAUGCCCGAGGG | | |
| | | | GCUGGCCGGUGGCA | | |
| | | | CUGAAGGCAUCUUC | | |
| | | | GACCCUAAAGCCAG | | |
| | | | CCAGAAAUACGCUG | | |
| | | | ACGCCGAUCUGAAC | | |
| | | | GACGUGACCCUGCA | | |
| | | | CAGCGAGGUGAGCC | | |
| | | | UGCCUUUCGACCUG | | |
| | | | CUCGUCAACCAGAA | | |
| | | | CCUGGCCCUGGGCA | | |
| | | | CCGAGUGGGCCCAG | | |
| | | | CAGAGAAUGAAGGA | | |
| | | | CCAGCUGAGCAACA | | |
| | | | GCCAGACCUUCAUG | | |
| | | | GGCGGAAACAUACC | | |
| | | | AGGCUAUAGCAGCA | | |
| | | | CCAACAGAAGCCCU | | |
| | | | UACAGCAAGGCCGA | | |
| | | | GAUCUUCUCCCUGU | | |
| | | | UCGCCGAGAACAAC | | |
| | | | AUGGAGCUGACCGA | | |
| | | | CAGCACCAUGCUGA | | |
| | | | CCCCUGGCAUCAGA | | |
| | | | UUCGACCAUCACAG | | |
| | | | CAUCGUGGGCGACA | | |
| | | | ACUGGAGCCCCAGC | | |
| | | | CUGAACCUGAGCCA | | |
| | | | AGGCCUGGGCGACG | | |
| | | | ACUUCACCCUGAAG | | |
| | | | AUGGGCAUCGCCAG | | |
| | | | AGCCUACAAGGCCC | | |
| | | | CUAGCCUGUACCAG | | |
| | | | ACCAACCCUAACUA | | |
| | | | CAUCCUGUACAGCA | | |
| | | | AGGGCCAGGGCUGU | | |
| | | | UACGCAACCGGCGC | | |
| | | | CGGCACAGGCAUCG | | |
| | | | GCUGCUACAUGAUG | | |
| | | | GGCAACGACGACCU | | |
| | | | GAAGGCCGAAACUA | | |
| | | | GCAUCAACAAGGAG | | |
| | | | AUCGGCCUGGAGUU | | |
| | | | CAAGAGGGACGGCU | | |
| | | | GGCUCGCCGGAGUG | | |
| | | | ACCUGGUUUAGAAA | | |
| | | | CGACUACAGAAACA | | |
| | | | AGAUCGAAGCCGGC | | |
| | | | ACCGUACCUCUUCA | | |
| | | | GCGGAUCAACAACG | | |
| | | | GCAAGACCGACGUG | | |
| | | | UACCAGUGGGAGAA | | |
| | | | CGUGCCUAAGGCCG | | |
| | | | UGGUGGAGGGCCUC | | |
| | | | GAGGGCACCCUCAA | | |
| | | | CGUGCCUGUGAGCG | | |
| | | | ACACCGUGAACUGG | | |
| | | | GCCAACAACGUGGC | | |
| | | | CUACAUGCUGCAGA | | |
| | | | GCAAGAACAAGGAA | | |
| | | | GAGCAUCAUCCCUC | | |
| | | | AGUACACCCUGAAC | | |
| | | | AGCGCCCUGAGCUG | | |
| | | | GCAGGUGAGACAGG | | |
| | | | ACGUGUCCCUGCAG | | |
| | | | UCCACCUUUACGUG | | |
| | | | GUACGGCAAGCAGG | | |
| | | | AGCCUAAGAAGUAC | | |
| | | | GACUACCAGGGCAA | | |
| | | | CCCUGUGACCGGCA | | |
| | | | CCGACAAGCAGGCC | | |
| | | | GUCAGCCCAUAUUC | | |
| | | | CAUCGUGGGACUCA | | |
| | | | GCGCCACCUGGGAC | | |
| | | | GUGACCAAGAACGU | | |
| | | | GAGCCUGACCGGAG | | |
| | | | GGGUUGACAACCUG | | |
| | | | UUCGACAAGAGACU | | |
| | | | GUGGAGAGAGGGCA | | |
| | | | ACGCCCAGACCGUG | | |
| | | | AGGGACACUCAGAC | | |
| | | | CGGCGCCUACAUGG | | |
| | | | CCGGCGCUGGGGCC | | |
| | | | UACACCUACAACGA | | |
| | | | GCCCGGUCGGACCU | | |
| | | | GGUACAUGUCCAUC | | |
| | | | AACACCCACUUCCA | | |
| | | | CCAUCACCAUCACC | | |
| | | | AC | | |
| SEQ ID NO: | | 95 | 96 | 3 | 4 |

SEQ ID NO: 207 consists of from 5′ end to 3′ end, 5′ UTR SEQ
ID NO: 3, mRNA ORF SEQ ID NO: 96, and 3′ UTR SEQ ID NO: 4.

| | St_FepA_ | METPAQLLFLLLLWL | AUGGAGACACCUGC | GGGAAA | UGAUAA |
|---|---|---|---|---|---|
| | NGM_nIg | PDTTGEEKTDSAALT | CCAGCUGCUGUUCC | UAAGAG | UAGGCU |
| | K | NEDTIVVTAAQQNLQ | UGCUGCUGCUGUGG | AGAAAA | GGAGCC |
| | | APGVSTITADEIRKNP | CUGCCUGACACCAC | GAAGAG | UCGGUG |
| | | PARDVSEIIRTMPGV | CGGGGAGGAGAAGA | UAAGAA | GCCAUG |
| | | NLAGNSASGQRGNN | CCGACAGCGCAGCC | GAAAUA | CUUCUU |
| | | RQIDIRGMGPENTLIL | CUGACCAACGAGGA | UAAGAG | GCCCCU |
| | | IDGKPVTSRNSVRLG | CACCAUCGUGGUGA | CCACC | UGGGCC |
| | | WRGERDTRGDTAWV | CCGCCGCUCAGCAG | | UCCCCC |
| | | PPEMIERIEVLRGPAA | AACCUGCAGGCGCC | | CAGCCC |
| | | ARYGNGAAGGVVNII | CGGCGUGAGCACCA | | CUCCUC |
| | | TKKGGSEWHGSWNT | UCACCGCCGACGAG | | CCCUUC |
| | | YFNAPEHKDEGATK | AUCAGAAAGAACCC | | CUGCAC |
| | | RTNFALNGPLGGDFS | UCCUGCCAGAGACG | | CCGUAC |
| | | FRLYGNLDKTQADA | UGAGCGAGAUCAUC | | CCCCGU |
| | | RNINQGHQSERTGSY | AGAACCAUGCCUGG | | GGUCUU |
| | | ADTLPAGREGVINKD | CGUGAACCUGGCCG | | UGAAUA |
| | | INGVVRWDFAPLQSL | GCAACAGCGCCAGC | | AAGUCU |
| | | ELEAGYSRQGNLYA | GGCCAGAGAGGCAA | | GAGUGG |
| | | GDTQNTNTNQLVKD | CAACAGACAGAUCG | | GCGGC |
| | | NYGKETNRLYRQNY | ACAUCAGAGGCAUG | | |
| | | ALTWNGGWDNGVT | GGCCCUGAGAACAC | | |
| | | TSNWVQYEHTRNSR | CCUGAUCCUGAUCG | | |
| | | MPEGLAGGTEGIFDP | ACGGCAAGCCCGUG | | |
| | | KASQKYADADLNDV | ACCAGCAGGAAUAG | | |
| | | TLHSEVSLPFDLLVN | CGUGAGACUGGGCU | | |
| | | QNLALGTEWAQQRM | GGAGAGGGGAGCGC | | |
| | | KDQLSNSQTFMGGNI | GACACAAGGGGCGA | | |
| | | PGYSSTNRSPYSKAEI | UACCGCCUGGGUGC | | |
| | | FSLFAENNMELTDST | CUCCUGAGAUGAUC | | |
| | | MLTPGIRFDHHSIVG | GAGAGAAUCGAGGU | | |
| | | DNWSPSLNLSQGLGD | GCUGAGAGGCCCUG | | |
| | | DFTLKMGIARAYKAP | CCGCCGCCAGAUAC | | |
| | | SLYQTNPNYILYSKG | GGGAACGGAGCCGC | | |
| | | QGCYATGAGTGIGC | CGGCGGCGUGGUGA | | |
| | | YMMGNDDLKAETSI | ACAUCAUCACCAAG | | |
| | | NKEIGLEFKRDGWLA | AAGGGCGGCAGCGA | | |
| | | GVTWFRNDYRNKIE | GUGGCACGGCAGCU | | |
| | | AGTVPLQRINNGKTD | GGAACACCUACUUC | | |
| | | VYQWENVPKAVVEG | AACGCCCCUGAGCA | | |
| | | LEGTLNVPVSDTVN | CAAGGACGAGGGCG | | |
| | | WANNVAYMLQSKN | CCACCAAGAGAACC | | |
| | | KETGERLSIIPQYTLN | AACUUCGCACUGAA | | |
| | | SALSWQVRQDVSLQ | CGGCCCUCUGGGCG | | |
| | | STFTWYGKQEPKKY | GCGACUUCAGCUUC | | |
| | | DYQGNPVTGTDKQA | AGACUGUACGGCAA | | |
| | | VSPYSIVGLSATWDV | CCUGGACAAGACCC | | |
| | | TKNVSLTGGVDNLFD | AGGCCGACGCCAGA | | |
| | | KRLWREGNAQTVRD | AACAUCAACCAGGG | | |
| | | TQTGAYMAGAGAYT | CCACCAGAGCGAGA | | |
| | | YNEPGRTWYMSINT | GAACCGGCAGCUAC | | |
| | | HF | GCCGACACCCUGCC | | |
| | | | UGCCGGCAGAGAGG | | |
| | | | GCGUGAUCAACAAG | | |
| | | | GACAUCAACGGCGU | | |
| | | | GGUCCGCUGGGAUU | | |
| | | | UCGCCCCACUGCAG | | |
| | | | AGCCUGGAGCUGGA | | |
| | | | GGCCGGCUACAGCA | | |
| | | | GACAGGGGAACCUG | | |
| | | | UACGCCGGCGAUAC | | |
| | | | CCAGAACACCAACA | | |
| | | | CCAACCAGCUGGUG | | |
| | | | AAGGACAACUACGG | | |
| | | | CAAGGAGACAAAUA | | |
| | | | GACUGUACCGCCAG | | |
| | | | AACUACGCACUGAC | | |
| | | | CUGGAACGGCGGCU | | |
| | | | GGGACAACGGGGUG | | |
| | | | ACCACCAGCAACUG | | |
| | | | GGUGCAGUACGAGC | | |
| | | | ACACCAGAAACAGC | | |
| | | | AGAAUGCCCGAGGG | | |
| | | | GCUGGCCGGUGGCA | | |
| | | | CUGAAGGCAUCUUC | | |
| | | | GACCCUAAAGCCAG | | |
| | | | CCAGAAAUACGCUG | | |
| | | | ACGCCGAUCUGAAC | | |
| | | | GACGUGACCCUGCA | | |
| | | | CAGCGAGGUGAGCC | | |
| | | | UGCCUUUCGACCUG | | |
| | | | CUCGUCAACCAGAA | | |
| | | | CCUGGCCCUGGGCA | | |
| | | | CCGAGUGGGCCCAG | | |
| | | | CAGAGAAUGAAGGA | | |
| | | | CCAGCUGAGCAACA | | |
| | | | GCCAGACCUUCAUG | | |
| | | | GGCGGAAACAUACC | | |
| | | | AGGCUAUAGCAGCA | | |
| | | | CCAACAGAAGCCCU | | |
| | | | UACAGCAAGGCCGA | | |
| | | | GAUCUUCUCCCUGU | | |
| | | | UCGCCGAGAACAAC | | |
| | | | AUGGAGCUGACCGA | | |
| | | | CAGCACCAUGCUGA | | |
| | | | CCCCUGGCAUCAGA | | |
| | | | UUCGACCAUCACAG | | |
| | | | CAUCGUGGGCGACA | | |
| | | | ACUGGAGCCCCAGC | | |
| | | | CUGAACCUGAGCCA | | |
| | | | AGGCCUGGGCGACG | | |
| | | | ACUUCACCCUGAAG | | |
| | | | AUGGGCAUCGCCAG | | |
| | | | AGCCUACAAGGCCC | | |
| | | | CUAGCCUGUACCAG | | |
| | | | ACCAACCCUAACUA | | |
| | | | CAUCCUGUACAGCA | | |
| | | | AGGGCCAGGGCUGU | | |
| | | | UACGCAACCGGCGC | | |
| | | | CGGCACAGGCAUCG | | |
| | | | GCUGCUACAUGAUG | | |
| | | | GGCAACGACGACCU | | |
| | | | GAAGGCCGAAACUA | | |
| | | | GCAUCAACAAGGAG | | |
| | | | AUCGGCCUGGAGUU | | |
| | | | CAAGAGGGACGGCU | | |
| | | | GGCUCGCCGGAGUG | | |
| | | | ACCUGGUUUAGAAA | | |
| | | | CGACUACAGAAACA | | |
| | | | AGAUCGAAGCCGGC | | |
| | | | ACCGUACCUCUUCA | | |
| | | | GCGGAUCAACAACG | | |
| | | | GCAAGACCGACGUG | | |
| | | | UACCAGUGGGAGAA | | |
| | | | CGUGCCUAAGGCCG | | |
| | | | UGGUGGAGGGCCUC | | |
| | | | GAGGGCACCCUCAA | | |
| | | | CGUGCCUGUGAGCG | | |
| | | | ACACCGUGAACUGG | | |
| | | | GCCAACAACGUGGC | | |
| | | | CUACAUGCUGCAGA | | |
| | | | GCAAGAACAAGGAA | | |
| | | | ACCGGCGAGAGACU | | |
| | | | GAGCAUCAUCCCUC | | |
| | | | AGUACACCCUGAAC | | |
| | | | AGCGCCCUGAGCUG | | |
| | | | GCAGGUGAGACAGG | | |
| | | | ACGUGUCCCUGCAG | | |
| | | | UCCACCUUUACGUG | | |
| | | | GUACGGCAAGCAGG | | |
| | | | AGCCUAAGAAGUAC | | |
| | | | GACUACCAGGGCAA | | |
| | | | CCCUGUGACCGGCA | | |
| | | | GUCAGCCCAUAUUC | | |
| | | | CAUCGUGGGACUCA | | |
| | | | GCGCCACCUGGGAC | | |
| | | | GUGACCAAGAACGU | | |
| | | | GAGCCUGACCGGAG | | |
| | | | GGGUUGACAACCUG | | |
| | | | UUCGACAAGAGACU | | |
| | | | GUGGAGAGAGGGCA | | |
| | | | ACGCCCAGACCGUG | | |
| | | | AGGGACACUCAGAC | | |
| | | | CGGCGCCUACAUGG | | |
| | | | CCGGCGCUGGGGCC | | |
| | | | UACACCUACAACGA | | |
| | | | GCCCGGUCGGACCU | | |
| | | | GGUACAUGUCCAUC | | |
| | | | AACACCCACUUC | | |
| SEQ ID NO: | | 97 | 98 | 3 | 4 |

SEQ ID NO: 208 consists of from 5′ end to 3′ end, 5′ UTR SEQ
ID NO: 3, mRNA ORF SEQ ID NO: 98, and 3′ UTR SEQ ID NO: 4.

| | St_CdtB_n | METPAQLLFLLLLWL | AUGGAGACUCCUGC | GGGAAA | UGAUAA |
|---|---|---|---|---|---|
| | IgK | PDTTGNISDYKVMT | CCAGCUGCUGUUCC | UAAGAG | UAGGCU |
| | | WNLQGSSASTESKW | UGCUGCUGCUGUGG | AGAAAA | GGAGCC |
| | | NVNVRQLLSGTAGV | CUGCCUGACACCAC | GAAGAG | UCGGUG |
| | | DILMVQEAGAVPTSA | CGGCAACAUCAGCG | UAAGAA | GCCAUG |
| | | VPTGRHIQPFGVGIPI | ACUACAAGGUGAUG | GAAAUA | CUUCUU |
| | | DEYTWNLGTTSRQDI | ACCUGGAACCUGCA | UAAGAG | GCCCCU |
| | | RYIYHSAIDVGARRV | GGGAAGCUCCGCCA | CCACC | UGGGCC |
| | | NLAIVSRQRADNVYV | GCACCGAGAGCAAG | | UCCCCC |
| | | LRPTTVASRPVIGIGL | UGGAACGUGAACGU | | CAGCCC |
| | | GNDVFLTAHALASG | GAGACAGCUCCUGA | | CUCCUC |
| | | GPDAAAIVRVTINFFR | GCGGCACCGCCGGC | | CCCUUC |
| | | QPQMRHLSWFLAGD | GUCGACAUCCUGAU | | CUGCAC |
| | | FNRSPDRLENDLMTE | GGUGCAGGAGGCCG | | CCGUAC |
| | | HLERVVAVLAPTEPT | GAGCCGUCCCUACC | | CCCCGU |
| | | QIGGGILDYGVIVDR | AGCGCCGUGCCUAC | | GGUCUU |
| | | APYSQRVEALRNPQL | CGGCAGACACAUCC | | UGAAUA |
| | | ASDHYPVAFLARSC | AGCCUUUCGGCGUG | | AAGUCU |
| | | | GGCAUCCCUAUCGA | | GAGUGG |
| | | | CGAGUACACCUGGA | | GCGGC |
| | | | AUCUCGGCACCACC | | |
| | | | AGCAGACAGGACAU | | |
| | | | CAGAUACAUCUACC | | |
| | | | ACAGCGCCAUCGAC | | |
| | | | GUGGGCGCCAGAAG | | |
| | | | AGUGAACCUGGCCA | | |
| | | | UCGUGAGCAGACAG | | |
| | | | AGAGCCGACAACGU | | |
| | | | GUACGUGCUGAGGC | | |
| | | | CUACCACCGUGGCC | | |
| | | | AGCAGACCUGUGAU | | |
| | | | CGGCAUCGGCCUGG | | |
| | | | GCAACGACGUGUUC | | |
| | | | CUGACCGCCCACGC | | |
| | | | UCUGGCCUCCGGUG | | |
| | | | GCCCCGACGCUGCC | | |
| | | | GCCAUCGUGAGAGU | | |
| | | | GACCAUCAACUUCU | | |
| | | | UCAGACAGCCUCAG | | |
| | | | AUGAGACACCUGAG | | |
| | | | CUGGUUCCUGGCCG | | |
| | | | GCGACUUCAACAGA | | |
| | | | AGCCCUGACAGACU | | |
| | | | GGAGAACGACCUGA | | |
| | | | UGACCGAGCACCUG | | |
| | | | GAGAGAGUGGUGGC | | |
| | | | CGUGCUGGCCCCUA | | |
| | | | CCGAGCCUACCCAG | | |
| | | | AUCGGCGGCGGCAU | | |
| | | | CCUGGACUACGGCG | | |
| | | | UGAUCGUGGACAGA | | |
| | | | GCCCCUUACAGCCA | | |
| | | | GAGAGUGGAGGCCC | | |
| | | | UGAGAAACCCUCAG | | |
| | | | CUGGCCAGCGACCA | | |
| | | | CUACCCUGUGGCCU | | |
| | | | UCCUGGCCAGAAGC | | |
| | | | UGC | | |
| SEQ ID NO: | | 157 | 158 | 3 | 4 |

SEQ ID NO: 209 consists of from 5′ end to 3′ end, 5′ UTR SEQ
ID NO: 3, mRNA ORF SEQ ID NO: 158, and 3′ UTR SEQ ID NO: 4.

| | St_CdtB_n | METPAQLLFLLLLWL | AUGGAGACUCCUGC | GGGAAA | UGAUAA |
|---|---|---|---|---|---|
| | IgK_cHis | PDTTGNISDYKVMT | CCAGCUGCUGUUCC | UAAGAG | UAGGCU |
| | | WNLQGSSASTESKW | UGCUGCUGCUGUGG | AGAAAA | GGAGCC |
| | | NVNVRQLLSGTAGV | CUGCCUGACACCAC | GAAGAG | UCGGUG |
| | | DILMVQEAGAVPTSA | CGGCAACAUCAGCG | UAAGAA | GCCAUG |
| | | VPTGRHIQPFGVGIPI | ACUACAAGGUGAUG | GAAAUA | CUUCUU |
| | | DEYTWNLGTTSRQDI | ACCUGGAACCUGCA | UAAGAG | GCCCCU |
| | | RYIYHSAIDVGARRV | GGGAAGCUCCGCCA | CCACC | UGGGCC |
| | | NLAIVSRQRADNVYV | GCACCGAGAGCAAG | | UCCCCC |
| | | LRPTTVASRPVIGIGL | UGGAACGUGAACGU | | CAGCCC |
| | | GNDVFLTAHALASG | GAGACAGCUCCUGA | | CUCCUC |
| | | GPDAAAIVRVTINFFR | GCGGCACCGCCGGC | | CCCUUC |
| | | QPQMRHLSWFLAGD | GUCGACAUCCUGAU | | CUGCAC |
| | | FNRSPDRLENDLMTE | GGUGCAGGAGGCCG | | CCGUAC |
| | | HLERVVAVLAPTEPT | GAGCCGUCCCUACC | | CCCCGU |
| | | QIGGGILDYGVIVDR | AGCGCCGUGCCUAC | | GGUCUU |
| | | APYSQRVEALRNPQL | CGGCAGACACAUCC | | UGAAUA |
| | | ASDHYPVAFLARSCH | AGCCUUUCGGCGUG | | AAGUCU |
| | | HHHHH | GGCAUCCCUAUCGA | | GAGUGG |
| | | | CGAGUACACCUGGA | | GCGGC |
| | | | AUCUCGGCACCACC | | |
| | | | AGCAGACAGGACAU | | |
| | | | CAGAUACAUCUACC | | |
| | | | ACAGCGCCAUCGAC | | |
| | | | GUGGGCGCCAGAAG | | |
| | | | AGUGAACCUGGCCA | | |
| | | | UCGUGAGCAGACAG | | |
| | | | AGAGCCGACAACGU | | |
| | | | GUACGUGCUGAGGC | | |
| | | | CUACCACCGUGGCC | | |
| | | | AGCAGACCUGUGAU | | |
| | | | CGGCAUCGGCCUGG | | |
| | | | GCAACGACGUGUUC | | |
| | | | CUGACCGCCCACGC | | |
| | | | UCUGGCCUCCGGUG | | |
| | | | GCCCCGACGCUGCC | | |
| | | | GCCAUCGUGAGAGU | | |
| | | | GACCAUCAACUUCU | | |
| | | | UCAGACAGCCUCAG | | |
| | | | AUGAGACACCUGAG | | |
| | | | CUGGUUCCUGGCCG | | |
| | | | GCGACUUCAACAGA | | |
| | | | AGCCCUGACAGACU | | |
| | | | GGAGAACGACCUGA | | |
| | | | UGACCGAGCACCUG | | |
| | | | GAGAGAGUGGUGGC | | |
| | | | CGUGCUGGCCCCUA | | |
| | | | CCGAGCCUACCCAG | | |
| | | | AUCGGCGGCGGCAU | | |
| | | | CCUGGACUACGGCG | | |
| | | | UGAUCGUGGACAGA | | |
| | | | GCCCCUUACAGCCA | | |
| | | | GAGAGUGGAGGCCC | | |
| | | | UGAGAAACCCUCAG | | |
| | | | CUGGCCAGCGACCA | | |
| | | | CUACCCUGUGGCCU | | |
| | | | UCCUGGCCAGAAGC | | |
| | | | UGCCAUCACCACCA | | |
| | | | CCACCAC | | |
| SEQ ID NO: | | 99 | 100 | 3 | 4 |

SEQ ID NO: 210 consists of from 5′ end to 3′ end, 5′ UTR SEQ
ID NO: 3, mRNA ORF SEQ ID NO: 100, and 3′ UTR SEQ ID NO: 4.

| | St_CdtB_ | METPAQLLFLLLLWL | AUGGAGACUCCUGC | GGGAAA | UGAUAA |
|---|---|---|---|---|---|
| | NGM_nIg | PDTTGNIADYKVMT | CCAGCUGCUGUUCC | UAAGAG | UAGGCU |
| | K | WNLQGSSASTESKW | UGCUGCUGCUGUGG | AGAAAA | GGAGCC |
| | | NVNVRQLLSGTAGV | CUGCCUGACACCAC | GAAGAG | UCGGUG |
| | | DILMVQEAGAVPTSA | CGGCAACAUCGCAG | UAAGAA | GCCAUG |
| | | VPTGRHIQPFGVGIPI | ACUACAAGGUGAUG | GAAAUA | CUUCUU |
| | | DEYTWNLGTTSRQDI | ACCUGGAACCUGCA | UAAGAG | GCCCCU |
| | | RYIYHSAIDVGARRV | GGGAAGCUCCGCCA | CCACC | UGGGCC |
| | | NLAIVSRQRADNVYV | GCACCGAGAGCAAG | | UCCCCC |
| | | LRPTTVASRPVIGIGL | UGGAACGUGAACGU | | CAGCCC |
| | | GNDVFLTAHALASG | GAGACAGCUCCUGA | | CUCCUC |
| | | GPDAAAIVRVTINFFR | GCGGCACCGCCGGC | | CCCUUC |
| | | QPQMRHLSWFLAGD | GUCGACAUCCUGAU | | CUGCAC |
| | | FNRSPDRLENDLMTE | GGUGCAGGAGGCCG | | CCGUAC |
| | | HLERVVAVLAPTEPT | GAGCCGUCCCUACC | | CCCCGU |
| | | QIGGGILDYGVIVDR | AGCGCCGUGCCUAC | | GGUCUU |
| | | APYSQRVEALRNPQL | CGGCAGACACAUCC | | UGAAUA |
| | | ASDHYPVAFLARSC | AGCCUUUCGGCGUG | | AAGUCU |
| | | | GGCAUCCCUAUCGA | | GAGUGG |
| | | | CGAGUACACCUGGA | | GCGGC |
| | | | AUCUCGGCACCACC | | |
| | | | AGCAGACAGGACAU | | |
| | | | CAGAUACAUCUACC | | |
| | | | ACAGCGCCAUCGAC | | |
| | | | GUGGGCGCCAGAAG | | |
| | | | AGUGAACCUGGCCA | | |
| | | | UCGUGAGCAGACAG | | |
| | | | AGAGCCGACAACGU | | |
| | | | GUACGUGCUGAGGC | | |
| | | | CUACCACCGUGGCC | | |
| | | | AGCAGACCUGUGAU | | |
| | | | CGGCAUCGGCCUGG | | |
| | | | GCAACGACGUGUUC | | |
| | | | CUGACCGCCCACGC | | |
| | | | UCUGGCCUCCGGUG | | |
| | | | GCCCCGACGCUGCC | | |
| | | | GCCAUCGUGAGAGU | | |
| | | | GACCAUCAACUUCU | | |
| | | | UCAGACAGCCUCAG | | |
| | | | AUGAGACACCUGAG | | |
| | | | CUGGUUCCUGGCCG | | |
| | | | GCGACUUCAACAGA | | |
| | | | AGCCCUGACAGACU | | |
| | | | GGAGAACGACCUGA | | |
| | | | UGACCGAGCACCUG | | |
| | | | GAGAGAGUGGUGGC | | |
| | | | CGUGCUGGCCCCUA | | |
| | | | CCGAGCCUACCCAG | | |
| | | | AUCGGCGGCGGCAU | | |
| | | | CCUGGACUACGGCG | | |
| | | | UGAUCGUGGACAGA | | |
| | | | GCCCCUUACAGCCA | | |
| | | | GAGAGUGGAGGCCC | | |
| | | | UGAGAAACCCUCAG | | |
| | | | CUGGCCAGCGACCA | | |
| | | | CUACCCUGUGGCCU | | |
| | | | UCCUGGCCAGAAGC | | |
| | | | UGC | | |
| SEQ ID NO: | | 101 | 102 | 3 | 4 |

SEQ ID NO: 211 consists of from 5′ end to 3′ end, 5′ UTR SEQ
ID NO: 3, mRNA ORF SEQ ID NO: 102, and 3′ UTR SEQ ID NO: 4.

| | St_CdtB_ | METPAQLLFLLLLWL | AUGGAGACUCCUGC | GGGAAA | UGAUAA |
|---|---|---|---|---|---|
| | NGM_nIg | PDTTGNIADYKVMT | CCAGCUGCUGUUCC | UAAGAG | UAGGCU |
| | K_cHis | WNLQGSSASTESKW | UGCUGCUGCUGUGG | AGAAAA | GGAGCC |
| | | NVNVRQLLSGTAGV | CUGCCUGACACCAC | GAAGAG | UCGGUG |
| | | DILMVQEAGAVPTSA | CGGCAACAUCGCAG | UAAGAA | GCCAUG |
| | | VPTGRHIQPFGVGIPI | ACUACAAGGUGAUG | GAAAUA | CUUCUU |
| | | DEYTWNLGTTSRQDI | ACCUGGAACCUGCA | UAAGAG | GCCCCU |
| | | RYIYHSAIDVGARRV | GGGAAGCUCCGCCA | CCACC | UGGGCC |
| | | NLAIVSRQRADNVYV | GCACCGAGAGCAAG | | UCCCCC |
| | | LRPTTVASRPVIGIGL | UGGAACGUGAACGU | | CAGCCC |
| | | GNDVFLTAHALASG | GAGACAGCUCCUGA | | CUCCUC |
| | | GPDAAAIVRVTINFFR | GCGGCACCGCCGGC | | CCCUUC |
| | | QPQMRHLSWFLAGD | GUCGACAUCCUGAU | | CUGCAC |
| | | FNRSPDRLENDLMTE | GGUGCAGGAGGCCG | | CCGUAC |
| | | HLERVVAVLAPTEPT | GAGCCGUCCCUACC | | CCCCGU |
| | | QIGGGILDYGVIVDR | AGCGCCGUGCCUAC | | GGUCUU |
| | | APYSQRVEALRNPQL | CGGCAGACACAUCC | | UGAAUA |
| | | ASDHYPVAFLARSCH | AGCCUUUCGGCGUG | | AAGUCU |
| | | HHHHH | GGCAUCCCUAUCGA | | GAGUGG |
| | | | CGAGUACACCUGGA | | GCGGC |
| | | | AUCUCGGCACCACC | | |
| | | | AGCAGACAGGACAU | | |
| | | | CAGAUACAUCUACC | | |
| | | | ACAGCGCCAUCGAC | | |
| | | | GUGGGCGCCAGAAG | | |
| | | | AGUGAACCUGGCCA | | |
| | | | UCGUGAGCAGACAG | | |
| | | | AGAGCCGACAACGU | | |
| | | | CUACCACCGUGGCC | | |
| | | | AGCAGACCUGUGAU | | |
| | | | CGGCAUCGGCCUGG | | |
| | | | GCAACGACGUGUUC | | |
| | | | CUGACCGCCCACGC | | |
| | | | UCUGGCCUCCGGUG | | |
| | | | GCCCCGACGCUGCC | | |
| | | | GCCAUCGUGAGAGU | | |
| | | | GACCAUCAACUUCU | | |
| | | | UCAGACAGCCUCAG | | |
| | | | AUGAGACACCUGAG | | |
| | | | CUGGUUCCUGGCCG | | |
| | | | GCGACUUCAACAGA | | |
| | | | AGCCCUGACAGACU | | |
| | | | GGAGAACGACCUGA | | |
| | | | UGACCGAGCACCUG | | |
| | | | GAGAGAGUGGUGGC | | |
| | | | CGUGCUGGCCCCUA | | |
| | | | CCGAGCCUACCCAG | | |
| | | | AUCGGCGGCGGCAU | | |
| | | | CCUGGACUACGGCG | | |
| | | | UGAUCGUGGACAGA | | |
| | | | GCCCCUUACAGCCA | | |
| | | | GAGAGUGGAGGCCC | | |
| | | | UGAGAAACCCUCAG | | |
| | | | CUGGCCAGCGACCA | | |
| | | | CUACCCUGUGGCCU | | |
| | | | UCCUGGCCAGAAGC | | |
| | | | UGCCAUCACCACCA | | |
| | | | CCACCAC | | |
| SEQ ID NO: | | 103 | 104 | 3 | 4 |

SEQ ID NO: 212 consists of from 5′ end to 3′ end, 5′ UTR SEQ
ID NO: 3, mRNA ORF SEQ ID NO: 104, and 3′ UTR SEQ ID NO: 4.

| | St_OmpL_ | METPAQLLFLLLLWL | AUGGAGACGCCUGC | GGGAAA | UGAUAA |
|---|---|---|---|---|---|
| | nIgK | PDTTGGAYVENREA | CCAGCUGCUGUUCC | UAAGAG | UAGGCU |
| | | YNLASDQMEFMLRV | UGCUCCUUCUGUGG | AGAAAA | GGAGCC |
| | | GYNSDMGAGIMLTN | CUGCCUGACACCAC | GAAGAG | UCGGUG |
| | | TYTLQRDDELKHGY | CGGCGGCGCCUACG | UAAGAA | GCCAUG |
| | | NEIEGWYPLFKPTDK | UGGAGAACAGAGAG | GAAAUA | CUUCUU |
| | | LTIQPGGLINDKSIGS | GCCUACAACCUGGC | UAAGAG | GCCCCU |
| | | GGAVYLDINYKFTP | CAGCGACCAGAUGG | CCACC | UGGGCC |
| | | WFNLTVRNRYNHNN | AGUUCAUGCUGAGA | | UCCCCC |
| | | YSSTDLNGELDNNDS | GUGGGCUACAACAG | | CAGCCC |
| | | YEIGNYWNFIITDKFS | CGACAUGGGCGCCG | | CUCCUC |
| | | YTFEPHYFYNVNDFN | GCAUCAUGCUUACC | | CCCUUC |
| | | SSNGTKHHWEITNTF | AACACCUACACCCU | | CUGCAC |
| | | RYRINEHWLPYFELR | GCAGAGAGACGACG | | CCGUAC |
| | | WLDRNVGLYHREQN | AGCUGAAGCACGGC | | CCCCGU |
| | | QIRIGAKYFF | UACAACGAGAUCGA | | GGUCUU |
| | | | GGGCUGGUACCCUC | | UGAAUA |
| | | | UGUUCAAGCCUACC | | AAGUCU |
| | | | GACAAGCUGACCAU | | GAGUGG |
| | | | CCAGCCUGGCGGCC | | GCGGC |
| | | | UGAUCAACGACAAG | | |
| | | | AGCAUCGGCUCUGG | | |
| | | | CGGUGCCGUGUACC | | |
| | | | UGGACAUCAACUAC | | |
| | | | AAGUUCACCCCUUG | | |
| | | | GUUCAACCUGACCG | | |
| | | | UGAGAAACAGAUAC | | |
| | | | AACCACAACAACUA | | |
| | | | CAGCAGCACCGACC | | |
| | | | UGAACGGCGAGCUG | | |
| | | | GACAACAACGACAG | | |
| | | | CUACGAGAUUGGCA | | |
| | | | ACUACUGGAACUUC | | |
| | | | AUCAUCACCGAUAA | | |
| | | | GUUCAGCUACACCU | | |
| | | | UCGAGCCUCACUAC | | |
| | | | UUCUACAACGUGAA | | |
| | | | CGACUUCAAUAGUA | | |
| | | | GCAACGGCACCAAG | | |
| | | | CACCACUGGGAGAU | | |
| | | | CACCAACACGUUCA | | |
| | | | GAUACAGAAUCAAC | | |
| | | | GAGCACUGGCUUCC | | |
| | | | UUACUUCGAGCUGA | | |
| | | | GGUGGCUGGACAGA | | |
| | | | AACGUGGGCCUGUA | | |
| | | | CCACAGAGAGCAGA | | |
| | | | ACCAGAUCAGAAUC | | |
| | | | GGCGCCAAGUACUU | | |
| | | | CUUC | | |
| SEQ ID NO: | | 105 | 106 | 3 | 4 |

SEQ ID NO: 213 consists of from 5′ end to 3′ end, 5′ UTR SEQ
ID NO: 3, mRNA ORF SEQ ID NO: 106, and 3′ UTR SEQ ID NO: 4.

| | St_OmpL_ | METPAQLLFLLLLWL | AUGGAGACGCCUGC | GGGAAA | UGAUAA |
|---|---|---|---|---|---|
| | nIgK_cHis | PDTTGGAYVENREA | CCAGCUGCUGUUCC | UAAGAG | UAGGCU |
| | | YNLASDQMEFMLRV | UGCUCCUUCUGUGG | AGAAAA | GGAGCC |
| | | GYNSDMGAGIMLTN | CUGCCUGACACCAC | GAAGAG | UCGGUG |
| | | TYTLQRDDELKHGY | CGGCGGCGCCUACG | UAAGAA | GCCAUG |
| | | NEIEGWYPLFKPTDK | UGGAGAACAGAGAG | GAAAUA | CUUCUU |
| | | LTIQPGGLINDKSIGS | GCCUACAACCUGGC | UAAGAG | GCCCCU |
| | | GGAVYLDINYKFTP | CAGCGACCAGAUGG | CCACC | UGGGCC |
| | | WFNLTVRNRYNHNN | AGUUCAUGCUGAGA | | UCCCCC |
| | | YSSTDLNGELDNNDS | GUGGGCUACAACAG | | CAGCCC |
| | | YEIGNYWNFIITDKFS | CGACAUGGGCGCCG | | CUCCUC |
| | | YTFEPHYFYNVNDFN | GCAUCAUGCUUACC | | CCCUUC |
| | | SSNGTKHHWEITNTF | AACACCUACACCCU | | CUGCAC |
| | | RYRINEHWLPYFELR | GCAGAGAGACGACG | | CCGUAC |
| | | WLDRNVGLYHREQN | AGCUGAAGCACGGC | | CCCCGU |
| | | QIRIGAKYFFHHHHH | UACAACGAGAUCGA | | GGUCUU |
| | | H | GGGCUGGUACCCUC | | UGAAUA |
| | | | UGUUCAAGCCUACC | | AAGUCU |
| | | | GACAAGCUGACCAU | | GAGUGG |
| | | | CCAGCCUGGCGGCC | | GCGGC |
| | | | UGAUCAACGACAAG | | |
| | | | AGCAUCGGCUCUGG | | |
| | | | CGGUGCCGUGUACC | | |
| | | | UGGACAUCAACUAC | | |
| | | | AAGUUCACCCCUUG | | |
| | | | GUUCAACCUGACCG | | |
| | | | UGAGAAACAGAUAC | | |
| | | | AACCACAACAACUA | | |
| | | | CAGCAGCACCGACC | | |
| | | | UGAACGGCGAGCUG | | |
| | | | GACAACAACGACAG | | |
| | | | CUACGAGAUUGGCA | | |
| | | | ACUACUGGAACUUC | | |
| | | | AUCAUCACCGAUAA | | |
| | | | GUUCAGCUACACCU | | |
| | | | UCGAGCCUCACUAC | | |
| | | | UUCUACAACGUGAA | | |
| | | | CGACUUCAAUAGUA | | |
| | | | GCAACGGCACCAAG | | |
| | | | CACCACUGGGAGAU | | |
| | | | CACCAACACGUUCA | | |
| | | | GAUACAGAAUCAAC | | |
| | | | GAGCACUGGCUUCC | | |
| | | | UUACUUCGAGCUGA | | |
| | | | GGUGGCUGGACAGA | | |
| | | | AACGUGGGCCUGUA | | |
| | | | CCACAGAGAGCAGA | | |
| | | | ACCAGAUCAGAAUC | | |
| | | | GGCGCCAAGUACUU | | |
| | | | CUUCCACCAUCACC | | |
| | | | ACCACCAC | | |
| SEQ ID NO: | | 107 | 108 | 3 | 4 |

SEQ ID NO: 214 consists of from 5′ end to 3′ end, 5′ UTR SEQ
ID NO: 3, mRNA ORF SEQ ID NO: 108, and 3′ UTR SEQ ID NO: 4.

| | St_OmpL_ | METPAQLLFLLLLWL | AUGGAGACGCCUGC | GGGAAA | UGAUAA |
|---|---|---|---|---|---|
| | NGM_nIg | PDTTGGAYVENREA | CCAGCUGCUGUUCC | UAAGAG | UAGGCU |
| | K | YNLASDQMEFMLRV | UGCUCCUUCUGUGG | AGAAAA | GGAGCC |
| | | GYNSDMGAGIMLTN | CUGCCUGACACCAC | GAAGAG | UCGGUG |
| | | TYTLQRDDELKHGY | CGGCGGCGCCUACG | UAAGAA | GCCAUG |
| | | NEIEGWYPLFKPTDK | UGGAGAACAGAGAG | GAAAUA | CUUCUU |
| | | LTIQPGGLINDKSIGS | GCCUACAACCUGGC | UAAGAG | GCCCCU |
| | | GGAVYLDINYKFTP | CAGCGACCAGAUGG | CCACC | UGGGCC |
| | | WFNLAVRNRYNHNN | AGUUCAUGCUGAGA | | UCCCCC |
| | | YASTDLNGELDNNDS | GUGGGCUACAACAG | | CAGCCC |
| | | YEIGNYWNFIITDKFS | CGACAUGGGCGCCG | | CUCCUC |
| | | YTFEPHYFYNVNDFN | GCAUCAUGCUUACC | | CCCUUC |
| | | SSNGAKHHWEITNTF | AACACCUACACCCU | | CUGCAC |
| | | RYRINEHWLPYFELR | GCAGAGAGACGACG | | CCGUAC |
| | | WLDRNVGLYHREQN | AGCUGAAGCACGGC | | CCCCGU |
| | | QIRIGAKYFF | UACAACGAGAUCGA | | GGUCUU |
| | | | GGGCUGGUACCCUC | | UGAAUA |
| | | | UGUUCAAGCCUACC | | AAGUCU |
| | | | GACAAGCUGACCAU | | GAGUGG |
| | | | CCAGCCUGGCGGCC | | GCGGC |
| | | | UGAUCAACGACAAG | | |
| | | | AGCAUCGGCUCUGG | | |
| | | | CGGUGCCGUGUACC | | |
| | | | UGGACAUCAACUAC | | |
| | | | AAGUUCACCCCUUG | | |
| | | | GUUCAACCUGGCCG | | |
| | | | UGAGAAACAGAUAC | | |
| | | | AACCACAACAACUA | | |
| | | | CGCAAGCACCGACC | | |
| | | | UGAACGGCGAGCUG | | |
| | | | GACAACAACGACAG | | |
| | | | CUACGAGAUUGGCA | | |
| | | | ACUACUGGAACUUC | | |
| | | | AUCAUCACCGAUAA | | |
| | | | GUUCAGCUACACCU | | |
| | | | UCGAGCCUCACUAC | | |
| | | | UUCUACAACGUGAA | | |
| | | | CGACUUCAAUAGUA | | |
| | | | GCAACGGCGCCAAG | | |
| | | | CACCACUGGGAGAU | | |
| | | | CACCAACACGUUCA | | |
| | | | GAUACAGAAUCAAC | | |
| | | | GAGCACUGGCUUCC | | |
| | | | UUACUUCGAGCUGA | | |
| | | | GGUGGCUGGACAGA | | |
| | | | AACGUGGGCCUGUA | | |
| | | | CCACAGAGAGCAGA | | |
| | | | ACCAGAUCAGAAUC | | |
| | | | GGCGCCAAGUACUU | | |
| | | | CUUC | | |
| SEQ ID NO: | | 109 | 110 | 3 | 4 |

SEQ ID NO: 215 consists of from 5′ end to 3′ end, 5′ UTR SEQ
ID NO: 3, mRNA ORF SEQ ID NO: 110, and 3′ UTR SEQ ID NO: 4.

| | St_OmpL_ | METPAQLLFLLLLWL | AUGGAGACGCCUGC | GGGAAA | UGAUAA |
|---|---|---|---|---|---|
| | NGM_nIg | PDTTGGAYVENREA | CCAGCUGCUGUUCC | UAAGAG | UAGGCU |
| | K_cHis | YNLASDQMEFMLRV | UGCUCCUUCUGUGG | AGAAAA | GGAGCC |
| | | GYNSDMGAGIMLTN | CUGCCUGACACCAC | GAAGAG | UCGGUG |
| | | TYTLQRDDELKHGY | CGGCGGCGCCUACG | UAAGAA | GCCAUG |
| | | NEIEGWYPLFKPTDK | UGGAGAACAGAGAG | GAAAUA | CUUCUU |
| | | LTIQPGGLINDKSIGS | GCCUACAACCUGGC | UAAGAG | GCCCCU |
| | | GGAVYLDINYKFTP | CAGCGACCAGAUGG | CCACC | UGGGCC |
| | | WFNLAVRNRYNHNN | AGUUCAUGCUGAGA | | UCCCCC |
| | | YASTDLNGELDNNDS | GUGGGCUACAACAG | | CAGCCC |
| | | YEIGNYWNFIITDKFS | CGACAUGGGCGCCG | | CUCCUC |
| | | YTFEPHYFYNVNDFN | GCAUCAUGCUUACC | | CCCUUC |
| | | SSNGAKHHWEITNTF | AACACCUACACCCU | | CUGCAC |
| | | RYRINEHWLPYFELR | GCAGAGAGACGACG | | CCGUAC |
| | | WLDRNVGLYHREQN | AGCUGAAGCACGGC | | CCCCGU |
| | | QIRIGAKYFFHHHHH | UACAACGAGAUCGA | | GGUCUU |
| | | H | GGGCUGGUACCCUC | | UGAAUA |
| | | | UGUUCAAGCCUACC | | AAGUCU |
| | | | GACAAGCUGACCAU | | GAGUGG |
| | | | CCAGCCUGGCGGCC | | GCGGC |
| | | | UGAUCAACGACAAG | | |
| | | | AGCAUCGGCUCUGG | | |
| | | | CGGUGCCGUGUACC | | |
| | | | UGGACAUCAACUAC | | |
| | | | AAGUUCACCCCUUG | | |
| | | | GUUCAACCUGGCCG | | |
| | | | UGAGAAACAGAUAC | | |
| | | | AACCACAACAACUA | | |
| | | | CGCAAGCACCGACC | | |
| | | | UGAACGGCGAGCUG | | |
| | | | GACAACAACGACAG | | |
| | | | CUACGAGAUUGGCA | | |
| | | | ACUACUGGAACUUC | | |
| | | | AUCAUCACCGAUAA | | |
| | | | GUUCAGCUACACCU | | |
| | | | UCGAGCCUCACUAC | | |
| | | | UUCUACAACGUGAA | | |
| | | | CGACUUCAAUAGUA | | |
| | | | GCAACGGCGCCAAG | | |
| | | | CACCACUGGGAGAU | | |
| | | | CACCAACACGUUCA | | |
| | | | GAUACAGAAUCAAC | | |
| | | | GAGCACUGGCUUCC | | |
| | | | UUACUUCGAGCUGA | | |
| | | | GGUGGCUGGACAGA | | |
| | | | AACGUGGGCCUGUA | | |
| | | | CCACAGAGAGCAGA | | |
| | | | ACCAGAUCAGAAUC | | |
| | | | GGCGCCAAGUACUU | | |
| | | | CUUCCACCAUCACC | | |
| | | | ACCACCAC | | |
| SEQ ID NO: | | 111 | 112 | 3 | 4 |

SEQ ID NO: 216 consists of from 5′ end to 3′ end, 5′ UTR SEQ
ID NO: 3, mRNA ORF SEQ ID NO: 112, and 3′ UTR SEQ ID NO: 4.

| | St_PltA_nI | METPAQLLFLLLLWL | AUGGAAACCCCUGC | GGGAAA | UGAUAA |
|---|---|---|---|---|---|
| | gK | PDTTGVDFVYRVDST | CCAGCUGCUGUUCC | UAAGAG | UAGGCU |
| | | PPDVIFRDGFSLLGYN | UGCUGCUGCUCUGG | AGAAAA | GGAGCC |
| | | RNFQQFISGRSCSGGS | CUGCCUGACACAAC | GAAGAG | UCGGUG |
| | | SDSRYIATTSSVNQT | CGGCGUGGACUUCG | UAAGAA | GCCAUG |
| | | YAIARAYYSRSTFKG | UGUACAGAGUGGAC | GAAAUA | CUUCUU |
| | | NLYRYQIRADNNFYS | AGCACCCCUCCUGA | UAAGAG | GCCCCU |
| | | LLPSITYLETQGGHFN | CGUGAUCUUCAGAG | CCACC | UGGGCC |
| | | AYEKTMMRLQREYV | ACGGCUUCAGCCUG | | UCCCCC |
| | | STLSILPENIQKAVAL | CUGGGCUACAACAG | | CAGCCC |
| | | VYDSATGLVKDGVS | AAACUUCCAGCAGU | | CUCCUC |
| | | TMNASYLGLSTTSNP | UCAUCAGCGGCAGA | | CCCUUC |
| | | GVIPFLPEPQTYTQQR | AGCUGCAGCGGCGG | | CUGCAC |
| | | IDAFGPLISSCFSIGSV | UAGCAGCGAUAGCA | | CCGUAC |
| | | CHSHRGQRADVYNM | GAUACAUCGCCACC | | CCCCGU |
| | | SFYDARPVIELILSK | ACCAGCAGCGUGAA | | GGUCUU |
| | | | CCAGACCUACGCCA | | UGAAUA |
| | | | UCGCCAGAGCCUAC | | AAGUCU |
| | | | UACAGCAGAAGCAC | | GAGUGG |
| | | | CUUCAAGGGCAACC | | GCGGC |
| | | | UGUACAGAUACCAG | | |
| | | | AUCAGAGCCGACAA | | |
| | | | CAACUUCUACAGCC | | |
| | | | UGCUGCCUAGCAUC | | |
| | | | ACCUACCUGGAAAC | | |
| | | | GCAGGGCGGCCACU | | |
| | | | UCAACGCCUACGAG | | |
| | | | AAGACCAUGAUGAG | | |
| | | | ACUGCAGAGAGAGU | | |
| | | | ACGUGAGCACCCUG | | |
| | | | UCAAUCCUGCCCGA | | |
| | | | GAACAUCCAGAAGG | | |
| | | | CCGUGGCCCUGGUG | | |
| | | | UACGACAGCGCCAC | | |
| | | | CGGCCUGGUGAAGG | | |
| | | | ACGGCGUGAGCACC | | |
| | | | AUGAACGCCAGCUA | | |
| | | | CCUCGGGCUGAGCA | | |
| | | | CAACCAGCAACCCU | | |
| | | | GGCGUGAUCCCUUU | | |
| | | | CCUGCCUGAGCCUC | | |
| | | | AGACCUACACCCAG | | |
| | | | CAGAGAAUCGACGC | | |
| | | | CUUCGGCCCUCUGA | | |
| | | | UCAGCAGCUGCUUC | | |
| | | | AGCAUCGGCAGCGU | | |
| | | | GUGCCACAGCCACA | | |
| | | | GAGGCCAGAGAGCC | | |
| | | | GACGUGUACAACAU | | |
| | | | GAGCUUCUACGACG | | |
| | | | CCAGACCUGUGAUC | | |
| | | | GAGCUGAUCCUGUC | | |
| | | | CAAG | | |
| SEQ ID NO: | | 113 | 114 | 3 | 4 |

SEQ ID NO: 217 consists of from 5′ end to 3′ end, 5′ UTR SEQ
ID NO: 3, mRNA ORF SEQ ID NO: 114, and 3′ UTR SEQ ID NO: 4.

| | St_PltA_nI | METPAQLLFLLLLWL | AUGGAAACCCCUGC | GGGAAA | UGAUAA |
|---|---|---|---|---|---|
| | gK_cHis | PDTTGVDFVYRVDST | CCAGCUGCUGUUCC | UAAGAG | UAGGCU |
| | | PPDVIFRDGFSLLGYN | UGCUGCUGCUCUGG | AGAAAA | GGAGCC |
| | | RNFQQFISGRSCSGGS | CUGCCUGACACAAC | GAAGAG | UCGGUG |
| | | SDSRYIATTSSVNQT | CGGCGUGGACUUCG | UAAGAA | GCCAUG |
| | | YAIARAYYSRSTFKG | UGUACAGAGUGGAC | GAAAUA | CUUCUU |
| | | NLYRYQIRADNNFYS | AGCACCCCUCCUGA | UAAGAG | GCCCCU |
| | | LLPSITYLETQGGHFN | CGUGAUCUUCAGAG | CCACC | UGGGCC |
| | | AYEKTMMRLQREYV | ACGGCUUCAGCCUG | | UCCCCC |
| | | STLSILPENIQKAVAL | CUGGGCUACAACAG | | CAGCCC |
| | | VYDSATGLVKDGVS | AAACUUCCAGCAGU | | CUCCUC |
| | | TMNASYLGLSTTSNP | UCAUCAGCGGCAGA | | CCCUUC |
| | | GVIPFLPEPQTYTQQR | AGCUGCAGCGGCGG | | CUGCAC |
| | | IDAFGPLISSCFSIGSV | UAGCAGCGAUAGCA | | CCGUAC |
| | | CHSHRGQRADVYNM | GAUACAUCGCCACC | | CCCCGU |
| | | SFYDARPVIELILSKH | ACCAGCAGCGUGAA | | GGUCUU |
| | | HHHHH | CCAGACCUACGCCA | | UGAAUA |
| | | | UCGCCAGAGCCUAC | | AAGUCU |
| | | | UACAGCAGAAGCAC | | GAGUGG |
| | | | CUUCAAGGGCAACC | | GCGGC |
| | | | UGUACAGAUACCAG | | |
| | | | AUCAGAGCCGACAA | | |
| | | | CAACUUCUACAGCC | | |
| | | | UGCUGCCUAGCAUC | | |
| | | | ACCUACCUGGAAAC | | |
| | | | GCAGGGCGGCCACU | | |
| | | | UCAACGCCUACGAG | | |
| | | | AAGACCAUGAUGAG | | |
| | | | ACUGCAGAGAGAGU | | |
| | | | ACGUGAGCACCCUG | | |
| | | | UCAAUCCUGCCCGA | | |
| | | | GAACAUCCAGAAGG | | |
| | | | CCGUGGCCCUGGUG | | |
| | | | UACGACAGCGCCAC | | |
| | | | CGGCCUGGUGAAGG | | |
| | | | ACGGCGUGAGCACC | | |
| | | | AUGAACGCCAGCUA | | |
| | | | CCUCGGGCUGAGCA | | |
| | | | CAACCAGCAACCCU | | |
| | | | GGCGUGAUCCCUUU | | |
| | | | CCUGCCUGAGCCUC | | |
| | | | AGACCUACACCCAG | | |
| | | | CAGAGAAUCGACGC | | |
| | | | CUUCGGCCCUCUGA | | |
| | | | UCAGCAGCUGCUUC | | |
| | | | AGCAUCGGCAGCGU | | |
| | | | GUGCCACAGCCACA | | |
| | | | GAGGCCAGAGAGCC | | |
| | | | GACGUGUACAACAU | | |
| | | | GAGCUUCUACGACG | | |
| | | | CCAGACCUGUGAUC | | |
| | | | GAGCUGAUCCUGUC | | |
| | | | CAAGCACCACCACC | | |
| | | | AUCACCAC | | |
| SEQ ID NO: | | 115 | 116 | 3 | 4 |

SEQ ID NO: 218 consists of from 5′ end to 3′ end, 5′ UTR SEQ
ID NO: 3, mRNA ORF SEQ ID NO: 116, and 3′ UTR SEQ ID NO: 4.

| | St_PltA | METPAQLLFLLLLWL | AUGGAAACCCCUGC | GGGAAA | UGAUAA |
|---|---|---|---|---|---|
| | _NGM_nI | PDTTGVDFVYRVDST | CCAGCUGCUGUUCC | UAAGAG | UAGGCU |
| | gK | PPDVIFRDGFSLLGYN | UGCUGCUGCUCUGG | AGAAAA | GGAGCC |
| | | RNFQQFISGRSCSGGS | CUGCCUGACACAAC | GAAGAG | UCGGUG |
| | | SDSRYIATTSSVNQA | CGGCGUGGACUUCG | UAAGAA | GCCAUG |
| | | YAIARAYYSRSTFKG | UGUACAGAGUGGAC | GAAAUA | CUUCUU |
| | | NLYRYQIRADNNFYS | AGCACCCCUCCUGA | UAAGAG | GCCCCU |
| | | LLPSITYLETQGGHFN | CGUGAUCUUCAGAG | CCACC | UGGGCC |
| | | AYEKTMMRLQREYV | ACGGCUUCAGCCUG | | UCCCCC |
| | | STLSILPENIQKAVAL | CUGGGCUACAACAG | | CAGCCC |
| | | VYDSATGLVKDGVS | AAACUUCCAGCAGU | | CUCCUC |
| | | TMNASYLGLSTTSNP | UCAUCAGCGGCAGA | | CCCUUC |
| | | GVIPFLPEPQTYTQQR | AGCUGCAGCGGCGG | | CUGCAC |
| | | IDAFGPLISSCFSIGSV | UAGCAGCGAUAGCA | | CCGUAC |
| | | CHSHRGQRADVYNM | GAUACAUCGCCACC | | CCCCGU |
| | | SFYDARPVIELILSK | ACCAGCAGCGUGAA | | GGUCUU |
| | | | CCAGGCCUACGCCA | | UGAAUA |
| | | | UCGCCAGAGCCUAC | | AAGUCU |
| | | | UACAGCAGAAGCAC | | GAGUGG |
| | | | CUUCAAGGGCAACC | | GCGGC |
| | | | UGUACAGAUACCAG | | |
| | | | AUCAGAGCCGACAA | | |
| | | | CAACUUCUACAGCC | | |
| | | | UGCUGCCUAGCAUC | | |
| | | | ACCUACCUGGAAAC | | |
| | | | GCAGGGCGGCCACU | | |
| | | | UCAACGCCUACGAG | | |
| | | | AAGACCAUGAUGAG | | |
| | | | ACUGCAGAGAGAGU | | |
| | | | ACGUGAGCACCCUG | | |
| | | | UCAAUCCUGCCCGA | | |
| | | | GAACAUCCAGAAGG | | |
| | | | CCGUGGCCCUGGUG | | |
| | | | UACGACAGCGCCAC | | |
| | | | CGGCCUGGUGAAGG | | |
| | | | ACGGCGUGAGCACC | | |
| | | | AUGAACGCCAGCUA | | |
| | | | CCUCGGGCUGAGCA | | |
| | | | CAACCAGCAACCCU | | |
| | | | GGCGUGAUCCCUUU | | |
| | | | CCUGCCUGAGCCUC | | |
| | | | AGACCUACACCCAG | | |
| | | | CAGAGAAUCGACGC | | |
| | | | CUUCGGCCCUCUGA | | |
| | | | UCAGCAGCUGCUUC | | |
| | | | AGCAUCGGCAGCGU | | |
| | | | GUGCCACAGCCACA | | |
| | | | GAGGCCAGAGAGCC | | |
| | | | GACGUGUACAACAU | | |
| | | | GAGCUUCUACGACG | | |
| | | | CCAGACCUGUGAUC | | |
| | | | GAGCUGAUCCUGUC | | |
| | | | CAAG | | |
| SEQ ID NO: | | 117 | 118 | 3 | 4 |

SEQ ID NO: 219 consists of from 5′ end to 3′ end, 5′ UTR SEQ
ID NO: 3, mRNA ORF SEQ ID NO: 118, and 3′ UTR SEQ ID NO: 4.

| | St_PltA | METPAQLLFLLLLWL | AUGGAAACCCCUGC | GGGAAA | UGAUAA |
|---|---|---|---|---|---|
| | _NGM_nI | PDTTGVDFVYRVDST | CCAGCUGCUGUUCC | UAAGAG | UAGGCU |
| | gK_cHis | PPDVIFRDGFSLLGYN | UGCUGCUGCUCUGG | AGAAAA | GGAGCC |
| | | RNFQQFISGRSCSGGS | CUGCCUGACACAAC | GAAGAG | UCGGUG |
| | | SDSRYIATTSSVNQA | CGGCGUGGACUUCG | UAAGAA | GCCAUG |
| | | YAIARAYYSRSTFKG | UGUACAGAGUGGAC | GAAAUA | CUUCUU |
| | | NLYRYQIRADNNFYS | AGCACCCCUCCUGA | UAAGAG | GCCCCU |
| | | LLPSITYLETQGGHFN | CGUGAUCUUCAGAG | CCACC | UGGGCC |
| | | AYEKTMMRLQREYV | ACGGCUUCAGCCUG | | UCCCCC |
| | | STLSILPENIQKAVAL | CUGGGCUACAACAG | | CAGCCC |
| | | VYDSATGLVKDGVS | AAACUUCCAGCAGU | | CUCCUC |
| | | TMNASYLGLSTTSNP | UCAUCAGCGGCAGA | | CCCUUC |
| | | GVIPFLPEPQTYTQQR | AGCUGCAGCGGCGG | | CUGCAC |
| | | IDAFGPLISSCFSIGSV | UAGCAGCGAUAGCA | | CCGUAC |
| | | CHSHRGQRADVYNM | GAUACAUCGCCACC | | CCCCGU |
| | | SFYDARPVIELILSKH | ACCAGCAGCGUGAA | | GGUCUU |
| | | HHHHH | CCAGGCCUACGCCA | | UGAAUA |
| | | | UCGCCAGAGCCUAC | | AAGUCU |
| | | | UACAGCAGAAGCAC | | GAGUGG |
| | | | CUUCAAGGGCAACC | | GCGGC |
| | | | UGUACAGAUACCAG | | |
| | | | AUCAGAGCCGACAA | | |
| | | | CAACUUCUACAGCC | | |
| | | | UGCUGCCUAGCAUC | | |
| | | | ACCUACCUGGAAAC | | |
| | | | GCAGGGCGGCCACU | | |
| | | | UCAACGCCUACGAG | | |
| | | | AAGACCAUGAUGAG | | |
| | | | ACUGCAGAGAGAGU | | |
| | | | ACGUGAGCACCCUG | | |
| | | | UCAAUCCUGCCCGA | | |
| | | | GAACAUCCAGAAGG | | |
| | | | CCGUGGCCCUGGUG | | |
| | | | UACGACAGCGCCAC | | |
| | | | CGGCCUGGUGAAGG | | |
| | | | ACGGCGUGAGCACC | | |
| | | | AUGAACGCCAGCUA | | |
| | | | CCUCGGGCUGAGCA | | |
| | | | CAACCAGCAACCCU | | |
| | | | GGCGUGAUCCCUUU | | |
| | | | CCUGCCUGAGCCUC | | |
| | | | AGACCUACACCCAG | | |
| | | | CAGAGAAUCGACGC | | |
| | | | CUUCGGCCCUCUGA | | |
| | | | UCAGCAGCUGCUUC | | |
| | | | AGCAUCGGCAGCGU | | |
| | | | GUGCCACAGCCACA | | |
| | | | GACGUGUACAACAU | | |
| | | | GAGCUUCUACGACG | | |
| | | | CCAGACCUGUGAUC | | |
| | | | GAGCUGAUCCUGUC | | |
| | | | CAAGCACCACCACC | | |
| | | | AUCACCAC | | |
| SEQ ID NO: | | 119 | 120 | 3 | 4 |

SEQ ID NO: 220 consists of from 5′ end to 3′ end, 5′ UTR SEQ
ID NO: 3, mRNA ORF SEQ ID NO: 120, and 3′ UTR SEQ ID NO: 4.

| | St_PltB_nI | METPAQLLFLLLLWL | AUGGAGACACCUGC | GGGAAA | UGAUAA |
|---|---|---|---|---|---|
| | gK | PDTTGEWTGDNTNA | CCAGCUGCUGUUCC | UAAGAG | UAGGCU |
| | | YYSDEVISELHVGQI | UGCUGCUGCUGUGG | AGAAAA | GGAGCC |
| | | DTSPYFCIKTVKANG | CUGCCUGACACCAC | GAAGAG | UCGGUG |
| | | SGTPVVACAVSKQSI | UGGCGAGUGGACCG | UAAGAA | GCCAUG |
| | | WAPSFKELLDQARYF | GCGACAACACCAAC | GAAAUA | CUUCUU |
| | | YSTGQSVRIHVQKNI | GCCUACUACAGCGA | UAAGAG | GCCCCU |
| | | WTYPLFVNTFSANAL | CGAGGUGAUCUCCG | CCACC | UGGGCC |
| | | VGLSSCSATQCFGPK | AGCUGCACGUGGGA | | UCCCCC |
| | | | CAGAUCGACACCAG | | CAGCCC |
| | | | CCCUUACUUCUGCA | | CUCCUC |
| | | | UCAAGACCGUGAAG | | CCCUUC |
| | | | GCCAACGGCAGCGG | | CUGCAC |
| | | | CACCCCUGUGGUGG | | CCGUAC |
| | | | CCUGCGCCGUGAGC | | CCCCGU |
| | | | AAGCAGAGCAUCUG | | GGUCUU |
| | | | GGCCCCUAGCUUCA | | UGAAUA |
| | | | AGGAGCUGCUGGAC | | AAGUCU |
| | | | CAGGCCAGAUACUU | | GAGUGG |
| | | | CUACAGCACCGGCC | | GCGGC |
| | | | AGAGCGUGAGAAUC | | |
| | | | CACGUGCAGAAGAA | | |
| | | | CAUCUGGACCUACC | | |
| | | | CUCUGUUCGUGAAC | | |
| | | | ACCUUCAGCGCGAA | | |
| | | | CGCCCUGGUGGGCC | | |
| | | | UGAGCAGCUGCAGC | | |
| | | | GCCACCCAGUGCUU | | |
| | | | CGGCCCCAAG | | |
| SEQ ID NO: | | 121 | 122 | 3 | 4 |

SEQ ID NO: 221 consists of from 5′ end to 3′ end, 5′ UTR SEQ
ID NO: 3, mRNA ORF SEQ ID NO: 122, and 3′ UTR SEQ ID NO: 4.

| | St_PltB_nI | METPAQLLFLLLLWL | AUGGAGACACCUGC | GGGAAA | UGAUAA |
|---|---|---|---|---|---|
| | gK_cHis | PDTTGEWTGDNTNA | CCAGCUGCUGUUCC | UAAGAG | UAGGCU |
| | | YYSDEVISELHVGQI | UGCUGCUGCUGUGG | AGAAAA | GGAGCC |
| | | DTSPYFCIKTVKANG | CUGCCUGACACCAC | GAAGAG | UCGGUG |
| | | SGTPVVACAVSKQSI | UGGCGAGUGGACCG | UAAGAA | GCCAUG |
| | | WAPSFKELLDQARYF | GCGACAACACCAAC | GAAAUA | CUUCUU |
| | | YSTGQSVRIHVQKNI | GCCUACUACAGCGA | UAAGAG | GCCCCU |
| | | WTYPLFVNTFSANAL | CGAGGUGAUCUCCG | CCACC | UGGGCC |
| | | VGLSSCSATQCFGPK | AGCUGCACGUGGGA | | UCCCCC |
| | | HHHHHH | CAGAUCGACACCAG | | CAGCCC |
| | | | CCCUUACUUCUGCA | | CUCCUC |
| | | | UCAAGACCGUGAAG | | CCCUUC |
| | | | GCCAACGGCAGCGG | | CUGCAC |
| | | | CACCCCUGUGGUGG | | CCGUAC |
| | | | CCUGCGCCGUGAGC | | CCCCGU |
| | | | AAGCAGAGCAUCUG | | GGUCUU |
| | | | GGCCCCUAGCUUCA | | UGAAUA |
| | | | AGGAGCUGCUGGAC | | AAGUCU |
| | | | CAGGCCAGAUACUU | | GAGUGG |
| | | | CUACAGCACCGGCC | | GCGGC |
| | | | AGAGCGUGAGAAUC | | |
| | | | CACGUGCAGAAGAA | | |
| | | | CAUCUGGACCUACC | | |
| | | | CUCUGUUCGUGAAC | | |
| | | | ACCUUCAGCGCGAA | | |
| | | | CGCCCUGGUGGGCC | | |
| | | | UGAGCAGCUGCAGC | | |
| | | | GCCACCCAGUGCUU | | |
| | | | CGGCCCCAAGCACC | | |
| | | | AUCACCACCACCAC | | |
| SEQ ID NO: | | 123 | 124 | 3 | 4 |

SEQ ID NO: 222 consists of from 5′ end to 3′ end, 5′ UTR SEQ
ID NO: 3, mRNA ORF SEQ ID NO: 124, and 3′ UTR SEQ ID NO: 4.

| | St_PltB | METPAQLLFLLLLWL | AUGGAGACACCUGC | GGGAAA | UGAUAA |
|---|---|---|---|---|---|
| | _NGM_nI | PDTTGEWTGDNTNA | CCAGCUGCUGUUCC | UAAGAG | UAGGCU |
| | gK | YYSDEVISELHVGQI | UGCUGCUGCUGUGG | AGAAAA | GGAGCC |
| | | DTSPYFCIKTVKANG | CUGCCUGACACCAC | GAAGAG | UCGGUG |
| | | AGTPVVACAVSKQSI | UGGCGAGUGGACCG | UAAGAA | GCCAUG |
| | | WAPSFKELLDQARYF | GCGACAACACCAAC | GAAAUA | CUUCUU |
| | | YSTGQSVRIHVQKNI | GCCUACUACAGCGA | UAAGAG | GCCCCU |
| | | WTYPLFVNTFSANAL | CGAGGUGAUCUCCG | CCACC | UGGGCC |
| | | VGLSSCSATQCFGPK | AGCUGCACGUGGGA | | UCCCCC |
| | | | CAGAUCGACACCAG | | CAGCCC |
| | | | CCCUUACUUCUGCA | | CUCCUC |
| | | | UCAAGACCGUGAAG | | CCCUUC |
| | | | GCCAACGGCGCAGG | | CUGCAC |
| | | | CACCCCUGUGGUGG | | CCGUAC |
| | | | CCUGCGCCGUGAGC | | CCCCGU |
| | | | AAGCAGAGCAUCUG | | GGUCUU |
| | | | GGCCCCUAGCUUCA | | UGAAUA |
| | | | AGGAGCUGCUGGAC | | AAGUCU |
| | | | CAGGCCAGAUACUU | | GAGUGG |
| | | | CUACAGCACCGGCC | | GCGGC |
| | | | AGAGCGUGAGAAUC | | |
| | | | CACGUGCAGAAGAA | | |
| | | | CAUCUGGACCUACC | | |
| | | | CUCUGUUCGUGAAC | | |
| | | | ACCUUCAGCGCGAA | | |
| | | | CGCCCUGGUGGGCC | | |
| | | | UGAGCAGCUGCAGC | | |
| | | | GCCACCCAGUGCUU | | |
| | | | CGGCCCCAAG | | |
| SEQ ID NO: | | 125 | 126 | 3 | 4 |

SEQ ID NO: 223 consists of from 5′ end to 3′ end, 5′ UTR SEQ
ID NO: 3, mRNA ORF SEQ ID NO: 126, and 3′ UTR SEQ ID NO: 4.

| | St_PltB | METPAQLLFLLLLWL | AUGGAGACACCUGC | GGGAAA | UGAUAA |
|---|---|---|---|---|---|
| | _NGM_nI | PDTTGEWTGDNTNA | CCAGCUGCUGUUCC | UAAGAG | UAGGCU |
| | gK_cHis | YYSDEVISELHVGQI | UGCUGCUGCUGUGG | AGAAAA | GGAGCC |
| | | DTSPYFCIKTVKANG | CUGCCUGACACCAC | GAAGAG | UCGGUG |
| | | AGTPVVACAVSKQSI | UGGCGAGUGGACCG | UAAGAA | GCCAUG |
| | | WAPSFKELLDQARYF | GCGACAACACCAAC | GAAAUA | CUUCUU |
| | | YSTGQSVRIHVQKNI | GCCUACUACAGCGA | UAAGAG | GCCCCU |
| | | WTYPLFVNTFSANAL | CGAGGUGAUCUCCG | CCACC | UGGGCC |
| | | VGLSSCSATQCFGPK | AGCUGCACGUGGGA | | UCCCCC |
| | | HHHHHH | CAGAUCGACACCAG | | CAGCCC |
| | | | CCCUUACUUCUGCA | | CUCCUC |
| | | | UCAAGACCGUGAAG | | CCCUUC |
| | | | GCCAACGGCGCAGG | | CUGCAC |
| | | | CACCCCUGUGGUGG | | CCGUAC |
| | | | CCUGCGCCGUGAGC | | CCCCGU |
| | | | AAGCAGAGCAUCUG | | GGUCUU |
| | | | GGCCCCUAGCUUCA | | UGAAUA |
| | | | AGGAGCUGCUGGAC | | AAGUCU |
| | | | CAGGCCAGAUACUU | | GAGUGG |
| | | | CUACAGCACCGGCC | | GCGGC |
| | | | AGAGCGUGAGAAUC | | |
| | | | CACGUGCAGAAGAA | | |
| | | | CAUCUGGACCUACC | | |
| | | | CUCUGUUCGUGAAC | | |
| | | | ACCUUCAGCGCGAA | | |
| | | | CGCCCUGGUGGGCC | | |
| | | | UGAGCAGCUGCAGC | | |
| | | | GCCACCCAGUGCUU | | |
| | | | CGGCCCCAAGCACC | | |
| | | | AUCACCACCACCAC | | |
| SEQ ID NO: | | 127 | 128 | 3 | 129 |

SEQ ID NO: 224 consists of from 5′ end to 3′ end, 5′ UTR SEQ
ID NO: 3, mRNA ORF SEQ ID NO: 128, and 3′ UTR SEQ ID NO: 129.

| | St_pilL_nI | METPAQLLFLLLLWL | AUGGAAACCCCUGC | GGGAAA | UGAUAA |
|---|---|---|---|---|---|
| | gk | PDTTGMNTQALFCLS | CCAGCUGCUGUUCC | UAAGAG | UAGGCU |
| | | LPLVIAGCAQQTSTQ | UGCUGCUGCUGUGG | AGAAAA | GGAGCC |
| | | TARDSAFAQSQQPSV | CUGCCUGACACCAC | GAAGAG | UCGGUG |
| | | FSDIYQRSPEVTRSGR | CGGCAUGAACACCC | UAAGAA | GCCUAG |
| | | YTLVSIKSADAQREP | AGGCCCUGUUCUGC | GAAAUA | CUUCUU |
| | | LNQLIDITMPGQLVN | CUGAGCCUGCCUCU | UAAGAG | GCCCCU |
| | | SVGDGFRYLLFQSGY | CGUCAUCGCCGGUU | CCACC | UGGGCC |
| | | SLCGHYGADFSELLN | GCGCCCAGCAGACC | | UCCCCC |
| | | RPLPAVQRKIGPMRL | AGCACCCAGACCGC | | CAGCCC |
| | | SEALQVVAGPAWRM | CAGAGACAGCGCCU | | CUCCUC |
| | | SVDEVNREVCFVLRD | UCGCCCAGAGCCAG | | CCCUUC |
| | | AYRVQAKTKALTSV | CAGCCUAGCGUGUU | | CUGCAC |
| | | TPAVTGAARTSSPET | CAGCGACAUCUACC | | CCGUAC |
| | | TRNPYTGVLPGNKAE | AGAGAAGCCCUGAG | | CCCCGU |
| | | PVIAGQYLAVKKGTE | GUGACCAGAAGCGG | | GGUCUU |
| | | LKPVSQSLPVPALRP | CAGAUACACCCUGG | | UGAAUA |
| | | GGTPVTSGVSSAPPV | UGAGCAUCAAGAGC | | AAGUCU |
| | | KQVSSPAPRNPFTGK | GCCGACGCCCAGAG | | GAGUGG |
| | | NLTGTTLSASHSPAP | AGAGCCUCUGAACC | | GCGGC |
| | | VGEKAQAKTPSPSTV | AGCUGAUCGACAUC | | |
| | | KAVNPATATVLATTT | ACCAUGCCUGGCCA | | |
| | | KPLTGTPVAAVASGP | GCUGGUGAACAGCG | | |
| | | EWKAVVGSTLKESLT | UGGGCGACGGCUUC | | |
| | | DWAGRADCPGGGH | AGAUACCUGCUGUU | | |
| | | WVVIWQTPTDYRIDA | CCAGAGCGGCUACA | | |
| | | PLVFKGNFETALVQV | GCCUGUGCGGCCAC | | |
| | | FDLYKKADKPLFAEA | UACGGAGCCGACUU | | |
| | | SRLQCLVSVADKPAD | CAGCGAGCUGCUGA | | |
| | | RS | ACAGACCUCUGCCU | | |
| | | | GCCGUGCAGAGAAA | | |
| | | | GAUCGGCCCUAUGA | | |
| | | | GACUGAGCGAAGCC | | |
| | | | CUGCAGGUGGUCGC | | |
| | | | GGGCCCUGCCUGGA | | |
| | | | GAAUGAGCGUGGAC | | |
| | | | GAGGUGAACAGAGA | | |
| | | | GGUGUGCUUCGUGC | | |
| | | | UGAGAGACGCCUAC | | |
| | | | AGAGUGCAGGCCAA | | |
| | | | GACCAAGGCCCUGA | | |
| | | | CCAGCGUGACUCCA | | |
| | | | GCCGUUACCGGCGC | | |
| | | | CGCCCGGACAUCCU | | |
| | | | CCCCUGAGACUACC | | |
| | | | AGAAACCCUUACAC | | |
| | | | CGGCGUGCUGCCUG | | |
| | | | GCAACAAGGCCGAG | | |
| | | | CCCGUGAUCGCCGG | | |
| | | | CCAGUACCUGGCCG | | |
| | | | UGAAGAAGGGCACC | | |
| | | | GAGCUGAAGCCUGU | | |
| | | | GAGCCAGAGCCUGC | | |
| | | | CUGUGCCUGCCCUG | | |
| | | | AGGCCUGGCGGCAC | | |
| | | | CCCUGUGACCUCGG | | |
| | | | GCGUGUCCUCUGCC | | |
| | | | CCUCCUGUGAAGCA | | |
| | | | GGUGAGCAGCCCCG | | |
| | | | CCCCUAGAAACCCU | | |
| | | | UUCACCGGCAAGAA | | |
| | | | CCUGACCGGCACCA | | |
| | | | CCCUGAGCGCCAGC | | |
| | | | CACAGUCCCGCUCC | | |
| | | | GGUGGGCGAGAAGG | | |
| | | | CCCAGGCAAAGACG | | |
| | | | CCUAGCCCUAGCAC | | |
| | | | CGUGAAGGCCGUGA | | |
| | | | ACCCUGCCACCGCC | | |
| | | | ACCGUGCUCGCCAC | | |
| | | | CACCACUAAGCCUC | | |
| | | | UGACCGGAACUCCC | | |
| | | | GUGGCCGCCGUAGC | | |
| | | | CAGCGGACCUGAGU | | |
| | | | GGAAAGCUGUCGUG | | |
| | | | GGCUCGACCCUGAA | | |
| | | | GGAGAGCCUGACCG | | |
| | | | ACUGGGCCGGCAGA | | |
| | | | GCCGAUUGCCCCGG | | |
| | | | CGGCGGGCACUGGG | | |
| | | | UGGUGAUCUGGCAG | | |
| | | | ACCCCUACCGACUA | | |
| | | | CAGAAUCGACGCCC | | |
| | | | CUCUGGUGUUCAAG | | |
| | | | GGCAACUUCGAGAC | | |
| | | | AGCCCUGGUGCAGG | | |
| | | | UGUUCGACCUGUAC | | |
| | | | AAGAAGGCCGACAA | | |
| | | | GCCCCUGUUCGCCG | | |
| | | | AGGCCAGCAGACUG | | |
| | | | CAGUGCCUGGUGUC | | |
| | | | CGUGGCCGACAAAC | | |
| | | | CCGCCGACAGAAGC | | |
| SEQ ID NO: | | 130 | 131 | 3 | 129 |

SEQ ID NO: 225 consists of from 5′ end to 3′ end, 5′ UTR SEQ
ID NO: 3, mRNA ORF SEQ ID NO: 131, and 3′ UTR SEQ ID NO: 129.

| | St_pilL_N | METPAQLLFLLLLWL | AUGGAAACCCCUGC | GGGAAA | UGAUAA |
|---|---|---|---|---|---|
| | GM_nIgk | PDTTGMNTQALFCLS | CCAGCUGCUGUUCC | UAAGAG | UAGGCU |
| | | LPLVIAGCAQQTSTQ | UGCUGCUGCUGUGG | AGAAAA | GGAGCC |
| | | TARDSAFAQSQQPSV | CUGCCUGACACCAC | GAAGAG | UCGGUG |
| | | FSDIYQRSPEVTRSGR | CGGCAUGAACACCC | UAAGAA | GCCUAG |
| | | YTLVSIKSADAQREP | AGGCCCUGUUCUGC | GAAAUA | CUUCUU |
| | | LNQLIDITMPGQLVN | CUGAGCCUGCCUCU | UAAGAG | GCCCCU |
| | | SVGDGFRYLLFQSGY | CGUCAUCGCCGGUU | CCACC | UGGGCC |
| | | SLCGHYGADFSELLN | GCGCCCAGCAGACC | | UCCCCC |
| | | RPLPAVQRKIGPMRL | AGCACCCAGACCGC | | CAGCCC |
| | | SEALQVVAGPAWRM | CAGAGACAGCGCCU | | CUCCUC |
| | | SVDEVNREVCFVLRD | UCGCCCAGAGCCAG | | CCCUUC |
| | | AYRVQAKTKALTSV | CAGCCUAGCGUGUU | | CUGCAC |
| | | TPAVTGAARTSSPET | CAGCGACAUCUACC | | CCGUAC |
| | | TRNPYTGVLPGNKAE | AGAGAAGCCCUGAG | | CCCCGU |
| | | PVIAGQYLAVKKGTE | GUGACCAGAAGCGG | | GGUCUU |
| | | LKPVSQSLPVPALRP | CAGAUACACCCUGG | | UGAAUA |
| | | GGTPVTSGVSSAPPV | UGAGCAUCAAGAGC | | AAGUCU |
| | | KQVSSPAPRNPFTGK | GCCGACGCCCAGAG | | GAGUGG |
| | | NLAGTTLSASHSPAP | AGAGCCUCUGAACC | | GCGGC |
| | | VGEKAQAKTPSPSTV | AGCUGAUCGACAUC | | |
| | | KAVNPATATVLATTT | ACCAUGCCUGGCCA | | |
| | | KPLTGTPVAAVASGP | GCUGGUGAACAGCG | | |
| | | EWKAVVGSTLKESLT | UGGGCGACGGCUUC | | |
| | | DWAGRADCPGGGH | AGAUACCUGCUGUU | | |
| | | WVVIWQTPTDYRIDA | CCAGAGCGGCUACA | | |
| | | PLVFKGNFETALVQV | GCCUGUGCGGCCAC | | |
| | | FDLYKKADKPLFAEA | UACGGAGCCGACUU | | |
| | | SRLQCLVSVADKPAD | CAGCGAGCUGCUGA | | |
| | | RS | ACAGACCUCUGCCU | | |
| | | | GCCGUGCAGAGAAA | | |
| | | | GAUCGGCCCUAUGA | | |
| | | | GACUGAGCGAAGCC | | |
| | | | CUGCAGGUGGUCGC | | |
| | | | GGGCCCUGCCUGGA | | |
| | | | GAAUGAGCGUGGAC | | |
| | | | GAGGUGAACAGAGA | | |
| | | | GGUGUGCUUCGUGC | | |
| | | | UGAGAGACGCCUAC | | |
| | | | AGAGUGCAGGCCAA | | |
| | | | GACCAAGGCCCUGA | | |
| | | | CCAGCGUGACUCCA | | |
| | | | GCCGUUACCGGCGC | | |
| | | | CGCCCGGACAUCCU | | |
| | | | CCCCUGAGACUACC | | |
| | | | AGAAACCCUUACAC | | |
| | | | CGGCGUGCUGCCUG | | |
| | | | GCAACAAGGCCGAG | | |
| | | | CCCGUGAUCGCCGG | | |
| | | | CCAGUACCUGGCCG | | |
| | | | UGAAGAAGGGCACC | | |
| | | | GAGCUGAAGCCUGU | | |
| | | | GAGCCAGAGCCUGC | | |
| | | | CUGUGCCUGCCCUG | | |
| | | | AGGCCUGGCGGCAC | | |
| | | | CCCUGUGACCUCGG | | |
| | | | GCGUGUCCUCUGCC | | |
| | | | CCUCCUGUGAAGCA | | |
| | | | GGUGAGCAGCCCCG | | |
| | | | CCCCUAGAAACCCU | | |
| | | | UUCACCGGCAAGAA | | |
| | | | CCUGGCCGGCACCA | | |
| | | | CCCUGAGCGCCAGC | | |
| | | | CACAGUCCCGCUCC | | |
| | | | GGUGGGCGAGAAGG | | |
| | | | CCCAGGCAAAGACG | | |
| | | | CCUAGCCCUAGCAC | | |
| | | | CGUGAAGGCCGUGA | | |
| | | | ACCCUGCCACCGCC | | |
| | | | ACCGUGCUCGCCAC | | |
| | | | CACCACUAAGCCUC | | |
| | | | UGACCGGAACUCCC | | |
| | | | GUGGCCGCCGUAGC | | |
| | | | CAGCGGACCUGAGU | | |
| | | | GGAAAGCUGUCGUG | | |
| | | | GGCUCGACCCUGAA | | |
| | | | GGAGAGCCUGACCG | | |
| | | | ACUGGGCCGGCAGA | | |
| | | | GCCGAUUGCCCCGG | | |
| | | | CGGCGGGCACUGGG | | |
| | | | UGGUGAUCUGGCAG | | |
| | | | ACCCCUACCGACUA | | |
| | | | CAGAAUCGACGCCC | | |
| | | | CUCUGGUGUUCAAG | | |
| | | | GGCAACUUCGAGAC | | |
| | | | AGCCCUGGUGCAGG | | |
| | | | UGUUCGACCUGUAC | | |
| | | | AAGAAGGCCGACAA | | |
| | | | GCCCCUGUUCGCCG | | |
| | | | AGGCCAGCAGACUG | | |
| | | | CAGUGCCUGGUGUC | | |
| | | | CGUGGCCGACAAAC | | |
| | | | CCGCCGACAGAAGC | | |
| SEQ ID NO: | | 132 | 133 | 3 | 129 |

SEQ ID NO: 226 consists of from 5′ end to 3′ end, 5′ UTR SEQ
ID NO: 3, mRNA ORF SEQ ID NO: 133, and 3′ UTR SEQ ID NO: 129.

| | St_T0937_ | METPAQLLFLLLLWL | AUGGAGACUCCUGC | GGGAAA | UGAUAA |
|---|---|---|---|---|---|
| | nIgK_NG | PDTTGQTTNRSTAKD | CCAGCUGCUGUUCC | UAAGAG | UAGGCU |
| | M_cHis | EILTGSYIDPTKTRFP | UGCUGCUGCUGUGG | AGAAAA | GGAGCC |
| | | LADYSQSVDKWIPPD | CUGCCUGACACCAC | GAAGAG | UCGGUG |
| | | SADYTIPVIDSATQQR | CGGCCAGACCACCA | UAAGAA | GCCUAG |
| | | YFSALKSHYFGMDSE | ACAGAAGCACCGCC | GAAAUA | CUUCUU |
| | | AHSPWNDFYITALLK | AAGGACGAGAUCCU | UAAGAG | GCCCCU |
| | | KNAAQARDASIKQFL | GACCGGCAGCUACA | CCACC | UGGGCC |
| | | SDGSAYWGENFRLY | UCGACCCUACCAAG | | UCCCCC |
| | | TSRWKEEVRGNTDT | ACCAGAUUUCCCCU | | CAGCCC |
| | | QIDNIYHASRRGIMV | GGCUGAUUAUAGCC | | CUCCUC |
| | | RESLVRALPTDDPLF | AGAGCGUGGACAAG | | CCCUUC |
| | | NDPRQAGEGYPFDNL | UGGAUCCCUCCUGA | | CUGCAC |
| | | QMSSLRPGTPVYTLT | CUCAGCCGACUACA | | CCGUAC |
| | | KSKDQRWQYVVSPA | CCAUCCCUGUGAUC | | CCCCGU |
| | | VTGWVHSEDIASTDQ | GACAGCGCCACCCA | | GGUCUU |
| | | KFITQWVLLAHKQLG | GCAGAGAUACUUCA | | UGAAUA |
| | | AFINAPVSVHAAGVY | GCGCCCUGAAGUCC | | AAGUCU |
| | | YFTGRPGTILPFRHQR | CACUACUUCGGCAU | | GAGUGG |
| | | AGQFLIAAPVRGSNG | GGACAGCGAGGCCC | | GCGGC |
| | | RAFIHWVWLSGNEFT | ACAGCCCUUGGAAC | | |
| | | AMPWKMTPENIAVL | GACUUCUACAUCAC | | |
| | | MKAMHGAPYGWGN | CGCCCUGCUGAAGA | | |
| | | FNFYNDCSAEVRSLL | AGAACGCCGCCCAG | | |
| | | MPFGIFLPRHSSAQVE | GCCAGAGACGCCAG | | |
| | | SAGRVVDLSHKNPQ | CAUCAAGCAGUUUC | | |
| | | MRIDYLTRYGKAFTT | UGUCCGACGGCAGC | | |
| | | LVYIPGHIMLYIGNTA | GCCUACUGGGGCGA | | |
| | | MNGQVVPMTYQNIW | GAACUUCAGACUGU | | |
| | | GLRPNHANSRSIIGEA | ACACCAGCCGGUGG | | |
| | | VFLPLLRFYPENPELI | AAGGAGGAGGUGA | | |
| | | SLAGKVLFKLGYIEH | GAGGCAACACCGAC | | |
| | | HHHHH | ACCCAGAUCGACAA | | |
| | | | CAUCUACCACGCCA | | |
| | | | GCCGAAGGGGCAUC | | |
| | | | AUGGUGAGAGAGA | | |
| | | | GCCUGGUGAGAGCC | | |
| | | | CUGCCUACCGACGA | | |
| | | | CCCUCUGUUCAACG | | |
| | | | ACCCUAGACAGGCC | | |
| | | | GGCGAGGGCUACCC | | |
| | | | UUUCGACAACCUGC | | |
| | | | AGAUGAGCAGCCUG | | |
| | | | CGCCCUGGCACCCC | | |
| | | | UGUGUACACCCUGA | | |
| | | | CCAAGAGCAAGGAC | | |
| | | | CAGCGGUGGCAGUA | | |
| | | | CGUGGUGUCCCCUG | | |
| | | | CCGUGACAGGGUGG | | |
| | | | GUGCACAGCGAGGA | | |
| | | | CAUCGCCAGCACCG | | |
| | | | ACCAGAAGUUCAUC | | |
| | | | ACCCAGUGGGUGCU | | |
| | | | GCUGGCCCAUAAGC | | |
| | | | AGCUCGGCGCCUUC | | |
| | | | AUCAACGCCCCUGU | | |
| | | | UUCCGUGCACGCCG | | |
| | | | CCGGCGUGUACUAC | | |
| | | | UUCACCGGCAGACC | | |
| | | | CGGAACCAUCCUGC | | |
| | | | CUUUCAGACACCAG | | |
| | | | AGAGCCGGGCAGUU | | |
| | | | CCUGAUAGCCGCCC | | |
| | | | CGGUACGCGGGAGC | | |
| | | | AACGGCCGGGCCUU | | |
| | | | UAUCCACUGGGUGU | | |
| | | | GGCUGAGCGGCAAC | | |
| | | | GAGUUCACCGCCAU | | |
| | | | GCCUUGGAAGAUGA | | |
| | | | CCCCUGAGAACAUC | | |
| | | | GCCGUCCUGAUGAA | | |
| | | | GGCCAUGCACGGCG | | |
| | | | CCCCUUACGGCUGG | | |
| | | | GGCAACUUCAACUU | | |
| | | | CUACAACGACUGCA | | |
| | | | GCGCCGAGGUGAGA | | |
| | | | AGCCUGCUGAUGCC | | |
| | | | UUUCGGCAUCUUCC | | |
| | | | UGCCCCGUCACUCC | | |
| | | | AGCGCCCAGGUCGA | | |
| | | | GAGCGCCGGCAGAG | | |
| | | | UGGUGGACCUGAGC | | |
| | | | CACAAGAACCCUCA | | |
| | | | GAUGAGAAUCGACU | | |
| | | | ACCUGACCAGAUAC | | |
| | | | GGCAAGGCCUUCAC | | |
| | | | CACACUCGUGUACA | | |
| | | | UCCCCGGCCACAUC | | |
| | | | AUGCUGUACAUCGG | | |
| | | | CAACACAGCCAUGA | | |
| | | | ACGGCCAGGUGGUG | | |
| | | | CCUAUGACCUACCA | | |
| | | | GAACAUCUGGGGCC | | |
| | | | UCAGACCUAACCAC | | |
| | | | GCCAACAGCAGAAG | | |
| | | | CAUCAUCGGCGAGG | | |
| | | | CCGUGUUCCUGCCU | | |
| | | | CUGCUGAGAUUCUA | | |
| | | | CCCUGAGAACCCUG | | |
| | | | AGCUGAUCAGCCUG | | |
| | | | GCCGGCAAGGUGCU | | |
| | | | GUUCAAGCUGGGCU | | |
| | | | ACAUCGAGCACCAU | | |
| | | | CACCACCACCAC | | |
| SEQ ID NO: | | 134 | 135 | 3 | 129 |

SEQ ID NO: 227 consists of from 5′ end to 3′ end, 5′ UTR SEQ
ID NO: 3, mRNA ORF SEQ ID NO: 135, and 3′ UTR SEQ ID NO: 129.

| | St_T0937_ | METPAQLLFLLLLWL | AUGGAGACUCCUGC | GGGAAA | UGAUAA |
|---|---|---|---|---|---|
| | nIgK_nTru | PDTTGVDKWIPPDSA | CCAGCUGCUGUUCC | UAAGAG | UAGGCU |
| | nc_NGM_ | DYTIPVIDSATQQRYF | UGCUGCUGCUGUGG | AGAAAA | GGAGCC |
| | cHis | SALKSHYFGMDSEA | CUGCCUGACACCAC | GAAGAG | UCGGUG |
| | | HSPWNDFYITALLKK | CGGCGUGGACAAGU | UAAGAA | GCCUAG |
| | | NAAQARDASIKQFLS | GGAUCCCUCCUGAC | GAAAUA | CUUCUU |
| | | DGSAYWGENFRLYT | UCAGCCGACUACAC | UAAGAG | GCCCCU |
| | | SRWKEEVRGNTDTQI | CAUCCCUGUGAUCG | CCACC | UGGGCC |
| | | DNIYHASRRGIMVRE | ACAGCGCCACCCAG | | UCCCCC |
| | | SLVRALPTDDPLFND | CAGAGAUACUUCAG | | CAGCCC |
| | | PRQAGEGYPFDNLQ | CGCCCUGAAGUCCC | | CUCCUC |
| | | MSSLRPGTPVYTLTK | ACUACUUCGGCAUG | | CCCUUC |
| | | SKDQRWQYVVSPAV | GACAGCGAGGCCCA | | CUGCAC |
| | | TGWVHSEDIASTDQK | CAGCCCUUGGAACG | | CCGUAC |
| | | FITQWVLLAHKQLGA | ACUUCUACAUCACC | | CCCCGU |
| | | FINAPVSVHAAGVYY | GCCCUGCUGAAGAA | | GGUCUU |
| | | FTGRPGTILPFRHQRA | GAACGCCGCCCAGG | | UGAAUA |
| | | GQFLIAAPVRGSNGR | CCAGAGACGCCAGC | | AAGUCU |
| | | AFIHWVWLSGNEFTA | AUCAAGCAGUUUCU | | GAGUGG |
| | | MPWKMTPENIAVLM | GUCCGACGGCAGCG | | GCGGC |
| | | KAMHGAPYGWGNF | CCUACUGGGGCGAG | | |
| | | NFYNDCSAEVRSLLM | AACUUCAGACUGUA | | |
| | | PFGIFLPRHSSAQVES | CACCAGCCGGUGGA | | |
| | | AGRVVDLSHKNPQM | AGGAGGAGGUGAG | | |
| | | RIDYLTRYGKAFTTL | AGGCAACACCGACA | | |
| | | VYIPGHIMLYIGNTA | CCCAGAUCGACAAC | | |
| | | MNGQVVPMTYQNIW | AUCUACCACGCCAG | | |
| | | GLRPNHANSRSIIGEA | CCGAAGGGGCAUCA | | |
| | | VFLPLLRFYPENPELI | UGGUGAGAGAGAGC | | |
| | | SLAGKVLFKLGYIEH | CUGGUGAGAGCCCU | | |
| | | HHHHH | GCCUACCGACGACC | | |
| | | | CUCUGUUCAACGAC | | |
| | | | CCUAGACAGGCCGG | | |
| | | | CGAGGGCUACCCUU | | |
| | | | UCGACAACCUGCAG | | |
| | | | AUGAGCAGCCUGCG | | |
| | | | CCCUGGCACCCCUG | | |
| | | | UGUACACCCUGACC | | |
| | | | AAGAGCAAGGACCA | | |
| | | | GCGGUGGCAGUACG | | |
| | | | UGGUGUCCCCUGCC | | |
| | | | GUGACAGGGUGGGU | | |
| | | | GCACAGCGAGGACA | | |
| | | | UCGCCAGCACCGAC | | |
| | | | CAGAAGUUCAUCAC | | |
| | | | CCAGUGGGUGCUGC | | |
| | | | UGGCCCAUAAGCAG | | |
| | | | CUCGGCGCCUUCAU | | |
| | | | CAACGCCCCUGUUU | | |
| | | | CCGUGCACGCCGCC | | |
| | | | GGCGUGUACUACUU | | |
| | | | CACCGGCAGACCCG | | |
| | | | GAACCAUCCUGCCU | | |
| | | | UUCAGACACCAGAG | | |
| | | | AGCCGGGCAGUUCC | | |
| | | | UGAUAGCCGCCCCG | | |
| | | | GUACGCGGGAGCAA | | |
| | | | CGGCCGGGCCUUUA | | |
| | | | UCCACUGGGUGUGG | | |
| | | | CUGAGCGGCAACGA | | |
| | | | GUUCACCGCCAUGC | | |
| | | | CUUGGAAGAUGACC | | |
| | | | CCUGAGAACAUCGC | | |
| | | | CGUCCUGAUGAAGG | | |
| | | | CCAUGCACGGCGCC | | |
| | | | CCUUACGGCUGGGG | | |
| | | | CAACUUCAACUUCU | | |
| | | | ACAACGACUGCAGC | | |
| | | | GCCGAGGUGAGAAG | | |
| | | | CCUGCUGAUGCCUU | | |
| | | | UCGGCAUCUUCCUG | | |
| | | | CCCCGUCACUCCAG | | |
| | | | CGCCCAGGUCGAGA | | |
| | | | GCGCCGGCAGAGUG | | |
| | | | GUGGACCUGAGCCA | | |
| | | | CAAGAACCCUCAGA | | |
| | | | UGAGAAUCGACUAC | | |
| | | | CUGACCAGAUACGG | | |
| | | | CAAGGCCUUCACCA | | |
| | | | CACUCGUGUACAUC | | |
| | | | CCCGGCCACAUCAU | | |
| | | | GCUGUACAUCGGCA | | |
| | | | ACACAGCCAUGAAC | | |
| | | | GGCCAGGUGGUGCC | | |
| | | | UAUGACCUACCAGA | | |
| | | | ACAUCUGGGGCCUC | | |
| | | | AGACCUAACCACGC | | |
| | | | CAACAGCAGAAGCA | | |
| | | | UCAUCGGCGAGGCC | | |
| | | | GUGUUCCUGCCUCU | | |
| | | | GCUGAGAUUCUACC | | |
| | | | CUGAGAACCCUGAG | | |
| | | | CUGAUCAGCCUGGC | | |
| | | | CGGCAAGGUGCUGU | | |
| | | | UCAAGCUGGGCUAC | | |
| | | | AUCGAGCACCAUCA | | |
| | | | CCACCACCAC | | |
| SEQ ID NO: | | 136 | 137 | 3 | 129 |

SEQ ID NO: 228 consists of from 5′ end to 3′ end, 5′ UTR SEQ
ID NO: 3, mRNA ORF SEQ ID NO: 137, and 3′ UTR SEQ ID NO: 129.

| | St_slyB_nI | METPAQLLFLLLLWL | AUGGAAACGCCUGC | GGGAAA | UGAUAA |
|---|---|---|---|---|---|
| | gK_NGM_ | PDTTGDSLSGDVYTA | CCAGCUGCUGUUCC | UAAGAG | UAGGCU |
| | cHis | SEAKQVQNVAYGTIV | UGCUGCUGCUGUGG | AGAAAA | GGAGCC |
| | | NVRPVQIQGGDDSNV | CUGCCUGACACCAC | GAAGAG | UCGGUG |
| | | IGAIGGAVLGGFLGN | CGGCGACAGCCUGA | UAAGAA | GCCUAG |
| | | TIGGGTGRSLATAAG | GCGGCGACGUGUAC | GAAAUA | CUUCUU |
| | | AVAGGVAGQGVQSA | ACCGCCAGCGAGGC | UAAGAG | GCCCCU |
| | | MNKAQGVELEIRKD | CAAGCAGGUGCAGA | CCACC | UGGGCC |
| | | DGNTIMVVQKQGNT | ACGUGGCCUACGGC | | UCCCCC |
| | | RFSAGQRVVLASNG | ACCAUCGUGAACGU | | CAGCCC |
| | | AQVTVSPRHHHHHH | CAGACCUGUGCAGA | | CUCCUC |
| | | | UCCAGGGCGGGGAC | | CCCUUC |
| | | | GACAGCAACGUGAU | | CUGCAC |
| | | | CGGCGCCAUUGGAG | | CCGUAC |
| | | | GUGCCGUGCUGGGC | | CCCCGU |
| | | | GGCUUCCUGGGCAA | | GGUCUU |
| | | | CACUAUCGGCGGCG | | UGAAUA |
| | | | GCACCGGCAGAAGC | | AAGUCU |
| | | | CUGGCCACAGCCGC | | GAGUGG |
| | | | CGGAGCCGUGGCUG | | GCGGC |
| | | | GCGGCGUGGCCGGU | | |
| | | | CAGGGAGUGCAGAG | | |
| | | | CGCCAUGAACAAGG | | |
| | | | CCCAGGGCGUGGAG | | |
| | | | CUGGAGAUCAGAAA | | |
| | | | GGACGACGGCAACA | | |
| | | | CCAUCAUGGUGGUG | | |
| | | | CAGAAGCAAGGGAA | | |
| | | | CACCAGAUUCAGCG | | |
| | | | CCGGCCAGAGAGUG | | |
| | | | GUGCUGGCCAGCAA | | |
| | | | CGGCGCCCAGGUGA | | |
| | | | CCGUGAGCCCUCGC | | |
| | | | CAUCACCACCACCA | | |
| | | | CCAC | | |
| SEQ ID NO: | | 138 | 139 | 140 | 129 |

SEQ ID NO: 229 consists of from 5′ end to 3′ end, 5′ UTR SEQ
ID NO: 140, mRNA ORF SEQ ID NO: 139, and 3′ UTR SEQ ID NO: 129.

| | St_CdtB_T | METPAQLLFLLLLWL | AUGGAGACUCCUGC | GGGAAA | UGAUAA |
|---|---|---|---|---|---|
| | runc_IgK_ | PDTTGYKVMTWNLQ | CCAGCUGCUGUUCC | UAAGAG | UAGGCU |
| | cHis | GSSASTESKWNVNV | UGCUGCUGCUGUGG | AGAAAA | GGAGCC |
| | | RQLLSGTAGVDILMV | CUGCCUGACACCAC | GAAGAG | UCGGUG |
| | | QEAGAVPTSAVPTGR | CGGCUACAAGGUGA | UAAGAA | GCCUAG |
| | | HIQPFGVGIPIDEYTW | UGACCUGGAACCUG | GAAAUA | CUUCUU |
| | | NLGTTSRQDIRYIYHS | CAGGGAAGCUCCGC | UAAGAC | GCCCCU |
| | | AIDVGARRVNLAIVS | CAGCACCGAGAGCA | CCCGGC | UGGGCC |
| | | RQRADNVYVLRPTT | AGUGGAACGUGAAC | GCCGCC | UCCCCC |
| | | VASRPVIGIGLGNDV | GUGAGACAGCUCCU | ACC | CAGCCC |
| | | FLTAHALASGGPDAA | GAGCGGCACCGCCG | | CUCCUC |
| | | AIVRVTINFFRQPQM | GCGUCGACAUCCUG | | CCCUUC |
| | | RHLSWFLAGDFNRSP | AUGGUGCAGGAGGC | | CUGCAC |
| | | DRLENDLMTEHLER | CGGAGCCGUCCCUA | | CCGUAC |
| | | VVAVLAPTEPTQIGG | CCAGCGCCGUGCCU | | CCCCGU |
| | | GILDYGVIVDRAPYS | ACCGGCAGACACAU | | GGUCUU |
| | | QRVEALRNPQLASDH | CCAGCCUUUCGGCG | | UGAAUA |
| | | YPVAFLHHHHHH | UGGGCAUCCCUAUC | | AAGUCU |
| | | | GACGAGUACACCUG | | GAGUGG |
| | | | GAAUCUCGGCACCA | | GCGGC |
| | | | CCAGCAGACAGGAC | | |
| | | | AUCAGAUACAUCUA | | |
| | | | CCACAGCGCCAUCG | | |
| | | | ACGUGGGCGCCAGA | | |
| | | | AGAGUGAACCUGGC | | |
| | | | CAUCGUGAGCAGAC | | |
| | | | AGAGAGCCGACAAC | | |
| | | | GUGUACGUGCUGAG | | |
| | | | GCCUACCACCGUGG | | |
| | | | CCAGCAGACCUGUG | | |
| | | | AUCGGCAUCGGCCU | | |
| | | | GGGCAACGACGUGU | | |
| | | | UCCUGACCGCCCAC | | |
| | | | GCUCUGGCCUCCGG | | |
| | | | UGGCCCCGACGCUG | | |
| | | | CCGCCAUCGUGAGA | | |
| | | | GUGACCAUCAACUU | | |
| | | | CUUCAGACAGCCUC | | |
| | | | AGAUGAGACACCUG | | |
| | | | AGCUGGUUCCUGGC | | |
| | | | CGGCGACUUCAACA | | |
| | | | GAAGCCCUGACAGA | | |
| | | | CUGGAGAACGACCU | | |
| | | | GAUGACCGAGCACC | | |
| | | | UGGAGAGAGUGGU | | |
| | | | GGCCGUGCUGGCCC | | |
| | | | CUACCGAGCCUACC | | |
| | | | CAGAUCGGCGGCGG | | |
| | | | CAUCCUGGACUACG | | |
| | | | GCGUGAUCGUGGAC | | |
| | | | AGAGCCCCUUACAG | | |
| | | | CCAGAGAGUGGAGG | | |
| | | | CCCUGAGAAACCCU | | |
| | | | CAGCUGGCCAGCGA | | |
| | | | CCACUACCCUGUGG | | |
| | | | CCUUCCUGCAUCAC | | |
| | | | CACCACCACCAC | | |
| SEQ ID NO: | | 141 | 142 | 140 | 129 |

SEQ ID NO: 230 consists of from 5′ end to 3′ end, 5′ UTR SEQ
ID NO: 140, mRNA ORF SEQ ID NO: 142, and 3′ UTR SEQ ID NO: 129.

| | St_CdtB_T | METPAQLLFLLLLWL | AUGGAGACUCCUGC | GGGAAA | UGAUAA |
|---|---|---|---|---|---|
| | runc_H160 | PDTTGYKVMTWNLQ | CCAGCUGCUGUUCC | UAAGAG | UAGGCU |
| | Q_IgK_cH | GSSASTESKWNVNV | UGCUGCUGCUGUGG | AGAAAA | GGAGCC |
| | is | RQLLSGTAGVDILMV | CUGCCUGACACCAC | GAAGAG | UCGGUG |
| | | QEAGAVPTSAVPTGR | CGGCUACAAGGUGA | UAAGAA | GCCUAG |
| | | HIQPFGVGIPIDEYTW | UGACCUGGAACCUG | GAAAUA | CUUCUU |
| | | NLGTTSRQDIRYIYHS | CAGGGAAGCUCCGC | UAAGAC | GCCCCU |
| | | AIDVGARRVNLAIVS | CAGCACCGAGAGCA | CCCGGC | UGGGCC |
| | | RQRADNVYVLRPTT | AGUGGAACGUGAAC | GCCGCC | UCCCCC |
| | | VASRPVIGIGLGNDV | GUGAGACAGCUCCU | ACC | CAGCCC |
| | | FLTAQALASGGPDAA | GAGCGGCACCGCCG | | CUCCUC |
| | | AIVRVTINFFRQPQM | GCGUCGACAUCCUG | | CCCUUC |
| | | RHLSWFLAGDFNRSP | AUGGUGCAGGAGGC | | CUGCAC |
| | | DRLENDLMTEHLER | CGGAGCCGUCCCUA | | CCGUAC |
| | | VVAVLAPTEPTQIGG | CCAGCGCCGUGCCU | | CCCCGU |
| | | GILDYGVIVDRAPYS | ACCGGCAGACACAU | | GGUCUU |
| | | QRVEALRNPQLASDH | CCAGCCUUUCGGCG | | UGAAUA |
| | | YPVAFLHHHHHH | UGGGCAUCCCUAUC | | AAGUCU |
| | | | GACGAGUACACCUG | | GAGUGG |
| | | | GAAUCUCGGCACCA | | GCGGC |
| | | | CCAGCAGACAGGAC | | |
| | | | AUCAGAUACAUCUA | | |
| | | | CCACAGCGCCAUCG | | |
| | | | ACGUGGGCGCCAGA | | |
| | | | AGAGUGAACCUGGC | | |
| | | | CAUCGUGAGCAGAC | | |
| | | | AGAGAGCCGACAAC | | |
| | | | GUGUACGUGCUGAG | | |
| | | | GCCUACCACCGUGG | | |
| | | | CCAGCAGACCUGUG | | |
| | | | AUCGGCAUCGGCCU | | |
| | | | GGGCAACGACGUGU | | |
| | | | UCCUGACCGCCCAG | | |
| | | | GCUCUGGCCUCCGG | | |
| | | | UGGCCCCGACGCUG | | |
| | | | CCGCCAUCGUGAGA | | |
| | | | GUGACCAUCAACUU | | |
| | | | CUUCAGACAGCCUC | | |
| | | | AGAUGAGACACCUG | | |
| | | | AGCUGGUUCCUGGC | | |
| | | | CGGCGACUUCAACA | | |
| | | | GAAGCCCUGACAGA | | |
| | | | CUGGAGAACGACCU | | |
| | | | GAUGACCGAGCACC | | |
| | | | UGGAGAGAGUGGU | | |
| | | | GGCCGUGCUGGCCC | | |
| | | | CUACCGAGCCUACC | | |
| | | | CAGAUCGGCGGCGG | | |
| | | | CAUCCUGGACUACG | | |
| | | | GCGUGAUCGUGGAC | | |
| | | | AGAGCCCCUUACAG | | |
| | | | CCAGAGAGUGGAGG | | |
| | | | CCCUGAGAAACCCU | | |
| | | | CAGCUGGCCAGCGA | | |
| | | | CCACUACCCUGUGG | | |
| | | | CCUUCCUGCAUCAC | | |
| | | | CACCACCACCAC | | |
| SEQ ID NO: | | 143 | 144 | 140 | 129 |

SEQ ID NO: 231 consists of from 5′ end to 3′ end, 5′ UTR SEQ
ID NO: 140, mRNA ORF SEQ ID NO: 144, and 3′ UTR SEQ ID NO: 129.

| | St_STY07 | METPAQLLFLLLLWL | AUGGAGACACCUGC | GGGAAA | UGAUAA |
|---|---|---|---|---|---|
| | 96_nIgK_c | PDTTGQAPISSVGSGS | CCAGCUGCUGUUCC | UAAGAG | UAGGCU |
| | His | VEDRVTQLERISNAH | UGCUGCUGCUGUGG | AGAAAA | GGAGCC |
| | | SQLLTQLQQQLSDNQ | CUGCCCGACACCAC | GAAGAG | UCGGUG |
| | | SDIDSLRGQIQENQY | CGGCCAGGCCCCUA | UAAGAA | GCCUAG |
| | | QLNQVMERQKQIML | UCAGCAGCGUGGGC | GAAAUA | CUUCUU |
| | | QLGSLNNGGAAQPA | AGCGGAAGCGUGGA | UAAGAC | GCCCCU |
| | | AGDQSGAATAATPA | GGACAGAGUGACCC | CCCGGC | UGGGCC |
| | | PDAGTATSGAPVQSG | AGCUGGAGAGAAUC | GCCGCC | UCCCCC |
| | | DANTDYNAAIALVQ | AGCAACGCCCACAG | ACC | CAGCCC |
| | | DKSRQDDAIVAFQNF | CCAGCUGCUCACCC | | CUCCUC |
| | | IKKYPDSTYQPNANY | AGCUCCAGCAGCAG | | CCCUUC |
| | | WLGQLNYNKGKKD | CUGAGCGACAACCA | | CUGCAC |
| | | DAAYYFASVVKNYP | GAGCGACAUCGACA | | CCGUAC |
| | | KSPKAADAMYKVGV | GCCUGAGAGGCCAG | | CCCCGU |
| | | IMQDKGDTAKAKAV | AUCCAGGAGAACCA | | GGUCUU |
| | | YQQVINKYPGTDGA | GUACCAGCUGAACC | | UGAAUA |
| | | KQAQKRLNAMHHH | AGGUGAUGGAGAG | | AAGUCU |
| | | HHH | ACAGAAGCAGAUCA | | GAGUGG |
| | | | UGCUGCAGCUGGGA | | GCGGC |
| | | | AGCCUGAACAACGG | | |
| | | | CGGAGCCGCUCAGC | | |
| | | | CUGCAGCCGGCGAC | | |
| | | | CAGAGUGGCGCCGC | | |
| | | | AACAGCCGCCACAC | | |
| | | | CAGCCCCUGACGCG | | |
| | | | GGUACAGCCACCAG | | |
| | | | CGGUGCUCCCGUGC | | |
| | | | AGAGCGGCGACGCC | | |
| | | | AACACCGACUACAA | | |
| | | | CGCCGCCAUCGCCC | | |
| | | | UGGUGCAGGACAAG | | |
| | | | AGCAGACAGGACGA | | |
| | | | CGCUAUCGUGGCCU | | |
| | | | UCCAGAACUUCAUC | | |
| | | | AAGAAGUACCCCGA | | |
| | | | CAGCACCUACCAGC | | |
| | | | CCAACGCCAACUAC | | |
| | | | UGGCUGGGCCAGCU | | |
| | | | GAACUACAACAAGG | | |
| | | | GCAAGAAGGACGAC | | |
| | | | GCCGCCUACUACUU | | |
| | | | CGCCAGCGUGGUGA | | |
| | | | AGAACUACCCCAAG | | |
| | | | AGCCCCAAAGCGGC | | |
| | | | CGACGCCAUGUACA | | |
| | | | AGGUGGGCGUGAUC | | |
| | | | AUGCAGGACAAGGG | | |
| | | | CGACACCGCCAAGG | | |
| | | | CCAAGGCCGUGUAC | | |
| | | | CAGCAGGUGAUCAA | | |
| | | | CAAGUACCCCGGUA | | |
| | | | CUGACGGCGCCAAG | | |
| | | | CAGGCCCAGAAGAG | | |
| | | | ACUGAACGCCAUGC | | |
| | | | ACCAUCACCAUCAC | | |
| | | | CAC | | |
| SEQ ID NO: | | 145 | 146 | 140 | 129 |

SEQ ID NO: 232 consists of from 5′ end to 3′ end, 5′ UTR SEQ
ID NO: 140, mRNA ORF SEQ ID NO: 146, and 3′ UTR SEQ ID NO: 129.

| | St_STY07 | METPAQLLFLLLLWL | AUGGAGACACCUGC | GGGAAA | UGAUAA |
|---|---|---|---|---|---|
| | 96_NGM_ | PDTTGQAPISSVGSGS | CCAGCUGCUGUUCC | UAAGAG | UAGGCU |
| | nIgK_cHis | VEDRVTQLERISNAH | UGCUGCUGCUGUGG | AGAAAA | GGAGCC |
| | | SQLLTQLQQQLSDNQ | CUGCCCGACACCAC | GAAGAG | UCGGUG |
| | | ADIDSLRGQIQENQY | CGGCCAGGCCCCUA | UAAGAA | GCCUAG |
| | | QLNQVMERQKQIML | UCAGCAGCGUGGGC | GAAAUA | CUUCUU |
| | | QLGSLNNGGAAQPA | AGCGGAAGCGUGGA | UAAGAC | GCCCCU |
| | | AGDQSGAATAATPA | GGACAGAGUGACCC | CCCGGC | UGGGCC |
| | | PDAGTATSGAPVQSG | AGCUGGAGAGAAUC | GCCGCC | UCCCCC |
| | | DANTDYNAAIALVQ | AGCAACGCCCACAG | ACC | CAGCCC |
| | | DKSRQDDAIVAFQNF | CCAGCUGCUCACCC | | CUCCUC |
| | | IKKYPDSTYQPNANY | AGCUCCAGCAGCAG | | CCCUUC |
| | | WLGQLNYNKGKKD | CUGAGCGACAACCA | | CUGCAC |
| | | DAAYYFASVVKNYP | GGCCGACAUCGACA | | CCGUAC |
| | | KSPKAADAMYKVGV | GCCUGAGAGGCCAG | | CCCCGU |
| | | IMQDKGDTAKAKAV | AUCCAGGAGAACCA | | GGUCUU |
| | | YQQVINKYPGTDGA | GUACCAGCUGAACC | | UGAAUA |
| | | KQAQKRLNAMHHH | AGGUGAUGGAGAG | | AAGUCU |
| | | HHH | ACAGAAGCAGAUCA | | GAGUGG |
| | | | UGCUGCAGCUGGGA | | GCGGC |
| | | | AGCCUGAACAACGG | | |
| | | | CGGAGCCGCUCAGC | | |
| | | | CUGCAGCCGGCGAC | | |
| | | | CAGAGUGGCGCCGC | | |
| | | | AACAGCCGCCACAC | | |
| | | | CAGCCCCUGACGCG | | |
| | | | GGUACAGCCACCAG | | |
| | | | CGGUGCUCCCGUGC | | |
| | | | AGAGCGGCGACGCC | | |
| | | | AACACCGACUACAA | | |
| | | | CGCCGCCAUCGCCC | | |
| | | | UGGUGCAGGACAAG | | |
| | | | AGCAGACAGGACGA | | |
| | | | CGCUAUCGUGGCCU | | |
| | | | UCCAGAACUUCAUC | | |
| | | | AAGAAGUACCCCGA | | |
| | | | CAGCACCUACCAGC | | |
| | | | CCAACGCCAACUAC | | |
| | | | UGGCUGGGCCAGCU | | |
| | | | GAACUACAACAAGG | | |
| | | | GCAAGAAGGACGAC | | |
| | | | GCCGCCUACUACUU | | |
| | | | CGCCAGCGUGGUGA | | |
| | | | AGAACUACCCCAAG | | |
| | | | AGCCCCAAAGCGGC | | |
| | | | CGACGCCAUGUACA | | |
| | | | AGGUGGGCGUGAUC | | |
| | | | AUGCAGGACAAGGG | | |
| | | | CGACACCGCCAAGG | | |
| | | | CCAAGGCCGUGUAC | | |
| | | | CAGCAGGUGAUCAA | | |
| | | | CAAGUACCCCGGUA | | |
| | | | CUGACGGCGCCAAG | | |
| | | | CAGGCCCAGAAGAG | | |
| | | | ACUGAACGCCAUGC | | |
| | | | ACCAUCACCAUCAC | | |
| | | | CAC | | |
| SEQ ID NO: | | 147 | 148 | 140 | 129 |

SEQ ID NO: 233 consists of from 5′ end to 3′ end, 5′ UTR SEQ
ID NO: 140, mRNA ORF SEQ ID NO: 148, and 3′ UTR SEQ ID NO: 129.

| | St_STY10 | METPAQLLFLLLLWL | AUGGAAACCCCAGC | GGGAAA | UGAUAA |
|---|---|---|---|---|---|
| | 86_nIgK_c | PDTTGENKSYYQLPI | CCAGCUCCUGUUCC | UAAGAG | UAGGCU |
| | His | AQSGVQSTASQGNRL | UGCUGCUGCUGUGG | AGAAAA | GGAGCC |
| | | LWVEQVSVPDYLAG | CUUCCCGACACCAC | GAAGAG | UCGGUG |
| | | NGVVYQTSDVQYVI | CGGCGAGAACAAGA | UAAGAA | GCCUAG |
| | | ANNNLWASPLDQQL | GCUACUACCAGCUG | GAAAUA | CUUCUU |
| | | RNTLVANLSARLPG | CCCAUCGCCCAGAG | UAAGAC | GCCCCU |
| | | WVVASQPLGTTQDT | CGGCGUGCAGAGCA | CCCGGC | UGGGCC |
| | | LNVTVTGFHGRYDG | CAGCCAGCCAGGGC | GCCGCC | UCCCCC |
| | | KVIVSGEWLLNHNG | AACAGACUGCUGUG | ACC | CAGCCC |
| | | QLIKRPFHIEASQQKD | GGUGGAGCAGGUGA | | CUCCUC |
| | | GYDEMVKVLASAWS | GCGUGCCCGACUAC | | CCCUUC |
| | | QEAAAIADEIKRLPH | CUGGCCGGCAACGG | | CUGCAC |
| | | HHHHH | CGUGGUGUACCAGA | | CCGUAC |
| | | | CCAGCGACGUGCAG | | CCCCGU |
| | | | UACGUGAUCGCCAA | | GGUCUU |
| | | | CAACAACCUGUGGG | | UGAAUA |
| | | | CCAGCCCGCUGGAC | | AAGUCU |
| | | | CAGCAGCUGAGAAA | | GAGUGG |
| | | | CACCCUGGUGGCCA | | GCGGC |
| | | | ACCUGAGCGCCAGA | | |
| | | | CUCCCCGGCUGGGU | | |
| | | | CGUUGCCUCCCAGC | | |
| | | | CCCUGGGCACCACC | | |
| | | | CAGGACACCCUGAA | | |
| | | | CGUGACCGUGACCG | | |
| | | | GCUUCCACGGCAGA | | |
| | | | UACGACGGCAAGGU | | |
| | | | GAUCGUGAGCGGCG | | |
| | | | AGUGGCUGCUGAAC | | |
| | | | CACAACGGCCAGCU | | |
| | | | GAUCAAGAGGCCCU | | |
| | | | UCCACAUCGAGGCC | | |
| | | | AGCCAGCAGAAGGA | | |
| | | | CGGCUACGACGAGA | | |
| | | | UGGUGAAGGUGCUG | | |
| | | | GCCAGCGCCUGGAG | | |
| | | | CCAGGAGGCCGCUG | | |
| | | | CCAUAGCCGACGAG | | |
| | | | AUCAAGAGACUGCC | | |
| | | | CCACCAUCACCACC | | |
| | | | ACCAC | | |
| SEQ ID NO: | | 149 | 150 | 140 | 129 |

SEQ ID NO: 234 consists of from 5′ end to 3′ end, 5′ UTR SEQ
ID NO: 140, mRNA ORF SEQ ID NO: 150, and 3′ UTR SEQ ID NO: 129.

| | | | | |
|---|---|---|---|---|
| St_STY10 | METPAQLLFLLLLWL | AUGGAAACCCCAGC | GGGAAA | UGAUAA |
| 86_NGM_ | PDTTGENKAYYQLPI | CCAGCUCCUGUUCC | UAAGAG | UAGGCU |
| nIgK_cHis | AQSGVQSTASQGNRL | UGCUGCUGCUGUGG | AGAAAA | GGAGCC |
| | LWVEQVSVPDYLAG | CUUCCCGACACCAC | GAAGAG | UCGGUG |
| | NGVVYQTSDVQYVI | CGGCGAGAACAAGG | UAAGAA | GCCUAG |
| | ANNNLWASPLDQQL | CCUACUACCAGCUG | GAAAUA | CUUCUU |
| | RNTLVANLAARLPG | CCCAUCGCCCAGAG | UAAGAC | GCCCCU |
| | WVVASQPLGTTQDT | CGGCGUGCAGAGCA | CCCGGC | UGGGCC |
| | LNVAVTGFHGRYDG | CAGCCAGCCAGGGC | GCCGCC | UCCCCC |
| | KVIVSGEWLLNHNG | AACAGACUGCUGUG | ACC | CAGCCC |
| | QLIKRPFHIEASQQKD | GGUGGAGCAGGUGA | | CUCCUC |
| | GYDEMVKVLASAWS | GCGUGCCCGACUAC | | CCCUUC |
| | QEAAAIADEIKRLPH | CUGGCCGGCAACGG | | CUGCAC |
| | HHHHH | CGUGGUGUACCAGA | | CCGUAC |
| | | CCAGCGACGUGCAG | | CCCCGU |
| | | UACGUGAUCGCCAA | | GGUCUU |
| | | CAACAACCUGUGGG | | UGAAUA |
| | | CCAGCCCGCUGGAC | | AAGUCU |
| | | CAGCAGCUGAGAAA | | GAGUGG |
| | | CACCCUGGUGGCCA | | GCGGC |
| | | ACCUGGCCGCCAGA | | |
| | | CUCCCCGGCUGGGU | | |
| | | CGUUGCCUCCCAGC | | |
| | | CCCUGGGCACCACC | | |
| | | CAGGACACCCUGAA | | |
| | | CGUGGCCGUGACCG | | |
| | | GCUUCCACGGCAGA | | |
| | | UACGACGGCAAGGU | | |
| | | GAUCGUGAGCGGCG | | |
| | | AGUGGCUGCUGAAC | | |
| | | CACAACGGCCAGCU | | |
| | | GAUCAAGAGGCCCU | | |
| | | UCCACAUCGAGGCC | | |
| | | AGCCAGCAGAAGGA | | |
| | | CGGCUACGACGAGA | | |
| | | UGGUGAAGGUGCUG | | |
| | | GCCAGCGCCUGGAG | | |
| | | CCAGGAGGCCGCUG | | |
| | | CCAUAGCCGACGAG | | |
| | | AUCAAGAGACUGCC | | |
| | | CCACCAUCACCACC | | |
| | | ACCAC | | |

1. A multivalent Salmonella vaccine, comprising: (a) a messenger RNA (mRNA) comprising an open reading frame (ORF) encoding a first Salmonella antigen, and (b) a mRNA comprising an ORF encoding a second Salmonella antigen, formulated in an ionizable cationic lipid nanoparticle that comprises a molar ratio of 20-60% ionizable cationic lipid, 5-25% non-cationic lipid, 25-55% sterol, and 0.5-15% PEG-modified lipid, wherein the first and second Salmonella antigens are selected from the group consisting of: SseB, Mig14, OmpL, OmpC, OmpD, OmpF, iroN, cirA, FepA, T0937, FliC, PilL, PltB, PltA, CdtB, SlyB, STY1086 and STY0796, and,
wherein intramuscular (IM) administration of a therapeutically effective amount of the vaccine to a subject induces in the subject a neutralizing antibody titer and/or a T cell immune response.

2.-7. (canceled)

8. The vaccine of claim 1, wherein the Salmonella antigens comprise PltB, PltA, CdtB.

9. The vaccine of claim 1, wherein each Salmonella antigen is of a different serotype.

10. The vaccine of claim 9, wherein the serotypes are selected from the group consisting of: enterica (serotype I), salamae (serotype II), arizonae (Ma), diarizonae (Mb), houtenae (IV), and indica (VI).

11. The vaccine of claim 1, wherein the Salmonella antigens are fused to a scaffold moiety.

12. (canceled)

13. The vaccine of claim 1, wherein the neutralizing antibody titer is at least 100 neutralizing units per milliliter (U/ml).

14. The vaccine of claim 13, wherein the neutralizing antibody titer is at least 500 U/ml.

15. The vaccine of claim 14, wherein the neutralizing antibody titer is at least 1000 U/ml.

16. The vaccine of claim 1, wherein the Salmonella antigen is expressed on the surface of cells of the subject.

17. The vaccine of claim 1, wherein the neutralizing antibody titer is induced within 20 days following a single 10-100 μg of the vaccine.

18. The vaccine of claim 1, wherein the neutralizing antibody titer is induced within 40 days following a second 10-100 μg dose of the vaccine.

19. The vaccine of claim 1, wherein the T cell immune response comprises a CD4+ T cell immune response and/or a CD8+ T cell immune response.

20. (canceled)

21. (canceled)

22. The vaccine of any one of claims 1-21, wherein the RNA further comprises a 5′ UTR and/or a 3′ UTR.

23. The vaccine of claim 22, wherein the 5′ UTR comprises a sequence identified by SEQ ID NO:3 or SEQ ID NO:140.

24. (canceled)

25. The vaccine of claim 22, wherein the 3′ UTR comprises a sequence identified by SEQ ID NO:4 or SEQ ID NO:129.

26. The vaccine of claim 1, wherein the Salmonella antigen is fused to a signal peptide.

27. The vaccine of claim 26, wherein the signal peptide is selected from the group consisting of SEQ ID NO: 151-156.

28. (canceled)

29. The vaccine of claim 1, wherein the RNA comprise at least one modified nucleotide.

30. The vaccine of claim 29, wherein at least 80% of the uracil in the ORF comprise 1-methyl-pseudouridine modification.

31. A method comprising administering to a subject the Salmonella vaccine of claim 1 in a therapeutically effective amount to induce in the subject a neutralizing antibody titer and/or a T cell immune response.

32.-38. (canceled)
